SEQUENCE LISTING (172 sequences)

SEQ ID NO: 1 - DNA, 290 bp, Escherichia coli
  misc_feature 1..290: note = FNR Responsive Promoter
  source 1..290: mol_type = genomic DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 2 - DNA, 173 bp, Escherichia coli
  misc_feature 1..173: note = FNR Responsive Promoter
  source 1..173: mol_type = genomic DNA
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 3 - DNA, 305 bp, Escherichia coli
  misc_feature 1..305: note = FNR Responsive Promoter
  source 1..305: mol_type = genomic DNA
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 4 - DNA, 180 bp, Escherichia coli
  misc_feature 1..180: note = FNR Responsive Promoter
  source 1..180: mol_type = genomic DNA
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 5 - DNA, 199 bp, Escherichia coli
  misc_feature 1..199: note = FNR Responsive Promoter
  source 1..199: mol_type = genomic DNA
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 6 - DNA, 1647 bp, Lactococcus lactis
  misc_feature 1..1647: note = kivD (Lactococcus lactis IFPL730)
  source 1..1647: mol_type = genomic DNA
atgtatacagtaggagattacctattagaccgattacacgagttaggaattgaagaaatttttggagtccctggagactataacttacaatttttagatcaaattatttcccacaaggatatgaaatgggtcggaaatgctaatgaattaaatgcttcatatatggctgatggctatgctcgtactaaaaaagctgccgcatttcttacaacctttggagtaggtgaattgagtgcagttaatggattagcaggaagttacgccgaaaatttaccagtagtagaaatagtgggatcacctacatcaaaagttcaaaatgaaggaaaatttgttcatcatacgctggctgacggtgattttaaacactttatgaaaatgcacgaacctgttacagcagctcgaactttactgacagcagaaaatgcaaccgttgaaattgaccgagtactttctgcactattaaaagaaagaaaacctgtctatatcaacttaccagttgatgttgctgctgcaaaagcagagaaaccctcactccctttgaaaaaggaaaactcaacttcaaatacaagtgaccaagaaattttgaacaaaattcaagaaagcttgaaaaatgccaaaaaaccaatcgtgattacaggacatgaaataattagttttggcttagaaaaaacagtcactcaatttatttcaaagacaaaactacctattacgacattaaactttggtaaaagttcagttgatgaagccctcccttcatttttaggaatctataatggtacactctcagagcctaatcttaaagaattcgtggaatcagccgacttcatcttgatgcttggagttaaactcacagactcttcaacaggagccttcactcatcatttaaatgaaaataaaatgatttcactgaatatagatgaaggaaaaatatttaacgaaagaatccaaaattttgattttgaatccctcatctcctctctcttagacctaagcgaaatagaatacaaaggaaaatatatcgataaaaagcaagaagactttgttccatcaaatgcgcttttatcacaagaccgcctatggcaagcagttgaaaacctaactcaaagcaatgaaacaatcgttgctgaacaagggacatcattctttggcgcttcatcaattttcttaaaatcaaagagtcattttattggtcaacccttatggggatcaattggatatacattcccagcagcattaggaagccaaattgcagataaagaaagcagacaccttttatttattggtgatggttcacttcaacttacagtgcaagaattaggattagcaatcagagaaaaaattaatccaatttgctttattatcaataatgatggttatacagtcgaaagagaaattcatggaccaaatcaaagctacaatgatattccaatgtggaattactcaaaattaccagaatcgtttggagcaacagaagatcgagtagtctcaaaaatcgttagaactgaaaatgaatttgtgtctgtcatgaaagaagctcaagcagatccaaatagaatgtactggattgagttaattttggcaaaagaaggtgcaccaaaagtactgaaaaaaatgggcaaactatttgctgaacaaaataaatcataa

SEQ ID NO: 7 - DNA, 5739 bp, synthetic construct
  misc_feature 1..5739: note = Synthetic: Tet-bkd construct sequence
  source 1..5739: mol_type = other DNA
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgagtgattacgagccgttgcgtctgcatgtcccggagcccaccgggcgtcctggctgcaagaccgacttttcctatctgcacctgtcccccgccggcgaggtacgcaagccgccggtggatgtcgagcccgccgaaaccagcgacctggcctacagcctggtacgtgtgctcgacgacgacggccacgccgtcggtccctggaatccgcagctcagcaacgaacaactgctgcgcggcatgcgggcgatgctcaagacccgcctgttcgacgcgcgcatgctcaccgcgcaacggcagaaaaagctttccttctatatgcaatgcctcggcgaggaagccatcgccaccgcccacaccctggccctgcgcgacggcgacatgtgctttccgacctatcgccagcaaggcatcctgatcacccgcgaatacccgctggtggacatgatctgccagcttctctccaacgaggccgacccgctcaagggccgccagctgccgatcatgtactcgagcaaggaggcaggtttcttctccatctccggcaacctcgccacccagttcatccaggcggtcggctggggcatggcctcggcgatcaagggcgacacgcgcatcgcctcggcctggatcggcgacggcgccaccgccgagtcggacttccacaccgccctcaccttcgcccatgtctaccgcgcgccggtaatcctcaacgtggtcaacaaccagtgggcgatctccaccttccaggccatcgccggcggcgaaggcaccaccttcgccaaccgtggcgtgggctgcgggatcgcctcgctgcgggtcgacggcaatgacttcctggcggtctacgccgcctccgagtgggccgccgagcgcgcccggcgcaacctcgggccgagcctgatcgaatgggtcacctaccgcgccggcccgcactcgacttcggacgacccgtccaagtaccgccccgccgacgactggaccaacttcccgctgggcgacccgatcgcccgcctgaagcggcacatgatcggcctcggcatctggtcggaggaacagcacgaagccacccacaaggccctcgaagccgaagtactggccgcgcagaaacaggcggagagccatggcaccctgatcgacggccgggtgccgagcgccgccagcatgttcgaggacgtctatgcagaactgccggagcatctgcgccggcaacgccaggagctcggggtatgaatgccatgaacccgcaacacgagaacgcccagacggtcaccagcatgaccatgatccaggcgctgcgctcggcgatggacatcatgctcgagcgcgacgacgacgtggtggtattcggccaggacgtcggctacttcggcggcgtgttccgctgcaccgaaggcctgcagaagaaatacggcacctcgcgggtgttcgatgcgccgatctccgagagcggcatcatcggcgccgcggtcggcatgggtgcctacggcctgcgcccggtggtggagatccagttcgccgactacgtctacccggcctccgaccagttgatctccgaggcggcgcgcctgcgctatcgctcggccggcgacttcatcgtgccgatgaccgtacgcatgccctgtggcggcggcatctacggcgggcaaacgcacagccagagcccggaggcgatgttcacccaggtctgcggcctgcgcacggtgatgccgtccaacccctacgacgccaagggcctgctgatcgcctgcatcgagaacgacgacccggtgatcttcctcgagcccaagcgcctctacaacggcccgttcgatggccaccacgaccgcccggtgacgccctggtccaagcatccggccagccaggtgccggacggctactacaaggtgccgctggacaaggcggcgatcgtccgccccggcgcggcgctgaccgtgctgacctacggcaccatggtctacgtggcccaggccgcggccgacgaaaccggcctggacgccgagatcatcgacctgcgcagcctctggccgctggacctggaaaccatcgtcgcctcggtgaagaagaccggccgctgcgtcatcgcccacgaggcgacccgcacctgtgggttcggcgccgagctgatgtcgctggtgcaggagcactgcttccaccacctggaggcgccgatcgagcgcgtcaccggttgggacaccccctacccgcatgcccaggagtgggcgtatttccccggccccgcgcgcgtcggcgcggcattcaagcgtgtgatggaggtctgaatgggtacccatgtgatcaagatgccggacatcggggaaggcatcgccgaggtcgaactggtggagtggcatgtccaggtcggcgactcggtcaatgaagaccaggtcctcgccgaggtgatgaccgacaaggccacggtggagattccctcgccggtggccggacgcatcctcgccctcggcggccagccgggccaggtgatggcggtgggcggcgaactgatccgcctggaggtggaaggcgccggcaacctcgccgagagtccggccgcggcgacgccggccgcgcccgtcgccgccaccccggagaaaccgaaggaagccccggtcgcggcgccgaaagccgccgccgaagcgccgcgcgccttgcgcgacagcgaggcgccacggcagcggcgccagcccggcgaacgcccgctggcctcccccgcggtgcgccagcgcgcccgcgacctgggcatcgagttgcagttcgtgcagggcagcggtcccgccggacgcgtcctccacgaggacctcgatgcctacctgacccaggatggcagcgtcgcgcgcagcggcggcgccgcgcaggggtatgccgagcgacacgacgaacaggcggtgccggtgatcggcctgcgtcgcaagatcgcccagaagatgcaggacgccaagcgacgcatcccgcatttcagctatgtcgaggaaatcgacgtcaccgatctggaagccctgcgcgcccatctcaaccagaaatggggtggccagcgcggcaagctgaccctgctgccgttcctggtccgcgccatggtcgtggcgctgcgcgacttcccgcagttgaacgcgcgctacgacgacgaggccgaggtggtcacccgctacggcgcggtgcacgtcggcatcgccacccagagcgacaacggcctgatggtgccggtgctgcgccacgccgaatcgcgcgacctctggggcaacgccagcgaagtggcgcgcctggccgaagccgcacgcagcggcaaggcgcaacgccaggagctgtccggctcgaccatcaccctgagcagcctcggcgtgctcggcgggatcgtcagcacaccggtgatcaaccatccggaggtggccatcgtcggcgtcaaccgcatcgtcgagcgaccgatggtggtcggcggcaacatcgtcgtgcgcaagatgatgaacctctcctcctccttcgaccaccgggtggtcgacgggatggacgcggcggccttcatccaggccgtgcgcggcctgctcgaacatcccgccaccctgttcctggagtaagcgatgagccagatcctgaagacttccctgctgatcgtcggcggcggtcccggcggctacgtcgcggcgatccgtgccgggcaactgggcattcccaccgtactggtggagggcgccgccctcggcggcacctgtctgaacgtcggctgcatcccgtcgaaggcgctgatccacgccgccgaggaatacctcaaggcccgccactatgccagccggtcggcgctgggcatccaggtacaggcgccgagcatcgacatcgcccgcaccgtggaatggaaggacgccatcgtcgaccgcctcaccagcggcgtcgccgcgctgctgaagaaacacggggtcgatgtcgtccagggctgggcgaggatcctcgacggcaaaagcgtggcggtcgaactcgccggcggcggcagccagcgcatcgagtgcgagcatctgctgctggccgccggctcgcagagcgtcgagctaccgatcctgccgctgggcggcaaggtgatctcctccaccgaggcgctggcgcccggcagcctgcccaagcgcctggtggtggtcggcggcggctacatcggcctggagctgggtaccgcctaccgcaagctcggcgtcgaggtggcggtggtggaagcgcaaccacgcatcctgccgggctacgacgaagaactgaccaagccggtggcccaggccttgcgcaggctgggcgtcgagctgtacctcgggcacagcctgctgggcccgagcgagaacggcgtgcgggtccgcgacggcgccggcgaggagcgcgagatcgccgccgaccaggtactggtggcggtcggccgcaagccgcgcagcgaaggctggaacctggaaagcctgggcctggacatgaacggccgggcggtgaaggtcgacgaccagtgccgcacctcgatgcgcaatgtctgggccataggcgatctcgccggcgagccgatgctcgcgcaccgggccatggcccagggcgagatggtcgccgagctgatcgccggcaagcgtcgccagttcgccccggtggcgatccccgcggtgtgcttcaccgatccggaagtggtggtcgccgggttgtccccggagcaggcgaaggatgccggcctggactgcctggtggcgagcttcccgttcgccgccaacggtcgcgccatgaccctggaggccaacgaaggcttcgtccgcgtggtggcgcgtcgcgacaaccacctggtcgtcggctggcaggcggtgggcaaggcggtttcggaactgtccacggccttcgcccagtcgctggagatgggcgcccgcctggaagacatcgccggcaccatccacgcccatccgaccctcggcgaagcggtccaggaagccgccctgcgcgcgctgggacacgccctgcacatctga

SEQ ID NO: 8 - DNA, 6780 bp, synthetic construct
  misc_feature 1..6780: note = Synthetic: Tet-ldh-bkd construct
  source 1..6780: mol_type = other DNA
gtaaaacgacggccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgttcgacatgatggacgcggcccggctcgagggtctgcacctcgcccaagacccggccacgggactcaaggccattatcgccatccacagcacgcgactcggcccggcgctgggtggttgtcgctacctgccttaccccaacgacgaagccgccatcggcgacgccatccgcctggcccagggcatgagctacaaggcggccctggccgggctggagcagggcggcggcaaggcggtgatcatccgcccgccgcacctggacaatcgcggcgcgctgttcgaggccttcgggcgcttcatcgaaagcctcggcggacgctacatcactgcggtggacagcggtacctccagcgccgacatggactgcatcgcccagcagacccgccacgtcaccagcaccacccaggccggcgacccctcgccgcataccgccctcggcgtgttcgccgggattcgcgccagcgcccaggcgcgcctcggcagcgacgacctggaaggcctgcgggtcgcggtgcaggggctcggccacgtcggctacgcattggccgagcaactggcggcggtcggcgccgagctgctggtctgcgacctcgatcccggccgggtgcaactggccgtcgagcagctcggtgcccatccgctggcgccggaggcattgctctccaccccttgcgacatcctcgcgccctgcggcctgggcggcgtgctcaccagccagagcgtcagccagttgcgctgcgcggcggtggccggggcggcgaacaaccagttggagcggccggaggtcgccgacgagctggaggcgcgcggcatcctctatgcgccggactacgtgatcaactccggcggcctgatctacgtcgccctcaagcaccgcggcgccgatccgcacagcatcaccgcgcacctggcgcggattcccgcgcggctcaccgagatctatgcccatgcccaggccgaccaccagtcgccggcgcggatcgccgaccgtctggcggaacggattctctacggcccgcagtgagaaggagatatacatatgagtgattacgagccgttgcgtctgcatgtcccggagcccaccgggcgtcctggctgcaagaccgacttttcctatctgcacctgtcccccgccggcgaggtacgcaagccgccggtggatgtcgagcccgccgaaaccagcgacctggcctacagcctggtacgtgtgctcgacgacgacggccacgccgtcggtccctggaatccgcagctcagcaacgaacaactgctgcgcggcatgcgggcgatgctcaagacccgcctgttcgacgcgcgcatgctcaccgcgcaacggcagaaaaagctttccttctatatgcaatgcctcggcgaggaagccatcgccaccgcccacaccctggccctgcgcgacggcgacatgtgctttccgacctatcgccagcaaggcatcctgatcacccgcgaatacccgctggtggacatgatctgccagcttctctccaacgaggccgacccgctcaagggccgccagctgccgatcatgtactcgagcaaggaggcaggtttcttctccatctccggcaacctcgccacccagttcatccaggcggtcggctggggcatggcctcggcgatcaagggcgacacgcgcatcgcctcggcctggatcggcgacggcgccaccgccgagtcggacttccacaccgccctcaccttcgcccatgtctaccgcgcgccggtaatcctcaacgtggtcaacaaccagtgggcgatctccaccttccaggccatcgccggcggcgaaggcaccaccttcgccaaccgtggcgtgggctgcgggatcgcctcgctgcgggtcgacggcaatgacttcctggcggtctacgccgcctccgagtgggccgccgagcgcgcccggcgcaacctcgggccgagcctgatcgaatgggtcacctaccgcgccggcccgcactcgacttcggacgacccgtccaagtaccgccccgccgacgactggaccaacttcccgctgggcgacccgatcgcccgcctgaagcggcacatgatcggcctcggcatctggtcggaggaacagcacgaagccacccacaaggccctcgaagccgaagtactggccgcgcagaaacaggcggagagccatggcaccctgatcgacggccgggtgccgagcgccgccagcatgttcgaggacgtctatgcagaactgccggagcatctgcgccggcaacgccaggagctcggggtatgaatgccatgaacccgcaacacgagaacgcccagacggtcaccagcatgaccatgatccaggcgctgcgctcggcgatggacatcatgctcgagcgcgacgacgacgtggtggtattcggccaggacgtcggctacttcggcggcgtgttccgctgcaccgaaggcctgcagaagaaatacggcacctcgcgggtgttcgatgcgccgatctccgagagcggcatcatcggcgccgcggtcggcatgggtgcctacggcctgcgcccggtggtggagatccagttcgccgactacgtctacccggcctccgaccagttgatctccgaggcggcgcgcctgcgctatcgctcggccggcgacttcatcgtgccgatgaccgtacgcatgccctgtggcggcggcatctacggcgggcaaacgcacagccagagcccggaggcgatgttcacccaggtctgcggcctgcgcacggtgatgccgtccaacccctacgacgccaagggcctgctgatcgcctgcatcgagaacgacgacccggtgatcttcctcgagcccaagcgcctctacaacggcccgttcgatggccaccacgaccgcccggtgacgccctggtccaagcatccggccagccaggtgccggacggctactacaaggtgccgctggacaaggcggcgatcgtccgccccggcgcggcgctgaccgtgctgacctacggcaccatggtctacgtggcccaggccgcggccgacgaaaccggcctggacgccgagatcatcgacctgcgcagcctctggccgctggacctggaaaccatcgtcgcctcggtgaagaagaccggccgctgcgtcatcgcccacgaggcgacccgcacctgtgggttcggcgccgagctgatgtcgctggtgcaggagcactgcttccaccacctggaggcgccgatcgagcgcgtcaccggttgggacaccccctacccgcatgcccaggagtgggcgtatttccccggccccgcgcgcgtcggcgcggcattcaagcgtgtgatggaggtctgaatgggtacccatgtgatcaagatgccggacatcggggaaggcatcgccgaggtcgaactggtggagtggcatgtccaggtcggcgactcggtcaatgaagaccaggtcctcgccgaggtgatgaccgacaaggccacggtggagattccctcgccggtggccggacgcatcctcgccctcggcggccagccgggccaggtgatggcggtgggcggcgaactgatccgcctggaggtggaaggcgccggcaacctcgccgagagtccggccgcggcgacgccggccgcgcccgtcgccgccaccccggagaaaccgaaggaagccccggtcgcggcgccgaaagccgccgccgaagcgccgcgcgccttgcgcgacagcgaggcgccacggcagcggcgccagcccggcgaacgcccgctggcctcccccgcggtgcgccagcgcgcccgcgacctgggcatcgagttgcagttcgtgcagggcagcggtcccgccggacgcgtcctccacgaggacctcgatgcctacctgacccaggatggcagcgtcgcgcgcagcggcggcgccgcgcaggggtatgccgagcgacacgacgaacaggcggtgccggtgatcggcctgcgtcgcaagatcgcccagaagatgcaggacgccaagcgacgcatcccgcatttcagctatgtcgaggaaatcgacgtcaccgatctggaagccctgcgcgcccatctcaaccagaaatggggtggccagcgcggcaagctgaccctgctgccgttcctggtccgcgccatggtcgtggcgctgcgcgacttcccgcagttgaacgcgcgctacgacgacgaggccgaggtggtcacccgctacggcgcggtgcacgtcggcatcgccacccagagcgacaacggcctgatggtgccggtgctgcgccacgccgaatcgcgcgacctctggggcaacgccagcgaagtggcgcgcctggccgaagccgcacgcagcggcaaggcgcaacgccaggagctgtccggctcgaccatcaccctgagcagcctcggcgtgctcggcgggatcgtcagcacaccggtgatcaaccatccggaggtggccatcgtcggcgtcaaccgcatcgtcgagcgaccgatggtggtcggcggcaacatcgtcgtgcgcaagatgatgaacctctcctcctccttcgaccaccgggtggtcgacgggatggacgcggcggccttcatccaggccgtgcgcggcctgctcgaacatcccgccaccctgttcctggagtaagcgatgagccagatcctgaagacttccctgctgatcgtcggcggcggtcccggcggctacgtcgcggcgatccgtgccgggcaactgggcattcccaccgtactggtggagggcgccgccctcggcggcacctgtctgaacgtcggctgcatcccgtcgaaggcgctgatccacgccgccgaggaatacctcaaggcccgccactatgccagccggtcggcgctgggcatccaggtacaggcgccgagcatcgacatcgcccgcaccgtggaatggaaggacgccatcgtcgaccgcctcaccagcggcgtcgccgcgctgctgaagaaacacggggtcgatgtcgtccagggctgggcgaggatcctcgacggcaaaagcgtggcggtcgaactcgccggcggcggcagccagcgcatcgagtgcgagcatctgctgctggccgccggctcgcagagcgtcgagctaccgatcctgccgctgggcggcaaggtgatctcctccaccgaggcgctggcgcccggcagcctgcccaagcgcctggtggtggtcggcggcggctacatcggcctggagctgggtaccgcctaccgcaagctcggcgtcgaggtggcggtggtggaagcgcaaccacgcatcctgccgggctacgacgaagaactgaccaagccggtggcccaggccttgcgcaggctgggcgtcgagctgtacctcgggcacagcctgctgggcccgagcgagaacggcgtgcgggtccgcgacggcgccggcgaggagcgcgagatcgccgccgaccaggtactggtggcggtcggccgcaagccgcgcagcgaaggctggaacctggaaagcctgggcctggacatgaacggccgggcggtgaaggtcgacgaccagtgccgcacctcgatgcgcaatgtctgggccataggcgatctcgccggcgagccgatgctcgcgcaccgggccatggcccagggcgagatggtcgccgagctgatcgccggcaagcgtcgccagttcgccccggtggcgatccccgcggtgtgcttcaccgatccggaagtggtggtcgccgggttgtccccggagcaggcgaaggatgccggcctggactgcctggtggcgagcttcccgttcgccgccaacggtcgcgccatgaccctggaggccaacgaaggcttcgtccgcgtggtggcgcgtcgcgacaaccacctggtcgtcggctggcaggcggtgggcaaggcggtttcggaactgtccacggccttcgcccagtcgctggagatgggcgcccgcctggaagacatcgccggcaccatccacgcccatccgaccctcggcgaagcggtccaggaagccgccctgcgcgcgctgggacacgccctgcacatctga

SEQ ID NO: 9 - DNA, 5597 bp, synthetic construct
  misc_feature 1..5597: note = Synthetic: Tet-livKHMGF construct
  source 1..5597: mol_type = other DNA
ccagtgaattcgttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtgatcatcccaccgcccgtaaaatgcgggcgggtttagaaaggttaccttatgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtgtcattcgccctgctgattctggtgctgctggtgatgccgaccggtattctgggtcgcccggaggtagagaaagtatgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctggggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataagatggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 10 - DNA, 1104 bp, Escherichia coli
  misc_feature 1..1104: note = livJ (Escherichia coli)
  source 1..1104: mol_type = genomic DNA
atgaacataaagggtaaagcgttactggcaggatgtatcgcgctggcattcagcaatatggctctggcagaagatattaaagtcgcggtcgtgggcgcaatgtccggtccggttgcgcagtacggtgaccaggagtttaccggcgcagagcaggcggttgcggatatcaacgctaaaggcggcattaaaggcaacaaactgcaaatcgtaaaatatgacgatgcctgtgacccgaaacaggcggttgcggtggcgaacaaagtcgttaacgacggcattaaatatgtgattggtcacctctgttcttcatcaacgcagcctgcgtctgacatctacgaagacgaaggcattttaatgatcaccccagcggcaaccgcgccggagctgaccgcccgtggctatcagctgatcctgcgcaccaccggcctggactccgaccaggggccgacggcggcgaaatatattcttgagaaagtgaaaccgcagcgtattgctatcgttcacgacaaacagcaatacggcgaaggtctggcgcgagcggtgcaggacggcctgaagaaaggcaatgcaaacgtggtgttctttgatggcatcaccgccggggaaaaagatttctcaacgctggtggcgcgtctgaaaaaagagaatatcgacttcgtttactacggcggttatcacccggaaatggggcaaatcctgcgtcaggcacgcgcggcagggctgaaaactcagtttatggggccggaaggtgtggctaacgtttcgctgtctaacattgcgggcgaatcagcggaagggctgctggtgaccaagccgaagaactacgatcaggttccggcgaacaaacccattgttgacgcgatcaaagcgaaaaaacaggacccaagtggcgcattcgtttggaccacctacgccgcgctgcaatctttgcaggcgggcctgaatcagtctgacgatccggctgaaatcgccaaatacctgaaagcgaactccgtggataccgtaatgggaccgctgacctgggatgagaaaggcgatctgaaaggctttgagttcggcgtatttgactggcacgccaacggcacggcgaccgatgcgaagtaa

SEQ ID NO: 11 - DNA, 639 bp, Escherichia coli
  misc_feature 1..639: note = leucine exporter gene leuE (Escherichia coli Nissle 1917)
  source 1..639: mol_type = genomic DNA
gtgttcgctgaatacggggttctgaattactggacctatctggttggggccatttttattgtgttggtgccagggccaaataccctgtttgtactcaaaaatagcgtcagtagcggtatgaaaggcggttatcttgcggcctgtggtgtatttattggcgatgcggtattgatgtttctggcatgggctggagtggcgacattaattaagaccaccccgatattattcaacatcgtacgttatcttggtgcgttttatttgctctatctggggagtaaaattctctacgcgaccctgaaaggtaaaaatagcgagaccaaatccgatgagccccaatacggtgccatttttaaacgcgcgttaattttgagcctgactaatccgaaagccattttgttctatgtgtcgtttttcgtacagtttatcgatgttaatgccccacatacgggaatttcattctttattctggcgacgacgctggaactggtgagtttctgctatttgagcttcctgattatttctggggcttttgtcacgcagtacatacgtaccaaaaagaaactggctaaagtgggcaactcactgattggtttgatgttcgtgggtttcgccgcccgactggcgacgctgcaatcctga

SEQ ID NO: 12 - DNA, 2268 bp, Escherichia coli
  misc_feature 1..2268: note = Arginine decarboxylase (Escherichia coli)
  source 1..2268: mol_type = genomic DNA
atgatgaaagtgttaatcgtggaatctgaatttctgcaccaggatacgtgggtcggtaacgctgttgaacgtctggccgatgctttaagccagcaaaatgtgacagttatcaaatccacctcttttgacgatggctttgccattctgtcaagcaatgaagccatcgattgtctgatgttctcgtaccagatggaacaccccgatgagcaccaaaatgttcgtcagctgatcggcaaacttcacgaacgtcaacagaacgtaccggtctttctgttaggcgaccgcgaaaaggccttggcggctatggatcgcgatctgctggagttggtcgacgagtttgcctggattctcgaggatacggcggattttattgccggtcgcgcagtcgccgccatgacgcgctaccgccaacagctgctcccgccgctgttttctgccctgatgaaatactcggacattcacgaatacagctgggcagctcccgggcaccagggcggcgttggcttcacgaaaaccccagctggtcgcttttatcatgactactacggcgagaatttatttcgtaccgacatgggcattgaacgtaccagcctgggctcgctgctggaccacacgggcgcttttggggaatcagagaaatatgcagcacgcgtgttcggtgcggaccgcagttggtccgtcgtggtgggcaccagtggtagcaaccgcaccattatgcaggcgtgcatgaccgataatgatgtggtagtggtggatcgcaattgtcataagagcatcgaacaaggcttgatgctgactggcgctaaaccagtctatatggtgccgtcccgtaatcgctatggtattatcggcccgatttatcctcaggagatgcagccggaaaccctccagaagaaaatctcagagtccccgttaactaaagataaagctgggcaaaaaccgagttattgtgtagtaactaattgtacgtatgatggtgtttgctataacgctaaggaggcccaagatcttctggaaaaaacaagtgatcgtcttcattttgatgaagcttggtacggttatgcgcgtttcaaccctatttacgccgaccactatgcgatgcgtggtgaacctggggatcataatggccctactgtgtttgccacccattctacgcataaactcctgaatgcgttgtcacaggcgagttacatccacgtacgcgaaggccgtggcgctattaattttagccgctttaaccaggcctatatgatgcacgcgacgacaagtccgctgtatgcgatttgcgcgtccaacgatgttgcggtcagcatgatggacggcaacagcggtctgtcgttaacccaggaagtgattgatgaagcggtcgactttcgccaggcgatggcccgtctgtacaaagaattcaccgccgatggctcgtggttcttcaaaccctggaataaagaagtcgtgactgacccgcagacgggcaaaacttatgattttgcagatgccccgacgaagcttcttactacggtccaggattgctgggtgatgcacccgggggagtcttggcatggcttcaaagatatccctgataactggtctatgctcgacccaatcaaagtttcaattttagctccaggcatgggcgaagatggcgaactggaagagacgggggtaccagctgcgttggttaccgcctggttaggccgccatggtattgttccaacacgtaccactgattttcagattatgtttctgttcagtatgggtgtgacgcgcggtaaatgggggacgctggtcaacactctctgctcctttaaacgccattatgatgcgaacacgcccctggcgcaagtcatgccagagctggtggaacaataccctgatacttatgcgaacatgggtatccacgatctgggagatactatgttcgcctggcttaaagaaaataacccgggggcccgcctgaacgaagcatatagtggcctgcccatggcggaaattactccgcgtgaagcctataatgccatcgttgataataacgtcgaattagtatccatcgagaacctccccggtcgtattgcggcaaatagcgtaatcccgtacccgccgggtattcccatgctgctcagcggcgaaaacttcggtgataaaaattccccgcaagtttcttatctgcgcagcctgcaatcgtgggaccatcactttcccgggtttgagcatgaaactgaagggacagagatcatcgatggcatttatcatgtgatgtgcgtcaaggcg

SEQ ID NO: 13 - DNA, 780 bp, Escherichia coli
  misc_feature 1..780: note = ArgT (Escherichia coli)
  source 1..780: mol_type = genomic DNA
atgaaaaaaagcatcctcgcgctgtcactgttagtgggtctcagcgccgcggccagcagctatgctgctcttcctgaaacggtgcgcatcgggacggataccacttatgcaccgtttagcagcaaagatgctaaaggagacttcgtagggtttgatatcgatttaggcaacgagatgtgcaaacgtatgcaagtgaaatgtacctgggtggcttcagactttgatgcattaatcccgagtttgaaagcaaaaaaaattgacgcaattatttcgagcctgagcattacagataagcgccaacaagaaattgccttctcagataaattatatgccgctgattcgcgtcttatcgcggctaaaggctcccctatccaaccaacgttggacagcctgaaggggaaacatgtaggggttctgcaagggtccacgcaggaagcttacgccaatgaaacctggcgttcgaaaggggtcgatgtggtggcgtacgccaatcaggacttggtgtattccgatctggccgcaggtcgtctggacgcagctctgcaggacgaagtggcggcgagtgagggtttcctgaaacagccagcaggcaaagattttgcgttcgccggctcgagtgtaaaggataaaaaatatttcggggatggcacgggtgtcggtttacgcaaagatgatgcagaactgaccgcggcgtttaataaagcccttggcgaactgcgccaagacggcacatatgataaaatggcgaaaaagtactttgacttcaatgtttatggtgat

SEQ ID NO: 14 - DNA, 726 bp, Escherichia coli
  misc_feature 1..726: note = artP (Escherichia coli)
  source 1..726: mol_type = genomic DNA
atgtctattcaattaaatggcatcaactgtttctacggtgcacatcaagccttatttgacatcacgcttgattgcccgcaaggggagacactggtgctgctgggcccgagtggagccggcaaatcgtcgttgctgcgggtgttgaacctgttggagatgccgcgctcaggcaccctgaatatcgcgggcaaccatttcgattttacgaaaacaccgtccgataaagctattcgtgatcttcgtcgcaacgtcggcatggtgtttcagcagtataatttatgtgctcatctgacggttcagcaaaatctgatcgaagcaccgtgtcgtgtgttgggcctgagcaaagaccaagccctggccagcgcagaaaaattattagagcgcctgcgcttgaaaccatattcggatcggtacccacttcacttaagcgggggccagcaacagcgcgttgccatcgctcgtgcgctgatgatggagccgcaagttctcctttttgatgaacctaccgcagcgcttgatccggagatcacggcgcagatcgtcagcatcattcgtgaactcgctgagacgaatattacacaagttattgtgacacatgaggtagaagtggctcgcaagaccgcgtctcgcgtagtgtatatggaaaacggtcatatcgtggagcaaggggacgcctcatgttttacagagccgcagacagaggcattcaaaaattatctgagccac

SEQ ID NO: 15 - DNA, 732 bp, Escherichia coli
  misc_feature 1..732: note = artI (Escherichia coli)
  source 1..732: mol_type = genomic DNA
atgaaaaaagtgcttattgccgccctgattgcgggcttctctctgtctgccaccgcggccgaaaccatccgttttgccactgaagcgtcatatccccctttcgaaagcattgacgccaacaaccaaattgtcggtttcgacgttgacctcgcgcaggccctgtgcaaagaaattgatgccacctgcaccttctctaaccaagcgtttgactcattgattccttcgctgaaatttcgtcgcgtggaagccgtcatgggcggcatggatatcacccccgagcgcgaaaaacaggtcttgtttactacaccgtactacgacaactcggctttgtttgtcggccagcaaggcaagtatacttctgtcgaccagctgaaaggtaaaaaagtccgttcagtccagaacggcaccactcaccagaaattcatcatggacaaacatcctgagatcactaccgtgccgtatgattcttaccagaacgcgaagttagatctggaaaatggtcggattgatggcgtctttggcgacaccgctgtggtacatgaatggctgaaagacaatcctaaattagtggttgtgggagataaggttacggataaggattattttggcaccggtctcggcattgcagtccgccaaggtaataccgaattgcaacagaaattgaataccgcgctggaaaaagtgaaaaaagacggtacatacgaaaccatttacaacaaatggtttcaaaaa

SEQ ID NO: 16 - DNA, 714 bp, Escherichia coli
  misc_feature 1..714: note = artQ (Escherichia coli)
  source 1..714: mol_type = genomic DNA
atgaacgaattcttccctctcgcgtctgcggcaggtatgaccgtgggtttggcggtttgtgcgctgattgtcggtctcgctctggcaatgttctttgccgtatgggagtcagcgaaatggcgtccggtcgcctgggcaggttccgccctggtaaccattctgcgtggtctgccagagatcctggtagttctgtttatctactttggctcttctcagttactgttaacactgtctgacgggtttacgattaacctgggttttgtccagattccggtccagatggatattgaaaatttcgacgtctccccttttctctgtggcgtcatcgcgctgagcttgctctacgctgcatatgcatcacagacccttcgtggtgcattaaaagcggtgccagtaggacagtgggaaagcggccaggcccttggcctgagcaagagcgcaatttttttccgccttgttatgccggccgatgtccgccatgcgttaccaggtctgggtaatcaatggctggtgttgttgaaagacaccgcccttgtctcgctgattagcgtgaacgatttaatgctgcaaaccaaatcgattgcaacccgcactcaggaaccgtttacctggtacatcgtggcggcagcaatctatctggtgatcacacttctgagccagtatattttaaaacgtattgacctgcgtgccacccgctttgagcgccgccctagc

SEQ ID NO: 17 - DNA, 666 bp, Escherichia coli
  misc_feature 1..666: note = artM (Escherichia coli)
  source 1..666: mol_type = genomic DNA
atgtttgaatatctgccggaactgatgaaaggtttgcatactagtctgacgctgaccgtcgcgagtctgatcgttgcgcttatcctggcactgatcttcaccattattctgactctcaagaccccggtcctggtgtggctggtccgcggttacattaccttattcaccgggaccccgctcttggttcgcatttttcttatttactatggtccgggtcagtttccgaccttgcaagaatatcctgcgttatggcacctgctgtctgaaccgtggctgtgcgctctgattgctctgagtgttaactcggcggcctatacgacacagctgttctacggtgctattcgtgcgatcccagaaggtcaatggcagtcttgtagcgcactgggcatgtcaaagaaagatactcttgctattctgctgccgtacgcttttaaacgctctctgagctcgtacagcaatgaagttgtcctggttttcaaaagcactagcttagcgtatacgatcacgctgatggaagtcatgggttatagccagttattatatggtcgcacgtacgacgtcatggtgtttggtgcagcgggcattatctatcttgtagttaatggattactgacgttaatgatgcgcttgatcgaacgcaaagccgtggcattcgagcggcgtaat

SEQ ID NO: 18 - DNA, 729 bp, Escherichia coli
  misc_feature 1..729: note = artJ (Escherichia coli)
  source 1..729: mol_type = genomic DNA
atgaaaaaattggtgcttgcagcactgctggccagtttcactttcggcgcttcggcggccgaaaagattaatttcggtgtcagcgcaacttacccaccgttcgaaagcatcggtgcgaacaatgagattgtaggatttgatatcgatctggccaaagcgttatgcaaacaaatgcaagcggagtgcacttttaccaatcatgcgtttgatagcctgatcccgtcgctgaagttccgtaaatacgacgccgtgatttcggggatggacatcacccctgagcgctcgaaacaggtgagcttcaccactccatattatgaaaactcagcggtggtgattgcgaaaaaagacacctataaaacatttgccgacctgaaagggaaatgtattggtatggagaacggcaccacccatcagaagtatattcaagaccagcacccggaggttaagaccgtaagctacgactcctaccagaatgctttcattgatttaaaaaatggtcgtattgatggtgtattcggagatacagccgtggtgaatgagtggctgaaaaccaatccgcagttgggtgttgcgaccgaaaaagtgacagatccacaatactttgggactggcctgggcatcgcggtgcgcccggataacaaagccctgttggagaaactgaacaacgcgttagctgcgattaaagcggatgggacctatcagaagatttcagaccaatggttcccgcaa

SEQ ID NO: 19 - DNA, 633 bp, Escherichia coli
  misc_feature 1..633: note = ArgO (Escherichia coli)
  source 1..633: mol_type = genomic DNA
atgttctcgtactatttccaaggcttagcactgggtgcggccatgatcttaccgctgggcccacaaaacgcttttgttatgaaccagggaatccgccggcagtaccatatcatgattgcgctgctgtgtgccatctcggatctggtcctgatttgcgccggtatttttggcgggtcggcgttacttatgcaaagcccttggctgctggcgctggtaacgtggggcggcgtagcatttctgctttggtatggattcggcgccttcaaaactgcgatgagttcgaatatcgagcttgcgagtgctgaggtaatgaaacagggccgttggaaaattattgcgaccatgttagccgtgacttggttgaacccgcacgtgtacctggatacttttgtggtgttgggttcactcggtgggcaattagatgtggaaccgaaacgctggtttgccttgggcacaatctcggccagttttttgtggttcttcgggctggcgctgctggccgcgtggctggcaccacgtttacgcaccgccaaggcccagcgcatcatcaacttagtcgtgggctgtgtgatgtggttcattgctctgcaactggcgcgcgatggcattgcgcacgcccaggccctgttctca

SEQ ID NO: 20 - DNA, 1203 bp, Flavobacterium sp.
  misc_feature 1..1203: note = monofunctional lysine-ketoglutarate reductase (Flavobacterium limnosediminis JC2902)
  source 1..1203: mol_type = genomic DNA
atgatgcgctttggcatcattaaagaacgtaagaacccgccagatcgtcgtgtagtgtttacaccgtccgaactgatcaaactgaaagaacagtttccgctggccgaaattaaggtggaatcctcagatattcgcattttttctgatgatgagtatcgtaaacttggatttgaagtaaccgatgacctgagtgattgtgatgtcttgattggcgtgaaagaagtaccgatcgatgccctgctgcccgggaaaaagtatttttttttctctcacacaattaaaaaacagccttacaataaaaaactgctgatcgcctgcttggaaaaaaacatccgtctgattgatcatgagacgatcgtgaatgaagataatcatcgtttgattgggttcggccgttacgcaggtatcgtgggggcctataacggtttccgtgcttttggtattaagtacgagctctttaacctgcccaaagcggaaaccttagcggacaaaacggcacttgtggaacgcctgcgtcggccgatgctgccgccaatcaaaattgtgttgaccggtcacggcaaagtaggtatgggtgcaaaagagattctggatgccatgaaaatcaaacaagtttccgtggaggactacttaacaaaaacctatgacaagccggtgtatacgcagatcgacgttctggactataacaagcggaaagatggcaaaccggcggaacgtgaacacttttatgccaatccgcaggagtatgtctcggacttcgaacgctttaccaaggtgtcggatctgttcatcgcaggccatttctatggcaacggtgcaccggtaattctgactcgcaccatgcttaacgcttctgataataaaattaaagtagttgcggatattagctgtgatgtcggtggccctatcgaatgtacgctgcgcagcagcaccatcgcagagccgttttatggttattatccttccgaaggtaaagaagtcgacgtcaaccatccgggcgcggtggttgtgatggcggtggacaatctgccctgcgagctgcctaaagatgccagcgagggtttcggagaaatgtttctcaaacatgtgattccagccttctacaacaacgataaggacggcattcttgagcgggccaaaatcaccgaaaacggcaaattaacaaaacgcttctcctacttacaggactatgtcgatggtgaa

SEQ ID NO: 21 - DNA, 1356 bp, Flavobacterium sp.
  misc_feature 1..1356: note = saccharopine dehydrogenase (Flavobacterium sp. EM1321)
  source 1..1356: mol_type = genomic DNA
atgcgtaatattttgattatcggcgccggtcggtccgcttcctcgctgattcagtacttattgaataagtcccaagaagaacagctgcatttaaccattgccgatttatcactcgaactggctcagaagaaaaccaataaccatccgaacgctaccgcgctggcgctggatatttataataaggatgaacgtcgtgcggccatcgagaaagcggccattgtgatcagcatgttgccagcgcatctgcatatcgaaatcgcccgggattgcctgtattttaaaaagaaccttgttacggcgagctatattagtgacgcgatgcaggagcttgatgcggaagttaaagagaacaaactgatctttatgaatgaggtcggtttagacccgggtattgatcatatgagcgccatgaaagtcatcgatgaaattcgggaacaaggcggcaaaatgcttctcttcgaaagtttttgcggcggcctggtggcaccagaatcagataacaatttatggaactataaatttacctgggccccacgtaacgtagttctggctggccagggtggtgtggcaaaattcattcaagaaggcacctataaatatatcccgtatgacagcttatttcgccggaccgagtttctggaagtagaaggatacgggcgtttcgaagcttattcgaatcgcgattctctcaaatatcggagtatttatgggctcgatgacgttctcaccctgtttcgtggtacaatccgtcgcgttggcttctccaaagcttggaacatgtttgtgcaactgggcatgacggacgacagctatgttatggaagattctgagaatatgtcctatcgtcaatttattaactcattcctgccttatcacccaaccgatagcgttgaaattaagacccgttttttgttaaaaatcgatcaggatgatatcatgtgggacaaactgctggaactggatcttttcaacgataaaaaaatggttgggttgaaaaatgcgacgccggcacagatcctggagaaaatcctgaacgattcgtggaccctgcaaccggaagataaagatatgatcgtgatgtatcataaatttggttaccagatcaacggcgaaaaagtgcagatggattcacagatggtgtgtatcggccaggaccaaacgtataccgcgatggcaaaaaccgtcggcctgcctgtggcaatggcaactctgctgattctgaacggtaaaatcaaaacaacgggagttcagttgccaatcaataaagaagtttacctgccggtcctggaggaactggagaaatatggcgttgtgttcaaagaacagatgctcccatatcttggatacaaatatagt

SEQ ID NO: 22 - DNA, 1350 bp, Bacillus methanolicus
  misc_feature 1..1350: note = Lysine aminotransferase (Bacillus methanolicus PB1)
  source 1..1350: mol_type = genomic DNA
atgaagaaaaatcattccttgcagtcgcttaaaaaccaagatgagcgtttcatttggcactcgatgaagccgtataaccccgacaagacgatcgttgtcaccaaggccgaaggatcatggattacaacgagtgatggaaagaagtatcttgacgcaatggccggtctttggtgcgttaacgtggggtatggacgcaaagagcttgccgatgccgcgtacgaacagatgatggaaatggcatactatccactgactcagtcacatgtacccgccattcagttagcggagaagttgaacgatctgctggaagacgaatacgtaatcttttttagcaattcggggagtgaggcgaacgaggctgcttttaaaattgctcgtcagtatcatcaacaaaaaggagaccacaatcgctataagattgttgcacgctaccgtgcatatcatgggaactcaattggagccttggcagcgacagggcaggcccagcgtaaatataagtatgagcctctggcctttggattcgtccatgttgcccctcctgactcctaccgtgatgaaactaacgtatccgatccttcgcagttgtccgcagtcaaagaaattgaccgtgtaatgacgtgggagctttcggaaactatcgccgcaatgatcatggaaccgattattactggtggaggcatcttagtgcccccagaggggtatatgaaagcggctaaggaggtttgtgaaaagcacggggctcttttgattgtggacgaggtgatttgcgggtttggtcgtacgggtaagccgttcggattcatgaactatggagtcaagccggacattatcaccatggctaaaggcatcaccagtgcgtatcttccgttgtcagcaactgcagtcaaaaaggaaatctatgatgcctttaaaggtgaggacgaatatgagttcttccgtcatgtcaacactttcggagggtcacccgccgcatgtgcgctggctatcaagaacattcagattttggaggaggaaaagctgtttgaccgctcgggcgacatgggcgaaaaagttttaacagaacttcagaacttgttacgcgatcacccctacgttggcgacgttcgtggaaagggtctgttaatcggaattgaattggttaaagacaagcagacgaaagagcccttaaatacaagcaaagttgacgaagtaatcgctctttgtaaacaggaaggacttctgattggaaaaaatggcatgaccgtggcaggctataacaacgtccttacactgtcccctccgcttaatatcccagagaccgacttagactttttgatcaaagtactgacggcgtccttggagaagattaag

SEQ ID NO: 23 - DNA, 1098 bp, Agrobacterium tumefaciens
  misc_feature 1..1098: note = lysine dehydrogenase (Agrobacterium tumefaciens)
  source 1..1098: mol_type = genomic DNA
atgaaaaatatcgtagtgatcggggcagggaatattggcagcgccattgcgtggatgttggcagctagcggggattatcgcattactgtagcagaccgcagcgcggatcagttagctaatgtaccggctcatgaacgtgtcgacattgttgacattaccgaccgccccgcgctggaagcactgttaaaagggaaatttgcggtacttagcgccgctcccaccgagtttcatttgactgccggaattgcggaagcggccgtcgcggtaggcacgcactacttagacttaacagaagatgtggagtctacccgcaaggtaaaagcgctggctgagacggccgagacagctttaatcccccaatgtgggctggcaccaggttttatttcgattgttgctgccgatttggctgtgaagtttgataaattagattctgttcgtatgcgcgtcggggcgctgcctcagtatcccagtaacgcattgaattacaatttgacttggagcacagatggccttatcaacgagtacattgagccttgcgaggggtttgtagaaggtcgcttgaccgcggtcccggctttagaggaacgcgaggaatttagtcttgatgggatcacctacgaggcattcaacacctcgggcggacttgggaccttgtgcgccacccttgagggtaaggtgcgcacaatgaactaccgtaccatccgctatccgggtcatgtagcaatcatgaaggcacttcttaatgacttgaacctgcgtaatcgccgtgacgttttgaaagatctttttgaaaatgcactgcctggaacgatgcaagatgtcgtaattgtttttgtaacagtgtgtggcactcgcaacggacgctttctgcaagagacttatgccaataaagtgtacgcggggcctgtgtcaggccgcatgatgtccgcgatccagatcacaacagctgctggaatttgcacagtcctggatttgttggccgaaggcgcgcttccgcagaagggcttcgttcgtcaagaggaggtcgcactgcctaagtttttggaaaatcgtttcggacgttattatggttctcacgaaccgcttgctcgtgttggt

SEQ ID NO: 24 - DNA, 1179 bp, unidentified
  misc_feature 1..1179: note = lysine racemase (uncultured bacterium)
  source 1..1179: mol_type = genomic DNA
atggcacatacaggccgtatgtttaagatcgaagccgcggagatcgtagtggctcgcctgccgctgaaatttcgttttgagacatctttcggtgtccagacacataaagtggtgcctttactgatcttacatggcgaaggtgttcaaggggtcgcggaggggacaatggaagctcgccccatgtaccgcgaagaaacgattgccggagcccttgatttgttgcgtggaacttttttacctgcgattctgggccaaacctttgccaatccagaagcggtaagtgatgccctgggctcttaccgcggcaatcgcatggcacgcgctatggtggagatggcagcttgggacttgtgggcccgcacccttggtgtgcctttgggcacactgttgggtggtcacaaggaacaagtcgaggtgggtgtatcgttgggaatccaggcagatgagcaagctacagtagacttagtgcgtcgtcatgttgaacaaggatatcgtcgcattaagttgaagattaagcctgggtgggacgttcaacctgtacgtgcgacccgtgaggcattcatgttaaacacgcttaatgtcggcgcctctggttacgcgggcgcagaactggttacatacgtgaaccgccacccccatatgaacattacggcgttgaccgtatcagcacagtcaaacgatgcagggaagttaatctccgatttgcatccccaattaaagggcatcgttgacttaccattgcagccgatgtccgacatctctgaattcagccccggggtagatgtagtgttcctggctacagctcacgaagtttcacacgacctggccccgcaatttttggaggcgggttgtgtggtctttgatctgtccggcgcttttcgcgttaacgatgctacattttacgagaagtattacggtttcacccaccaatacccagagctgctggaacaggcggcctacgggcttgctgagtggtgtggcaacaaacttaaggaagctaatcttattgcagttcctggatgttaccctaccgccgcacagctggcgctgaagccgttaattgatgctgacctgctggacctgaaccaatggccggtgatcaatgcgaccagtggcgtatctggggcgggtcgtaaagccgcaatttcaaactccttctgcgaggttagcttacaaccg

SEQ ID NO: 25 - DNA, 1407 bp, Bacillus subtilis
  misc_feature 1..1407: note = Lysine transporter yvsh (Bacillus subtilis)
  source 1..1407: mol_type = genomic DNA
atggagcagacgaagaaatggggattttggttactgacggccttcgtcgtgggcaacatggtgggtagtggaatcttttctcttccatcctccctggcgagcatcgcgtcgcctttcggagctacgtccgcttggcttctgacaggtgcgggggtgttaatgatcgccttagtattcggacatttgtccattcgtaaacccgaattgactgccgggcctcaatcatacgcccgtgcattgttcagcgatccaaaaaaggggaatgcggccgggtttactatggtttggggttactgggtcgcgagctggatcagtaacgtagcaatcattacatctctggcggggtatctgaccagcttcttccccatcctggtagacaaacgcgaaatgttttctattgggggtcaagaggtcaccctggggcagctgctgacttttgccgtttgcaccattctgttgtggggcacccatgcgattttggtcgcatcgatcaatggcgcaagcaagctgaattttgtgaccacattatccaaggtcttgggattcgtgtttttcattgtggcagggttattcgtcttccagacgacgctttttggtcatttctatttcccggtccaaggcgagaatggaacgagcatcggtattgggggacaggtgcataacgctgcgatttctacactttgggctttcgtcggaatcgaaagcgccgttatcttgtctggccgcgcgcgcagccagcgcgatgttaaacgtgctaccattaccggacttctgattgcactgtcgatctatattatcgtcacgttaatcacgatgggtgttttaccccacgacaaattagtaggaagtgaaaagccatttgtcgatgttttatatgcaatcgtcgggaacgctggttcagtaatcatggcactgctggccatcttgtgcctttttggaaccatgttggggtggattttactgggctcggaggtgccctaccaagcagccaaagctggtgatttccccgccttctttgccaaaactaataagaaaggttctccagtgattgcgcttatcattaccaatgtcatgtcacaggttttcatttttagcgtgatcagtcgtacaatttccgatgcttttacttttttgactacagcggccacgttggcctatctgattccctacttagtttcagcgatttatagtttgaaagtggttattaaaggcgaaacctatgaccagttgaaaggcagtcgtgtacgtgatggtcttatcgctatcttggcatgtgcatactcagtcttcgtaatcgtgacgggtaccgccgatttgacgacctttattttaggtattgggcttttttttgtgggccttatcgtgtacccatttgtctcgaagaagtttcaaaaggagaagcaggaa

SEQ ID NO: 26 - DNA, 1404 bp, Klebsiella sp.
  misc_feature 1..1404: note = Lysine Transporter LysP (Klebsiella)
  source 1..1404: mol_type = genomic DNA
atgaccatgattgctattggcgggtcgatcggcacagggcttttcgttgcatccggagcaacgattagtcaagcaggtccaggcggggctctgctgtcttatattcttatcggcttaatggtgtattttctgatgacctctcttggagagctggccgcttttatgccagtctccggatcgttcgctacatatgggcaaaactacgtagaggagggtttcgggtttgcgctgggttggaattactggtataattgggctgtgacgatcgcagttgacttggtggcttcgcagcttgtgatgagctattggttccctgacactccgggctggatttggtctgctttgtttttgggcatcatgttcttgcttaactggatctccgttcgcgggttcggtgaagctgagtactggttcagtctgattaaagttgcgaccgttattatcttcatcatcgttggcgtgatgatgattgtcggcattttcaaaggggcgcaaccggctggatggtccaactggggtatcgctgacgccccatttgcggggggcttctcggcgatgattggcgttgccatgattgtcggtttttcctttcagggtacagagttaattggaattgctgctggtgaatccgagaatcctgagaaaaatattccacgtgcggtacgtcaggtattctggcgcattttactgttttatgtttttgcaatcttgattatctcgttgatcatcccttatactgacccatccttattgcgtaacgatgtgaaggatatttccgtgtctcccttcacgttggtatttcagtatgctgggctgcttagtgccgctgcgatcatgaacgcagtcattcttacggctgtactgagcgctggaaactcgggaatgtacgcttcaacacgcatgttatataccttggcatgtgacgggaaagcaccgcgtatctttagcaagctttcccgtggcggtgtgccacgcaatgctctgtatgcaacaactgtaattgctgccttatgctttcttaccagcatgttcggcaaccaaacggtttatctgtggttgctgaacacttcgggaatgacagggttcatcgcctggctgggtattgctatttctcactatcgtttccgtcgcggctacgtgctgcaggggaatgatatcaataatcttccgtatcgttcaggattttttcctcttggacccatttttgcatttgtattgtgtttgattattactcttggccaaaattatgaggcgttcttaaaagatactatcgattggggtggggtagccgcaacctacatcgggattcccttgttccttgttatttggtttggatataagttggctaagggtacccgctttgtccgttattccgaaatgaccttcccagatcgttttaaacgc

SEQ ID NO: 27 - DNA, 630 bp, Pseudomonas sp.
  misc_feature 1..630: note = Lysine Exporter (Pseudomonas)
  source 1..630: mol_type = genomic DNA
atgagtatggaagtctggctggggttttttgcagcgtgttgggtgattagtttgtcaccgggagccggagccatcgcctctatgtcatcgggtttacaatatggcttctggcgtggctactggaatgcacttggattgcagcttggtttaattatgcaaattgcaattatcgctgcgggcgtcggagccgtcttggcggcctcggctacggccttccaggtaattaaatggttcggagttgggtatcttgtgtatttagcatacaaacaatggcgtgcactgcccatggatatgtcggatgaaagcggggtgcgtccaatcggcaaaccattatcgctggtatttcgtggatttttggtgaatatctccaacccaaaagctttagtattcatgttggccgttttaccccagttcctgaatccccacgcccccttgttaccccaatacgtggctatcactgtgacaatggttacagttgacttgttagtgatggccggatacacaggtttagcatctcatgtattacgtatgcttcgtaccccaaaacagcaaaaacgcctgaaccgcaccttcgccggtttattcatcggagcggccacattccttgccactttgcgccgcgcaccagta

SEQ ID NO: 28 - DNA, 1014 bp, Escherichia coli
  misc_feature 1..1014: note = Asparaginase (Escherichia coli)
  source 1..1014: mol_type = genomic DNA
atgcagaagaaatcgatctacgtcgcgtacacgggcggcaccattgggatgcagcgttcggagcagggttacatccccgtttccggtcacttgcagcgccagctggccttgatgcccgagttccatcgccccgagatgccagattttaccattcatgagtacactccacttatggattcatcggacatgacgccggaagactggcaacacattgcagaagatatcaaggctcactatgatgattatgacggctttgttattttacacggtactgacacaatggcatacacagcttctgcactttcctttatgcttgagaaccttggtaagcccgtgatcgtgaccgggtcgcagatcccccttgccgaattgcgcagtgacgggcagatcaatcttcttaatgcgttatatgtggccgctaactatccgatcaatgaagtgactttattcttcaataaccgcttgtaccgtggaaaccgcactacgaaagcccatgctgatggctttgacgcctttgcatccccaaatctgcctccccttttggaagccgggattcacatccgtcgtttaaatacaccccccgccccacatggagagggggagcttatcgtacatccaattacccctcaacctatcggagttgtaacgatttaccctggtattagtgccgacgtagtccgcaatttccttcgccagcccgtgaaagcattgatcttacgttcctacggtgtagggaacgcgccacagaataaggcatttctgcaagaattacaagaggcatcggatcgtggtatcgtggtagtcaacctgacacagtgcatgtcaggtaaagttaatatgggtggatacgcaaccgggaatgcattagctcatgcaggggtaattggaggcgctgatatgacggtcgaagctaccctgacgaagcttcattatctgttatcccaggagttggacaccgagaccattcgcaaagctatgtctcagaaccttcgcggtgagcttactcccgatgac

SEQ ID NO: 29 - DNA, 1461 bp, Mycobacterium bovis
  misc_feature 1..1461: note = Asparagine transporter ansp2 (Mycobacterium bovis)
  source 1..1461: mol_type = genomic DNA
atgccgcctctggacatcaccgacgaacgcttgactcgcgaagatacaggatatcacaaaggccttcactcccgtcagcttcagatgatcgctcttggaggtgctattgggaccggactttttctgggggcaggcggacgtctggcttctgccggaccgggattattcttggtttatggtatctgtggcatttttgtctttcttattcttcgtgccttgggagaacttgtgcttcaccgccctagttcaggatcatttgtatcctacgcgggggaattttatggtgaaaaggtcgcgttcgtcgcggggtggatgtattttttgaattgggcaatgactgggattgtggacactacagccatcgcccactattgccactattggcgcgcttttcaaccaattccacagtggacgttggcccttattgcgttgttagttgtattatccatgaatctgatctccgtccgcttattcggggaacttgagttttgggcctcgcttattaaagtaattgcgcttgttacgttcctgattgtagggactgtattcctggcggggcgttacaagattgacgggcaagaaactggtgtatcattatggtcatctcatggcggaatcgttcctacggggttactgcccattgtccttgtgacctctggagttgtgttcgcatacgcagccatcgagctggtaggaatcgcagccggggagacggccgaaccagccaaaatcatgccccgcgcaatcaattcggtcgtccttcgtattgcgtgtttttatgtgggatctacggtgcttctggcgttgcttttaccatacacggcttataaggagcacgtaagtcccttcgtaacatttttcagcaaaattggaattgatgccgcggggagtgtaatgaacttggtagtgcttacggcagcgttatctagtttgaacgctggtttgtattccacaggacgcatcctgcgctcaatggcgatcaacggcagcggaccacgctttacggcacccatgagtaaaaccggtgttccttatggcggtatcttgcttacagcaggtatcggtttattgggaatcattcttaatgcgatcaaaccctcgcaggcgttcgaaatcgttttacacatcgctgctaccggcgtaatcgcagcctgggctacgatcgtggcttgtcagttgcgcttacatcgcatggccaacgctggccaacttcagcgccctaagttccgtatgcccttgtcaccttttagcgggtacctgactttggcgtttctggcgggcgtgctgattctgatgtatttcgatgagcagcacgggccctggatgatcgccgcgacagtaattggggttcctgcccttattgggggttggtacttggttcgtaaccgtgtgactgccgtcgctcatcacgctattgaccacactaagagtgtagctgtggttcattcggcagatcccatt

SEQ ID NO: 30 - DNA, 1344 bp, Bacillus subtilis
  misc_feature 1..1344: note = Serine ammonia lyase (Bacillus subtilis)
  source 1..1344: mol_type = genomic DNA
atgtccattaatcaggaggcgcttcacgtactgttgaaggatccttttattcatcgtcttattgatgctgagccagtgttttgggcaaatccaggtatgaaggaggggctgctttttcacgctgacgagtgggaaagtgagattgccgaagcagagaaacgcttgcgtcgttttgcgccttatatcgcggaggttttcccagagaccaaagatgcaaagggcatgatcgagtctccactgtttgagatgcaacatatgaaaaagaaactggaggcagcataccaacaacctttccccggacgttggctgcttaagtgtgaccatgaacttcccatttccgggtcgattaaggcccgcggaggtatctatgaagtcttgaaacatgccgaaaagctggctcttcaggaggggatgcttcaagagtcggacgattatcgtatgttgcaagaagatcgtttcgcggccttcttcagccgctattctatcgcagtgggctccacgggcaatttaggtttaagtatcgggattattggcgctgctcttggtttccgcgtaacagttcacatgagtgctgacgctaagcaatggaagaaagatctgttacgtcagaaaggggtaaccgtaatggaatatgagagcgattattcagaagcagttaaagaaggtcgtcgccaagcagaacaagacccattctgttacttcattgatgatgaacatagccgtcaattgttcctgggctacgcagttgccgcgtcccgccttaagacacaactggattgcatggaaatccaacctggtcccgaaacacccctgttcgtgtatcttccctgtggcgtaggtggggggccaggaggtgtcgctttcgggttgaaactgctgtatggagatcacgtccatgtatttttcggagaaccgacgcaatccccgtgcatgcttttaggcttatattctggcttacacgagcagatttcagttcaagacattggattggacaaccgtaccgcggcggatggcttggcggtagggcgtccctcaggattcgtaggaaaattaatcgaaccactgctgtcgggctgctatactgtagaagacgatacactgtatgctttactgcacatgctggcagcttcggaatccaagtatcttgaacctagcgccttggcggggatgttcggcccgatccagctgttcagcacagaagaaggacgtcgctattctcagaaacataaaatggagcatgcggtgcacgttatctgggggacggggggtagcatggtgccaaaggaggagatggccgcatacaaccgcatcggggcggatctgttaaaaaatgaaatgaagaag

SEQ ID NO: 31 - DNA, 1374 bp, Pseudomonas fluorescens
  misc_feature 1..1374: note = SdaA (Pseudomonas fluorescens F113)
  source 1..1374: mol_type = genomic DNA
atgtccctttcagtgtttgatctttttaagatcggaattggtccctcgtcctctcataccgtaggacctatgcgtgcggccgctcgttttgccgaaggtctgcgccgcgacgacctgctgaactgtactactagcgtgaaagtcgagctgtacggatctctgggcgcgactggtaaagggcacggttcggacaaagcagtgttactgggattggagggagaacaccctgacactgtcgacaccgagacggttgacgctcgtttacaggcgatccgcagttcaggccgcctgaatttattgggggagcatagcattgagtttaatgaaaagctgcacttggcaatgattcgcaagccgttagctttccatccgaatggcatgattttccgtgcgtttgatgctgcgggcttacaggtacgttcccgtgagtattactccgtcggcggagggttcgttgtagacgaggacgcagcgggtgccgaccgtatcgtcgaggatgcaacacctttgacattccccttcaagagcgcgaaggatcttttaggtcattgttctacttatggtttaagcatcagccaagtcatgcttacaaacgagtctgcgtggcgtccggaagcggagacccgcgcagggcttcttaaaatttggcaggtgatgcaagactgcgttgccgcggggtgtcgcaatgagggcatccttccaggaggtcttaaagtaaagcgccgcgcggctgcgttgcatcgtcaattgtgtaagaaccccgaggctgccctgcgcgatccgttaagtgtattagattgggtgaatttgtatgcgttagcggtaaatgaagagaacgcctacggtggacgcgtggtcacggcgcccactaatggagccgcaggaatcattcctgccgtattgcattactacatgcgctttattccgggggcatctgaggacggagtagtccgcttccttcttacagcggcggcaatcgggatcttgtataaagagaacgcctctattagtggggctgaggttggctgtcagggcgaagtaggagtggcatgctccatggcagcgggggcgttgtgcgaagtcttgggaggctcggtccaacaagtagaaaacgcagcagaaatcggaatggagcataaccttggcttgacatgtgatcctatcggcgggttagtacaggtcccgtgtatcgagcgtaacgcaatgggatctgttaaagccattaacgcagtacgcatggctatgcgcggggacggtcaccatttcgtctcccttgacaaagtaattcgtaccatgcgtcaaactggggccgacatgaaaagcaagtacaaggaaaccgcgcgtggtggacttgctgtcaacatcatcgagtgt

SEQ ID NO: 32 - DNA, 1365 bp, Klebsiella pneumoniae
  misc_feature 1..1365: note = sdaB(Klebsiella pneumoniae)
  source 1..1365: mol_type = genomic DNA
atgattagtgtgtttgacatctttaaaatcggtatcggtccgtcttcttcccatacggttggtcccatgaaagcagggaagcagtttaccgacgacttaattgctcgtggactgctggcagaggtcagtaaggtcgtggttgatgtttatggctccctttcattgacgggcaaaggtcaccatactgacattgctatcattatgggtctggcgggaaacttgccagacaccgttgacatcgacgccatccccggcttcatccaagatgttaacactcacggacgtctgatgttagcgaatgggcagcatgaagttgatttcccggtagaccagtgtatgaattttcacgctgacaacctgtccttgcacgagaatggaatgcgtattacggctcttgcgggagacaaagtgttgtactctcagacttactactcaatcggcggcggattcattgttgatgaggaacattttggccaaacaacggaggctcctgtagccgtcccatatccatacaaaaacgccgctgatttgcagcgtcattgccgtgaaactggtttgagtttatctggacttatgatgcaaaacgaacttgcattgcatagcaaagaagctctggaacagcactttgctgcagtttgggaggttatgtctgccggcattgagcgcggcattacaactgaaggtgtgttgcctggcaaattacgtgtaccccgccgcgccgcggcactgcgtcgtatgttagtctcgcaagacacgacgaactcggaccctatggctgttgtagattggatcaatatgttcgcgttggccgtcaacgaggagaacgcggcgggcggtcgcgttgttacagcccccacaaatggcgcgtgcggaattgttccggccgtgctggcatattatgacaaatttatccgcaaagtcaactccaacagtctggcgcgttatatgctggtggcaagtgcaatcggctcactttataagatgaatgcgagcatctccggcgcagaagttggctgccaaggtgaagtgggggtcgcctgctctatggcagcggctggcttggcagagctgttgggcgggtcgccagggcaagtgtgcattgcggctgaaattgcgatggagcataacttgggccttacgtgcgatcccgtagctggccaagtgcaggtaccgtgtatcgaacgcaatgcaattgcagccgtaaaagcagtaaatgcggctcgcatggccttacgtcgtacttccgagccccgtgtgtgcttggataaggtgatcgaaaccatgtatgagacaggtaaggacatgaatgcaaagtatcgtgaaacgtctcgtggaggcctggccatgaagatcgtcgcgtgtgac

SEQ ID NO: 33 - DNA, 1362 bp, Escherichia coli
  misc_feature 1..1362: note = tdcG L-serine dehydratase (Escherichia coli O157:H7 str. SS17)
  source 1..1362: mol_type = genomic DNA
atgattagtgcattcgatattttcaagattggaatcggcccctcgtcatcgcacacggtgggcccaatgaacgcaggtaagtccttcattgatcgccttgagtcgagtggcttattgacagcgacaagccacattgtcgtggacctgtacgggagtctgtcgttgacgggcaaaggccatgcgaccgatgttgctattatcatgggattggccgggaattcaccgcaggacgtagtaatcgatgaaatcccggccttcattgagctggtaactcgttcgggccgtctgccagtcgcaagcggagctcatatcgttgacttcccagttgccaagaacattatttttcaccctgaaatgttacctcgccatgagaacggaatgcgtatcacagcatggaaagctcaggaagaattattgagtaagacgtattactcggttggtggcgggttcatcgtcgaggaagagcacttcggtttatctcatgacgtagaaacaccagtaccatacgacttccattcagcaggtgagttgttgaaaatgtgcgattacaatggccttagtatttcgggacttatgatgcataacgaattagcgcttcgttcgaaggccgaaattgacgccggcttcgcacgtatctggcaagttatgcatgatggcatcgaacgtggtatgaacaccgaaggtgtgttaccaggacccttgaatgttccgcgtcgtgcagtcgcactgcgtcgtcaacttgttagtagtgacaacatttccaatgatccaatgaacgtgattgactggatcaacatgtacgcgctggcggtctcggaggaaaacgccgctgggggtcgcgtggtaacagcacctacgaatggggcttgcgggatcatccctgcggtattggcctattacgataagtttcgccgtccagtcaatgagcgctcaatcgctcgttacttcctggcggcgggggctatcggcgctttatacaagatgaacgcctctatttcaggggcggaggtcggttgtcaaggagagattggggtcgcgtgctctatggcagctgcaggtttgacagaattattaggcggcagcccagcccaagtttgcaacgcggctgaaatcgcaatggaacataatcttggtctgacctgtgaccctgtcgcaggtcaggtacagattccttgcattgagcgtaatgcaatcaacgcagtaaaagctgttaatgcggcgcgtatggctatgcgtcgcacatcagccccgcgtgtgagcctggataaggtaatcgagaccatgtacgaaaccggtaaagacatgaatgacaaataccgcgaaacctctcgcgggggtcttgcaattaaagtcgtgtgtggc

SEQ ID NO: 34 - DNA, 1251 bp, Escherichia coli
  misc_feature 1..1251: note = glyA(Escherichia coliEPEC C342-62)
  source 1..1251: mol_type = genomic DNA
atgttgaaacgtgagatgaatattgccgactatgatgcagaattatggcaagctatggaacaagagaaagtccgccaggaagaacatattgaattaatcgcctctgaaaattacactagtccccgcgttatgcaagcccaaggcagccaattaactaacaaatatgccgagggatatcctgggaaacgctactatggaggttgcgagtatgtagatattgtcgaacagttagcaatcgaccgcgcgaaagagcttttcggcgcagactatgcaaacgtgcagccccattcgggtagccaagcgaattttgcggtctataccgcactgctggaaccgggagacacggtactgggtatgaatttagctcatggtggtcacttaacgcacgggtcccccgttaatttctctggaaaactgtacaacatcgtcccctatggaatcgatgctaccggccacattgattacgcggatcttgagaagcaagctaaggaacataaaccaaagatgatcattggcggtttttcagcttatagtggtgtcgtcgactgggctaagatgcgtgaaattgcagactctattggcgcgtacctttttgtcgacatggcccacgtggctggcttggtggcggcaggggtctacccgaaccccgttccccatgcgcatgtcgtgaccaccacgacacataagacactggctgggcctcgtggtggcttaatcttggccaagggggggtctgaggaattatacaaaaaacttaactcagccgtttttccaggcggacagggtggtccgttgatgcacgtgattgctggaaaggcggtcgctcttaaggaagccatggaacctgaattcaaaacgtaccaacagcaggttgcaaaaaacgccaaagcgatggttgaggttttcctggaacgtggttacaaagtcgttagtgggggtaccgataatcatcttttcttagttgacctggtagataaaaatttgaccggaaaggaggcggacgctgccttaggccgtgcgaatattaccgtcaataaaaactcggtgccaaatgatcccaagtcgcctttcgtgacttcaggaatccgcgtaggaactcccgcaattacacgccgcgggttcaaggaagctgaggcgaaggagttagcaggatggatgtgtgatgttttagactcgattaacgatgaggcggtgatcgaacgtatcaaaggtaaagtattagatatttgcgcccgttatccagtttatgcc

SEQ ID NO: 35 - DNA, 1287 bp, Escherichia coli
  misc_feature 1..1287: note = SdaCserine STP transporter (Escherichia coliBL21(DE3)
  source 1..1287: mol_type = genomic DNA
atggagaccacgcagacttctacaattgcgagcaaagatagccgttctgcttggcgcaaaactgatactatgtggatgttgggcctgtatggaacagctattggggccggggtactgtttttgccaatcaatgctggagtggggggtatgatcccgctgatcattatggcgattcttgctttcccaatgacattttttgcacatcgcggtcttacacgctttgtcctttcaggaaagaatcctggggaggacattacggaggttgtagaagaacattttggcattggggctgggaaacttatcacattgctgtatttttttgcaatctatcccattttgcttgtctatagcgtagcaatcacgaacaccgtagaatcattcatgtcgcaccagttaggcatgacacctccgccacgtgcgattctgtcattgatcttgatcgtgggaatgatgacaattgttcgtttcggagagcaaatgatcgtgaaagccatgtcaattttggtatttccgttcgtgggagtcttaatgttgctggcattgtatttaattccccagtggaatggtgccgctctggagaccttgtcgttggatacggcgtcagcgaccggtaatggtctttggatgacgctttggttggccattccggtcatggttttttcatttaaccactcaccgatcattagctcgttcgctgtggcgaaacgcgaagaatacggtgatatggctgaacaaaagtgctcgaagattttggcattcgcccacatcatgatggtacttacggtcatgttcttcgtgttttcttgcgtccttagtttaaccccagcggacctggcggctgcaaaggaacaaaatatcagcatcttaagctatttggcgaatcatttcaacgcgcctgttatcgcatggatggcacccattatcgctatcattgcaattaccaaatctttcttagggcactacttgggtgcgcgcgaaggatttaacgggatggttatcaagtcgcttcgtgggaaaggaaagagtatcgagatcaataaacttaatcgcatcaccgccttgttcatgttagtaacaacgtggatcgtcgctacacttaatccctccattctggggatgattgaaacgcttgggggtccaatcatcgcaatgatcttgtttctgatgccgatgtacgctatccagaaggtacccgcaatgcgtaaatactctgggcatatctccaacgtgtttgttgttgttatgggattaatcgctatttctgctatcttctatagtctgttctcc

SEQ ID NO: 36 - DNA, 438 bp, Lactobacillus saniviri
  misc_feature 1..438: note = threonine Serine Exporter (Lactobacillus saniviri JCM 17471 = DSM 24301)
  source 1..438: mol_type = genomic DNA
atggcgtattctgtccagttcctgatccaactgtccttctcgtaccttgccactgtggcttttgctatctgcatcaacgttccacgtcgtgcgttaaattttgccggatgggccggtgccatcgggtggatctgctactggctgctgaacacacatggcacgggccgcatgttcgctaacctgattggcgctgtcgcagttggggtatgtggtatcattttcgctcgcatcaagaagatgcccgtgattattttcaatattccggggctggtgccattagtgcctggagcaaccgcctaccaggcagttcgcgctcttgcgttgggaaatatggaccttgctatccagcttggagttcgtgttattatggtcgcaggggcaatcgcggtgggattcatggttagtcagcttctgtcagagttgacttaccgcttgcac

SEQ ID NO: 37 - DNA, 930 bp, Escherichia coli
  misc_feature 1..930: note = glutaminase YbaS (Escherichia coli ST131)
  source 1..930: mol_type = genomic DNA
atgctggatgctaataagctgcagcaggctgtcgatcaggcttatactcaatttcattctttgaatggtgggcagaatgccgattacattcctttcttggctaatgtcccagggcaattagcagccgtagctattgtaacatccgatggcaacgtgtattctgccggggactcggactaccgcttcgcacttgagtctatcagtaaagtctgcactttggcactggcgctggaggacgttgggcctcaggccgtgcaggacaaggttggggctgatcctacagggctgccattcaactcagtaattgctttggaattacacggtggaaaaccactgtcaccgctggtgaacgcgggggcaatcgctaccacgtctttgattaatgcagaaaatacggaacagcgttggcaacgtattttgcatattcagcagcagcttgctggtgagcaagtcgcactttctgatgaagtgaaccaaagtgaacaaactactaattttcacaaccgtgcaattgcttggttactgtacagtgctggctacttgtactgtgacgcaatggaagcctgtgatgtttatacacgtcagtgcagtactttgatcaacacaatcgaattggcaacattgggagctacgttagccgctgggggcgtgaatccgttgacacataaacgcgttctgcaagcggacaatgtgccctatattttggctgaaatgatgatggaagggctttatggccgctctggggactgggcctaccgtgtaggcttgccaggaaagtcgggggtcggaggagggattctggccgtggtgcccggcgtaatgggaattgccgcgttttcgcctcccttagacgaagaaggtaacagcgtgcgcggacaaaagatggttgcgagcgttgcaaagcagcttgggtataacgtatttaaaggg

SEQ ID NO: 38 - DNA, 924 bp, Escherichia coli
  misc_feature 1..924: note = Glutaminase (Escherichia coli O145:H28 str. RM12581)
  source 1..924: mol_type = genomic DNA
atggccgtcgcaatggataacgccattttagagaatatcctgcgccaagtgcgcccattaatcggacaaggcaaggttgcggattacattccggccttagctacagtggatgggagtcgcctgggaatcgctatttgcactgttgacggccaattgtttcaggcaggcgacgcacaagagcgcttctccatccagagcatttctaaagtgttgtcattggttgttgctatgcgtcactactctgaggaggaaatttggcagcgcgtggggaaggacccgtccggcagtccatttaattcgttggtacagttggagatggaacaaggaatccctcgtaatcccttcatcaatgcaggtgctcttgtagtctgcgacatgttacaaggtcgtttatctgcccctcgccaacgcatgttggaagttgtgcgtggtttgtctggagttagcgatatcagctacgacacggtcgtggctcgcagtgaatttgaacactcagcacgcaatgcagcgattgcgtggttaatgaagtcgtttgggaattttcatcacgatgtgacgacagtccttcaaaattatttccactactgcgcattgaagatgtcgtgcgtagagcttgcccgtacgttcgtctttcttgcgaaccagggcaaggccatccatatcgacgagcccgtcgtaaccccgatgcaggcgcgtcaaatcaatgcgctgatggcgacatcgggaatgtatcagaatgcgggggagttcgcctggcgtgtcggattaccagctaaatccggtgtaggcggtggaatcgttgccattgtgccccatgaaatggctatcgctgtgtggtccccagaattagatgacgcaggaaattcgttagcaggtattgcggttttagaacaacttacgaaacaattaggacgctcggtgtat

SEQ ID NO: 39 - DNA, 927 bp, Bacillus subtilis
  misc_feature 1..927: note = ylaM (Bacillus subtilis subsp. subtilis str. 168)
  source 1..927: mol_type = genomic DNA
atggtgtgtcagcataatgatgaattagaggctcttgtcaagaaggcaaaaaaggttacggataagggggaggtggctagttacattccagctctggctaaggcggacaaacacgacttaagtgtcgcaatctactatagcaataatgtgtgcctgtccgcaggggacgttgaaaagacgttcactctgcaatccatcagcaaagttctgtcgttagctctggtacttatggagtatgggaaggataaggtattcagttatgttgggcaggaacctacaggtgatccctttaacagcatcattaaactggagacagtcaacccctctaagccattaaatccgatgatcaatgcgggcgcgttagtagtgaccagtcttatccgcggacgtacggtgaaggagcgtcttgactatcttcttagctttatccgtcgtctgactaataatcaagaaattacatactgccgcgaggtagcggaaagcgaatattctacttcaatgattaaccgtgcgatgtgctattatatgaaacagtatggaattttcgaagatgacgttgaagcggttatggacctttatacaaagcaatgcgctattgaaatgaactcacttgatttggctaagatcggttcggttttcgccttgaacggacgccatcctgaaaccggggagcaagtgatttcgaaggatgtagcccgtatctgtaagacgtttatggtgacgtgtggaatgtataatgcctctggtgaatttgcgatcaaagttggtatccctgcgaaatcgggagtgtcaggtgggattatgggtatctccccttacgatttcggaatcgggatctttggacccgcgctggacgagaaggggaatagtattgctggtgtgaagcttttagaaatcatgagcgagatgtaccgtcttagtatcttt

SEQ ID NO: 40 - DNA, 981 bp, Bacillus subtilis
  misc_feature 1..981: note = ybgJ(Bacillus subtilis)
  source 1..981: mol_type = genomic DNA
atgaaagagttgattaaagagcatcaaaaggatatcaatcctgcattacaactgcatgactgggtagaatactaccgtccatttgcggcaaatggccaaagtgcaaactatatccccgctttagggaaggtgaacgacagccagttagggatctgcgtactggaaccggatggcaccatgattcacgctggggattggaatgtgtcctttaccatgcagtcgatttcaaaagtaattagcttcattgctgcctgcatgtcgcgtggaatcccgtatgtcttggatcgtgtagacgtggaacccacaggagatgcttttaatagtatcatccgtttagagatcaacaaaccaggaaagcctttcaatcctatgattaatgccggagctttgactatcgctagcattcttccaggagagtccgcttacgaaaaacttgagtttttgtatagcgtgatggagactttaatcggtaaacgcccccgtattcacgaagaagtattccgttctgaatgggagaccgctcatcgcaatcgcgccttagcctactatcttaaagaaacaaacttcttagaggccgaggtcgaagagacactggaagtatatttgaaacaatgcgcgatggaatcgaccacggaagacatcgccctgatcgggttgatcctggcccacgatgggtatcatcctatccgtcatgagcaggtcattcccaaggatgttgccaagttggctaaagcgttaatgttgacctgtggcatgtataacgcttctggaaagtatgcggctttcgttggagtacccgcaaaatctggagtttcgggtggtattatggccttggtgcctccaagtgcgcgtcgcgaacagccgttccagagcgggtgcggtatcgggatttatggacctgcaattgatgagtacgggaatagcctgacgggcggcatgcttttaaaacacatggcccaagagtgggaactgagtattttc

SEQ ID NO: 41 - DNA, 3436 bp, Escherichia coli
  misc_feature 1..3436: note = Glutamine permease glnHPQ operon (Escherichia coli)
  source 1..3436: mol_type = genomic DNA
ccatggcagaacgtgcagtgcagctgggcggtgtagctctggggaccactcaagttatcaacagcaaaaccccgctgaaaagttacccgctggacatccacaacgttcaggatcacctgaaagaactggctgaccgttacgcaatcgtcgctaatgacgtacgcaaagcgattggcgaagcgaaagatgacgacaccgcagatatcctgaccgccgcgtctcgcgacctggataaattcctgtggtttatcgagtctaacatcgaataaatccatcgctgatggtgcagaactttagtacccgataaaagcggcttcctgacaggaggccgttttgttttgcagcccacctcaacgcacttatttagtgcatccatctgctatctccagctgattaagtaaattttttgtatccacatcatcacacaatcgttacataaagattgttttttcatcaggttttacgctaaataatcactgtgttgagtgcacaattttagcgcaccagattggtgccccagaatggtgcatcttcagggtattgccctataaatcgtgcatcacgtttttgccgcatctcgaaaaatcaaggagttgcaaaactggcacgattttttcatatatgtgaatgtcacgcaggggatcgtcccgtggatagaaaaaaggaaatgctatgaagtctgtattaaaagtttcactggctgcactgaccctggcttttgcggtttcttctcatgccgcggataaaaaattagttgtcgcgacggataccgccttcgttccgtttgaatttaaacagggcgataaatatgtgggctttgacgttgatctgtgggctgccatcgctaaagagctgaagctggattacgaactgaagccgatggatttcagtgggatcattccggcactgcaaaccaaaaacgtcgatctggcgctggcgggcattaccatcaccgacgagcgtaaaaaagcgatcgatttctctgacggctactacaaaagcggcctgttagtgatggtgaaagctaacaataacgatgtgaaaagcgtgaaagatctcgacgggaaagtggttgctgtgaagagcggtactggctccgttgattacgcgaaagcaaacatcaaaactaaagatctgcgtcagttcccgaacatcgataacgcctatatggaactgggcaccaaccgcgcagacgccgttctgcacgatacgccaaacattctgtacttcatcaaaaccgccggtaacggtcagttcaaagcggtaggtgactctctggaagcgcagcaatacggtattgcgttcccgaaaggtagcgacgagctgcgtgacaaagtcaacggcgcgttgaaaaccctgcgcgagaacggaacttacaacgaaatctacaaaaaatggttcggtactgaaccgaaataataacgctacacctgtaaaacgcactggcagttccctctcccctatggggagaggattagggtgaggggcgcaaacccgctccggggccattaattaccctgaatttgattatttacaccacggtaacaggaacaacatatgcagtttgactggagtgccatctggcctgccattccgcttctgattgaaggtgccaaaatgaccctgtggatttcggtcctcggtctggcaggcggtctggtaatcggattgctggcaggttttgcacgcaccttcggaggttggatagccaaccacgtcgcgctggtctttattgaagtgatccgcggcacacctatcgtcgtccaggtgatgtttatttatttcgccctgccgatggcgtttaacgacttacgcatcgacccatttactgcggcggtggtcaccatcatgatcaactccggcgcgtatattgcggaaatcacgcgtggtgcggtgctgtctatccacaaaggttttcgtgaagcaggactggcgctcggtctttcacgttgggaaaccattcgctacgtcattttaccgctggcactgcgtcgtatgctgccgccgctgggtaaccagtggatcatcagcattaaagacacctcgctgtttattgtgatcggcgtggcggaactgacccgtcaggggcaagaaattattgccggtaacttccgcgcccttgagatctggagcgccgtggcggtgttctatctgattattaccctggtgctgagctttattctgcgtcgtctggaaagaaggatgaaaatcctgtgattgaatttaaaaacgtctccaagcactttggcccaacccaggtgctgcacaatatcgatttgaacattgcccagggcgaagtcgtggtgattatcgggccgtccggttccggtaaatcgaccctgctgcgctgcatcaacaaactggaagaaatcacctccggcgatctgattgtcgatggcctgaaggttaacgatccgaaagttgacgagcgcctgattcgccaggaagcaggtatggtgttccagcagttttacctcttcccgcatctgacagcgctggaaaacgtcatgtttggcccgctacgcgtgcgtggcgcgaacaaagaagaggcggaaaaactggcacgtgagctgctggcgaaagtcggtctggcagaacgtgcacatcactacccttccgaactttctggtggtcaacagcagcgtgtggcgattgcccgcgcgctggcggtgaagccgaaaatgatgctgtttgatgaaccgacttccgctcttgacccggaactgcgccatgaagtgctgaaggttatgcaggatctggctgaagaagggatgacgatggtgatcgtgacccacgaaatcggttttgccgagaaagtagcttcgcggctgatctttatcgacaaaggccggattgcggaagatggcaatccgcaggtgttgatcaagaacccgccgagccagcgcttgcaggaatttttgcagcacgtctcttaataagacacattgcctgatcgtacgcttatcaggcctacaggatatctggcaacttattaaaattgcatgaacttgtaggacggataaggcgttcacgcgcatccggcaaaaaagcccgcacgttgtcagcaacctgcttaatatcccttcctccctttcacccgaaagggaggcacaccagattcctctcatttaaaatcgcccctcctccagcatctatacttatctttttgctctattttctcactggaggagtcatgcggtggatcctgttcatcctcttctgcctgctgggcgcacctgcccacgcggtatccatacccggcgttacaaccacaacgacaacggactcaacgactgaaccggccccggaaccggatatcgaacaaaaaaaagcggcctatgcgcactggcggatgtgctggataatgacacctcgcgtaaagagttgatcgaccagttgcgcaccgttgccgctacgcccctgctgaaccggtacc

SEQ ID NO: 42 - DNA, 744 bp, Escherichia coli
  misc_feature 1..744: note = Glutamine permease H glnH (Escherichia coliEPEC C342-62)
  source 1..744: mol_type = genomic DNA
atgaaaagtgtacttaaagtgtcattggcagcactgacacttgcatttgcagtctccagtcatgctgcggacaaaaagttagtcgtagcgactgacactgcgtttgttcctttcgaattcaagcagggggacaagtacgtcggctttgacgtagacctttgggccgccattgcaaaagagcttaagttggattacgagttaaagcctatggacttcagtggtatcattcccgccctgcaaacgaaaaacgtggatcttgcgcttgcaggcattactattaccgacgaacgcaagaaggcgattgacttcagcgacggctattataagtcgggtcttttagttatggtaaaagccaacaataatgatgtgaaaagcgtgaaagatttggacgggaaagtagtggcagttaaatcaggtacagggagtgtggattacgcgaaagctaatatcaaaaccaaagacttacgtcaattcccgaatatcgacaatgcgtatatggaactggggacgaaccgtgcggatgcggtgctgcacgatacacccaacatcctttatttcattaaaacagctggtaatggtcaatttaaagctgtaggcgacagcctggaagcccagcaatacgggatcgcgttccctaagggctctgatgagcttcgtgacaaggtaaacggggcgcttaaaacgctgcgtgaaaacggaacgtacaatgaaatctataagaagtggttcggaaccgagcccaaa

SEQ ID NO: 43 - DNA, 657 bp, Escherichia coli
  misc_feature 1..657: note = Glutamine permease P glnP (Escherichia coliB354)
  source 1..657: mol_type = genomic DNA
atgcaattcgattggagtgcgatttggcctgccattccccttctgattgagggtgcaaaaatgactctgtggatttcagtgctggggttagccggaggtcttgttattgggttattagcagggtttgcacgcactttcgggggatggattgcaaatcatgttgcgctggtcttcatcgaagtcattcgtggcacccccatcgtggtccaagtgatgtttatttacttcgcgttgccaatggcatttaacgatcttcgtattgatccatttactgcggcagtggtgactatcatgattaatagtggggcgtacattgcggagattactcgcggcgctgttctttccattcacaaaggttttcgtgaggccggtttagctcttgggctttcccgctgggaaacaattcgttatgttatcttgccgcttgccttgcgccgtatgttgccgccgctgggtaaccaatggatcatttctatcaaagatacttcgcttttcattgttattggagtggctgaattaacacgccaaggtcaagaaatcatcgcggggaatttccgtgcattagagatctggagtgctgtcgccgttttctacttgatcattacgctggtgctgtcctttattttgcgccgcttggagcgtcgcatgaagattctt

SEQ ID NO: 44 - DNA, 720 bp, Escherichia coli
  misc_feature 1..720: note = Glutamine Permease Q glnQ (Escherichia coliEPEC C342-62)
  source 1..720: mol_type = genomic DNA
atgattgaatttaagaatgtgtcgaagcatttcggccccacccaagtacttcacaacattgaccttaacatcgcccagggcgaggttgtagtaatcatcggtccatctggtagtggcaagtccaccttgctgcgttgtatcaataaacttgaggaaatcaccagcggagacttaattgtggacggtcttaaagtcaacgatccaaaagtggacgaacgcttgattcgtcaggaagcgggtatggttttccagcagttctacttgtttccgcaccttacggctcttgagaacgtcatgttcggaccgttacgcgtgcgcggggccaataaggaggaggcggagaagttggcacgcgagctgttagcaaaagttggcttggctgaacgtgcacatcattacccttctgagctgtcaggtgggcaacagcaacgtgtcgccatcgcacgcgcgcttgctgtaaaaccaaagatgatgctgttcgatgagccaacgtcggcgcttgacccggagttgcgccatgaggtccttaaggttatgcaagacttagctgaagagggaatgacgatggtaatcgtgacgcacgagattggattcgcagagaaggtagcatctcgtttgatcttcatcgacaaaggtcgcattgcagaagacggcgacccacaagttctgattaagaaccccccttcacagcgcctgcaagaatttctgcaacatgtctcc

SEQ ID NO: 45 - DNA, 1437 bp, Ustilago maydis
  misc_feature 1..1437: note = tryptophanamino transferase (transaminase) (Ustilago maydis 521)
  source 1..1437: mol_type = genomic DNA
atgagttccgccacaagtccggcactggattatgcattgctgttgtcttcttctgctcgtaaccgtatgccttctgcaatccgttccctgttcccggcagaattaattccaggcatggtctctcttttgtcaggtaaaccgaattcggagacctttccctttcagcgcatcagtttggaacttaaaccctccatccatctggagggacagaccgagacagtgagcatcgaaggtagcgatttagacatcgctcttcagtattcagcaacgagtgggttgccaaagttggtagactggatcattaaatttcaatctcgcgttcacgctcgtaagcaggtcgatgagggcaataagccgggtgaagtatggcgctgtagctttggcaacggatctcaagacctgctgaccaagacatttgaggctttagttgacgccggtgattcagtagtcctggaaagtccggcttacagtggaattttgccgtcgttggttgcgcataaagccaaccttttcgaggcagaaactgacgccgagggcgttgagcccacggctttagacacattgctgactaactggaagactgacagtgcaacacgtgactctcgttttcccaagtttttatatactaccccgactggtgcaaatccgtccgggacatcagcctctgataatcgcaagcgtgcgatccttgatattatccgcaagcacaatttacttctgctggaggatgatccttactattttttgtcattccaagggttggaaccgggggctgacgcggtcaaacgcactcgtgggaagagctattttcagttggaagctcaggacgactatggcgtcggccgtgttgttcgctttgattcatttagtaagatcttgtctgccggattacgcctgggtttcgttacaggacccaaagagattctggacgccatcgacctggacacttcctcccgcaatttgcagacaagtggcacttcccaggcaatcgcctatgctttgttgtctaagtggggaattgacggttttttacatcatgcggacaatgtcgcacgtttttaccaaaatcgcttagaacgctttgaagccagtgcccaggcaatcttaaccggaagccctagcatcgcctcgtgggttcgtccttcggcagggatgttcctgtggatcaagttaaagttgcctccgtcgcccgactcggcggagggtgatagttttgacctgatctctaataaagctaaggcagctggggtattggctttacccggtgtggccttcaaaccaccgagcagttcaagtacgggtggcaaacgtaagacatcggcatatgtccgcacgtcattctcccaggtgcctctggaccaagtggataccgcattcacacgcctgcgtcaggtggtagaggaggcctggcgtgaggctggacttcaaatccccgcg

SEQ ID NO: 46 - DNA, 1242 bp, Escherichia coli
  misc_feature 1..1242: note = Mtrtryptophan ArAAP transporter (Escherichia coliBL21(DE3))
  source 1..1242: mol_type = genomic DNA
atggctacccttactactactcaaacttccccatcgcttcttggaggagtcgttatcatcggtggaactatcatcggagcagggatgttttcactgccggttgtgatgtcgggagcatggttcttttggtcaatggcggctcttatcttcacgtggttctgtatgttgcatagtggcctgatgatcctggaagcaaatctgaactaccgtattgggtcctcttttgatacaattacaaaggaccttctggggaaaggatggaatgtagttaatggaattagtatcgcgttcgtcctttacatcttgacctacgcgtatatctctgcctcagggagcatcttgcatcacacttttgccgagatgtcattgaacgtgcccgcacgcgctgctggctttggttttgcactgcttgtggcattcgtagtctggttaagtacgaaggctgtgagccgtatgaccgctatcgtccttggggctaaagtaattaccttctttttaacattcggctcgctgttaggacacgtgcagcctgccactttgttcaatgtggctgaatcaaacgcctcgtatgccccctatttacttatgactttgccgttttgtctggcttccttcggttatcacggaaacgtgccatcactgatgaaatattatggtaaggatcctaaaacaattgtgaagtgcttggtatacgggaccttaatggcacttgccctttacacgatctggcttcttgcaacgatgggcaatattcctcgccctgaatttatcgggatcgcagaaaaaggggggaatattgacgtgctggtccaggctttatcgggtgtcttgaatagccgctctttggatcttttgttagttgtcttttccaattttgccgtggcatcgagtttcttaggtgtgacgctgggtctttttgattacctggccgatctgttcggattcgacgacagcgcggtgggccgtcttaaaactgctttattaacatttgcgccccctgtagtgggaggtcttctgtttcctaacggattcttatacgccatcggctacgccggattggcggccacgatttgggcagctatcgtcccggctttattggcacgtgcctcacgcaaacgcttcgggagtcctaaattccgtgtttggggcgggaagcctatgattgcccttattttagtgtttggagtcggtaatgcacttgtgcacatcttgtcatcgttcaatctgcttcccgtttatcaa

SEQ ID NO: 47 - DNA, 1245 bp, Escherichia coli
  misc_feature 1..1245: note = tryptophan permeaseTnaB (Escherichia colistr. K-12 substr. MC4100)
  source 1..1245: mol_type = genomic DNA
atgaccgaccaagctgaaaagaagcattcggcattctggggagtaatggtcattgccggtaccgtgatcggcggtgggatgtttgctttacctgtggacttagcaggcgcgtggtttttttggggggcgttcattctgattattgcttggttttccatgctgcatagtggcttgctgcttcttgaagcgaatcttaactatccggtggggtcaagtttcaataccattacaaaggacctgattggtaacacatggaatatcatttcggggatcacggtagcatttgtattgtatattcttacatatgcttatatcagtgcgaatggcgcaatcatttccgagacgatctccatgaacctggggtatcacgcgaatccccgtattgtcggcatctgcacagcgatttttgttgcgagcgtattatggctgagttcgttggcagcttcgcgtattacttcccttttccttggtttgaaaatcatcagcttcgtaattgtgtttgggagtttttttttccaggtcgactactccattcttcgcgatgcaacaagtagcacagcaggcaccagttacttcccatatatctttatggccttaccggtttgtttagcgtcttttggttttcatggtaatatcccctcattaattatttgctacggcaagcgcaaggacaaattaattaagtctgttgttttcggctccttgttggcgcttgtaatctatttattttggctttattgtacgatggggaacatccctcgcgaatcctttaaggctattatttcttcaggaggcaacgtagacagtttggtaaaaagttttttgggtacgaagcagcatggtatcatcgagttttgtttacttgttttcagtaatcttgccgttgcttcctcattctttggcgtgactctggggctttttgattatctggcagatttattcaagatcgacaactcgcatggcgggcgcttcaaaacggttctgcttacatttcttcctccagctttactttacctgatctttccgaatggttttatctatggtattgggggggcaggcctgtgcgccactatctgggcagttatcattcctgctgtattggctatcaaggcacgcaaaaagtttcccaaccagatgttcaccgtgtggggcggcaatttgattccggcaatcgtgatcttatttggtatcacggttattctttgctggttcggcaatgtgtttaacgtcctgcctaagtttgga

SEQ ID NO: 48 - DNA, 1368 bp, Escherichia coli
  misc_feature 1..1368: note = aroP (Escherichia coliO104:H4 str. C227-11)
  source 1..1368: mol_type = genomic DNA
atggaagggcagcagcatggcgaacagcttaagcgtggcctgaagaatcgtcatatccagcttatcgcattaggcggagctattgggaccggcttgttcttaggctctgcttcagtcattcagtctgcggggccaggcattattttaggctacgcgattgcgggcttcatcgcctttttaattatgcgccagcttggcgagatggtggtggaggaacccgtggcaggcagtttctctcactttgcatacaagtattggggaagttttgcaggctttgcgagcggttggaactactgggttctgtacgttctggtggccatggcggaactgacagcagtcggtaaatatattcaattctggtaccctgaaattcccacttgggtctctgccgctgtcttctttgtcgtcattaatgcaatcaacttgaccaacgtcaaagtattcggcgagatggagttttggttcgctattatcaaagtcattgctgttgtggccatgatcattttcggaggctggctgcttttcagcggcaacggaggtccccaggcaactgtatcgaatctttgggaccagggtggtttcttgccacatgggttcacggggttagttatgatgatggccattattatgttctcgtttggagggcttgaattggtgggcatcactgctgctgaagctgataacccggagcaaagcattcctaaggccacaaatcaagtgatctatcgcatccttatcttttacattggatcgttggcagtattgctgagtttgatgccctggacccgtgtcaccgctgatacaagcccttttgttttgatttttcatgaattaggggatacttttgtggcaaatgcgttaaacatcgtcgtattaactgctgccttgtcagtatataactcctgcgtatactgtaatagccgtatgctgttcggcttggctcagcaggggaacgctccgaaagcactggccagtgtcgacaagcgtggagtacctgtgaatacgattttagtttctgctctggtcactgcactttgtgtattgatcaactacctggcgcctgagtcggcgtttggcctgctgatggcgctggtggttagcgcattggtcatcaattgggcgatgatctccttggcacacatgaaattccgccgtgctaaacaagaacagggtgtggttacacacttcccagcattattataccctctgggcaactggatttgcttactttttatggcagcggttctggtcatcatgctgatgacgcctggtatggctatttctgtttatctgattccggtttggttaatcgtattagggattggctatttattcaaggaaaaaactgcaaaggctgtcaaagcgcat

SEQ ID NO: 49 - DNA, 879 bp, Escherichia coli
  misc_feature 1..879: note = Aromatic amino acid exporterYddG (Escherichia coliTW10598)
  source 1..879: mol_type = genomic DNA
atgacccgccagaaggcgactctgatcggtttgattgctatcgtattatggtccacaatggttggtttaattcgtggggtttctgaggggcttggcccggtgggcggagcagcagctatctactccctgagcggtctgttattgatctttacagttgggtttccgcgtatccgtcaaatccccaagggatacttattggcggggagtttactttttgtgagctatgaaatttgccttgccttgtctctgggctacgcagcgacacgccatcaagcaattgaggtagggatggttaattacctttggccgtcattgacgattcttttcgcaatcttatttaacggtcagaagactaattggttgattgtaccgggtttattattagcgttggtgggagtatgctgggtgttgggaggtgacaatggtctgcattatgacgagattattaataatatcacaacatcgcccttatcctactttctggctttcattggtgcctttatctgggccgcctattgcaccgtgacgaataagtacgctcgtggcttcaacggaattacagtatttgtcttgcttactggtgcatctttgtgggtatattatttcttgacccctcaaccagagatgatcttctccaccccggttatgatcaaattaatttcagcagctttcactttgggattcgcatacgcagcttggaatgtcggcattcttcatgggaatgtgacgattatggcagtcggttcctacttcacgcccgtacttagttccgctttagcagcggtactgctgtcggcgcctttgagttttagtttctggcagggtgccctgatggtgtgtgggggctcccttttgtgctggcttgctacccgccgtggt

SEQ ID NO: 50 - DNA, 1152 bp, Escherichia coli
  misc_feature 1..1152: note = S-adenosylmethioninesynthase Escherichia coli (strain K12)
  source 1..1152: mol_type = genomic DNA
atggcaaagcaccttttcacgtcggaatctgtatctgaagggcatcccgacaaaattgcagatcaaatctccgacgcggtacttgatgctattctggaacaagatcccaaagcccgcgtcgcttgcgaaacttatgtcaagacaggcatggtgttagtcggcggcgagatcactacctctgcgtgggtggatatcgaggaaatcacgcgcaatacggtgcgtgagattggctatgtacactcggacatggggttcgacgccaacagttgtgcggttttaagtgccattgggaaacagtcacctgatattaatcagggggtggatcgtgcggaccctcttgaacaaggtgctggtgaccaaggtctgatgttcggttatgctacgaacgaaaccgatgtgttgatgcccgccccgatcacatacgcccaccgtctggtccaacgccaggcggaggtccgtaaaaacggcacgcttccttggcttcgtccagatgctaagtcgcaggtcactttccaatacgacgacgggaagattgtcggaatcgacgccgtggtcttgtcaactcagcattcagaggagatcgatcaaaagagccttcaggaagccgtcatggaagagatcatcaagccgattctgcctgcagaatggttaacttccgcgaccaagttctttattaaccccaccgggcgttttgtcattggcggtcctatgggcgactgtgggttgaccggccgtaaaattattgtcgacacttatggcggaatggctcgtcatggcggtggggcattcagtggcaaggacccgtcaaaggtagatcgttcagccgcctatgccgcccgttacgtagccaagaacattgttgctgcaggacttgctgaccgctgtgaaatccaagtgagctacgcgatcggcgtagcagaacccacctccattatggtggaaacttttggcaccgaaaaagtccccagtgagcaactgaccttattggttcgtgagttttttgatttgcgcccttacggacttatccaaatgttagaccttttgcacccaatctacaaagaaactgcagcatacggtcactttggacgcgagcattttccctgggagaagacagacaaagcacagctgttacgtgacgcggccggattgaaa

SEQ ID NO: 51 - DNA, 1275 bp, Anabaena cylindrica
  misc_feature 1..1275: note = adenosylhomocysteinase (Anabaena cylindrica PCC 7122)
  source 1..1275: mol_type = genomic DNA
atgacggctacgacgccacgcctgaaacatgaagtgaaggaccttgcgcttgcgcctttaggtcgtcagcgtattgagtgggcggggcgcgaaatgcctgttttaaagcaaatccgcgaccgctttgaaaaagaaaagcccttcgcgggcctgcgtatctcggcttgtgcgcatgttacaacagagacggctcatttagcaattgccctgaaggccgggggagctgatgccgtattgatcgcaagcaacccactgtctacgcaggatgacgtagcagcctcgcttgtcgctgatcatgagatctctgtgtttgcacaaaagggcgaagacgccgcgacgtactcgcgtcacgtccaaattgcgttggaccaccgccccaatatcatcgttgatgacggttccgacgtagtagctgaattagtacagcaccgtcagaatcagatcgcggatcttattggatccactgaagaaactacaactgggattgttcgccttcgcgctatgttcaacgagggggttttgacgtttcccgcgatgaatgtcaacgacgcagacacaaaacatttttttgacaaccgctacggtacaggacaatctaccttggacgggatcattcgtgcaaccaacatcttgcttgccggcaaaactatcgtagttgtaggctatggctggtgcggaaaggggaccgcattacgcgcccgcgggatgggagctaatgtcattgttaccgagatcgatcacattaaggcaattgaggcggtgatggatgggtttcgcgttctgcccatggctgaagccgcaccgcatggtgatatctttatcactgtaacgggtaataaacacgtagttcgtggtgaacactttgatgtcatgaaagacggcgccattgtttgcaactcaggtcacttcgatttggagttggatttaaaatatttagcagcaaatgccaaggaaatcaaagatgtgcgcccattcacacaagaatataaattaaccaacggcaaaagcgtagtggtattaggagaggggcgtttgattaatcttgcagcggcagaaggtcatccgtcggcagttatggacatgtctttcgccaatcaagccttagcagtcgagtatttagtgaaaaataaaggctccttggcggctggattacattcgatcccccgcgaggttgatgaggaaatcgctcgtttaaaattgcaagcgatggggatttttatcgattccctgacagcagatcaaatcgattatattaattcttggcagtcagggacg

SEQ ID NO: 52 - DNA, 1374 bp, Klebsiella quasipneumoniae
  misc_feature 1..1374: note = Cystathionine-beta-synthase (Klebsiella quasipneumoniae subsp. Quasipneumoniae)
  source 1..1374: mol_type = genomic DNA
atggtaatgtcgttattccacagtgttagcgatttaatcggtcacacacctttattacaattgcataagcttgatacaggaccctgtagtttgttcttgaaacttgagaatcaaaacccaggagggtcaattaaagatcgtgtagcgcttagcatgattaacgaagcggaacgtcagggaaaacttgcgccaggaggaactatcatcgaggctacggcgggaaatactgggttggggcttgctttgatcgcagcccagaaaaactaccgtcttatccttgtagttcccgacaagatgtcacgtgaaaaaattttccacttgcgtgccttaggcgcaaccgtgcttttgacccgttcagacgtgaacaaggggcacccggcatattatcaggactatgctcgccgcttggcagatgagactccaggggcgttctacattgaccaattcaataatgatgccaatcctttagcacatgcaacaagcacggcccctgagctgttccaacaattagaaggggacatcgatgccattgtggttggtgttgggtcgggtggaacgttgggcggcttgcaggcctggttcgcagaacactctcccaaaacagagttcatcttggctgatccagctgggtcgattcttgccgaccaggtagacacaggccgctacggggaaacgggaagctggcttgtagagggtattggcgaggattttatcccaccacttgctcgcctggaaggagttcataccgcatatcgtgtatctgatcgcgaagcctttcttacagcccgtcaactgcttcaggtagagggtgtattagcgggctcgtcaacgggaacattgttatctgcggccttgcgctattgccgtgcccagtctcgcccaaagcgtgtggttaccttcgcatgtgactctggaaataagtacttgagtaagatgttcaatgacgactggatgcgccaacagggacttattgcgcgcccggaacagggagatctgagtgatttcatcgccttacgtcacgacgagggggccacggtcaccgccgcgcccgacgacacactggcggctgtatttactcgcatgcgcttgtacgatatctcccagcttccggtcttggaagacggtcgtgtcgttggcattgtggacgaatgggatttaattcgccatgtacgtggcgaccgtcaacgcttttccctgccagtcagcgaggctatgtcccgtcacgtagaaacgttagacaaacgcgcccccgaatccgaattgcaagctatcttagaccgtggactggtagcagtcattgcagacaatgcgcgctttctgggactggttacacgttcagatgtcttaacggcatggcgcaatcgtgtggcgcaa

SEQ ID NO: 53 - DNA, 1146 bp, Klebsiella pneumoniae
  misc_feature 1..1146: note = cystathionine-gamma-lyase (Klebsiella pneumoniae subsp. pneumoniae HS11286)
  source 1..1146: mol_type = genomic DNA
atgtcgtctattcacaccctgtctgttcatagtggcaccttcacggactcacatggcgcggtgatgcccccaatctatgccacctccacgttcgcgcaacctgcgcccggacagcacaccggatatgaatactcgcgcagtggaaatcctactcgtcatgccttagagactgcgatcgcagacctggagaatggaacgcgcgggtacgcatttgcctcgggcttggcagcgatctcgactgtccttgaattgttggataaggacagccatttagttgcagtggatgatgtctatggtgggacctaccgtttacttgaaaacgttcgtcgtcgttctgctgggctgcaagtgtcgtgggtcaagccagacgatttagcggggattgaggcggctatccgtcctgacacccgtatgatctgggtcgaaacacctactaatcctttgctgaaattagccgatttgagcgccatcgcagctatcgcacgccgtcacaatcttatttcagttgcggataacacgttcgcttcaccagccatccaccgtcctcttgaacacggtttcgacattgtggtgcattctgcgacaaaatacttaaatggacattccgatgtggttgcggggttagctgtcgtcggagataactccggcttagccgagaaattaggttatttacaaaatgcagttggcggggtattagaccccttttcctcgttccttacattgcgcggcatccgcactctggcactgcgtatggaacgtcatagcgcgaatgcactgcagttagccgaatggttggaacaacagcccgaagtagagcgtgtatggtttccttggctggcctcccatcctcatcatcaattggcacgtcagcagatggcattacctggcgggatgattagcgtagtagtcaaaggagatgagggatatgctgagcgcatcatcagtaaactgcgttggttcactcttgccgagtctttaggcggcgtcgagtcgttagtttcccagccgttctcaatgacacatgcttcgatcccacttgaaaagcgtcttgcgaacggcattacgccccagcttattcgccttagtgtggggatcgaagacccacatgatcttatcgcggattggcaacaagccctgcgtgccgaa

SEQ ID NO: 54 - DNA, 483 bp, Bacillus subtilis
  misc_feature 1..483: note = cysteinedioxygenase (Bacillus subtilis subsp. subtilis str. BAB-1)
  source 1..483: mol_type = genomic DNA
atggagttatacgagtgcatccaggacatcttctcggggttgaaaaacccttccgtgaaagatctggcaacatccctgaaacaaatcccgaatgcagctaaattatctcagccttacattaaagagcctgaccagtatgcatacggtcgcaatgccatctaccgtaacaacgagttggagattattgttatcaacattcctcccaacaaagagacaaccgtacacgatcacggacaatccattggatgcgcaatggttctggaaggtaaattacttaatagcatttatcgttctgctggtgagcacgccgagctgtccaactcttactttgttcacgagggggaatgccttatctcgactaaaggcttgattcacaaaatgagcaaccccacaagcgagcgcatggtatcgttgcatgtttattcgccaccgcttgaggacatgacagtatttgaggaacagaaagaggtgttaaagaactct

SEQ ID NO: 55 - DNA, 891 bp, Caenorhabditis elegans
  misc_feature 1..891: note = Glutamate Oxaloacetate Transaminase (Caenorhabditis elegans)
  source 1..891: mol_type = genomic DNA
atgagcgttagtaaaaaactgttctctacggctgtgcgtggtaagagctggtggtcacacgtcgagatgggccctcctgatgcgattttgggggtgactgaagctttcaaagctgattctaaccccaagaagatcaatttgggcgtgggagcgtaccgtgatgaccaaggaaaaccgttcgtacttcctagcgtcaaggaagccgaacgtcaagttattgcagcaaatcttgacaaggagtacgccgggatcgttggcctgcctgaattcacgaaacttagtgctcagttagcattaggggaaaacagtgacgtaatcaaaaacaagcgtatttttacgacgcaaagtatttctgggactggtgcgctgcgtattggaagtgagttcctgagtaaatatgcaaagactaaggttatctatcaacccacgcctacatggggaaaccacgtgcctatcttcaagttcgcgggcgtggatgtgaaacagtatcgttattatgacaagtctacatgtggatttgatgagacgggggcattggctgatattgcgcaaatccccgaaggtagcactattttgctgcacgcgtgcgcacataacccaacgggggtcgaccctagtcgtgaccaatggaaaaagatttcagatattgttaagaaacgcaatttgttcgtgttttttgacatggtgaatgagtcagtcctgagtccgttactgcctcgcacgcttatgcgcctgcttgtgttgttactgaaatcccgcagtcttttcgcccactcaacacccacccatcagtcgatggaattagctcttttgccggcctcgtcgcgtatccaactttctacctccaatgggtcagaaatgtccagctcttggcttatcgtcagcagccct

SEQ ID NO: 56 - DNA, 1179 bp, Bacillus halodurans
  misc_feature 1..1179: note = methionine gamma lyase (Bacillus halodurans C-125)
  source 1..1179: mol_type = genomic DNA
atgaaacgcgaccaacattttgaaacacgcgcgatccatactggttacaagccgaacgagcattttgatagcttgactccccctatttaccaaaccagcacgttcacatttgcatcaatggagcaaggtggcaaccgtttcgcaggcgaggaagcaggatatgtttattcacgcctggggaaccccaccgtgcaaattttggaacaacgcattgctgagttggagggtggggaggcagctcttgcctttggatctggcatggctgctgtcagtgcgattttggtggggcttacgaaggccaacgaccacatcttagtgagcaatggagtgtatggttgtacgtttgggttgttaacgatgttaaaggaaaaatacaacatcgacgccactttcagtccgatggacagcgtagaggaaatcctggcaaacatccaggataataccacgtgcatttatgtggaaacacctatcaaccccaccatgcagttaatcgatttggaactggttgtgcgcgtagcgaaggaaaagggtattaaggtaatcgttgataacacgtttgccacaccatacttacaacaaccgattgctctgggatgtgacttcgttgtccattcggccacgaaatacatcgggggtcatggggacgtggtcgccggagtgctgattggagacaaggaaacaattcagttgatccgtaagaccacccagaaggatatggggggcgtaatttctccatttgatgcgtggctgctgttgcgcggattgaaaacacttgcagtacgtatggatcgccattgcgagaatgctgaaaaattggccgagaaactgaaagagcatccaaaagtaagtacggttctgtacccgggagactttgagcatcccgatcactccatcgtcgccaaacagatgaaaaagggaggcggtttattaagctttgagatcaaggggactgaggcggacatcgccaaagttgtaaatcagttaaaactgattcgtattgctgttagtttgggtgacgcagagaccttgattcagcatcctgcaaccatgacccatgcagtagtacccgaaaagcgccgcactcaaatgggtattagtaaaaagttgttacgcatgtcggccgggttagaggcctggcaagatgtctgggctgacttagagcaggcgttaaatcaactg

SEQ ID NO: 57 - DNA, 1269 bp, Methylobacterium aquaticum
  misc_feature 1..1269: note = Methionine aminotransferase (Methylobacterium aquaticum)
  source 1..1269: mol_type = genomic DNA
atgaccgcgattccggccttggcagacctgcaggctcgttatgccgacttacaagggcgtggtctgaagttagatatgacgcgcggtaaaccggcgccagagcagttggatttatcggacgatcttttcactttaccaggtaaccgcgatcaccgcacagagagcggagaagacgcgcgtaattacggcggagtacagggcctggctgaggtccgtgccttattcgcccctgtgcttggtgcgtcacccgatcgcattgccgtaggtaataactcatcgttggcattgatgcatgactgcattgcctatgcattgcttaagggtgtacccggcggcgctcgtccttgggcaaaggaagaggagattcgttttttatgcccagtcccagggtacgaccgtcacttcgctctgtgcgagacctacgggattggaatgattccagtccctatgaccgctgacgggcctgatatggaaatggttgaacgtgaggtacgcgatccacgcgtcaaaggtatgtgggcggtgccgcagtatagtaacccaggcggtgagacatactccgacgcgactgttgagcgcctggctcgtatggaaaccggtgcccctgacttccgtcttttttgggacaacgcgtatgcacttcaccatttgaccgaacgtcgcccaacccttcgtaatgtgttagatgcctgtgcggaagccgggtcaccggatcgtgctattgtgtttgctagtacgtcgaaagttacactggcgggggcaggccttgcgatgcttgcgtccagcgagggcaatattcgctggtatttagctaacgccggcaaacgctcaattggtccagataagcttaaccagttgcgccatgttcgctttctgcgtgaccagggcggacttgatgcattaatggacggccaccgccgtcttttagctcctaagttccgcgctgtaacggaaacccttgctcgtcatctgggcgggactggagtagcgcgctggagcgagccggaaggggggtactttatcctgctggaagtccctgagggctgtgcgacacgcgtagttaagcttgctgctgcttgcggactggctctgacgcccgcaggggcgacgcacccatacgggcgtgaccctcaagataagctgttacgtcttgccccgtcatacccgaaaccagcggaggtcgaggcagccgctgaggtagtcgctgtgtgcgttttacttgcggcagctgaaagccgcgaagctggcggttcggggcaggttgctgca

SEQ ID NO: 58 - DNA, 1902 bp, Saccharomyces cerevisiae
  misc_feature 1..1902: note = Aro10p decarboxylase (Saccharomyces cerevisiae YJM1615)
  source 1..1902: mol_type = genomic DNA
atggcacccgtcactattgagaaattcgtgaatcaagaagagcgtcatttagtgagcaatcgttccgccacgatcccttttggagaatatattttcaagcgccttctttccattgacaccaaaagcgtcttcggggttcccggcgacttcaatttatctttattggaatatttatactcgccctccgtggaatctgcgggtcttcgttgggttggcacctgtaacgagttaaatgcagcctacgctgcagatggatattcccgctactctaataaaattggatgcttaatcaccacatacggcgtaggagaactgagtgcgcttaatggaatcgcggggtcattcgctgaaaatgtaaaggttctgcatatcgtaggggtcgccaagtccattgattcccgttcgtctaacttctcggatcgtaacttacatcacttggtcccgcagttacatgattcgaactttaaaggacccaaccataaggtctatcacgacatggttaaagatcgtgtcgcatgttccgtcgcctacctggaggatattgagacggcctgtgaccaagttgataacgtgatccgtgacatttataagtattcaaaacctggttacattttcgtcccagccgactttgccgacatgtccgtaacctgcgacaacttggtcaatgtaccgcgtatcagccaacaagattgtattgtctaccccagcgagaaccaactgtcagacatcattaataaaatcactagctggatctactcgtctaagactccagcaatccttggagacgtcttaactgatcgttatggggtatcaaactttctgaacaaactgatctgcaaaaccggtatctggaacttctccaccgtgatgggaaaatcagtcattgacgagagtaacccaacttatatgggtcaatacaacggcaaagaaggtcttaaacaggtctatgaacatttcgagctgtgtgatttggttttacacttcggagtagatattaacgagatcaataatggtcactacacgttcacttacaagccaaatgcgaaaattattcaattccaccctaattatattcgtttagtagacactcgtcaggggaatgaacaaatgttcaaaggcatcaattttgcgccaatcttgaaagagttgtataagcgtatcgacgtctctaaattatcgttgcaatacgattccaatgtaacacaatacaccaatgagactatgcgtctggaggacccaacgaatggtcaatcgagcatcattacccaagtacacctgcaaaagaccatgccgaaatttttgaatcccggcgacgtcgtcgtgtgtgagactggtagtttccaattcagtgtacgcgacttcgcattccccagtcagttgaaatatatcagccagggtttctttttatccattggtatggccttgcctgccgcgttgggggttgggatcgcaatgcaggatcattccaacgcgcatattaacggagggaacgtcaaagaagactacaagccccgcttaattttgtttgaaggtgacggcgccgcgcagatgaccatccaggagcttagcacgatccttaaatgcaatatccctttggaggtcattatctggaataacaatggatacactatcgagcgtgccatcatgggtccaacacgttcatataacgatgtgatgtcgtggaaatggacaaagttgttcgaagcctttggggatttcgatggtaagtatacgaattcgactttaattcagtgtcctagcaaattagcgttaaaacttgaagaattgaagaattctaataagcgttcggggatcgaactgttagaagtgaagctgggtgagcttgacttcccagagcaattgaagtgtatggtagaggccgcagctcttaaacgtaataag

SEQ ID NO: 59 - DNA, 666 bp, Bacillus subtilis
  misc_feature 1..666: note = Methionine import system permease proteinMetP (Bacillus subtilis)
  source 1..666: mol_type = genomic DNA
atgtttgagaagtattttccaaatgttgacttgaccgagttatggaatgccacatatgaaactctgtatatgacattgatttccttactgtttgccttcgtaatcggcgtcatcctgggattgctgttattcttaacatctaaggggtctctttggcaaaataaagcagtaaattccgttatcgcagccgttgtcaacatctttcgttcaattcccttccttattttaatcatcctgcttcttggtttcactaaattcttagtgggaacaattttgggaccaaatgcggctcttcccgcgttagtcatcggtagtgctcccttttatgctcgtctggtcgaaatcgcacttcgtgaagtggacaaaggagtgattgaggcggcgaaatcgatgggggctaagacgagcactattatttttaaggttcttatccccgagtccatgcccgcgctgatttccggaattacagtgactgcgattgcattgatcgggtcaaccgccatcgcaggagctattggttctggtggattgggaaacttagcatacgttgaaggctatcaatcgaataatgcggatgtgaccttcgtggccacagttttcatcctgattattgttttcatcattcagatcattggtgaccttattaccaacatcatcgataaacgc

SEQ ID NO: 60 - DNA, 1029 bp, Escherichia coli
  misc_feature 1..1029: note = DL-methionine transporter subunit MetN (Escherichia coli K-12])
  source 1..1029: mol_type = genomic DNA
atgattaaactgagcaacattactaaggtgttccaccaaggtacacgtacgatccaggctcttaataatgtgtcactgcacgttcctgctggtcagatttatggggttatcggtgccagtggggctgggaagagcactctgatccgctgcgtcaatctgttagagcgccctacagagggctcggtactggtggacggtcaagagttgactactctgtcggagtccgagttgacaaaagcacgccgccagattggcatgattttccaacatttcaatttgttatcgagccgtacagttttcgggaacgtggccttaccactggagttggacaatactcccaaagacgaagtcaaacgtcgtgtgaccgaattattgtccttggtgggtcttggtgacaaacacgacagttatcccagtaatttgagtggcgggcaaaaacagcgtgttgccatcgcacgcgcattagcttcgaatcccaaggtgctgttatgtgatgaagcgaccagcgcccttgacccagccacaactcgtagcatcctggagcttttgaaagatatcaatcgtcgcctgggtttgaccatcttattgattacgcacgagatggacgttgtaaagcgtatctgtgactgtgtagcggtgatctccaacggtgaattaatcgaacaggacaccgtatcggaggtcttctcacatcctaagacaccccttgcacaaaaattcatccaaagcacgctgcatttagatattcctgaagattatcaggaacgcctgcaggctgaaccgtttactgattgcgttccaatgcttcgcttagagttcacagggcaatcggttgacgctcccttattgagtgaaaccgcccgccgtttcaatgttaataacaacatcatttccgcgcaaatggactacgcggggggtgttaaatttggaatcatgttaaccgaaatgcacggcacacagcaggatacacaggcggcgatcgcatggctgcaggaacatcatgttaaagtagaagtccttgggtatgtg

SEQ ID NO: 61 - DNA, 654 bp, Escherichia coli
  misc_feature 1..654: note = metI (Escherichia coli)
  source 1..654: mol_type = genomic DNA
atgtctgagccgatgatgtggctgctggttcgtggcgtatgggaaacgctggcaatgaccttcgtatccggtttttttggctttgtgattggtctgccggttggcgttctgctttatgtcacgcgtccggggcaaattattgctaacgcgaagctgtatcgtaccgtttctgcgattgtgaacattttccgttccatcccgttcattatcttgcttgtatggatgattccgtttacccgcgttattgtcggtacatcgattggtttgcaggcagcgattgttccgttaaccgttggtgcagcaccgtttattgcccgtatggtcgagaacgctctgctggagatcccaaccgggttaattgaagcttcccgcgcaatgggtgccacgccgatgcagatcgtccgtaaggtgctgttaccggaagcgctgccgggtctggtgaatgcggcaactatcaccctgattaccctggtcggttattccgcgatgggtggtgcagtcggtgccggtggtttaggtcagattggctatcagtatggctacatcggctataacgcgacggtgatgaatacggtactggtattgctggtcattctggtttatttaattcagttcgcaggcgaccgcatcgtccgggctgtcactcgcaagtaa

SEQ ID NO: 62 - DNA, 816 bp, Escherichia coli
  misc_feature 1..816: note = metQ (Escherichia coli)
  source 1..816: mol_type = genomic DNA
atggcgttcaaattcaaaacctttgcggcagtgggagccctgatcggatcactggcactggtaggctgcggtcaggatgaaaaagatccaaaccacattaaagtcggcgtgattgttggtgccgaacagcaggttgcagaagtcgcgcagaaagttgcgaaagacaaatatggcctggacgttgagctggtaaccttcaacgactatgttctgccaaacgaagcattgagcaaaggcgatatcgacgccaacgccttccagcataaaccgtaccttgatcagcaactgaaagatcgtggctacaaactggtcgcagtaggcaacacttttgtttatccgattgctggttactccaagaaaatcaaatcactggatgaactgcaggatggttcgcaggttgccgtgccaaacgacccaactaaccttggtcgttcactgctgctgctgcaaaaagtgggcttgatcaaactgaaagatggcgttggcctgctgccgaccgttcttgatgttgttgagaaccccaaaaatctgaaaattgttgaactggaagcaccgcaactgccgcgttctctggacgacgcgcaaatcgctctggcagttatcaataccacctatgccagccagattggcctgactccggcgaaagacggtatctttgttgaagataaagagtccccgtacgtaaacctgatcgtgacgcgtgaagataacaaagacgccgagaacgtgaagaaattcgtccaggcttatcagtctgacgaagtttacgaagcagcaaacaaagtgtttaacggcggagctgttaaaggctggtaa

SEQ ID NO: 63 - DNA, 2286 bp, Bacillus atrophaeus
  misc_feature 1..2286: note = MetE (Bacillus atrophaeus UCMB-5137)
  source 1..2286: mol_type = genomic DNA
atgacgactatcaaaacatcaaatctgggcttccctcgcattggacttaatcgcgaatggaaaaaatcactggaagcgttttggaaaggtaacagcgacaaagatacatttcttaagcagatggatgagttatttcttactgccgtaaaaacccagattgatcaaaaaatcgacatcgtgcccgtgagcgacttcactcactacgaccacgttcttgacacagctatctcttttaattggattccagaacgctttaaacacattacggatgcgactgatacatatttcgcgctggcacgtggcattaaggatgctgttagttcggaaatgactaagtggtttaataccaattaccactatatcgttccggaatacaataaagacatcgaattccgtttaacccgcaacaagcagttagaggactaccgccgcgtcaaacaagcgtttggcgtcgaaactaaacccgtcattgtcggtccttacacattcgtgacgcttgccaagggctacgaacaaagtgaggccaaagaaatccaaaagcgtttagtcccattgtatgtgcaattattgaaagaattggaacaagagggcgtgcagtgggtacaaatcgatgagccagcacttgtgacagcctcatccgaggatgttagcgcggccaaggagttataccaggccattacgaatgagttatccggcttgaatgtccttttgcagacttacttcgattctgttgatgcttatgaggagttaatcagctacccggtacagggtatcggcttggattttgtacacgataaagggcgcaacttggagcaattaaaagcgcatggatttccgaaggataaggtattagcagctggtgttattgatggtcgtaacatttggaagacggatttagatgagcgcttggacgccatccttgcgctgttatcttcgacggacattgacgaattatggattcaaccaagcaattcgcttcttcatgtaccagtagcaaagcacccagacgagcacctggagaaggatctgttgaatggcttgagttacgcaaaagaaaagctggcagaactgtccgctttaaaagagggtttgttatcgggtaaagcggcaatctcggccgacattcagcaggccaaagcggatttacaggccctgaagcaattcgccaccggggctaacagtgagcagaaagaggaattaaatcagttgaccgagaaagactttaagcgcccgatccccttcgaagagcgcctgaaaatccagaatgaatccttggggcttcccctgcttcctactacgactattggttcttttcctcaaagcgccgaggtgcgttcggcgcgccaaaagtggcgcaaaagtgagtggagcgacgagcaatatcaagaatttatcaacgcggaaacgaagcgctggatcgacattcaggaagagcttgatcttgacgttttagtacatggagagttcgagcgcaccgacatggtcgaatatttcggtgagaaactggctggattcgcgtttactaaatacgcatgggtccagagctacggatcccgctgtgtacgccctcccgtcatctatggggacgtggagtttattgaacctatgactgtcaaggacacagtgtacgctcaatctttaacgagtaagcaggttaaagggatgttgactggcccggtcacaatcttgaattggagcttcccgcgtaacgacattagccgtaaggagatcgccttccaaatcgggttagctcttcgcaaagaggtcaaggcgttggaagatgctggtattcaaatcatccaagttgacgaaccggccctgcgtgaagggctgcctctgaaagaaaacgattgggaagagtatttaacgtgggccgcggaggcgttccgcttaactacttcggctgtgaaaaacgacactcagattcatacacacatgtgttattccaattttgaggacattgtcgacacaattaatgacttggatgcggacgtcattacaatcgaacactcccgcagtcacggtgggttcttggactacttgcgcgatcatccgtatcttaaaggtttaggtcttggcgtgtacgatattcacagccctcgtgtacccccgacagaggaaatttataagatcattgacgaagccctgaccgtatgtcctactgaccgcttctgggtaaacccagactgcgggctgaagacccgtcaccaggaggaaacgattgccgcgttgaagaacatggtcgaggctgctaaacaggctcgtgccaaacagagtcaacttgtc

SEQ ID NO: 64 - DNA, 753 bp, Corynebacterium glutamicum
  misc_feature 1..753: note = BrnF (Corynebacterium glutamicum)
  source 1..753: mol_type = genomic DNA
atgcagaaaacacaggagattcacagctcgttagaggttagccccagtaaagctgctctggagcccgacgataaggggtatcgtcgttacgaaatcgcacaaggcctgaagacctctcttgctgcaggcctgggaatgtatcctatcggaattgcattcggcttactggtgattcaatatggttatgaatggtgggccgctccactgttctccggcctgatttttgcggggtctacggagatgcttgtaattgcacttgtggtcggcgctgctccgctgggtgccattgcccttacgaccttacttgttaatttccgtcatgttttctatgccttttcctttcccttgcacgttgttaaaaaccctattgcgcgcttctattctgtattcgctcttattgatgaagcatacgctgttacagccgctcgtcccgccggttggagtgcatggcgtctgatttcaatgcagattgcgttccactcctactgggtatttggaggcttgaccggtgtagcaatcgcagagttaattcctttcgagatcaaaggcctggagttcgcactttgttcgttatttgtaactcttactttagacagttgtcgcactaagaaacaaattccgagtttgttattggctggactgagctttactatcgcgttagtagtgatccccggccaagctctgttcgctgcgttacttatctttctggggcttctgacaatccgttattttttcttagggaaggcagccaaa

SEQ ID NO: 65 - DNA, 324 bp, Corynebacterium glutamicum
  misc_feature 1..324: note = BrnE (Corynebacterium glutamicum)
  source 1..324: mol_type = genomic DNA
atgacgactgatttctcctgcatcctgttggtggtcgcggtatgtgcagtcattacatttgcgcttcgtgccgtaccttttctgatcttgaaacccttgcgtgaatcgcaatttgtgggaaaaatggccatgtggatgcctgcgggcattctggcaatcctgacggcttctaccttccgttcaaacgccatcgatttaaagacgttgacgttcggtctgattgccgtggcaatcacagtcgtagcccacttattaggaggccgtcgcaccttattatctgttggcgctggaacaattgtgtttgtaggtcttgttaatttgttt

SEQ ID NO: 66 - DNA, 1023 bp, Salmonella enterica
  misc_feature 1..1023: note = threonine 3-dehydrogenase (Salmonella enterica subsp. enterica serovar Typhistr. CT18)
  source 1..1023: mol_type = genomic DNA
atgaaggccctgagcaaattgaaagccgaggaggggatctggatgaccgatgttcctgaaccagaagtggggcacaacgaccttttaatcaaaattcgcaagactgcaatctgcgggacagacgtacatatctataactgggacgagtggagtcaaaaaactattcccgtccctatggtggtcgggcacgagtatgtcggagaggttgtaggaatcggacaagaagtcaaaggatttaaaatcggggatcgtgtgagtggggagggtcacattacctgtgggcattgccgcaattgccgtggaggacgcacacatttgtgccgtaacactacaggcgtaggcgtgaatcgtcccggatgtttcgcggaataccttgtcattccagcgtttaacgcctttaagatccctgacaacatttcagatgatttagcatccatttttgacccattcggtaacgcggtccatactgcgttgagcttcgacttagttggagaagatgtattagtttccggcgccggaccgattggcgtcatggcagctgccgttgcgaagcacgtgggcgcacgtcatgtggtaattacggacgtaaatgagtatcgtctggagctggcacgtaaaatgggggttacacgtgccgtaaacgttgcgaaagagtctttaaacgatgtcatggctgaactgggcatgacggaagggtttgatgtcggactggaaatgtccggtgccccgccagccttccgtaccatgttggacaccatgaaccatgggggccgtatcgcaatgttgggaattcccccgagcgacatgtctatcgactggacaaaggtaatttttaaaggcctgttcattaaggggatttacggtcgtgagatgtttgagacgtggtacaagatggctgccttgattcaatcggggttggatctgagccctatcatcacacaccgtttttcagtggatgactttcaaaaagggtttgacgccatgtgcagcggtcaatcagggaaagtaattctttcttgggac

SEQ ID NO: 67 - DNA, 999 bp, Escherichia coli
  misc_feature 1..999: note = threonine aldolase (Escherichia coliO26:H11 str. CVM10026)
  source 1..999: mol_type = genomic DNA
atgattgaccttcgttcggacaccgtaacccgcccatctcacgcaatgttggaagctatgatggccgcgcctgtgggggatgacgtttatggggatgacccgaccgtcaacgctttacaagattacgctgctgaattgtcgggcaaagaagcagcaatcttcttacctacaggtacacaagctaatcttgtcgccctgcttagtcactgtgagcgtggcgaagaatacattgttggtcaagcagcgcataattacctgttcgaagctggaggggctgctgttcttggtagcattcagccccaacccattgatgctgctgccgatggtactcttcctctggataaagtcgctatgaaaattaagccagacgacattcacttcgcacgcacaaagctgctgtcgcttgagaatacacacaatggaaaagtcctgccccgtgagtacctgaaagaggcttgggaatttacacgcgaacgcaacctggctctgcacgtagacggtgctcgcatcttcaacgccgttgtcgcctacggttgcgaattgaaagagattacgcaatactgtgactccttcacgatttgcttgtccaaaggcttaggcaccccggtgggttcattgttggtaggaaaccgtgactatattaagcgcgccatccgctggcgtaaaatggcagggggtggaatgcgtcaatcagggattcttgcggcagctggcatgtacgcgctgaaaaataatgtggctcgccttcaagaggatcacgataatgctgcgtggatggctgagcaattacgtgaggcgggtgcagacgtaatgcgccaagataccaatatgctgttcgtacgtgttggggaagaaaacgctgcggccttaggagaatacatgaaggcgcgtaacgtgttgatcaacgcatcccctattgttcgccttgtaactcaccttgatgtttcacgtgaacaattggcggaagttgccgcccactggcgtgcctttcttgctcgc

SEQ ID NO: 68 - DNA, 1251 bp, Escherichia coli
  misc_feature 1..1251: note = serine hydroxymethyltransferase (Escherichia coli)
  source 1..1251: mol_type = genomic DNA
atgcttaaacgtgagatgaatatcgccgactacgacgccgaattatggcaggcgatggagcaggagaaagtccgccaagaggaacacattgagcttattgcgtcggagaactatacatcccctcgcgttatgcaggcgcaaggctcacagttgacgaacaaatacgctgagggatatccgggaaagcgttattatggcggttgcgagtacgttgacattgttgaacagttagcgattgatcgtgctaaggagttatttggagcggattatgccaatgttcaacctcactcgggcagccaggctaactttgctgtatacaccgcacttttagaacctggtgacacggtcctgggtatgaatttggcccatggaggccacttaactcatggaagccctgtgaattttagtgggaagttgtataacatcgtgccctacgggatcgacgccacaggacacattgattacgcagatttggagaaacaagccaaggaacataagcctaaaatgatcatcggcggattttcagcatatagcggagtggtagactgggccaaaatgcgcgagattgctgattcgattggtgcttacctgtttgtcgatatggcgcatgtcgctggtctggtcgctgcgggagtttatcctaaccccgtgcctcacgctcacgtcgtgacgactactacacataagactttagcgggtcctcgtgggggtttgattcttgcgaaggggggctcagaggaactttataagaagcttaactctgccgtatttcccggcggtcaggggggccctcttatgcacgtcatcgcaggaaaggcggtggctctgaaggaagcgatggaacccgaattcaagacttaccaacagcaagtagccaaaaacgccaaagccatggtggaggtattcctggagcgcggctacaaggtagttagcggggggacggacaaccatttgttcttagtcgatttagtggacaaaaaccttactggtaaggaggctgatgctgctcttgggcgtgcaaatatcacagtcaataagaatagcgtgcccaatgacccaaagtcgccatttgtgacttctggcatccgcgttgggactccggcaatcacccgtcgtggctttaaggaggcagaggccaaggagctggcagggtggatgtgtgacgtactggactctattaatgatgaggcagttatcgaacgtattaaaggcaaagtgcttgacatttgtgcgcgctaccccgtgtatgcc

SEQ ID NO: 69 - DNA, 1293 bp, Escherichia coli
  misc_feature 1..1293: note = tdcC(Escherichia coli)
  source 1..1293: mol_type = genomic DNA
atgtctacttcggactctattgtttcatcgcaaacaaaacagtcatcctggcgtaaatcagataccacctggactttgggtctgtttggtaccgcgatcggggctggtgtattgtttttcccgatccgcgctggatttggtggtttaattcctatcctgctgatgcttgtactggcatatcctattgctttttattgtcatcgcgcagcgcgcttgtgtttaagcggaagcaacccctcgggtaatatcacagagacggtggaggagcatttcgggaaaacaggaggggtcgtaatcacatttctgtacttttttgctatttgtcccctgttgtggatttatggggttacgatcaccaatacttttatgacgttttgggagaatcaactgggctttgcaccgcttaaccgcggattcgtggcgctgttccttttactgttgatggcgtttgtcatctggttcggtaaagacttaatggtgaaagtcatgtcttatttggtatggcctttcattgcttcacttgtcttaattagtctgtcattaatcccttattggaactcggcagtaatcgatcaagtagatctgggtagcctgtctttgaccggacatgatgggatcttaattaccgtatggctgggcatttctattatggtctttagttttaacttttcacctatcgtgtcctcctttgtggtgtccaagcgcgaggaatatgagaaggattttggtcgtgattttacggaacgtaagtgctcacaaattattagccgcgcgtctatgcttatggtggctgtcgttatgttctttgctttctcctgcttatttaccttgtcaccggcgaacatggcggaagcgaaggcgcaaaacattccagttttatcatatcttgctaatcatttcgcttctatgacagggaccaaaactacttttgccatcacattggagtatgcggcgtctatcattgcattagtggccatttttaagtcgttctttggccattatttaggtactttagaagggttgaatggcttagtcttgaaattcggatacaagggggacaaaactaaagtttccttgggtaagttgaacacaatctcgatgatctttattatggggagtacatgggtcgttgcgtatgcaaatccaaacattctggatttaattgaggcgatgggagcaccgattatcgcgtcattgttgtgccttttgccgatgtacgccatccgtaaggcgccttcactggccaaatatcgtgggcgcttggataacgtgttcgtaaccgtcatcgtttgc

SEQ ID NO: 70 - DNA, 885 bp, Escherichia coli
  misc_feature 1..885: note = Threonine/ homoserine exporterRhtA, Escherichia coli (strain K12)
  source 1..885: mol_type = genomic DNA
atgcctggttccttgcgtaaaatgccggtttggttgccgattgttattcttctggttgcaatggctagcatccaaggaggcgctagtttagcaaaaagtctgtttcctttggtgggggcaccgggtgtgaccgcgctgcgtttggctttgggcactttaattttgattgccttctttaagccctggcgccttcgttttgctaaagaacaacgtttgccgcttttgttctacggcgtctcacttggtggcatgaactatcttttttatttaagcatccaaaccgtacccctgggtattgcggtggctttggagttcacgggtccattggcagttgcccttttcagctcgcgtcgcccagtcgatttcgtctgggtagtgcttgcggtacttggactgtggttcttactgcccttaggccaagacgtgagtcacgtagaccttaccgggtgtgcgctggctttgggagccggtgcttgttgggcaatttacatcctgtcgggacagcgtgcgggagcagagcacgggcctgcgacagtagcgattgggtcgctgatcgcagccctgattttcgtccccattggtgccttacaggcaggagaggcgttgtggcactggtcagtgattcccttaggtttggcggtagcaatcctgtctaccgcacttccttattctttagagatgattgccttaacccgtctgccgacacgtacgtttggcaccttaatgtcgatggaaccggcattggctgccgtttcaggtatgatcttcctgggagagacgttaactcccattcagttgttagctcttggggcaatcatcgctgcgagtatgggatcgacccttacggttcgtaaagagtcgaagattaaagaattggacatcaat

SEQ ID NO: 71 - DNA, 618 bp, Escherichia coli
  misc_feature 1..618: note = rhtB(Escherichia coliFVEC1302)
  source 1..618: mol_type = genomic DNA
atgacgctggagtggtggttcgcatacttgctgacatccatcatcctgagtttaagccccggatctggtgcaatcaacacgatgactacgtctttgaatcacggctatcgtggtgctgttgcatccattgccggcttgcagacgggattagccatccatattgttttagtgggtgtaggacttggaacattattcagtcgctcggttatcgcctttgaggtcttaaagtgggctggtgccgcttatttgatttggctgggaattcagcaatggcgtgcagccggtgcgattgacttgaagagccttgcgtccacacagagccgccgtcacttgtttcaacgtgcagtattcgtcaatttgaccaaccccaaaagtatcgtctttctggcggcactgtttccccagttcattatgcctcaacagccgcagttgatgcagtacatcgtcttgggcgtcaccaccatcgtagtggacattattgtaatgattggatacgccactctggcccaacgtattgcgctgtggatcaagggcccgaaacagatgaaggcactgaacaaaatttttggttctttgtttatgttggttggggcacttcttgccagtgcacgtcacgcg

SEQ ID NO: 72 - DNA, 618 bp, Escherichia coli
  misc_feature 1..618: note = RhtCthreonine Rht Transporter (Escherichia coliBL21(DE3))
  source 1..618: mol_type = genomic DNA
atgctgatgctttttttaacagtagcaatggtgcatatcgtcgcattgatgtcaccgggacctgactttttttttgtttcacaaacagcagtatcacgctcacgtaaggaggcaatgatgggtgtcttagggatcacttgcggcgtaatggtatgggccggtattgcacttctgggactgcatttaattattgagaagatggcctggcttcacacattaatcatggtaggcggtgggctttatttatgttggatgggctatcaaatgctgcgtggagctcttaagaaagaagccgtgtccgcaccggctccccaagtggaacttgcgaaatcaggtcgctccttcttgaaggggttgttgactaatcttgcgaaccctaaggccatcatttatttcggttctgtgtttagtttgttcgttggggataatgtgggaaccacggaacgctggggaatcttcgcattaatcattatcgagacgttagcttggttcaccgtcgtggcctccctttttgctctgccgcaaatgcgccgtggttaccaacgtttagcaaagtggatcgacggttttgctggagctttatttgcgggtttcggcattcatctgattattagccgt

SEQ ID NO: 73 - DNA, 984 bp, Escherichia coli
  misc_feature 1..984: note = cysteine desulfhydrase (Escherichia coli)
  source 1..984: mol_type = genomic DNA
atgcccctgcacaacttaacacgttttccacgcctggaattcattggtgcaccgactcccttggaatatctgcctcgcttttcggactacttaggccgcgagattttcattaagcgcgatgatgttacaccgatggctatggggggtaacaaattgcgtaaattggaatttcttgcagcggatgcactgcgtgaaggcgcggacactttaattaccgctggtgcaattcagtcaaatcacgtacgccaaactgcggcagttgctgcgaagttaggtcttcattgtgtcgcccttttggaaaatccaattggcacaacggcagaaaattaccttaccaacgggaaccgtttgttgcttgacctttttaacacacagatcgaaatgtgcgacgctttaactgatcccaacgctcaattggaggagcttgcgactcgcgtggaagctcaaggcttccgtccgtatgttattccggtcggcggcagcaatgctcttggggcattagggtatgtagagtccgctctggagatcgcgcaacaatgtgagggcgcggttaacatttcgagtgtagttgtggcctctggaagtgcgggcacccacgccgggctggctgtgggtcttgagcacttaatgcctgaatctgaactgatcggggtcacagtctcgcgttccgtcgcagatcagttacctaaggtagtaaacttacagcaagccattgcgaaagaattagaattaaccgctagtgcagaaatcttattatgggatgattactttgcgcctgggtacggtgtccccaatgatgaaggtatggaagcagtcaagcttttagctcgtttggaggggatcttgctggaccctgtttacaccggcaaagcaatggcaggcttaattgacggtatcagtcagaaacgcttcaaagacgagggaccaattctgttcatccataccggcggcgctcctgccctttttgcctaccaccctcacgtt

SEQ ID NO: 74 - DNA, 1413 bp, Escherichia coli
  misc_feature 1..1413: note = tnaA (Escherichia coliDH1)
  source 1..1413: mol_type = genomic DNA
atggagaatttcaagcatttgcccgagccgttccgcattcgtgtcattgagcctgtcaagcgtactactcgcgcgtatcgcgaagaggcgattatcaaatcgggtatgaatccatttttacttgattcagaagatgtgttcatcgatttacttacagattctgggacaggcgcggtaacgcaatcgatgcaagcagcgatgatgcgcggtgacgaagcctattctggctcgcgctcctattatgctctggccgaatcagtcaaaaacatttttggttaccaatatacgattcccacgcatcagggacgcggagcagagcaaatctatatcccagtcttaatcaaaaagcgcgagcaagaaaagggattggaccgctcgaaaatggtagccttctcaaattacttcttcgacactactcaggggcactcgcaaatcaacggctgcactgttcgcaatgtgtatatcaaggaagcctttgatacaggcgtacgttacgatttcaaggggaactttgacctggaaggtcttgaacgtggcattgaagaagtaggacccaacaacgtaccctatatcgtcgccacgatcacatctaatagcgcaggaggtcagcctgtgtctttggcgaatctgaaagcgatgtattcgatcgccaaaaagtatgatatccccgtcgtaatggattctgcacgttttgcagagaacgcctacttcattaaacagcgtgaagcggagtacaaagattggaccatcgaacagatcactcgtgagacttataaatatgctgacatgctggctatgtcggctaagaaggacgctatggtcccaatgggaggccttttatgcatgaaggacgatagtttttttgacgtttatacggaatgtcgcaccctttgtgtagtgcaggaaggattccccacttatggcggccttgaaggtggagcgatggaacgtttagctgttggactgtatgatggtatgaatctggattggctggcatatcgtattgcgcaggtgcagtacctggtagacgggttagaggagatcggggttgtgtgccagcaggccgggggccatgcggcgttcgtggacgcaggaaaactgcttccccacattcccgccgatcagttccctgcgcaggcacttgcttgcgagttatacaaggtggccggtatccgtgcggtagagatcggctcgtttcttttggggcgcgaccctaaaacaggaaaacaattgccctgccctgccgaacttcttcgccttactatccctcgtgcgacctacactcaaacccacatggactttattatcgaggccttcaaacatgtgaaggagaatgctgctaatatcaagggcctgacctttacctacgagccaaaggttttgcgccactttacagcaaaacttaaagaagtt

SEQ ID NO: 75 - DNA, 969 bp, Escherichia coli
  misc_feature 1..969: note = cysK (Escherichia coliO104:H4 str. C227-11)
  source 1..969: mol_type = genomic DNA
atgtcaaaaattttcgaggataactcgttaacgatcggccacactcccttggttcgtctgaatcgtatcggtaacgggcgcattctggcaaaggttgaatcacgcaatccgtccttctcagttaagtgccgtattggagcgaatatgatttgggatgctgagaagcgcggagtcctgaagcctggggtggagttggtggagccaacctctgggaatacaggtatcgcgctggcttatgtagctgcagcgcgtggctacaaattaacacttaccatgcccgagaccatgtcaatcgaacgtcgtaagttgttgaaggcattaggagcgaatctggtactgaccgaaggagctaagggaatgaagggcgctattcaaaaagcggaagaaattgtcgcaagtaaccccgaaaagtatcttttactgcaacagttttctaaccctgcaaatcctgagatccacgaaaaaacaacaggtcccgaaatctgggaagacaccgacggtcaagttgacgtatttatcgccggggtaggaactggaggaaccttaacgggggtcagtcgttatattaagggtacgaagggaaagactgatttgattagcgtagcagtggagccaacggatagtcctgttattgcccaagccctggcgggggaggaaatcaaaccgggacctcacaaaatccaagggattggtgcgggttttatcccagccaatctggatctgaaacttgtcgacaaggtcattggaattactaatgaagaggcgatctccactgcgcgccgtttgatggaggaagaagggattttggcagggatttcaagcggtgcggcggtggcagcagctttgaaattgcaagaagacgagtcattcactaataagaatattgttgttattttaccaagcagcggtgagcgctacttatcaaccgctttgttcgctgatttatttacggaaaaagagttacaacaa

SEQ ID NO: 76 - DNA, 909 bp, Escherichia coli
  misc_feature 1..909: note = cysM (Escherichia coliFVEC1412)
  source 1..909: mol_type = genomic DNA
atgtcaacattagaacagacaattggtaatacccccctggtcaaattgcagcgcatggggccaaacaatggaagcgaggtttggctgaaattggaaggcaacaacccggcgggatctgtgaaagaccgtgccgcactgtccatgatcgtagaagctgagaaacgtggcgagattaaacctggggatgttttaatcgaggctacaagtgggaacactggaatcgcccttgccatgattgcggctttaaagggttatcgtatgaagttacttatgcccgataacatgagccaggagcgccgtgccgctatgcgtgcctatggtgctgaacttatcttagttaccaaggagcaaggcatggaaggtgcgcgtgacttggcattagaaatggcgaatcgtggcgaagggaagctgcttgaccaatttaataatccagataacccttatgcacactataccacgaccggcccggaaatctggcaacaaaccggcgggcgcatcacccactttgtatcatccatgggcacaactggtacaattacgggcgtttctcgtttcatgcgcgagcagagtaaacctgttacaatcgtgggacttcaacctgaggagggatcttcgatcccaggcattcgtcgttggcctgctgagtacttacctggcattttcaacgcatccttagtggatgaagttcttgacattcatcagcgcgaagcagagaataccatgcgcgagttggcagtacgtgagggcattttctgcggggtttcttctgggggggccgtggcgggtgctttacgtgtcgccaaagcaaaccccggagcagtagttgttgccattatttgtgatcgtggtgaccgctacttatctacgggagtcttcggagaggaacacttttcacaaggggccggaatt

SEQ ID NO: 77 - DNA, 1170 bp, Escherichia coli
  misc_feature 1..1170: note = malY (Escherichia coli)
  source 1..1170: mol_type = genomic DNA
atgttcgatttttcgaaagtcgtcgatcgtcatgggacctggtgcactcaatgggactacgtggcggaccgctttgggacagcagatttgttaccgttcactattagcgacatggattttgccacagcaccttgcattatcgaggcactgaatcagcgcttaatgcatggggttttcggttatagccgttggaagaacgatgagttccttgcagcaattgcacattggttcagtacccaacattataccgctatcgattcccagacggttgtgtacggccccagcgttatttacatggtgagcgaattgatccgtcagtggtctgaaacaggagaaggtgtagtaatccatactcccgcctatgacgcgttctacaaagccattgaggggaatcaacgtacagtaatgcccgttgccttagaaaaacaggcagacggatggttttgcgatatgggaaaattagaggcggtacttgcaaaacccgagtgcaaaatcatgcttttatgcagtccgcaaaacccaacaggcaaggtctggacctgtgatgaattagagattatggcggatttgtgcgagcgtcacggagtccgtgtcatctctgacgagattcacatggacatggtctggggggaacagccgcacattccttggtctaatgtcgcacgtggtgattgggcccttttgacatcgggttcgaaaagctttaacattccagccctgaccggggcatatggaattatcgaaaactcgtcgagccgtgacgcgtatttatctgcccttaagggacgtgatggactttcgagcccgtcggttcttgccttgacggcacacattgctgcttaccaacagggagcgccgtggctggacgctcttcgcatttacctgaaggataaccttacttacattgcggataagatgaatgcggccttcccagaacttaactggcagattccccagtcaacgtatttagcctggcttgaccttcgtcccttaaacattgatgacaacgcactgcaaaaggcactgatcgaacaggaaaaggtagccatcatgcctggctatacctacggcgaggagggccgtgggttcgtccgcctgaacgcaggatgtccccgctcgaaacttgaaaaaggggtagctggtcttattaatgctattcgcgctgtgcgc

SEQ ID NO: 78 - DNA, 1185 bp, Escherichia coli
  misc_feature 1..1185: note = MetC (Escherichia coli)
  source 1..1185: mol_type = genomic DNA
atggccgacaagaagttggatactcaactggtgaacgccgggcgttccaaaaaatataccttgggagctgttaatagcgttatccaacgtgcatcaagtttagttttcgatagtgtcgaagcaaagaagcatgcgacacgcaatcgcgcaaatggggaattattttatggacgccgcgggaccttgacccacttctctttacagcaggccatgtgtgagctggaagggggagccggttgtgtattgttcccctgcggagccgcggcggtggctaacagtatcctggcgttcgtggagcagggtgatcacgtcctgatgacgaacaccgcgtacgaaccctcgcaagacttctgcagtaaaatcttatccaaattaggtgtgactacctcgtggtttgacccgttgatcggggcggacattgtgaaacatctgcagcccaacacgaaaattgtttttttggagtctcccggttcgattactatggaggtacacgacgtgccagctatcgttgcagcagttcgttccgtggcgcccgacgcaattatcatgatcgacaatacatgggccgcaggcgtcctttttaaagccttagattttggcattgatgtaagtatccaagcggctaccaagtacttggtcggacattccgatgcgatgattggtacagcagtatgcaatgcacgctgctgggagcaattgcgtgaaaacgcttacctgatggggcaaatggtagacgcagataccgcttatattaccagtcgtgggttgcgtacattaggagtgcgtttgcgtcaacaccacgagtcatccctgaaagtggctgaatggctggctgaacatccccaggttgctcgcgtaaaccaccccgcacttccgggatcaaagggccatgaattttggaagcgcgacttcacgggctccagtggattgttttctttcgtacttaagaaaaagttgtctaatgaagaattggcgaattaccttgataactttagcttgtttagtatggcatatagttgggggggatatgaatcactgattttggcaaatcaaccagaacatattgctgcgattcgtcctcaaggcgaaattgattttagcggaacgttaattcgtctgcacatcgggcttgaggatgtggacgatttaattgcagatttggatgcgggatttgcacgtattgtg

SEQ ID NO: 79 - DNA, 1230 bp, Trypanosoma grayi
  misc_feature 1..1230: note = cystathione gamma lyase (Trypanosoma grayi)
  source 1..1230: mol_type = genomic DNA
atgtcaggtgcccagcacttgttcgcagatttcagcgaaggatcaggatcgtggcaaccccaggcccaagggtttgagacgcttctggtacatggtggcgtaaagccagatcccgtcacgggggcaatcctgacccccgtctaccagtctacgacgttcgtgcaagagagtatcgaacgttatcaagcaaagggctatagctatacccgttcagccaatcctaccgtatctgcattggaagagaaattgtgcgcaatcgagcacggcgaatatgccactgtgtatagcaccggcatgtccgctacgacaacggccatcagtagttttatgtctgctggcgaccacgctattgtgaccgaatgtagctatggcggaaccaatcgtgcctgccgtgtcttcttcacgcgcttaggtatgtcttttacattcgtagatatgcgcgacgttaaaaatgtagaggctgccatcaaacccaataccaagctggttatctcagaatcgccagcaaaccctacactgacgcttactgatattgacgcacttagctcgctttgcaaggctaagggtattattcacatgtgtgacaacactttcgcaaccgctttcattatgcgtccgcttgatcacggagcagacgtgaccctgatctccacgactaagtttgttgatggccacaatatgaccgtcggaggggccttggtcactaaatccaaggaattagacggaaaggtacgtttaacgcaaaatatcttaggtaactgtatgagtccatttgttgcgttccttcaattacaaacggtgaagacgatgagccttcgcatttctcgtcaatcagaaaacgcccagaaagtagcggaatttcttgagacccaccccgcagtggaacgcgtaatgtatccaggtcttaaatctttcccacagaaggccttagcggatcgtcagcacgcaaacaatttacatggcggtatgttatggtttgaagtgcgcggaggaacagcggcagggcgtcgcttgatggacaccgttcagcgcccgtggagcttatgcgagaatctgggtgcgacggaatccatcattacttgcccgagtgtcatgacccacgcgaacatgactactgaggaccgtatgaaggtcggtatcaccgacggatttgtacgtgtcagctgcgggatcgaagatgcagccgatcttatctcagctttgaaggccgcactggatgccttgggcaag

SEQ ID NO: 80 - DNA, 915 bp, Helicobacter pylori
  misc_feature 1..915: note = Cystathione beta-synthase (Helicobacter pylori 2017)
  source 1..915: mol_type = genomic DNA
atgatcttaacagcaatgcaagatgcaatcgggcgtacacctatcttcaagtttacacgtaaagattacccaattccattgaagtcggcaatttacgcgaaattggaacacttaaacccggggggatccgtgaaagatcgccttgggcagtatcttattaaggaggccttccgtacacacaagattacctctactaccactatcatcgaacctactgctgggaatactggcatcgcccttgcccttgtagctatcaaacatcatcttaaaacgatctttgttgttcccgaaaaattttcggttgagaaacaacagatcatgcgtgctcttggtgccttagtaatcaatacgcctacctcagagggtatctcaggggccattaaaaaaagcaaagagttagccgagtctatcccggacagctacttgcctcttcaatttgagaatcccgacaatccggctgcttattaccacactcttgctcctgaaattgtaaaggaactggggacgaattttacctcttttgtagcgggcatcggttctggaggaactttcgcaggcaccgccaagtaccttaaagaacgtatcccgaacatccgcttgattggagttgaaccagaaggttctattttaaatgggggtgaaccggggccccacgaaatcgaaggaattggagtagagttcatcccaccattcttcgctaatttggatattgatgggtttgagacgatttcagacgaagagggcttcagttatacgcgcaaattagccaaaaagaacggattattagtgggtagttcgtccggagcagcgttcgccgcggctcttaaggaagtacaacgtctgcccgaagggtcacaagtgttgacgattttcccagatatggctgatcgctaccttagtaaaggcatttattcc

SEQ ID NO: 81 - DNA, 1320 bp, Helicobacter pylori
  misc_feature 1..1320: note = putativeamino transferase (Helicobacter pylori 2017)
  source 1..1320: mol_type = genomic DNA
atgcaagctttcttgaaccgttcgttcgcgccccttttaaacccaaatgagaacctgctggatcaagttaagagttcgattattttgaagaaaggtgttagctactttgactggggtgctagtgggctggccagtgcattggtcgagaaacgtgttaagtccctgcttccatattatgccaatgcccacagcgtagcaagtaaacatgccatcttaatgggcatgttacttaaagaatgccaagagaagctgaaacgctcgttaaaccttagtactaaccattgcgtgcttagcgccgggtatggcgcgagctcagcgatcaagaaattccaagagatcctgggagtttgcatcccctctaaaaccaaaaagaatctggaaccttatttaaaagacatggcgctgaaacgcgtaatcgtaggtccttatgaacatcactctaacgaggtctcttggcgcgagtctctttgtgaggtggtgcgcattccacttaacgaacatggactgctggatttggagattttagagcagatcttaaagaaatcccccaattctctggtctccgtctcggccgcaagtaatgtaacggggattctgacacccctgaaagaaattagctcactgtgcaaggagtatcgcgcgatcctggcgcttgatctggccaacttttccgcacacgcgaacccgaaagactgcgagtaccagacggggttctatgcaccacacaagttgttgggtggtattgggggatgcgggcttcttggaatctccaaagacttgatcgatacacagatcccacctagtttttcagccggaggagtcattaagtacgcaaaccgcacgcgtcacgaatttattgatgagctgccgttgcgtgaggagttcggaactccgggactgctgcaattttatcgctcagtgttagcctaccagttacgtgacgaatgcggtttggatttcattcataagaaggagaataatctgcttcgtgtgttaatgcatggcttgaaagatctgccagctatcaacatttacggcaatttaaccgcaagccgcgtaggagtagtcgcgtttaacatcggaggcattagtccatacgatcttgcccgtgtcctgagttacgaatatgctattgagactcgcgcagggtgctcttgtgccggcccgtatggacatgacttactgaatttgaatgcacaaaagtcttccgatttcaatgcaaaacctggatggttgcgcgtctcacttcattttacacacagtattaatgacattgactatctgttggactctctgaagaaagctgttaagaaactgcgt

SEQ ID NO: 82 - DNA, 918 bp, Bacillus atrophaeus
  misc_feature 1..918: note = YdeD (Bacillus atrophaeus UCMB-5137)
  source 1..918: mol_type = genomic DNA
atgaacggtgaacacgccgcgttggcccactcccgcacaaaagggattgctttggttttaacgggcagtatcttatggggcgtttcagggacagttgcgcagtacttattccaacaacaacattttaacgtagagtggttgaccgtcgttcgcttgttgctgtctggtatcttgctgcttggccttgcctatcgtaaggaaaagcaacgcatctgggctgtctggaaagacaagacagatggtctgaatctggttctgttcgggattttggggatgttgtccgtccagtacacatactttgcggctatccagcatggtaatgcggcgacggcaactgtacttcagtatctggccccggcacttattacctgctacgtagccattcgctctaagcgtcttccaaccgtcaaagagttgatcgcagttttcctggctattattggaacgttttttttagtcacccatggggacatccacagtcttagtatctcagggtgggctttattctggggattaagttcggcgtttgccctggcgttttacactttgcaccctcataaacttctggccaagtggggggcggctatcgttgttggctggggtatgcttatcggagggcttggtctttccttaatccatcctccatggaaatttgagggacagtggtcggtctcggcttatgccgccgttattttcattgtcctgtttgggaccctgactgccttctactgctacctggaatctttaaagtacttaactgccagcgaaacttcattaatcgcctgcgcggagcccttaagtgctgcgttcttaagcgtgatttggttgcatgtgacttttggtatcagcgagtggcttggtacttgttgtattttatctacgattatgatcttatcgattaaggagaagaagctgaag

SEQ ID NO: 83 - DNA, 585 bp, Escherichia coli
  misc_feature 1..585: note = proteinYfiK (Escherichia coli)
  source 1..585: mol_type = genomic DNA
atgacacccacgttgcttagcgccttctggacgtacacccttattacagccatgacgcctgggccaaataatatccttgccttatcatccgcaacgtcgcatgggttccgccagtccacccgtgtgcttgcaggtatgtctcttggctttttaatcgttatgctgttgtgcgcgggaatcagtttctccttggcggtaatcgaccccgccgccgtacatttattgtcttgggctggtgccgcgtatattgtttggctggcttggaaaattgccacgtctccgactaaggaagatggtttacaagcaaaacccatctcgttttgggcttcatttgcacttcagttcgtgaatgtcaagattattctttacggggtaacagccctgtccactttcgttttaccccagacgcaggcgttgtcatgggtagtcggagtgtccgtcttattagccatgatcggtacgtttgggaatgtgtgctgggcgctggcgggccacttgtttcaacaattattccgtcagtacggtcgccagttaaatatcgttcttgctttattactggtgtattgtgcagtccgcatcttctat

SEQ ID NO: 84 - DNA, 1188 bp, Escherichia coli
  misc_feature 1..1188: note = multidrug efflux transporterBcr(Escherichia coli)
  source 1..1188: mol_type = genomic DNA
atgacgacccgccagcatagctcgttcgcaatcgtatttattcttggattgcttgctatgttgatgccattatcaatcgacatgtacttaccagccctgcctgttatttcggcccaatttggagtacccgctgggtcaacccaaatgacattatcaacatacattctggggttcgctttaggacagttgatttatggtccaatggctgactcgtttgggcgcaaaccagtggtcttgggcgggacactggtctttgcggccgcagccgttgcgtgtgccttggctaacacgatcgaccagcttattgtaatgcgtttcttccatggcttagctgcggcggctgccagtgtagtgattaatgcgcttatgcgtgacatctatccgaaggaggaattcagccgcatgatgagcttcgtaatgttggtaacgaccatcgctccattaatggcccctattgttgggggttgggtcttagtctggctttcatggcattacattttttggatccttgccctggcggctattctggcctcagcgatgattttcttcctgattaaagaaacccttcctccggagcgccgtcagcctttccatattcgcactactatcggtaattttgcggccttgtttcgccataaacgcgtgctgtcatacatgttggcaagcggcttttctttcgcgggtatgttctcgtttttaagtgctggtcccttcgtgtatatcgaaatcaatcacgtagccccggagaacttcggctattacttcgcattaaatatcgtgtttcttttcgtcatgaccatcttcaactctcgcttcgtccgtcgtatcggtgccttaaatatgtttcgttcggggctgtggatccaatttatcatggctgcgtggatggtgatctccgcactgttggggcttgggttttggtcgcttgtggtgggcgtggctgcattcgttggatgtgtcagcatggtatcttctaacgcgatggctgtaattttggatgagttcccacatatggcagggactgcttcctctctggctggcacatttcgcttcggaattggtgcaatcgtaggcgcgttgctgagcttagcgacattcaattcggcgtggcccatgatttggtccattgcgttttgtgcgaccagcagcatcctgttctgcctttatgcttcccgtccaaagaagcgt

SEQ ID NO: 85 - DNA, 1356 bp, Pseudomonas fluorescens
  misc_feature 1..1356: note = TolC (Pseudomonas fluorescens R124)
  source 1..1356: mol_type = genomic DNA
atgaacaaacttagtatgctgggagctgccttcgcgttgttggcagggaactcagcattggcagcaatggggcctttcgaaatctacgaacaggctcttcgcaatgacccagttttcttaggggccattaaggagcgtgacgccggattggaaaaccgcatcatcggccgcgcaggattgttaccacgcttggggtacaactacaatcgtggccataacacctctaaagcgacccagttgacaaatcgtggctctctgactgaagaccgtaactataattcgtatggttcaactcttacattacagcaacccttattagactatgaggcctatgccgcctaccgtaagggagtagcgcaaagcttgttcgccgatgaagcctttcgcggtaagtcacaggaattattggttcgcgtcttagataattacacgaaagcgttgttcgcacaagaccaaatcgatatcgcacaggcgaaaaaaaaagcttatgaacaacaatttcagcagaacgaacatatgttcaaacaaggcgaggggacgcgcactgacattttggaagctgaaagtcgttatgaacttgccacggcagaagaaatcgaggcgcgtaacgaacaggatgccgctcttcgcgagcttggtgcgcttgtcggtgtcccaactgtcgacatttctgaacttgcacccttagaccagaattttcaaacgttcgcgctgatgcctgctaactatgatacgtggcacgagttagcaatttctaataatccgaacctggcatcacagcgtcaggccgtggaagtagcaaaatacgaagttgaacgtaaccgtgcaggacatttacccaaggtctcagcatatgccagcattcgtcagactgagtctgacagtggtaatacctacaatcaacgttatgatacgaacaccattggctttgaggtaaacgtccctctgtatgcaggaggaggagtctcagcaagtacacgccaagcatcacgcacgatggagcaggcggagtatgaattagatggaaagacgcgtgagacgttaattgaattacgtcgtcagttcagcgcgtgccttagtggagttaataagttacgcgcctatcagaaagccctggcctcggccgaagcactggtggtctcaaccaagcagagcattcttggcggcgaacgcaccaacttggacgcgcttaacgcggaacagcagctgttcaccacgcgtcgcgaccttgcacaggcccgctatgactacttgatggcgtggacgaaactgcattattacgcaggaaccctgaacgaacaagatttagcgcgtgtggacgaggcatttggccaagggcccaaatcaaatcctcgc

SEQ ID NO: 86 - DNA, 1167 bp, Sinorhizobium meliloti
  misc_feature 1..1167: note = Tyrosine transaminase (Sinorhizobium meliloti AK83)
  source 1..1167: mol_type = genomic DNA
atgttcgatgcgctggcgcgtcaagcggatgatccgcttttggcgctgatcggactgtttcgcaaagacgagcgccccggtaaagtggacttaggtgtgggagtttaccgcgacgaaactggccgcactccgatctttcgcgcggttaaagcagccgaaaaacgcttgcttgagactcaggactcgaaggcctacatcggcccggaaggagacctggtttttcttgaccgtttgtgggaacttgttgggggggataccattgaacgttctcacgtagctggtgtacaaacacctggcgggagcggcgcacttcgtttggcggcagatttaatcgcccgcatgggcggtcgcgggatttggttggggttgccatcctggccgaatcacgctcccattttcaaagcggctggactggatatcgcgacttacgatttctttgatatcccgagtcaatccgttatttttgataacctggtgtctgccctggaaggtgcagcatctggcgatgccgtcttattgcatgctagctgccacaatccaactggaggggtattatccgaggcacagtggatggaaattgccgcgctggtcgccgaacgcggactgttaccacttgttgatcttgcgtatcaagggttcggacgtgggctggatcaagacgtcgcgggcttacgccatttattaggtgtagttcccgaagcccttgtcgccgttagctgctctaaatcgttcggcttgtaccgcgaacgcgctggagccatcttcgcccgtacatcatctaccgcttcagccgaccgcgtccgcagtaacttagctggccttgctcgcacatcgtatagtatgccccccgatcacggggccgcggttgtccgtacgatcttagacgacccagagctgcgtcgtgactggaccgaggaattagagacaatgcgcttgcgtatgacgggtcttcgccgctctcttgcagagggcttgcgcacccgttggcagtctcttggcgccgtagctgaccaagaagggatgttctcgatgctgccgttgtccgaagcagaggttatgcgccttcgcactgagcatggaatttacatgcccgcatcaggacgcattaacattgcggggttaaaaacggcggaggctgccgaaattgcaggtaaatttacgagtttg

SEQ ID NO: 87 - DNA, 1209 bp, Escherichia coli
  misc_feature 1..1209: note = tyrosine transporterTyrP(Escherichia coliW)
  source 1..1209: mol_type = genomic DNA
atgaagaaccgcactcttggatcagtattcattgttgcggggaccaccatcggtgcaggtatgcttgccatgcccctggctgcagctggcgtcgggttcagcgttaccctgattttactgattggtctgtgggctctgatgtgttacacggcattgcttttgcttgaagtgtaccagcatgtacccgcagacaccggtcttggcactctggcgaaacgttatttaggacgttatggtcaatggctgaccggtttctccatgatgtttctgatgtatgcgctgacggccgcatacattagtggtgcaggtgaactgctggcaagttcaatttctgactggacgggcatctctatgagcgcgactgctggggttttattgtttacatttgtggctggcggtgtagtgtgtgtagggacgtcattagttgatctgtttaaccgcttccttttcagtgcaaaaatcattttccttgtagtaatgcttgtcttattattaccacatattcataaggtaaatcttttgacattaccattgcagcagggattggcgttatcagccatccctgtaatcttcacatccttcggattccacgggtccgtcccatccatcgtgtcctacatggacggcaatgtacgcaagttacgttgggtctttatcacagggagcgccattccccttgtagcgtatattttttggcaagttgctactctggggtcaatcgactctaccaccttcatgggtttacttgcgaaccacgcggggttgaacggactgttacaggctttgcgtgaaatggttgcctcgccacatgttgagttggcggttcatctttttgctgacttagccttagctacctctttccttggggttgcgctgggattattcgactatctggctgatctttttcaacgctccaacaccgtaggtggacgtttacagactggagccattactttcttgccccctttagcctttgcgctgttttatccacgtgggtttgttatggccttggggtatgctggagtcgccttagctgtacttgctcttattattccatcgttattaacgtggcaatcgcgtaaacacaacccccaagcagggtaccgcgtgaagggaggacgccccgcgctggtggttgtttttctgtgcgggattgccgtcatcggcgtgcaatttttgattgcagcaggtttgttgccggaggtgggg

SEQ ID NO: 88 - DNA, 1302 bp, Variovorax paradoxus
  misc_feature 1..1302: note = Beta-phenylalanine transaminase (Aromatic beta-amino acid aminotransferase;Beta-phenylalanineaminotransferase; VpAT)
  source 1..1302: mol_type = genomic DNA
atgactcatgctgcaattgaccaggcgttggcagacgcctatcgtcgttttactgacgcaaaccctgccagccagcgtcagtttgaagcgcaagcccgctatatgcccggggctaactctcgctctgttttgttttatgcaccctttccattgacgatcgcacgtggggaaggcgccgctctttgggatgcggacggccaccgttacgctgactttatcgcggaatacacagctggggtgtatggacacagtgccccagagattcgtgacgcagtaatcgaagctatgcagggtgggattaatttgacgggtcataatttgttggaaggccgcttagcccgccttatttgtgagcgtttcccacagatcgaacagttgcgtttcacgaatagcggaacagaggccaatctgatggcccttaccgcggcgcttcattttactggtcgccgcaaaatcgtcgtatttagtggaggttatcatgggggggttcttgggttcggtgcccgtcctagccctaccacagtaccatttgacttccttgtgctgccttacaacgatgctcagacggctcgtgctcagatcgagcgccacggcccggagatcgcggtcgtgttagtcgagcccatgcaaggtgcttctggctgcatcccaggtcagcccgactttctgcaagccctgcgcgaatccgctactcaggtaggggcgctgttagtttttgacgaagtgatgactagtcgcttagcgccacatggtttagctaacaaattggggatccgttcggatttgacaaccctgggtaagtacattggcggcggtatgtcatttggggcctttggcggtcgtgctgatgtcatggccctgttcgaccctcgcactggacctttggctcattccggtacgtttaacaacaatgtgatgacgatggctgccggttatgctggcttaacgaaattattcactccggaagcggcaggggcattggcagagcgtggagaagcgcttcgcgcacgtcttaacgccctgtgtgctaacgaaggagtagcaatgcagttcactggcatcggctcgctgatgaatgcccacttcgtccagggagacgttcgtagctctgaggatctggccgcagttgatgggcgtttacgtcagttgttgttctttcatttattgaatgaagatatttactcttcaccgcgtgggtttgttgtattatcgttgccattgactgacgctgatattgaccgctacgttgctgcgatcggttcatttattggcggtcatggggcgttgttaccgcgcgctaac

SEQ ID NO: 89 - DNA, 1401 bp, Escherichia coli
  misc_feature 1..1401: note = gadA glutamate decarboxylase (Escherichia coli)
  source 1..1401: mol_type = genomic DNA
atggaccagaagctgttaacggatttccgctcagaactactcgattcacgttttggcgcaaaggccatttctactatcgcggagtcaaaacgatttccgctgcacgaaatgcgcgatgatgtcgcatttcagattatcaatgatgaattatatcttgatggcaacgctcgtcagaacctggccactttctgccagacctgggacgacgaaaacgtccataaattgatggatttgtcgatcaataaaaactggatcgacaaagaagaatatccgcaatccgcagccatcgacctgcgttgcgtaaatatggttgccgatctgtggcatgcgcctgcgccgaaaaatggtcaggccgttggcaccaacaccattggttcttccgaggcctgtatgctcggcgggatggcgatgaaatggcgttggcgcaagcgtatggaagctgcaggcaaaccaacggataaaccaaacctggtgtgcggtccggtacaaatctgctggcataaattcgcccgctactgggatgtggagctgcgtgagatccctatgcgccccggtcagttgtttatggacccgaaacgcatgattgaagcctgtgacgaaaacaccatcggcgtggtgccgactttcggcgtgacctacaccggtaactatgagttcccacaaccgctgcacgatgcgctggataaattccaggccgacaccggtatcgacatcgacatgcacatcgacgctgccagcggtggcttcctggcaccgttcgtcgccccggatatcgtctgggacttccgcctgccgcgtgtgaaatcgatcagtgcttcaggccataaattcggtctggctccgctgggctgcggctgggttatctggcgtgacgaagaagcgctgccgcaggaactggtgttcaacgttgactacctgggtggtcaaattggtacttttgccatcaacttctcccgcccggcgggtcaggtaattgcacagtactatgaattcctgcgcctcggtcgtgaaggctataccaaagtacagaacgcctcttaccaggttgccgcttatctggcggatgaaatcgccaaactggggccgtatgagttcatctgtacgggtcgcccggacgaaggcatcccggcggtttgcttcaaactgaaagatggtgaagatccgggatacaccctgtacgacctctctgaacgtctgcgtctgcgcggctggcaggttccggccttcactctcggcggtgaagccaccgacatcgtggtgatgcgcattatgtgtcgtcgcggcttcgaaatggactttgctgaactgttgctggaagactacaaagcctccctgaaatatctcagcgatcacccgaaactgcagggtattgcccagcagaacagctttaaacacacctga

SEQ ID NO: 90 - DNA, 1398 bp, Escherichia coli
  misc_feature 1..1398: note = glutamate decarboxylase (Escherichia coliKO11FL)
  source 1..1398: mol_type = genomic DNA
atggataaaaagcaagtgacggacctgcgctctgaacttcttgacagtcgttttggggcaaagagtattagtaccattgctgagtcaaagcgttttcctttgcatgagatgcgcgatgacgtcgcattccagattatcaacgacgagctgtatttggacggcaatgcccgccaaaacttggccacgttttgtcagacttgggatgacgagaatgttcataaacttatggacctttcaattaacaaaaattggattgacaaagaagagtacccccaatctgccgcaattgatttacgttgtgttaatatggtggccgacttatggcatgcaccagcccctaaaaacggccaagcggtgggaaccaacacgatcgggtctagtgaggcatgtatgttaggcgggatggccatgaagtggcgttggcgtaaacgcatggaggcagcagggaaaccaaccgataaacctaatttagtctgcggaccggttcagatctgttggcataaatttgcgcgctactgggatgtggaattacgcgaaattccgatgcgtccgggccaactgttcatggatcccaaacgtatgatcgaagcatgtgacgaaaacacgattggggtggtacccacctttggggtcacatatacaggtaactacgagtttccacaaccgttgcatgatgctctggacaagtttcaagctgacaccgggatcgacattgatatgcacattgacgctgcctccggcggattcttggccccatttgtagcccctgacattgtctgggactttcgtcttccccgtgtgaaatccatcagcgcatccggtcacaagtttgggcttgccccattagggtgtggatgggtcatctggcgtgatgaggaagcattaccccaagaacttgtcttcaatgtagattaccttgggggacagattggcacttttgccatcaacttttctcgcccagcgggtcaagtgatcgcccagtattacgagtttctgcgcctgggacgtgagggatatacaaaagtgcagaacgcatcgtaccaggtagcggcttaccttgcggacgaaattgcaaagctgggaccatacgagtttatctgtaccgggcgtccagatgaaggtattccggctgtgtgttttaagctgaaagacggggaagatcccggatatacgctgtatgatctgtctgaacgtttacgtttgcgcggttggcaagttccagccttcacgttgggtggcgaagccactgatattgtagtcatgcgtatcatgtgtcgtcgcggctttgaaatggatttcgcagagttacttctggaagactacaaagcgagcttaaaatatttgtctgaccatcccaagttgcaagggatcgcacagcaaaattcgtttaaacacact

SEQ ID NO: 91 - DNA, 1287 bp, Bacillus atrophaeus
  misc_feature 1..1287: note = GltT (Bacillus atrophaeus UCMB-5137)
  source 1..1287: mol_type = genomic DNA
atgaagaaattacgcttcggactggcgactcaaatctttgtggggctgattcttggggtagtagtgggcgttatctggtacggtaatccggcggtggtaacttatttgcagccagttggggacctttttttacgtttgattaaaatgatcgttattcctatcgtggtgtcttctttgatcattggcgtcgcgggagctgggtccggaaaacaggtcggaaagctgggctttcgtactattctgtacttcgagatcatcactacctttgccatcattctgggacttgctctggcgaatcttttccagcctggtacaggagtaaatatcgagagcgcgcagaaaagtgacatttcccagtacgtggagactgaaaaagagcaatccaccaaatccgtagctgagactttcctgcatatcgtgcccaccaatttctttcaatcacttgcggaaggtgatcttcttgctattatctgctttaccgtacttttcgcccttggcatttcggctatcggtgaacgtggcaaaccggtgcttgctttctttgacggagtatcccacgcgatgtttcatgtagtgaaccttgtgatgaaggttgctccgttcggcgtatttgctctgattggagtaacagtaagcaaatttggactgggttctttactgagcctgggtaaacttgtggggctggtatatgttgctctggcattttttcttattgtaatctttggtattgttggaaagctggctggcgtgaatatcttcaagtttttagcttacatgaaggatgaaatcttattagcgttctcgacctcatcgtccgagactgtgttgccccgcatcatggagaaaatggagaagatcgggtgtccaaagggaattgtaagctttgtagtccccatcggttacacattcaatcttgacggctcggtcttataccaatctattgctgcgctgttcttggcacaggtttacggaatcgacctgactatttggcatcagattactctggtgttagttctgatggtcactagcaaaggcatggcagccgttcctggaactagctttgtagtcctgctggcaaccttaggtaccattggtgttccagcggaagggcttgcattcattgcgggggttgaccgcattatggacatggctcgcactgtggtcaatttaacaggcaatgctcttgcgagtgtcgtaatgagcaagtgggagggtcagtacgacccggtgaaaggtgcagagattatgagccgcagcaagacggaacaggacgctactatctccgga

SEQ ID NO: 92 - DNA, 858 bp, Escherichia coli
  misc_feature 1..858: note = mechanosensitive channelMscS(Escherichia coli)
  source 1..858: mol_type = genomic DNA
atggaggacttgaacgtagtagatagcattaatggagcgggctcatggttagtagccaaccaagccctgttgttatcgtatgctgtaaatatcgtcgcagccttagccatcattatcgttgggttaatcatcgcccgtatgatttctaatgcggtgaatcgcttaatgatctcgcgcaagatcgacgccactgtcgcggatttcttgtccgccctggtgcgttacggtatcatcgcgttcacattgattgcggcattagggcgcgtaggagtccagacagcttctgtgattgcggtattaggtgcagcaggattagctgtgggattggcgttacaggggtctctttccaatctggcggccggcgtacttctggttatgtttcgcccctttcgcgccggagagtatgtggatttgggaggagtggccggaacagtgctgtcagtgcaaatcttttctaccacgatgcgtacagcagatggaaaaatcatcgtgatccccaatggcaagatcatcgcgggtaacattatcaacttctcccgcgaacctgttcgccgcaacgaatttatcatcggtgttgcctatgattcagacatcgatcaggtcaaacaaattcttacgaacatcattcagtcagaggaccgtattctgaaagaccgcgaaatgacggtgcgtttgaatgagttaggggcttcaagtatcaacttcgtagtccgcgtgtggagcaattccggtgatttgcaaaacgtgtattgggacgtccttgagcgcattaagcgtgaattcgatgctgccgggatctcctttccgtatcctcagatggatgtgaatttcaagcgtgtaaaggaagataaggctgcc

SEQ ID NO: 93 - DNA, 1539 bp, Bacillus amyloliquefaciens
  misc_feature 1..1539: note = HutH (Bacillus amyloliquefaciens subsp. plantarum str. FZB42)
  source 1..1539: mol_type = genomic DNA
atgatggtcaccttggatgggtcttcattaacgacggctgatgcacaacgtgtacttttcgattttgaagaggtacaggcatcggctgaatcgatggagcgcgtaaaaaagagccgtgccgccgtggaacgcattgtacaagaagaaaaaactatctacggaatcactacggggtttggtaagttttccgatgtgctgatccaaaaagaggacgctgcggatttacaattgaatttgatcttgtcacatgcatgtggagtcggcgatcctttcccagagtcagtctcccgcgccatgctgcttctgcgtgcaaacgcattgttaaaaggcttctccggtgttcgtacggaattaattgaccagcttttagcgtacttaaaccaccgtatccaccctgttatcccccaacaaggttcgctgggggcctccggcgatttggcccctcttagccaccttgcgttggcactgatcggacaaggggaagtgttctacgaaggagcacgtatgcccactgctcatgcccttgaacaaaccaatctgcagcccgcagtcctgacatcgaaggaagggctggcgttgatcaatgggactcaggctatgaccgcaatgggcttaatcgcataccttgaagccgaaaagttggcatatcagagcgagcgcatcgcttcattgactatcgaaggattgcaaggtattattgacgcgtttgacgaagatattcatgccgctcgtggataccaggaacaaatggatgtcgctgagcgcattcgctattatctttcggattcgaagctgacaaccgtacaaggcgagctgcgtgtgcaagatgcttactccattcgctgcatccctcaagtccacggagcttcttggcagaccctggcgtatgtgaaggagaagttagaaattgagatgaacgctgctactgataaccctttaatttttgaagacggggccaaaattatctcgggggggaactttcacgggcaaccgatcgcgtttgcaatggacttcttgaaagtagctgctgctgagttggctaatatcagcgagcgccgtattgagcgtcttgtcaatccacagctgaatgaccttcctccttttctttcgccgcaaccgggtttacagtctggtgccatgattatgcagtacgccgctgcctccttggtctcggaaaacaaaacacttgcgcatcccgcctcagtcgactcaatcccctcctcggctaaccaggaggatcacgtctccatggggacgatcgcttcacgtcatgcttaccagattattgcaaacactcgtcgcgtattagccgtcgaggccatttgcgctttacaagctgtagagtaccgtggggaagagcactgcgctagctacacgaaacaactttaccatgagatgcgtaacatcgtgccatcgattcaggaggaccgtgttttctcgtacgacatcgagcacttatccgactggcttaaaaaggaatccttcttacctaatgaacaccaccaaaagttaatgactaatgagggcgggttaactcgc

SEQ ID NO: 94 - DNA, 780 bp, Escherichia coli
  misc_feature 1..780: note = Histidine ABC transporter, histidine-binding periplasmic protein precursorHisJ (Escherichia coliO145:H28 str. RM12581])
  source 1..780: mol_type = genomic DNA
atgaagaaacttgtcctttcattgtctctggtattagcgttcagttcagcaactgcagcattcgctgctattccgcaaaatatccgcatcgggacggatcccacgtatgcgccattcgagtcaaagaattcacaaggtgaattggtcgggttcgatattgacctggcgaaagaattgtgtaaacgtatcaatacccaatgcacgttcgtggaaaatcccttggatgcattaattccgtctttgaaagcgaaaaaaatcgatgccatcatgtcatccctttctatcacagaaaagcgccagcaggagattgccttcacagacaagttgtacgctgcagacagccgcctggtcgttgcaaagaattctgacattcaacctaccgtggaatcgctgaagggcaagcgcgtaggggtcttgcagggcactactcaggaaacatttgggaacgaacattgggcgcctaagggaattgagatcgtgtcttatcagggtcaggataacatctacagtgatctgacagccggacgtattgacgccgcttttcaggacgaggtggcggcatctgaagggttcttaaagcagccagtcggcaaagactacaaatttggtgggccgagcgtgaaggacgagaaattgtttggggtaggaacagggatgggcttgcgtaaggaggacaatgaattacgtgaagctcttaataaagcctttgctgagatgcgtgcggacgggacttacgaaaaacttgcaaaaaagtatttcgactttgacgtctacggcggt

SEQ ID NO: 95 - DNA, 684 bp, Escherichia coli
  misc_feature 1..684: note = Histidine ABC transporter, permease proteinHisQ (Escherichia coliO145:H28 str. RM12581)
  source 1..684: mol_type = genomic DNA
atgctgtatggattcagtggcgttatcttgcagggggctcttgtcactttagagttagctatctcgtccgttgtgttagctgtcattattggacttatcggggctggtggcaaattgagtcagaaccgtttgagcggccttatttttgaagggtacacaaccttaattcgcggagtcccagacttagtgctgatgttgcttattttctatggtttacagatcgctttgaatacggttaccgaggcaatgggggtcggccaaatcgatatcgatcctatggtggctggaatcattactttgggcttcatttacggggcatatttcacggagacgttccgcggagctttcatggccgtcccgaagggccacattgaagcggcaacagcttttggattcactcgtgggcaagttttccgtcgcatcatgtttccagcgatgatgcgctatgcgcttcctgggatcgggaataactggcaggtaatcttaaaatcgacggctttagtcagtttattggggttggaagatgtcgtaaaagcgacccagttggctgggaaatcgacttgggagcccttttacttcgctattgtgtgtggcgttatttacttagttttcactacagtatcaaacggtgtgttattgtttttggaacgtcgctacagcgtgggtgtaaagcgtgctgatttg

SEQ ID NO: 96 - DNA, 771 bp, Escherichia coli
  misc_feature 1..771: note = hisP (Escherichia coliEPEC C342-62)
  source 1..771: mol_type = genomic DNA
atgtccgagaacaaattaaatgttatcgatttgcataagcgttatggagagcatgaagtgttgaaaggagtgtctcttcaagcaaacgcgggggacgtaatttctatcatcggatcgtctggttctggtaagtcaaccttcctgcgttgtattaacttcttagagaagccgtctgagggttctattgtagttaatgggcagaccatcaatcttgtgcgcgataaggacggccagttgaaagtggcagacaaaaaccaacttcgtttgcttcgcacccgtcttaccatggtattccaacacttcaacctgtggtcgcacatgacggtacttgagaacgtgatggaagcgccaattcaggtacttggattgagcaaacaagaagcccgcgaacgtgcggtgaaatatttggccaaggtgggtatcgacgagcgtgcgcagggcaaataccccgttcacttgtccgggggtcaacaacagcgtgtcagtattgcccgcgctctggctatggaaccagaggtgcttctgtttgacgagccgacgtcagctttggacccggaattagtgggcgaagtattgcgcatcatgcagcagttagcagaagaaggcaagaccatggttgttgtcacacacgaaatggggtttgcgcgtcatgtctcgactcatgtaatcttcttgcatcaaggtaaaatcgaggaagaaggagcgccggaacagttattcgggaatcctcaatccccccgtctgcagcagtttcttaaagggtccttaaag

SEQ ID NO: 97 - DNA, 660 bp, Clostridium botulinum
  misc_feature 1..660: note = proline reductase (Clostridium botulinum)
  source 1..660: mol_type = genomic DNA
atgtcaatgtccgctgagcacgctgaggaattaaaaaatgaacctgcggtcgtttgttgtcgcactgaggaggggaccatcttgtcagccgataatttggaagacccaaacatttttccagatatggtggatagcggtttactgaacattcctggggactgcttaaaagttggggaagtaatcggggccaaactgcttaagacgattgactctttgacccctcttgccaaggacatcattgagggggccaaatccttagacggagacgtacgcagtaaatcagagattcagatcgaatcaccagaggagaaggcgatccttaaaaacaatttgaaggcgggagatattatcaaggttgaggacctggagaaccctatgcacttcgccaagttacaagattcgcttcttatcaagctggatgagaaagtgcttacgcgccgcgaagttgtagacgcgaaacttacggaagatgcaccggcgatttcaggggtcactgcatcaatgttggaaggcttcgaggaaaaggccctggagattacccaagatagcaaggatgtggacttcaattcagtaattccactgaacggcaatcgtgaattccttcgtttgaaaatcgaggaaggcacaggcatttatatcgaaattccctttacccaagtc

SEQ ID NO: 98 - DNA, 1347 bp, Escherichia coli
  misc_feature 1..1347: note = ProlineporterII (Escherichia coli PMV-1)
  source 1..1347: mol_type = genomic DNA
atgtcagaaaaacttccggcacctcgcgagggtttatccggtaaagctatgcgtcgtgttgtcatgggtagctttgccggtgcattaatggaatggtatgatttcttcatctttgggacggcggcgggtcttgtttttgcaccgctgttttatcctgacagtgatccgtttattgggttgatcgcgtcgttcgctacatttggagttggttttttgacccgcccgttaggaggtatcgtgttcggtcattttggtgacaagatcgggcgtaagattaccttaatctggacattggcgattgtggggtgttctacattcttaatcggtttcattccaacgtaccaagaaatcggcatttgggcccctttggtccttatggttttgcgcctgattcagggttttggcttgggaggagaatacggaggggcggcgttaatgaccatcgaaagtgcccccgaaagccgccgtggttttcttgggtcattgccacagacggccgccagcgtcggcatcatgcttgcaacgggtattttcgcgctttgtaatcatttccttacttctgaacagttcttatcatggggctggcgtattcccttctggttgtccgcggttatgttaatcgtcggactttttatccgtctgcatactgaagagacgcttgactttcagaagcaaaaaacgactaacaataaagaaaagtcggttcccccccttatcgagctttttaagaaacatccacgcaatattttgttggccttgggggcccgccttgcggagtcagtaagcagcaacatcattaatgcattcggcatcgtctatatcagcagtcaacttgcactgagccgtgacatccccttgactgggatgctgattgcaagcgccatcggaatttttagttgcccattggtaggctggctttcggaccgtatcggacagaaatcattatatttgtcaggcgctggattttgtgtcctgtttgccttcccgttttttctgctgttggactcgaagagtacactgattatctggtgctcaatgattttgggctataacttgggtccaactatgatgtttgctgtacaaccaacattgtttactcgtatgttcggcaccaaggtccgctacacaggcttatcatttgcttaccagttctcggctatcttaggcggcctgtccccactgattgcatcctcacttcttgcgttggggggcggcaaaccctggtatgtcgccttgttccttttcgctgtgtccgtgttatctttcgtctgtgtatggttaatcgagcccacagacgaacaagagacggcttcataccgctacatccgcgaacaaagtcatgagaac

SEQ ID NO: 99 - DNA, 1377 bp, Escherichia coli
  misc_feature 1..1377: note = Escherichia coli PheP
  source 1..1377: mol_type = genomic DNA
atgaaaaacgcgtcaaccgtatcggaagatactgcgtcgaatcaagagccgacgcttcatcgcggattacataaccgtcatattcaactgattgcgttgggtggcgcaattggtactggtctgtttcttggcattggcccggcgattcagatggcgggtccggctgtattgctgggctacggcgtcgccgggatcatcgctttcctgattatgcgccagcttggcgaaatggtggttgaggagccggtatccggttcatttgcccactttgcctataaatactggggaccgtttgcgggcttcctctctggctggaactactgggtaatgttcgtgctggtgggaatggcagagctgaccgctgcgggcatctatatgcagtactggttcccggatgttccaacgtggatttgggctgccgccttctttattatcatcaacgccgttaacctggtgaacgtgcgcttatatggcgaaaccgagttctggtttgcgttgattaaagtgctggcaatcatcggtatgatcggctttggcctgtggctgctgttttctggtcacggcggcgagaaagccagtatcgacaacctctggcgctacggtggtttcttcgccaccggctggaatgggctgattttgtcgctggcggtaattatgttctccttcggcggtctggagctgattgggattactgccgctgaagcgcgcgatccggaaaaaagcattccaaaagcggtaaatcaggtggtgtatcgcatcctgctgttttacatcggttcactggtggttttactggcgctctatccgtgggtggaagtgaaatccaacagtagcccgtttgtgatgattttccataatctcgacagcaacgtggtagcttctgcgctgaacttcgtcattctggtagcatcgctgtcagtgtataacagcggggtttactctaacagccgcatgctgtttggcctttctgtgcagggtaatgcgccgaagtttttgactcgcgtcagccgtcgcggtgtgccgattaactcgctgatgctttccggagcgatcacttcgctggtggtgttaatcaactatctgctgccgcaaaaagcgtttggtctgctgatggcgctggtggtagcaacgctgctgttgaactggattatgatctgtctggcgcatctgcgttttcgtgcagcgatgcgacgtcaggggcgtgaaacacagtttaaggcgctgctctatccgttcggcaactatctctgcattgccttcctcggcatgattttgctgctgatgtgcacgatggatgatatgcgcttgtcagcgatcctgctgccggtgtggattgtattcctgtttatggcatttaaaacgctgcgtcggaaataa

SEQ ID NO: 100 - DNA, 1704 bp, Anabaena variabilis
  misc_feature 1..1704: note = Anabaena variabilis PAL1
  source 1..1704: mol_type = genomic DNA
atgaaaacactatcacaggcccaatctaaaacttcttcacagcaattcagctttaccgggaactcgtctgcgaatgtaattatcggcaatcaaaagctgaccattaatgatgtagctcgcgttgcccggaatggcactttggtgtcactgacgaacaataccgacattctgcaaggtattcaagctagctgcgattatatcaataacgccgttgaatctggcgagccaatctacggggtaacaagcggttttggtgggatggcgaacgttgccattagccgtgaacaggcgagcgaacttcagaccaacctcgtttggttcctaaagacaggagctggtaataagttacctctggctgacgtaagagccgcgatgctgcttcgcgctaatagtcacatgcgcggcgccagtggtatccgtcttgagcttatcaagaggatggaaatcttcctcaacgcgggtgtcacaccatatgtttatgagtttggtagtatcggagccagtggtgatcttgttcccctgagttatattacgggttcattgattggtttagacccgtcctttaaagtggattttaacgggaaagaaatggacgccccgaccgctttacgacagcttaatctgagcccacttactttgctccctaaagaaggtcttgccatgatgaatggcacctctgtgatgactggaattgccgcgaattgtgtgtatgacacgcagatcctaacggccattgccatgggtgttcacgcgttggacattcaagccctgaatggtacaaaccagtcgtttcatccgtttatccataattcaaaaccccatccgggacagctttgggctgctgatcagatgatctcactcctggccaatagtcaactggttcgggacgagctcgacggcaaacatgattatcgcgatcatgagctcatccaggaccggtattcacttcgttgtctcccacaatacctggggcctatcgttgatggtatatctcaaattgcgaagcaaattgaaattgagatcaatagcgtaaccgacaacccgcttatcgatgttgataatcaggcctcttatcacggtggcaattttctgggccagtatgttggtatggggatggatcacctgcggtactatattgggcttctggctaaacatcttgatgtgcagattgccttattagcttcaccagaattttcaaatggactgccgccatcattgctcggtaacagagaaaggaaagtaaatatgggccttaagggccttcagatatgtggtaactcaatcatgcccctcctgaccttttatgggaactcaattgctgatcgttttccgacacatgctgaacagtttaaccaaaacattaactcacagggctatacatccgcgacgttagcgcgtcggtccgtggatatcttccagaattatgttgctatcgctctgatgttcggcgtacaggccgttgatttgcgcacttataaaaaaaccggtcactacgatgctcgggcttgcctgtcgcctgccaccgagcggctttatagcgccgtacgtcatgttgtgggtcagaaaccgacgtcggaccgcccctatatttggaatgataatgaacaagggctggatgaacacatcgcccggatatctgccgatattgccgccggaggtgtcatcgtccaggcggtacaagacatacttccttgcctgcattaa

SEQ ID NO: 101 - DNA, 1599 bp, Photorhabdus luminescens
  misc_feature 1..1599: note = Photorhabdus luminescens PAL3
  source 1..1599: mol_type = genomic DNA
atgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttctttctgctggtactggggactatatgtccaaaccttgtattaaagcgtcacaatttactatgttactttctgtttgcaaaggttggtctgcaaccagaccaattgtcgctcaagcaattgttgatcatattaatcatgacattgttcctctggttcctcgctatggctcagtgggtgcaagcggtgatttaattcctttatcttatattgcacgagcattatgtggtatcggcaaagtttattatatgggcgcagaaattgacgctgctgaagcaattaaacgtgcagggttgacaccattatcgttaaaagccaaagaaggtcttgctctgattaacggcacccgggtaatgtcaggaatcagtgcaatcaccgtcattaaactggaaaaactatttaaagcctcaatttctgcgattgcccttgctgttgaagcattacttgcatctcatgaacattatgatgcccggattcaacaagtaaaaaatcatcctggtcaaaacgcggtggcaagtgcattgcgtaatttattggcaggttcaacgcaggttaatctattatctggggttaaagaacaagccaataaagcttgtcgtcatcaagaaattacccaactaaatgataccttacaggaagtttattcaattcgctgtgcaccacaagtattaggtatagtgccagaatctttagctaccgctcggaaaatattggaacgggaagttatctcagctaatgataatccattgatagatccagaaaatggcgatgttctacacggtggaaattttatggggcaatatgtcgcccgaacaatggatgcattaaaactggatattgctttaattgccaatcatcttcacgccattgtggctcttatgatggataaccgtttctctcgtggattacctaattcactgagtccgacacccggcatgtatcaaggttttaaaggcgtccaactttctcaaaccgctttagttgctgcaattcgccatgattgtgctgcatcaggtattcataccctcgccacagaacaatacaatcaagatattgtcagtttaggtctgcatgccgctcaagatgttttagagatggagcagaaattacgcaatattgtttcaatgacaattctggtagtttgtcaggccattcatcttcgcggcaatattagtgaaattgcgcctgaaactgctaaattttaccatgcagtacgcgaaatcagttctcctttgatcactgatcgtgcgttggatgaagatataatccgcattgcggatgcaattattaatgatcaacttcctctgccagaaatcatgctggaagaataa

SEQ ID NO: 102 - DNA, 819 bp, Legionella pneumophila
  misc_feature 1..819: note = Legionella pneumophila phhA
  source 1..819: mol_type = genomic DNA
atggagtttagtagccggtatgtcgcacatgtccctgatgctcagggtttagtcgattattcggcacaagaaaatagaatttggaatattttatttgagaggcaactcaagttattgccaggaagagcttgtgatgaatttctgtctggattacagactttaggacttaactcctcgactattccacaacttccagaagtaagtgagcgattaaaggccaaaacgggatggcaagtagcgccagttgctgctttaatttcagccagggaattttttgaattattagcagaaaaatattttcctgcggcgacttttattcgaagtgaagaagaattggattatgttcaagaacctgatatttttcatgagctttttggtcattgtcctatgttaaccgatagagtctatgctgaatttgtccatgattacgcatgtaaggtattaacttttcctgaacaggattggcctttattgcaaagaatgttttggtttactgtagagtttggattgattaaaacgcctaaagggcttagagcatacggcgggggaattttatcttctatcagtgaaacggtatattgtgtggaaagtgatattcctgtgcgaattttatttgatccagtggtggcttttcgaatgccttatcggattgaccagctacaacctgtttatttcgttattgacagctatcaaaatttatatgatttcgtgctttctgacatgggtaaattcatggatcgtgcgcgagagttaggtgaatttccaccgtattttgatgtggatccggataatccaaatattcatataagggcttgttaa

SEQ ID NO: 103 - DNA, 717 bp, Escherichia coli
  misc_feature 1..717: note = Escherichia coli hisM
  source 1..717: mol_type = genomic DNA
gtgatcgaaatcttacatgaatactggaaaccgctgctgtggaccgacggttatcgctttactggtgtggcgatcactctgtggctgcttattttgtcggtagtgataggcggagtcctggcgctgtttctggcgattggtcgtgtctccagtaataaatacatccagtttccaatctggttatttacctatatttttcgcggtacgccgctgtatgttcagttgctggtgttctattccggcatgtacacgcttgagattgttaagggaaccgaattccttaacgctttcttccgcagtggcctgaactgtaccgtgctggcgctgacgcttaacacctgcgcttacactaccgagatttttgctggggcaatccgttcggttccgcatggggaaattgaagccgccagagcctatggcttctcgacttttaaaatgtatcgctgcattattttgccttctgcgctgcgtattgcgttaccggcatacagcaacgaagtgatcctgatgctgcactctactgcgttggcatttactgccacggtgccggatctgctgaaaatagcccgcgatattaacgccgccacgtatcaaccttttaccgccttcggcattgccgcggtgctctatttaatcatctcttatgtcctgatcagcctctttcgcagagcggaaaaacgctggttgcagcatgtgaaaccttcttcaacgcactga

SEQ ID NO: 104 - DNA, 967 bp, unidentified
  misc_feature 1..967: note = clbA (wild-type)
  source 1..967: mol_type = genomic DNA
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 105 - DNA, 424 bp, unidentified
  misc_feature 1..424: note = clbA knock-out
  source 1..424: mol_type = genomic DNA
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 106 - DNA, 1921 bp, synthetic construct
  misc_feature 1..1921: note = Synthetic: Prp promoter
  source 1..1921: mol_type = other DNA
ttacccgtctggattttcagtacgcgcttttaaacgacgccacagcgtggtacggctgatccccaaataacgtgcggcggcgcgcttatcgccattaaagcgtgcgagcacctcctgcaatggaagcgcttctgctgacgagggcgtgatttctgctgtggtccccaccagttcaggtaataattgccgcataaattgtctgtccagtgttggtgcgggatcgacgcttaaaaaaagcgccaggcgttccatcatattccgcagttcgcgaatattaccgggccaatgatagttcagtagaagcggctgacactgcgtcagcccatgacgcaccgattcggtaaaagggatctccatcgcggccagcgattgttttaaaaagttttccgccagaggcagaatatcaggctgtcgctcgcgcaaggggggaagcggcagacgcagaatgctcaaacggtaaaacagatcggtacgaaaacgtccttgcgttatctcccgatccagatcgcaatgcgtggcgctgatcacccggacatctaccgggatcggctgatgcccgccaacgcgggtgacggctttttcctccagtacgcgtagaaggcgggtttgtaacggcagcggcatttcgccaatttcgtcaagaaacagcgtgccgccgtgggcgacctcaaacagccccgcacgtccacctcgtcttgagccggtaaacgctccctcctcatagccaaacagttcagcctccagcaacgactcggtaatcgcgccgcaattaacggcgacaaagggcggagaaggcttgttctgacggtggggctgacggttaaacaacgcctgatgaatcgcttgcgccgccagctctttcccggtccctgtttccccctgaatcagcactgccgcgcgggaacgggcatagagtgtaatcgtatggcgaacctgctccatttgtggtgaatcgccgaggatatcgctcagcgcataacgggtctgtaatcccttgctggaggtatgctggctatactgacgccgtgtcaggcgggtcatatccagcgcatcatggaaagcctgacgtacggtggccgctgaataaataaagatggcggtcattcctgcctcttccgccaggtcggtaattagtcctgccccaattacagcctcaatgccgttagctttgagctcgttaatttgcccgcgagcatcctcttcagtgatatagcttcgctgttcaagacggaggtgaaacgttttctgaaaggcgaccagagccggaatggtctcctgataggtcacgattcccattgaggaagtcagctttcccgcttttgccagagcctgtaatacatcgaatccgctgggtttgatgaggatgacaggtaccgacagtcggctttttaaataagcgccgttggaacctgccgcgataatcgcgtcgcagcgttcggttgccagttttttgcgaatgtaggctactgccttttcaaaaccgagctgaataggcgtgatcgtcgccagatgatcaaactccaggctgatatcccgaaatagttcgaacaggcgcgttaccgagaccgtccagatcaccggtttatcgctattatcgcgcgaagcgctatgcacagtaaccatcgtcgtagattcatgtttaaggaacgaattcttgttttatagatgtttcgttaatgttgcaatgaaacacaggcctccgtttcatgaaacgttagctgactcgtttttcttgtgactcgtctgtcagtattaaaaaagatttttcatttaactgattgtttttaaattgaattttatttaatggtttctcggtttttgggtctggcatatcccttgctttaatgagtgcatcttaattaacaattcaataacaagagggctgaatagtaatttcaacaaaataacgagcattcgaatg

SEQ ID NO: 107 - DNA, 864 bp, Salmonella enterica
  misc_feature 1..864: note = Tsx - Salmonella enterica subsp. enterica serovar Typhimurium LT2 (STM0413)
  source 1..864: mol_type = genomic DNA
atgaaaaaaactttactcgcagtcagcgcagcgctggcgctcacctcatcttttactgctaacgcagcagaaaatgatcagccgcagtatttgtccgactggtggcaccagagcgtaaacgtggtaggcagctaccatacccgtttctcgccgaaattgaacaacgacgtctatctggaatatgaagcatttgccaaaaaagactggtttgatttctacggctatatcgatattcccaaaacctttgattggggtaacggcaacgataaaggtatctggtccgacggttctccgctgttcatggaaatcgaaccgcgtttctcaattgataagctgaccggcgcagacctgagcttcggcccgtttaaagagtggtatttcgccaacaactacatctacgatatgggcgataacaaagccagccgccagagcacgtggtatatgggtctggggaccgatatcgacaccggcctgccgatgggtctgtcgctgaacgtgtatgcgaaatatcagtggcaaaactacggcgcgtccaatgaaaacgaatgggacggctaccgtttcaaagtgaaatacttcgtccccatcaccgatctgtggggcggtaaactgagctatatcggctttaccaactttgactggggatctgatttaggcgacgatccgaaccgtaccagcaactccatcgcttccagccatatcctggcgctgaactacgatcactggcactactcggtcgttgcgcgttacttccataacggcggacagtggcagaatggcgcaaaactgaactggggcgacggcgatttcagcgcgaaatctaccggctggggcggctacctggtcgtgggttacaacttctaa

SEQ ID NO: 108 - DNA, 885 bp, Escherichia coli
  misc_feature 1..885: note = Tsx - Escherichia coli K-12 MG1655 (b0411)
  source 1..885: mol_type = genomic DNA
atgaaaaaaacattactggcagccggtgcggtactggcgctctcttcgtcttttactgtcaacgcagctgaaaacgacaaaccgcagtatctttccgactggtggcaccagagcgttaacgttgtcggaagctatcacacccgtttcggaccgcagatccgcaacgatacctaccttgagtacgaagcattcgctaaaaaagactggttcgacttctatggttatgcggatgcgccggtattcttcggcggtaactccgatgctaaaggtatctggaaccacggttctccgctgtttatggaaatcgaaccacgtttctccatcgacaagctgaccaatactgaccttagcttcggtccgttcaaagagtggtacttcgcgaacaactacatttacgacatgggtcgtaataaagatggtcgccagagcacctggtacatgggtctgggtaccgatatcgacactggcctgccgatgagcctgtccatgaacgtctatgcgaaataccagtggcagaactatggcgcagcgaacgaaaacgagtgggacggttaccgtttcaaaattaaatactttgtgccgattaccgatctgtggggcggtcagctgagctacatcggcttcaccaacttcgactggggttccgatttaggggatgacagcggtaacgcaatcaacggtattaagacccgtactaataactctatcgcttccagccatattctggctctgaactacgatcactggcactactctgtcgtagctcgttactggcacgacggtggtcagtggaacgacgatgcagaactgaacttcggcaacggcaacttcaacgttcgctctaccggctggggtggttacctggtagtaggttacaacttctga

SEQ ID NO: 109 - DNA, 1221 bp, Bacillus halodurans
  misc_feature 1..1221: note = BH1446 - Bacillus halodurans (BAB05165)
  source 1..1221: mol_type = genomic DNA
atgaatattttgtggggtttattaggaatcgtcgttgtttttctaatcgcttttgcattttccacaaatcgtcgtgcaattaaaccacgaacgatattaggtggtctcgcgattcagctattatttgcgattattgtattaaaaattccagctggacaagcgttacttgagagcttaaccaatgtagttttgaacattattagttatgcgaatgaagggatcgacttcgtatttggtggatttttcgaagaaggttcaggcgtaggcttcgtttttgcaattaacgttttgtctgtcgtcattttcttctcagcactaatctcgatcctttattatttagggatcatgcaatttgtcattaaaattatcggtggtgcgctgtcctggctactcggaacatcaaaggcagaatcaatgtcagcagcagctaacattttcgttgggcaaacggaagcgccactcgttgttaagccatacttaccaaaaatgacgcaatccgagctctttgcggttatgaccgggggacttgcttctgttgctggttctgttttaatcggttattctcttttaggagtaccgctacaatatttattagcggcaagctttatggctgctcctgcgggcttgattatggcgaaaatgatcatgcctgaaacggagaaaacaaccgatgcagaagatgactttaagctcgcaaaggatgaagagtccacgaacttgattgacgcggccgccaatggggcgagcactgggttaatgctcgttctaaatattgcggcgatgttactagcgttcgttgcattgattgcattaattaatggaattcttggatggatcggaggattgtttggggcgtcgcaattgtctttagagttaatcctcggatacgtgtttgctccgcttgcgtttgtcatcggaattccttgggctgaagcgcttcaagcgggaagctacatcggacagaaactcgtagtgaacgaatttgttgcctacttaagctttgcaccagaaattgaaaacctttcagataaagcggtgatggtgattagttttgccctttgcggatttgctaacttctcatccctcggaatccttttaggaggattgggtaagcttgctccgagccgtcgccctgatattgcccgtctcggattacgcgcgatccttgcaggtacgctagcttctttactcagcgcctccattgcgggaatgttattctaa

SEQ ID NO: 110 - DNA, 1182 bp, Bacillus subtilis
  misc_feature 1..1182: note = nupC Bacillus subtilis subsp. subtilis 168 (BSU39410; CAA57663)
  source 1..1182: mol_type = genomic DNA
atgaagtatttgattgggattatcggtttaatcgtgtttttaggcctcgcgtggatcgcgagcagcggcaaaaaaagaattaagatccgcccaattgttgttatgctcattttgcaatttattcttggctacattctcctcaataccggaatagggaatttcctcgtgggaggatttgcaaaaggattcggttacctgcttgaatacgcggcagagggaattaactttgtgtttggcggcttggtgaatgcggaccaaacgacattctttatgaatgttctcttgccaatcgtgtttatttccgctctgatcgggattctgcaaaagtggaaagtcctcccgtttatcattagatatatcggccttgccctcagcaaggtaaacggtatgggaagattggaatcgtataacgcagtggcttctgcgattttagggcagtcagaagtatttatctccttgaagaaagaactcggtcttttaaatcagcagcgcttgtacacgctttgcgcatctgcgatgtcaactgtatcaatgtcgattgtcggtgcgtatatgacaatgctgaaaccggaatatgttgtaacagcgcttgttttgaacttatttggcggtttcattatcgcttctattatcaatccgtacgaggttgcaaaagaagaggatatgcttcgtgttgaggaagaagaaaaacaatccttcttcgaagtgctcggagaatacattcttgacggtttcaaagtagcggttgtcgtcgctgcgatgctgattggatttgtcgcgattattgcattgatcaatggcatttttaatgcagtattcggtatttcgttccaaggcattcttggatatgtgtttgctccattcgcttttcttgtcggtatcccatggaatgaagctgttaatgcgggaagcattatggcaacaaaaatggtatcgaatgaatttgtcgccatgacgtcgcttacgcaaaacggtttccatttcagcggccgtacaacagcgatcgtatcggtattccttgtgtcatttgcgaacttctcctcaatcggaatcattgccggtgccgtaaaaggactgaatgaaaagcaaggaaatgtcgtcgctcgtttcggcttgaaattattatacggtgctacgcttgtcagctttttatcagcagcaattgtgggcttgatttactga

SEQ ID NO: 111 - DNA, 1215 bp, Bacillus subtilis
  misc_feature 1..1215: note = yutK - Bacillus subtilis subsp. subtilis 168: BSU32180
  source 1..1215: mol_type = genomic DNA
atgaatgttctgtgggggctgctgggcgcagttgcgatcattgctatcgcgtttttattttcagaaaagaaaagcaatattaagataagaaccgtcatcgttggtttatgcacacaggtggcgtttggatacatcgtgttgaaatgggaagcgggacgcgctgtttttttatggttttcaagccgtgtacagcttctgattgactatgcgaatgaaggcatcagttttatttttggaccgcttctaaaggtcggagacagtccggcatttgcattaagtgtactgcccgttatcattttcttctcagcactgattgcagttttatatcatttgaaaatcatgcagctcgttttccgtgtcattggcggcggattgtcgaagctccttggaacaagcaaaacggaatctctggcggctgctgccaatatttttgtaggacaatcagaatctccgttagtgatcaaacccctgattgccgggctgacgcgctctgagttgtttacgattatgacgagcggtctatcggcagttgcgggatctaccttgtttgggtacgcgcttctcggtattccgattgagtacttgctggcggccagctttatggctgctccagctggactagtctttggtaaattgattatacccgaaacggaaaaaacgcaaaccgtaaaaagcgatttcaaaatggatgaaggcgaaggcgcagccaatgtcattgacgcagctgcaaagggagcgtcaacaggactgcaaattgcgttaaatgttggggcgatgctgcttgcgtttgttgcgttaatcgctgtagtaaacggtattctcggcggggctttcggcttgttcggtttaaaaggcgtaacattagaatccattctcggctatgtgttttctcctatcgcctttttgattggcgtgccttggcatgaagcattgcaggcgggaagctatatcggccagaaattggtgctgaatgagtttgtcgcttattctaacttcggttcgcacatcggcgagttttctaagaaaactgctaccattatcagtttcgcgttatgcggattcgccaatttttcatcaattgcgattatgcttggtacgcttggcggtttagcgcccagccgccgttcagatatcgcacgtctcggcctgaaggctgttcttgcaggaacattagccaatctgctcagcgcagccattgccggcatgtttatataa

SEQ ID NO: 112 - DNA, 1194 bp, Bacillus subtilis
  misc_feature 1..1194: note = yxjA - Bacillus subtilis subsp. spizizenii W23 (BSUW23_19355)
  source 1..1194: mol_type = genomic DNA
atgtactttttattaaaccttgtcggtctcattgtgattatggcagttgtgttcctatgctccccgcagaaaaagaaaatccagtggcgtccgatcattacgttaattgttctggaattgctgattacttggtttatgctgggaacaaaggtcgggagctgggccatcggtaaaattggtgatttcttcacttggctgattgcttgcgccagtgacggtatcgcgtttgccttcccgtcagtcatggcgaatgaaacagtagactttttctttagtgcacttcttccaattatctttatcgtcacattctttgatattttaacatatttcggcattttgccttggctgattgataaaatcggatgggtgatttcaaaggcttcccgcttgccgaaattagaaagctttttctctattcaaatgatgttcttgggaaatactgaagcacttgcggtcatccgccagcagcttacggtattaaataacaaccgcttgcttacatttggcttaatgagcatgagcagcatcagcggctccattattggatcttacctgtcaatggtgccggcgacatacgtgtttacagcgattccattgaactgcttaaacgcgctgattattgcaaacctgctgaaccctgttcatgtgccggaggatgaagatatcatctatacaccgcctaaagaagagaagaaagactttttctctacgatttctaacagtatgcttgtcggcatgaacatggttatcgttattttggcaatggtgatcggatatgtagcattaacgtctgcagtcaatggcattcttggtgttttcgtacacggcctgaccatccagacaatttttgcttatctcttcagtccgttcgcattcctgcttggtctgccagtacatgatgcaatgtatgtcgctcagctaatgggaatgaaattggcaacgaacgagtttgttgcgatgcttgacttgaaaaacaatcttacaacacttccgcctcacacagttgcggtggcgacgacattcctgacgtcatttgccaacttcagtactgtcggcatgatttacggaacgtacaactcgatccttgacggcgaaaagtcaacggtcatcgggaaaaacgtgtggaaattgctcgtcagcggcattgcggtatctttactaagtgctgcgattgtcggcctgtttgtgtggtag

SEQ ID NO: 113 - DNA, 1281 bp, Caulobacter crescentus
  misc_feature 1..1281: note = ccCNT (CC2089) - Caulobacter crescentus CB15 (AAK24060)
  source 1..1281: mol_type = genomic DNA
atgttccgtcccgagaacgttcaggccctcgcgggtctggcgctcaccctgggcctgtgctggctcgtttccgagaatcgcaagcggttcccctggggcctggccatcggcgcggtcgtcattcaggtcctgctggtcctggtcctgttcggcctgccgcaagcccagcagatgctgcgcggcgtcaacggcgcggtggagggccttgccgcctcgacccaggccggcaccgccttcgtgttcggctttctggccggcggcgaccagccctatccggtcagcaatccgggcgcgggcttcatcttcgccttccgcgtgctgccggtgatcctggtggtctgcgccctgtcggcgctgctgtggcactggaagattctcaagtggctggctcagggcttcggctttgtgttccagaagacgctgggcctgcgcggcccgccggccctggccaccgccgcgaccatcttcatgggtcaggtcgaggggccgatcttcatccgcgcctatctcgacaagctgagccgctcggaactcttcatgctgatcgcggtcggcatggcctgcgtgtcgggctcgaccatggtcgcctacgccaccatcctggccgacgtcctgcccaacgccgccgcccacgtgctgaccgcctcgatcatctcggctccggccggcgtgctgctggcccggatcattgtgccgtccgatccgatggagaagagcgccgatcttgatctgtcgaccgaggacaagacctatggcagctcgatcgacgccgtgatgaagggcaccaccgacggcctgcagatcgcgctgaacgtcggcgccaccctgatcgtcttcgtggccctggccaccatggtcgacaaggtcctgggcgccttcccgccggtgggcggcgagccgctgagcatcgcgcgcggcctgggcgtggtcttcgcgccgctggcctggtcgatgggcatcccgtggaaagaagcgggcacggccggcggtctgctgggcgtgaagctgatcctgaccgagttcaccgccttcatccagctgtccaaggtgggcgaagccctgctggacgaacgcacccggatgatcatgacctacgctctgtgcggtttcgccaatatcggctcggtcggcatgaacgtcgccggcttctcggtgctggtgccccagcgccggcaggaagtgctgggcctggtctggaaggcgatgatggccggcttcctggccacctgcctgaccgcctcgctggtcggcctgatgccgcgaagcctgtttgggctgtaa

SEQ ID NO: 114 - DNA, 1251 bp, Escherichia coli
  misc_feature 1..1251: note = yeiJ - Escherichia coli K-12 W3110 (AAC75222; JW2148)
  source 1..1251: mol_type = genomic DNA
atggatgtcatgagaagtgttctgggaatggtggtattgctgacgattgcgtttttactgtcagtaaacaagaagaagatcagcctgcgtaccgttggcgcggcgttagtgttacaggtcgtgattggcggcattatgctttggttaccgccagggcgttgggtcgctgaaaaagtcgcttttggcgtgcataaagtgatggcgtacagcgacgcgggtagcgcatttatcttcggttctctggtcggaccgaaaatggataccttatttgatggtgcaggatttatctttggtttcagggtgttaccggcaattatcttcgtcaccgcgctggtgagtattctctactacatcggtgtgatggggattttaattcgaattctcggcggtatcttccagaaagcattaaatatcagcaagatcgagtcattcgtcgcggtcaccaccattttcctcgggcaaaacgaaattccggcaatcgtcaaaccctttatcgatcgtctgaatcgcaatgaattatttacagcgatttgtagtggcatggcctcgattgctggttcgacaatgattggttacgccgcactgggcgtgcctgtggaatatctgctggcggcatcattaatggcgatccctggcgggatcttgtttgcccgcctgttaagcccggcaacggaatcttcgcaggtttcctttaataacctctctttcaccgaaacaccgccaaaaagcattattgaagccgctgcgacaggggcaatgaccgggctgaaaatcgccgcaggtgtggcaacagtggtgatggcatttgttgcaataattgcgttgattaacggtattatcggcggcgttggtggctggtttggttttgaacatgcctcgctggagtccattttaggttacctgctggctccactggcgtgggtgatgggtgtggactggagtgatgcgaatcttgccgggagtttgattggacagaaactggcaataaatgaatttgtcgcttatctcaatttctcaccctatctgcaaacggctggcactctcgatgctaaaactgtggcgattatttccttcgcgttgtgcggtttcgctaactttggttctatcggggtggtggtgggggcgttttctgcggttgcgccacaccgtgcgccggaaatcgcccagcttggtttacgggcgctggcggcggcgacgctttccaacttgatgagtgcgaccattgccgggttctttattggtttagcttga

SEQ ID NO: 115 - DNA, 1251 bp, Escherichia coli
  misc_feature 1..1251: note = yeiM - Escherichia coli K-12 W3110 (AAC75225; JW2151)
  source 1..1251: mol_type = genomic DNA
atggatataatgagaagtgttgtggggatggtggtgttactggcaatagcatttctgttgtcagtgaataaaaagagcatcagtttgcgcacggttggagccgcactgctgctgcaaatcgctattggtggcatcatgctctacttcccaccgggaaaatgggcagtagaacaggcggcattaggcgttcataaagtgatgtcttacagtgatgccggtagcgccttcatttttggttcgctggttgggccgaaaatggatgtcctgtttgacggtgcgggttttatcttcgcctttcgcgtacttccggcgattattttcgttactgcgctcatcagtctgctgtactacattggcgtgatggggctgctgattcgcatccttggcagcattttccagaaagccctcaacatcagcaaaatcgaatcttttgttgcggttactactattttcctcgggcaaaatgagatcccggcgatcgttaaaccgtttatcgatcgcatgaatcgcaacgagttgtttaccgcaatttgtagcgggatggcgtccattgctggttcgatgatgattggttatgccggaatgggcgtaccaattgactacctgttagcggcatcgctgatggcgatccctggcgggattttgtttgcacgtattcttagcccggcaaccgagccttcgcaggtcacatttgaaaatctgtcgttcagcgaaacgccgccaaaaagctttatcgaagcggcggcgagcggtgcgatgaccgggctaaaaatcgccgctggtgtggcgacggtggtaatggcgtttgtcgcaattattgcgctgatcaacggcattatcggcggaattggcggctggtttggtttcgccaatgcctctctggaaagtatttttggctatgtgctggcaccgctggcgtggatcatgggtgtggactggagtgatgccaatcttgcgggtagcctgattgggcagaaactggcgattaacgaattcgtcgcttacctgagtttctccccatacctgcaaacgggcggcacgctggaagtgaaaaccattgcgattatctcctttgcgctttgtggttttgctaactttggttctatcggtgttgtcgttggcgcattttcggctatttcgccaaaacgcgcgccggaaatcgcccagcttggtttacgggcgctggcagcagcaacgctttccaacctgatgagtgcgactattgccgggttctttattggtctggcgtaa

SEQ ID NO: 116 - DNA, 1254 bp, Haemophilus influenzae
  misc_feature 1..1254: note = HI0519 - Haemophilus influenzae Rd KW20 serotype d(AAC22177)
  source 1..1254: mol_type = genomic DNA
atgagtgtgttaagcagcattttgggaatggtcgtattaatcgctattgccgtgttactttctaataatcgtaaagcgattagtattcgaaccgtagtaggggcgttagcaatccaagtaggatttgccgcccttattttatatgtgccagcaggtaaacaagcgttgggtgccgctgcggatatggtatccaatgttattgcctatggtaatgacgggattaatttcgttttcggcggattggcagatccaagtaaaccatccggtttcatttttgcagtgaaagtattaccgattatcgtgttcttctctggcttaatttctgtgctttactatctcggcattatgcaagtcgtgattaaagtattaggtggcgcattacaaaaagcattgggtacgtcaaaagcggaatcaatgtcagcggcggcgaatatcttcgtcggtcaaactgaagcaccattagttgttcgcccttacattaaaaatatgacccaatctgaattatttgccattatggtgggtggtacagcgtctatcgcgggttcagtaatggcaggttatgctggaatgggcgtgccattgacatacttaatcgctgcgtcatttatggcggcaccagcaggtttattatttgcgaaattaatgttcccacaaaccgaacaattcacagataaacaaccagaagacaatgattcagaaaaaccaactaacgtacttgaagcaatggcgggcggtgcgagtgcaggtatgcaacttgcgttaaacgtaggtgcaatgttaatcgcattcgttggtttaattgcattaattaatggtattttaagtggcgtaggcggatggttcggctatggcgacttaaccttacaatctatctttggtttaatttttaaaccattagcatacttaatcggtgtaactgatggtgctgaagcaggtattgcaggacaaatgatcgggatgaaattagcggttaatgaatttgtgggttatcttgaatttgcaaaatatttacaaccagattctgcaattgtattaactgaaaaaaccaaagcgattattactttcgcactttgtggttttgctaacttcagctcaattgcaatcttaattggtggtttaggtggtatggcaccaagccgtcgtagtgatgttgctcgtttaggtatcaaagccgttatcgctggtactctcgctaacttaatgagtgcaactattgctggtttatttatcggcttaggtgctgcagcactttaa

SEQ ID NO: 117 - DNA, 1257 bp, Helicobacter pylori
  misc_feature 1..1257: note = nupC (HP1180) - Helicobacter pylori 26695 (AAD08224)
  source 1..1257: mol_type = genomic DNA
atgatttttagctctctttttagtgttgtagggatggcggtgctttttcttattgcttgggtgttttctagcaataaaagggctattaattatcgcacgattgtcagtgcctttgtgattcaagtggctttaggggcgttggctttatatgtgcctttgggtagggaaatgctgcaaggcttagccagcggcatacaaagcgtgatttcttacggctatgagggggtgcgttttttatttggcaatctcgctccaaacgctaagggcgatcaagggataggggggtttgtctttgcgatcaatgttttagcgatcattatcttttttgctagcttgatttcacttctatattatttaaaaatcatgcctttatttatcaatctcatcggtggggcgttgcaaaaatgcttaggcacttctagagcagaaagcatgagtgcagcggctaatatttttgtagcgcacaccgaagcgcccttagtcattaaaccttatttgaaaagcatgagcgattcagagatttttgcggtcatgtgcgtgggcatggctagcgttgcggggcctgtgttagccgggtatgcgagcatgggcattcctttgccttatttgatcgccgcttcgtttatgtccgctcctggggggttgttgttcgctaaaatcatttacccacaaaacgaaaccatttctagccatgcagatgtttctatagaaaagcatgtcaatgccatagaagctatcgctaatggggcaagcacagggctaaatttagccttgcatgtgggagcgatgcttttagcctttgtggggatgctcgcgctcattaacgggcttttaggggttgtagggggttttttaggcatggagcatttgtctttagggttgattttaggcacgctcttaaaacccttagcctttatgttaggcattccttggagccaggccgggattgccggagaaatcataggcattaaaatcgcgctcaatgaatttgtgggctatatgcagttattgccttatttgggcgataaccctcctttaatcttgagcgagaaaactaaagcgatcatcacttttgcgttgtgcgggtttgctaatttaagctcagtcgctatgctcattggagggcttggcagtttagtgcctaaaaagaaggatctcattgtaaggcttgctttaaaagcggtgcttgtaggcacgctttctaatttcatgagcgcgactatcgccgggttattcatagggctaaacgctcattaa

SEQ ID NO: 118 - DNA, 1230 bp, Staphylococcus aureus
  misc_feature 1..1230: note = nupC (SA0600) - Staphylococcus aureus subsp. aureus N315 (BAB41833)
  source 1..1230: mol_type = genomic DNA
atgtttttattaatcaacattattggtctaattgtatttcttggtattgcggtattattttcaagagatcgcaaaaatatccaatggcaatcaattgggatcttagttgttttaaacctgtttttagcatggttctttatttattttgattggggtcaaaaagcagtaagaggagcagccaatggtatcgcttgggtagttcagtcagcgcatgctggtacaggttttgcatttgcaagtttgacaaatgttaaaatgatggatatggctgttgcagccttattcccaatattattaatagtgccattatttgatatcttaatgtactttaatattttaccgaaaattattggaggtattggttggttactagctaaagtaacaagacaacctaaattcgagtcattctttgggatagaaatgatgttcttaggaaatactgaagcattagccgtatcaagtgagcaactaaaacgtatgaatgaaatgcgtgtattaacaatcgcaatgatgtcaatgagctctgtatccggagctattgtaggtgcgtatgtacaaatggtaccaggagaactggtactaacggcaattccactaaatatcgttaacgcgattattgtgtcatgcttgttgaatccagtaagtgttgaagagaaagaagatattatttacagtcttaaaaacaatgaagttgaacgtcaaccattcttctcattccttggagattctgtattagcagcaggtaaattagtattaatcatcatcgcatttgttattagttttgtagcgttagctgatctatttgatcgttttatcaatttgattacaggattgatagcaggatggataggcataaaaggtagtttcggtttaaaccaaattttaggtgtgtttatgtatccatttgcgctattactcggtttaccttatgatgaagcgtggttggtagcacaacaaatggctaagaaaattgttacaaatgaatttgttgttatgggtgaaatttctaaagatattgcatcttatacaccacaccatcgtgcggttattacaacattcttaatttcatttgcaaacttctcaacgattggtatgattatcggtacattgaaaggcattgttgataaaaagacatcagactttgtatctaaatatgtacctatgatgctattatcaggtatcctagtttcattattaacagcagctttcgttggtttatttgcatggtaa

SEQ ID NO: 119 - DNA, 1230 bp, Staphylococcus aureus; identical to SEQ ID NO: 118
  misc_feature 1..1230: note = nupC (SAV0645) -Staphylococcus aureus subsp. aureus Mu50 (BAB56807)
  source 1..1230: mol_type = genomic DNA

SEQ ID NO: 120 - DNA, 1203 bp, Streptococcus pyogenes
  misc_feature 1..1203: note = nupC (SpNupC) - Streptococcus pyogenes SF370 serotype M1 (AAK34582)
  source 1..1203: mol_type = genomic DNA
atgcaatttatttatagtattattggtattttattggtattaggaattgtgtatgcaatttctttcaatcgtaagagtgtttctctaagtttaattggaaaagctcttatcgttcaattcattattgcgctaatcttagtacgtatcccactaggccaacaaattgttagtgttgtttcaactggagttactagcgtaatcaactgtggtcaagctggtttaaattttgtgtttgggtcattagcagatagtggcgcaaaaactggttttattttcgctattcaaacgcttggtaatattgttttcttatctgccctagttagtctactttattatgtaggaatccttggatttgtagtaaaatggataggtaagggcgttggtaaaattatgaaatcctcagaggttgagagttttgttgctgtagctaatatgtttcttggtcaaacagacagtccaatcttggttagcaaatacctaggtcgtatgactgatagtgagataatggttgtgttggtatcaggtatgggaagtatgtcagtttctattcttggtggctatattgcattaggcattccaatggaatatctcttgattgcttcaacaatggttcctattggcagtattctcattgctaaaatcttattgcctcaaacagaacctgttcaaaaaattgatgacattaagatggataataaaggtaataacgccaatgtgattgatgcaatcgctgagggtgcaagcacaggtgcacaaatggctttctcaattggtgctagtttgattgcctttgttggtttagtttctttgattaatatgatgttaagtggattgggaatccgcttagaacaaatcttttcatatgtttttgctccatttggttttcttatgggatttgaccacaaaaacattcttctagaaggaaaccttcttggaagtaagttgattttaaatgagtttgtttcgttccaacaattgggtcacctaatcaaatctttagattatcgtacagcattggtagcaactatttcactctgtggttttgctaatttatcaagtttaggtatttgtgtttcaggtattgctgttctttgcccggagaaacgtagcaccctagctcgacttgttttccgtgcaatgattggtggtattgctgtaagtatgcttagcgcctttatcgtcggtattgtaactctattctaa

SEQ ID NO: 121 - DNA, 1257 bp, Vibrio cholerae
  misc_feature 1..1257: note = nupC (VC2352) - Vibrio cholerae O1 biovar El Tor N16961 (AAF95495)
  source 1..1257: mol_type = genomic DNA
atgagcctgtttatgagcctcatcggcatggcagttctgctaggaatcgcagttctactgtcaagtaaccgtaaagctatcaatctaagaactgtgggtggcgcttttgctatccaattttcactgggtgcatttattctgtatgtgccttggggccaagagctacttcgtggcttttcggatgccgtatcgaatgttattaactacggtaacgatggtacttcattcctcttcggtggactggtatcaggcaaaatgtttgaagtgtttggcggcggcggtttcattttcgcattccgcgtactaccaacactgatcttcttctcagcactgatttctgtactgtactacttgggtgttatgcaatgggttatccgcattcttggcggtggtctgcaaaaagcactgggtacatcacgcgcggaatctatgtctgcggctgcaaacattttcgtgggtcaaactgaagcaccattagttgttcgtccattcgttccaaaaatgactcaatctgagctgtttgcggtaatgtgtggtggcttggcttctatcgcaggtggtgtacttgcgggttacgcttcaatgggcgttaagatcgaatacttggtagcggcgtcattcatggcggcaccgggtggtctgctgttcgcaaaactgatgatgcctgaaactgaaaaaccacaagacaatgaagacattactcttgatggtggtgacgacaaaccggctaacgttatcgatgcggctgctggcggtgcttctgctggtctgcaacttgctctgaacgttggtgcaatgttgattgcctttatcggtttgattgctctgatcaacggtatgttgggtggcatcggtggttggttcggtatgcctgaactgaaactggaaatgctactgggctggttgtttgcgcctctggctttcctgatcggtgttccttggaacgaagcaactgttgcgggtgagttcatcggtctaaaaaccgttgctaacgaattcgttgcttactctcagtttgcgccttacctgactgaagcggcaccagtggttctgtctgagaaaaccaaagcgatcatctctttcgctctgtgtggttttgcgaacctttcttctatcgcaattctgcttggtggtttgggtagcttggcacctaagcgtcgtggcgacatcgctcgtatgggggtcaaagcggttatcgcaggtactctatctaacctgatggcagcgaccatcgctggcttcttcctctctttctaa

SEQ ID NO: 122 - DNA, 1218 bp, Vibrio cholerae
  misc_feature 1..1218: note = nupC (VC1953) - Vibrio cholerae O1 biovar El Tor N16961 (AAF95101)
  source 1..1218: mol_type = genomic DNA
ttgggcggcgttatgtcatcactcctcggtatgggcgcaattttgctggttgcgtggctattttctaccaatagaaaaaatatcaacttgcgtacagtttctttagcgttactgctgcaaatcttcttcgccttactggtgctgtatgtacctgcgggtaaagaggcactcaatcgtgtgacgggcgcggtgtcacaactgatcaactatgggcaagatggtatcggttttgtgtttggtggcctcgccaatggcagcgtaggttttgtgtttgcgattaatgtccttggcatcatcattttcttctctgcactgatttctggcctttaccatttaggcatcatgccgaaagtgattaacctcatcggtggtggtttacagaaattgcttggcacaggccgtgcagaatccctttctgctaccgcaaacattttcgtgggtatgattgaagcgccgctggtggtgaaaccttatcttcataaaatgaccgattcgcaattctttgcagtgatgacgggcggcttagcgtcggttgctggcggtactttggttggttatgcctctttaggtgtggaattgaactatctgatcgcggcggctttcatgtctgcccctgcgggtcttttgatggcaaaaatcatgttgccagaaaccgaacacgtcgatgccgcgattgcgcaagatgagttggatctgccgaaatccactaacgtcgtcgaagcgattgcggatggcgcgatgtcgggtgtgaaaattgctgttgcggtaggggcgactttgctcgctttcgtgagtgtgattgctctgttaaacggcttgctcggttggtttggtggctggtttggcatcgagctaagctttgaactgatcatggggtatgttttcgctccggtagcttggctgattggtattccatggcatgaggcgatcacggcaggctcgctgattggtaacaaagtggtggtgaacgagtttgtcgctttcattcaactgattgaagtgaaagagcaattgagtgcgcattcacaagcgatcgtgactttcgcgctgtgcggttttgcgaatatttctaccatggcgattttgattggtggtttgggtagccttgtacctgaacgtcgctcttttatctcccaatacggcttccgtgcgattggcgcaggcgtattagctaacctaatgagtgcatcgatcgctggagtgattttgtctttgtga

SEQ ID NO: 123 - DNA, 1209 bp, Vibrio cholerae
  misc_feature 1..1209: note = nupC (VCA0179) - Vibrio cholerae O1 biovar El Tor N16961 (AAF96092)
  source 1..1209: mol_type = genomic DNA
atggcgattttgtttggaatcatcggtgttacggtactgatcttatgcgcgtatctgctctctgaaagccgcagtgcgattaattggaaaaccatttcccgagccttgttgttgcaaattggttttgcggctcttgtgctttatttcccattggggcaaaccgcgctaagcagcttgagtaatggggtttctggtttgcttggttttgccgatgtcggcattcgctttctgtttggtgatcttgccgatacgggctttatttttgctgttcgtgtattacctatcatcatcttcttcagtgcgctgatttctgccctttattaccttggtgtgatgcaaaaagtgatcgccctgatcggcggtggcattcaacgcttcttaggcaccagtaaggcggaatcactggtcgcgacaggcaatattttcctatcacaaggcgaatcgccacttttggtgcgccccttccttgccaatatgacacgctctgaactgtttgcggtcatggcgggcggtatggcatcggtagcaggctctgtgctgggtggttacgcaggtttaggggttgagctgaaatacctgattgcagcgagtttcatggcggcgccgggcagtttaatgatggcgaaaatcatcgttcctgagcgtggtgtgccaatcgatcaaagccaagtcgagttagataaagcgcaagacagcaacttgattgatgctctcgctagcggtgcgatgaatggtatgaaagtcgccgttgcagtgggcactatgttgattgcgttcgtcagcgtgatcgctatggtcaacactggccttgaaaatctgggcgatctggttgggtttagcggcattaccttacaagccatgttcggttatctgtttgctcctctggcatgggtgattggcattccaagtcacgaagtgctggcggcaggttcctacatcggtcagaaagtggtgatgaacgaatttgtggctttcattgactttgttgagcataaagcgctgctttctgagcatagccaagtcatcatcacgtttgcattgtgtggctttgccaacattggctctatcgcgatccaattaggctccattggcgtgatagcccctgagcgccgctcggaagtggcgaacctaggcataaaagcggtcattgctggcactttagccaacctaatgagcgcttgcttagcggggattttcatctcgctataa

SEQ ID NO: 124 - DNA, 1278 bp, Escherichia coli
  misc_feature 1..1278: note = yegT - Escherichia coli K-12 W3110 (P76417; JW2085)
  source 1..1278: mol_type = genomic DNA
atgaaaacaacagcaaagctgtcgttcatgatgtttgttgaatggtttatctggggcgcgtggtttgtgccattgtggttgtggttaagtaaaagcggttttagtgccggagaaattggctggtcgtatgcctgtaccgccattgcggcgatcctgtcgccaattctggttggctccatcactgaccgctttttctcggcgcaaaaagtgctggcggtattgatgttcgcaggcgcgctgctgatgtatttcgctgcgcaacagaccacttttgccgggttcttcccgttactgctggcctactcgctaacctatatgccgaccattgcgctgactaacagcatcgcttttgccaacgtgccggatgttgagcgtgatttcccgcgcattcgtgtgatgggcactatcggctggattgcctccggtctggcatgtggtttcttgccgcaaatactggggtatgccgatatctcaccgactaacatcccgctgctgattaccgccggaagttctgctctgctcggtgtgtttgcgtttttcctgcccgacacgccaccaaaaagcaccggcaaaatggatattaaagtcatgctcggcctggatgcgctgatcctgctgcgcgataaaaacttcctcgtctttttcttctgttcattcctgtttgcgatgccactagcgttctattacatctttgccaacggttatctgaccgaagttggcatgaaaaacgccaccggctggatgacgctcggccagttctctgaaatcttctttatgctggcattgccgtttttcactaaacgctttggtatcaaaaaggtattattgcttggtctggtcaccgctgcgatccgctatggcttctttatttacggtagtgcggatgaatatttcacctacgcgttactgttcctcggtattttgcttcacggcgtaagttacgatttttactacgttaccgcttacatctatgtcgataaaaaagcccccgtgcatatgcgtaccgctgcgcaggggctgatcacgctctgctgccagggcttcggcagtttgctcggctatcgtcttggcggtgtgatgatggaaaagatgttcgcttatcaggaaccggtaaacggactgactttcaactggtccgggatgtggactttcggcgcggtgatgattgccattatcgccgtgctgttcatgatttttttccgcgaatccgacaacgaaattacggctatcaaggtcgatgatcgcgatattgcgttgacacaaggggaagttaaatga

SEQ ID NO: 125 - DNA, 1257 bp, Escherichia coli
  misc_feature 1..1257: note = nupG - Escherichia coli K-12 W3110 (P09452; JW2932)
  source 1..1257: mol_type = genomic DNA
atgaatcttaagctgcagctgaaaatcctctcttttctgcagttctgtctgtggggaagttggctgacgaccctcggctcctatatgtttgttaccctgaagtttgacggtgcttctattggcgcagtttatagctcactgggtatcgcagcggtctttatgcctgcgctgctggggattgtggccgacaaatggttaagtgcgaaatgggtatatgccatttgccacaccattggcgctatcacgctgttcatggcggcacaggtcacgacaccggaagcgatgttccttgtgatattgattaactcgtttgcttatatgccaacgcttgggttaatcaacaccatctcttactatcgcctgcaaaatgccgggatggatatcgttactgacttcccgccaatccgtatctggggcaccatcggctttatcatggcaatgtgggtggtgagcctgtctggcttcgaattaagccacatgcagctgtatattggcgcagcactttccgccattctggttctgtttaccctgactctgccgcatattccggttgctaaacagcaagcgaatcagagctggacaaccctgctgggcctcgatgcattcgcgctgtttaaaaacaagcgtatggcaatcttctttatcttctcaatgctgctgggcgcggaactgcagattaccaacatgttcggtaataccttcctgcacagcttcgacaaagatccgatgtttgccagcagctttattgtgcagcatgcgtcaatcatcatgtcgatttcgcagatctctgaaaccctgttcattctgaccatcccgttcttcttaagccgctacggtattaagaacgtaatgatgatcagtattgtggcgtggatcctgcgttttgcgctgtttgcttacggcgacccgactccgttcggtactgtactgctggtactgtcgatgatcgtttacggttgcgcattcgacttcttcaacatctctggttcggtgtttgtcgaaaaagaagttagcccggcaattcgcgccagtgcacaagggatgttcctgatgatgactaacggcttcggctgtatcctcggcggcatcgtgagcggtaaagttgttgagatgtacacccaaaacggcattaccgactggcagaccgtatggttgattttcgctggttactccgtggttctggccttcgcgttcatggcgatgttcaaatataaacacgttcgtgtcccgacaggcacacagacggttagccactaa

SEQ ID NO: 126 - DNA, 1257 bp, Escherichia coli
  misc_feature 1..1257: note = xapB - Escherichia coli K-12 W3110 (P45562; JW2397)
  source 1..1257: mol_type = genomic DNA
atgagcatcgcgatgcgcttaaaggtaatgtcctttttgcaatattttatctgggggagctggctggttaccctcggctcttacatgattaatactcttcatttcaccggcgctaatgttggcatggtttacagttccaaagggatcgccgcgattattatgcctggtataatggggatcatcgcagacaaatggctgcgcgcagaacgtgcatacatgctgtgtcacctggtgtgtgcgggcgtacttttttatgcggcatccgtaactgatccggatatgatgttttgggtgatgttagtcaatgcgatggcgtttatgccgactattgcgttatcgaacagcgtctcttattcctgtcttgcccaggcagggcttgacccggtgaccgctttcccgcccattcgcgtttttggtacggtggggttcattgtcgcgatgtgggcagtaagcctgctgcatctggaattgagtagtctgcagctgtatatcgcgtccggtgcgtcattgctgctgtcggcttatgcgctgactttgccgaagattccggttgcggagaaaaaagcgaccacatcgcttgccagcaagctgggtctggatgccttcgtgctgtttaaaaatccacgcatggccatctttttcctctttgccatgatgctgggtgcggtactgcaaattaccaacgtttttggtaatccgttcctacatgatttcgcccgtaacccggagtttgctgacagttttgtggtgaaatatccctccattttactgtcagtttcacagatggcagaagtgggctttatactgactatcccattctttttaaagcgatttggcattaaaaccgtcatgctgatgagtatggtggcctggacgctgcgctttggcttcttcgcctatggcgatccgtcaacaaccggatttattttgctgctgctgtcgatgattgtttatggctgtgcattcgatttcttcaatatttctggttcggtatttgtcgaacaggaagttgattccagcattcgtgccagcgcgcaggggctctttatgaccatggtaaatggtgtcggcgcatgggttggctcgattctgagtggcatggcagtagattacttttcggtggatggcgtaaaagactggcaaactatctggctggtgtttgcaggatatgctctttttctcgcagtgatatttttctttgggtttaaatataatcatgaccctgaaaagataaagcatcgagcggtgactcattaa

SEQ ID NO: 127 - DNA, 1242 bp, Caulobacter crescentus
  misc_feature 1..1242: note = CC1628 - Caulobacter crescentus CB15 (AAK23606)
  source 1..1242: mol_type = genomic DNA
atggggacgagtttccgtctgttcgtgatgatggtgctgcagctggcgatctggggcgcctgggcgcccaagatcttcccctacatgggcatgctgggcttcgcgccctggcagcagtcgctggtcggcagcgcctggggcgtggcggcgctggtgggcatcttcttctcgaatcagttcgccgaccggaacttctcggccgagcggttcctggcggtcagccacctgatcggcggcgtggcgctgctgggcacggccttctcgacggagttctggccgttctttgcctgttacctcgttttcagcctggtctatgtgccgacgctgtcggtcaccaactcgatcgccttcgccaatctgcgcgatccggcggccggcttcggcggggtgcggatgggcggaaccgtcggctgggtgctggtcagctggcccttcgtgttcctgctgggcgcccaagcgacggtggagcaggtccgctggatcttcctggtggcggcgatcgtctccttcgttttcgccggttacgctctgaccctgccgcacacgccgccgcgcaaggccgatgacgctgtcgacaagctggcctggcgacgggcgttcaagctactgggcgcgcccttcgtgtttgtcctctttgtcgtgaccttcatcgattccgtgatccacaacggctacttcgtgatggccgacgccttcctgaccaaccgggtcgggatcgcgggcaatctcagcatggtcgtgctgagcctgggccaggtggccgaaatcatcaccatgctgctgttgggccgcgtgctggccaagctgggctggaaggtcaccatgatcgtcggcgtgctgggccacgccgcgcgctttgcggtcttcgcctacttcgccgacagcgtgccggtcatcgtggcggtgcagctgctgcacggcgtctgctacgccttcttcttcgccacggtttacatcttcgtcgacgccgtcttcccgaaagatgtccgctccagcgcgcagggtctgttcaacttgctgatcctgggcgtcggcaatgtggccgccagcttcatcttccccgcgctgatcggtcgcctgaccaccgatgggtccgtcgactacacgacgctgttcctcgtgccgaccgccatggctttggcggcggtctgcctgctggcgctgttcttccggccgcccacgcggggacctgtttcggaggcggattccgcttcatccgccgccagttcggcccaagcctag

SEQ ID NO: 128 - DNA, 1260 bp, Escherichia coli
  misc_feature 1..1260: note = codB - Escherichia coli K-12 W3110 (P25525; JW0327)
  source 1..1260: mol_type = genomic DNA
gtgtcgcaagataacaactttagccaggggccagtcccgcagtcggcgcggaaaggggtattggcattgacgttcgtcatgctgggattaaccttcttttccgccagtatgtggaccggcggcactctcggaaccggtcttagctatcatgatttcttcctcgcagttctcatcggtaatcttctcctcggtatttacacttcatttctcggttacattggcgcaaaaaccggcctgaccactcatcttcttgctcgcttctcgtttggtgttaaaggctcatggctgccttcactgctactgggcggaactcaggttggctggtttggcgtcggtgtggcgatgtttgccattccggtgggtaaggcaaccgggctggatattaatttgctgattgccgtttccggtttactgatgaccgtcaccgtcttttttggcatttcggcgctgacggttctttcggtgattgcggttccggctatcgcctgcctgggcggttattccgtgtggctggctgttaacggcatgggcggcctggacgcattaaaagcggtcgttcccgcacaaccgttagatttcaatgtcgcgctggcgctggttgtggggtcatttatcagtgcgggtacgctcaccgctgactttgtccggtttggtcgcaatgccaaactggcggtgctggtggcgatggtggcctttttcctcggcaactcgttgatgtttattttcggtgcagcgggcgctgcggcactgggcatggcggatatctctgatgtgatgattgctcagggcctgctgctgcctgcgattgtggtgctggggctgaatatctggaccaccaacgataacgcactctatgcgtcgggtttaggtttcgccaacattaccgggatgtcgagcaaaaccctttcggtaatcaacggtattatcggtacggtctgcgcattatggctgtataacaattttgtcggctggttgaccttcctttcggcagctattcctccagtgggtggcgtgatcatcgccgactatctgatgaaccgtcgccgctatgagcactttgcgaccacgcgtatgatgagtgtcaattgggtggcgattctggcggtcgccttggggattgctgcaggccactggttaccgggaattgttccggtcaacgcggtattaggtggcgcgctgagctatctgatccttaacccgattttgaatcgtaaaacgacagcagcaatgacgcatgtggaggctaacagtgtcgaataa

SEQ ID NO: 129 - DNA, 1656 bp, Corynebacterium sp.
  misc_feature 1..1656: note = mctC Corynebacterium
  source 1..1656: mol_type = genomic DNA
atgaattccactattctccttgcacaagacgctgtttctgagggcgtcggtaatccgattcttaacatcagtgtcttcgtcgtcttcattattgtgacgatgaccgtggtgcttcgcgtgggcaagagcaccagcgaatccaccgacttctacaccggtggtgcttccttctccggaacccagaacggtctggctatcgcaggtgactacctgtctgcagcgtccttcctcggaatcgttggtgcaatttcactcaacggttacgacggattcctttactccatcggcttcttcgtcgcatggcttgttgcactgctgctcgtggcagagccacttcgtaacgtgggccgcttcaccatggctgacgtgctgtccttccgactgcgtcagaaaccagtccgcgtcgctgcggcctgcggtaccctcgcggttaccctcttttacttgatcgctcagatggctggtgcaggttcgcttgtgtccgttctgctggacatccacgagttcaagtggcaggcagttgttgtcggtatcgttggcattgtcatgatcgcctacgttcttcttggcggtatgaagggcaccacatacgttcagatgattaaggcagttctgctggtcggtggcgttgccattatgaccgttctgaccttcgtcaaggtgtctggtggcctgaccacccttttaaatgacgctgttgagaagcacgccgcttcagattacgctgccaccaaggggtacgatccaacccagatcctggagcctggtctgcagtacggtgcaactctgaccactcagctggacttcatttccttggctctcgctctgtgtcttggaaccgctggtctgccacacgttctgatgcgcttctacaccgttcctaccgccaaggaagcacgtaagtctgtgacctgggctatcgtcctcattggtgcgttctacctgatgaccctggtccttggttacggcgctgcggcactggtcggtccagaccgcgtcattgccgcaccaggtgctgctaatgctgctgctcctctgctggccttcgagcttggtggttccatcttcatggcgctgatttccgcagttgcgttcgctaccgttctcgccgtggtcgcaggtcttgcaattaccgcatccgctgctgttggtcacgacatctacaacgctgttatccgcaacggtcagtccaccgaagcggagcaggtccgagtatcccgcatcaccgttgtcgtcattggcctgatttccattgtcctgggaattcttgcaatgacccagaacgttgcgttcctcgtggccctggccttcgcagttgcagcatccgctaacctgccaaccatcctgtactccctgtactggaagaagttcaacaccaccggcgctgtggccgctatctacaccggtctcatctccgcgctgctgctgatcttcctgtccccagcagtctccggtaatgacagcgcaatggttccaggtgcagactgggcaatcttcccactgaagaacccaggcctcgtctccatcccactggcattcatcgctggttggatcggcactttggttggcaagccagacaacatggatgatcttgctgccgaaatggaagttcgttccctcaccggtgtcggtgttgaaaaggctgttgatcactaa

SEQ ID NO: 130 - DNA, 1521 bp, Virgibacillus sp.
  misc_feature 1..1521: note = putP_6 - Virgibacillus sp.
  source 1..1521: mol_type = genomic DNA
atggatcttacgacattaataacttttatagtatatctactagggatgttggcgattggcctcatcatgtattatcgaaccaataatttatcagattatgttcttggtggacgtgatcttggtccaggcgtagctgcattgagtgctggtgcatcggatatgagtggttggctgttattaggtttgcctggagcgatttatgcatctggtatgtctgaagcttggatggggatcgggttagctgtaggtgcttatttaaattggcaatttgtagctaagcgattacgcgtttataccgaggtatcaaataattccattacgatcccagattattttgaaaatcggtttaaagataactcacatattcttcgtgttatatctgctatcgtaattttgttattcttcactttttatacatcttcaggaatggttgcaggagcaaaattatttgaggcttcattcggtctccaatacgaaactgctctgtggattggtgcggttgtagttgtatcttatacgttacttggaggatttctagcggttgcatggacagactttattcaaggtattcttatgttccttgcactaattgttgttccaatcgtcgcattagatcaaatgggtggctggaatcaagcggtacaagctgttggtgaaattaatccttcccacctcaatatggttgaaggtgttggaataatggcaattatttcatcacttgcttggggcttaggttattttggacagccacatattattgttcgttttatggcattacgttcggcgaaagatgttccgaaagcgaaatttattggaacagcttggatgattttaggactttatggagcaatctttactggttttgtaggactagcatttatcagtacacaagaagtaccgattctgtctgaattcgggattcaagtagttaatgagaatggtttacaaatgttagccgatcctgaaaagatatttattgctttctcccaaatactattccatccagtagttgccggtatcttactagcggcaatcttgtctgcaattatgagtaccgttgattcacagttacttgtatcatcttcagcggttgcagaagatttctataaagctattttccgtaaaaaagctactggtaaagagcttgtttgggttggacgtattgctacagtgataattgcgattgttgctttaattattgcaatgaacccagatagctctgtattggatctagttagttatgcatgggctggatttggtgcagcatttggaccaattatcatcttgtcattattctggaagagaatcacaagaaatggtgcactagcgggtatcattgtaggtgccattacggtaattgtatggggagactttctatctggaggtatctttgacctctacgaaattgttccaggctttatcttaaatatgattgtcaccgttattgtgagtcttatcgataaaccgaatccagatttagaagctgactttgatgaaaccgtagaaaaaatgaaagaataa

SEQ ID NO: 131 - DNA, 1323 bp, Lactobacillus johnsonii
  misc_feature 1..1323: note = cbsT1 - Lactobacillus johnsonii
  source 1..1323: mol_type = genomic DNA
atgtcgaccacaccgacacagccatcatcacgaaaacaggctgtttacccgtacttgatcgtgctgtcgggcatcgtcttcacggccatcccggtatcgctggtctgcagttgcgcaggtatcttcttcacgcctgtcagcagctacttccatgttcccaaggccgcattcaccggatatttcagcatattcagcatcaccatggtcgccttcctgccggtggccggatggctgatgcaccgctacgatctgcgcatcgtactgaccgcaagcaccgtcctggctggactgggctgcctgggtatgtcccgatcatccgccatgtggcagttctatctatgcggagtggttctgggaatcggcatgccggccgtcctctatctgtcagtgccaacactcatcaacgcctggttccgcaagcgggtcgggttcttcatcggcctgtgcatggccttcaccggcataggcggcgtgatcttcaaccagataggcaccatgatcatcagatccgcccctgatggatggaggcggggatatctggttttcgctattctcatcctggtgatcaccctgcccttcaccattttcgtcattcgcagcacacccgaacagatgggtctgcatccctacggcgccgaccaggagcctgatgcagctgagacggccaccaatagtgcaggcaccgggagcaaagaccaaaagagtcctgagcctgcagcgtcaaccgtaggcatgactgcctcccaggccttgcgctcccctgccttctgggcgctggcgctcttctgcggtctgatcaccatgaatcagaccatttaccagttcctgccctcctacgcggcatccctgccatccatggcagcctacacgggactgatcgcctcctcctgcatggccggccaggccatcggcaagatcatcctgggcatggtcaacgacggcagcatcgtaggcggtctctgtctgggcatcggcggcggcattctcggcgtctgcctcatggtcgccttccccggattgcccgtgctcctcctgctgggagcctttgccttcggccttgtctacgcctgcactactgtgcagacaccaatcctggttacagcggtcttcggctcgcgcgactacaccaacatctatgcacgtatccagatggttgggtccctagcctcggccttcgcagctctcttctggggcgccatcgctgaccagccccacggctacatcatcatgttcggtctgagcatcctgatcatggttgtggccttgttcctaggcattatccctctgaaaggtacgcgcaagttgaccgatcagatcgcctga

SEQ ID NO: 132 - DNA, 1356 bp, Lactobacillus johnsonii
  misc_feature 1..1356: note = cbsT2 - Lactobacillus johnsonii
  source 1..1356: mol_type = genomic DNA
atgtctactgatgccgctactaaagataaagtagtaagcaagggctataaatacttcatggttttcctttgtatgttaacccaagctattccttatggaattgctcaaaacattcagcctttgtttatccaccctttagttaatactttccactttaccttagcatcgtacacattaatttttacgtttggtgcggtttttgcttcagttgcttctccatttattggtaaggcattagaaaaagttaacttccgactaatgtatttaattggtattggtctttctgctattgcctacgtaatttttggaattagtacaaaactacccggtttctatattgccgctatcatttgtatggttggttcaaccttttactccggccaaggtgttccctgggttattaaccactggttcccagcaaagggacgtggggctgccttaggaattgccttctgcggtggttctattggtaatatctttttacaaccagcaacccaagctattttaaaacactacatgacaggtaatactaagaccggtcatttaacctctatggcaccattctttatctttgccgtagctttattagtaatcggtgtaattatcgcctgcttcattagaacccctaagaaagacgaaattgttgtttctgatgcagaactagctgaaagcaagaaagctgaagccgcagccaaagctaaagagtttaaaggctggactagtaaacaagtgttacaaatgaaatggttctggattttcagccttggtttcttaatcattggtttaggcttagcttctttaaatgaagactatgccgccttccttgatactaagctttctttaaccgatgttggtttagttgggtcaatgtacggtgttggttgtttaatcggaaatatttctggtggtttcttatttgataaatttggtacagcaaaatcaatgacctatgctggttgtatgtatattttatctattctgatgatgatctttattagcttccagccatatggttcatctattagtaaggctgctggcattggctatgctatcttttgcggcttagctgtatttagttacatgtcaggcccagccttcatggcaaaagacctctttggttcaagagatcaaggtgtcatgcttggatacgttggtttagcttatgcaattggctatgccattggtgctccactatttgggattattaagggagcggcaagctttacagttgcttggtactttatgattgcctttgttgcaattggttttatcattttagtatttgccgttatccaaattaagagataccaaaagaaatacattgcagagcaagcagcaaaagctaatgctaaataa

SEQ ID NO: 133 - DNA, 1287 bp, Escherichia coli
  misc_feature 1..1287: note = amtB - Escherichia coli K-12 MG1655 (B0451; 945084)
  source 1..1287: mol_type = genomic DNA
atgaagatagcgacgataaaaactgggcttgcttcactggcgatgcttccgggactggtaatggctgcacctgcggtggccgataaagccgacaatgcgtttatgatgatttgtactgcgctggtgctgtttatgactattccggggattgccctgttttacggtgggttgattcgcggcaaaaacgtgctgtcgatgctgacgcaggtgacggtgacatttgcactggtctgtattctctgggtggtttacggttactcgctggcgtttggtgagggcaacaacttcttcggcaacattaactggttgatgctgaaaaacatcgaactgacggcggtgatgggcagcatttatcagtatatccacgtggcgtttcagggatcgtttgcctgcattaccgtcggcttgatagttggggcgctggcggaacgaatccgcttctcagctgtgttgattttcgtggtggtatggctgacgctctcttacattccgattgcgcatatggtgtggggcggtggtttgctggcttctcacggtgcgctggatttcgcgggtggcaccgtggtgcacattaacgccgcaatcgccggtctggtgggcgcgtatctgataggaaaacgcgtgggcttcggtaaagaggcgtttaaaccgcacaacctgccgatggtcttcaccgggactgccattctctatatcggttggtttggctttaacgccgggtcagcgggcacggcgaatgaaatcgcggcactggcatttgtgaatactgtggtcgcaacggcggcggcaattcttggctggatcttcggtgaatgggcgctgcgtggtaagccttcactgctgggggcgtgttctggcgcgattgccggtctggtcggcgtgacgccagcctgcggctacattggggttggcggcgcgttgattatcggcgtggtagctggtctggcgggcttgtggggcgttaccatgctcaaacgcttgctgcgggtggatgatccctgcgatgtcttcggtgtgcacggcgtttgtggcattgtcggctgtatcatgaccgggatttttgccgccagctcgctgggcggcgtgggcttcgctgaaggtgtgacgatgggccatcagttgctggtacagctggaaagcatcgccattacgatcgtctggtccggtgttgtggcatttatcggctacaaattggcggatctgacggttggtctgcgtgtaccggaagagcaggagcgagaagggctggatgtcaacagccacggcgagaatgcctataacgcgtaa

SEQ ID NO: 134 - DNA, 1401 bp, Escherichia coli
  misc_feature 1..1401: note = GABA permease GabP Escherichia coli
  source 1..1401: mol_type = genomic DNA
atggggcaatcatcgcaaccacatgagttaggcggcgggctgaagtcacgccacgtcaccatgttgtctattgccggtgttatcggcgcaagtctgtttgtcggttccagcgtcgccatcgccgaagcgggcccggcggtattactggcctatctgttcgccggattactggtggttatgattatgcggatgttggcggaaatggcagttgccacgcccgataccggttcgttttccacctatgccgataaagccattggccgctgggcgggttataccatcggctggctgtactggtggttttgggtactggttatcccgctggaagccaacatcgccgctatgatcctgcactcgtgggttccaggcattcccatctggttattttccctcgtcattaccctcgccttaactggcagtaatttattaagcgttaaaaactacggcgaatttgagttctggctggcgctgtgcaaagtcatcgctatcctggcctttattttccttggtgcagtcgcaattagcggtttttacccttatgccgaagtgagcgggatctcaagattgtgggatagcggcggctttatgcccaacggtttcggtgcggtattaagcgcgatgttgatcaccatgttctcgtttatgggcgcagaaattgtcaccattgccgccgcggaatccgacacgccggaaaaacatattgtccgcgccactaactcggttatctggcgtatttctatcttctatttgtgctctatttttgtcgtagtggcgttaataccgtggaatatgccggggctgaaagccgttggttcttatcgctcggttctggaattgctcaatattccccatgcgaaattaatcatggactgcgtgatattactttccgtaaccagctgtctgaactcggcgctgtataccgcgtcaaggatgctctactccttaagccgtcgcggtgatgcgcccgcggtaatgggcaaaatcaaccgcagtaaaaccccgtatgtggcggtgttactctccaccggagcggcatttttaacggtggtggtgaactattacgcacctgcgaaagtgtttaaattcctgatagacagctccggtgctatcgccctgctggtttatttagtcatcgccgtttcacagttgcggatgcgtaaaattctgcgagcagaaggaagcgaaattcgcttgcgcatgtggctttacccgtggctcacctggctggtaataggctttattacctttgtgttggtagtgatgctattccgcccggcgcaacagttagaagtgatctctaccggcttattagcgatagggattatctgtaccgtgccgattatggcgcgctggaaaaagctggtattgtggcaaaaaacacccgttcataatacgcgctga

SEQ ID NO: 135 - DNA, 1239 bp, Escherichia coli
  misc_feature 1..1239: note = mtnH Escherichia coli
  source 1..1239: mol_type = genomic DNA
atgacgaactatcgcgttgagagtagcagcggacgggcggcgcgcaagatgaggctcgcattaatgggacctgcgttcattgcggcgattggttatatcgatcccggtaactttgcgaccaatattcaggcgggtgccagcttcggctatcagctactgtgggttgtcgtttgggccaacctgatggcgatgctgattcagatcctctctgccaaactagggattgccaccggtaaaaatctggcggagcagattcgcgatcactatccgcgtcccgtagtgtggttctattgggttcaggcagaaattattgcgatggcaaccgacctggcggaatttattggtgcggcgatcggttttaaactcattcttggtgtctcgttgttgcagggcgcggtgctgacggggatcgcgactttcctgattttaatgctgcaacgtcgcgggcaaaaaccgctggagaaagtgattggcgggttactgttgtttgttgccgcggcttacattgtcgagttgattttctcccagcctaacctggcgcagctgggtaaaggaatggtgatcccgagtttacctacttcggaggcggtcttcctggcagcaggcgtgttaggggcgacgattatgccgcatgtgatttatttgcactcctcgctcactcagcatttacatggcggttcgcgtcaacaacgttattccgccaccaaatgggatgtggctatcgccatgacgattgccggttttgtcaatctggcgatgatggctacagctgcggcggcgttccacttttctggtcatactggtgttgccgatcttgatgaggcttatctgacgctgcaaccgctgttaagccatgctgcggcaacggtctttgggttaagtctggttgctgccggactgtcctcaacggtggtggggacactggcggggcaggtggtgatgcagggattcattcgcttccatatcccgctgtgggtgcgtcgtacagtcaccatgttgccgtcatttattgtcattctgatgggattagatccgacacggattctggttatgagtcaggtgctgttaagttttggtatcgccctggcgctggttccactgctgattttcaccagtgacagcaagttgatgggcgatctggtgaacagcaaacgcgtaaaacagacaggctgggtgattgtagtgctggtcgtggcgctgaatatctggttgttggtggggacggcgctgggattgtag

SEQ ID NO: 136 - protein, 287 aa, Salmonella enterica
  REGION 1..287: note = misc_feature - Tsx - Salmonella enterica subsp. enterica serovar Typhimurium LT2 (STM0413)
MKKTLLAVSAALALTSSFTANAAENDQPQYLSDWWHQSVNVVGSYHTRFSPKLNNDVYLEYEAFAKKDWFDFYGYIDIPKTFDWGNGNDKGIWSDGSPLFMEIEPRFSIDKLTGADLSFGPFKEWYFANNYIYDMGDNKASRQSTWYMGLGTDIDTGLPMGLSLNVYAKYQWQNYGASNENEWDGYRFKVKYFVPITDLWGGKLSYIGFTNFDWGSDLGDDPNRTSNSIASSHILALNYDHWHYSVVARYFHNGGQWQNGAKLNWGDGDFSAKSTGWGGYLVVGYNF

SEQ ID NO: 137 - protein, 406 aa, Bacillus halodurans
  REGION 1..406: note = misc_feature - BH1446 - Bacillus halodurans (BAB05165)
MNILWGLLGIVVVFLIAFAFSTNRRAIKPRTILGGLAIQLLFAIIVLKIPAGQALLESLTNVVLNIISYANEGIDFVFGGFFEEGSGVGFVFAINVLSVVIFFSALISILYYLGIMQFVIKIIGGALSWLLGTSKAESMSAAANIFVGQTEAPLVVKPYLPKMTQSELFAVMTGGLASVAGSVLIGYSLLGVPLQYLLAASFMAAPAGLIMAKMIMPETEKTTDAEDDFKLAKDEESTNLIDAAANGASTGLMLVLNIAAMLLAFVALIALINGILGWIGGLFGASQLSLELILGYVFAPLAFVIGIPWAEALQAGSYIGQKLVVNEFVAYLSFAPEIENLSDKAVMVISFALCGFANFSSLGILLGGLGKLAPSRRPDIARLGLRAILAGTLASLLSASIAGMLF

SEQ ID NO: 138 - protein, 393 aa, Bacillus subtilis
  REGION 1..393: note = misc_feature - nupC Bacillus subtilis subsp. subtilis 168 (BSU39410; CAA57663)
MKYLIGIIGLIVFLGLAWIASSGKKRIKIRPIVVMLILQFILGYILLNTGIGNFLVGGFAKGFGYLLEYAAEGINFVFGGLVNADQTTFFMNVLLPIVFISALIGILQKWKVLPFIIRYIGLALSKVNGMGRLESYNAVASAILGQSEVFISLKKELGLLNQQRLYTLCASAMSTVSMSIVGAYMTMLKPEYVVTALVLNLFGGFIIASIINPYEVAKEEDMLRVEEEEKQSFFEVLGEYILDGFKVAVVVAAMLIGFVAIIALINGIFNAVFGISFQGILGYVFAPFAFLVGIPWNEAVNAGSIMATKMVSNEFVAMTSLTQNGFHFSGRTTAIVSVFLVSFANFSSIGIIAGAVKGLNEKQGNVVARFGLKLLYGATLVSFLSAAIVGLIY

SEQ ID NO: 139 - protein, 404 aa, Bacillus subtilis
  REGION 1..404: note = misc_feature - yutK - Bacillus subtilis subsp. subtilis 168: BSU32180
MNVLWGLLGAVAIIAIAFLFSEKKSNIKIRTVIVGLCTQVAFGYIVLKWEAGRAVFLWFSSRVQLLIDYANEGISFIFGPLLKVGDSPAFALSVLPVIIFFSALIAVLYHLKIMQLVFRVIGGGLSKLLGTSKTESLAAAANIFVGQSESPLVIKPLIAGLTRSELFTIMTSGLSAVAGSTLFGYALLGIPIEYLLAASFMAAPAGLVFGKLIIPETEKTQTVKSDFKMDEGEGAANVIDAAAKGASTGLQIALNVGAMLLAFVALIAVVNGILGGAFGLFGLKGVTLESILGYVFSPIAFLIGVPWHEALQAGSYIGQKLVLNEFVAYSNFGSHIGEFSKKTATIISFALCGFANFSSIAIMLGTLGGLAPSRRSDIARLGLKAVLAGTLANLLSAAIAGMFI

SEQ ID NO: 140 - protein, 397 aa, Bacillus subtilis
  REGION 1..397: note = misc_feature - yxjA - Bacillus subtilis subsp. spizizenii W23 (BSUW23_19355)
MYFLLNLVGLIVIMAVVFLCSPQKKKIQWRPIITLIVLELLITWFMLGTKVGSWAIGKIGDFFTWLIACASDGIAFAFPSVMANETVDFFFSALLPIIFIVTFFDILTYFGILPWLIDKIGWVISKASRLPKLESFFSIQMMFLGNTEALAVIRQQLTVLNNNRLLTFGLMSMSSISGSIIGSYLSMVPATYVFTAIPLNCLNALIIANLLNPVHVPEDEDIIYTPPKEEKKDFFSTISNSMLVGMNMVIVILAMVIGYVALTSAVNGILGVFVHGLTIQTIFAYLFSPFAFLLGLPVHDAMYVAQLMGMKLATNEFVAMLDLKNNLTTLPPHTVAVATTFLTSFANFSTVGMIYGTYNSILDGEKSTVIGKNVWKLLVSGIAVSLLSAAIVGLFVW

SEQ ID NO: 141 - protein, 426 aa, Caulobacter crescentus
  REGION 1..426: note = misc_feature - ccCNT (CC2089) - Caulobacter crescentus CB15 (AAK24060)
MFRPENVQALAGLALTLGLCWLVSENRKRFPWGLAIGAVVIQVLLVLVLFGLPQAQQMLRGVNGAVEGLAASTQAGTAFVFGFLAGGDQPYPVSNPGAGFIFAFRVLPVILVVCALSALLWHWKILKWLAQGFGFVFQKTLGLRGPPALATAATIFMGQVEGPIFIRAYLDKLSRSELFMLIAVGMACVSGSTMVAYATILADVLPNAAAHVLTASIISAPAGVLLARIIVPSDPMEKSADLDLSTEDKTYGSSIDAVMKGTTDGLQIALNVGATLIVFVALATMVDKVLGAFPPVGGEPLSIARGLGVVFAPLAWSMGIPWKEAGTAGGLLGVKLILTEFTAFIQLSKVGEALLDERTRMIMTYALCGFANIGSVGMNVAGFSVLVPQRRQEVLGLVWKAMMAGFLATCLTASLVGLMPRSLFGL

SEQ ID NO: 142 - protein, 416 aa, Escherichia coli
  REGION 1..416: note = misc_feature - yeiJ - Escherichia coli K-12 W3110 (AAC75222; JW2148)
MDVMRSVLGMVVLLTIAFLLSVNKKKISLRTVGAALVLQVVIGGIMLWLPPGRWVAEKVAFGVHKVMAYSDAGSAFIFGSLVGPKMDTLFDGAGFIFGFRVLPAIIFVTALVSILYYIGVMGILIRILGGIFQKALNISKIESFVAVTTIFLGQNEIPAIVKPFIDRLNRNELFTAICSGMASIAGSTMIGYAALGVPVEYLLAASLMAIPGGILFARLLSPATESSQVSFNNLSFTETPPKSIIEAAATGAMTGLKIAAGVATVVMAFVAIIALINGIIGGVGGWFGFEHASLESILGYLLAPLAWVMGVDWSDANLAGSLIGQKLAINEFVAYLNFSPYLQTAGTLDAKTVAIISFALCGFANFGSIGVVVGAFSAVAPHRAPEIAQLGLRALAAATLSNLMSATIAGFFIGLA

SEQ ID NO: 143 - protein, 416 aa, Escherichia coli
  REGION 1..416: note = misc_feature - yeiM - Escherichia coli K-12 W3110 (AAC75225; JW2151)
MDIMRSVVGMVVLLAIAFLLSVNKKSISLRTVGAALLLQIAIGGIMLYFPPGKWAVEQAALGVHKVMSYSDAGSAFIFGSLVGPKMDVLFDGAGFIFAFRVLPAIIFVTALISLLYYIGVMGLLIRILGSIFQKALNISKIESFVAVTTIFLGQNEIPAIVKPFIDRMNRNELFTAICSGMASIAGSMMIGYAGMGVPIDYLLAASLMAIPGGILFARILSPATEPSQVTFENLSFSETPPKSFIEAAASGAMTGLKIAAGVATVVMAFVAIIALINGIIGGIGGWFGFANASLESIFGYVLAPLAWIMGVDWSDANLAGSLIGQKLAINEFVAYLSFSPYLQTGGTLEVKTIAIISFALCGFANFGSIGVVVGAFSAISPKRAPEIAQLGLRALAAATLSNLMSATIAGFFIGLA

SEQ ID NO: 144 - protein, 417 aa, Haemophilus influenzae
  REGION 1..417: note = misc_feature - HI0519 - Haemophilus influenzae Rd KW20 serotype d(AAC22177)
MSVLSSILGMVVLIAIAVLLSNNRKAISIRTVVGALAIQVGFAALILYVPAGKQALGAAADMVSNVIAYGNDGINFVFGGLADPSKPSGFIFAVKVLPIIVFFSGLISVLYYLGIMQVVIKVLGGALQKALGTSKAESMSAAANIFVGQTEAPLVVRPYIKNMTQSELFAIMVGGTASIAGSVMAGYAGMGVPLTYLIAASFMAAPAGLLFAKLMFPQTEQFTDKQPEDNDSEKPTNVLEAMAGGASAGMQLALNVGAMLIAFVGLIALINGILSGVGGWFGYGDLTLQSIFGLIFKPLAYLIGVTDGAEAGIAGQMIGMKLAVNEFVGYLEFAKYLQPDSAIVLTEKTKAIITFALCGFANFSSIAILIGGLGGMAPSRRSDVARLGIKAVIAGTLANLMSATIAGLFIGLGAAAL

SEQ ID NO: 145 - protein, 418 aa, Helicobacter pylori
  REGION 1..418: note = misc_feature - nupC (HP1180) - Helicobacter pylori 26695 (AAD08224)
MIFSSLFSVVGMAVLFLIAWVFSSNKRAINYRTIVSAFVIQVALGALALYVPLGREMLQGLASGIQSVISYGYEGVRFLFGNLAPNAKGDQGIGGFVFAINVLAIIIFFASLISLLYYLKIMPLFINLIGGALQKCLGTSRAESMSAAANIFVAHTEAPLVIKPYLKSMSDSEIFAVMCVGMASVAGPVLAGYASMGIPLPYLIAASFMSAPGGLLFAKIIYPQNETISSHADVSIEKHVNAIEAIANGASTGLNLALHVGAMLLAFVGMLALINGLLGVVGGFLGMEHLSLGLILGTLLKPLAFMLGIPWSQAGIAGEIIGIKIALNEFVGYMQLLPYLGDNPPLILSEKTKAIITFALCGFANLSSVAMLIGGLGSLVPKKKDLIVRLALKAVLVGTLSNFMSATIAGLFIGLNAH

SEQ ID NO: 146 - protein, 409 aa, Staphylococcus aureus
  REGION 1..409: note = misc_feature - nupC (SA0600) - Staphylococcus aureus subsp. aureus N315 (BAB41833)
MFLLINIIGLIVFLGIAVLFSRDRKNIQWQSIGILVVLNLFLAWFFIYFDWGQKAVRGAANGIAWVVQSAHAGTGFAFASLTNVKMMDMAVAALFPILLIVPLFDILMYFNILPKIIGGIGWLLAKVTRQPKFESFFGIEMMFLGNTEALAVSSEQLKRMNEMRVLTIAMMSMSSVSGAIVGAYVQMVPGELVLTAIPLNIVNAIIVSCLLNPVSVEEKEDIIYSLKNNEVERQPFFSFLGDSVLAAGKLVLIIIAFVISFVALADLFDRFINLITGLIAGWIGIKGSFGLNQILGVFMYPFALLLGLPYDEAWLVAQQMAKKIVTNEFVVMGEISKDIASYTPHHRAVITTFLISFANFSTIGMIIGTLKGIVDKKTSDFVSKYVPMMLLSGILVSLLTAAFVGLFAW

SEQ ID NO: 147 - protein, 409 aa, Staphylococcus aureus; identical to SEQ ID NO: 146
  REGION 1..409: note = misc_feature - nupC (SAV0645) -Staphylococcus aureus subsp. aureus Mu50 (BAB56807)

SEQ ID NO: 148 - protein, 400 aa, Streptococcus pyogenes
  REGION 1..400: note = misc_feature - nupC (SpNupC) - Streptococcus pyogenes SF370 serotype M1 (AAK34582)
MQFIYSIIGILLVLGIVYAISFNRKSVSLSLIGKALIVQFIIALILVRIPLGQQIVSVVSTGVTSVINCGQAGLNFVFGSLADSGAKTGFIFAIQTLGNIVFLSALVSLLYYVGILGFVVKWIGKGVGKIMKSSEVESFVAVANMFLGQTDSPILVSKYLGRMTDSEIMVVLVSGMGSMSVSILGGYIALGIPMEYLLIASTMVPIGSILIAKILLPQTEPVQKIDDIKMDNKGNNANVIDAIAEGASTGAQMAFSIGASLIAFVGLVSLINMMLSGLGIRLEQIFSYVFAPFGFLMGFDHKNILLEGNLLGSKLILNEFVSFQQLGHLIKSLDYRTALVATISLCGFANLSSLGICVSGIAVLCPEKRSTLARLVFRAMIGGIAVSMLSAFIVGIVTLF

SEQ ID NO: 149 - protein, 418 aa, Vibrio cholerae
  REGION 1..418: note = misc_feature - nupC (VC2352) - Vibrio cholerae O1 biovar El Tor N16961 (AAF95495)
MSLFMSLIGMAVLLGIAVLLSSNRKAINLRTVGGAFAIQFSLGAFILYVPWGQELLRGFSDAVSNVINYGNDGTSFLFGGLVSGKMFEVFGGGGFIFAFRVLPTLIFFSALISVLYYLGVMQWVIRILGGGLQKALGTSRAESMSAAANIFVGQTEAPLVVRPFVPKMTQSELFAVMCGGLASIAGGVLAGYASMGVKIEYLVAASFMAAPGGLLFAKLMMPETEKPQDNEDITLDGGDDKPANVIDAAAGGASAGLQLALNVGAMLIAFIGLIALINGMLGGIGGWFGMPELKLEMLLGWLFAPLAFLIGVPWNEATVAGEFIGLKTVANEFVAYSQFAPYLTEAAPVVLSEKTKAIISFALCGFANLSSIAILLGGLGSLAPKRRGDIARMGVKAVIAGTLSNLMAATIAGFFLSF

SEQ ID NO: 150 - protein, 405 aa, Vibrio cholerae
  REGION 1..405: note = misc_feature - nupC (VC1953) - Vibrio cholerae O1 biovar El Tor N16961 (AAF95101)
MGGVMSSLLGMGAILLVAWLFSTNRKNINLRTVSLALLLQIFFALLVLYVPAGKEALNRVTGAVSQLINYGQDGIGFVFGGLANGSVGFVFAINVLGIIIFFSALISGLYHLGIMPKVINLIGGGLQKLLGTGRAESLSATANIFVGMIEAPLVVKPYLHKMTDSQFFAVMTGGLASVAGGTLVGYASLGVELNYLIAAAFMSAPAGLLMAKIMLPETEHVDAAIAQDELDLPKSTNVVEAIADGAMSGVKIAVAVGATLLAFVSVIALLNGLLGWFGGWFGIELSFELIMGYVFAPVAWLIGIPWHEAITAGSLIGNKVVVNEFVAFIQLIEVKEQLSAHSQAIVTFALCGFANISTMAILIGGLGSLVPERRSFISQYGFRAIGAGVLANLMSASIAGVILSL

SEQ ID NO: 151 - protein, 425 aa, Escherichia coli
  REGION 1..425: note = misc_feature - yegT - Escherichia coli K-12 W3110 (P76417; JW2085)
MKTTAKLSFMMFVEWFIWGAWFVPLWLWLSKSGFSAGEIGWSYACTAIAAILSPILVGSITDRFFSAQKVLAVLMFAGALLMYFAAQQTTFAGFFPLLLAYSLTYMPTIALTNSIAFANVPDVERDFPRIRVMGTIGWIASGLACGFLPQILGYADISPTNIPLLITAGSSALLGVFAFFLPDTPPKSTGKMDIKVMLGLDALILLRDKNFLVFFFCSFLFAMPLAFYYIFANGYLTEVGMKNATGWMTLGQFSEIFFMLALPFFTKRFGIKKVLLLGLVTAAIRYGFFIYGSADEYFTYALLFLGILLHGVSYDFYYVTAYIYVDKKAPVHMRTAAQGLITLCCQGFGSLLGYRLGGVMMEKMFAYQEPVNGLTFNWSGMWTFGAVMIAIIAVLFMIFFRESDNEITAIKVDDRDIALTQGEVK

SEQ ID NO: 152 - protein, 418 aa, Escherichia coli
  REGION 1..418: note = misc_feature - nupG - Escherichia coli K-12 W3110 (P09452; JW2932
MNLKLQLKILSFLQFCLWGSWLTTLGSYMFVTLKFDGASIGAVYSSLGIAAVFMPALLGIVADKWLSAKWVYAICHTIGAITLFMAAQVTTPEAMFLVILINSFAYMPTLGLINTISYYRLQNAGMDIVTDFPPIRIWGTIGFIMAMWVVSLSGFELSHMQLYIGAALSAILVLFTLTLPHIPVAKQQANQSWTTLLGLDAFALFKNKRMAIFFIFSMLLGAELQITNMFGNTFLHSFDKDPMFASSFIVQHASIIMSISQISETLFILTIPFFLSRYGIKNVMMISIVAWILRFALFAYGDPTPFGTVLLVLSMIVYGCAFDFFNISGSVFVEKEVSPAIRASAQGMFLMMTNGFGCILGGIVSGKVVEMYTQNGITDWQTVWLIFAGYSVVLAFAFMAMFKYKHVRVPTGTQTVSH

SEQ ID NO: 153 - protein, 418 aa, Escherichia coli
  REGION 1..418: note = misc_feature - xapB - Escherichia coli K-12 W3110 (P45562; JW2397)
MSIAMRLKVMSFLQYFIWGSWLVTLGSYMINTLHFTGANVGMVYSSKGIAAIIMPGIMGIIADKWLRAERAYMLCHLVCAGVLFYAASVTDPDMMFWVMLVNAMAFMPTIALSNSVSYSCLAQAGLDPVTAFPPIRVFGTVGFIVAMWAVSLLHLELSSLQLYIASGASLLLSAYALTLPKIPVAEKKATTSLASKLGLDAFVLFKNPRMAIFFLFAMMLGAVLQITNVFGNPFLHDFARNPEFADSFVVKYPSILLSVSQMAEVGFILTIPFFLKRFGIKTVMLMSMVAWTLRFGFFAYGDPSTTGFILLLLSMIVYGCAFDFFNISGSVFVEQEVDSSIRASAQGLFMTMVNGVGAWVGSILSGMAVDYFSVDGVKDWQTIWLVFAGYALFLAVIFFFGFKYNHDPEKIKHRAVTH

SEQ ID NO: 154 - protein, 413 aa, Escherichia coli
  REGION 1..413: note = misc_feature - CC1628 - Caulobacter crescentus CB15 (AAK23606)
MGTSFRLFVMMVLQLAIWGAWAPKIFPYMGMLGFAPWQQSLVGSAWGVAALVGIFFSNQFADRNFSAERFLAVSHLIGGVALLGTAFSTEFWPFFACYLVFSLVYVPTLSVTNSIAFANLRDPAAGFGGVRMGGTVGWVLVSWPFVFLLGAQATVEQVRWIFLVAAIVSFVFAGYALTLPHTPPRKADDAVDKLAWRRAFKLLGAPFVFVLFVVTFIDSVIHNGYFVMADAFLTNRVGIAGNLSMVVLSLGQVAEIITMLLLGRVLAKLGWKVTMIVGVLGHAARFAVFAYFADSVPVIVAVQLLHGVCYAFFFATVYIFVDAVFPKDVRSSAQGLFNLLILGVGNVAASFIFPALIGRLTTDGSVDYTTLFLVPTAMALAAVCLLALFFRPPTRGPVSEADSASSAASSAQA

SEQ ID NO: 155 - protein, 419 aa, Escherichia coli
  REGION 1..419: note = misc_feature - codB - Escherichia coli K-12 W3110 (P25525; JW0327)
MSQDNNFSQGPVPQSARKGVLALTFVMLGLTFFSASMWTGGTLGTGLSYHDFFLAVLIGNLLLGIYTSFLGYIGAKTGLTTHLLARFSFGVKGSWLPSLLLGGTQVGWFGVGVAMFAIPVGKATGLDINLLIAVSGLLMTVTVFFGISALTVLSVIAVPAIACLGGYSVWLAVNGMGGLDALKAVVPAQPLDFNVALALVVGSFISAGTLTADFVRFGRNAKLAVLVAMVAFFLGNSLMFIFGAAGAAALGMADISDVMIAQGLLLPAIVVLGLNIWTTNDNALYASGLGFANITGMSSKTLSVINGIIGTVCALWLYNNFVGWLTFLSAAIPPVGGVIIADYLMNRRRYEHFATTRMMSVNWVAILAVALGIAAGHWLPGIVPVNAVLGGALSYLILNPILNRKTTAAMTHVEANSVE

SEQ ID NO: 156 - protein, 428 aa, Escherichia coli
  REGION 1..428: note = misc_feature - amtB - Escherichia coli K-12 MG1655 (B0451; 945084)
MKIATIKTGLASLAMLPGLVMAAPAVADKADNAFMMICTALVLFMTIPGIALFYGGLIRGKNVLSMLTQVTVTFALVCILWVVYGYSLAFGEGNNFFGNINWLMLKNIELTAVMGSIYQYIHVAFQGSFACITVGLIVGALAERIRFSAVLIFVVVWLTLSYIPIAHMVWGGGLLASHGALDFAGGTVVHINAAIAGLVGAYLIGKRVGFGKEAFKPHNLPMVFTGTAILYIGWFGFNAGSAGTANEIAALAFVNTVVATAAAILGWIFGEWALRGKPSLLGACSGAIAGLVGVTPACGYIGVGGALIIGVVAGLAGLWGVTMLKRLLRVDDPCDVFGVHGVCGIVGCIMTGIFAASSLGGVGFAEGVTMGHQLLVQLESIAITIVWSGVVAFIGYKLADLTVGLRVPEEQEREGLDVNSHGENAYNA

SEQ ID NO: 157 - protein, 466 aa, Escherichia coli
  REGION 1..466: note = misc_feature - GABA permease GabP Escherichia coli
MGQSSQPHELGGGLKSRHVTMLSIAGVIGASLFVGSSVAIAEAGPAVLLAYLFAGLLVVMIMRMLAEMAVATPDTGSFSTYADKAIGRWAGYTIGWLYWWFWVLVIPLEANIAAMILHSWVPGIPIWLFSLVITLALTGSNLLSVKNYGEFEFWLALCKVIAILAFIFLGAVAISGFYPYAEVSGISRLWDSGGFMPNGFGAVLSAMLITMFSFMGAEIVTIAAAESDTPEKHIVRATNSVIWRISIFYLCSIFVVVALIPWNMPGLKAVGSYRSVLELLNIPHAKLIMDCVILLSVTSCLNSALYTASRMLYSLSRRGDAPAVMGKINRSKTPYVAVLLSTGAAFLTVVVNYYAPAKVFKFLIDSSGAIALLVYLVIAVSQLRMRKILRAEGSEIRLRMWLYPWLTWLVIGFITFVLVVMLFRPAQQLEVISTGLLAIGIICTVPIMARWKKLVLWQKTPVHNTR

SEQ ID NO: 158 - protein, 412 aa, Escherichia coli
  REGION 1..412: note = misc_feature - mtnH Escherichia coli
MTNYRVESSSGRAARKMRLALMGPAFIAAIGYIDPGNFATNIQAGASFGYQLLWVVVWANLMAMLIQILSAKLGIATGKNLAEQIRDHYPRPVVWFYWVQAEIIAMATDLAEFIGAAIGFKLILGVSLLQGAVLTGIATFLILMLQRRGQKPLEKVIGGLLLFVAAAYIVELIFSQPNLAQLGKGMVIPSLPTSEAVFLAAGVLGATIMPHVIYLHSSLTQHLHGGSRQQRYSATKWDVAIAMTIAGFVNLAMMATAAAAFHFSGHTGVADLDEAYLTLQPLLSHAAATVFGLSLVAAGLSSTVVGTLAGQVVMQGFIRFHIPLWVRRTVTMLPSFIVILMGLDPTRILVMSQVLLSFGIALALVPLLIFTSDSKLMGDLVNSKRVKQTGWVIVVLVVALNIWLLVGTALGL

SEQ ID NO: 159 - DNA, 1320 bp, Escherichia coli
  misc_feature 1..1320: note = Nucleotide sequence: BCAA transporter BrnQ from E. coli
  source 1..1320: mol_type = genomic DNA
atgacccatcaattaagatcgcgcgatatcatcgctctgggctttatgacatttgcgttgttcgtcggcgcaggtaacattattttccctccaatggtcggcttgcaggcaggcgaacacgtctggactgcggcattcggcttcctcattactgccgttggcctaccggtattaacggtagtggcgctggcaaaagttggcggcggtgttgacagtctcagcacgccaattggtaaagtcgctggcgtactgctggcaacagtttgttacctggcggtggggccgctttttgctacgccgcgtacagctaccgtttcttttgaagtgggcattgcgccgctgacgggtgattccgcgctgccgctgtttatttacagcctggtctatttcgctatcgttattctggtttcgctctatccgggcaagctgctggataccgtgggcaacttccttgcgccgctgaaaattatcgcgctggtcatcctgtctgttgccgcaattatctggccggcgggttctatcagtacggcgactgaggcttatcaaaacgctgcgttttctaacggcttcgtcaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctgggttcagacagcgcgtcgctggtcgatcagtctgcaaacggtgcggcgatcctgcatgcttacgttcagcatacctttggcggcggcggtagcttcctgctggcggcgttaatcttcatcgcctgcctggtcacggcggttggcctgacctgtgcttgtgcagaattcttcgcccagtacgtaccgctctcttatcgtacgctggtgtttatcctcggcggcttctcgatggtggtgtctaacctcggcttgagccagctgattcagatctctgtaccggtgctgaccgccatttatccgccgtgtatcgcactggttgtattaagttttacacgctcatggtggcataattcgtcccgcgtgattgctccgccgatgtttatcagcctgctttttggtattctcgacgggatcaaggcatctgcattcagcgatatcttaccgtcctgggcgcagcgtttaccgctggccgaacaaggtctggcgtggttaatgccaacagtggtgatggtggttctggccattatctgggatcgtgcggcaggtcgtcaggtgacctccagcgctcactaa

SEQ ID NO: 160 - protein, 439 aa, Escherichia coli
  REGION 1..439: note = misc_feature - AA sequence: BCAA transporter BrnQ from E. coli
MTHQLRSRDIIALGFMTFALFVGAGNIIFPPMVGLQAGEHVWTAAFGFLITAVGLPVLTVVALAKVGGGVDSLSTPIGKVAGVLLATVCYLAVGPLFATPRTATVSFEVGIAPLTGDSALPLFIYSLVYFAIVILVSLYPGKLLDTVGNFLAPLKIIALVILSVAAIIWPAGSISTATEAYQNAAFSNGFVNGYLTMDTLGAMVFGIVIVNAARSRGVTEARLLTRYTVWAGLMAGVGLTLLYLALFRLGSDSASLVDQSANGAAILHAYVQHTFGGGGSFLLAALIFIACLVTAVGLTCACAEFFAQYVPLSYRTLVFILGGFSMVVSNLGLSQLIQISVPVLTAIYPPCIALVVLSFTRSWWHNSSRVIAPPMFISLLFGILDGIKASAFSDILPSWAQRLPLAEQGLAWLMPTVVMVVLAIIWDRAAGRQVTSSAH

SEQ ID NO: 161 - DNA, 18 bp, synthetic construct
  misc_feature 1..18: note = Synthetic: consensus sequence
  source 1..18: mol_type = other DNA
ttgttgayryrtcaacwa

SEQ ID NO: 162 - DNA, 15 bp, synthetic construct
  misc_feature 1..15: note = Synthetic: consensus sequence
  source 1..15: mol_type = other DNA
ttataatnattataa

SEQ ID NO: 163 - DNA, 355 bp, synthetic construct
  misc_feature 1..355: note = Synthetic: katG Regulatory sequence
  source 1..355: mol_type = other DNA
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 164 - DNA, 228 bp, synthetic construct
  misc_feature 1..228: note = Synthetic: dps Regulatory sequence
  source 1..228: mol_type = other DNA
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 165 - DNA, 334 bp, synthetic construct
  misc_feature 1..334: note = Synthetic: ahpC Regulatory sequence
  source 1..334: mol_type = other DNA
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 166 - DNA, 134 bp, synthetic construct
  misc_feature 1..134: note = Synthetic: oxyS Regulatory sequence
  source 1..134: mol_type = other DNA
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 167 - protein, 416 aa, Pseudomonas sp.
  REGION 1..416: note = misc_feature - Pseudomonas kynureninase
MTTRNDCLALDAQDSLAPLRQQFALPEGVIYLDGNSLGARPVAALARAQAVIAEEWGNGLIRSWNSAGWRDLSERLGNRLATLIGARDGEVVVTDTTSINLFKVLSAALRVQATRSPERRVIVTETSNFPTDLYIAEGLADMLQQGYTLRLVDSPEELPQAIDQDTAVVMLTHVNYKTGYMHDMQALTALSHECGALAIWDLAHSAGAVPVDLHQAGADYAIGCTYKYLNGGPGSQAFVWVSPQLCDLVPQPLSGWFGHSRQFAMEPRYEPSNGIARYLCGTQPITSLAMVECGLDVFAQTDMASLRRKSLALTDLFIELVEQRCAAHELTLVTPREHAKRGSHVSFEHPEGYAVIQALIDRGVIGDYREPRIMRFGFTPLYTTFTEVWDAVQILGEILDRKTWAQAQFQVRHSVT

SEQ ID NO: 168 - protein, 465 aa, Homo sapiens
  REGION 1..465: note = misc_feature - Human kynureninase
MEPSSLELPADTVQRIAAELKCHPTDERVALHLDEEDKLRHFRECFYIPKIQDLPPVDLSLVNKDENAIYFLGNSLGLQPKMVKTYLEEELDKWAKIAAYGHEVGKRPWITGDESIVGLMKDIVGANEKEIALMNALTVNLHLLMLSFFKPTPKRYKILLEAKAFPSDHYAIESQLQLHGLNIEESMRMIKPREGEETLRIEDILEVIEKEGDSIAVILFSGVHFYTGQHFNIPAITKAGQAKGCYVGFDLAHAVGNVELYLHDWGVDFACWCSYKYLNAGAGGIAGAFIHEKHAHTIKPALVGWFGHELSTRFKMDNKLQLIPGVCGFRISNPPILLVCSLHASLEIFKQATMKALRKKSVLLTGYLEYLIKHNYGKDKAATKKPVVNIITPSHVEERGCQLTITFSVPNKDVFQELEKRGVVCDKRNPNGIRVAPVPLYNSFHDVYKFTNLLTSILDSAETKN

SEQ ID NO: 169 - protein, 378 aa, Shewanella sp.
  REGION 1..378: note = misc_feature - Shewanella kynureninase
MLLNVKQDFCLAGPGYLLNHSVGRPLKSTEQALKQAFFAPWQESGREPWGQWLGVIDNFTAALASLFNGQPQDFCPQVNLSSALTKIVMSLDRLTRDLTRNGGAVVLMSEIDFPSMGFALKKALPASCELRFIPKSLDVTDPNVWDAHICDDVDLVFVSHAYSNTGQQAPLAQIISLARERGCLSLVDVAQSAGILPLDLAKLQPDFMIGSSVKWLCSGPGAAYLWVNPAILPECQPQDVGWFSHENPFEFDIHDFRYHPTALRFWGGTPSIAPYAIAAHSIEYFANIGSQVMREHNLQLMEPVVQALDNELVSPQEVDKRSGTIILQFGERQPQILAALAAANISVDTRSLGIRVSPHIYNDEADIARLLGVIKANR

SEQ ID NO: 170 - DNA, 1424 bp, Pseudomonas sp.
  misc_feature 1..1424: note = Ptet-kynU(Pseudomonas)
  source 1..1424: mol_type = genomic DNA
atctaatctagacatcattaattcctaatttttgttgacactctatcattgatagagttattttaccactccctatcagtgatagagaaaagtgaattatataaaagtgggaggtgcccgaatgacgacccgaaatgattgcctagcgttggatgcacaggacagtctggctccgctgcgccaacaatttgcgctgccggagggtgtgatatacctggatggcaattcgctgggcgcacgtccggtagctgcgctggctcgcgcgcaggctgtgatcgcagaagaatggggcaacgggttgatccgttcatggaactctgcgggctggcgtgatctgtctgaacgcctgggtaatcgcctggctaccctgattggtgcgcgcgatggggaagtagttgttactgataccacctcgattaatctgtttaaagtgctgtcagcggcgctgcgcgtgcaagctacccgtagcccggagcgccgtgttatcgtgactgagacctcgaatttcccgaccgacctgtatattgcggaagggttggcggatatgctgcaacaaggttacactctgcgtttggtggattcaccggaagagctgccacaggctatagatcaggacaccgcggtggtgatgctgacgcacgtaaattataaaaccggttatatgcacgacatgcaggctctgaccgcgttgagccacgagtgtggggctctggcgatttgggatctggcgcactctgctggcgctgtgccggtggacctgcaccaagcgggcgcggactatgcgattggctgcacgtacaaatacctgaatggcggcccgggttcgcaagcgtttgtttgggtttcgccgcaactgtgcgacctggtaccgcagccgctgtctggttggttcggccatagtcgccaattcgcgatggagccgcgctacgaaccttctaacggcattgctcgctatctgtgcggcactcagcctattactagcttggctatggtggagtgcggcctggatgtgtttgcgcagacggatatggcttcgctgcgccgtaaaagtctggcgctgactgatctgttcatcgagctggttgaacaacgctgcgctgcacacgaactgaccctggttactccacgtgaacacgcgaaacgcggctctcacgtgtcttttgaacaccccgagggttacgctgttattcaagctctgattgatcgtggcgtgatcggcgattaccgtgagccacgtattatgcgtttcggtttcactcctctgtatactacttttacggaagtttgggatgcagtacaaatcctgggcgaaatcctggatcgtaagacttgggcgcaggctcagtttcaggtgcgccactctgttacttaaaaataaaacgaaaggctcagtcgaaagactgggcctttcgttttatctgttg

SEQ ID NO: 171 - DNA, 1571 bp, Homo sapiens
  misc_feature 1..1571: note = Ptet-kynU(Human)
  source 1..1571: mol_type = genomic DNA
atctaatctagacatcattaattcctaatttttgttgacactctatcattgatagagttattttaccactccctatcagtgatagagaaaagtgaatatcaagacacgaggaggtaagattatggagccttcatctttagaactgccagcggacacggtgcagcgcatcgcggcggaactgaagtgccatccgactgatgagcgtgtggcgctgcatctggacgaagaagataaactgcgccactttcgtgaatgtttttatattcctaaaattcaagacttgccgccggtagatttgagtctcgttaacaaagatgaaaacgcgatctactttctgggcaactctctgggtctgcaaccaaaaatggttaaaacgtacctggaggaagaactggataaatgggcaaaaatcgcggcttatggtcacgaagtgggcaagcgtccttggattactggcgacgagtctattgtgggtttgatgaaagatattgtgggcgcgaatgaaaaggaaattgcactgatgaatgctctgaccgttaatctgcacctgctgatgctgtctttttttaaaccgaccccgaaacgctacaaaatactgctggaagcgaaagcgtttccgtcggatcactatgctatagaaagtcaactgcagttgcatggtctgaatatcgaggaatctatgcgcatgattaaaccgcgtgagggtgaagaaacgctgcgtattgaagacattctggaagttattgaaaaagaaggtgattctatcgcagttatactgttttctggcgtgcacttttatacaggtcagcacttcaatatcccggcaatcactaaagcggggcaggcaaaaggctgctatgttggttttgacctggcgcatgcagtggggaatgttgaactgtatctgcacgattggggcgttgatttcgcgtgttggtgtagctacaaatatctgaacgctggcgcgggtggcattgctggcgcttttattcacgaaaaacacgcgcacaccattaaaccggctctggttggctggttcggtcatgagctgagtactcgctttaaaatggataacaaactgcaattgattccgggtgtttgcggcttccgtatcagcaatccgccgattctgctggtttgcagcctgcacgctagtctggaaatctttaagcaggcgactatgaaagcgctgcgcaaaaaatctgtgctgctgaccggctatctggagtatctgatcaaacacaattatggcaaagataaagctgcaactaaaaaaccggtagtgaacattatcaccccctcacacgtggaggagcgcggttgtcagctgactattactttcagtgtacctaataaagatgtgttccaggaactggaaaaacgcggcgttgtttgtgataaacgtaacccgaatggtattcgcgtggctcctgtgccgctgtacaattcattccacgatgtttataaattcaccaacctgctgacttctattctcgacagtgctgagactaaaaattaaaaataaaacgaaaggctcagtcgaaagactgggcctttcgttttatctgttg

SEQ ID NO: 172 - DNA, 1310 bp, Shewanella sp.
  misc_feature 1..1310: note = ptet-kynU(Shewanella)
  source 1..1310: mol_type = genomic DNA
atctaatctagacatcattaattcctaatttttgttgacactctatcattgatagagttattttaccactccctatcagtgatagagaaaagtgaatggttcaccaccacaaggagggattatgctgctgaatgtaaaacaggacttttgcctggcaggcccgggctacctgctgaatcactcggttggccgtccgctgaaatcaactgagcaagcgctgaaacaagcattttttgctccgtggcaagagagcggtcgtgaaccgtggggccagtggctgggtgttattgataatttcactgctgcgctggcatctctgtttaatggtcaaccgcaggatttttgtccgcaggttaacctgagcagcgcgctgactaaaattgtgatgtcactggatcgtctgactcgcgatctgacccgcaatggcggtgctgttgtgctgatgtctgaaatcgatttcccatctatgggcttcgcgttgaaaaaagcgctgccagcgagctgcgaactgcgttttatcccgaaaagtctggacgtgactgatccgaacgtatgggatgcacacatctgtgatgatgtagacctggtttttgtgtctcacgcctatagtaatacgggccaacaggctccgctggcgcaaatcatctctctggcgcgtgaacgtggctgcctgtcactggtggatgtagcgcaatcagcggggattttgccgctggatctggcgaaactgcaaccggacttcatgatcggcagttcggttaaatggctgtgctcgggccctggtgcggcatatctgtgggttaatccggcgattctgccggaatgtcagccgcaggatgtgggctggttttcacatgagaatccctttgaattcgacatccacgatttccgctaccacccgactgcactgcgcttttggggtggtacgccgtcgatcgcgccttatgcgatcgcggcgcactcgatcgaatattttgccaatatcggctcgcaagtgatgcgtgaacacaacctgcaactgatggaaccggtggttcaggcgctggacaatgaactggtgagcccgcaggaagtggataaacgctcaggcactattattctgcaattcggtgaacgtcaaccgcaaattctggcggctctggctgcggcgaacatttcggtggacactcgttctttggggattcgtgttagtccgcacatttataatgatgaggcggacattgcgcgcctgctgggtgtgatcaaagcaaatcgctaaaaataaaacgaaaggctcagtcgaaagactgggcctttcgttttatctgttg

(19) United States
(12) Patent Application Publication
(10) Pub. No.: US 2024/0382536 A1
(43) Pub. Date: Nov. 21, 2024

(54) BACTERIA ENGINEERED TO TREAT A DISEASE OR DISORDER

(71) Applicant: Synlogic Operating Company, Inc., Cambridge, MA (US)

(72) Inventors: Dean Falb, Sherborn, MA (US); Paul F. Miller, Salem, CT (US); Jonathan W. Kotula, Berkeley, CA (US); Vincent M. Isabella, Medford, MA (US); Suman Machinani, Monroe, NY (US); Adam B. Fisher, Cambridge, MA (US); Yves Millet, Newton, MA (US)

(21) Appl. No.: 18/537,897

(22) Filed: Dec. 13, 2023

Related U.S. Application Data
(63) Continuation of application No. 17/124,661, filed on Dec. 17, 2020, now Pat. No. 11,883,439.
Application No. 17/124,661 is a continuation of application No. 15/852,762, filed on Dec. 22, 2017, now Pat. No. 10,933,102.
Application No. 15/852,762 is a continuation of application No. 15/319,564, filed on Dec. 16, 2016, now Pat. No. 9,889,164.
Application No. 15/319,564 is a continuation-in-part of application No. 15/154,934, filed on May 13, 2016, now Pat. No. 10,610,546.
Application No. 15/154,934 is a continuation-in-part of application No. PCT/US2016/032562, filed on May 13, 2016.
Application No. PCT/US2016/032562 is a continuation-in-part of application No. PCT/US2016/020530, filed on Mar. 2, 2016.
Application No. PCT/US2016/020530 is a continuation-in-part of application No. 14/960,333, filed on Dec. 4, 2015, now Pat. No. 9,487,764.
Application No. 14/960,333 is a continuation-in-part of application No. PCT/US2015/064140, filed on Dec. 4, 2015.
(60) Provisional application No. 62/336,338, filed on May 13, 2016.
(60) Provisional application No. 62/335,780, filed on May 13, 2016.
(60) Provisional application No. 62/335,940, filed on May 13, 2016.
(60) Provisional application No. 62/336,012, filed on May 13, 2016.
(60) Provisional application No. 62/314,322, filed on Mar. 28, 2016.
(60) Provisional application No. 62/313,691, filed on Mar. 25, 2016.
(60) Provisional application No. 62/293,749, filed on Feb. 10, 2016.
(60) Provisional application No. 62/277,654, filed on Jan. 12, 2016.
(60) Provisional application No. 62/277,413, filed on Jan. 11, 2016.
(60) Provisional application No. 62/277,346, filed on Jan. 11, 2016.
(60) Provisional application No. 62/263,329, filed on Dec. 4, 2015.
(60) Provisional application No. 62/256,041, filed on Nov. 16, 2015.
(60) Provisional application No. 62/256,039, filed on Nov. 16, 2015.
(60) Provisional application No. 62/256,052, filed on Nov. 16, 2015.
(60) Provisional application No. 62/212,223, filed on Aug. 31, 2015.
(60) Provisional application No. 62/199,445, filed on Jul. 31, 2015.
(60) Provisional application No. 62/183,935, filed on Jun. 24, 2015.
(60) Provisional application No. 62/173,761, filed on Jun. 10, 2015.
(60) Provisional application No. 62/173,706, filed on Jun. 10, 2015.
(60) Provisional application No. 62/173,710, filed on Jun. 10, 2015.
(60) Provisional application No. 62/161,137, filed on May 13, 2015.

(51) Int. Cl. A61K 35/74; A61K 35/00; A61K 35/741; A61K 38/00; A61K 39/02; C07K 14/195; C07K 14/245; C12N 9/06; C12N 9/88; C12N 15/70

(52) U.S. Cl. CPC A61K 35/74; A61K 35/741; A61K 39/02; C07K 14/195; C07K 14/245; C12N 9/0014; C12N 9/0022; C12N 9/88; C12N 15/70; C12Y 104/03002; C12Y 403/01024; A61K 2035/11; A61K 38/00; Y02A 50/30

(57) ABSTRACT

Genetically programmed microorganisms, such as bacteria, pharmaceutical compositions thereof, and methods of modulating and treating a disease and/or disorder are disclosed.

### RELATED APPLICATIONS

This application is a continuation of U.S. application Ser. No. 17/124,661, filed Dec. 17, 2020, which, in turn, is a continuation of U.S. application Ser. No. 15/852,762, filed on Dec. 22, 2017, and now issued as U.S. Pat. No. 10,933,102, which, in turn, is a continuation of U.S. application Ser. No. 15/319,564, filed on Dec. 16, 2016, now issued as U.S. Pat. No. 9,889,164 on Feb. 13, 2018, which is a 35 U.S.C. § 371 national stage filing of International Application No. PCT/US2016/032565, filed on May 13, 2016. PCT/US2016/032565, filed on May 13, 2016, in turn claims priority to U.S. Provisional Patent Application No. 62/335,780, filed on May 13, 2016; U.S. Provisional Patent Application No. 62/336,338, filed on May 13, 2016; U.S. Provisional Patent Application No. 62/336,012, filed on May 13, 2016; and U.S. Provisional Patent Application No. 62/335,940, filed on May 13, 2016. PCT/US2016/032565, filed on May 13, 2016, is further a continuation-in-part of U.S. patent application Ser. No. 15/154,934, filed on May 13, 2016; and a continuation-in-part of International Application No. PCT/US2016/032562, filed on May 13, 2016. PCT/US2016/032565, filed on May 13, 2016, further claims priority to U.S. Provisional Patent Application No. 62/277,654, filed on Jan. 12, 2016; U.S. Provisional Patent Application No. 62/277,413, filed on Jan. 11, 2016; and U.S. Provisional Patent Application No. 62/293,749, filed on Feb. 10, 2016. PCT/US2016/032565, filed on May 13, 2016, is further a continuation-in-part of International Application No. PCT/US2016/020530, filed on Mar. 2, 2016. PCT/US2016/032565, filed on May 13, 2016, further claims priority to U.S. Provisional Patent Application No. 62/173,761, filed on Jun. 10, 2015; U.S. Provisional Patent Application No. 62/173,706, filed on Jun. 10, 2015; U.S. Provisional Patent Application No. 62/173,710, filed on Jun. 10, 2015; U.S. Provisional Patent Application No. 62/277,346, filed on Jan. 11, 2016; U.S. Provisional Patent Application No. 62/199,445, filed on Jul. 31, 2015; U.S. Provisional Patent Application No. 62/314,322, filed on Mar. 28, 2016; and U.S. Provisional Patent Application No. 62/313,691, filed on Mar. 25, 2016. PCT/US2016/032565, filed on May 13, 2016, is further a continuation-in-part of U.S. patent application Ser. No. 14/960,333, filed on Dec. 4, 2015, now issued as U.S. Pat. No. 9,487,764 on Nov. 8, 2016; and a continuation-in-part of International Application No. PCT/US2015/064140, filed on Dec. 4, 2015. PCT/US2016/032565, filed on May 13, 2016, further claims priority to U.S. Provisional Patent Application No. 62/263,329, filed on Dec. 4, 2015; U.S. Provisional Patent Application No. 62/256,041, filed on Nov. 16, 2015; U.S. Provisional Patent Application No. 62/256,039, filed on Nov. 16, 2015; U.S. Provisional Patent Application No. 62/212,223, filed on Aug. 31, 2015; U.S. Provisional Patent Application No. 62/183,935, filed on Jun. 24, 2015; U.S. Provisional Patent Application No. 62/256,052, filed on Nov. 16, 2015; and U.S. Provisional Patent Application No. 62/161,137, filed on May 13, 2015. The entire contents of each of the foregoing applications are expressly incorporated herein by reference.

### SEQUENCE LISTING

The application contains a Sequence Listing which has been submitted electronically in .XML format and is hereby incorporated by reference in its entirety. Said. XML copy, created on Dec. 11, 2023, is named “126046-01405.xml” and is 402,506 bytes in size. The sequence listing contained in this .XML file is part of the specification and is hereby incorporated by reference herein in its entirety.

### BACKGROUND OF THE INVENTION

It has recently been discovered that the microbiome in mammals plays a large role in health and disease (see Cho and Blaser, Nature Rev. Genet., 13:260-270, 2012 and Owyang and Wu, Gastroenterol., 146 (6): 1433-1436, 2014). Indeed, bacteria-free animals have abnormal gut epithelial and immune function, suggesting that the microbiome in the gut plays a critical role in the mammalian immune system. Specifically, the gut microbiome has been shown to be involved in diseases, including, for example, immune diseases (such as Inflammatory Bowel Disease), autism, liver disease, cancer, food allergy, metabolic diseases (such as urea cycle disorder, phenylketonuria, and maple syrup urine disease), obesity, and infection, among many others.

Fecal transplantation of native microbial strains has recently garnered much attention for its potential to treat certain microbial infections and immune diseases in the gut (Owyang and Wu, 2014). There have also been recent efforts to engineer microbes to produce, e.g., secrete, therapeutic molecules and administer them to a subject in order to deliver the therapeutic molecule(s) directly to the site where therapy is needed, such as various sites in the gut. However, such efforts have been frustrated for several reasons, mostly relating to the constitutive production of the bacteria and its gene product(s). For example, the viability and stability of the engineered microbes have been compromised due, in part, to the constitutive production of large amounts of foreign protein(s). Unfortunately, genetically engineered microbes which have been engineered to express intracellular therapeutic enzymes which degrade target molecules associated with disease states or disorders, e.g., diseases or disorders associated with the overexpression of a molecule which is harmful to a subject, have also been shown to have low efficacy and enzyme activity levels in vitro and in vivo. Accordingly, a need exists for improved genetically engineered microbes which are useful for therapeutic purposes.

### SUMMARY

The instant invention surprisingly provides genetically engineered microbes which express a heterologous transporter in order to regulate, e.g., increase, the transport of target molecules associated with disease into the genetically engineered microbes in order to increase the therapeutic efficacy of the microbe.

In one aspect, the invention provides a genetically-engineered non-pathogenic microorganism comprising at least one heterologous gene encoding a substrate transporter, wherein the gene encoding the substrate transporter is operably linked to an inducible promoter.

In one embodiment, the bacterium is a Gram-positive bacterium. In one embodiment, the bacterium is a Gram-negative bacterium. In one embodiment, the bacterium is an obligate anaerobic bacterium. In one embodiment, the bacterium is a facultative anaerobic bacterium.

In one embodiment, the bacterium is an aerobic bacterium. In one embodiment, the bacterium is selected from Clostridium novyi NT, Clostridium butyricum, E. coli Nissle, and E. Coli K-12.

In one embodiment, the inducible promoter is induced by low-oxygen or anaerobic conditions. In one embodiment, the inducible promoter is selected from a FNR-inducible promoter, an ANR-inducible promoter, and a DNR-inducible promoter. In one embodiment, the inducible promoter is P-fnrs promoter.

In one embodiment, the substrate transporter is capable of importing into the bacterium a substrate selected from the group consisting of an amino acid, a nucleoside, kynurenine, prostaglandin E2, lactic acid, propionate, bile salt, γ-aminobutyric acid (GABA), manganese, a toxin, and a peptide.

In one embodiment, the substrate transporter is an amino acid transporter capable of importing into the bacterium an amino acid selected from the group consisting of leucine, isoleucine, valine, arginine, lysine, asparagine, serine, glycine, glutamine, tryptophan, methionine, threonine, cysteine, tyrosine, phenylalanine, glutamic acid, aspartic acid, alanine, histidine, and proline.

In one embodiment, the heterologous gene encoding the amino acid transporter is from Agrobacterium tumefaciens, Anabaena cylindrical, Anabaena variabilis, Bacillus amyloliquefaciens, Bacillus atrophaeus, Bacillus halodurans, Bacillus methanolicus, Bacillus subtilis, Caenorhabditis elegans, Clostridium botulinum, Corynebacterium glutamicum, Escherichia coli, Flavobacterium limosediminis, Helicobacter pylori, Klebsiella pneumonia, Lactococcus lactis, Lactobacillus saniviri, Legionella pneumophila Methylobacterium aquaticum, Mycobacterium bovis, Photorhabdus luminescens, Pseudomonas aeruginosa, Pseudomonas fluorescens, Saccharomyces cerevisiae, Salmonella enterica, Sinorhizobium meliloti, or Ustilago maydis. In one embodiment, the heterologous gene encoding the amino acid transporter has a sequence with at least 90% identity to any one of SEQ ID NOs: 9, 10, 13-18, 25, 26, 29, 35, 41-44, 46-48, 59-62, 69, 87, 91, 94-96, 98, or 103. In one embodiment, the heterologous gene encoding the amino acid transporter has a sequence comprising any one of SEQ ID NOs: 9, 10, 13-18, 25, 26, 29, 35, 41-44, 46-48, 59-62, 69, 87, 91, 94-96, 98, or 103. In one embodiment, the heterologous gene encoding the amino acid transporter has a sequence consisting of any one of SEQ ID NOs: 9, 10, 13-18, 25, 26, 29, 35, 41-44, 46-48, 59-62, 69, 87, 91, 94-96, 98, or 103.

In one embodiment, the substrate transporter is a nucleoside transporter capable of importing into the bacterium a nucleoside selected from the group consisting of adenosine, guanosine, uridine, inosine, xanthosine, thymidine and cytidine. In one embodiment, the heterologous gene encoding the nucleoside transporter is from Bacillus halodurans, Bacillus subtilis, Caulobacter crescentus, Escherichia coli, Haemophilus influenzae, Helicobacter pylori, Pseudomonas, Bacillus subtilis, Escherichia coli, Prevotella intermedia, Porphytomonas gingivalis, Salmonella typhimurium, Salmonella enterica, or Vibrio cholera.

In one embodiment, the heterologous gene encoding the nucleoside transporter has a sequence with at least 90% identity to any one of SEQ ID NOs: 108-128. In one embodiment, the heterologous gene encoding the amino acid transporter has a sequence comprising any one of SEQ ID NOs: 108-128. In one embodiment, the heterologous gene encoding the amino acid transporter has a sequence consisting of any one of SEQ ID NOs: 108-128.

In one embodiment, the substrate transporter is a kynurenine transporter capable of importing kynurenine into the bacterium. In one embodiment, the heterologous gene encoding the kynurenine transporter is from Escherichia coli, Saccharomyces cerevisiae or Corynebacterium glutamicum. In one embodiment, the heterologous gene encoding the kynurenine transporter has a sequence with at least 90% identity to any one of SEQ ID NOs: 46-48. In one embodiment, the heterologous gene encoding the kynurenine transporter has a sequence comprising any one of SEQ ID NOs: 46-48. In one embodiment, the heterologous gene encoding the amino acid transporter has a sequence consisting of any one of SEQ ID NOs: 46-48.

In one embodiment, the substrate transporter is a prostaglandin E2 transporter capable of importing prostaglandin E2 into the bacterium. In one embodiment, the heterologous gene encoding the prostaglandin E2 (PGE2) transporter is from Escherichia coli, Saccharomyces cerevisiae or Corynebacterium glutamicum.

In one embodiment, the substrate transporter is a lactic acid transporter capable of importing lactic acid into the bacterium. In one embodiment, the heterologous gene encoding the lactic acid transporter is from Escherichia coli, Saccharomyces cerevisiae and Corynebacterium glutamicum.

In one embodiment, the substrate transporter is a propionate transporter capable of importing propionate into the bacterium. In one embodiment, the heterologous gene encoding the propionate transporter is from Bacillus subtilis, Campylobacter jejuni, Clostridium perfringens, Escherichia coli, Lactobacillus delbrueckii, Mycobacterium smegmatis, Nocardia farcinica, Pseudomonas aeruginosa, Salmonella typhimurium, Virgibacillus, or Staphylococcus aureus. In one embodiment, the heterologous gene encoding the propionate transporter has a sequence with at least 90% identity to any one of SEQ ID NOs: 129-130. In one embodiment, the heterologous gene encoding the propionate transporter has a sequence comprising any one of SEQ ID NOs: 129-130. In one embodiment, the heterologous gene encoding the propionate transporter has a sequence consisting of any one of SEQ ID NOs: 129-130.

In one embodiment, the substrate transporter is a bile salt transporter capable of importing bile salt into the bacterium. In one embodiment, the heterologous gene encoding the bile salt transporter is from Lactobacillus johnsonni. In one embodiment, the heterologous gene encoding the bile salt acid transporter has a sequence with at least 90% identity to any one of SEQ ID NOs: 131-132. In one embodiment, the heterologous gene encoding the bile salt transporter has a sequence comprising any one of SEQ ID NOs: 131-132. In one embodiment, the heterologous gene encoding the bile salt transporter has a sequence consisting of any one of SEQ ID NOs: 131-132.

In one embodiment, the substrate transporter is a bile salt transporter capable of importing ammonia into the bacterium. In one embodiment, the heterologous gene encoding the ammonia transporter is from Corynebacterium glutamicum, Escherichia coli, Streptomyces coelicolor or Ruminococcus albus. In one embodiment, the heterologous gene encoding the ammonia transporter has a sequence with at least 90% identity to SEQ ID NO:133. In one embodiment, the heterologous gene encoding the ammonia transporter has a sequence comprising SEQ ID NO: 133. In one embodiment, the heterologous gene encoding the ammonia transporter has a sequence consisting of SEQ ID NO:133.

In one embodiment, the substrate transporter is a γ-aminobutyric acid (GABA) transporter capable of importing GABA into the bacterium. In one embodiment, the heterologous gene encoding the GABA transporter is from Escherichia coli or Bacillus subtilis. In one embodiment, the heterologous gene encoding the GABA transporter has a sequence with at least 90% identity to SEQ ID NO:134. In one embodiment, the heterologous gene encoding the GABA transporter has a sequence comprising SEQ ID NO:134. In one embodiment, the heterologous gene encoding the GABA transporter has a sequence consisting of SEQ ID NO: 134.

In one embodiment, the substrate transporter is a manganese transporter capable of importing manganese into the bacterium. In one embodiment, the heterologous gene encoding the manganese transporter is from Bacillus subtilis, Staphylococcus aureus, Salmonella typhimurium, Shigella flexneri, Yersinia pestis, or Escherichia coli. In one embodiment, the heterologous gene encoding the manganese transporter has a sequence with at least 90% identity to SEQ ID NO:135. In one embodiment, the heterologous gene encoding the manganese transporter has a sequence comprising SEQ ID NO:135. In one embodiment, the heterologous gene encoding the manganese transporter has a sequence consisting of SEQ ID NO: 135.

In one embodiment, the substrate transporter is a toxin transporter capable of importing a toxin into the bacterium. In one embodiment, the substrate transporter is a peptide transporter capable of importing a peptide into the bacterium.

In one embodiment, the heterologous gene encoding the substrate transporter and operatively linked promoter are present on a chromosome in the bacterium. In one embodiment, the heterologous gene encoding the substrate transporter and operatively linked promoter are present on a plasmid in the bacterium.

In one embodiment, the bacterium is an auxotroph comprising a deletion or mutation in a gene required for cell survival and/or growth. In one embodiment, the gene required for cell survival and/or growth is selected from thyA, dapD, and dapA.

In one embodiment, the bacterium comprises a kill switch.

In one aspect, the present disclosure provides a pharmaceutical composition comprising a recombinant bacterium disclosed herein and a pharmaceutically acceptable carrier.

In another aspect, the present invention provides a method of treating a disease in a subject in need thereof comprising the step of administering to the subject a pharmaceutical composition comprising a recombinant bacterium disclosed herein and a pharmaceutically acceptable carrier.

### BRIEF DESCRIPTION OF THE FIGURES

FIG. 1 depicts a synthetic biotic for treating phenylketonuria (PKU) and disorders characterized by hyperphenylalaninemia.

FIG. 2 depicts a synthetic biotic for treating phenylketonuria (PKU) and disorders characterized by hyperphenylalaninemia.

FIG. 3 is a graph showing that PheP and AroP Transport Phe at the same rate.

FIG. 4 depicts a schematic representation of the construction of a pheP knock-in strain, wherein recombineering is used to insert a 214 copy of pheP into the Nissle lacZ gene.

FIG. 5 depicts the gene organization of an exemplary construct comprising a gene encoding PheP, a gene coding TetR, and a Tet promoter sequence for chromosomal insertion e.g., as for example comprised in SYN-PKU203, SYN-PKU401, SYN-PKU402, SYN-PKU302, and SYN-PKU303.

FIGS. 6A and 6B depict the gene organization of an exemplary construct, comprising a cloned PAL3 gene under the control of an FNR promoter sequence, on a low-copy, kanamycin-resistant plasmid (pSC101 origin of replication, (FIG. 6A). Under anaerobic conditions, PAL3 degrades phenylalanine to non-toxic trans-cinnamate. FIG. 6B depicts an additional copy of the endogenous E. coli high affinity phenylalanine transporter, pheP, driven by the PfnrS promoter and inserted into the lacZ locus on the Nissle chromosome.

FIGS. 7A, 7B, and 7C depict schematic diagrams of non-limiting embodiments of the disclosure. FIG. 7A depicts phenylalanine degradation components integrated into the E. coli Nissle chromosome. In some embodiments, engineered plasmid-free bacterial strains are used to prevent plasmid conjugation in vivo. In some embodiments, multiple insertions of the PAL gene result in increased copy number and/or increased phenylalanine degradation activity. In some embodiments, a copy of the endogenous E. coli high affinity phenylalanine transporter, pheP, is driven by the PfnrS promoter and is inserted into the lacZ locus. FIG. 7B depicts a schematic diagram of one non-limiting embodiment of the disclosure, wherein the E. coli Nissle chromosome is engineered to contain four copies of PfnrS-PAL inserted at four different insertion sites across the genome (malE/K, yicS/nepI, agaI/rsmI, and cea), and one copy of a phenylalanine transporter gene inserted at a different insertion site (lacZ). In this embodiment, the PAL gene is PAL3 derived from P. luminescens, and the phenylalanine transporter gene is phcP derived from E. coli. In one embodiment, the strain is SYN-PKU511. FIG. 7C depicts a schematic diagram of one preferred embodiment of the disclosure, wherein the E. coli Nissle chromosome is engineered to contain five copies of PAL under the control of an oxygen level-dependent promoter (e.g., PfnrS-PAL3) inserted at different integration sites on the chromosome (malE/K, yicS/nepI, malP/T, agaI/rsmI, and cca), and one copy of a phenylalanine transporter gene under the control of an oxygen level-dependent promoter (e.g., PfnrS-pheP) inserted at a different integration site on the chromosome (lacZ). The genome is further engineered to include a thyA auxotrophy, in which the thyA gene is deleted and/or replaced with an unrelated gene, as well as a kanamycin resistance gene.

FIG. 8A depicts phenylalanine concentrations in samples comprising bacteria expressing PAL1 or PAL3 on low-copy (LC) or high-copy (HC) plasmids, or further comprising a copy of pheP driven by the Tet promoter integrated into the chromosome. Bacteria were induced with ATC, and then grown in culture medium supplemented with 4 mM (660,000 ng/mL) of phenylalanine to an OD600 of 2.0. Samples were removed at 0 hrs, 2 hrs, and 4 hrs post-induction and phenylalanine concentrations were determined by mass spectrometry. Notably, the additional copy of pheP permitted the degradation of phenylalanine (4 mM) in 4 hrs. FIG. 8B depicts cinnamate levels in samples at 2 hrs and 4 hrs post-induction. In some embodiments, cinnamate may be used as an alternative biomarker for strain activity. PheP overexpression improves phenylalanine metabolism in engineered bacteria. Strains analyzed in this data set are SYN-PKU101, SYN-PKU102, SYN-PKU202, SYN-PKU201, SYN-PKU401, SYN-PKU402, SYN-PKU203, SYN-PKU302, SYN-PKU303.

FIGS. 9A and 9B depict the state of one non-limiting embodiment of the PAL construct under non-inducing (FIG. 9A) and inducing (FIG. 9B) conditions. FIG. 9A depicts relatively low PAL and PheP production under aerobic conditions due to oxygen (O₂) preventing FNR from dimerizing and activating PAL and/or pheP gene expression. FIG. 9B depicts up-regulated PAL and PheP production under anacrobic conditions due to FNR dimerizing and inducing FNR promoter-mediated expression of PAL and pheP (squiggle above “PAL” and “pheP”). Arrows adjacent to a single rectangle, or a cluster of rectangles, depict the promoter responsible for driving transcription (in the direction of the arrow) of such gene(s). Arrows above each rectangle depict the expression product of each gene.

FIG. 10 depicts phenylalanine concentrations in cultures of synthetic probiotic strains, with and without an additional copy of pheP inserted on the chromosome. After 1.5 hrs of growth, cultures were placed in Coy anaerobic chamber supplying 90% N₂, 5% CO₂, and 5% H₂. After 4 hrs of induction, bacteria were resuspended in assay buffer containing 4 mM phenylalanine. Aliquots were removed from cell assays every 30 min for 3 hrs for phenylalanine quantification by mass spectrometry. Phenylalanine degradation rates in strains comprising an additional copy of pheP (SYN-PKU304 and SYN-PKU305; left) were higher than strains lacking an additional copy of pheP (SYN-PKU308 and SYN-PKU307; right).

FIG. 11. shows that pheP Overexpression Improves Phe Degradation. Strains containing different PAL genes and an additional copy of the gene encoding the pheP transporter were compared with strains lacking the additional pheP gene. Notably, the additional pheP copy permitted the complete degradation of 4 mM Phe in 4 hours in this experiment.

FIG. 12. Shows that cinnamate production is enhanced in pheP+Strains. Cinnamate production is directly correlated with Phe degradation. pheP+ refers to the Nissle parent strain containing an additional integrated copy of the pheP gene but lacking a PAL circuit.

FIG. 13 depicts diseases associated with branched chain amino acid degradative pathways.

FIG. 14 depicts aspects of the branched chain amino acid degradative pathway

FIG. 15 depicts aspects of the branched chain amino acid degradative pathway

FIG. 16 depicts aspects of the branched chain amino acid degradative pathway

FIG. 17 depicts possible components of a branched chain amino acid synthetic biotic disclosed herein

FIG. 18 depicts possible components of a branched chain amino acid synthetic biotic disclosed herein.

FIG. 19 depicts possible components of a branched chain amino acid synthetic biotic disclosed herein

FIG. 20 depicts one exemplary branched chain amino acid circuit. Genes shown are high affinity leucine transporter complex (LivKHMGF), the branched chain a-ketoacid decarboxylase (KivD) from Lactococcus lactis, aldehyde dehydrogenase 2 (Adh2) from Saccharomyces cerevisiae, and leucine dehydrogenase (Ldh) from Pseudomonas aeruginosa. The genes for the leucine exporter (LeuE) and ilvC have been deleted. The gene for ilvJ is added which can be under the control of the native, FNR, or constitutive promoter Ptac

FIG. 21 depicts exemplary components of a branched chain amino acid synthetic biotic disclosed herein for leucine degradation.

FIG. 22 depicts exemplary components of a branched chain amino acid synthetic biotic disclosed herein for leucine import.

FIG. 23 depicts one exemplary branched chain amino acid circuit. Genes shown are low affinity BCAA transporter (BrnQ), the branched chain a-ketoacid decarboxylase (KivD) from Lactococcus lactis, aldehyde dehydrogenase from E. Coli K-12 (PadA), and leuDH derived from Pseudomonas aeruginosa PA01 or Bacillus cereus. The genes for the leucine exporter (LeuE) and ilvC have been deleted. The gene for ilvJ is added.

FIG. 24 depicts one exemplary branched chain amino acid circuit. Genes shown are high affinity leucine transporter complex (LivKHMGF), the branched chain a-ketoacid decarboxylase (KivD) from Lactococcus lactis, aldehyde dehydrogenase from E. Coli K-12 (PadA), and leuDH derived from Pseudomonas aeruginosa PA01 or Bacillus cereus. The genes for the leucine exporter (LeuE) and ilvC have been deleted. The gene for BrnQ is added.

FIG. 25 depicts one exemplary branched chain amino acid circuit. Genes shown are high affinity leucine transporter complex (LivKHMGF), the branched chain a-ketoacid decarboxylase (KivD) from Lactococcus lactis, either aldehyde dehydrogenase from E. Coli K-12 (PadA), alcohol dehydrogenase YqhD from E. coli, or alcohol dehydrogenase Adh2 from S. cerevisiae, and L-AAD derived from Proteus vulgaris or Proteus mirabilis. The genes for the leucine exporter (LeuE) and ilvC have been deleted. The gene for BrnQ is added.

FIG. 26 depicts one exemplary branched chain amino acid circuit. Genes shown are high affinity leucine transporter complex (LivKHMGF), the branched chain a-ketoacid decarboxylase (KivD) from Lactococcus lactis, either aldehyde dehydrogenase from E. Coli K-12 (PadA), alcohol dehydrogenase YqhD from E. coli, or alcohol dehydrogenase Adh2 from S. cerevisiae, and L-AAD derived from Proteus vulgaris or Proteus mirabilis. The genes for the leucine exporter (LeuE) and ilvC have been deleted. The gene for BrnQ is added. The genes are under the control of the FNR promoter.

FIG. 27 depicts one exemplary branched chain amino acid circuit. Genes shown are high affinity leucine transporter complex (LivKHMGF), the branched chain a-ketoacid decarboxylase (KivD) from Lactococcus lactis, either aldehyde dehydrogenase from E. Coli K-12 (PadA), alcohol dehydrogenase YqhD from E. coli, or alcohol dehydrogenase Adh2 from S. cerevisiae, and LeuDh derived from Pseudomonas aeruginosa PA01 or Bacillus cereus. The genes for the leucine exporter (LeuE) and ilvC have been deleted. The gene for BrnQ is added. The genes are under the control of the FNR promoter.

FIG. 28 depicts one exemplary branched chain amino acid circuit. Genes shown are high affinity leucine transporter complex (LivKHMGF), the branched chain a-ketoacid decarboxylase (KivD) from Lactococcus lactis, either aldehyde dehydrogenase from E. Coli K-12 (PadA), alcohol dehydrogenase YqhD from E. coli, or alcohol dehydrogenase Adh2 from S. cerevisiae, and BCAA aminotransferase ilvE. The genes for the leucine exporter (LeuE) and ilvC have been deleted. The gene for BrnQ is added. The genes are under the control of the FNR promoter.

FIG. 29 depicts examples of circuit components for ldh, kivD and livKHMGF inducible expression in E. coli.

FIG. 30 depicts leucine levels in the Nissle ΔleuE deletion strain harboring a high-copy plasmid expressing kivD from the Tet promoter or further with a copy of the livKHMGF operon driven by the Tet promoter integrated into the chromosome at the lacZ locus, which were induced with ATC and incubated in culture medium supplemented with 2 mM leucine. Samples were removed at 0, 1.5, 6 and 18 h, and leucine concentration was determined by liquid chromatography tandem mass spectrometry.

FIG. 31 depicts leucine degradation in the Nissle ΔleuE deletion strain harboring a high-copy plasmid expressing the branch-chain keto-acid dehydrogenase (bkd) complex with or without expression of a leucine dehydrogenase (ldh) from the Tet promoter or further with a copy of the leucine importer livKHMGF driven by the Tet promoter integrated into the chromosome at the lacZ locus, which were induced with ATC and incubated in culture medium supplemented with 2 mM leucine. Samples were removed at 0, 1.5, 6 and 18 h, and leucine concentration was determined by liquid chromatography tandem mass spectrometry.

FIGS. 32A, 32B, and 32C depict the simultaneous degradation of leucine (FIG. 32A), isoleucine (FIG. 32B), and valine (FIG. 32C) by E. coli Nissle and its AlcuE deletion strain harboring a high-copy plasmid expressing the keto-acid decarboxylase kivD from the Tet promoter or further with a copy of the livKHMGF operon driven by the Tet promoter integrated into the chromosome at the lacZ locus, which were induced with ATC and incubated in culture medium supplemented with 2 mM leucine, 2 mM isoleucine and 2 mM valinc. Samples were removed at 0, 1.5, 6 and 18 h, and leucine concentration was determined by liquid chromatography tandem mass spectrometry.

FIG. 33 shows that overexpression of the low-affinity BCAA transporter BrnQ greatly improves the rate of leucine degradation in a LeuE and ilvC knockout bacterial strain having either LeuDH derived from P. aeruginosa or LeuDH derived from Bacillus cereus, kivD, and padA with and without the BCAA transporter brnQ under the control of tet promoter as measured by leucine degradation, KIC production, and isovalerate production.

FIG. 34 is schematic depicting an exemplary Adenosine Degradation Circuit. Adenosine is imported into the cell through expression of the E. coli Nucleoside Permease nupG transporter. Adenosine is converted to Inosine through expression of Adenine Deaminase add. Inosine is converted to hypoxyxanthine through expression of Inosine Phosphorylase, xapA, and deoD. Hypoxanthine is converted to Xanthine and Urate through expression of Hypoxanthine Hydroxylase, xdhA, xdhB, xdhC. All of these genes are optionally expressed from an inducible promoter, e.g., a FNR-inducible promoter. The bacteria may also include an auxotrophy, e.g., deletion of thyA (A thyA; thymindine dependence). Non-limiting example of a bacterial strain is listed.

FIG. 35. is a schematic depicting an exemplary circuit for depleting kynureninc.

FIG. 36. shows the results of an adaptive laboratory evolution to select a bacterial mutant with enhanced kynurenine import into the cell. The results of the initial checkerboard assay are displayed as a function of optical density at 600 nm. The X-axis shows decreasing KYNU concentration from left-to-right, while the Z-axis shows decreasing Tox Trp concentration from front-to-back with the very back row representing media with no ToxTrp.

FIG. 37. shows the results of an adaptive laboratory evolution to select a bacterial mutant with enhanced kynurenine import into the cell.

FIG. 38. shows the results of an adaptive laboratory evolution to select a bacterial mutant with enhanced kynurenine import into the cell. The control strains SYN094 and trpE are shown in M9+KYNU without any ToxTrp, as there was no growth detected from either strain at any concentration of Tox Trp. The results of the assay show that expression of the pseudoKYNase provides protection against toxicity of Tox Trp and shows that growth is permitted between 250-62.5 ug/mL of KYNU and 6.3-1.55 ug/mL of ToxTrp.

FIG. 39. is a schematic depicting an exemplary circuit for treating hepatic encephalopathy.

FIG. 40. is a schematic depicting an exemplary circuit for depleting bile salts.

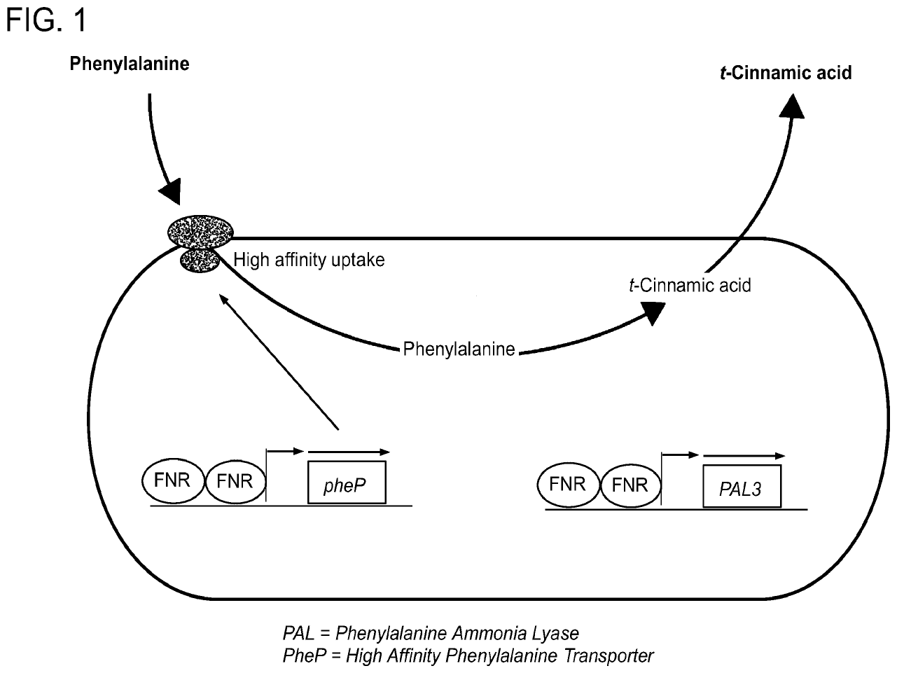

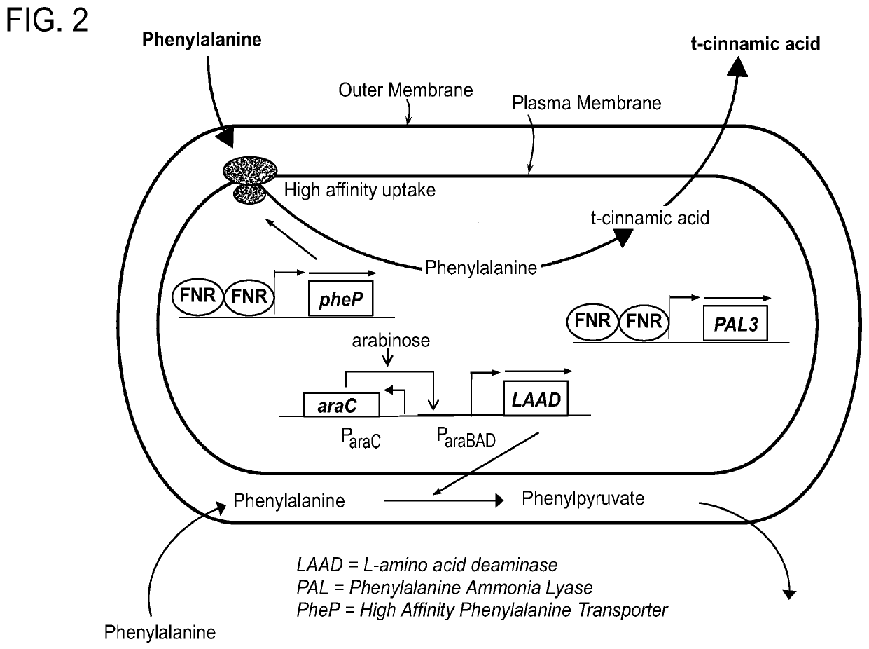

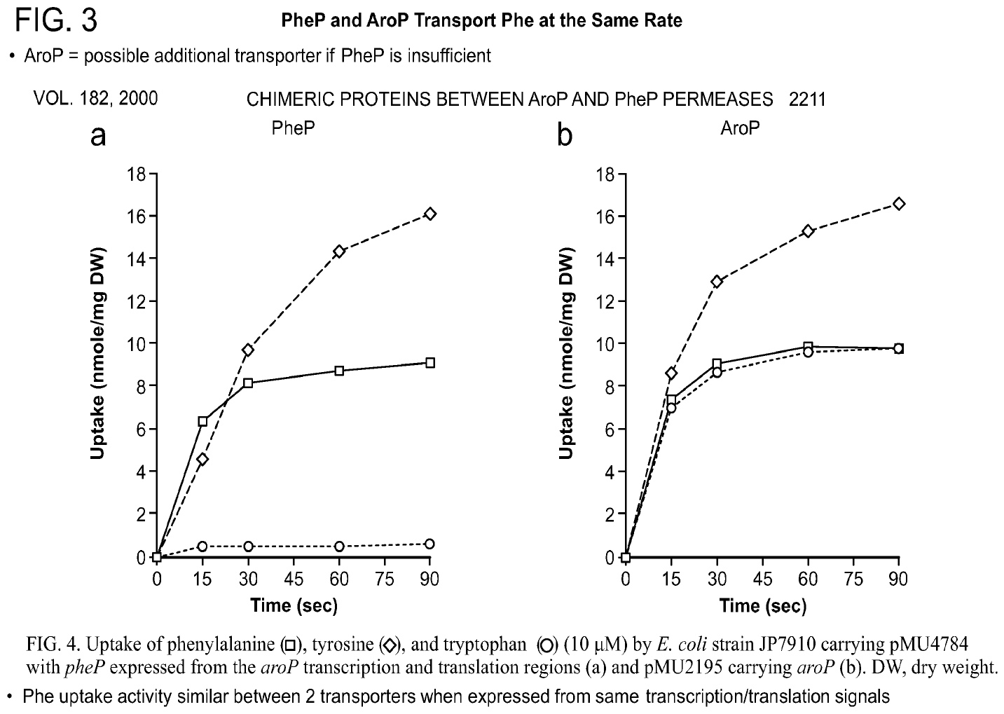

### DETAILED DESCRIPTION

The invention includes genetically engineered microorganisms, e.g., genetically engineered bacteria, pharmaceutical compositions thereof, and methods of modulating or treating a disease.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

As used herein, the term “amino acid” refers to a class of organic compounds that contain at least one amino group and one carboxyl group. Amino acids include leucine, isoleucine, valine, arginine, lysine, asparagine, serine, glycine, glutamine, tryptophan, methionine, threonine, cysteine, tyrosine, phenylalanine, glutamic acid, aspartic acid, alanine, histidine, and proline.

As used herein, the term “auxotroph” or “auxotrophic” refers to an organism that requires a specific factor, e.g., an amino acid, a sugar, or other nutrient, to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

“Cancer” or “cancerous” is used to refer to a physiological condition that is characterized by unregulated cell growth. In some embodiments, cancer refers to a tumor. “Tumor” is used to refer to any neoplastic cell growth or proliferation or any pre-cancerous or cancerous cell or tissue. A tumor may be malignant or benign. Types of cancer include, but are not limited to, adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hodgkin lymphoma, Non-Hodgkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor. Side effects of cancer treatment may include, but are not limited to, opportunistic autoimmune disorder(s), systemic toxicity, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration (National Cancer Institute).

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, and stem-loop structures. In one embodiment, the regulatory sequence comprises a promoter, e.g., an FNR responsive promoter.

As used herein the term “codon-optimized” refers to the modification of codons in a gene or a coding region of a nucleic acid molecule to improve translation in a host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Such optimization includes replacing at least one, or more than one, or a significant number, of codons with one or more codons that are more frequently used in the genes of the host organism.

Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (IRNA) molecules. The predominance of selected tRNAs in a cell is generally a reflection of the codons used most frequently in peptide synthesis. Accordingly, gencs can be tailored for optimal gene expression in a given organism based on codon optimization.

“Constitutive promoter” refers to a promoter that is capable of facilitating continuous transcription of a coding sequence or gene under its control and/or to which it is operably linked. Constitutive promoters and variants are well known in the art and include, but are not limited to, BBa_J23100, a constitutive Escherichia coli σ^{S} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_1712074; BBa_1719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

“Exogenous environmental condition(s)” refer to setting(s) or circumstance(s) under which the promoter described herein is induced. The phrase “exogenous environmental conditions” is meant to refer to the environmental conditions external to the intact (unlysed) recombinant micororganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are low-oxygen, microaerobic, or anaerobic conditions, such as hypoxic and/or necrotic tissues. In some embodiments, the exogenous environmental condition is a low-pH environment. In some embodiments, the recombinant bacterial cell of the disclosure comprise a pH-dependent promoter. In some embodiments, the recombinant bacterial cell of the disclosure comprise an oxygen level-dependent promoter. In some aspects, bacteria have evolved transcription factors that are capable of sensing oxygen levels (i.e., oxygen-level dependent transcription factors). Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase)-responsive promoters, ANR (anaerobic nitrate respiration)-responsive promoters, and DNR (dissimilatory nitrate respiration regulator)-responsive promoters. Multiple FNR-responsive promoters, ANR-responsive promoters, and DNR-responsive promoters which can be used in the present invention are known in the art (see, e.g., Castiglione et al. (2009) Microbiology 155 (Pt. 9): 2838-44; Eiglmeier et al. (1989) Mol. Microbiol. 3 (7): 869-78; Galimand et al. (1991) J. Bacteriol. 173 (5): 1598-1606; Hasegawa et al. (1998) FEMS Microbiol. Lett. 166 (2): 213-217; Hoeren et al. (1993) Eur. J. Biochem. 218 (1): 49-57; Salmon et al. (2003) J. Biol. Chem. 278 (32): 29837-55), and non-limiting examples are shown in Table 1.

TABLE 1
Examples of transcription factors and
responsive genes and regulatory regions

| Transcription | Examples of responsive genes, |
|---|---|
| Factor | promoters, and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

In a non-limiting example, a promoter (PfnrS) from the E. coli Nissle fumarate and nitrate reductase gene S (fnrS) that is known to be highly expressed under conditions of low or no environmental oxygen can be used in the present invention (Durand and Storz, 2010; Boysen et al, 2010). The PfnrS promoter is activated under anaerobic conditions by the global transcriptional regulator FNR that is naturally found in E. coli Nissle. Under anaerobic conditions, FNR forms a dimer and binds to specific sequences in the promoters of genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as “FNRS”, “fnrs”, “FNR”, “P-FNRS” promoter and other such related designations to indicate the promoter PfnrS.

As used herein, the term “expression” refers to the transcription and stable accumulation of sense (mRNA) or anti-sense RNA derived from a nucleic acid, and/or to translation of an mRNA into a polypeptide.

As used herein, the term “gene” refers to a nucleic acid fragment that encodes a protein or a fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequence and/or the regulatory sequence, in whole or in part, are not found together in nature. Thus, a chimeric gene may comprise a regulatory sequence and a coding sequence, each derived from different sources, or a regulatory and a coding sequence each derived from the same source, but arranged differently than they are found in nature.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising at least one substrate transporter operably linked to a promoter, into a bacterial cell. Genetic modification can also involve a targeted replacement in the chromosome, e.g., to replace a native gene promoter with an inducible promoter, regulated promoter, strong promoter, weak promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or multiple changes in a nucleotide sequence of a gene or related regulatory region that alters the nucleotide sequence as compared to its native or wild-type sequence. Mutations include, for example, substitutions, insertions, and deletions, in whole or in part, within the wild-type sequence. Such substitutions, insertions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., import activity) of the polypeptide product encoded by the gene. A genetic mutation in a regulatory sequence may increase, decrease, or otherwise alter the expression of sequences operably linked to the altered regulatory sequence.

It is routine for one of ordinary skill in the art to make mutations in a gene of interest. Mutations include substitutions, insertions, deletions, and/or truncations of one or more specific amino acid residues or of one or more specific nucleotides or codons in the polypeptide or polynucleotide of interest. Mutagenesis and directed evolution methods are well known in the art for creating variants. Sec, e.g., U.S. Pat. Nos. 7,783,428; 6,586,182; 6,117,679; and Ling, et al., 1999, “Approaches to DNA mutagenesis: an overview,” Anal. Biochem., 254 (2): 157-78; Smith, 1985, “In vitro mutagenesis,” Ann. Rev. Genet., 19:423-462; Carter, 1986, “Site-directed mutagenesis,” Biochem. J., 237:1-7; and Minshull, et al., 1999, “Protein evolution by molecular breeding,” Current Opinion in Chemical Biology, 3:284-290. For example, the lambda red system can be used to knock-out genes in E. coli (see, e.g., Datta et al., Gene, 379:109-115 (2006)).

“Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

As used herein, “heterologous” as used in the context of a nucleic acid or polypeptide sequence, “heterologous gene”, or “heterologous sequence”, refers to a nucleotide or polypeptide sequence that is not normally found in a given cell in nature. As used herein, a heterologous sequence encompasses a nucleic acid sequence that is exogenously introduced into a given cell. “Heterologous gene” includes a native gene, or fragment thereof, that has been introduced into the host cell in a form that is different from the corresponding native gene. For example, a heterologous gene may include a native coding sequence that is a portion of a chimeric gene to include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

The term “inactivated” as applied to a gene refers to any genetic modification that decreases or eliminates the expression of the gene and/or the functional activity of the corresponding gene product (mRNA and/or protein). The term “inactivated” encompasses complete or partial inactivation, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

An “inducible promoter” refers to a regulatory nucleic acid region that is operably linked to one or more genes, wherein transcription of the gene(s) is increased in response to a stimulus (e.g., an inducer) or an exogenous environmental condition. A “directly inducible promoter” refers to a regulatory region, wherein the regulatory region is operably linked to a gene encoding a protein or polypeptide, where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Examples of inducible promoters include, but are not limited to, an FNR promoter, a P_{araC} promoter, a P_{araBAD} promoter, a propionate promoter, and a P_{TetR} promoter, each of which are described in more detail herein. Examples of other inducible promoters are provided herein below.

An “isolated” polypeptide, or a fragment, variant, or derivative thereof, refers to a polypeptide that is not in its natural milieu. No particular level of purification is required. Recombinantly-produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment.” “variant.” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 2014/0079701, the contents of which are herein incorporated by reference in its entirety.

As used herein, the terms “modulate” and “treat” and their cognates refer to an amelioration of a disease or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease or condition, either physically (e.g., stabilization of a discernible symptom), physiologically (e.g., stabilization of a physical parameter), or both. In another embodiment, “modulate” and “treat” refer to slowing the progression or reversing the progression of a disease or condition. As used herein, “prevent” and its cognates refer to delaying the onset or reducing the risk of acquiring a given disease or condition.

As used herein, a “non-native” nucleic acid sequence refers to a nucleic acid sequence not normally present in a microorganism, e.g., an extra copy of an endogenous sequence, or a heterologous sequence such as a sequence from a different organism (e.g., an organism from a different species, strain, or substrain of a prokaryote or eukaryote), or a sequence that is modified and/or mutated as compared to the unmodified native or wild-type sequence. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes (e.g., genes in a gene cassette or operon). In some embodiments, “non-native” refers to two or more nucleic acid sequences that are not found in the same relationship to each other in nature. The non-native nucleic acid sequence may be present on a plasmid or chromosome. In some embodiments, the genetically engineered bacteria of the disclosure comprise a gene that is operably linked to a directly or indirectly inducible promoter that is not associated with said gene in nature, e.g., an FNR-responsive promoter (or other promoter described herein) operably linked to a gene encoding a substrate transporter.

“Microorganism” refers to an organism or microbe of microscopic, submicroscopic, or ultramicroscopic size that typically consists of a single cell. Examples of microrganisms include bacteria, viruses, parasites, fungi, certain algae, and protozoa. In some aspects, the microorganism is engineered (“engineered microorganism”) to produce one or more anti-cancer molecules. In certain embodiments, the engineered microorganism is an engineered bacteria. In certain embodiments, the engineered microorganism is an engineered oncolytic virus.

“Non-pathogenic bacteria” refer to bacteria that are not capable of causing disease or harmful responses in a host. In some embodiments, non-pathogenic bacteria are commensal bacteria. Examples of non-pathogenic bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, and Lactococcus lactis (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence that is joined to a regulatory sequence in a manner which allows expression of the nucleic acid sequence. In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes.

As used herein, “payload” refers to one or more molecules of interest to be produced by a genetically engineered microorganism, such as a bacteria. In some embodiments, the payload is a therapeutic payload. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments the payload comprises a repressor element, such as a kill switch. In some embodiments, the payload is encoded by a gene or multiple genes or an operon. In alternate embodiments, the payload is produced by a biosynthetic or biochemical pathway, wherein the biosynthetic or biochemical pathway may optionally be endogenous to the microorganism. In some embodiments, the genetically engineered microorganism comprises two or more payloads.

As used herein a “pharmaceutical composition” refers to a preparation of bacterial cells disclosed herein with other components such as a physiologically suitable carrier and/or excipient.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial compound. An adjuvant is included under these phrases.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene encoding at least one substrate transporter.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length. Thus, “peptides,” “dipeptides,” “tripeptides, “oligopeptides,” “protein.” “amino acid chain,” or any other term used to refer to a chain or chains of two or more amino acids, are included within the definition of “polypeptide,” and the term “polypeptide” may be used instead of, or interchangeably with, any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria of the current invention. In some embodiments, a polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they must not necessarily have such structure. Polypeptides with a defined three-dimensional structure are referred to as folded, and polypeptides, which do not possess a defined three-dimensional structure, but rather can adopt a large number of different conformations, are referred to herein as unfolded.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: −Ala, Pro, Gly, Gln, Asn, Scr, Thr; −Cys, Ser, Tyr, Thr; −Val, Ile. Leu, Met, Ala, Phe; −Lys, Arg, His; −Phe, Tyr, Trp, His; and −Asp, Glu.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Bifidobacteria, Escherichia coli, and Lactobacillus, e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive.

As used herein, the term “recombinant bacterial cell”, “recombinant bacteria” or “genetically modified bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells of the disclosure may comprise exogenous nucleotide sequences on plasmids. Alternatively, recombinant bacterial cells may comprise exogenous nucleotide sequences stably incorporated into their chromosome.

As used herein, “stably maintained” or “stable” bacterium is used to refer to a bacterial host cell carrying non-native genetic material, e.g., an amino acid catabolism enzyme, that is incorporated into the host genome or propagated on a self-replicating extrachromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising an substrate transporter gene, in which the plasmid or chromosome carrying the substrate transporter gene is stably maintained in the bacterium, such that the substrate transporter can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

The terms “therapeutically effective dose” and “therapeutically effective amount” are used to refer to an amount of a compound that results in prevention, delay of onset of symptoms, or amelioration of symptoms of a condition or disease. A therapeutically effective amount, as well as a therapeutically effective frequency of administration, can be determined by methods known in the art and discussed below.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “treat” and its cognates refer to an amelioration of a disease, or at least one discernible symptom thereof. In another embodiment, “treat” refers to an amelioration of at least one measurable physical parameter, not necessarily discernible by the patient. In another embodiment, “treat” refers to inhibiting the progression of a disease, either physically (e.g., stabilization of a discernible symptom), physiologically (e.g., stabilization of a physical parameter), or both. In another embodiment, “treat” refers to slowing the progression or reversing the progression of a disease. As used herein, “prevent” and its cognates refer to delaying the onset or reducing the risk of acquiring a given disease.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Disorders associated with or involved with amino acid metabolism, e.g., cancer, may be caused by inborn genetic mutations for which there are no known cures. Diseases can also be secondary to other conditions, e.g., an intestinal disorder or a bacterial infection. Treating diseases associated with amino acid metabolism may encompass reducing normal levels of one or more substrates, e.g., an amino acid, reducing excess levels of one or more substrates, e.g., an amino acid, or eliminating one or more substrates, e.g., an amino acid, and does not necessarily encompass the elimination of the underlying disease.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Bacterial Strains

The disclosure provides a bacterial cell that comprises a heterologous gene encoding a substrate transporter. In some embodiments, the bacterial cell is a non-pathogenic bacterial cell. In some embodiments, the bacterial cell is a commensal bacterial cell. In some embodiments, the bacterial cell is a probiotic bacterial cell.

In certain embodiments, the bacterial cell is selected from the group consisting of a Bacteroides fragilis, Bacteroides thetaiotaomicron, Bacteroides subtilis, Bifidobacterium animalis, Bifidobacterium bifidum, Bifidobacterium infantis, Bifidobacterium lactis, Clostridium butyricum, Clostridium scindens, Escherichia coli, Lactobacillus acidophilus, Lactobacillus plantarum, Lactobacillus reuteri, Lactococcus lactis, and Oxalobacter formigenes bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium animalis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis bacterial cell. In one embodiment, the bacterial cell is a Clostridium butyricum bacterial cell. In one embodiment, the bacterial cell is a Clostridium scindens bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a Oxalobacter formigenes bacterial cell. In another embodiment, the bacterial cell does not include Oxalobacter formigenes.

In one embodiment, the bacterial cell is a Gram positive bacterial cell. In another embodiment, the bacterial cell is a Gram negative bacterial cell.

In some embodiments, the bacterial cell is Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its “complete harmlessness” (Schultz, 2008), and “has GRAS (generally recognized as safe) status” (Reister et al., 2014, emphasis added). Genomic sequencing confirmed that E. coli Nissle “lacks prominent virulence factors (e.g., E. coli α-hemolysin, P-fimbrial adhesins)” (Schultz, 2008), and E. coli Nissle “does not carry pathogenic adhesion factors and does not produce any enterotoxins or cytotoxins, it is not invasive, not uropathogenic” (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's “therapeutic efficacy and safety have convincingly been proven” (Ukena et al., 2007).

In one embodiment, the recombinant bacterial cell of the disclosure does not colonize the subject to whom the cell is administered.

One of ordinary skill in the art would appreciate that the genetic modifications disclosed herein may be adapted for other species, strains, and subtypes of bacteria. Furthermore, genes from one or more different species can be introduced into one another.

In some embodiments, the bacterial cell is a genetically engineered bacterial cell. In another embodiment, the bacterial cell is a recombinant bacterial cell. In some embodiments, the disclosure comprises a colony of recombinant bacterial cells.

In another aspect, the disclosure provides a recombinant bacterial culture which comprises bacterial cells disclosed herein. In one aspect, the disclosure provides a recombinant bacterial culture which reduces levels of a substrate, e.g., an amino acid or a peptide, in the media of the culture. In one embodiment, the levels of substrate is reduced by about 50%, by about 60%, by about 70%, by about 75%, by about 80%, by about 90%, by about 95%, or about 100% in the media of the cell culture. In another embodiment, the levels of a substrate is reduced by about two-fold, three-fold, four-fold, five-fold, six-fold, seven-fold, eight-fold, nine-fold, ten-fold, fifteen-fold, twenty-fold, thirty-fold, forty-fold, or fifty-fold, in the media of the cell culture. In one embodiment, the levels of a substrate are reduced below the limit of detection in the media of the cell culture.

In some embodiments of the above described recombinant bacterial cells, the gene encoding a substrate transporter is present on a plasmid in the bacterium and operatively linked on the plasmid to the promoter that is induced under low-oxygen or anaerobic conditions. In other embodiments, the gene encoding a substrate transporter is present in the bacterial chromosome and is operatively linked in the chromosome to the promoter that is induced under low-oxygen or anaerobic conditions.

In some embodiments, the recombinant bacterial cell comprising a heterologous substrate transporter is an auxotroph. In one embodiment, the recombinant bacterial cell is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the recombinant bacterial cell has more than one auxotrophy, for example, they may be a ΔthyA and ΔdapA auxotroph.

In some embodiments, the recombinant bacterial cell comprising a heterologous substrate transporter further comprises a kill-switch circuit, such as any of the kill-switch circuits provided herein. For example, in some embodiments, the recombinant bacterial cells may further comprise one or more genes encoding one or more recombinase(s) under the control of an inducible promoter, and an inverted toxin sequence. In some embodiments, the recombinant bacterial cell further comprises one or more genes encoding an antitoxin. In some embodiments, the recombinant bacterial cell further comprise one or more genes encoding one or more recombinase(s) under the control of an inducible promoter and one or more inverted excision genes, wherein the excision gene(s) encode an enzyme that deletes an essential gene. In some embodiments, the recombinant bacterial cell further comprise one or more genes encoding an antitoxin. In some embodiments, the recombinant bacterial cell further comprises one or more genes encoding a toxin under the control of an promoter having a TetR repressor binding site and a gene encoding the TetR under the control of an inducible promoter that is induced by arabinose, such as P_{araBAD}. In some embodiments, the recombinant bacterial cell further comprises one or more genes encoding an antitoxin.

In some embodiments, the recombinant bacterial cell is an auxotroph comprising a heterologous substrate transporter gene and further comprises a kill-switch circuit, such as any of the kill-switch circuits described herein.

In some embodiments of the above described recombinant bacterial cell, the heterologous gene encoding a substrate transporter is present on a plasmid in the bacterium and operatively linked on the plasmid to the promoter that is induced under low-oxygen or anaerobic conditions. In other embodiments, the gene encoding a substrate transporter is present in the bacterial chromosome and is operatively linked in the chromosome to the promoter that is induced under low-oxygen or anaerobic conditions.

### A. Amino Acid Transporters

In one embodiment, the recombinant bacterial cell of the invention comprises a heterologous gene encoding a substrate transporter, wherein the substrate transporter is an amino acid transporter. In one embodiment, the amino acid transporter transports at least one amino acid selected from the group consisting of leucine, isoleucine, valine, arginine, lysine, asparagine, serine, glycine, glutamine, tryptophan, methionine, threonine, cysteine, tyrosine, phenylalanine, glutamic acid, aspartic acid, alanine, histidine, and proline, into the cell.

The uptake of amino acids into bacterial cells is mediated by proteins well known to those of skill in the art. Amino acid transporters may be expressed or modified in the bacteria in order to enhance amino acid transport into the cell. Specifically, when the amino acid transporter is expressed in the recombinant bacterial cells, the bacterial cells import more amino acid(s) into the cell when the transporter is expressed than unmodified bacteria of the same bacterial subtype under the same conditions. In one embodiment, the bacterial cell comprises a heterologous gene encoding an amino acid transporter. In one embodiment, the bacterial cell comprises a heterologous gene encoding an amino acid transporter and a genetic modification that reduces export of an amino acid, e.g., a genetic mutation in an exporter gene or promoter.

In one embodiment, the bacterial cell comprises at least one gene encoding an amino acid transporter from a different organism, e.g., a different species of bacteria. In one embodiment, the bacterial cell comprises at least one native gene encoding an amino acid transporter. In some embodiments, the at least one native gene encoding an amino acid transporter is not modified. In another embodiment, the bacterial cell comprises more than one copy of at least one native gene encoding an amino acid transporter. In yet another embodiment, the bacterial cell comprises a copy of at least one gene encoding a native amino acid transporter, as well as at least one copy of at least one heterologous gene encoding anamino acid transporter from a different bacterial species. In one embodiment, the bacterial cell comprises at least one, two, three, four, five, or six copies of the at least one heterologous gene encoding an amino acid transporter. In one embodiment, the bacterial cell comprises multiple copies of the at least one heterologous gene encoding an amino acid transporter.

In one embodiment, the recombinant bacterial cell comprises a heterologous gene encoding an amino acid transporter, wherein said amino acid transporter comprises an amino acid sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the amino acid sequence of a polypeptide encoded by an amino acid transporter gene disclosed herein.

In some embodiments, the amino acid transporter is encoded by an amino acid transporter gene derived from a bacterial genus or species, including but not limited to, Bacillus, Campylobacter, Clostridium, Escherichia, Lactobacillus, Pseudomonas, Salmonella, Staphylococcus, Bacillus subtilis, Campylobacter jejuni, Clostridium perfringens, Escherichia coli, Lactobacillus delbrueckii, Pseudomonas aeruginosa, Salmonella typhimurium, or Staphylococcus aureus. In some embodiments, the bacterial species is Escherichia coli. In some embodiments, the bacterial species is Escherichia coli strain Nissle.

The present disclosure further comprises genes encoding functional fragments of an amino acid transporter or functional variants of an amino acid transporter. As used herein, the term “functional fragment thereof” or “functional variant thereof” of an amino acid transporter relates to an element having qualitative biological activity in common with the wild-type amino acid transporter from which the fragment or variant was derived. For example, a functional fragment or a functional variant of a mutated amino acid transporter is one which retains essentially the same ability to import an amino acid into the bacterial cell as does the amino acid transporter protein from which the functional fragment or functional variant was derived. In one embodiment, the recombinant bacterial cell comprises at least one heterologous gene encoding a functional fragment of an amino acid transporter. In another embodiment, the recombinant bacterial cell comprises a heterologous gene encoding a functional variant of an amino acid transporter.

Assays for testing the activity of an amino acid transporter, a functional variant of an amino acid transporter, or a functional fragment of an amino acid transporter are well known to one of ordinary skill in the art. For example, import of an amino acid may be determined using the methods as described in Haney et al., J. Bact., 174 (1): 108-15, 1992; Rahmanian et al., J. Bact., 116 (3): 1258-66, 1973; and Ribardo and Hendrixson, J. Bact., 173 (22): 6233-43, 2011, the entire contents of each of which are expressly incorporated by reference herein.

In one embodiment, the genes encoding the amino acid transporter have been codon-optimized for use in the host organism, e.g., a bacterial cell disclosed herein. In one embodiment, the genes encoding the amino acid transporter have been codon-optimized for use in Escherichia coli.

The present disclosure also encompasses genes encoding an amino acid transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding an amino acid transporter is mutagenized; mutants exhibiting increased amino acid import are selected; and the mutagenized at least one gene encoding an amino acid transporter is isolated and inserted into the bacterial cell. In some embodiments, the at least one gene encoding an amino acid transporter is mutagenized; mutants exhibiting decreased amino acid import are selected; and the mutagenized at least one gene encoding an amino acid transporter is isolated and inserted into the bacterial cell. The transporter modifications described herein may be present on a plasmid or chromosome.

In some embodiments, the bacterial cell comprises a heterologous gene encoding an amino acid transporter operably linked to a promoter. In one embodiment, the at least one gene encoding an amino acid transporter is directly operably linked to the promoter. In another embodiment, the at least one gene encoding an amino acid transporter is indirectly operably linked to the promoter.

In one embodiment, the promoter is not operably linked with the at least one gene encoding an amino acid transporter in nature. In some embodiments, the at least one gene encoding the amino acid transporter is controlled by its native promoter. In some embodiments, the at least one gene encoding the amino acid transporter is controlled by an inducible promoter. In some embodiments, the at least one gene encoding the amino acid transporter is controlled by a promoter that is stronger than its native promoter. In some embodiments, the at least one gene encoding the amino acid transporter is controlled by a constitutive promoter.

In another embodiment, the promoter is an inducible promoter. Inducible promoters are described in more detail infra.

In one embodiment, the at least one gene encoding an amino acid transporter is located on a plasmid in the bacterial cell. In some embodiments, the plasmid is a high copy number plasmid. In some embodiments, the plasmid is a low copy number plasmid. In another embodiment, the at least one gene encoding an amino acid transporter is located in the chromosome of the bacterial cell. In yet another embodiment, a native copy of the at least one gene encoding an amino acid transporter is located in the chromosome of the bacterial cell, and a copy of at least one gene encoding an amino acid transporter from a different species of bacteria is located on a plasmid in the bacterial cell. In yet another embodiment, a native copy of the at least one gene encoding an amino acid transporter is located on a plasmid in the bacterial cell, and a copy of at least one gene encoding an amino acid transporter from a different species of bacteria is located on a plasmid in the bacterial cell. In yet another embodiment, a native copy of the at least one gene encoding an amino acid transporter is located in the chromosome of the bacterial cell, and a copy of the at least one gene encoding an amino acid transporter from a different species of bacteria is located in the chromosome of the bacterial cell.

In some embodiments, the at least one native gene encoding the amino acid transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the amino acid transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the amino acid transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the amino acid transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the amino acid transporter is expressed in the recombinant bacterial cells, the bacterial cells import 10% more amino acids into the bacterial cell when the transporter is expressed than unmodified bacteria of the same bacterial subtype under the same conditions. In another embodiment, when the amino acid transporter is expressed in the recombinant bacterial cells, the bacterial cells import 20%, 30%, 40%, 50%, 60%, 70%, 80%, 90% or 100% more amino acids, into the bacterial cell when the transporter is expressed than unmodified bacteria of the same bacterial subtype under the same conditions. In yet another embodiment, when the amino acid transporter is expressed in the recombinant bacterial cells, the bacterial cells import two-fold more amino acids into the cell when the transporter is expressed than unmodified bacteria of the same bacterial subtype under the same conditions. In yet another embodiment, when the amino acid transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more amino acid into the cell when the transporter is expressed than unmodified bacteria of the same bacterial subtype under the same conditions.

In one embodiment, the recombinant bacterial cells described herein comprise a first heterologous amino acid transporter and a second heterologous amino acid transporter. In one embodiment, said first amino acid transporter is derived from a different organism than said second amino acid transporter. In some embodiments, said first amino acid transporter is derived from the same organism as said second amino acid transporter. In some embodiments, said first amino acid transporter imports the same amino acid as said second amino acid transporter. In other embodiment, said first amino acid transporter imports a different amino acid from said second amino acid transporter. In some embodiments, said first amino acid transporter is a wild-type amino acid transporter and said second amino acid transporter is a mutagenized version of said first amino acid transporter. In some embodiments, the recombinant bacterial cells described herein comprise at least a third heterologous amino acid transporter. In some embodiments, the recombinant bacterial cells described herein comprise at least four heterologous amino acid transporters. In some embodiments, the recombinant bacterial cells described herein comprise at least five heterologous amino acid transporters or more.

In one embodiment, the amino acid transporter imports one amino acid into the bacterial cell. In another embodiment, the amino acid transporter imports two amino acids into the bacterial cell. In yet another embodiment, the amino acid transporter imports three amino acids into the bacterial cell. In another embodiment, the amino acid transporter imports four or more amino acids into the cell. In one embodiment, the amino acid transporter is an arginine transporter. In another embodiment, the amino acid transporter is an asparagine transporter. In another embodiment, the amino acid transporter is a serine transporter. In another embodiment, the amino acid transporter is an transporter of glycine. In another embodiment, the amino acid transporter is a tryptophan transporter. In another embodiment, the amino acid transporter is a methionine transporter. In another embodiment, the amino acid transporter is a threonine transporter. In another embodiment, the amino acid transporter is a cysteine transporter. In another embodiment, the amino acid transporter is a tyrosine transporter. In another embodiment, the amino acid transporter is a phenylalanine transporter. In another embodiment, the amino acid transporter is a glutamic acid transporter. In another embodiment, the amino acid transporter is a histidine transporter. In another embodiment, the amino acid transporter is a proline transporter. In another embodiment, the amino acid transporter is an transporter of leucine. In another embodiment, the amino acid transporter is an transporter of isoleucine. In another embodiment, the amino acid transporter is an transporter of valine. In another embodiment, the amino acid transporter is a lysine transporter. In another embodiment, the amino acid transporter is a glutamine transporter. In another embodiment, the amino acid transporter is an transporter of aspartic acid. In another embodiment, the amino acid transporter is an transporter of alanine. In another embodiment, the amino acid transporter is an transporter of branched chain amino acids.

In some embodiment, the recombinant bacterial cell comprising a heterologous gene encoding an amino acid transporter may be used to treat a disease, condition, and/or symptom associated with amino acid metabolism. In some embodiments, disclosed herein are methods for reducing, ameliorating, or eliminating one or more symptom(s) associated with these diseases or disorders.

As used herein the terms “disease associated with amino acid metabolism” or a “disorder associated with amino acid metabolism” is a disease or disorder involving the abnormal, e.g., increased, levels of one or more amino acids in a subject. In one embodiment, a disease or disorder associated with amino acid metabolism is a cancer, e.g., a cancer described herein. In another embodiment, a disease or disorder associated with amino acid metabolism is a metabolic disease. In one embodiment, the cancer is glioma. In another embodiment, the cancer is breast cancer. In another embodiment, the cancer is melanoma. In another embodiment, the cancer is hepatocarcinoma. In another embodiment, the cancer is acute lymphoblastic leukemia (ALL). In another embodiment, the cancer is ovarian cancer. In another embodiment, the cancer is prostate cancer. In another embodiment, the cancer is lymphoblastic leukemia. In another embodiment, the cancer is non-small cell lung cancer.

Multiple distinct transporters of amino acids are well known in the art and are described in the subsections, below.

### 1. Branched Chain Amino Acid Transporters

In one embodiment, the amino acid transporter is a branched chain amino acid transporter. The term “branched chain amino acid” or “BCAA,” as used herein, refers to an amino acid which comprises a branched side chain. Leucine, isoleucine, and valine are naturally occurring amino acids comprising a branched side chain. However, non-naturally occurring, usual, and/or modified amino acids comprising a branched side chain are also encompassed by the term branched chain amino acid.

Branched chain amino acid transporters may be expressed or modified in the recombinant bacteria described herein in order to enhance branched chain amino acid transport into the cell. Specifically, when the transporter of branched chain amino acids is expressed in the recombinant bacterial cells described herein, the bacterial cells import more branched chain amino acids into the cell when the transporter is expressed than unmodified bacteria of the same bacterial subtype under the same conditions. Thus, the genetically engineered bacteria comprising a heterologous gene encoding an transporter of branched chain amino acids may be used to import one or more branched chain amino acids into the bacteria.

The uptake of branched chain amino acids into bacterial cells is mediated by proteins well known to those of skill in the art. For example, two well characterized BCAA transport systems have been characterized in several bacteria, including Escherichia coli. BCAAs are transported by two systems into bacterial cells (i.e., imported), the osmotic-shock-sensitive systems designated LIV-I and LS (leucine-specific), and by an osmotic-shock resistant system, BrnQ, formerly known as LIV-II (see Adams et al., J. Biol. Chem. 265:11436-43 (1990); Anderson and Oxender, J. Bacteriol. 130:384-92 (1977); Anderson and Oxender, J. Bacteriol. 136:168-74 (1978); Hancy et al., J. Bacteriol. 174:108-15 (1992); Landick and Oxender, J. Biol. Chem. 260:8257-61 (1985); Nazos et al., J. Bacteriol. 166:565-73 (1986); Nazos et al., J. Bacteriol. 163:1196-202 (1985); Oxender et al., Proc. Natl. Acad. Sci. USA 77:1412-16 (1980); Quay et al., J. Bacteriol. 129:1257-65 (1977); Rahmanian et al., J. Bacteriol. 116:1258-66 (1973); Wood, J. Biol. Chem. 250:4477-85 (1975); Guardiola et al., J. Bacteriol. 117:393-405 (1974); Guardiola and Iaccarino, J. Bacteriol. 108:1034-44 (1971); Ohnishi et al., Jpn. J. Genet. 63:343-57) (1988); Yamato and Anraku, J. Bacteriol. 144:36-44 (1980); and Yamato et al., J. Bacteriol. 138:24-32 (1979)). Transport by the BrnQ system is mediated by a single membrane protein. Transport mediated by the LIV-I system is dependent on the substrate binding protein LivJ (also known as LIV-BP), while transport mediated by LS system is mediated by the substrate binding protein LivK (also known as LS-BP). LivJ is encoded by the liv gene, and binds isoleucine, leucine and valine with K_{d} values of ˜10⁻⁶ and ˜10⁻⁷ M, while LivK is encoded by the livK gene, and binds leucine with a K_{d} value of ˜10⁻⁶ M (See Landick and Oxender, J. Biol. Chem. 260:8257-61 (1985)). Both LivJ and LivK interact with the inner membrane components LivHMGF to enable ATP-hydrolysis-coupled transport of their substrates into the cell, forming the LIV-I and LS transport systems, respectively. The LIV-I system transports leucine, isoleucine and valine, and to a lesser extent serine, threonine and alanine, whereas the LS system only transports leucine. The six genes encoding the E. coli LIV-I and LS systems are organized into two transcriptional units, with livKHMGF transcribed as a single operon, and liv transcribed separately. The Escherichia coli liv genes can be grouped according to protein function, with the livJ and livK genes encoding periplasmic binding proteins with the binding affinities described above, the livH and livM genes encoding inner membrane permeases, and the livG and livF genes encoding cytoplasmic ATPases.

In one embodiment, the at least one gene encoding an branched chain amino acid transporter is the brnQ gene. An exemplary sequence for brnQ is provided below. BCAA transporter BrnQ from E. coli:

Nucleotide sequence:
atgacccatcaattaagatcgcgcgatatcatcgctctgggctttatgacatttgcgttgttcgtcggcgcaggtaacattattttccctcc
aatggtcggcttgcaggcaggcgaacacgtctggactgcggcattcggcttcctcattactgccgttggcctaccggtattaacggtagtggc
gctggcaaaagttggcggcggtgttgacagtctcagcacgccaattggtaaagtcgctggcgtactgctggcaacagtttgttacctggc
ggtggggccgctttttgctacgccgcgtacagctaccgtttcttttgaagtgggcattgcgccgctgacgggtgattccgcgctgccgctgt
ttatttacagcctggtctatttcgctatcgttattctggtttcgctctatccgggcaagctgctggataccgtgggcaacttccttgcgccgc
tgaaaattatcgcgctggtcatcctgtctgttgccgcaattatctggccgggggttctatcagtacggcgactgaggcttatcaaaacgctgc
gttttctaacggcttcgtcaacggctatctgaccatggatacgctgggcgcaatggtgtttggtatcgttattgttaacgcggcgcgttctcg
tggcgttaccgaagcgcgtctgctgacccgttataccgtctgggctggcctgatggcgggtgttggtctgactctgctgtacctggcgctgtt
ccgtctgggttcagacagcgcgtcgctggtcgatcagtctgcaaacggtgcggcgatcctgcatgcttacgttcagcatacctttggcggc
ggcggtagcttcctgctggcggcgttaatcttcatcgcctgcctggtcacggcggttggcctgacctgtgcttgtgcagaattcttcgccca
gtacgtaccgctctcttatcgtacgctggtgtttatcctcggcggcttctcgatggtggtgtctaacctcggcttgagccagctgattcagat
ctctgtaccggtgctgaccgccatttatccgccgtgtatcgcactggttgtattaagttttacacgctcatggtggcataattcgtcccgcgt
gattgctccgccgatgtttatcagcctgctttttggtattctcgacgggatcaaggcatctgcattcagcgatatcttaccgtcctgggcgca
gcgtttaccgctggccgaacaaggtctggcgtggttaatgccaacagtggtgatggtggttctggccattatctgggatcgtgcggcaggtcg
tcaggtgacctccagcgctcactaa
AA sequence:
MTHQLRSRDIIALGFMTFALFVGAGNIIFPPMVGLQAGEHVWTAAFGFLITAVGLPVLT
VVALAKVGGGVDSLSTPIGKVAGVLLATVCYLAVGPLFATPRTATVSFEVGIAPLTGD
SALPLFIYSLVYFAIVILVSLYPGKLLDTVGNFLAPLKIIALVILSVAAIIWPAGSISTATE
AYQNAAFSNGFVNGYLTMDTLGAMVFGIVIVNAARSRGVTEARLLTRYTVWAGLMA
GVGLTLLYLALFRLGSDSASLVDQSANGAAILHAYVQHTFGGGGSFLLAALIFIACLVT
AVGLTCACAEFFAQYVPLSYRTLVFILGGFSMVVSNLGLSQLIQISVPVLTAIYPPCIAL
VVLSFTRSWWHNSSRVIAPPMFISLLFGILDGIKASAFSDILPSWAQRLPLAEQGLAWL
MPTVVMVVLAIIWDRAAGRQVTSSAH

In one embodiment, the at least one gene encoding an branched chain amino acid transporter is the livJ gene. In one embodiment, the at least one gene encoding an branched chain amino acid transporter is the livH gene. In one embodiment, the at least one gene encoding an branched chain amino acid transporter is the livM gene. In one embodiment, the at least one gene encoding an branched chain amino acid transporter is the livG gene. In one embodiment, the at least one gene encoding an branched chain amino acid transporter is the livF gene. In one embodiment, the at least one gene encoding an branched chain amino acid transporter is the livKHMGF operon. In one embodiment, the at least one gene encoding an branched chain amino acid transporter is the livK gene. In another embodiment, the livKHMGF operon is an Escherichia coli livKHMGF operon. In another embodiment, the at least one gene encoding an branched chain amino acid transporter comprises the livKHMGF operon and the liv gene. In one embodiment, the bacterial cell of the invention has been genetically engineered to comprise at least one heterologous gene encoding a LIV-I system. In one embodiment, the bacterial cell of the invention has been genetically engineered to comprise at least one heterologous gene encoding a LS system. In one embodiment, the bacterial cell of the invention has been genetically engineered to comprise at least one heterologous gene encoding a LIV-I system. In one embodiment, the bacterial cell of the invention has been genetically engineered to comprise at least one heterologous livJ gene, and at least one heterologous gene selected from the group consisting of livH, livM, livG, and livF. In one embodiment, the bacterial cell of the invention has been genetically engineered to comprise at least one heterologous livK gene, and at least one heterologous gene selected from the group consisting of livH, livM, livG, and livF.

In one embodiment, the branched chain amino acid transporter gene has at least about 80% identity with the uppercase sequence of SEQ ID NO:9. Accordingly, in one embodiment, the branched chain amino acid transporter gene has at least about 90% identity with the uppercase sequence of SEQ ID NO:9. Accordingly, in one embodiment, the branched chain amino acid transporter gene has at least about 95% identity with the uppercase sequence of SEQ ID NO:9. Accordingly, in one embodiment, the branched chain amino acid transporter gene has at least about 70%, 75%, 80%, 85%, 86%, 87%, 88%, 89%, 90%, 91%, 92%, 93%, 94%, 95%, 96%, 97%, 98%, or 99% identity with the uppercase sequence of SEQ ID NO:9. In another embodiment, the branched chain amino acid transporter gene comprises the uppercase sequence of SEQ ID NO:9. In yet another embodiment the branched chain amino acid transporter gene consists of the uppercase sequence of SEQ ID NO:9.

In one embodiment, the branched chain amino acid transporter gene has at least about 80% identity with the sequence of SEQ ID NO:10. Accordingly, in one embodiment, the branched chain amino acid transporter gene has at least about 90% identity with the sequence of SEQ ID NO:10. Accordingly, in one embodiment, the branched chain amino acid transporter gene has at least about 95% identity with the sequence of SEQ ID NO:10. Accordingly, in one embodiment, the branched chain amino acid transporter gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:10. In another embodiment, the branched chain amino acid transporter gene comprises the sequence of SEQ ID NO:10. In yet another embodiment the branched chain amino acid transporter gene consists of the sequence of SEQ ID NO:10.

In some embodiments, the branched chain amino acid transporter is encoded by an branched chain amino acid transporter gene derived from a bacterial genus or species, including but not limited to, Escherichia coli. In some embodiments, the bacterial species is Escherichia coli. In some embodiments, the bacterial species is Escherichia coli strain Nissle.

Assays for testing the activity of a branched chain amino acid transporter, a functional variant of a branched chain amino acid transporter, or a functional fragment of a branched chain amino acid transporter are well known to one of ordinary skill in the art. For example, import of an amino acid may be determined using the methods as described in Hancy et al., J. Bact., 174 (1): 108-15, 1992; Rahmanian et al., J. Bact., 116 (3): 1258-66, 1973; and Ribardo and Hendrixson, J. Bact., 173 (22): 6233-43, 2011, the entire contents of each of which are expressly incorporated by reference herein.

In one embodiment, when the branched chain amino acid transporter is expressed in the recombinant bacterial cells described herein, the bacterial cells import 10% more branched chain amino acid into the bacterial cell when the transporter is expressed than unmodified bacteria of the same bacterial subtype under the same conditions. In another embodiment, when the transporter of branched chain amino acids is expressed in the recombinant bacterial cells described herein, the bacterial cells import 20%, 30%, 40%, 50%, 60%, 70%, 80%, 90% or 100% more branched chain amino acids into the bacterial cell when the transporter is expressed than unmodified bacteria of the same bacterial subtype under the same conditions. In yet another embodiment, when the branched chain amino acid transporter is expressed in the recombinant bacterial cells described herein, the bacterial cell imports two-fold more branched chain amino acids into the cell when the transporter is expressed than unmodified bacteria of the same bacterial subtype under the same conditions. In yet another embodiment, when the branched chain amino acid transporter is expressed in the recombinant bacterial cell described herein, the bacterial cell import three-fold, four-fold, five-fold, six-fold, seven-fold, eight-fold, nine-fold, or ten-fold, fifteen-fold, twenty-fold, thirty-fold, forty-fold, or fifty-fold, more branched chain amino acid into the cell when the transporter is expressed than unmodified bacteria of the same bacterial subtype under the same conditions.

In some embodiment, the recombinant bacterial cell comprising a heterologous gene encoding a branched chain amino acid transporter may be used to treat a disease, condition, and/or symptom associated with the catabolism of a branched chain amino acid. In some embodiments, disclosed herein are methods for reducing, ameliorating, or eliminating one or more symptom(s) associated with these diseases or disorders. In one embodiment, the disorder involving the catabolism of a branched chain amino acid is a metabolic disorder involving the abnormal catabolism of a branched chain amino acid. Metabolic diseases associated with abnormal catabolism of a branched chain amino acid include maple syrup urine disease (MSUD), isovaleric acidemia, propionic acidemia, methylmalonic acidemia, and diabetes ketoacidosis. In one embodiment, the disease associated with abnormal catabolism of a branched chain amino acid is isovaleric acidemia. In one embodiment, the disease associated with abnormal catabolism of a branched chain amino acid is propionic acidemia. In one embodiment, the disease associated with abnormal catabolism of a branched chain amino acid is methylmalonic acidemia. In another embodiment, the disease associated with abnormal catabolism of a branched chain amino acid is diabetes.

### 2. Arginine Transporters

In one embodiment, the amino acid transporter is an arginine transporter. Arginine transporters may be expressed or modified in the recombinant bacteria described herein in order to enhance arginine transport into the cell. Specifically, when the arginine transporter is expressed in the recombinant bacterial cells described herein, the bacterial cells import more arginine into the cell when the arginine transporter is expressed than unmodified bacteria of the same bacterial subtype under the same conditions. Thus, the genetically engineered bacteria comprising a heterologous gene encoding an arginine transporter which may be used to import arginine into the bacteria.

The uptake of arginine into bacterial cells is mediated by proteins well known to those of skill in the art. For example, three different arginine transport systems have been characterized in several bacteria: the arginine-specific system encoded by the artPIQM operon and the art gene (see, e.g., Wissenbach et al. (1993) J. Bacteriol. 175 (11): 3687-8); the basic amino acid uptake system, known as LAO (lysine, arginine, ornithine) (see, e.g., Rosin et al. (1971) J. Biol. Chem. 246:3653-62); and the AO (arginine, ornithine) system (see, e.g., Celis (1977) J. Bacteriol. 130:1234-43). Transport by the arginine-specific system is mediated by several proteins encoded by the two transcriptional units, the artPIQM operon and the artJ gene. In this system, ArtP (encoded by artP) is an ATPase, ArtQ and ArtM (encoded by artQ and artM, respectively) are transmembrane proteins, and ArtI and ArtJ (encoded by artI and artJ, respectively) are arginine-binding periplasmic proteins. This system has been well characterized in Escherichia coli (see, e.g., Wissenbach U. (1995) Mol. Microbiol. 17 (4): 675-86; Wissenbach et al. (1993) J. Bacteriol. 175 (11): 3687-88). In addition, bacterial systems that are homologous and orthologous of the E. coli arginine-specific system have been characterized in other bacterial species, including, for example, Haemophilus influenzae (see, e.g., Mironov et al. (1999) Nucleic Acids Res. 27 (14): 2981-9). The second arginine transport system, the basic amino acid LAO system, consists of the periplasmic LAO protein (also referred to herein as ArgT; encoded by argT), which binds lysine, arginine and ornithine, and the membranous and membrane-associated proteins of the histidine permease (Q M P complex), encoded by the hisJQMP operon, resulting in the uptake of arginine (see, e.g., Oh et al. (1994) J. Biol. Chem. 269 (42): 26323-30). Members of the basic amino acid LAO system have been well characterized in Escherichia coli and Salmonella enterica. Finally, the third arginine transport system, the AO system, consists of the binding protein AbpS (encoded by abpS) and the ATP hydrolase ArgK (encoded by argK) which mediate the ATP-dependent uptake of arginine (see, e.g., Celis et al. (1998) J. Bacteriol. 180 (18): 4828-33).

In one embodiment, the at least one gene encoding an arginine transporter is the artJ gene. In one embodiment, the at least one gene encoding an arginine transporter is the artPIQM operon. In one embodiment, the at least one gene encoding an arginine transporter is the artP gene. In one embodiment, the at least one gene encoding an arginine transporter is the artI gene. In one embodiment, the at least one gene encoding an arginine transporter is the artQ gene. In one embodiment, the at least one gene encoding an arginine transporter is the artM gene. In one embodiment, the at least one gene encoding an arginine transporter is the argT gene. In one embodiment, the at least one gene encoding an arginine transporter is the hisJQMP operon. In one embodiment, the at least one gene encoding an arginine transporter is the hisJ gene. In one embodiment, the at least one gene encoding an arginine transporter is the hisQ gene. In one embodiment, the at least one gene encoding an arginine transporter is the hisM gene. In one embodiment, the at least one gene encoding an arginine transporter is the hisP gene. In one embodiment, the at least one gene encoding an arginine transporter is the abpS gene. In one embodiment, the at least one gene encoding an arginine transporter is the argK gene. In another embodiment, the at least one gene encoding an arginine transporter comprises the artPIQM operon and the artJ gene. In another embodiment, the at least one gene encoding an arginine transporter comprises the hisJQMP operon and the argT gene. In yet another embodiment, the at least one gene encoding an arginine transporter comprises the abpS gene and the argK gene.

In one embodiment, the argT gene has at least about 80% identity with the sequence of SEQ ID NO:13. Accordingly, in one embodiment, the argT gene has at least about 90% identity with the sequence of SEQ ID NO: 13. Accordingly, in one embodiment, the argT gene has at least about 95% identity with the sequence of SEQ ID NO: 13. Accordingly, in one embodiment, the argT gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 13. In another embodiment, the argT gene comprises the sequence of SEQ ID NO:13. In yet another embodiment the argT gene consists of the sequence of SEQ ID NO:13.

In one embodiment, the artP gene has at least about 80% identity with the sequence of SEQ ID NO:14. Accordingly, in one embodiment, the artP gene has at least about 90% identity with the sequence of SEQ ID NO:14. Accordingly, in one embodiment, the artP gene has at least about 95% identity with the sequence of SEQ ID NO:14. Accordingly, in one embodiment, the artP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 14. In another embodiment, the artP gene comprises the sequence of SEQ ID NO: 14. In yet another embodiment the artP gene consists of the sequence of SEQ ID NO: 14.

In one embodiment, the artI gene has at least about 80% identity with the sequence of SEQ ID NO: 15. Accordingly, in one embodiment, the artI gene has at least about 90% identity with the sequence of SEQ ID NO: 15. Accordingly, in one embodiment, the artI gene has at least about 95% identity with the sequence of SEQ ID NO:15. Accordingly, in one embodiment, the artI gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 15. In another embodiment, the art gene comprises the sequence of SEQ ID NO:15. In yet another embodiment the art gene consists of the sequence of SEQ ID NO: 15.

In one embodiment, the artQ gene has at least about 80% identity with the sequence of SEQ ID NO: 16. Accordingly, in one embodiment, the artQ gene has at least about 90% identity with the sequence of SEQ ID NO: 16. Accordingly, in one embodiment, the artQ gene has at least about 95% identity with the sequence of SEQ ID NO: 16. Accordingly, in one embodiment, the artQ gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 16. In another embodiment, the artQ gene comprises the sequence of SEQ ID NO:16. In yet another embodiment the artQ gene consists of the sequence of SEQ ID NO:16.

In one embodiment, the artM gene has at least about 80% identity with the sequence of SEQ ID NO:17. Accordingly, in one embodiment, the artM gene has at least about 90% identity with the sequence of SEQ ID NO: 17. Accordingly, in one embodiment, the artM gene has at least about 95% identity with the sequence of SEQ ID NO:17. Accordingly, in one embodiment, the artM gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:17. In another embodiment, the artM gene comprises the sequence of SEQ ID NO: 17. In yet another embodiment the artM gene consists of the sequence of SEQ ID NO:17.

In one embodiment, the artJ gene has at least about 80% identity with the sequence of SEQ ID NO: 18. Accordingly, in one embodiment, the art gene has at least about 90% identity with the sequence of SEQ ID NO: 18. Accordingly, in one embodiment, the artJ gene has at least about 95% identity with the sequence of SEQ ID NO: 18. Accordingly, in one embodiment, the art gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 18. In another embodiment, the artJ gene comprises the sequence of SEQ ID NO:18. In yet another embodiment the artJ gene consists of the sequence of SEQ ID NO: 18.

In some embodiments, the arginine transporter is encoded by an arginine transporter gene derived from a bacterial genus or species, including but not limited to, Escherichia, Haemophilus, Salmonella, Escherichia coli, Haemophilus influenza, Salmonella enterica, or Salmonella typhimurium. In some embodiments, the bacterial species is Escherichia coli. In some embodiments, the bacterial species is Escherichia coli strain Nissle.

Assays for testing the activity of an arginine transporter, a functional variant of an arginine transporter, or a functional fragment of arginine transporter are well known to one of ordinary skill in the art. For example, import of arginine may be determined using the methods as described in Sakanaka et al (2015) J. Biol. Chem. 290 (35): 21185-98, the entire contents of each of which are expressly incorporated by reference herein.

In one embodiment, when the arginine transporter is expressed in the recombinant bacterial cells described herein, the bacterial cells import 10% more arginine into the bacterial cell when the arginine transporter is expressed than unmodified bacteria of the same bacterial subtype under the same conditions. In another embodiment, when the arginine transporter is expressed in the recombinant bacterial cells described herein, the bacterial cells import 20%, 30%, 40%, 50%, 60%, 70%, 80%, 90% or 100% more arginine into the bacterial cell when the arginine transporter is expressed than unmodified bacteria of the same bacterial subtype under the same conditions. In yet another embodiment, when the arginine transporter is expressed in the recombinant bacterial cells described herein, the bacterial cells import two-fold more arginine into the cell when the transporter is expressed than unmodified bacteria of the same bacterial subtype under the same conditions. In yet another embodiment, when the arginine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more arginine into the cell when the transporter is expressed than unmodified bacteria of the same bacterial subtype under the same conditions.

### 3. Lysine Transporters

In one embodiment, the amino acid transporter is a lysine transporter. Lysine transporters may be expressed or modified in the recombinant bacteria described herein in order to enhance lysine transport into the cell. Specifically, when the transporter of lysine is expressed in the recombinant bacterial cells described herein, the bacterial cells import more lysine into the cell when the lysine transporter is expressed than unmodified bacteria of the same bacterial subtype under the same conditions. Thus, the genetically engineered bacteria comprising a heterologous gene encoding a lysine transporter which may be used to import lysine into the bacteria.

The uptake of lysine into bacterial cells is mediated by proteins well known to those of skill in the art. For example, LysP is a lysine-specific permease originally identified in E. coli, that has now been further characterized in other bacterial species (Steffes et al. (1992) J. Bacteriol. 174:3242-9; Trip et al. (2013) J. Bacteriol. 195 (2): 340-50; Nji et al. (2014) Acta Crystallogr. F Struct. Biol. Commun. 70 (Pt 10): 1362-7). Another lysine transporter, YsvH, has been described in Bacillus, having similarities to the lysine permease LysI of Corynebacterium glutamicum (Rodionov et al. (2003) Nucleic Acids Res. 31 (23): 6748-57).

In one embodiment, the at least one gene encoding a lysine transporter is the lysP gene. In one embodiment, the bacterial cell described herein has been genetically engineered to comprise at least one heterologous lysP gene. In one embodiment, the at least one gene encoding a lysine transporter is the Escherichia coli lysP gene. In one embodiment, the at least one gene encoding a lysine transporter is the Lactococcus lactis lysP gene. In one embodiment, the at least one gene encoding a lysine transporter is the Pseudomonas aeruginosa lysP gene. In one embodiment, the at least one gene encoding a lysine transporter is the Klebsiella pneumoniae lysP gene.

In one embodiment, the lysP gene has at least about 80% identity with the sequence of SEQ ID NO:26. Accordingly, in one embodiment, the lysP gene has at least about 90% identity with the sequence of SEQ ID NO:26. Accordingly, in one embodiment, the lysP gene has at least about 95% identity with the sequence of SEQ ID NO:26. Accordingly, in one embodiment, the lysP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 26. In another embodiment, the lysP gene comprises the sequence of SEQ ID NO:26. In yet another embodiment the lysP gene consists of the sequence of SEQ ID NO:26.

In one embodiment, the at least one gene encoding a lysine transporter is the ysvH gene. In one embodiment, the bacterial cell described herein has been genetically engineered to comprise at least one heterologous ysvH gene. In one embodiment, the at least one gene encoding a lysine transporter is the Bacillus subtilis ysvH gene. In one embodiment, the at least one gene encoding a lysine transporter is the Bacillus cereus ysvH gene. In one embodiment, the at least one gene encoding a lysine transporter is the Bacillus stearothermophilus ysvH gene.

In one embodiment, the at least one gene encoding a lysine transporter is the Corynebacterium glutamicum (see, e.g., Scep-Feldhaus et al. (1991) Mol. Microbiol. 5 (12): 2995-3005, the entire contents of which are incorporated herein by reference).

In one embodiment, the ysvH gene has at least about 80% identity with the sequence of SEQ ID NO:25. Accordingly, in one embodiment, the ysvH gene has at least about 90% identity with the sequence of SEQ ID NO:25. Accordingly, in one embodiment, the ysvH gene has at least about 95% identity with the sequence of SEQ ID NO:25. Accordingly, in one embodiment, the ysvH gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:25. In another embodiment, the ysvH gene comprises the sequence of SEQ ID NO: 25. In yet another embodiment the ysvH gene consists of the sequence of SEQ ID NO:25.

In some embodiments, the transporter of lysine is encoded by a lysine transporter gene derived from a bacterial genus or species, including but not limited to, Bacillus subtilis, Bacillus cereus, Bacillus stearothermophilus, Corynebacterium glutamicum, Escherichia coli, Lactococcus lactis, Pseudomonas aeruginosa, and Klebsiella pneumoniae. In some embodiments, the bacterial species is Escherichia coli. In some embodiments, the bacterial species is Escherichia coli strain Nissle.

Assays for testing the activity of a lysine transporter, a functional variant of a lysine transporter, or a functional fragment of a lysine transporter are well known to one of ordinary skill in the art. For example, import of lysine may be determined using the methods as described in Steffes et al. (1992) J. Bacteriol. 174:3242-9, the entire contents of each of which are expressly incorporated by reference herein.

In one embodiment, when the lysine transporter is expressed in the recombinant bacterial cells described herein, the bacterial cells import 10% more lysine into the bacterial cell when the lysine transporter is expressed than unmodified bacteria of the same bacterial subtype under the same conditions. In another embodiment, when the lysine transporter is expressed in the recombinant bacterial cells described herein, the bacterial cells import 20%, 30%, 40%, 50%, 60%, 70%, 80%, 90% or 100% more lysine into the bacterial cell when the transporter is expressed than unmodified bacteria of the same bacterial subtype under the same conditions. In yet another embodiment, when the lysine transporter is expressed in the recombinant bacterial cells described herein, the bacterial cells import two-fold more lysine into the cell when the transporter is expressed than unmodified bacteria of the same bacterial subtype under the same conditions. In yet another embodiment, when the transporter of lysine is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more lysine into the cell when the transporter is expressed than unmodified bacteria of the same bacterial subtype under the same conditions.

### 4. Asparagine Transporters

In one embodiment, the amino acid transporter is an asparagine transporter. Asparagine transporters may be expressed or modified in the recombinant bacteria described herein in order to enhance asparagine transport into the cell. Specifically, when the asparagine transporter is expressed in the recombinant bacterial cells described herein, the bacterial cells import more asparagine into the cell when the asparagine transporter is expressed than unmodified bacteria of the same bacterial subtype under the same conditions. Thus, the genetically engineered bacteria comprising a heterologous gene encoding an asparagine transporter which may be used to import asparagine into the bacteria.

The uptake of asparagine into bacterial cells is mediated by proteins well known to those of skill in the art. For example, two distinct systems for asparagine uptake, distinguishable on the basis of their specificity for asparagine have been identified in E. coli (see, e.g., Willis and Woolfolk (1975) J. Bacteriol. 123:937-945). The bacterial gene ansP encodes an asparagine permease responsible for asparagine uptake in many bacteria (see, e.g., Jennings et al. (1995) Microbiology 141:141-6; Ortuño-Olea and Durán-Vargas (2000) FEMS Microbiol. Lett. 189 (2): 177-82; Barel et al. (2015) Front. Cell. Infect. Microbiol. 5:9; and Gouzy et al. (2014) PLoS Pathog. 10 (2): €1003928).

In one embodiment, the at least one gene encoding an asparagine transporter is the ansP gene. In one embodiment, the bacterial cell described herein has been genetically engineered to comprise at least one heterologous ansP gene. In one embodiment, the at least one gene encoding an asparagine transporter is the Escherichia coli ansP gene. In one embodiment, the at least one gene encoding an asparagine transporter is the Francisella tularensis ansP gene. In one embodiment, the at least one gene encoding an asparagine transporter is the Mycobacterium bovis ansP2 gene. In one embodiment, the at least one gene encoding an asparagine transporter is the Salmonella enterica ansP gene. In one embodiment, the at least one gene encoding an asparagine transporter is the Yersinia pestis ansP gene.

In one embodiment, the ansP2 gene has at least about 80% identity with the sequence of SEQ ID NO:29. Accordingly, in one embodiment, the ansP2 gene has at least about 90% identity with the sequence of SEQ ID NO:29. Accordingly, in one embodiment, the ansP2 gene has at least about 95% identity with the sequence of SEQ ID NO:29. Accordingly, in one embodiment, the ansP2 gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:29. In another embodiment, the ansP2 gene comprises the sequence of SEQ ID NO: 29. In yet another embodiment the ansP2 gene consists of the sequence of SEQ ID NO:29.

In some embodiments, the asparagine transporter is encoded by an asparagine transporter gene derived from a bacterial genus or species, including but not limited to, Escherichia, Francisella, Mycobacterium, Salmonella, Yersinia, Escherichia coli, Francisella tularensis, Mycobacterium tuberculosis, Salmonella enterica, or Yersinia pestis. In some embodiments, the bacterial species is Escherichia coli. In some embodiments, the bacterial species is Escherichia coli strain Nissle.

Assays for testing the activity of an asparagine transporter, a functional variant of an asparagine transporter, or a functional fragment of asparagine transporter are well known to one of ordinary skill in the art. For example, import of asparagine may be determined using the methods as described in Jennings et al. (1995) Microbiology 141:141-6, the entire contents of each of which are expressly incorporated by reference herein.

In one embodiment, when the transporter of an asparagine is expressed in the recombinant bacterial cells described herein, the bacterial cells import 10% more asparagine into the bacterial cell when the transporter is expressed than unmodified bacteria of the same bacterial subtype under the same conditions. In another embodiment, when the asparagine transporter is expressed in the recombinant bacterial cells described herein, the bacterial cells import 20%, 30%, 40%, 50%, 60%, 70%, 80%, 90% or 100% more asparagine into the bacterial cell when the transporter is expressed than unmodified bacteria of the same bacterial subtype under the same conditions. In yet another embodiment, when the asparagine transporter is expressed in the recombinant bacterial cells described herein, the bacterial cells import two-fold more asparagine into the cell when the transporter is expressed than unmodified bacteria of the same bacterial subtype under the same conditions. In yet another embodiment, when the asparagine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more asparagine into the cell when the transporter is expressed than unmodified bacteria of the same bacterial subtype under the same conditions.

### 5. Serine Transporters

In one embodiment, the amino acid transporter is a serine transporter. Serine transporters may be expressed or modified in the recombinant bacteria described herein in order to enhance serine transport into the cell. Specifically, when the serine transporter is expressed in the recombinant bacterial cells described herein, the bacterial cells import more serine into the cell when the transporter is expressed than unmodified bacteria of the same bacterial subtype under the same conditions. Thus, the genetically engineered bacteria comprising a heterologous gene encoding a serine transporter which may be used to import serine into the bacteria.

The uptake of serine into bacterial cells is mediated by proteins well known to those of skill in the art. For example, SdaC (encoded by the sdaC gene; also known as DcrA) is an inner membrane threonine-insensitive serine transporter that was originally identified in Escherichia coli (Shao et al. (1994) Eur. J. Biochem. 222:901-7). Additional serine transporters that have been identified include the Na⁺/serine symporter, SstT (encoded by the sstT gene), the leucine-isoleucine-valine transporter LIV-1, which transports serine slowly, and the H⁺/serine-threonine symporter TdcC (encoded by the tdcC gene) (see, e.g., Ogawa et al. (1998) J. Bacteriol. 180:6749-52; Ogawa et al. (1997) J. Biochem. 122 (6): 1241-5).

In one embodiment, the at least one gene encoding a serine transporter is the sdaC gene. In one embodiment, the bacterial cell described herein has been genetically engineered to comprise at least one heterologous sdaC gene. In one embodiment, the at least one gene encoding a serine transporter is the Escherichia coli sdaC gene. In one embodiment, the at least one gene encoding a serine transporter is the Campylobacter jejuni sdaC gene.

In one embodiment, the sdaC gene has at least about 80% identity with the sequence of SEQ ID NO:35. Accordingly, in one embodiment, the sdaC gene has at least about 90% identity with the sequence of SEQ ID NO:35. Accordingly, in one embodiment, the sdaC gene has at least about 95% identity with the sequence of SEQ ID NO:35. Accordingly, in one embodiment, the sdaC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:35. In another embodiment, the sdaC gene comprises the sequence of SEQ ID NO: 35. In yet another embodiment the sdaC gene consists of the sequence of SEQ ID NO:35.

In one embodiment, the at least one gene encoding a serine transporter is the sstT gene. In one embodiment, the bacterial cell described herein has been genetically engineered to comprise at least one heterologous sstT gene. In one embodiment, the at least one gene encoding a serine transporter is the Escherichia coli sstT gene.

In one embodiment, the at least one gene encoding a serine transporter is the tdcC gene. In one embodiment, the bacterial cell described herein has been genetically engineered to comprise at least one heterologous tdcC gene. In one embodiment, the at least one gene encoding a serine transporter is the Escherichia coli tdcC gene.

In some embodiments, the serine transporter is encoded by a serine transporter gene derived from a bacterial genus or species, including but not limited to, Campylobacter, Campylobacter jejuni, Escherichia, and Escherichia coli In some embodiments, the bacterial species is Escherichia coli. In some embodiments, the bacterial species is Escherichia coli strain Nissle.

Assays for testing the activity of a serine transporter, a functional variant of a serine transporter, or a functional fragment of transporter of serine are well known to one of ordinary skill in the art. For example, import of serine may be determined using the methods as described in Hama et al. (1987) Biochim. Biophys. Acta 905:231-9, the entire contents of each of which are expressly incorporated by reference herein.

In one embodiment, when the transporter of a serine is expressed in the recombinant bacterial cells described herein, the bacterial cells import 10% more serine into the bacterial cell when the transporter is expressed than unmodified bacteria of the same bacterial subtype under the same conditions. In another embodiment, when the serine transporter is expressed in the recombinant bacterial cells described herein, the bacterial cells import 20%, 30%, 40%, 50%, 60%, 70%, 80%, 90% or 100% more serine into the bacterial cell when the transporter is expressed than unmodified bacteria of the same bacterial subtype under the same conditions. In yet another embodiment, when the serine transporter is expressed in the recombinant bacterial cells described herein, the bacterial cells import two-fold more serine into the cell when the transporter is expressed than unmodified bacteria of the same bacterial subtype under the same conditions. In yet another embodiment, when the serine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more serine into the cell when the transporter is expressed than unmodified bacteria of the same bacterial subtype under the same conditions.

### 6. Glutamine Transporters

In one embodiment, the amino acid transporter is a glutamine transporter. Glutamine transporters may be expressed or modified in the recombinant bacteria described herein in order to enhance glutamine transport into the cell. Specifically, when the glutamine transporter is expressed in the recombinant bacterial cells described herein, the bacterial cells import more glutamine into the cell when the transporter is expressed than unmodified bacteria of the same bacterial subtype under the same conditions. Thus, the genetically engineered bacteria comprising a heterologous gene encoding a glutamine transporter which may be used to import glutamine into the bacteria.

The uptake of glutamine into bacterial cells is mediated by proteins well known to those of skill in the art. For example, a glutamine permease glnHPQ operon has been identified in Escherichia coli (Nohno et al., Mol. Gen. Genet. 205 (2): 260-269, 1986).

In one embodiment, the at least one gene encoding a glutamine transporter is the glnHPQ operon. In one embodiment, the bacterial cell described herein has been genetically engineered to comprise at least one heterologous gene from the glnHPQ operon. In one embodiment, the bacterial cell described herein has been genetically engineered to comprise at least one heterologous glnH gene. In one embodiment, the bacterial cell described herein has been genetically engineered to comprise at least one heterologous glnP gene. In one embodiment, the bacterial cell described herein has been genetically engineered to comprise at least one heterologous glnQ gene.

In one embodiment, the glnHPQ operon has at least about 80% identity with the sequence of SEQ ID NO:41. Accordingly, in one embodiment, the glnHPQ operon has at least about 90% identity with the sequence of SEQ ID NO:41. Accordingly, in one embodiment, the glnHPQ operon has at least about 95% identity with the sequence of SEQ ID NO:41. Accordingly, in one embodiment, the glnHPQ operon has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:41. In another embodiment, the glnHPQ operon comprises the sequence of SEQ ID NO:41. In yet another embodiment the glnHPQ operon consists of the sequence of SEQ ID NO:41.

In one embodiment, the glnH gene has at least about 80% identity with the sequence of SEQ ID NO:42. Accordingly, in one embodiment, the glnH gene has at least about 90% identity with the sequence of SEQ ID NO:42. Accordingly, in one embodiment, the glnH gene has at least about 95% identity with the sequence of SEQ ID NO:42. Accordingly, in one embodiment, the glnH gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:42. In another embodiment, the glnH gene comprises the sequence of SEQ ID NO: 42. In yet another embodiment the glnH gene consists of the sequence of SEQ ID NO:42.

In one embodiment, the glnP gene has at least about 80% identity with the sequence of SEQ ID NO:43. Accordingly, in one embodiment, the glnP gene has at least about 90% identity with the sequence of SEQ ID NO:43. Accordingly, in one embodiment, the glnP gene has at least about 95% identity with the sequence of SEQ ID NO:43. Accordingly, in one embodiment, the glnP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 43. In another embodiment, the glnP gene comprises the sequence of SEQ ID NO:43. In yet another embodiment the glnP gene consists of the sequence of SEQ ID NO:43.

In one embodiment, the glnQ gene has at least about 80% identity with the sequence of SEQ ID NO:44. Accordingly, in one embodiment, the glnQ gene has at least about 90% identity with the sequence of SEQ ID NO:44. Accordingly, in one embodiment, the glnQ gene has at least about 95% identity with the sequence of SEQ ID NO:44. Accordingly, in one embodiment, the glnQ gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:44. In another embodiment, the glnQ gene comprises the sequence of SEQ ID NO: 44. In yet another embodiment the glnQ gene consists of the sequence of SEQ ID NO:44.

In some embodiments, the glutamine transporter is encoded by a glutamine transporter gene derived from a bacterial genus or species, including but not limited to, Escherichia coli. In some embodiments, the bacterial species is Escherichia coli strain Nissle.

Assays for testing the activity of a glutamine transporter, a functional variant of a glutamine transporter, or a functional fragment of transporter of glutamine are well known to one of ordinary skill in the art. For example, import of glutamine may be determined using the methods as described in Nohno et al., Mol. Gen. Genet., 205 (2): 260-269, 1986, the entire contents of which are expressly incorporated by reference herein.

In one embodiment, when the glutamine transporter is expressed in the recombinant bacterial cells described herein, the bacterial cells import 10% more glutamine into the bacterial cell when the transporter is expressed than unmodified bacteria of the same bacterial subtype under the same conditions. In another embodiment, when the glutamine transporter is expressed in the recombinant bacterial cells described herein, the bacterial cells import 20%, 30%, 40%, 50%, 60%, 70%, 80%, 90% or 100% more glutamine into the bacterial cell when the transporter is expressed than unmodified bacteria of the same bacterial subtype under the same conditions. In yet another embodiment, when the glutamine transporter is expressed in the recombinant bacterial cells described herein, the bacterial cells import two-fold more glutamine into the cell when the transporter is expressed than unmodified bacteria of the same bacterial subtype under the same conditions. In yet another embodiment, when the glutamine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more glutamine into the cell when the transporter is expressed than unmodified bacteria of the same bacterial subtype under the same conditions.

### 7. Tryptophan Transporters

In one embodiment, the amino acid transporter is a tryptophan transporter. Tryptophan transporters may be expressed or modified in the recombinant bacteria described herein in order to enhance tryptophan transport into the cell. Specifically, when the tryptophan transporter is expressed in the recombinant bacterial cells described herein, the bacterial cells import more tryptophan into the cell when the transporter is expressed than unmodified bacteria of the same bacterial subtype under the same conditions. Thus, the genetically engineered bacteria comprising a heterologous gene encoding a tryptophan transporter which may be used to import tryptophan into the bacteria.

The uptake of tryptophan into bacterial cells is mediated by proteins well known to those of skill in the art. For example, three different tryptophan transporters, distinguishable on the basis of their affinity for tryptophan have been identified in E. coli (see, e.g., Yanofsky et al. (1991) J. Bacteriol. 173:6009-17). The bacterial genes mtr, aroP, and tnaB encode tryptophan permeases responsible for tryptophan uptake in bacteria. High affinity permease, Mtr, is negatively regulated by the trp repressor and positively regulated by the TyR product (see, e.g., Yanofsky et al. (1991) J. Bacteriol. 173:6009-17 and Heatwole et al. (1991) J. Bacteriol. 173:3601-04), while AroP is negatively regulated by the tyR product (Chye et al. (1987) J. Bacteriol. 169:386-93).

In one embodiment, the at least one gene encoding a tryptophan transporter is a gene selected from the group consisting of mtr, aroP and tnaB. In one embodiment, the bacterial cell described herein has been genetically engineered to comprise at least one heterologous gene selected from the group consisting of mtr, aroP and tnaB. In one embodiment, the at least one gene encoding a tryptophan transporter is the Escherichia coli mtr gene. In one embodiment, the at least one gene encoding a tryptophan transporter is the Escherichia coli aroP gene. In one embodiment, the at least one gene encoding a tryptophan transporter is the Escherichia coli tnaB gene.

In one embodiment, the mtr gene has at least about 80% identity with the sequence of SEQ ID NO:46. Accordingly, in one embodiment, the mtr gene has at least about 90% identity with the sequence of SEQ ID NO:46. Accordingly, in one embodiment, the mtr gene has at least about 95% identity with the sequence of SEQ ID NO:46. Accordingly, in one embodiment, the mtr gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 46. In another embodiment, the mtr gene comprises the sequence of SEQ ID NO:46. In yet another embodiment the mtr gene consists of the sequence of SEQ ID NO:46.

In one embodiment, the tnaB gene has at least about 80% identity with the sequence of SEQ ID NO:47. Accordingly, in one embodiment, the tnaB gene has at least about 90% identity with the sequence of SEQ ID NO:47. Accordingly, in one embodiment, the tnaB gene has at least about 95% identity with the sequence of SEQ ID NO:47. Accordingly, in one embodiment, the tnaB gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 47. In another embodiment, the tnaB gene comprises the sequence of SEQ ID NO:47. In yet another embodiment the tnaB gene consists of the sequence of SEQ ID NO:47.

In one embodiment, the aroP gene has at least about 80% identity with the sequence of SEQ ID NO:48. Accordingly, in one embodiment, the aroP gene has at least about 90% identity with the sequence of SEQ ID NO:48. Accordingly, in one embodiment, the aroP gene has at least about 95% identity with the sequence of SEQ ID NO:48. Accordingly, in one embodiment, the aroP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:48. In another embodiment, the aroP gene comprises the sequence of SEQ ID NO: 48. In yet another embodiment the aroP gene consists of the sequence of SEQ ID NO:48.

In some embodiments, the tryptophan transporter is encoded by a tryptophan transporter gene derived from a bacterial genus or species, including but not limited to, Escherichia, Corynebacterium, Escherichia coli, Saccharomyces cerevisiae or

Corynebacterium glutamicum. In some embodiments, the bacterial species is Escherichia coli. In some embodiments, the bacterial species is Escherichia coli strain Nissle.

Assays for testing the activity of a tryptophan transporter, a functional variant of a tryptophan transporter, or a functional fragment of transporter of tryptophan are well known to one of ordinary skill in the art. For example, import of tryptophan may be determined using the methods as described in Shang et al. (2013) J. Bacteriol. 195:5334-42, the entire contents of each of which are expressly incorporated by reference herein.

In one embodiment, when the tryptophan transporter is expressed in the recombinant bacterial cells described herein, the bacterial cells import 10% more tryptophan into the bacterial cell when the transporter is expressed than unmodified bacteria of the same bacterial subtype under the same conditions. In another embodiment, when the tryptophan transporter is expressed in the recombinant bacterial cells described herein, the bacterial cells import 20%, 30%, 40%, 50%, 60%, 70%, 80%, 90% or 100% more tryptophan into the bacterial cell when the transporter is expressed than unmodified bacteria of the same bacterial subtype under the same conditions. In yet another embodiment, when the tryptophan transporter is expressed in the recombinant bacterial cells described herein, the bacterial cells import two-fold more tryptophan into the cell when the transporter is expressed than unmodified bacteria of the same bacterial subtype under the same conditions. In yet another embodiment, when the tryptophan transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more tryptophan into the cell when the transporter is expressed than unmodified bacteria of the same bacterial subtype under the same conditions.

### 8. Methionine Transporters

In one embodiment, the amino acid transporter is a methionine transporter. Methionine transporters may be expressed or modified in the recombinant bacteria described herein in order to enhance methionine transport into the cell. Specifically, when the methionine transporter is expressed in the recombinant bacterial cells described herein, the bacterial cells import more methionine into the cell when the transporter is expressed than unmodified bacteria of the same bacterial subtype under the same conditions. Thus, the genetically engineered bacteria comprising a heterologous gene encoding a methionine transporter which may be used to import methionine into the bacteria.

The uptake of methionine into bacterial cells is mediated by proteins well known to those of skill in the art. For example, a methionine transporter operon has been identified in Corynebacterium glutamicum (Trotschel et al., J. Bacteriology, 187 (11): 3786-3794, 2005). In addition, the high affinity MetD ABC transporter system has been characterized in Escherichia coli (Kadaba et al. (2008) Science 5886:250-253; Kadner and Watson (1974) J. Bacteriol. 119:401-9). The MetD transporter system is capable of mediating the translocation of several substrates across the bacterial membrane, including methionine. The metD system of Escherichia coli consists of MetN (encoded by metN), which comprises the ATPase domain, MetI (encoded by metI), which comprises the transmembrane domain, and MetQ (encoded by metQ), the cognate binding protein which is located in the periplasm. Orthologues of the genes encoding the E. coli metD transporter system have been identified in multiple organisms including, e.g., Yersinia pestis, Vibrio cholerae, Pasteurella multocida, Haemophilus influenza, Agrobacterium tumefaciens, Sinorhizobium meliloti, Brucella meliloti, and Mesorhizobium loti (Merlin et al. (2002) J. Bacteriol. 184:5513-7).

In one embodiment, the at least one gene encoding a methionine transporter is a metP gene, a metN gene, a metI gene, or a metQ gene from Corynebacterium glutamicum, Escherichia coli, and Bacillus subtilis (Trotschel et al., J. Bacteriology, 187 (11): 3786-3794, 2005).

In one embodiment, the metP gene has at least about 80% identity with the sequence of SEQ ID NO:59. Accordingly, in one embodiment, the metP gene has at least about 90% identity with the sequence of SEQ ID NO:59. Accordingly, in one embodiment, the metP gene has at least about 95% identity with the sequence of SEQ ID NO:59. Accordingly, in one embodiment, the metP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:59. In another embodiment, the metP gene comprises the sequence of SEQ ID NO: 59. In yet another embodiment the metP gene consists of the sequence of SEQ ID NO:59.

In one embodiment, the metN gene has at least about 80% identity with the sequence of SEQ ID NO:60. Accordingly, in one embodiment, the metN gene has at least about 90% identity with the sequence of SEQ ID NO:60. Accordingly, in one embodiment, the metN gene has at least about 95% identity with the sequence of SEQ ID NO:60. Accordingly, in one embodiment, the metN gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:60. In another embodiment, the metN gene comprises the sequence of SEQ ID NO: 60. In yet another embodiment the metN gene consists of the sequence of SEQ ID NO:60.

In one embodiment, the metI gene has at least about 80% identity with the sequence of SEQ ID NO:61. Accordingly, in one embodiment, the metI gene has at least about 90% identity with the sequence of SEQ ID NO:61. Accordingly, in one embodiment, the metI gene has at least about 95% identity with the sequence of SEQ ID NO:61. Accordingly, in one embodiment, the metI gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 61. In another embodiment, the metI gene comprises the sequence of SEQ ID NO:61. In yet another embodiment the metI gene consists of the sequence of SEQ ID NO:61.

In one embodiment, the metQ gene has at least about 80% identity with the sequence of SEQ ID NO:62. Accordingly, in one embodiment, the metQ gene has at least about 90% identity with the sequence of SEQ ID NO:62. Accordingly, in one embodiment, the metQ gene has at least about 95% identity with the sequence of SEQ ID NO:62. Accordingly, in one embodiment, the metQ gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:62. In another embodiment, the metQ gene comprises the sequence of SEQ ID NO: 62. In yet another embodiment the metQ gene consists of the sequence of SEQ ID NO:62.

In some embodiments, the methionine transporter is encoded by a methionine transporter gene derived from a bacterial genus or species, including but not limited to, Corynebacterium glutamicum, Escherichia coli, and Bacillus subtilis. In some embodiments, the bacterial species is Escherichia coli strain Nissle.

Assays for testing the activity of a methionine transporter, a functional variant of a methionine transporter, or a functional fragment of a methionine transporter are well known to one of ordinary skill in the art. For example, import of methionine may be determined using the methods as described in Trotschel et al., J. Bacteriology, 187 (11): 3786-3794, 2005, the entire contents of which are expressly incorporated by reference herein.

In one embodiment, when the methionine transporter is expressed in the recombinant bacterial cells described herein, the bacterial cells import 10% more methionine into the bacterial cell when the transporter is expressed than unmodified bacteria of the same bacterial subtype under the same conditions. In another embodiment, when the methionine transporter is expressed in the recombinant bacterial cells described herein, the bacterial cells import 20%, 30%, 40%, 50%, 60%, 70%, 80%, 90% or 100% more methionine into the bacterial cell when the transporter is expressed than unmodified bacteria of the same bacterial subtype under the same conditions. In yet another embodiment, when the methionine transporter is expressed in the recombinant bacterial cells described herein, the bacterial cells import two-fold more methionine into the cell when the transporter is expressed than unmodified bacteria of the same bacterial subtype under the same conditions. In yet another embodiment, when the methionine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more methionine into the cell when the transporter is expressed than unmodified bacteria of the same bacterial subtype under the same conditions.

### 9. Threonine Transporters

In one embodiment, the amino acid transporter is a threonine transporter. Threonine transporters may be expressed or modified in the recombinant bacteria described herein in order to enhance threonine transport into the cell. Specifically, when the threonine transporter is expressed in the recombinant bacterial cells described herein, the bacterial cells import more threonine into the cell when the transporter is expressed than unmodified bacteria of the same bacterial subtype under the same conditions. Thus, the genetically engineered bacteria comprising a heterologous gene encoding a threonine transporter which may be used to import threonine into the bacteria.

The uptake of threonine into bacterial cells is mediated by proteins well known to those of skill in the art. For example, the threonine transporter TdcC has been identified (Wook Lee et al., Nature Chemical Biology, 8:536-546, 2012). Additional serine/threonine transporters have been identified and are disclosed in the serine section herein.

In one embodiment, the at least one gene encoding a threonine transporter is the tdcC gene. In one embodiment, the bacterial cell described herein has been genetically engineered to comprise at least one heterologous tdcC gene. In one embodiment, the at least one gene encoding a threonine transporter is the Escherichia coli tdcC gene. In one embodiment, the at least one gene encoding a threonine transporter is the Salmonella typhimurium tdcC gene.

In one embodiment, the tdcC gene has at least about 80% identity with the sequence of SEQ ID NO:69. Accordingly, in one embodiment, the tdcC gene has at least about 90% identity with the sequence of SEQ ID NO:69. Accordingly, in one embodiment, the tdcC gene has at least about 95% identity with the sequence of SEQ ID NO:69. Accordingly, in one embodiment, the tdcC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 69. In another embodiment, the tdcC gene comprises the sequence of SEQ ID NO:69. In yet another embodiment the tdcC gene consists of the sequence of SEQ ID NO:69.

In some embodiments, the threonine transporter is encoded by a threonine transporter gene derived from a bacterial genus or species, including but not limited to, Escherichia coli or Salmonella typhimurium. In some embodiments, the bacterial species is Escherichia coli. In some embodiments, the bacterial species is Escherichia coli strain Nissle.

Assays for testing the activity of a threonine transporter, a functional variant of a threonine transporter, or a functional fragment of transporter of threonine are well known to one of ordinary skill in the art. For example, import of threonine may be determined using the methods as described in Wook Lec et al. (2012) Nature Chemical Biology, 8:536-546, the entire contents of which are expressly incorporated by reference herein.

In one embodiment, when the threonine transporter is expressed in the recombinant bacterial cells described herein, the bacterial cells import 10% more threonine into the bacterial cell when the transporter is expressed than unmodified bacteria of the same bacterial subtype under the same conditions. In another embodiment, when the threonine transporter is expressed in the recombinant bacterial cells described herein, the bacterial cells import 20%, 30%, 40%, 50%, 60%, 70%, 80%, 90% or 100% more threonine into the bacterial cell when the transporter is expressed than unmodified bacteria of the same bacterial subtype under the same conditions. In yet another embodiment, when the threonine transporter is expressed in the recombinant bacterial cells described herein, the bacterial cells import two-fold more threonine into the cell when the transporter is expressed than unmodified bacteria of the same bacterial subtype under the same conditions. In yet another embodiment, when the threonine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more threonine into the cell when the transporter is expressed than unmodified bacteria of the same bacterial subtype under the same conditions.

### 10. Cysteine Transporters

In one embodiment, the amino acid transporter is a cysteine transporter. Cysteine transporters may be expressed or modified in the recombinant bacteria described herein in order to enhance cysteine transport into the cell. Specifically, when the cysteine transporter is expressed in the recombinant bacterial cells described herein, the bacterial cells import more cysteine into the cell when the transporter is expressed than unmodified bacteria of the same bacterial subtype under the same conditions. Thus, the genetically engineered bacteria comprising a heterologous gene encoding a cysteine transporter which may be used to import cysteine into the bacteria so that any gene encoding a cysteine catabolism enzyme expressed in the organism can catabolize the cysteine to treat a disease associated with cysteine, such as cancer.

The uptake of cysteine into bacterial cells is mediated by proteins well known to those of skill in the art.

In some embodiments, the cysteine transporter is encoded by a cysteine transporter gene derived from a bacterial genus or species, including but not limited to, Escherichia coli. In some embodiments, the bacterial species is Escherichia coli. In some embodiments, the bacterial species is Escherichia coli strain Nissle.

Assays for testing the activity of a cysteine transporter, a functional variant of a cysteine transporter, or a functional fragment of transporter of cysteine are well known to one of ordinary skill in the art.

In one embodiment, when the transporter of a cysteine is expressed in the recombinant bacterial cells described herein, the bacterial cells import 10% more cysteine into the bacterial cell when the transporter is expressed than unmodified bacteria of the same bacterial subtype under the same conditions. In another embodiment, when the cysteine transporter is expressed in the recombinant bacterial cells described herein, the bacterial cells import 20%, 30%, 40%, 50%, 60%, 70%, 80%, 90% or 100% more cysteine into the bacterial cell when the transporter is expressed than unmodified bacteria of the same bacterial subtype under the same conditions. In yet another embodiment, when the cysteine transporter is expressed in the recombinant bacterial cells described herein, the bacterial cells import two-fold more cysteine into the cell when the transporter is expressed than unmodified bacteria of the same bacterial subtype under the same conditions. In yet another embodiment, when the cysteine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more cysteine into the cell when the transporter is expressed than unmodified bacteria of the same bacterial subtype under the same conditions.

### 11. Tyrosine Transporters

In one embodiment, the amino acid transporter is a tyrosine transporter. Tyrosine transporters may be expressed or modified in the recombinant bacteria described herein in order to enhance tyrosine transport into the cell. Specifically, when the tyrosine transporter is expressed in the recombinant bacterial cells described herein, the bacterial cells import more tyrosine into the cell when the transporter is expressed than unmodified bacteria of the same bacterial subtype under the same conditions. Thus, the genetically engineered bacteria comprising a heterologous gene encoding a tyrosine transporter which may be used to import tyrosine into the bacteria.

The uptake of tyrosine into bacterial cells is mediated by proteins well known to those of skill in the art. For example, a tyrosine transporter TyrP has been identified in Lactobacillus brevis (Wolken et al., J. Bacteriol., 188 (6): 2198-2206, 2006) and Escherichia coli.

In one embodiment, the at least one gene encoding a tyrosine transporter is the tyrP gene. In one embodiment, the bacterial cell described herein has been genetically engineered to comprise at least one heterologous tyrP gene. In one embodiment, the at least one gene encoding a tyrosine transporter is the Escherichia coli tyrP gene. In one embodiment, the at least one gene encoding a tyrosine transporter is the Lactobacillus brevi tyrP gene.

In one embodiment, the tyrP gene has at least about 80% identity with the sequence of SEQ ID NO:87. Accordingly, in one embodiment, the tyrP gene has at least about 90% identity with the sequence of SEQ ID NO:87. Accordingly, in one embodiment, the tyrP gene has at least about 95% identity with the sequence of SEQ ID NO:87. Accordingly, in one embodiment, the tyrP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 87. In another embodiment, the tyrP gene comprises the sequence of SEQ ID NO:87. In yet another embodiment the tyrP gene consists of the sequence of SEQ ID NO:87.

In some embodiments, the tyrosine transporter is encoded by a tyrosine transporter gene derived from a bacterial genus or species, including but not limited to, Escherichia coli or Lactobacillus brevis. In some embodiments, the bacterial species is Escherichia coli. In some embodiments, the bacterial species is Escherichia coli strain Nissle.

Assays for testing the activity of a tyrosine transporter, a functional variant of a tyrosine transporter, or a functional fragment of a tyrosine transporter are well known to one of ordinary skill in the art. For example, import of tyrosine may be determined using the methods as described in Wolken et al., J. Bacteriol., 188 (6): 2198-2206, 2006, the entire contents of which are expressly incorporated by reference herein. In one embodiment, when the tyrosine transporter is expressed in the recombinant bacterial cells described herein, the bacterial cells import 10% more tyrosine into the bacterial cell when the transporter is expressed than unmodified bacteria of the same bacterial subtype under the same conditions. In another embodiment, when the tyrosine transporter is expressed in the recombinant bacterial cells described herein, the bacterial cells import 20%, 30%, 40%, 50%, 60%, 70%, 80%, 90% or 100% more tyrosine into the bacterial cell when the transporter is expressed than unmodified bacteria of the same bacterial subtype under the same conditions. In yet another embodiment, when the tyrosine transporter is expressed in the recombinant bacterial cells described herein, the bacterial cells import two-fold more tyrosine into the cell when the transporter is expressed than unmodified bacteria of the same bacterial subtype under the same conditions. In yet another embodiment, when the tyrosine transporter is expressed in the recombinant bacterial cells described herein, the bacterial cells import three-fold, four-fold, five-fold, six-fold, seven-fold, cight-fold, nine-fold, ten-fold, fifteen-fold, twenty-fold, thirty-fold, forty-fold, or fifty-fold, more tyrosine into the cell when the transporter is expressed than unmodified bacteria of the same bacterial subtype under the same conditions.

### 12. Phenylalanine Transporters

In one embodiment, the amino acid transporter is a phenylalanine transporter. Phenylalanine transporters may be expressed or modified in the recombinant bacteria described herein in order to enhance phenylalanine transport into the cell. Specifically, when the phenylalanine transporter is expressed in the recombinant bacterial cells described herein, the bacterial cells import more phenylalanine into the cell when the transporter is expressed than unmodified bacteria of the same bacterial subtype under the same conditions. Thus, the genetically engineered bacteria comprising a heterologous gene encoding a phenylalanine transporter which may be used to import phenylalanine into the bacteria.

The uptake of phenylalanine into bacterial cells is mediated by proteins well known to those of skill in the art. For example, a phenylalanine transporter PheP has been identified (Pi et al. (1991) J. Bacteriol. 173 (12): 3622-9; Pi et al. (1996) J. Bacteriol. 178 (9): 2650-5; Pi et al. (1998) J. Bacteriol. 180 (21): 5515-9; and Horsburgh et al. (2004) Infect. Immun. 72 (5): 3073-3076). Additional phenylalanine transporters have been identified and are known in the art.

In one embodiment, the at least one gene encoding a phenylalanine transporter is the pheP gene. In one embodiment, the bacterial cell described herein has been genetically engineered to comprise at least one heterologous pheP gene. In one embodiment, the at least one gene encoding a phenylalanine transporter is the Escherichia coli pheP gene. In one embodiment, the at least one gene encoding a phenylalanine transporter is the Staphylococcus aureus pheP gene. “Phenylalanine transporter” is used to refer to a membrane transport protein that is capable of transporting phenylalanine into bacterial cells (see, e.g., Pi et al., 1991). In Escherichia coli, the pheP gene encodes a high affinity phenylalanine-specific permease responsible for phenylalanine transport (Pi et al., 1998). In some embodiments, the phenylalanine transporter is encoded by a pheP gene derived from a bacterial species, including but not limited to, Acinetobacter calcoaceticus, Salmonella enterica, and Escherichia coli. Other phenylalanine transporters include Aageneral amino acid permease, encoded by the aroP gene, transports three aromatic amino acids, including phenylalanine, with high affinity, and is thought, together with PheP, responsible for the lion share of phenylalanine import. Additionally, a low level of phenylalanine transport activity has been traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF. In some embodiments, the phenylalanine transporter is encoded by a aroP gene derived from a bacterial species. In some embodiments, the phenylalanine transporter is encoded by LIV-binding protein and LS-binding protein and LivHMGF genes derived from a bacterial species. In some embodiments, the genetically engineered bacteria comprise more than one type of phenylalanine transporter, selected from pheP, aroP, and the LIV-I/LS system.

In one embodiment, the pheP gene has at least about 80% identity with the sequence of SEQ ID NO:98. Accordingly, in one embodiment, the pheP gene has at least about 90% identity with the sequence of SEQ ID NO:98. Accordingly, in one embodiment, the pheP gene has at least about 95% identity with the sequence of SEQ ID NO:98. Accordingly, in one embodiment, the pheP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:98. In another embodiment, the pheP gene comprises the sequence of SEQ ID NO: 98. In yet another embodiment the pheP gene consists of the sequence of SEQ ID NO:98.

In some embodiments, the phenylalanine transporter is encoded by a phenylalanine transporter gene derived from a bacterial genus or species, including but not limited to, Escherichia coli or Staphylococcus aureus. In some embodiments, the bacterial species is Escherichia coli. In some embodiments, the bacterial species is Escherichia coli strain Nissle.

Assays for testing the activity of a phenylalanine transporter, a functional variant of a phenylalanine transporter, or a functional fragment of a phenylalanine transporter are well known to one of ordinary skill in the art. For example, import of phenylalanine may be determined using the methods as described in Pi et al. (1998) J. Bacteriol. 180 (21): 5515-9, the entire contents of which are expressly incorporated by reference herein.

In one embodiment, when the phenylalanine transporter is expressed in the recombinant bacterial cells described herein, the bacterial cells import 10% more phenylalanine into the bacterial cell when the transporter is expressed than unmodified bacteria of the same bacterial subtype under the same conditions. In another embodiment, when the phenylalanine transporter is expressed in the recombinant bacterial cells described herein, the bacterial cells import 20%, 30%, 40%, 50%, 60%, 70%, 80%, 90% or 100% more phenylalanine into the bacterial cell when the transporter is expressed than unmodified bacteria of the same bacterial subtype under the same conditions. In yet another embodiment, when the phenylalanine transporter is expressed in the recombinant bacterial cells described herein, the bacterial cells import two-fold more phenylalanine into the cell when the transporter is expressed than unmodified bacteria of the same bacterial subtype under the same conditions. In yet another embodiment, when the phenylalanine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more phenylalanine into the cell when the transporter is expressed than unmodified bacteria of the same bacterial subtype under the same conditions.

In some embodiment, the recombinant bacterial cell comprising a heterologous gene encoding a phenylalanie transporter may be used to treat a disease, condition, and/or symptom associated with cancer, e.g., a cancer described herein. In some embodiments, the recombinant bacterial cells described herein may be used to reduce, ameliorate, or eliminate one or more symptom(s) associated with a cancer.

In some embodiment, the recombinant bacterial cell comprising a heterologous gene encoding a phenylalanine transporter may be used to treat a disease, condition, and/or symptom associated with hyperphenylalaninemia. In some embodiments, the recombinant bacterial cells described herein may be used to reduce, ameliorate, or eliminate one or more symptom(s) associated with hyperphenylalaninemia. In some embodiments, the disease is selected from the group consisting of: phenylketonuria, classical or typical phenylketonuria, atypical phenylketonuria, permanent mild hyperphenylalaninemia, nonphenylketonuria hyperphenylalaninemia, phenylalanine hydroxylase deficiency, cofactor deficiency, dihydropteridine reductase deficiency, tetrahydropterin synthase deficiency, and Segawa's disease. In some embodiments, hyperphenylalaninemia is secondary to other conditions, e.g., liver diseases. In some embodiments, the invention provides methods for reducing, ameliorating, or eliminating one or more symptom(s) associated with these diseases, including but not limited to neurological deficits, mental retardation, encephalopathy, epilepsy, eczema, reduced growth, microcephaly, tremor, limb spasticity, and/or hypopigmentation. In some embodiments, the subject to be treated is a human patient.

It was discovered that PAL1 and PAL3 expressed on a high-copy plasmid and a low-copy plasmid in genetically engineered E. coli Nissle metabolized and reduced phenylalanine to similar levels, and the rate-limiting step of phenylalanine metabolism was phenylalanine availability. Thus, in some embodiments for the treatment of PKU, it is advantageous to increase phenylalanine transport into the cell, thereby enhancing phenylalanine metabolism. Unexpectedly, even low-copy PAL plasmids are capable of almost completely eliminating Phe from a test sample when expressed in conjunction with pheP. Furthermore, there may be additional advantages to using a low-copy PAL-expressing plasmid in conjunction with pheP in order to enhance the stability of PAL expression while maintaining high phenylalanine metabolism, and to reduce negative selection pressure on the transformed bacterium. In alternate embodiments, the phenylalanine transporter is used in conjunction with the high-copy plasmid.

The genetically engineered bacteria further comprise a gene encoding a phenylalanine transporter. Phenylalanine transporters may be expressed or modified in the genetically engineered bacteria of the invention in order to enhance phenylalanine transport into the cell.

PheP is a membrane transport protein that is capable of transporting phenylalanine into bacterial cells (see, e.g., Pi et al., 1991). In some embodiments, the native pheP gene in the genetically modified bacteria of the invention is not modified. In some embodiments, the genetically engineered bacteria of the invention comprise multiple copies of the native pheP gene. In some embodiments, the genetically engineered bacteria of the invention comprise multiple copies of a non-native pheP gene. In some embodiments, the genetically engineered bacteria of the invention comprise a pheP gene that is controlled by its native promoter, an inducible promoter, a promoter that is stronger than the native promoter, e.g., the GlnRS promoter or the P(Bla) promoter, or a constitutive promoter. In some embodiments, expression of the pheP gene is controlled by a different promoter than the promoter that controls expression of the gene encoding the phenylalanine-metabolizing enzyme and/or the transcriptional regulator. In some embodiments, expression of the pheP gene is controlled by the same promoter that controls expression of the phenylalanine-metabolizing enzyme and/or the transcriptional regulator. In some embodiments, the pheP gene and the phenylalanine-metabolizing enzyme and/or the transcriptional regulator are divergently transcribed from a promoter region. In some embodiments, expression of each of the genes encoding PheP, the phenylalanine-metabolizing enzyme, and the transcriptional regulator is controlled by a different promoter. In some embodiments, expression of the genes encoding PheP, the phenylalanine-metabolizing enzyme, and the transcriptional regulator is controlled by the same promoter.

In some embodiments, the native pheP gene in the genetically modified bacteria is not modified, and one or more additional copies of the native pheP gene are inserted into the genome under the control of the same inducible promoter that controls expression of PAL, e.g., the FNR promoter, or a different inducible promoter than the one that controls expression of PAL, or a constitutive promoter. In alternate embodiments, the native pheP gene is not modified, and a copy of a non-native pheP gene from a different bacterial species is inserted into the genome under the control of the same inducible promoter that controls expression of PAL, e.g., the FNR promoter, or a different inducible promoter than the one that controls expression of PAL, or a constitutive promoter.

In some embodiments, the native pheP gene in the genetically modified bacteria is not modified, and one or more additional copies of the native pheP gene are present in the bacteria on a plasmid and under the control of the same inducible promoter that controls expression of PAL, e.g., the FNR promoter, or a different inducible promoter than the one that controls expression of the PME, or a constitutive promoter. In alternate embodiments, the native pheP gene is not modified, and a copy of a non-native pheP gene from a different bacterial species is present in the bacteria on a plasmid and under the control of the same inducible promoter that controls expression of PAL, e.g., the FNR promoter, or a different inducible promoter than the one that controls expression of PAL, or a constitutive promoter.

In some embodiments, the native pheP gene is mutagenized, mutants exhibiting increased phenylalanine transport are selected, and the mutagenized pheP gene is isolated and inserted into the genetically engineered bacteria (see, e.g., Pi et al., 1996; Pi et al., 1998). The phenylalanine transporter modifications described herein may be present on a plasmid or chromosome.

In some embodiments, the genetically engineered bacterium is E. coli Nissle, and the native pheP gene in E. coli Nissle is not modified; one or more additional copies the native E. coli Nissle pheP genes are inserted into the E. coli Nissle genome under the control of the same inducible promoter that controls expression of PAL, e.g., the FNR promoter, or a different inducible promoter than the one that controls expression of PAL, or a constitutive promoter. In an alternate embodiment, the native pheP gene in E. coli Nissle is not modified, and a copy of a non-native pheP gene from a different bacterium is inserted into the E. coli Nissle genome under the control of the same inducible promoter that controls expression of PAL, e.g., the FNR promoter, or a different inducible promoter than the one that controls expression of PAL, or a constitutive promoter. In some embodiments, the genetically engineered bacterium is E. coli Nissle, and the native pheP gene in E. coli Nissle is not modified; one or more additional copies the native E. coli Nissle pheP genes are present in the bacterium on a plasmid and under the control of the same inducible promoter that controls expression of PAL, e.g., the FNR promoter, or a different inducible promoter than the one that controls expression of PAL, or a constitutive promoter. In an alternate embodiment, the native pheP gene in E. coli Nissle is not modified, and a copy of a non-native pheP gene from a different bacterium, are present in the bacterium on a plasmid and under the control of the same inducible promoter that controls expression of PAL, e.g., the FNR promoter, or a different inducible promoter than the one that controls expression of PAL, or a constitutive promoter.

It has been reported that Escherichia coli has five distinct transport systems (AroP, Mtr, PheP, TnaB, and TyrP) for the accumulation of aromatic amino acids. A general amino acid permease, encoded by the aroP gene, transports three aromatic amino acids, including phenylalanine, with high affinity, and is thought, together with PheP, responsible for the lion share of phenylalanine import. Additionally, a low level of accumulation of phenylalanine was observed in an aromatic amino acid transporter-deficient E. coli strain (ΔaroP ΔpheP Δmtr Δtna ΔtyrP), and was traced to the activity of the LIV-I/LS system, which is a branched-chain amino acid transporter consisting of two periplasmic binding proteins, the LIV-binding protein (LIV-I system) and LS-binding protein (LS system), and membrane components, LivHMGF (Koyanagi et al., and references therein; Identification of the LIV-I/LS System as the Third Phenylalanine Transporter in Escherichia coli K-12).

In some embodiments, the genetically engineered bacteria comprise an aroP gene. In some embodiments, the genetically engineered bacterium is E. coli Nissle, and the native aroP gene in E. coli Nissle is not modified; one or more additional copies the native E. coli Nissle aroP genes are present in the bacterium on a plasmid and under the control of the same inducible promoter that controls expression of the PME, e.g., the FNR promoter, or the araBAD promoter, a different inducible promoter than the one that controls expression of the PME, or a constitutive promoter. In an alternate embodiment, the native aroP gene in E. coli Nissle is not modified, and a copy of a non-native aroP gene from a different bacterium, are present in the bacterium on a plasmid and under the control of the same inducible promoter that controls expression of the PME, e.g., the FNR promoter or the AraBAD promoter, or a different inducible promoter than the one that controls expression of the PME, or a constitutive promoter.

In other embodiments, the genetically engineered bacteria comprise AroP and PheP, under the control of the same or different inducible or constitutive promoters.

In some embodiments, the pheP gene is expressed on a chromosome. In some embodiments, expression from the chromosome may be useful for increasing stability of expression of pheP. In some embodiments, the pheP gene is integrated into the bacterial chromosome at one or more integration sites in the genetically engineered bacteria. In some embodiments, the pheP gene is inserted into the bacterial genome at one or more of the following insertion sites in E. coli Nissle: malE/K, insB/I, araC/BAD, lacZ, agal/rsml, thyA, and malP/T. Any suitable insertion site may be used (see, e.g., The insertion site may be anywhere in the genome, e.g., in a gene required for survival and/or growth, such as thyA (to create an auxotroph); in an active area of the genome, such as near the site of genome replication; and/or in between divergent promoters in order to reduce the risk of unintended transcription, such as between AraB and AraC of the arabinose operon.

In some embodiments, the genetically engineered bacterium comprises multiple mechanisms of action and/or one or more auxotrophies. In certain embodiments, the bacteria are genetically engineered to comprise five copies of PAL under the control of an oxygen level-dependent promoter (e.g., P_{fnrS}-PAL3) inserted at different integration sites on the chromosome (e.g., malE/K, yicS/nepI, malP/T, agaI/rsmI, and cea), and one copy of a phenylalanine transporter gene under the control of an oxygen level-dependent promoter (e.g., P_{fnrS}-pheP) inserted at a different integration site on the chromosome (e.g., lacZ). In a more specific aspect, the bacteria are genetically engineered to further include a kanamycin resistance gene, and a thyA auxotrophy, in which the thyA gene is deleted and/or replaced with an unrelated gene.

### 13. Glutamic Acid Transporters

In one embodiment, the amino acid transporter is a glutamic transporter. Glutamic acid transporters may be expressed or modified in the recombinant bacteria described herein in order to enhance glutamic acid transport into the cell. Specifically, when the glutamic acid transporter is expressed in the recombinant bacterial cells described herein, the bacterial cells import more glutamic acid into the cell when the transporter is expressed than unmodified bacteria of the same bacterial subtype under the same conditions. Thus, the genetically engineered bacteria comprising a heterologous gene encoding a glutamic acid transporter which may be used to import glutamic acid into the bacteria.

The uptake of glutamic acid into bacterial cells is mediated by proteins well known to those of skill in the art. For example, a Na⁺-coupled symporter GltT for glutamic acid uptake has been identified in Bacillus subtilis (see, e.g., Zaprasis et al. (2015) App. Env. Microbiol. 81:250-9). The bacterial gene gltT encodes a glutamic acid transporter responsible for glutamic acid uptake in many bacteria (see, e.g., Jan Slotboom et al. (1999) Microb. Mol. Biol. Rev. 63:293-307; Takahashi et al. (2015) Inf. Imm. 83:3555-67; Ryan et al. (2007) Nat. Struct. Mol. Biol. 14:365-71; and Tolner et al. (1992) Mol. Microbiol. 6:2845-56).

In one embodiment, the at least one gene encoding a glutamic acid transporter is the gltT gene. In one embodiment, the bacterial cell described herein has been genetically engineered to comprise at least one heterologous gltT gene. In one embodiment, the at least one gene encoding a glutamic acid transporter is the Escherichia coli gltP gene. In one embodiment, the at least one gene encoding a glutamic acid transporter is the Bacillus subtilis gltT gene. In one embodiment, the at least one gene encoding a glutamic acid transporter is the Mycobacterium tuberculosis detA gene. In one embodiment, the at least one gene encoding a glutamic acid transporter is the Salmonella typhimurium detA gene. In one embodiment, the at least one gene encoding a glutamic acid transporter is the Caenorhabditis elegans gltT gene.

In one embodiment, the gltT gene has at least about 80% identity with the sequence of SEQ ID NO:91. Accordingly, in one embodiment, the gltT gene has at least about 90% identity with the sequence of SEQ ID NO:91. Accordingly, in one embodiment, the gltT gene has at least about 95% identity with the sequence of SEQ ID NO:91. Accordingly, in one embodiment, the gltT gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 91. In another embodiment, the gltT gene comprises the sequence of SEQ ID NO:91. In yet another embodiment the gltT gene consists of the sequence of SEQ ID NO:91.

In some embodiments, the glutamic acid transporter is encoded by a glutamic acid transporter gene derived from a bacterial genus or species, including but not limited to, Escherichia, Bacillus, Chlamydia, Mycobacterium, Salmonella, Escherichia coli, Mycobacterium tuberculosis, Salmonella typhimurium, or Caenorhabditis elegans (see, e.g., Jan Slotboom et al. (1999) Microbiol. Mol. Biol. Rev. 63:293-307) In some embodiments, the bacterial species is Escherichia coli. In some embodiments, the bacterial species is Escherichia coli strain Nissle.

Assays for testing the activity of a glutamic acid transporter, a functional variant of a glutamic acid transporter, or a functional fragment of transporter of glutamic acid are well known to one of ordinary skill in the art. For example, import of glutamic acid may be determined using the methods as described in Zaprasis et al. (2015) App. Env. Microbiol. 81:250-9, the entire contents of each of which are expressly incorporated by reference herein.

In one embodiment, when the glutamic acid transporter is expressed in the recombinant bacterial cells described herein, the bacterial cells import 10% more glutamic acid into the bacterial cell when the transporter is expressed than unmodified bacteria of the same bacterial subtype under the same conditions. In another embodiment, when the glutamic acid transporter is expressed in the recombinant bacterial cells described herein, the bacterial cells import 20%, 30%, 40%, 50%, 60%, 70%, 80%, 90% or 100% more glutamic acid into the bacterial cell when the transporter is expressed than unmodified bacteria of the same bacterial subtype under the same conditions. In yet another embodiment, when the glutamic acid transporter is expressed in the recombinant bacterial cells described herein, the bacterial cells import two-fold more glutamic acid into the cell when the transporter is expressed than unmodified bacteria of the same bacterial subtype under the same conditions. In yet another embodiment, when the glutamic acid transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more glutamic acid into the cell when the transporter is expressed than unmodified bacteria of the same bacterial subtype under the same conditions.

### 14. Histidine Transporters

In one embodiment, the amino acid transporter is a histidine transporter. Histidine transporters may be expressed or modified in the recombinant bacteria described herein in order to enhance histidine transport into the cell. Specifically, when the histidine transporter is expressed in the recombinant bacterial cells described herein, the bacterial cells import more histidine into the cell when the transporter is expressed than unmodified bacteria of the same bacterial subtype under the same conditions. Thus, the genetically engineered bacteria comprising a heterologous gene encoding a histidine transporter which may be used to import histidine into the bacteria.

The uptake of histidine into bacterial cells is mediated by proteins well known to those of skill in the art. For example, a histidine transport system is encoded by the hisJQMP operon and the art gene (see, e.g., Caldara et al. (2007) J. Mol. Biol. 373 (2): 251-67). Transport by the histidine transport system is mediated by several proteins regulated by the ArgR-L-arginine DNA-binding transcriptional dual regulator. ArgR complexed with L-arginine represses the transcription of several genes involved in transport of histidine. In this system, HisJ (encoded by hisJ) is a histidine ABC transporter-periplasmic binding protein, HisQ and HisM (encoded by hisQ and hisM respectively) are the lysine/arginine/ornithine ABC transporter/histidine ABC transporter-membrane subunits, HisP (encoded by hisP) is a lysine/arginine/ornithine ABC transporter/histidine ABC transporter-ATP binding subunit. This system has been well characterized in Escherichia coli. In addition, bacterial systems that are homologous and orthologous to the E. coli histidine-specific system have been characterized in other bacterial species, including, for example, Pseudomonas fluorescens (see, e.g., Bender (2012) Microbiol. Mol. Biol. Reviews 76:565-584). The membranous and membrane-associated proteins of the histidine permease (Q M P complex), encoded by the hisJQMP operon mediate the uptake of histidine (see, e.g., Oh et al. (1994) J. Biol. Chem. 269 (42): 26323-30).

In one embodiment, the at least one gene encoding a histidine transporter comprises the hisJQMP operon. In one embodiment, the at least one gene encoding a histidine transporter comprises the hisJ gene. In one embodiment, the at least one gene encoding a histidine transporter comprises the hisQ gene. In one embodiment, the at least one gene encoding a histidine transporter comprises the hisM gene. In one embodiment, the at least one gene encoding a histidine transporter comprises the hisP gene.

In one embodiment, the hisJ gene has at least about 80% identity with the sequence of SEQ ID NO:94. Accordingly, in one embodiment, the his) gene has at least about 90% identity with the sequence of SEQ ID NO:94. Accordingly, in one embodiment, the hisJ gene has at least about 95% identity with the sequence of SEQ ID NO:94. Accordingly, in one embodiment, the hisJ gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 94. In another embodiment, the hisJ gene comprises the sequence of SEQ ID NO:94. In yet another embodiment, the his gene consists of the sequence of SEQ ID NO:94.

In one embodiment, the hisQ gene has at least about 80% identity with the sequence of SEQ ID NO:95. Accordingly, in one embodiment, the hisQ gene has at least about 90% identity with the sequence of SEQ ID NO:95. Accordingly, in one embodiment, the hisQ gene has at least about 95% identity with the sequence of SEQ ID NO:95. Accordingly, in one embodiment, the hisQ gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 95. In another embodiment, the hisQ gene comprises the sequence of SEQ ID NO:95. In yet another embodiment, the hisQ gene consists of the sequence of SEQ ID NO:95.

In one embodiment, the hisM gene has at least about 80% identity with the sequence of SEQ ID NO:103. Accordingly, in one embodiment, the hisM gene has at least about 90% identity with the sequence of SEQ ID NO: 103. Accordingly, in one embodiment, the hisM gene has at least about 95% identity with the sequence of SEQ ID NO: 103. Accordingly, in one embodiment, the hisM gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 103. In another embodiment, the hisM gene comprises the sequence of SEQ ID NO: 103. In yet another embodiment, the hisM gene consists of the sequence of SEQ ID NO: 103.

In one embodiment, the hisP gene has at least about 80% identity with the sequence of SEQ ID NO:96. Accordingly, in one embodiment, the hisP gene has at least about 90% identity with the sequence of SEQ ID NO:96. Accordingly, in one embodiment, the hisP gene has at least about 95% identity with the sequence of SEQ ID NO:96. Accordingly, in one embodiment, the hisP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 96. In another embodiment, the hisP gene comprises the sequence of SEQ ID NO:96. In yet another embodiment, the hisP gene consists of the sequence of SEQ ID NO:96.

In some embodiments, the histidine transporter is encoded by a histidine transporter gene derived from a bacterial genus or species, including but not limited to, Escherichia and Pseudomonas In some embodiments, the bacterial species is Escherichia coli. In some embodiments, the bacterial species is Escherichia coli strain Nissle.

Assays for testing the activity of a histidine transporter, a functional variant of a histidine transporter, or a functional fragment of a histidine transporter are well known to one of ordinary skill in the art. For example, import of histidine may be determined using the methods described in Liu et al. (1997) J. Biol. Chem. 272:859-866 and Shang et al. (2013) J. Bacteriology. 195 (23): 5334-5342, the entire contents of each of which are expressly incorporated by reference herein.

In one embodiment, when the histidine transporter is expressed in the recombinant bacterial cells described herein, the bacterial cells import 10% more histidine into the bacterial cell when the transporter is expressed than unmodified bacteria of the same bacterial subtype under the same conditions. In another embodiment, when the histidine transporter is expressed in the recombinant bacterial cells described herein, the bacterial cells import 20%, 30%, 40%, 50%, 60%, 70%, 80%, 90% or 100% more histidine into the bacterial cell when the transporter is expressed than unmodified bacteria of the same bacterial subtype under the same conditions. In yet another embodiment, when the histidine transporter is expressed in the recombinant bacterial cells described herein, the bacterial cells import two-fold more histidine into the cell when the transporter is expressed than unmodified bacteria of the same bacterial subtype under the same conditions. In yet another embodiment, when the histidine transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more histidine into the cell when the transporter is expressed than unmodified bacteria of the same bacterial subtype under the same conditions.

### 15. Proline Transporters

In one embodiment, the amino acid transporter is a proline transporter. Proline transporters may be expressed or modified in the recombinant bacteria described herein in order to enhance proline transport into the cell. Specifically, when the proline transporter is expressed in the recombinant bacterial cells described herein, the bacterial cells import more proline into the cell when the transporter is expressed than unmodified bacteria of the same bacterial subtype under the same conditions. Thus, the genetically engineered bacteria comprising a heterologous gene encoding a proline transporter which may be used to import proline into the bacteria.

The uptake of proline into bacterial cells is mediated by proteins well known to those of skill in the art. The proline utilization operon (put) allows bacterial cells to transport and use proline. The put operon consists of two genes putA and putP. In bacteria, there are two distinct systems for proline uptake, proline porter I (PPI) and proline porter II (PPII) (see, e.g., Grothe (1986) J. Bacteriol. 166:253-259). The bacterial gene putP encodes a proline transporter responsible for proline uptake in many bacteria (see, e.g., Ostrovsky et al. (1993) Proc. Natl. Acad. Sci. 90:429-8; Grothe (1986) J. Bacteriol. 166:253-259). The putA gene expresses a polypeptide that has proline dehydrogenase (EC 1.5.99.8) activity and pyrroline-5-carboxylate (P5C) (EC 1.5.1.12) activity (see, e.g., Menzel and Roth (1981) J. Biol. Chem. 256:9755-61). In the absence of proline, putA remains in the cytoplasm and represses put gene expression. In the presence of proline, putA binds to the membrane relieving put repression allowing put gene expression (see, e.g., Ostrovsky et al. (1993) Proc. Natl. Acad. Sci. 90:429-8).

In one embodiment, the at least one gene encoding a proline transporter is the putP gene. In one embodiment, the bacterial cell described herein has been genetically engineered to comprise at least one heterologous putP gene. In one embodiment, the at least one gene encoding a proline transporter is the Escherichia coli putP gene. In one embodiment, the at least one gene encoding a proline transporter is the Salmonella typhimurium putP gene. In one embodiment, the at least one gene encoding a proline transporter is the Escherichia coli putP gene.

In one embodiment, the putP gene has at least about 80% identity with the sequence of SEQ ID NO:98. Accordingly, in one embodiment, the putP gene has at least about 90% identity with the sequence of SEQ ID NO:98. Accordingly, in one embodiment, the putP gene has at least about 95% identity with the sequence of SEQ ID NO:98. Accordingly, in one embodiment, the putP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 98. In another embodiment, the putP gene comprises the sequence of SEQ ID NO:98. In yet another embodiment the putP gene consists of the sequence of SEQ ID NO:98.

In some embodiments, the proline transporter is encoded by a proline transporter gene derived from a bacterial genus or species, including but not limited to, Escherichia, Salmonella, Escherichia coli or Salmonella typhimurium. In some embodiments, the bacterial species is Escherichia coli. In some embodiments, the bacterial species is Escherichia coli strain Nissle.

Assays for testing the activity of a proline transporter, a functional variant of a proline transporter, or a functional fragment of a proline transporter are well known to one of ordinary skill in the art. For example, import of proline may be determined using the methods as described in Moses et al. (2012) Journal of Bacteriology 194:745-58 and Hoffman et al. (2012) App. and Enviro. Microbiol. 78:5753-62), the entire contents of each of which are expressly incorporated by reference herein.

In one embodiment, when the proline transporter is expressed in the recombinant bacterial cells described herein, the bacterial cells import 10% more proline into the bacterial cell when the transporter is expressed than unmodified bacteria of the same bacterial subtype under the same conditions. In another embodiment, when the proline transporter is expressed in the recombinant bacterial cells described herein, the bacterial cells import 20%, 30%, 40%, 50%, 60%, 70%, 80%, 90% or 100% more proline into the bacterial cell when the transporter is expressed than unmodified bacteria of the same bacterial subtype under the same conditions. In yet another embodiment, when the proline transporter is expressed in the recombinant bacterial cells described herein, the bacterial cells import two-fold more proline into the cell when the transporter is expressed than unmodified bacteria of the same bacterial subtype under the same conditions. In yet another embodiment, when the proline transporter is expressed in the recombinant bacterial cells described herein, the bacterial cells import three-fold, four-fold, five-fold, six-fold, seven-fold, eight-fold, nine-fold, ten-fold, fifteen-fold, twenty-fold, thirty-fold, forty-fold, or fifty-fold, more proline into the cell when the transporter is expressed than unmodified bacteria of the same bacterial subtype under the same conditions.

### B. Nucleoside Transporters

In one embodiment, the recombinant bacterial cell of the invention comprises a heterologous gene encoding a substrate transporter, wherein the substrate transporter is a nucleoside transporter. In one embodiment, the nucleoside transporter is a purine nucleoside transporter. In one embodiment, the nucleoside transporter is a pyrimidine nucleoside transporter. In one embodiment, the nucleoside transporter transports at least one nucleoside selected from the group consisting of adenosine, guanosine, uridine, inosine, xanthosine, thymidine and cytidine, into the cell.

The uptake of nucleosides into bacterial cells is mediated by proteins well known to those of skill in the art. For example, many bacteria scavenge nucleosides from the environment for the synthesis of nucleotides and deoxynucleotides. In some bacterial species, e.g., Escherichia coli, nucleosides can be used as the sole source of nitrogen and carbon for growth (see, e.g., Neuhard and Nygaard “Biosynthesis and conversion of nucleotides, purines and pyrimidines,” in: Neidhardt FC, Ingraham JL, Low KB, Magasanik B, Schaechter M, Umbarger HE, editors. Escherichia coli and Salmonella typhimurium: Cellular and Molecular Biology. Washington DC: ASM Press; 1987. pp. 445-473). Two evolutionarily unrelated cation-linked transporter families have been identified: the concentrative nucleoside transporter (CNT) family and the nucleoside:H⁺ Symporter (NHS) family, both of which are responsible for nucleoside uptake (see, e.g., Cabrita et al. (2002) Biochem. Cell Biol. 80 (5): 623-38, the contents of which is herein incorporated by reference in its entirety).

Passive transport of nucleosides across the outer membrane of some Gram-negative bacteria, e.g., Salmonella enterica, and into the periplasm can be mediated by the Tsx porin, encoded by the tsx gene (see, e.g., Bucarey et al. (20005) Infect. Immun. 73 (10): 6210-9).

Active transport of nucleosides across the inner membrance is mediated by the nucleoside permeases NupC and NupG, encoded by the nupC and nupG genes, respectively. NupG can facilitate the uptake of all tested purine and pyrimidine nucleosides while NupC has specificity towards the pyrimidine nucleosides and their deoxyderivatives. Both permeases are powered by proton motive force. E. coli mutants defective in both the nupC and nupG genes cannot grow with nucleosides as their single carbon source. Both permeases are proton-linked but they differ in their selectivity. NupG is capable of transporting a wide range of nucleosides and deoxynucleosides; in contrast, NupC does not transport guanosine or deoxyguanosine. Homologs of NupG from E. coli are found in a wide range of bacteria, including human gut pathogens such as Salmonella typhimurium, organisms associated with periodontal disease such as Porphyromonas gingivalis and Prevotella intermedia, and plant pathogens of the genus Erwinia (see, e.g., Vaziri et al. (2013) Mol. Membr. Biol. 30 (1-2): 114-128, the contents of which is herein incorporated by reference in its entirety).

An additional nucleoside transporter, the xanthosine permease, XapB, having 58% identity to NupG was identified in Escherichia coli Norholm and Dandanell (2001) J. Bacteriol. 183 (16): 4900-4. XapB exhibits similar specificity to NupG, since it appears to be able to transport all nucleosides except guanosine. Putative bacterial transporters from the CNT superfamily and transporters from the NupG/XapB family include those listed in the tables 2 and 3 below. In addition, codB (GenBank P25525, Escherichia coli) was identified based on homology to a yeast transporter family termed the uracil/allantoin transportor family (Cabrita et al. (2002)).

TABLE 2
Putative CNT family transporters

| Name | GenBank Accession No. | Organism |
|---|---|---|
| BH1446 | BAB05165 | Bacillus halodurans |
| BsNupC | CAA57663 | Bacillus subtilis |
| BsyutK | CAB15208 | B. subtilis |
| BsyxjA | CAB15938 | B. subtilis |
| CcCNT (CC2089) | AAK24060 | Caulobacter crescentus |
| yeiJ | AAC75222 | E. coli |
| yeiM | AAC75225 | E. coli |
| HI0519 | AAC22177 | Haemophilus influenzae |
| HP1180 (NupC) | AAD08224 | Helicobacter pylori |
| SA0600 (NupC) | BAB41833 | Staphylococcus aureus |
| SAV0645 (NupC) | BAB56807 | S. aureus |
| SpNupC | AAK34582 | Streptococcus pyogenes |
| VC2352 (NupC) | AAF95495 | Vibrio cholerae |
| VC1953 (NupC) | AAF95101 | V. cholera |
| VCA0179 | AAF96092 | V. cholera |

TABLE 3
Bacterial transporters from the NupG/XapB family

| Protein | GenBank | |
|---|---|---|
| (gene name) | Accession No. | Organism |
| yegT | P76417 | Escherichia coli |
| NupG | P09452 | E. coli |
| XapB | P45562 | E. coli |
| CC1628 | AAK23606 | Caulobacter crescentus |

In one embodiment, the nucleoside transporter is a nucleoside permease (e.g., NupC or NupG). In one embodiment, the nucleoside transporter is a adenosine permease. In one embodiment, the nucleoside transporter is a guanosine permease. In one embodiment, the nucleoside transporter is a uridine permease. In one embodiment, the nucleoside transporter is a inosine permease. In one embodiment, the nucleoside transporter is a xanthosine permease. In one embodiment, the nucleoside transporter is a thymidine permease. In one embodiment, the nucleoside transporter is a cytidine permease.

In one embodiment, the nucleoside transporter is a nucleoside porin (e.g., Tsx). In one embodiment, the nucleoside transporter is a sodium-dependent nucleoside transporter. In one embodiment, the nucleoside transporter is a xanthosine transporter (e.g., XapB).

Nucleoside transporters may be expressed or modified in the bacteria in order to enhance nucleoside transport into the cell. Specifically, when the nucleoside transporter is expressed in the recombinant bacterial cells, the bacterial cells import more nucleoside(s) into the cell when the transporter is expressed than unmodified bacteria of the same bacterial subtype under the same conditions. In one embodiment, the bacterial cell comprises a heterologous gene encoding a nucleoside transporter. In one embodiment, the bacterial cell comprises a heterologous gene encoding a nucleoside transporter and a genetic modification that reduces export of a nucleoside, e.g., a genetic mutation in an exporter gene or promoter.

In one embodiment, the bacterial cell comprises at least one gene encoding a nucleoside transporter from a different organism, e.g., a different species of bacteria. In one embodiment, the bacterial cell comprises at least one native gene encoding a nucleoside transporter. In some embodiments, the at least one native gene encoding a nucleoside transporter is not modified. In another embodiment, the bacterial cell comprises more than one copy of at least one native gene encoding a nucleoside transporter. In yet another embodiment, the bacterial cell comprises a copy of at least one gene encoding a native nucleoside transporter, as well as at least one copy of at least one heterologous gene encoding annucleoside transporter from a different bacterial species. In one embodiment, the bacterial cell comprises at least one, two, three, four, five, or six copies of the at least one heterologous gene encoding a nucleoside transporter. In one embodiment, the bacterial cell comprises multiple copies of the at least one heterologous gene encoding a nucleoside transporter.

In one embodiment, the recombinant bacterial cell comprises a heterologous gene encoding a nucleoside transporter, wherein said nucleoside transporter comprises a nucleoside sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the nucleoside sequence of a polypeptide encoded by a nucleoside transporter gene disclosed herein.

In some embodiments, the nucleoside transporter is encoded by a nucleoside transporter gene derived from a bacterial genus or species, including but not limited to, Bacillus halodurans, Bacillus subtilis, Caulobacter crescentus, Escherichia coli, Haemoophilus influenzae, Helicobacter pylori, Pseudomonas, Bacillus subtilis, Escherichia coli, Prevotella intermedia, Porphytomonas gingivalis, Salmonella typhimurium, Salmonella enterica, or Vibrio cholera. In some embodiments, the bacterial species is Escherichia coli. In some embodiments, the bacterial species is Escherichia coli strain Nissle.

The present disclosure further comprises genes encoding functional fragments of a nucleoside transporter or functional variants of a nucleoside transporter. As used herein, the term “functional fragment thereof” or “functional variant thereof” of a nucleoside transporter relates to an element having qualitative biological activity in common with the wild-type nucleoside transporter from which the fragment or variant was derived. For example, a functional fragment or a functional variant of a mutated nucleoside transporter is one which retains essentially the same ability to import a nucleoside into the bacterial cell as does the nucleoside transporter protein from which the functional fragment or functional variant was derived. In one embodiment, the recombinant bacterial cell comprises at least one heterologous gene encoding a functional fragment of a nucleoside transporter. In another embodiment, the recombinant bacterial cell comprises a heterologous gene encoding a functional variant of a nucleoside transporter.

Assays for testing the activity of a nucleoside transporter, a functional variant of a nucleoside transporter, or a functional fragment of a nucleoside transporter are well known to one of ordinary skill in the art. For example, import of a nucleoside may be determined using, e.g., a ¹⁴C-labeled nucleoside uptake assay as described in Norholm and Dandanell (2001) J. Bacteriol. 183 (16): 4900-4, the entire contents of each of which are expressly incorporated by reference herein.

In one embodiment, the genes encoding the nucleoside transporter have been codon-optimized for use in the host organism, e.g., a bacterial cell disclosed herein. In one embodiment, the genes encoding the nucleoside transporter have been codon-optimized for use in Escherichia coli.

The present disclosure also encompasses genes encoding a nucleoside transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a nucleoside transporter is mutagenized; mutants exhibiting increased nucleoside import are selected; and the mutagenized at least one gene encoding a nucleoside transporter is isolated and inserted into the bacterial cell. In some embodiments, the at least one gene encoding a nucleoside transporter is mutagenized; mutants exhibiting decreased nucleoside import are selected; and the mutagenized at least one gene encoding a nucleoside transporter is isolated and inserted into the bacterial cell. The transporter modifications described herein may be present on a plasmid or chromosome.

In some embodiments, the bacterial cell comprises a heterologous gene encoding a nucleoside transporter operably linked to a promoter. In one embodiment, the at least one gene encoding a nucleoside transporter is directly operably linked to the promoter. In another embodiment, the at least one gene encoding a nucleoside transporter is indirectly operably linked to the promoter.

In one embodiment, the promoter is not operably linked with the at least one gene encoding a nucleoside transporter in nature. In some embodiments, the at least one gene encoding the nucleoside transporter is controlled by its native promoter. In some embodiments, the at least one gene encoding the nucleoside transporter is controlled by an inducible promoter. In some embodiments, the at least one gene encoding the nucleoside transporter is controlled by a promoter that is stronger than its native promoter. In some embodiments, the at least one gene encoding the nucleoside transporter is controlled by a constitutive promoter.

In another embodiment, the promoter is an inducible promoter. Inducible promoters are described in more detail infra.

In one embodiment, the at least one gene encoding a nucleoside transporter is located on a plasmid in the bacterial cell. In some embodiments, the plasmid is a high copy number plasmid. In some embodiments, the plasmid is a low copy number plasmid. In another embodiment, the at least one gene encoding a nucleoside transporter is located in the chromosome of the bacterial cell. In yet another embodiment, a native copy of the at least one gene encoding a nucleoside transporter is located in the chromosome of the bacterial cell, and a copy of at least one gene encoding a nucleoside transporter from a different species of bacteria is located on a plasmid in the bacterial cell. In yet another embodiment, a native copy of the at least one gene encoding a nucleoside transporter is located on a plasmid in the bacterial cell, and a copy of at least one gene encoding a nucleoside transporter from a different species of bacteria is located on a plasmid in the bacterial cell. In yet another embodiment, a native copy of the at least one gene encoding a nucleoside transporter is located in the chromosome of the bacterial cell, and a copy of the at least one gene encoding a nucleoside transporter from a different species of bacteria is located in the chromosome of the bacterial cell.

In some embodiments, the at least one native gene encoding the nucleoside transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the nucleoside transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the nucleoside transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the nucleoside transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In one embodiment, the nucleoside transporter is encoded by a tsx gene, e.g., a tsx gene disclosed herein. In one embodiment, the tsx gene has at least about 80% identity with the sequence of SEQ ID NO: 107. Accordingly, in one embodiment, the tsx gene has at least about 90% identity with the sequence of SEQ ID NO: 107. Accordingly, in one embodiment, the tsx gene has at least about 95% identity with the sequence of SEQ ID NO: 107. Accordingly, in one embodiment, the tsx gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 107. In another embodiment, the tsx gene comprises the sequence of SEQ ID NO:107. In yet another embodiment the tsx gene consists of the sequence of SEQ ID NO: 107.

In one embodiment, the tsx gene has at least about 80% identity with the sequence of SEQ ID NO: 108. Accordingly, in one embodiment, the tsx gene has at least about 90% identity with the sequence of SEQ ID NO: 108. Accordingly, in one embodiment, the tsx gene has at least about 95% identity with the sequence of SEQ ID NO: 108. Accordingly, in one embodiment, the tsx gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 108. In another embodiment, the tsx gene comprises the sequence of SEQ ID NO:108. In yet another embodiment the tsx gene consists of the sequence of SEQ ID NO:108.

In one embodiment, the nucleoside transporter is encoded by a BH1446 gene, e.g., a BH1446 gene disclosed herein. In one embodiment, the BH1446 gene has at least about 80% identity with the sequence of SEQ ID NO: 109. Accordingly, in one embodiment, the BH1446 gene has at least about 90% identity with the sequence of SEQ ID NO:109. Accordingly, in one embodiment, the BH1446 gene has at least about 95% identity with the sequence of SEQ ID NO:109. Accordingly, in one embodiment, the BH1446 gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:109. In another embodiment, the BH1446 gene comprises the sequence of SEQ ID NO: 109. In yet another embodiment the BH1446 gene consists of the sequence of SEQ ID NO:109.

In one embodiment, the nucleoside transporter is encoded by a nupC gene, e.g., a nupC gene disclosed herein. In one embodiment, the nupC gene is a nupC gene from Bacillus subtilis. In one embodiment, the nupC gene has at least about 80% identity with the sequence of SEQ ID NO:110. Accordingly, in one embodiment, the nupC gene has at least about 90% identity with the sequence of SEQ ID NO:110. Accordingly, in one embodiment, the nupC gene has at least about 95% identity with the sequence of SEQ ID NO:110. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:110. In another embodiment, the nupC gene comprises the sequence of SEQ ID NO:110. In yet another embodiment the nupC gene consists of the sequence of SEQ ID NO:110.

In one embodiment, the nupC gene is a nupC gene from Helicobacter pylori. In one embodiment, the nupC gene has at least about 80% identity with the sequence of SEQ ID NO: 117. Accordingly, in one embodiment, the nupC gene has at least about 90% identity with the sequence of SEQ ID NO: 117. Accordingly, in one embodiment, the nupC gene has at least about 95% identity with the sequence of SEQ ID NO:117. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:117. In another embodiment, the nupC gene comprises the sequence of SEQ ID NO:117. In yet another embodiment the nupC gene consists of the sequence of SEQ ID NO:117.

In one embodiment, the nupC (also referred to herein as SA0600) gene is a nupC gene from Staphylococcus aureus. In one embodiment, the nupC gene has at least about 80% identity with the sequence of SEQ ID NO:118. Accordingly, in one embodiment, the nupC gene has at least about 90% identity with the sequence of SEQ ID NO:118. Accordingly, in one embodiment, the nupC gene has at least about 95% identity with the sequence of SEQ ID NO: 118. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 118. In another embodiment, the nupC gene comprises the sequence of SEQ ID NO:118. In yet another embodiment the nupC gene consists of the sequence of SEQ ID NO:118.

In one embodiment, the nupC (also referred to herein as SAV0645) gene is a nupC gene from Staphylococcus aureus. In one embodiment, the nupC gene has at least about 80% identity with the sequence of SEQ ID NO:119. Accordingly, in one embodiment, the nupC gene has at least about 90% identity with the sequence of SEQ ID NO: 119. Accordingly, in one embodiment, the nupC gene has at least about 95% identity with the sequence of SEQ ID NO: 119. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 119. In another embodiment, the nupC gene comprises the sequence of SEQ ID NO:119. In yet another embodiment the nupC gene consists of the sequence of SEQ ID NO:119.

In one embodiment, the nupC (also referred to herein as spNupC) gene is a nupC gene from Streptococcus pyogenes. In one embodiment, the nupC gene has at least about 80% identity with the sequence of SEQ ID NO: 120. Accordingly, in one embodiment, the nupC gene has at least about 90% identity with the sequence of SEQ ID NO:120. Accordingly, in one embodiment, the nupC gene has at least about 95% identity with the sequence of SEQ ID NO: 120. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 120. In another embodiment, the nupC gene comprises the sequence of SEQ ID NO: 120. In yet another embodiment the nupC gene consists of the sequence of SEQ ID NO:120.

In one embodiment, the nupC (also referred to herein as VC2352) gene is a nupC gene from Vibrio cholerae. In one embodiment, the nupC gene has at least about 80% identity with the sequence of SEQ ID NO: 121. Accordingly, in one embodiment, the nupC gene has at least about 90% identity with the sequence of SEQ ID NO:121. Accordingly, in one embodiment, the nupC gene has at least about 95% identity with the sequence of SEQ ID NO: 121. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 121. In another embodiment, the nupC gene comprises the sequence of SEQ ID NO:121. In yet another embodiment the nupC gene consists of the sequence of SEQ ID NO: 121.

In one embodiment, the nupC (also referred to herein as VC1953) gene is a nupC gene from Vibrio cholerae. In one embodiment, the nupC gene has at least about 80% identity with the sequence of SEQ ID NO: 122. Accordingly, in one embodiment, the nupC gene has at least about 90% identity with the sequence of SEQ ID NO: 122. Accordingly, in one embodiment, the nupC gene has at least about 95% identity with the sequence of SEQ ID NO: 122. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 122. In another embodiment, the nupC gene comprises the sequence of SEQ ID NO: 122. In yet another embodiment the nupC gene consists of the sequence of SEQ ID NO: 122.

In one embodiment, the nupC (also referred to herein as VCA0179) gene is a nupC gene from Vibrio cholerae. In one embodiment, the nupC gene has at least about 80% identity with the sequence of SEQ ID NO: 123. Accordingly, in one embodiment, the nupC gene has at least about 90% identity with the sequence of SEQ ID NO:123. Accordingly, in one embodiment, the nupC gene has at least about 95% identity with the sequence of SEQ ID NO: 123. Accordingly, in one embodiment, the nupC gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:123. In another embodiment, the nupC gene comprises the sequence of SEQ ID NO: 123. In yet another embodiment the nupC gene consists of the sequence of SEQ ID NO:123.

In one embodiment, the nucleoside transporter is encoded by a yutK gene, e.g., a yutK gene disclosed herein. In one embodiment, the yutK gene is a yutK gene from Bacillus subtilis. In one embodiment, the yutK gene has at least about 80% identity with the sequence of SEQ ID NO:111. Accordingly, in one embodiment, the yutK gene has at least about 90% identity with the sequence of SEQ ID NO:111. Accordingly, in one embodiment, the yutK gene has at least about 95% identity with the sequence of SEQ ID NO:111. Accordingly, in one embodiment, the yutK gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:111. In another embodiment, the yutK gene comprises the sequence of SEQ ID NO: 111. In yet another embodiment the yutK gene consists of the sequence of SEQ ID NO: 111.

In one embodiment, the nucleoside transporter is encoded by a yxjA gene, e.g., a yxjA gene disclosed herein. In one embodiment, the yxjA gene is a yxjA gene from Bacillus subtilis. In one embodiment, the yxjA gene has at least about 80% identity with the sequence of SEQ ID NO:112. Accordingly, in one embodiment, the yxjA gene has at least about 90% identity with the sequence of SEQ ID NO: 112. Accordingly, in one embodiment, the yxjA gene has at least about 95% identity with the sequence of SEQ ID NO: 112. Accordingly, in one embodiment, the yxjA gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 112. In another embodiment, the yxjA gene comprises the sequence of SEQ ID NO: 112. In yet another embodiment the yxjA gene consists of the sequence of SEQ ID NO: 112.

In one embodiment, the nucleoside transporter is encoded by a sodium-dependent nucleoside transporter gene, e.g., a sodium-dependent nucleoside transporter gene disclosed herein. In one embodiment, the sodium-dependent nucleoside transporter gene is a CC2089 (also referred to herein as CcCNT) gene. In one embodiment, the sodium-dependent nucleoside transporter gene is a CC2089 gene from Caulobacter crescentus. In one embodiment, the sodium-dependent nucleoside transporter gene has at least about 80% identity with the sequence of SEQ ID NO:113. Accordingly, in one embodiment, the sodium-dependent nucleoside transporter gene has at least about 90% identity with the sequence of SEQ ID NO:113. Accordingly, in one embodiment, the sodium-dependent nucleoside transporter gene has at least about 95% identity with the sequence of SEQ ID NO:113. Accordingly, in one embodiment, the sodium-dependent nucleoside transporter gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:113. In another embodiment, the sodium-dependent nucleoside transporter gene comprises the sequence of SEQ ID NO:113. In yet another embodiment the sodium-dependent nucleoside transporter gene consists of the sequence of SEQ ID NO: 113.

In one embodiment, the nucleoside transporter is encoded by a yeiJ gene, e.g., a yeiJ gene disclosed herein. In one embodiment, the yeiJ gene is a yeiJ gene from Escherichia coli. In one embodiment, the yeiJ gene has at least about 80% identity with the sequence of SEQ ID NO:114. Accordingly, in one embodiment, the yeiJ gene has at least about 90% identity with the sequence of SEQ ID NO: 114. Accordingly, in one embodiment, the yeiJ gene has at least about 95% identity with the sequence of SEQ ID NO:114. Accordingly, in one embodiment, the yeiJ gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 114. In another embodiment, the yeiJ gene comprises the sequence of SEQ ID NO: 114. In yet another embodiment the yeiJ gene consists of the sequence of SEQ ID NO:114.

In one embodiment, the nucleoside transporter is encoded by a yeiM gene, e.g., a yeiM gene disclosed herein. In one embodiment, the yeiM gene is a yeiM gene from Escherichia coli. In one embodiment, the yeiM gene has at least about 80% identity with the sequence of SEQ ID NO:115. Accordingly, in one embodiment, the yeiM gene has at least about 90% identity with the sequence of SEQ ID NO: 115. Accordingly, in one embodiment, the yeiM gene has at least about 95% identity with the sequence of SEQ ID NO:115. Accordingly, in one embodiment, the yeiM gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:115. In another embodiment, the yeiM gene comprises the sequence of SEQ ID NO:115. In yet another embodiment the yeiM gene consists of the sequence of SEQ ID NO: 115.

In one embodiment, the nucleoside transporter is encoded by a HI0519 gene, e.g., a HI0519 gene disclosed herein. In one embodiment, the HI0519 gene is a HI0519 gene from Haemophilus influenzae. In one embodiment, the HI0519 gene has at least about 80% identity with the sequence of SEQ ID NO: 116. Accordingly, in one embodiment, the HI0519 gene has at least about 90% identity with the sequence of SEQ ID NO:116. Accordingly, in one embodiment, the HI0519 gene has at least about 95% identity with the sequence of SEQ ID NO: 116. Accordingly, in one embodiment, the HI0519 gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:116. In another embodiment, the HI0519 gene comprises the sequence of SEQ ID NO:116. In yet another embodiment the HI0519 gene consists of the sequence of SEQ ID NO:116.

In one embodiment, the nucleoside transporter is encoded by a yegT gene, e.g., a yegT gene disclosed herein. In one embodiment, the yegT gene is a yegT gene from Escherichia coli. In one embodiment, the yegT gene has at least about 80% identity with the sequence of SEQ ID NO: 124. Accordingly, in one embodiment, the yegT gene has at least about 90% identity with the sequence of SEQ ID NO: 124. Accordingly, in one embodiment, the yegT gene has at least about 95% identity with the sequence of SEQ ID NO: 124. Accordingly, in one embodiment, the yegT gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:124. In another embodiment, the yegT gene comprises the sequence of SEQ ID NO:124. In yet another embodiment the yegT gene consists of the sequence of SEQ ID NO: 124.

In one embodiment, the nucleoside transporter is encoded by a nupG gene, e.g., a nupG gene disclosed herein. In one embodiment, the nupG gene is a nupG gene from Escherichia coli. In one embodiment, the nupG gene has at least about 80% identity with the sequence of SEQ ID NO:125. Accordingly, in one embodiment, the nupG gene has at least about 90% identity with the sequence of SEQ ID NO:125. Accordingly, in one embodiment, the nupG gene has at least about 95% identity with the sequence of SEQ ID NO:125. Accordingly, in one embodiment, the nupG gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:125. In another embodiment, the nupG gene comprises the sequence of SEQ ID NO:125. In yet another embodiment the nupG gene consists of the sequence of SEQ ID NO:125.

In one embodiment, the nucleoside transporter is encoded by a xapB gene, e.g., a xapB gene disclosed herein. In one embodiment, the xapB gene is a xapB gene from Escherichia coli. In one embodiment, the xapB gene has at least about 80% identity with the sequence of SEQ ID NO: 126. Accordingly, in one embodiment, the xapB gene has at least about 90% identity with the sequence of SEQ ID NO:126. Accordingly, in one embodiment, the xapB gene has at least about 95% identity with the sequence of SEQ ID NO:126. Accordingly, in one embodiment, the xapB gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 126. In another embodiment, the xapB gene comprises the sequence of SEQ ID NO: 126. In yet another embodiment the xapB gene consists of the sequence of SEQ ID NO: 126.

In one embodiment, the nucleoside transporter is encoded by a CC1628 gene, e.g., a CC1628 gene disclosed herein. In one embodiment, the CC1628 gene is a CC1628 gene from Caulobacter crescentus. In one embodiment, the CC1628 gene has at least about 80% identity with the sequence of SEQ ID NO: 127. Accordingly, in one embodiment, the CC1628 gene has at least about 90% identity with the sequence of SEQ ID NO:127. Accordingly, in one embodiment, the CC1628 gene has at least about 95% identity with the sequence of SEQ ID NO: 127. Accordingly, in one embodiment, the CC1628 gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 127. In another embodiment, the CC1628 gene comprises the sequence of SEQ ID NO: 127. In yet another embodiment the CC1628 gene consists of the sequence of SEQ ID NO: 127.

In one embodiment, the nucleoside transporter is a cytosine permease, e.g., CodB. In one embodiment, the nucleoside transporter is encoded by a codB gene, e.g., a codB gene disclosed herein. In one embodiment, the codB gene is a codB gene from Escherichia coli. In one embodiment, the codB gene has at least about 80% identity with the sequence of SEQ ID NO:128. Accordingly, in one embodiment, the codB gene has at least about 90% identity with the sequence of SEQ ID NO: 128. Accordingly, in one embodiment, the codB gene has at least about 95% identity with the sequence of SEQ ID NO:128. Accordingly, in one embodiment, the codB gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:128. In another embodiment, the codB gene comprises the sequence of SEQ ID NO: 128. In yet another embodiment the codB gene consists of the sequence of SEQ ID NO: 128.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the nucleoside transporter is expressed in the recombinant bacterial cells, the bacterial cells import 10% more nucleosides into the bacterial cell when the transporter is expressed than unmodified bacteria of the same bacterial subtype under the same conditions. In another embodiment, when the nucleoside transporter is expressed in the recombinant bacterial cells, the bacterial cells import 20%, 30%, 40%, 50%, 60%, 70%, 80%, 90% or 100% more nucleosides, into the bacterial cell when the transporter is expressed than unmodified bacteria of the same bacterial subtype under the same conditions. In yet another embodiment, when the nucleoside transporter is expressed in the recombinant bacterial cells, the bacterial cells import two-fold more nucleosides into the cell when the transporter is expressed than unmodified bacteria of the same bacterial subtype under the same conditions. In yet another embodiment, when the nucleoside transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more nucleoside into the cell when the transporter is expressed than unmodified bacteria of the same bacterial subtype under the same conditions.

In one embodiment, the recombinant bacterial cells described herein comprise a first heterologous nucleoside transporter and a second heterologous nucleoside transporter. For, example, in one embodiment, the recombinant bacterial cell comprises at least one outer membrance nucleoside transporter, e.g., tsx, and at least one inner membrane nucleoside transporter, e.g., nupC and/or nupG. In one embodiment, said first nucleoside transporter is derived from a different organism than said second nucleoside transporter. In some embodiments, said first nucleoside transporter is derived from the same organism as said second nucleoside transporter. In some embodiments, said first nucleoside transporter imports the same nucleoside as said second nucleoside transporter. In other embodiment, said first nucleoside transporter imports a different nucleoside from said second nucleoside transporter. In some embodiments, said first nucleoside transporter is a wild-type nucleoside transporter and said second nucleoside transporter is a mutagenized version of said first nucleoside transporter. In some embodiments, the recombinant bacterial cells described herein comprise at least a third heterologous nucleoside transporter. In some embodiments, the recombinant bacterial cells described herein comprise at least four heterologous nucleoside transporters. In some embodiments, the recombinant bacterial cells described herein comprise at least five heterologous nucleoside transporters or more.

In one embodiment, the nucleoside transporter imports one nucleoside into the bacterial cell. In another embodiment, the nucleoside transporter imports two nucleosides into the bacterial cell. In yet another embodiment, the nucleoside transporter imports three nucleosides into the bacterial cell. In another embodiment, the nucleoside transporter imports four or more nucleosides into the cell. In one embodiment, the nucleoside transporter is an outer membrane nucleoside transporter. In one embodiment, the nucleoside transporter is an inner membrane nucleoside transporter. In one embodiment, the nucleoside transporter is an adenosine transporter. In another embodiment, the nucleoside transporter is an guanosine transporter. In another embodiment, the nucleoside transporter is an uridine transporter. In another embodiment, the amino acid transporter is a inosine transporter. In another embodiment, the amino acid transporter is a xanthosine transporter. In another embodiment, the amino acid transporter is a thymidine transporter. In one embodiment, the nucleoside transporter is an cytidine transporter.

In some embodiment, the recombinant bacterial cell comprising a heterologous gene encoding a nucleoside transporter, e.g., an adenosine transporter, may be used to treat a disease, condition, and/or symptom associated with cancer, e.g., a cancer described herein. In some embodiments, the recombinant bacterial cells described herein may be used to reduce, ameliorate, or eliminate one or more symptom(s) associated with a cancer.

For example, an important barrier to successful cancer immunotherapy is that tumors employ a number of mechanisms to facilitate immune escape, including the production of anti-inflammatory cytokines, the recruitment of regulatory immune subsets, and the production of immunosuppressive metabolites. One such immunosuppressive pathway is the production of extracellular adenosine, a potent immunosuppressive molecule, by CD73. The purinergic system regulates and refines immune cell functions, such as cell-to-cell interactions, cytokine and chemokine secretion, surface antigen shedding, intracellular pathogen removal, and generating reactive oxygen species. Extracellular ATP, released by damaged or dying cells and bacteria, promotes the recruitment of immune phagocytes and activates P2X7R, a coactivator of the NLRP3 inflammasome, which then triggers the production of proinflammatory cytokines, such as IL-1B and IL-18. The catabolism of extracellular ATP into ADP, AMP and adenosine is controlled by glycosylphosphatidylinositol (GPI-) anchored ectonucleotidases and membrane-bound kinases. CD39 (ecto-nucleoside triphosphate diphosphohydrolase 1, E-NTPDasc1) hydrolyzes ATP into AMP, which is then dephosphorylated into adenosine by CD73 (ecto-5′-nucleotidase, Ecto5′NTase). Thus, CD39 and CD73 act in concert to convert proinflammatory ATP into immunosuppressive adenosine. Notably, the activity of CD39 is reversible by the actions of NDP kinase and adenylate kinase, whereas the activity of CD73 is virtually irreversible. Thus, CD73 represents a crucial checkpoint in the conversion of an ATP-driven proinflammatory environment to an anti-inflammatory milieu induced by adenosine. Stated another way, CD73 negatively regulates the proinflammatory effects of extracellular adenosine triphosphate (ATP).

In the tumor setting, CD39 and CD73 generate increased adenosine levels characteristic of the tumor microenvironment. High expression and activity of CD39 and CD73 has been observed in several blood or solid tumors. In addition, CD39- and CD73-expressing cancer exosomes can also raise adenosine levels within the tumor microenvironment. The CD39/CD73 complex participates in the process of tumor immunoescape, by inhibiting the activation, clonal expansion, and homing of tumor-specific T cells (in particular, T helper and cytotoxic T cells), impairing tumor cell killing by cytolytic effector T lymphocytes, and inducing the suppressive capabilities of Treg and Th17 cells, and enhancing the conversion of type 1 macrophages into tumor-promoting type 2 macrophages (reviewed in Antonioli et al., Trends Mol Med. 2013 June; 19 (6): 355-367. CD39 and CD73 in immunity and inflammation). Myeloid-derived suppressor cells (MDSCs), also appear to promote tumor growth by a CD39-mediated mechanism.

Beside its immunoregulatory roles, the ectonucleotidase pathway contributes directly to the modulation of cancer cell growth, differentiation, invasion, migration, metastasis, and tumor angiogenesis. Agents targeting these enzymes show anti-tumor efficacy and a favorable tolerability profile in several murine models of malignancy (Anonioli et al., 2013). In some embodiments, the genetically engineered bacteria comprise a means for removing excess adenosine from the tumor microenvironment. Many bacteria scavenge low concentrations of nucleosides from the environment for synthesis of nucleotides and deoxynucleotides by salvage pathways of synthesis. Additionally, in Escherichia coli, nucleosides can be used as the sole source of nitrogen and carbon for growth (Neuhard J, Nygaard P. Biosynthesis and conversion of nucleotides, purines and pyrimidines. In: Neidhardt F C, Ingraham J L, Low K B, Magasanik B, Schaechter M, Umbarger H E, editors. Escherichia coli and Salmonella typhimurium: Cellular and molecular biology. Washington DC: ASM Press; 1987. pp. 445-473). Two evolutionarily unrelated cation-linked transporter families, the Concentrative Nucleoside Transporter (CNT) family and the Nucleoside:H+ Symporter (NHS) family, are responsible for nucleoside uptake (see e.g., Cabrita et al., Biochem. Cell Biol. Vol. 80, 2002. Molecular biology and regulation of nucleoside and nucleobase transporter proteins in eukaryotes and prokaryotes), the contents of which is herein incorporated by reference in its entirety. NupC and NupG, are the transporter family members in E. coli. Mutants defective in both the nupC and nupG genes cannot grow with nucleosides as a single carbon source. Both of these transporters are proton-linked but they differ in their selectivity. NupG is capable of transporting a wide range of nucleosides and deoxynucleosides; in contrast, NupC does not transport guanosine or deoxyguanosine. Homologs of NupG from E. coli are found in a wide range of eubacteria, including human gut pathogens such as Salmonella typhimurium, organisms associated with periodontal disease such as Porphyromonas gingivalis and Prevotella intermedia, and plant pathogens in the genus Erwinia (As described in Vaziri et al., Mol Membr Biol. 2013 March; 30 (1-2): 114-128. Use of molecular modelling to probe the mechanism of the nucleoside transporter NupG, the contents of which is herein incorporated by reference in its entirety). Putative bacterial transporters from the CNT superfamily and transporters from the NupG/XapB family include those listed herein. In addition, codB (GenBank P25525, Escherichia coli) was identified based on homology to a yeast transporter family termed the uracil/allantoin transpertor family (Cabrita et al., supra).

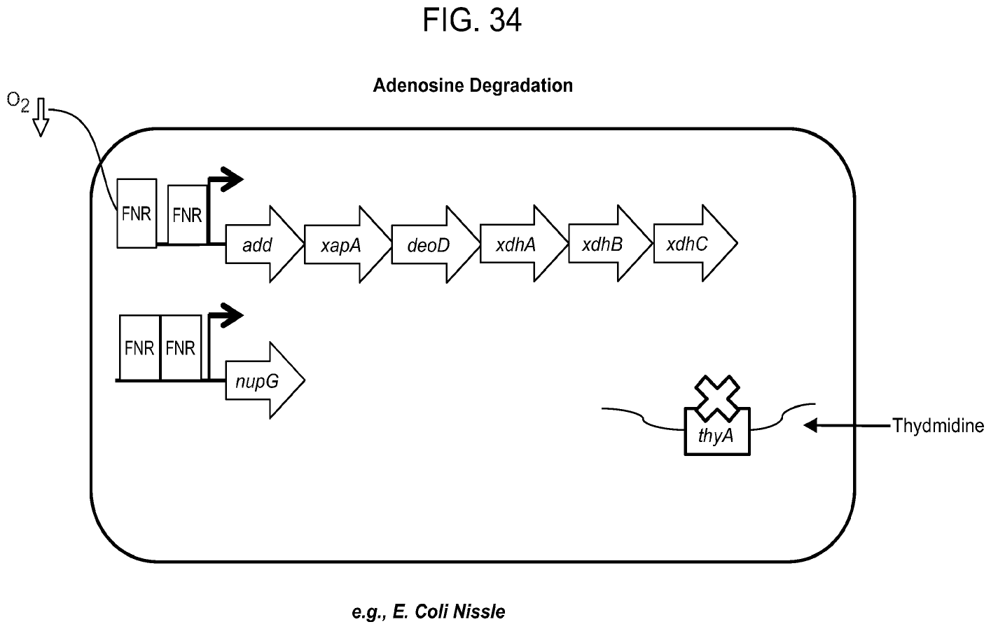

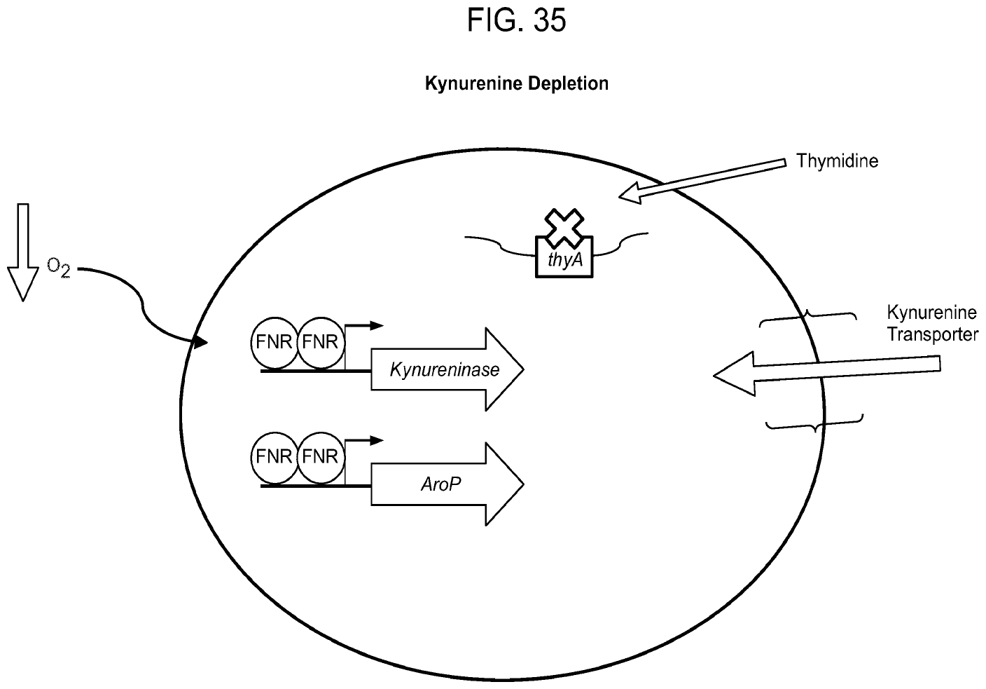

Thus, the genetically engineered bacteria comprise a means for metabolizing or degrading adenosine. In some embodiments, the genetically engineered bacteria comprise one or more gene sequences encoding one or more enzymes that are capable of converting adenosine to urate. In some embodiments, the genetically engineered bacteria comprise sequence(s) encoding add, xapA, deoD, xdhA, xdhB, and xdhC genes from E. Coli. In some embodiments, the genetically engineered bacteria or genetically engineered oncolytic virus further comprise a means for importing adenosine into the engineered bacteria from the tumor microenvironment. In some embodiments, the genetically engineered bacteria comprise sequence for encoding a nucleoside transporter. In some embodiments, the genetically engineered bacteria for encoding an adenosine transporter. In certain embodiments, genetically engineered bacteria for encoding E. coli Nucleoside Permease nupG or nupC. In some embodiments, the genetically engineered bacteria comprise sequence(s) encoding add, xapA, deoD, xdhA, xdhB, and xdhC genes from E. Coli and comprise sequence encoding a nucleoside or adenosine transporter. In some embodiments, the genetically engineered bacteria comprise sequence(s) encoding add, xapA, deoD, xdhA, xdhB, and xdhC genes from E. Coli and comprise sequence encoding nupG or nupC. An exemplary engineered bacteria is shown in FIG. 34.

### C. Kynurenine Transporters

The catabolism of the essential amino acid tryptophan is a central pathway maintaining the immunosuppressive microenvironment in many types of cancers. Tumor cells or myeloid cells in the tumor microenvironment express high levels of indoleamine-2,3-dioxygenase 1 (IDO1), which is the first and rate-limiting enzyme in the degradation of tryptophan. This enzymatic activity results in the depletion of tryptophan in the local microenvironment and subsequent inhibition of T cell responses, which results in immunosuppression (as T cells are particularly sensitive to low tryptophan levels). More recent preclinical studies suggest an alternative route of tryptophan degradation in tumors via the enzyme TRP-2,3-dioxygenase 2 (TDO). Thus, tumor cells may express and catabolize tryptophan via TDO instead of or in addition to IDO1.

In addition, several studies have proposed that immunosuppression by tryptophan degradation is not solely a consequence of lowering local tryptophan levels but also of accumulating high levels of tryptophan metabolites. Preclinical studies and analyses of human tumor tissue have demonstrated that T cell responses are inhibited by tryptophan metabolites, primarily by binding to the aryl hydrocarbon receptor (AHR), a cytoplasmic transcription factor. These studies show that binding of the tryptophan metabolite kynurenine to the aryl hydrocarbon receptor results in reprograming the differentiation of naïve CD4+ T-helper (Th) cells favoring a regulatory T cells phenotype (Treg) while suppressing the differentiation into interleukin-17 (IL-17)-producing Th (Th17) cells. Activation of the aryl hydrogen receptor also results in promoting a tolerogenic phenotype on dendritic cells. As discussed above, studies have shown that the binding of kynurenine to the aryl hydrocarbon receptor results in the production of regulatory T cells (Tregs). Thus, in some embodiments, the engineered microbe has a mechanism for importing (transporting) Kynurenine from the local environment into the cell. Thus, in some embodiments, the genetically engineered bacteria comprise gene sequence(s) encoding a kynureninase transporter. In some embodiments, the genetically engineered bacteria comprise one or more copies of aroP, tnaB or mtr gene.

In one embodiment, the recombinant bacterial cell of the invention comprises a heterologous gene encoding a substrate transporter, wherein the substrate transporter is a kynurenine transporter. In one embodiment, the kynurenine transporter transports kynurenine into the cell.

The uptake of kynurenine into bacterial cells is mediated by proteins well known to those of skill in the art. In one embodiment, the at least one gene encoding a transporter is a gene selected from the group consisting of mtr, aroP and tnaB. In one embodiment, the bacterial cell described herein has been genetically engineered to comprise at least one heterologous gene selected from the group consisting of mtr, aroP and tnaB. In one embodiment, the at least one gene encoding a kynurenine transporter is the Escherichia coli mtr gene. In one embodiment, the at least one gene encoding a kynurenine transporter is the Escherichia coli aroP gene. In one embodiment, the at least one gene encoding a kynurenine transporter is the Escherichia coli tnaB gene.

In some embodiments, the kynurenine transporter is encoded by a kynurenine transporter gene derived from a bacterial genus or species, including but not limited to, Escherichia, Corynebacterium, Sacharomyces, Escherichia coli, Saccharomyces cerevisiae or Corynebacterium glutamicum. In some embodiments, the bacterial species is Escherichia coli. In some embodiments, the bacterial species is Escherichia coli strain Nissle.

Assays for testing the activity of a kynurenine transporter, a functional variant of a kynurenine transporter, or a functional fragment of transporter of kynurenine are well known to one of ordinary skill in the art.

Kynurenine transporters may be expressed or modified in the bacteria in order to enhance kynurenine transport into the cell. Specifically, when the kynurenine transporter is expressed in the recombinant bacterial cells, the bacterial cells import more kynurenine(s) into the cell when the transporter is expressed than unmodified bacteria of the same bacterial subtype under the same conditions. In one embodiment, the bacterial cell comprises a heterologous gene encoding a kynurenine transporter. In one embodiment, the bacterial cell comprises a heterologous gene encoding a kynurenine transporter and a genetic modification that reduces export of a kynurenine, e.g., a genetic mutation in an exporter gene or promoter.

In one embodiment, the bacterial cell comprises at least one gene encoding a kynurenine transporter from a different organism, e.g., a different species of bacteria. In one embodiment, the bacterial cell comprises at least one native gene encoding a kynurenine transporter. In some embodiments, the at least one native gene encoding a kynurenine transporter is not modified. In another embodiment, the bacterial cell comprises more than one copy of at least one native gene encoding a kynurenine transporter. In yet another embodiment, the bacterial cell comprises a copy of at least one gene encoding a native kynurenine transporter, as well as at least one copy of at least one heterologous gene encoding a kynurenine transporter from a different bacterial species. In one embodiment, the bacterial cell comprises at least one, two, three, four, five, or six copies of the at least one heterologous gene encoding a kynurenine transporter. In one embodiment, the bacterial cell comprises multiple copies of the at least one heterologous gene encoding a kynurenine transporter.

In one embodiment, the recombinant bacterial cell comprises a heterologous gene encoding a kynurenine transporter, wherein said kynurenine transporter comprises a kynurenine sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the kynurenine sequence of a polypeptide encoded by a kynurenine transporter gene disclosed herein.

The present disclosure further comprises genes encoding functional fragments of a kynurenine transporter or functional variants of a kynurenine transporter. As used herein, the term “functional fragment thereof” or “functional variant thereof” of a kynurenine transporter relates to an element having qualitative biological activity in common with the wild-type kynurenine transporter from which the fragment or variant was derived. For example, a functional fragment or a functional variant of a mutated kynurenine transporter is one which retains essentially the same ability to import a kynurenine into the bacterial cell as does the kynurenine transporter protein from which the functional fragment or functional variant was derived. In one embodiment, the recombinant bacterial cell comprises at least one heterologous gene encoding a functional fragment of a kynurenine transporter. In another embodiment, the recombinant bacterial cell comprises a heterologous gene encoding a functional variant of a kynurenine transporter.

In one embodiment, the genes encoding the kynurenine transporter have been codon-optimized for use in the host organism, e.g., a bacterial cell disclosed herein. In one embodiment, the genes encoding the kynurenine transporter have been codon-optimized for use in Escherichia coli.

The present disclosure also encompasses genes encoding a kynurenine transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a kynurenine transporter is mutagenized; mutants exhibiting increased kynurenine import are selected; and the mutagenized at least one gene encoding a kynurenine transporter is isolated and inserted into the bacterial cell. In some embodiments, the at least one gene encoding a kynurenine transporter is mutagenized; mutants exhibiting decreased kynurenine import are selected; and the mutagenized at least one gene encoding a kynurenine transporter is isolated and inserted into the bacterial cell. The transporter modifications described herein may be present on a plasmid or chromosome.

In some embodiments, the bacterial cell comprises a heterologous gene encoding a kynurenine transporter operably linked to a promoter. In one embodiment, the at least one gene encoding a kynurenine transporter is directly operably linked to the promoter. In another embodiment, the at least one gene encoding a kynurenine transporter is indirectly operably linked to the promoter.

In one embodiment, the promoter is not operably linked with the at least one gene encoding a kynurenine transporter in nature. In some embodiments, the at least one gene encoding the kynurenine transporter is controlled by its native promoter. In some embodiments, the at least one gene encoding the kynurenine transporter is controlled by an inducible promoter. In some embodiments, the at least one gene encoding the kynurenine transporter is controlled by a promoter that is stronger than its native promoter. In some embodiments, the at least one gene encoding the kynurenine transporter is controlled by a constitutive promoter.

In another embodiment, the promoter is an inducible promoter. Inducible promoters are described in more detail infra.

In one embodiment, the at least one gene encoding a kynurenine transporter is located on a plasmid in the bacterial cell. In some embodiments, the plasmid is a high copy number plasmid. In some embodiments, the plasmid is a low copy number plasmid. In another embodiment, the at least one gene encoding a kynurenine transporter is located in the chromosome of the bacterial cell. In yet another embodiment, a native copy of the at least one gene encoding a kynurenine transporter is located in the chromosome of the bacterial cell, and a copy of at least one gene encoding a kynurenine transporter from a different species of bacteria is located on a plasmid in the bacterial cell. In yet another embodiment, a native copy of the at least one gene encoding a kynurenine transporter is located on a plasmid in the bacterial cell, and a copy of at least one gene encoding a kynurenine transporter from a different species of bacteria is located on a plasmid in the bacterial cell. In yet another embodiment, a native copy of the at least one gene encoding a kynurenine transporter is located in the chromosome of the bacterial cell, and a copy of the at least one gene encoding a kynurenine transporter from a different species of bacteria is located in the chromosome of the bacterial cell.

In some embodiments, the at least one native gene encoding the kynurenine transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the kynurenine transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the kynurenine transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the kynurenine transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In one embodiment, the mtr gene has at least about 80% identity with the sequence of SEQ ID NO:46. Accordingly, in one embodiment, the mtr gene has at least about 90% identity with the sequence of SEQ ID NO:46. Accordingly, in one embodiment, the mtr gene has at least about 95% identity with the sequence of SEQ ID NO:46. Accordingly, in one embodiment, the mtr gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 46. In another embodiment, the mtr gene comprises the sequence of SEQ ID NO:46. In yet another embodiment the mtr gene consists of the sequence of SEQ ID NO:46.

In one embodiment, the tnaB gene has at least about 80% identity with the sequence of SEQ ID NO:47. Accordingly, in one embodiment, the tnaB gene has at least about 90% identity with the sequence of SEQ ID NO:47. Accordingly, in one embodiment, the tnaB gene has at least about 95% identity with the sequence of SEQ ID NO:47. Accordingly, in one embodiment, the tnaB gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO: 47. In another embodiment, the tnaB gene comprises the sequence of SEQ ID NO:47. In yet another embodiment the tnaB gene consists of the sequence of SEQ ID NO:47.

In one embodiment, the aroP gene has at least about 80% identity with the sequence of SEQ ID NO:48. Accordingly, in one embodiment, the aroP gene has at least about 90% identity with the sequence of SEQ ID NO:48. Accordingly, in one embodiment, the aroP gene has at least about 95% identity with the sequence of SEQ ID NO:48. Accordingly, in one embodiment, the aroP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:48. In another embodiment, the aroP gene comprises the sequence of SEQ ID NO: 48. In yet another embodiment the aroP gene consists of the sequence of SEQ ID NO:48.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the kynurenine transporter is expressed in the recombinant bacterial cells, the bacterial cells import 10% more kynurenine into the bacterial cell when the transporter is expressed than unmodified bacteria of the same bacterial subtype under the same conditions. In another embodiment, when the kynurenine transporter is expressed in the recombinant bacterial cells, the bacterial cells import 20%, 30%, 40%, 50%, 60%, 70%, 80%, 90% or 100% more kynurenine, into the bacterial cell when the transporter is expressed than unmodified bacteria of the same bacterial subtype under the same conditions. In yet another embodiment, when the kynurenine transporter is expressed in the recombinant bacterial cells, the bacterial cells import two-fold more kynurenine into the cell when the transporter is expressed than unmodified bacteria of the same bacterial subtype under the same conditions. In yet another embodiment, when the kynurenine transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more kynurenine into the cell when the transporter is expressed than unmodified bacteria of the same bacterial subtype under the same conditions.

In one embodiment, the recombinant bacterial cells described herein comprise a first heterologous kynurenine transporter and a second heterologous kynurenine transporter. In one embodiment, said first kynurenine transporter is derived from a different organism than said second kynurenine transporter. In some embodiments, said first kynurenine transporter is derived from the same organism as said second kynurenine transporter. In some embodiments, said first kynurenine transporter imports the same kynurenine as said second kynurenine transporter. In other embodiment, said first kynurenine transporter imports a different kynurenine from said second kynurenine transporter. In some embodiments, said first kynurenine transporter is a wild-type kynurenine transporter and said second kynurenine transporter is a mutagenized version of said first kynurenine transporter. In some embodiments, the recombinant bacterial cells described herein comprise at least a third heterologous kynurenine transporter. In some embodiments, the recombinant bacterial cells described herein comprise at least four heterologous kynurenine transporters. In some embodiments, the recombinant bacterial cells described herein comprise at least five heterologous kynurenine transporters or more.

In some embodiment, the recombinant bacterial cell comprising a heterologous gene encoding a kynurenine transporter may be used to treat a disease, condition, and/or symptom associated with cancer, e.g., a cancer described herein. In some embodiments, the recombinant bacterial cells described herein may be used to reduce, ameliorate, or eliminate one or more symptom(s) associated with a cancer.

Means for optimizing kynurenine uptake are provided in the Example section.

### D. Prostaglandin E2 Transporters

Prostaglandin E2 (PGE2) is overproduced in many tumors, where it aids in cancer progression. PGE2 is a pleiotropic molecule involved in numerous biological processes, including angiogenesis, apoptosis, inflammation, and immune suppression. PGE2 is synthesized from arachidonic acid by cyclooxygenase 2 (COX-2). COX-2, converts arachidonic acid (AA) to prostaglandin endoperoxide H2 (PGH2). PHG2 is then converted to PHE2 by prostaglandin E synthase (PGES), of which there are three forms. PGE2 can be catabolized into biologically inactive 15-keto-PGs by 15-PGDH and carbonyl reductase or secreted by the transporter MRP4.

MDSCs are thought to play a key role in the PGE2 production in the tumor environment. Tumor derived factors induce COX2, PGES1, and MRP4 and downregulate the expression of 15-PGDH in MDSCs, and is associated with MDSC suppressive activity. Inhibition of PGE2 through COX-2 inhibitors show promise as cancer treatments, but systemic administration is associated with serious side effects, and in the case of the COX-2 inhibitor celecoxib, resistance to tumor prevention has been observed.

In addition to inhibition of PGE production, the degradation of PGE2 by 15-hydroxyprostaglandin dehydrogenase (15-PGDH) is another way to reduce PGE2 levels in tumors. A lack of prostaglandin dehydrogenase prevents catabolism of prostaglandin E2, which helps cancer cells both to evade the immune system and circumvent drug treatment. Recent studies have demonstrated that 15-PGDH delivered locally to the tumor microenvironment can effect an antitumor immune response. For example, injection of an adenovirus encoding 15-PGDH into mouse tumors comprising non-lymphocyte white blood cells expressing CD11b (which have increased PGE2 levels, higher COX-2 expression and significantly reduced expression of 15-PGDH as compared with cells from outside the tumor), resulted in significantly slowed tumor growth. These studies further showed that 15-PGDH expression was highest in tumor cells but also significant in tumor-associated CD11b cells, where it produced a four-fold reduction in PGE2 secretion. This was associated with reduced secretion of immunosuppressive cytokines by the CD11b cells which resulted in a switch in their fate, promoting their differentiation into dendritic cells. These studies show that overproduction of PGE2 in tumors contributes to immune evasion by preventing maturation of antigen-presenting cells, and that evasion can be overcome by enforced expression of 15-PGDH. (Eruslanov et al., Volume 88, November 2010 Journal of Leukocyte Biology; Tumor-mediated induction of myeloid-derived suppressor cells and M2-polarized macrophages by altering intracellular PGE2 catabolismin myeloid cells).

Other studies confirm the benefit of local PGE2 catabolismin cancer treatment. Celecoxib, a non-steroidal anti-inflammatory COX-2 inhibitor used to treat pain and inflammation, reduces the recurrence of colon adenomas but does not work in some patients who have low levels of 15-PGDH. These results correspond with studies which show that in mice, gene knockout of 15-PGDH confers near-complete resistance to the ability of celecoxib to prevent colon tumors. These and other studies highlight the potential importance of reducing PGE2 levels in cancer, either through inhibition of synthesis or promotion of catalysis or both.

In one embodiment, the recombinant bacterial cell of the invention comprises a heterologous gene encoding a substrate transporter, wherein the substrate transporter is a prostaglandin E2 (PGE2) transporter. In one embodiment, the PGE2 transporter transports PGE2 into the cell.

The uptake of PGE2 into bacterial cells is mediated by proteins well known to those of skill in the art.

In some embodiments, the PGE2 transporter is encoded by a PGE2 transporter gene derived from a bacterial genus or species, including but not limited to, Escherichia, Corynebacterium, Escherichia coli, Saccharomyces cerevisiae or Corynebacterium glutamicum. In some embodiments, the bacterial species is Escherichia coli. In some embodiments, the bacterial species is Escherichia coli strain Nissle.

Assays for testing the activity of a PGE2 transporter, a functional variant of a PGE2 transporter, or a functional fragment of transporter of PGE2 are well known to one of ordinary skill in the art. For example, import of PGE2 may be determined using the methods as described in, the entire contents of each of which are expressly incorporated by reference herein.

PGE2 transporters may be expressed or modified in the bacteria in order to enhance PGE2 transport into the cell. Specifically, when the PGE2 transporter is expressed in the recombinant bacterial cells, the bacterial cells import more PGE2 into the cell when the transporter is expressed than unmodified bacteria of the same bacterial subtype under the same conditions. In one embodiment, the bacterial cell comprises a heterologous gene encoding a PGE2 transporter. In one embodiment, the bacterial cell comprises a heterologous gene encoding a PGE2 transporter and a genetic modification that reduces export of a PGE2, e.g., a genetic mutation in an exporter gene or promoter.

In one embodiment, the bacterial cell comprises at least one gene encoding a PGE2 transporter from a different organism, e.g., a different species of bacteria. In one embodiment, the bacterial cell comprises at least one native gene encoding a PGE2 transporter. In some embodiments, the at least one native gene encoding a PGE2 transporter is not modified. In another embodiment, the bacterial cell comprises more than one copy of at least one native gene encoding a PGE2 transporter. In yet another embodiment, the bacterial cell comprises a copy of at least one gene encoding a native PGE2 transporter, as well as at least one copy of at least one heterologous gene encoding a PGE2 transporter from a different bacterial species. In one embodiment, the bacterial cell comprises at least one, two, three, four, five, or six copies of the at least one heterologous gene encoding a PGE2 transporter. In one embodiment, the bacterial cell comprises multiple copies of the at least one heterologous gene encoding a PGE2 transporter.

In one embodiment, the recombinant bacterial cell comprises a heterologous gene encoding a PGE2 transporter, wherein said PGE2 transporter comprises a PGE2 sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the PGE2 sequence of a polypeptide encoded by a PGE2 transporter gene disclosed herein.

The present disclosure further comprises genes encoding functional fragments of a PGE2 transporter or functional variants of a PGE2 transporter. As used herein, the term “functional fragment thereof” or “functional variant thereof” of a PGE2 transporter relates to an element having qualitative biological activity in common with the wild-type PGE2 transporter from which the fragment or variant was derived. For example, a functional fragment or a functional variant of a mutated PGE2 transporter is one which retains essentially the same ability to import PGE2 into the bacterial cell as does the PGE2 transporter protein from which the functional fragment or functional variant was derived. In one embodiment, the recombinant bacterial cell comprises at least one heterologous gene encoding a functional fragment of a PGE2 transporter. In another embodiment, the recombinant bacterial cell comprises a heterologous gene encoding a functional variant of a PGE2 transporter.

In one embodiment, the genes encoding the PGE2 transporter have been codon-optimized for use in the host organism, e.g., a bacterial cell disclosed herein. In one embodiment, the genes encoding the PGE2 transporter have been codon-optimized for use in Escherichia coli.

The present disclosure also encompasses genes encoding a PGE2 transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a PGE2 transporter is mutagenized; mutants exhibiting increased PGE2 import are selected; and the mutagenized at least one gene encoding a PGE2 transporter is isolated and inserted into the bacterial cell. In some embodiments, the at least one gene encoding a PGE2 transporter is mutagenized; mutants exhibiting decreased PGE2 import are selected; and the mutagenized at least one gene encoding a PGE2 transporter is isolated and inserted into the bacterial cell. The transporter modifications described herein may be present on a plasmid or chromosome.

In some embodiments, the bacterial cell comprises a heterologous gene encoding a PGE2 transporter operably linked to a promoter. In one embodiment, the at least one gene encoding a PGE2 transporter is directly operably linked to the promoter. In another embodiment, the at least one gene encoding a PGE2 transporter is indirectly operably linked to the promoter.

In one embodiment, the promoter is not operably linked with the at least one gene encoding a PGE2 transporter in nature. In some embodiments, the at least one gene encoding the PGE2 transporter is controlled by its native promoter. In some embodiments, the at least one gene encoding the PGE2 transporter is controlled by an inducible promoter. In some embodiments, the at least one gene encoding the PGE2 transporter is controlled by a promoter that is stronger than its native promoter. In some embodiments, the at least one gene encoding the PGE2 transporter is controlled by a constitutive promoter.

In another embodiment, the promoter is an inducible promoter. Inducible promoters are described in more detail infra.

In one embodiment, the at least one gene encoding a PGE2 transporter is located on a plasmid in the bacterial cell. In some embodiments, the plasmid is a high copy number plasmid. In some embodiments, the plasmid is a low copy number plasmid. In another embodiment, the at least one gene encoding a PGE2 transporter is located in the chromosome of the bacterial cell. In yet another embodiment, a native copy of the at least one gene encoding a PGE2 transporter is located in the chromosome of the bacterial cell, and a copy of at least one gene encoding a PGE2 transporter from a different species of bacteria is located on a plasmid in the bacterial cell. In yet another embodiment, a native copy of the at least one gene encoding a PGE2 transporter is located on a plasmid in the bacterial cell, and a copy of at least one gene encoding a PGE2 transporter from a different species of bacteria is located on a plasmid in the bacterial cell. In yet another embodiment, a native copy of the at least one gene encoding a PGE2 transporter is located in the chromosome of the bacterial cell, and a copy of the at least one gene encoding a PGE2 transporter from a different species of bacteria is located in the chromosome of the bacterial cell.

In some embodiments, the at least one native gene encoding the PGE2 transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the PGE2 transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the PGE2 transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the PGE2 transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the PGE2 transporter is expressed in the recombinant bacterial cells, the bacterial cells import 10% more PGE2 into the bacterial cell when the transporter is expressed than unmodified bacteria of the same bacterial subtype under the same conditions. In another embodiment, when the PGE2 transporter is expressed in the recombinant bacterial cells, the bacterial cells import 20%, 30%, 40%, 50%, 60%, 70%, 80%, 90% or 100% more PGE2, into the bacterial cell when the transporter is expressed than unmodified bacteria of the same bacterial subtype under the same conditions. In yet another embodiment, when the PGE2 transporter is expressed in the recombinant bacterial cells, the bacterial cells import two-fold more PGE2 into the cell when the transporter is expressed than unmodified bacteria of the same bacterial subtype under the same conditions. In yet another embodiment, when the PGE2 transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more PGE2 into the cell when the transporter is expressed than unmodified bacteria of the same bacterial subtype under the same conditions.

In one embodiment, the recombinant bacterial cells described herein comprise a first heterologous PGE2 transporter and a second heterologous PGE2 transporter. In one embodiment, said first PGE2 transporter is derived from a different organism than said second PGE2 transporter. In some embodiments, said first PGE2 transporter is derived from the same organism as said second PGE2 transporter. In some embodiments, said first PGE2 transporter is a wild-type PGE2 transporter and said second PGE2 transporter is a mutagenized version of said first PGE2 transporter. In some embodiments, the recombinant bacterial cells described herein comprise at least a third heterologous PGE2 transporter. In some embodiments, the recombinant bacterial cells described herein comprise at least four heterologous PGE2 transporters. In some embodiments, the recombinant bacterial cells described herein comprise at least five heterologous PGE2 transporters or more.

In some embodiment, the recombinant bacterial cell comprising a heterologous gene encoding a PGE2 transporter may be used to treat a disease, condition, and/or symptom associated with cancer, e.g., a cancer described herein. In some embodiments, the recombinant bacterial cells described herein may be used to reduce, ameliorate, or eliminate one or more symptom(s) associated with a cancer.

### E. Lactic Acid Transporters

The anti-cancer immune response is influenced by the environmental pH; an acidic pH has been shown to inhibit the function of immune cells. Lowering the environmental pH to 6.0-6.5, as can be found in tumour masses, has been reported to lead to loss of T-cell function of human and murine tumour-infiltrating lymphocytes (eg impairment of cytolytic activity and cytokine secretion); the T-cell function could be completely restored by buffering the pH at physiological values. The primary cause responsible for the acidic pH and pH-dependent T-cell function-suppressive effect in a tumour micro-environment has been identified as lactic acid (as reviewed in Chio et al., J Pathol. 2013 August; 230 (4): 350-355. Cancer-generated lactic acid: a regulatory, immunosuppressive metabolite?), the contents of which is herein incorporated by reference in its entirety. It has also been demonstrated that cancer-generated lactic acid and the resultant acidification of the micro-environment increase the expression of ARG1 in tumour-associated macrophages, characteristic of the M2 helper phenotype.

In some embodiments, the genetically engineered bacterium are able to import lactic acid from the tumor microenvironment. In one embodiment, the recombinant bacterial cell of the invention comprises a heterologous gene encoding a substrate transporter, wherein the substrate transporter is a lactic acid transporter. In one embodiment, the lactic acid transporter transports lactic acid into the cell.

The uptake of lactic acid into bacterial cells is mediated by proteins well known to those of skill in the art.

In some embodiments, the lactic acid transporter is encoded by a lactic acid transporter gene derived from a bacterial genus or species, including but not limited to, Escherichia, Corynebacterium, Escherichia coli, Saccharomyces cerevisiae or Corynebacterium glutamicum. In some embodiments, the bacterial species is Escherichia coli. In some embodiments, the bacterial species is Escherichia coli strain Nissle.

Assays for testing the activity of a lactic acid transporter, a functional variant of a lactic acid transporter, or a functional fragment of transporter of lactic acid are well known to one of ordinary skill in the art.

lactic acid transporters may be expressed or modified in the bacteria in order to enhance lactic acid transport into the cell. Specifically, when the lactic acid transporter is expressed in the recombinant bacterial cells, the bacterial cells import more lactic acid into the cell when the transporter is expressed than unmodified bacteria of the same bacterial subtype under the same conditions. In one embodiment, the bacterial cell comprises a heterologous gene encoding a lactic acid transporter. In one embodiment, the bacterial cell comprises a heterologous gene encoding a lactic acid transporter and a genetic modification that reduces export of a lactic acid, e.g., a genetic mutation in an exporter gene or promoter.

In one embodiment, the bacterial cell comprises at least one gene encoding a lactic acid transporter from a different organism, e.g., a different species of bacteria. In one embodiment, the bacterial cell comprises at least one native gene encoding a lactic acid transporter. In some embodiments, the at least one native gene encoding a lactic acid transporter is not modified. In another embodiment, the bacterial cell comprises more than one copy of at least one native gene encoding a lactic acid transporter. In yet another embodiment, the bacterial cell comprises a copy of at least one gene encoding a native lactic acid transporter, as well as at least one copy of at least one heterologous gene encoding a lactic acid transporter from a different bacterial species. In one embodiment, the bacterial cell comprises at least one, two, three, four, five, or six copies of the at least one heterologous gene encoding a lactic acid transporter. In one embodiment, the bacterial cell comprises multiple copies of the at least one heterologous gene encoding a lactic acid transporter.

In one embodiment, the recombinant bacterial cell comprises a heterologous gene encoding a lactic acid transporter, wherein said lactic acid transporter comprises a lactic acid sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the lactic acid sequence of a polypeptide encoded by a lactic acid transporter gene disclosed herein.

The present disclosure further comprises genes encoding functional fragments of a lactic acid transporter or functional variants of a lactic acid transporter. As used herein, the term “functional fragment thereof” or “functional variant thereof” of a lactic acid transporter relates to an element having qualitative biological activity in common with the wild-type lactic acid transporter from which the fragment or variant was derived. For example, a functional fragment or a functional variant of a mutated lactic acid transporter is one which retains essentially the same ability to import lactic acid into the bacterial cell as does the lactic acid transporter protein from which the functional fragment or functional variant was derived. In one embodiment, the recombinant bacterial cell comprises at least one heterologous gene encoding a functional fragment of a lactic acid transporter. In another embodiment, the recombinant bacterial cell comprises a heterologous gene encoding a functional variant of a lactic acid transporter.

In one embodiment, the genes encoding the lactic acid transporter have been codon-optimized for use in the host organism, e.g., a bacterial cell disclosed herein. In one embodiment, the genes encoding the lactic acid transporter have been codon-optimized for use in Escherichia coli.

The present disclosure also encompasses genes encoding a lactic acid transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a lactic acid transporter is mutagenized; mutants exhibiting increased lactic acid import are selected; and the mutagenized at least one gene encoding a lactic acid transporter is isolated and inserted into the bacterial cell. In some embodiments, the at least one gene encoding a lactic acid transporter is mutagenized; mutants exhibiting decreased lactic acid import are selected; and the mutagenized at least one gene encoding a lactic acid transporter is isolated and inserted into the bacterial cell. The transporter modifications described herein may be present on a plasmid or chromosome.

In some embodiments, the bacterial cell comprises a heterologous gene encoding a lactic acid transporter operably linked to a promoter. In one embodiment, the at least one gene encoding a lactic acid transporter is directly operably linked to the promoter. In another embodiment, the at least one gene encoding a lactic acid transporter is indirectly operably linked to the promoter.

In one embodiment, the promoter is not operably linked with the at least one gene encoding a lactic acid transporter in nature. In some embodiments, the at least one gene encoding the lactic acid transporter is controlled by its native promoter. In some embodiments, the at least one gene encoding the lactic acid transporter is controlled by an inducible promoter. In some embodiments, the at least one gene encoding the lactic acid transporter is controlled by a promoter that is stronger than its native promoter. In some embodiments, the at least one gene encoding the lactic acid transporter is controlled by a constitutive promoter.

In another embodiment, the promoter is an inducible promoter. Inducible promoters are described in more detail infra.

In one embodiment, the at least one gene encoding a lactic acid transporter is located on a plasmid in the bacterial cell. In some embodiments, the plasmid is a high copy number plasmid. In some embodiments, the plasmid is a low copy number plasmid. In another embodiment, the at least one gene encoding a lactic acid transporter is located in the chromosome of the bacterial cell. In yet another embodiment, a native copy of the at least one gene encoding a lactic acid transporter is located in the chromosome of the bacterial cell, and a copy of at least one gene encoding a lactic acid transporter from a different species of bacteria is located on a plasmid in the bacterial cell. In yet another embodiment, a native copy of the at least one gene encoding a lactic acid transporter is located on a plasmid in the bacterial cell, and a copy of at least one gene encoding a lactic acid transporter from a different species of bacteria is located on a plasmid in the bacterial cell. In yet another embodiment, a native copy of the at least one gene encoding a lactic acid transporter is located in the chromosome of the bacterial cell, and a copy of the at least one gene encoding a lactic acid transporter from a different species of bacteria is located in the chromosome of the bacterial cell.

In some embodiments, the at least one native gene encoding the lactic acid transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the lactic acid transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the lactic acid transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the lactic acid transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the lactic acid transporter is expressed in the recombinant bacterial cells, the bacterial cells import 10% more lactic acid into the bacterial cell when the transporter is expressed than unmodified bacteria of the same bacterial subtype under the same conditions. In another embodiment, when the lactic acid transporter is expressed in the recombinant bacterial cells, the bacterial cells import 20%, 30%, 40%, 50%, 60%, 70%, 80%, 90% or 100% more lactic acid, into the bacterial cell when the transporter is expressed than unmodified bacteria of the same bacterial subtype under the same conditions. In yet another embodiment, when the lactic acid transporter is expressed in the recombinant bacterial cells, the bacterial cells import two-fold more lactic acid into the cell when the transporter is expressed than unmodified bacteria of the same bacterial subtype under the same conditions. In yet another embodiment, when the lactic acid transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more lactic acid into the cell when the transporter is expressed than unmodified bacteria of the same bacterial subtype under the same conditions.

In one embodiment, the recombinant bacterial cells described herein comprise a first heterologous lactic acid transporter and a second heterologous lactic acid transporter. In one embodiment, said first lactic acid transporter is derived from a different organism than said second lactic acid transporter. In some embodiments, said first lactic acid transporter is derived from the same organism as said second lactic acid transporter. In some embodiments, said first lactic acid transporter is a wild-type lactic acid transporter and said second lactic acid transporter is a mutagenized version of said first lactic acid transporter. In some embodiments, the recombinant bacterial cells described herein comprise at least a third heterologous lactic acid transporter. In some embodiments, the recombinant bacterial cells described herein comprise at least four heterologous lactic acid transporters. In some embodiments, the recombinant bacterial cells described herein comprise at least five heterologous lactic acid transporters or more.

In some embodiment, the recombinant bacterial cell comprising a heterologous gene encoding a lactic acid transporter may be used to treat a disease, condition, and/or symptom associated with cancer, e.g., a cancer described herein. In some embodiments, the recombinant bacterial cells described herein may be used to reduce, ameliorate, or eliminate one or more symptom(s) associated with a cancer.

### E. Propionate Transporters

In one embodiment, the recombinant bacterial cell of the invention comprises a heterologous gene encoding a substrate transporter, wherein the substrate transporter is a propionate transporter. In one embodiment, the propionate transporter transports propionate into the cell.

The uptake of propionate into bacterial cells typically occurs via passive diffusion (see, for example, Kell et al., 1981, Biochem. Biophys. Res. Commun., 9981-8). However, the active import of propionate is also mediated by proteins well known to those of skill in the art. For example, a bacterial transport system for the update of propionate in Corynebacterium glutamicum named MctC (monocarboxylic acid transporter) is known (see, for example, Jolkver et al. (2009) J. Bacteriol. 191 (3): 940-8). The putP_6 propionate transporter from Virgibacillus species (UniProt A0A024QGU1) has also been identified.

Propionate transporters, may be expressed or modified in the bacteria of the invention in order to enhance propionate transport into the cell. Specifically, when the propionate transporter is expressed in the recombinant bacterial cells of the invention, the bacterial cells import more propionate into the cell when the transporter is expressed than unmodified bacteria of the same bacterial subtype under the same conditions. Thus, the genetically engineered bacteria comprising a heterologous gene encoding an propionate transporter may be used to import propionate into the bacteria and can be used to treat diseases associated with the catabolism of propionate, such as organic acidurias (including PA and MMA) and vitamin B12 deficiencies. In one embodiment, the bacterial cell of the invention comprises a heterologous gene encoding an propionate transporter.

The uptake of propionate into bacterial cells is mediated by proteins well known to those of skill in the art. In one embodiment, the at least one gene encoding a propionate transporter is a gene selected from the group consisting of metC, PutP_6, mctB, mctC, dip0780, dip0791, ce0909, ce0910, ce1091, ce1092, sco1822, sco1823, sco1218, sco1219, ce1091, sco5827, m_5160, m_5161, m_5165, m_5166, nfa 17930, nfa 17940, nfa 17950, nfa 17960, actP, yjcH, ywcB, and ywcA. In one embodiment, the bacterial cell described herein has been genetically engineered to comprise at least one heterologous gene selected from the group consisting of metC, PutP_6, mctB, mctC, dip0780, dip0791, ce0909, ce0910, ce1091, ce1092, sco1822, sco1823, sco1218, sco1219, ce1091, sco5827, m_5160, m_5161, m_5165, m_5166, nfa 17930, nfa 17940, nfa 17950, nfa 17960, actP, yjcH, ywcB, and ywcA. In one embodiment, the at least one gene encoding a propionate transporter is the metC gene. In one embodiment, the at least one gene encoding a propionate transporter is the putP_6 gene.

In some embodiments, the propionate transporter is encoded by a propionate transporter gene derived from a bacterial genus or species, including but not limited to, Bacillus, Campylobacter, Clostridium, Corynebacterium, Escherichia, Lactobacillus, Mycobacterium, Pseudomonas, Salmonella, Staphylococcus, Streptomyces, Bacillus subtilis, Campylobacter jejuni, Clostridium perfringens, Escherichia coli, Lactobacillus delbrueckii, Mycobacterium smegmatis, Nocardia farcinica, Pseudomonas aeruginosa, Salmonella typhimurium, Virgibacillus, or Staphylococcus aureus. In some embodiments, the propionate transporter gene is derived from Virgibacillus. In some embodiments, the propionate transporter gene is derived from Corynebacterium. In one embodiment, the propionate transporter gene is derived from Corynebacterium glutamicum. In another embodiment, the propionate transporter gene is derived from Corynebacterium diphtheria. In another embodiment, the propionate transporter gene is derived from Corynebacterium efficiens. In another embodiment, the propionate transporter gene is derived from Streptomyces coelicolor. In another embodiment, the propionate transporter gene is derived from Mycobacterium smegmatis. In another embodiment, the propionate transporter gene is derived from Nocardia farcinica. In another embodiment, the propionate transporter gene is derived from E. coli. In another embodiment, the propionate transporter gene is derived from B. subtilis. In some embodiments, the bacterial species is Escherichia coli. In some embodiments, the bacterial species is Escherichia coli strain Nissle.

Assays for testing the activity of a propionate transporter, a functional variant of a propionate transporter, or a functional fragment of transporter of propionate are well known to one of ordinary skill in the art. For example, propionate import can be assessed by expressing the protein, functional variant, or fragment thereof, in a recombinant bacterial cell that lacks an endogenous propionate transporter. Propionate import can also be assessed using mass spectrometry. Propionate import can also be expressed using gas chromatography. For example, samples can be injected into a Perkin Elmer Autosystem XL Gas Chromatograph containing a Supelco packed column, and the analysis can be performed according to manufacturing instructions (see, for example, Supelco I (1998) Analyzing fatty acids by packed column gas chromatography, Bulletin 856B: 2014). Alternatively, samples can be analyzed for propionate import using high-pressure liquid chromatography (HPLC). For example, a computer-controlled Waters HPLC system equipped with a model 600 quaternary solvent delivery system, and a model 996 photodiode array detector, and components of the sample can be resolved with an Aminex HPX-87H (300 by 7.8 mm) organic acid analysis column (Bio-Rad Laboratories) (see, for example, Palacios et al., 2003, J. Bacteriol., 185 (9): 2802-2810).

Propionate transporters may be expressed or modified in the bacteria in order to enhance propionate transport into the cell. Specifically, when the propionate transporter is expressed in the recombinant bacterial cells, the bacterial cells import more propionate into the cell when the transporter is expressed than unmodified bacteria of the same bacterial subtype under the same conditions. In one embodiment, the bacterial cell comprises a heterologous gene encoding a propionate transporter. In one embodiment, the bacterial cell comprises a heterologous gene encoding a propionate transporter and a genetic modification that reduces export of a propionate, e.g., a genetic mutation in an exporter gene or promoter.

In one embodiment, the bacterial cell comprises at least one gene encoding a propionate transporter from a different organism, e.g., a different species of bacteria. In one embodiment, the bacterial cell comprises at least one native gene encoding a propionate transporter. In some embodiments, the at least one native gene encoding a propionate transporter is not modified. In another embodiment, the bacterial cell comprises more than one copy of at least one native gene encoding a propionate transporter. In yet another embodiment, the bacterial cell comprises a copy of at least one gene encoding a native propionate transporter, as well as at least one copy of at least one heterologous gene encoding a propionate transporter from a different bacterial species. In one embodiment, the bacterial cell comprises at least one, two, three, four, five, or six copies of the at least one heterologous gene encoding a propionate transporter. In one embodiment, the bacterial cell comprises multiple copies of the at least one heterologous gene encoding a propionate transporter.

In one embodiment, the recombinant bacterial cell comprises a heterologous gene encoding a propionate transporter, wherein said propionate transporter comprises a propionate sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the propionate sequence of a polypeptide encoded by a propionate transporter gene disclosed herein.

The present disclosure further comprises genes encoding functional fragments of a propionate transporter or functional variants of a propionate transporter. As used herein, the term “functional fragment thereof” or “functional variant thereof” of a propionate transporter relates to an element having qualitative biological activity in common with the wild-type propionate transporter from which the fragment or variant was derived. For example, a functional fragment or a functional variant of a mutated propionate transporter is one which retains essentially the same ability to import propionate into the bacterial cell as does the propionate transporter protein from which the functional fragment or functional variant was derived. In one embodiment, the recombinant bacterial cell comprises at least one heterologous gene encoding a functional fragment of a propionate transporter. In another embodiment, the recombinant bacterial cell comprises a heterologous gene encoding a functional variant of a propionate transporter.

In one embodiment, the genes encoding the propionate transporter have been codon-optimized for use in the host organism, e.g., a bacterial cell disclosed herein. In one embodiment, the genes encoding the propionate transporter have been codon-optimized for use in Escherichia coli.

The present disclosure also encompasses genes encoding a propionate transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a propionate transporter is mutagenized; mutants exhibiting increased propionate import are selected; and the mutagenized at least one gene encoding a propionate transporter is isolated and inserted into the bacterial cell. In some embodiments, the at least one gene encoding a propionate transporter is mutagenized; mutants exhibiting decreased propionate import are selected; and the mutagenized at least one gene encoding a propionate transporter is isolated and inserted into the bacterial cell. The transporter modifications described herein may be present on a plasmid or chromosome.

In some embodiments, the bacterial cell comprises a heterologous gene encoding a propionate transporter operably linked to a promoter. In one embodiment, the at least one gene encoding a propionate transporter is directly operably linked to the promoter. In another embodiment, the at least one gene encoding a propionate transporter is indirectly operably linked to the promoter.

In one embodiment, the promoter is not operably linked with the at least one gene encoding a propionate transporter in nature. In some embodiments, the at least one gene encoding the propionate transporter is controlled by its native promoter. In some embodiments, the at least one gene encoding the propionate transporter is controlled by an inducible promoter. In some embodiments, the at least one gene encoding the propionate transporter is controlled by a promoter that is stronger than its native promoter. In some embodiments, the at least one gene encoding the propionate transporter is controlled by a constitutive promoter.

In another embodiment, the promoter is an inducible promoter. Inducible promoters are described in more detail infra.

In one embodiment, the at least one gene encoding a propionate transporter is located on a plasmid in the bacterial cell. In some embodiments, the plasmid is a high copy number plasmid. In some embodiments, the plasmid is a low copy number plasmid. In another embodiment, the at least one gene encoding a propionate transporter is located in the chromosome of the bacterial cell. In yet another embodiment, a native copy of the at least one gene encoding a propionate transporter is located in the chromosome of the bacterial cell, and a copy of at least one gene encoding a propionate transporter from a different species of bacteria is located on a plasmid in the bacterial cell. In yet another embodiment, a native copy of the at least one gene encoding a propionate transporter is located on a plasmid in the bacterial cell, and a copy of at least one gene encoding a propionate transporter from a different species of bacteria is located on a plasmid in the bacterial cell. In yet another embodiment, a native copy of the at least one gene encoding a propionate transporter is located in the chromosome of the bacterial cell, and a copy of the at least one gene encoding a propionate transporter from a different species of bacteria is located in the chromosome of the bacterial cell.

In some embodiments, the at least one native gene encoding the propionate transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the propionate transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the propionate transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the propionate transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In one embodiment, the propionate transporter is MctC. In one embodiment, the mctC gene has at least about 80% identity to SEQ ID NO:129. Accordingly, in one embodiment, the mctC gene has at least about 90% identity to SEQ ID NO:129. Accordingly, in one embodiment, the mctC gene has at least about 95% identity to SEQ ID NO:12. Accordingly, in one embodiment, the mctC gene has at least about 70%, 75%, 80%, 85%, 86%, 87%, 88%, 89%, 90%, 91%, 92%, 93%, 94%, 95%, 96%, 97%, 98%, or 99% identity to SEQ ID NO: 129. In another embodiment, the mctC gene comprises the sequence of SEQ ID NO: 129. In yet another embodiment the mctC gene consists of the sequence of SEQ ID NO: 129.

In another embodiment, the propionate transporter is PutP_6. In one embodiment, the putP_6 gene has at least about 80% identity to SEQ ID NO:130. Accordingly, in one embodiment, the putP_6 gene has at least about 90% identity to SEQ ID NO: 130. Accordingly, in one embodiment, the putP_6 gene has at least about 95% identity to SEQ ID NO:130. Accordingly, in one embodiment, the putP_6 gene has at least about 70%, 75%, 80%, 85%, 86%, 87%, 88%, 89%, 90%, 91%, 92%, 93%, 94%, 95%, 96%, 97%, 98%, or 99% identity to SEQ ID NO:130. In another embodiment, the putP_6 gene comprises the sequence of SEQ ID NO:130. In yet another embodiment the putP_6 gene consists of the sequence of SEQ ID NO:130.

Other propionate transporter genes are known to those of ordinary skill in the art. Sec, for example, Jolker et al., J. Bacteriol., 2009, 191 (3): 940-948. In one embodiment, the propionate transporter comprises the mctBC genes from C. glutamicum. In another embodiment, the propionate transporter comprises the dip0780 and dip0791 genes from C. diphtheria. In another embodiment, the propionate transporter comprises the ce0909 and ce0910 genes from C. efficiens. In another embodiment, the propionate transporter comprises the ce1091 and ce1092 genes from C. efficiens. In another embodiment, the propionate transporter comprises the sco1822 and sco1823 genes from S. coelicolor. In another embodiment, the propionate transporter comprises the sco1218 and sco1219 genes from S. coelicolor. In another embodiment, the propionate transporter comprises the ce1091 and sco5827 genes from S. coelicolor. In another embodiment, the propionate transporter comprises the m_5160, m_5161, m_5165, and m_5166 genes from M. smegmatis. In another embodiment, the propionate transporter comprises the nfa 17930, nfa 17940, nfa 17950, and nfa 17960 genes from N. farcinica. In another embodiment, the propionate transporter comprises the actP and yjcH genes from E. coli. In another embodiment, the propionate transporter comprises the ywcB and ywcA genes from B. subtilis.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the propionate transporter is expressed in the recombinant bacterial cells, the bacterial cells import 10% more propionate into the bacterial cell when the transporter is expressed than unmodified bacteria of the same bacterial subtype under the same conditions. In another embodiment, when the propionate transporter is expressed in the recombinant bacterial cells, the bacterial cells import 20%, 30%, 40%, 50%, 60%, 70%, 80%, 90% or 100% more propionate, into the bacterial cell when the transporter is expressed than unmodified bacteria of the same bacterial subtype under the same conditions. In yet another embodiment, when the propionate transporter is expressed in the recombinant bacterial cells, the bacterial cells import two-fold more propionate into the cell when the transporter is expressed than unmodified bacteria of the same bacterial subtype under the same conditions. In yet another embodiment, when the propionate transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more propionate into the cell when the transporter is expressed than unmodified bacteria of the same bacterial subtype under the same conditions.

In one embodiment, the recombinant bacterial cells described herein comprise a first heterologous propionate transporter and a second heterologous propionate transporter. In one embodiment, said first propionate transporter is derived from a different organism than said second propionate transporter. In some embodiments, said first propionate transporter is derived from the same organism as said second propionate transporter. In some embodiments, said first propionate transporter is a wild-type propionate transporter and said second propionate transporter is a mutagenized version of said first propionate transporter. In some embodiments, the recombinant bacterial cells described herein comprise at least a third heterologous propionate transporter. In some embodiments, the recombinant bacterial cells described herein comprise at least four heterologous propionate transporters. In some embodiments, the recombinant bacterial cells described herein comprise at least five heterologous propionate transporters or more.

In some embodiment, the recombinant bacterial cell comprising a heterologous gene encoding an propionate transporter may be used to treat a disease, condition, and/or symptom associated with the catabolism of propionate in a subject. In some embodiments, the recombinant bacterial cells described herein may be used to reduce, ameliorate, or eliminate one or more symptom(s) associated with these diseases or disorders. In one embodiment, the disorder associated with the catabolism of propionate is a metabolic disorder involving the abnormal catabolism of propionate. Metabolic diseases associated with abnormal catabolism of propionate include propionic acidemia (PA) and methylmalonic acidemia (MMA), as well as severe nutritional vitamin B12 deficiencies. In one embodiment, the disease associated with abnormal catabolism of propionate is propionic acidemia. In one embodiment, the disease associated with abnormal catabolism of propionate is methylmalonic acidemia. In another embodiment, the disease associated with abnormal catabolism of propionate is a vitamin B12 deficiency.

### G. Bile Salt Acid Transporters

In one embodiment, the recombinant bacterial cell of the invention comprises a heterologous gene encoding a substrate transporter, wherein the substrate transporter is a bile salt transporter. In one embodiment, the bile salt transporter transports bile salt into the cell.

The uptake of bile salt into bacterial cells is mediated by proteins well known to those of skill in the art. For example, the uptake of bile salts into the Lactobacillus and Bifidobacterium has been found to occur via the bile salt transporters CbsT1 and CbsT2 (see, e.g., Elkins et al., Microbiology, 147 (Pt. 12): 3403-3412 (2001), the entire contents of which are expressly incorporated herein by reference). Other proteins that mediate the import of bile salts into cells are well known to those of skill in the art.

In one embodiment, the at least one gene encoding a bile salt transporter is a cbsT1 or a cbsT2 gene. In one embodiment, the bacterial cell described herein has been genetically engineered to comprise at least one heterologous gene selected from a cbsT1 or a cbsT2 gene. In one embodiment, the at least one gene encoding a bile salt transporter is the cbsT1 gene. In one embodiment, the at least one gene encoding a bile salt transporter is the cbsT2 gene. In one embodiment, the bile acid transporter is the bile acid sodium symporter ASBT_{NM} (NMB0705 gene of Neisseria meningitides).

In some embodiments, the bile salt transporter is encoded by a bile salt transporter gene derived from a bacterial genus or species, including but not limited to, Lactobacillus, for example, Lactobacillus johnsonni (e.g., Lactobacillus johnsonni strain 100-100). In some embodiments, the bacterial species is Escherichia coli. In some embodiments, the bacterial species is Escherichia coli strain Nissle.

Assays for testing the activity of an transporter of a bile salt, a functional variant of an transporter of a bile salt, or a functional fragment of an transporter of a bile salt are well known to one of ordinary skill in the art. For example, bile salt import can be assessed as described in Elkins et al. (2001) Microbiology, 147:3403-3412, the entire contents of which are expressly incorporated herein by reference.

Bile salt transporters may be expressed or modified in the bacteria in order to enhance bile salt transport into the cell. Specifically, when the bile salt transporter is expressed in the recombinant bacterial cells, the bacterial cells import more bile salt into the cell when the transporter is expressed than unmodified bacteria of the same bacterial subtype under the same conditions. In one embodiment, the bacterial cell comprises a heterologous gene encoding a bile salt transporter. In one embodiment, the bacterial cell comprises a heterologous gene encoding a bile salt transporter and a genetic modification that reduces export of a bile salt, e.g., a genetic mutation in an exporter gene or promoter.

In one embodiment, the bacterial cell comprises at least one gene encoding a bile salt transporter from a different organism, e.g., a different species of bacteria. In one embodiment, the bacterial cell comprises at least one native gene encoding a bile salt transporter. In some embodiments, the at least one native gene encoding a bile salt transporter is not modified. In another embodiment, the bacterial cell comprises more than one copy of at least one native gene encoding a bile salt transporter. In yet another embodiment, the bacterial cell comprises a copy of at least one gene encoding a native bile salt transporter, as well as at least one copy of at least one heterologous gene encoding a bile salt transporter from a different bacterial species. In one embodiment, the bacterial cell comprises at least one, two, three, four, five, or six copies of the at least one heterologous gene encoding a bile salt transporter. In one embodiment, the bacterial cell comprises multiple copies of the at least one heterologous gene encoding a bile salt transporter.

In one embodiment, the recombinant bacterial cell comprises a heterologous gene encoding a bile salt transporter, wherein said bile salt transporter comprises a bile salt sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the bile salt sequence of a polypeptide encoded by a bile salt transporter gene disclosed herein.

The present disclosure further comprises genes encoding functional fragments of a bile salt transporter or functional variants of a bile salt transporter. As used herein, the term “functional fragment thereof” or “functional variant thereof” of a bile salt transporter relates to an element having qualitative biological activity in common with the wild-type bile salt transporter from which the fragment or variant was derived. For example, a functional fragment or a functional variant of a mutated bile salt transporter is one which retains essentially the same ability to import bile salt into the bacterial cell as does the bile salt transporter protein from which the functional fragment or functional variant was derived. In one embodiment, the recombinant bacterial cell comprises at least one heterologous gene encoding a functional fragment of a bile salt transporter. In another embodiment, the recombinant bacterial cell comprises a heterologous gene encoding a functional variant of a bile salt transporter.

In one embodiment, the genes encoding the bile salt transporter have been codon-optimized for use in the host organism, e.g., a bacterial cell disclosed herein. In one embodiment, the genes encoding the bile salt transporter have been codon-optimized for use in Escherichia coli.

The present disclosure also encompasses genes encoding a bile salt transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a bile salt transporter is mutagenized; mutants exhibiting increased bile salt import are selected; and the mutagenized at least one gene encoding a bile salt transporter is isolated and inserted into the bacterial cell. In some embodiments, the at least one gene encoding a bile salt transporter is mutagenized; mutants exhibiting decreased bile salt import are selected; and the mutagenized at least one gene encoding a bile salt transporter is isolated and inserted into the bacterial cell. The transporter modifications described herein may be present on a plasmid or chromosome.

In some embodiments, the bacterial cell comprises a heterologous gene encoding a bile salt transporter operably linked to a promoter. In one embodiment, the at least one gene encoding a bile salt transporter is directly operably linked to the promoter. In another embodiment, the at least one gene encoding a bile salt transporter is indirectly operably linked to the promoter.

In one embodiment, the promoter is not operably linked with the at least one gene encoding a bile salt transporter in nature. In some embodiments, the at least one gene encoding the bile salt transporter is controlled by its native promoter. In some embodiments, the at least one gene encoding the bile salt transporter is controlled by an inducible promoter. In some embodiments, the at least one gene encoding the bile salt transporter is controlled by a promoter that is stronger than its native promoter. In some embodiments, the at least one gene encoding the bile salt transporter is controlled by a constitutive promoter.

In another embodiment, the promoter is an inducible promoter. Inducible promoters are described in more detail infra.

In one embodiment, the at least one gene encoding a bile salt transporter is located on a plasmid in the bacterial cell. In some embodiments, the plasmid is a high copy number plasmid. In some embodiments, the plasmid is a low copy number plasmid. In another embodiment, the at least one gene encoding a bile salt transporter is located in the chromosome of the bacterial cell. In yet another embodiment, a native copy of the at least one gene encoding a bile salt transporter is located in the chromosome of the bacterial cell, and a copy of at least one gene encoding a bile salt transporter from a different species of bacteria is located on a plasmid in the bacterial cell. In yet another embodiment, a native copy of the at least one gene encoding a bile salt transporter is located on a plasmid in the bacterial cell, and a copy of at least one gene encoding a bile salt transporter from a different species of bacteria is located on a plasmid in the bacterial cell. In yet another embodiment, a native copy of the at least one gene encoding a bile salt transporter is located in the chromosome of the bacterial cell, and a copy of the at least one gene encoding a bile salt transporter from a different species of bacteria is located in the chromosome of the bacterial cell.

In some embodiments, the at least one native gene encoding the bile salt transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the bile salt transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the bile salt transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the bile salt transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In one embodiment, the bile salt transporter is the bile salt transporter CbsT1. In one embodiment, the cbsT1 gene has at least about 80% identity to SEQ ID NO:131. Accordingly, in one embodiment, the cbsT1 gene has at least about 90% identity to SEQ ID NO: 131. Accordingly, in one embodiment, the cbsT1 gene has at least about 95% identity to SEQ ID NO:131. Accordingly, in one embodiment, the cbsT1 gene has at least about 70%, 75%, 80%, 85%, 86%, 87%, 88%, 89%, 90%, 91%, 92%, 93%, 94%, 95%, 96%, 97%, 98%, or 99% identity to SEQ ID NO:131. In another embodiment, the cbsT1 gene comprises the sequence of SEQ ID NO:131. In yet another embodiment the cbsT1 gene consists of the sequence of SEQ ID NO: 131.

In one embodiment, the bile salt transporter is the bile salt transporter CbsT2. In one embodiment, the cbsT2 gene has at least about 80% identity to SEQ ID NO:132. Accordingly, in one embodiment, the cbsT2 gene has at least about 90% identity to SEQ ID NO: 132. Accordingly, in one embodiment, the cbsT2 gene has at least about 95% identity to SEQ ID NO:132. Accordingly, in one embodiment, the cbsT2 gene has at least about 70%, 75%, 80%, 85%, 86%, 87%, 88%, 89%, 90%, 91%, 92%, 93%, 94%, 95%, 96%, 97%, 98%, or 99% identity to SEQ ID NO:132. In another embodiment, the cbsT2 gene comprises the sequence of SEQ ID NO:132. In yet another embodiment the cbsT2 gene consists of the sequence of SEQ ID NO:132.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the bile salt transporter is expressed in the recombinant bacterial cells, the bacterial cells import 10% more bile salt into the bacterial cell when the transporter is expressed than unmodified bacteria of the same bacterial subtype under the same conditions. In another embodiment, when the bile salt transporter is expressed in the recombinant bacterial cells, the bacterial cells import 20%, 30%, 40%, 50%, 60%, 70%, 80%, 90% or 100% more bile salt, into the bacterial cell when the transporter is expressed than unmodified bacteria of the same bacterial subtype under the same conditions. In yet another embodiment, when the bile salt transporter is expressed in the recombinant bacterial cells, the bacterial cells import two-fold more bile salt into the cell when the transporter is expressed than unmodified bacteria of the same bacterial subtype under the same conditions. In yet another embodiment, when the bile salt transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more bile salt into the cell when the transporter is expressed than unmodified bacteria of the same bacterial subtype under the same conditions.

In one embodiment, the recombinant bacterial cells described herein comprise a first heterologous bile salt transporter and a second heterologous bile salt transporter. In one embodiment, said first bile salt transporter is derived from a different organism than said second bile salt transporter. In some embodiments, said first bile salt transporter is derived from the same organism as said second bile salt transporter. In some embodiments, said first bile salt transporter imports the same bile salt as said second bile salt transporter. In other embodiment, said first bile salt transporter imports a different bile salt from said second bile salt transporter. In some embodiments, said first bile salt transporter is a wild-type bile salt transporter and said second bile salt transporter is a mutagenized version of said first bile salt transporter. In some embodiments, the recombinant bacterial cells described herein comprise at least a third heterologous bile salt transporter. In some embodiments, the recombinant bacterial cells described herein comprise at least four heterologous bile salt transporters. In some embodiments, the recombinant bacterial cells described herein comprise at least five heterologous bile salt transporters or more.

In some embodiment, the recombinant bacterial cell comprising a heterologous gene encoding an bile salt transporter may be used to treat a disease, condition, and/or symptom associated with bile salts. In some embodiments, the recombinant bacterial cells described herein may be used to reduce, ameliorate, or eliminate one or more symptom(s) associated with these diseases or disorders. In some embodiments, the disease or disorder associated with bile salts is cardiovascular disease, metabolic disease, liver disease, such as cirrhosis or NASH, gastrointestinal cancer, and/or C. difficile infection. In some embodiments, the disclosure provides methods for reducing, ameliorating, or eliminating one or more symptom(s) associated with these diseases, including but not limited to chest pain, heart failure, or weight gain. In some embodiments, the disease is secondary to other conditions, e.g., cardiovascular disease or liver disease.

### H. Ammonia Transporters

In one embodiment, the recombinant bacterial cell of the invention comprises a heterologous gene encoding a substrate transporter, wherein the substrate transporter is an ammonia transporter. In one embodiment, the ammonia transporter transports ammonia into the cell.

The uptake of ammonia into bacterial cells is mediated by proteins well known to those of skill in the art. For example, theammonium/methylammonium transport B (AmtB) protein is a membrane transport protein that transports ammonia into bacterial cells. In one embodiment, the at least one gene encoding an ammonia transporter is an amtB gene. In one embodiment, the bacterial cell described herein has been genetically engineered to comprise at least one heterologous an amtB gene.

In some embodiments, the ammonia transporter is encoded by an ammonia transporter gene derived from a bacterial genus or species, including but not limited to, Corynebacterium, e.g., Corynebacterium glutamicum, Escherichia, e.g., Escherichia coli, Streptomyces, e.g., Streptomyces coelicolor, or Ruminococcus, e.g., Ruminococcus albus. In some embodiments, the bacterial species is Escherichia coli. In some embodiments, the bacterial species is Escherichia coli strain Nissle.

Assays for testing the activity of an ammonia transporter, a functional variant of an ammonia transporter, or a functional fragment of transporter of ammonia are well known to one of ordinary skill in the art. For example, import of ammonia may be determined using a methylammonium uptake assay, as described in Soupene et al. (1998) Proc. Natl. Acad. Sci. U.S.A. 95 (12): 7030-4, the entire contents of each of which are expressly incorporated by reference herein.

Ammonia transporters may be expressed or modified in the bacteria in order to enhance ammonia transport into the cell. Specifically, when the ammonia transporter is expressed in the recombinant bacterial cells, the bacterial cells import more ammonia into the cell when the transporter is expressed than unmodified bacteria of the same bacterial subtype under the same conditions. In one embodiment, the bacterial cell comprises a heterologous gene encoding an ammonia transporter. In one embodiment, the bacterial cell comprises a heterologous gene encoding an ammonia transporter and a genetic modification that reduces export of a ammonia, e.g., a genetic mutation in an exporter gene or promoter.

In one embodiment, the bacterial cell comprises at least one gene encoding an ammonia transporter from a different organism, e.g., a different species of bacteria. In one embodiment, the bacterial cell comprises at least one native gene encoding an ammonia transporter. In some embodiments, the at least one native gene encoding an ammonia transporter is not modified. In another embodiment, the bacterial cell comprises more than one copy of at least one native gene encoding an ammonia transporter. In yet another embodiment, the bacterial cell comprises a copy of at least one gene encoding a native ammonia transporter, as well as at least one copy of at least one heterologous gene encoding an ammonia transporter from a different bacterial species. In one embodiment, the bacterial cell comprises at least one, two, three, four, five, or six copies of the at least one heterologous gene encoding an ammonia transporter. In one embodiment, the bacterial cell comprises multiple copies of the at least one heterologous gene encoding an ammonia transporter.

In one embodiment, the recombinant bacterial cell comprises a heterologous gene encoding an ammonia transporter, wherein said ammonia transporter comprises an ammonia sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the ammonia sequence of a polypeptide encoded by an ammonia transporter gene disclosed herein.

The present disclosure further comprises genes encoding functional fragments of an ammonia transporter or functional variants of an ammonia transporter. As used herein, the term “functional fragment thereof” or “functional variant thereof” of an ammonia transporter relates to an element having qualitative biological activity in common with the wild-type ammonia transporter from which the fragment or variant was derived. For example, a functional fragment or a functional variant of a mutated ammonia transporter is one which retains essentially the same ability to import ammonia into the bacterial cell as does the ammonia transporter protein from which the functional fragment or functional variant was derived. In one embodiment, the recombinant bacterial cell comprises at least one heterologous gene encoding a functional fragment of an ammonia transporter. In another embodiment, the recombinant bacterial cell comprises a heterologous gene encoding a functional variant of an ammonia transporter.

In one embodiment, the genes encoding the ammonia transporter have been codon-optimized for use in the host organism, e.g., a bacterial cell disclosed herein. In one embodiment, the genes encoding the ammonia transporter have been codon-optimized for use in Escherichia coli.

The present disclosure also encompasses genes encoding an ammonia transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding an ammonia transporter is mutagenized; mutants exhibiting increased ammonia import are selected; and the mutagenized at least one gene encoding an ammonia transporter is isolated and inserted into the bacterial cell. In some embodiments, the at least one gene encoding an ammonia transporter is mutagenized; mutants exhibiting decreased ammonia import are selected; and the mutagenized at least one gene encoding an ammonia transporter is isolated and inserted into the bacterial cell. The transporter modifications described herein may be present on a plasmid or chromosome.

In some embodiments, the bacterial cell comprises a heterologous gene encoding an ammonia transporter operably linked to a promoter. In one embodiment, the at least one gene encoding an ammonia transporter is directly operably linked to the promoter. In another embodiment, the at least one gene encoding an ammonia transporter is indirectly operably linked to the promoter.

In one embodiment, the promoter is not operably linked with the at least one gene encoding an ammonia transporter in nature. In some embodiments, the at least one gene encoding the ammonia transporter is controlled by its native promoter. In some embodiments, the at least one gene encoding the ammonia transporter is controlled by an inducible promoter. In some embodiments, the at least one gene encoding the ammonia transporter is controlled by a promoter that is stronger than its native promoter. In some embodiments, the at least one gene encoding the ammonia transporter is controlled by a constitutive promoter.

In another embodiment, the promoter is an inducible promoter. Inducible promoters are described in more detail infra.

In one embodiment, the at least one gene encoding an ammonia transporter is located on a plasmid in the bacterial cell. In some embodiments, the plasmid is a high copy number plasmid. In some embodiments, the plasmid is a low copy number plasmid. In another embodiment, the at least one gene encoding an ammonia transporter is located in the chromosome of the bacterial cell. In yet another embodiment, a native copy of the at least one gene encoding an ammonia transporter is located in the chromosome of the bacterial cell, and a copy of at least one gene encoding an ammonia transporter from a different species of bacteria is located on a plasmid in the bacterial cell. In yet another embodiment, a native copy of the at least one gene encoding an ammonia transporter is located on a plasmid in the bacterial cell, and a copy of at least one gene encoding an ammonia transporter from a different species of bacteria is located on a plasmid in the bacterial cell. In yet another embodiment, a native copy of the at least one gene encoding an ammonia transporter is located in the chromosome of the bacterial cell, and a copy of the at least one gene encoding an ammonia transporter from a different species of bacteria is located in the chromosome of the bacterial cell.

In some embodiments, the at least one native gene encoding the ammonia transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the ammonia transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the ammonia transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the ammonia transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In one embodiment, the ammonia transporter is the ammonia transporter AmtB, for example the Escherichia coli AmtB. In one embodiment the ammonia transporter is encoded by a amtB gene. In one embodiment, the amtB gene has at least about 80% identity with the sequence of SEQ ID NO:133. Accordingly, in one embodiment, the amtB gene has at least about 90% identity with the sequence of SEQ ID NO: 133. Accordingly, in one embodiment, the amtB gene has at least about 95% identity with the sequence of SEQ ID NO: 133. Accordingly, in one embodiment, the amtB gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:133. In another embodiment, the amtB gene comprises the sequence of SEQ ID NO:133. In yet another embodiment the amtB gene consists of the sequence of SEQ ID NO:133.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the ammonia transporter is expressed in the recombinant bacterial cells, the bacterial cells import 10% more ammonia into the bacterial cell when the transporter is expressed than unmodified bacteria of the same bacterial subtype under the same conditions. In another embodiment, when the ammonia transporter is expressed in the recombinant bacterial cells, the bacterial cells import 20%, 30%, 40%, 50%, 60%, 70%, 80%, 90% or 100% more ammonia, into the bacterial cell when the transporter is expressed than unmodified bacteria of the same bacterial subtype under the same conditions. In yet another embodiment, when the ammonia transporter is expressed in the recombinant bacterial cells, the bacterial cells import two-fold more ammonia into the cell when the transporter is expressed than unmodified bacteria of the same bacterial subtype under the same conditions. In yet another embodiment, when the ammonia transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more ammonia into the cell when the transporter is expressed than unmodified bacteria of the same bacterial subtype under the same conditions.

In one embodiment, the recombinant bacterial cells described herein comprise a first heterologous ammonia transporter and a second heterologous ammonia transporter. In one embodiment, said first ammonia transporter is derived from a different organism than said second ammonia transporter. In some embodiments, said first ammonia transporter is derived from the same organism as said second ammonia transporter. In some embodiments, said first ammonia transporter is a wild-type ammonia transporter and said second ammonia transporter is a mutagenized version of said first ammonia transporter. In some embodiments, the recombinant bacterial cells described herein comprise at least a third heterologous ammonia transporter. In some embodiments, the recombinant bacterial cells described herein comprise at least four heterologous ammonia transporters. In some embodiments, the recombinant bacterial cells described herein comprise at least five heterologous ammonia transporters or more.

In some embodiment, the recombinant bacterial cell comprising a heterologous gene encoding an ammonia transporter may be used to treat a disease, condition, and/or symptom associated with hyperammonenia. In some embodiment, the recombinant bacterial cells described herein can be used to treat hepatic encephalopathy. In some embodiment, the recombinant bacterial cells described herein can be used to treat Huntington's disease. In some embodiments, the invention provides methods for reducing, ameliorating, or eliminating one or more symptom(s) associated with hepatic encephalopathy and Huntington's disease. In some embodiments, the symptom(s) associated thereof include, but are not limited to, seizures, ataxia, stroke-like lesions, coma, psychosis, vision loss, acute encephalopathy, cerebral edema, as well as vomiting, respiratory alkalosis, and hypothermia.

### I. γ-Aminobutyric Acid (GABA) Transporters

γ-aminobutyric acid (GABA) is the predominant inhibitory neurotransmitter (C₄H₉NO₂) in the mammalian central nervous system. In humans, GABA is also directly responsible for regulating muscle tone. GABA is capable of activating the GABAA receptor, which is part of a ligand-gated ion channel complex, as well as the GABAs metabotropic G protein-coupled receptor. Neurons that produce GABA are known as “GABAergic” neurons, and activation of GABA receptors is described as GABAergic tone (i.e., increased activation of GABA receptors refers to increased GABAergic tone).

Y-Aminobutyric acid (GABA) is the predominant inhibitory neurotransmitter in the mammalian central nervous system. In humans, GABA activates the postsynaptic GABAA receptor, which is part of a ligand-gated chloride-specific ion channel complex. Activation of this complex on a post-synaptic neuron allows chloride ions to enter the neuron and exert an inhibitory effect. Alterations of such GABAergic neurotransmission have been implicated in the pathophysiology of several neurological disorders, including epilepsy (Jones-Davis and MacDonald (2003) Curr. Opin. Pharmacol. 3 (1): 12-8), Huntington's disease (Krogsgaard-Larsen (1992) Pharmacol Toxicol. 70 (2): 95-104), and hepatic encephalopathy (Jones and Basile (1997) Adv. Exp. Med. Biol. 420:75-83). Neurons in the brain that are modulated by GABA are said to be under inhibitory GABAergic tone. This inhibitory tone prevents neuronal firing until a sufficiently potent stimulatory stimulus is received, or until the inhibitory tone is otherwise released. Increased GABAergic tone in hepatic encephalopathy (HE) was initially described in the early 1980s, based on a report of similar visual response patterns in rabbits with galactosamine-induced liver failure and rabbits treated with allosteric modulators of the GABAA receptor (e.g., pentobarbital, diazepam) (Jones and Basile, 1997). Clinical improvements in hepatic encephalopathy patients treated with a highly selective benzodiazapene antagonist at the GABAA receptor, flumazenil, further confirmed these observations (Banksy et al. (1985) Lancet 1:1324-5; Scollo-Lavizzari and Steinmann (1985) Lancet 1:1324. Increased GABAergic tone in HE has since been proposed as a consequence of one or more of the following: (1) increased GABA concentrations in the brain, (2) altered integrity of the GABAA receptor, and/or (3) increased concentrations of endogenous modulators of the GABAA receptor (Ahboucha and Butterworth (2004) Metab. Brain Dis. 1 9 (3-4): 331-343).

In one embodiment, the recombinant bacterial cell of the invention comprises a heterologous gene encoding a substrate transporter, wherein the substrate transporter is a GABA transporter. In one embodiment, the GABA transporter transports GABA into the cell.

The uptake of GABA into bacterial cells is mediated by proteins well known to those of skill in the art. For example, GABA uptake in E. coli is driven by membrane potential and facilitated by the membrane transport protein, GabP (Li et al. (2001) FEBS Lett. 494 (3): 165-169. GabP is a member of the amino acid/polymaine/organocation (APC) transporter superfamily, one of the two largest families of secondary active transporters (Jack et al. (2000) Microbiology 146:1797-1814). GabP protein, encoded by the gabP gene, consists of 466 amino acids and 12 transmembrane alpha helices, wherein both N- and C-termini face the cytosol (Hu and King, (1998) Biochem J. 336 (Pt 1): 69-76. The GabP residue sequence also includes a consensus amphipathic region (CAR), which is conserved between members of the APC family from bacteria to mammals (Hu and King, 1998). Upon entry into the cell, GABA is converted to succinyl semialdehyde (SSA) by GABA a-ketoglutarate transaminase (GSST). Succinate-semialdehyde dehydrogenase (SSDH) then catalyzes the second and only other specific step in GABA catabolism, the oxidation of succinyl semialdehyde to succinate (Dover and Halpern (1972) J. Bacteriol. 109 (2): 835-43). Ultimately, succinate becomes a substrate for the citric acid (TCA) cycle. In one embodiment, the at least one gene encoding a GABA transporter is encoded by an gabP gene. In one embodiment, the bacterial cell described herein has been genetically engineered to comprise at least one heterologous an gabP gene.

In some embodiments, the GABA transporter is encoded by a GABA transporter gene derived from a bacterial genus or species, including but not limited to, Bacillus, e.g., Bacillus subtilis, or Escherichia, e.g., Escherichia coli. In some embodiments, the bacterial species is Escherichia coli. In some embodiments, the bacterial species is Escherichia coli strain Nissle.

Assays for testing the activity of a GABA transporter, a functional variant of a GABA transporter, or a functional fragment of transporter of GABA are well known to one of ordinary skill in the art.

GABA transporters may be expressed or modified in the bacteria in order to enhance GABA transport into the cell. Specifically, when the GABA transporter is expressed in the recombinant bacterial cells, the bacterial cells import more GABA into the cell when the transporter is expressed than unmodified bacteria of the same bacterial subtype under the same conditions. In one embodiment, the bacterial cell comprises a heterologous gene encoding a GABA transporter. In one embodiment, the bacterial cell comprises a heterologous gene encoding a GABA transporter and a genetic modification that reduces export of a GABA, e.g., a genetic mutation in an exporter gene or promoter.

In one embodiment, the bacterial cell comprises at least one gene encoding a GABA transporter from a different organism, e.g., a different species of bacteria. In one embodiment, the bacterial cell comprises at least one native gene encoding a GABA transporter. In some embodiments, the at least one native gene encoding a GABA transporter is not modified. In another embodiment, the bacterial cell comprises more than one copy of at least one native gene encoding a GABA transporter. In yet another embodiment, the bacterial cell comprises a copy of at least one gene encoding a native GABA transporter, as well as at least one copy of at least one heterologous gene encoding a GABA transporter from a different bacterial species. In one embodiment, the bacterial cell comprises at least one, two, three, four, five, or six copies of the at least one heterologous gene encoding a GABA transporter. In one embodiment, the bacterial cell comprises multiple copies of the at least one heterologous gene encoding a GABA transporter.

In one embodiment, the recombinant bacterial cell comprises a heterologous gene encoding a GABA transporter, wherein said GABA transporter comprises a GABA sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the GABA sequence of a polypeptide encoded by a GABA transporter gene disclosed herein.

The present disclosure further comprises genes encoding functional fragments of a GABA transporter or functional variants of a GABA transporter. As used herein, the term “functional fragment thereof” or “functional variant thereof” of a GABA transporter relates to an element having qualitative biological activity in common with the wild-type GABA transporter from which the fragment or variant was derived. For example, a functional fragment or a functional variant of a mutated GABA transporter is one which retains essentially the same ability to import GABA into the bacterial cell as does the GABA transporter protein from which the functional fragment or functional variant was derived. In one embodiment, the recombinant bacterial cell comprises at least one heterologous gene encoding a functional fragment of a GABA transporter. In another embodiment, the recombinant bacterial cell comprises a heterologous gene encoding a functional variant of a GABA transporter.

In one embodiment, the genes encoding the GABA transporter have been codon-optimized for use in the host organism, e.g., a bacterial cell disclosed herein. In one embodiment, the genes encoding the GABA transporter have been codon-optimized for use in Escherichia coli.

The present disclosure also encompasses genes encoding a GABA transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a GABA transporter is mutagenized; mutants exhibiting increased GABA import are selected; and the mutagenized at least one gene encoding a GABA transporter is isolated and inserted into the bacterial cell. In some embodiments, the at least one gene encoding a GABA transporter is mutagenized; mutants exhibiting decreased GABA import are selected; and the mutagenized at least one gene encoding a GABA transporter is isolated and inserted into the bacterial cell. The transporter modifications described herein may be present on a plasmid or chromosome.

In some embodiments, the bacterial cell comprises a heterologous gene encoding a GABA transporter operably linked to a promoter. In one embodiment, the at least one gene encoding a GABA transporter is directly operably linked to the promoter. In another embodiment, the at least one gene encoding a GABA transporter is indirectly operably linked to the promoter.

In one embodiment, the promoter is not operably linked with the at least one gene encoding a GABA transporter in nature. In some embodiments, the at least one gene encoding the GABA transporter is controlled by its native promoter. In some embodiments, the at least one gene encoding the GABA transporter is controlled by an inducible promoter. In some embodiments, the at least one gene encoding the GABA transporter is controlled by a promoter that is stronger than its native promoter. In some embodiments, the at least one gene encoding the GABA transporter is controlled by a constitutive promoter.

In another embodiment, the promoter is an inducible promoter. Inducible promoters are described in more detail infra.

In one embodiment, the at least one gene encoding a GABA transporter is located on a plasmid in the bacterial cell. In some embodiments, the plasmid is a high copy number plasmid. In some embodiments, the plasmid is a low copy number plasmid. In another embodiment, the at least one gene encoding a GABA transporter is located in the chromosome of the bacterial cell. In yet another embodiment, a native copy of the at least one gene encoding a GABA transporter is located in the chromosome of the bacterial cell, and a copy of at least one gene encoding a GABA transporter from a different species of bacteria is located on a plasmid in the bacterial cell. In yet another embodiment, a native copy of the at least one gene encoding a GABA transporter is located on a plasmid in the bacterial cell, and a copy of at least one gene encoding a GABA transporter from a different species of bacteria is located on a plasmid in the bacterial cell. In yet another embodiment, a native copy of the at least one gene encoding a GABA transporter is located in the chromosome of the bacterial cell, and a copy of the at least one gene encoding a GABA transporter from a different species of bacteria is located in the chromosome of the bacterial cell.

In some embodiments, the at least one native gene encoding the GABA transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the GABA transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the GABA transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the GABA transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In one embodiment, the GABA transporter is the GABA transporter GabP, for example the Escherichia coli GabP. In one embodiment the GABA transporter is encoded by a amtB gene. In one embodiment, the gabP gene has at least about 80% identity with the sequence of SEQ ID NO:134. Accordingly, in one embodiment, the gabP gene has at least about 90% identity with the sequence of SEQ ID NO: 134. Accordingly, in one embodiment, the gabP gene has at least about 95% identity with the sequence of SEQ ID NO:134. Accordingly, in one embodiment, the gabP gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:134. In another embodiment, the gabP gene comprises the sequence of SEQ ID NO:134. In yet another embodiment the gabP gene consists of the sequence of SEQ ID NO:134.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the GABA transporter is expressed in the recombinant bacterial cells, the bacterial cells import 10% more GABA into the bacterial cell when the transporter is expressed than unmodified bacteria of the same bacterial subtype under the same conditions. In another embodiment, when the GABA transporter is expressed in the recombinant bacterial cells, the bacterial cells import 20%, 30%, 40%, 50%, 60%, 70%, 80%, 90% or 100% more GABA, into the bacterial cell when the transporter is expressed than unmodified bacteria of the same bacterial subtype under the same conditions. In yet another embodiment, when the GABA transporter is expressed in the recombinant bacterial cells, the bacterial cells import two-fold more GABA into the cell when the transporter is expressed than unmodified bacteria of the same bacterial subtype under the same conditions. In yet another embodiment, when the GABA transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more GABA into the cell when the transporter is expressed than unmodified bacteria of the same bacterial subtype under the same conditions.

In one embodiment, the recombinant bacterial cells described herein comprise a first heterologous GABA transporter and a second heterologous GABA transporter. In one embodiment, said first GABA transporter is derived from a different organism than said second GABA transporter. In some embodiments, said first GABA transporter is derived from the same organism as said second GABA transporter. In some embodiments, said first GABA transporter is a wild-type GABA transporter and said second GABA transporter is a mutagenized version of said first GABA transporter. In some embodiments, the recombinant bacterial cells described herein comprise at least a third heterologous GABA transporter. In some embodiments, the recombinant bacterial cells described herein comprise at least four heterologous GABA transporters. In some embodiments, the recombinant bacterial cells described herein comprise at least five heterologous GABA transporters or more.

In some embodiment, the recombinant bacterial cell comprising a heterologous gene encoding an GABA transporter may be used to treat a disease, condition, and/or symptom associated with hyperammonenia. In some embodiment, the recombinant bacterial cells described herein can be used to treat hepatic encephalopathy. In some embodiment, the recombinant bacterial cells described herein can be used to treat Huntington's disease. In some embodiments, the invention provides methods for reducing, ameliorating, or eliminating one or more symptom(s) associated with hepatic encephalopathy and Huntington's disease. In some embodiments, the symptom(s) associated thereof include, but are not limited to, seizures, ataxia, stroke-like lesions, coma, psychosis, vision loss, acute encephalopathy, cerebral edema, as well as vomiting, respiratory alkalosis, and hypothermia.

### J. Manganese Transporters

In biological systems, manganese (Mn²⁺) is an essential trace metal and plays an important role in enzyme-mediated catalysis, but can also have deleterious effects. Manganese is a biologically important trace metal and is required for the survival of most living organisms. Cells maintain manganese under tight homeostatic control in order to avoid toxicity. In mammals, manganese is excreted in the bile, but its disposal is affected by the impaired flow of bile from the liver to the duodenum (i.e., cholestasis) that accompanies liver failure. Similar to ammonia, elevated concentrations of manganese play a role in the development of hepatic encephalopathy (Rivera-Manda et al. (2012) Neurochem. Res. 37 (5): 1074-1084). Astrocytes in the brain which detoxify ammonia in a reaction catalyzed by glutamine synthetase, require manganese as a cofactor and thus have a tendency to accumulate this metal (Aschner et al. (1999) Neurotoxicology 20 (2-3): 173-180) . . . . In vitro studies have demonstrated that manganese can result in the inhibition of glutamate transport (Hazell and Norenberg, 1997), abnormalities in astrocyte morphology (Hazell et al. (2006) Neurosci. Lett. 396 (3): 167-71), and increased cell volume (Rama Rao et al., 2007). Some disorders associated with hyperammonemia may also be characterized by elevated levels of manganese; manganese may contribute to disease pathogenesis (e.g., hepatic encephalopathy) (Rivera-Manda et al., 2012). Manganese and ammonia have also been shown to act synergistically in the pathogenesis of hepatic encephalopathy (Jayakumar et al. (2004) Neurochem. Res. 29 (11): 2051-6).

In one embodiment, the recombinant bacterial cell of the invention comprises a heterologous gene encoding a substrate transporter, wherein the substrate transporter is a manganese transporter. In one embodiment, the manganese transporter transports manganese into the cell.

The uptake of manganese into bacterial cells is mediated by proteins well known to those of skill in the art. For example, the manganese transporter MntH is a membrane transport protein capable of transporting manganese into bacterial cells (see, e.g., Jensen and Jensen (2014) Chapter 1: Manganese transport, trafficking and function in invertebrates. In: Manganese in Health and Disease, pp. 1-33). In Escherichia coli, the mntH gene encodes a proton-stimulated, divalent metal cation uptake system involved in manganese transport (Porcheron et al. (2013) Front. Cell. Infect. Microbiol. 3:90). In one embodiment, the manganese transporter is selected from the group consisting of mntH, MntABCD, SitABCD, PsaABCD, YfcABCD. In one embodiment, the at least one gene encoding a manganese transporter is encoded by an mntH gene. In one embodiment, the at least one gene encoding a manganese transporter is encoded by an MntABCD operon. In one embodiment, the at least one gene encoding a manganese transporter is encoded by an sitABCD operon. In one embodiment, the at least one gene encoding a manganese transporter is encoded by an PsaABCD operon. In one embodiment, the at least one gene encoding a manganese transporter is encoded by an YfeABCD operon. In one embodiment, the bacterial cell described herein has been genetically engineered to comprise at least one heterologous mntH gene.

Metal ion homeostasis in prokaryotic cells, which lack internal compartmentalization, is maintained by the tight regulation of metal ion flux across in cytoplasmic membrane (Jensen and Jensen, 2014). Manganese uptake in bacteria predominantly involves two major types of transporters: proton-dependent Nramprelated transporters, and/or ATP-dependent ABC transporters. The Nramp (Natural resistance-§.ssociated macrophage Qrotein) transporter family was first described in plants, animals, and yeasts (Cellier et al. (1996) Trends Genet. 12 (6): 201-4), but MntH has since been characterized in several bacterial species (Porcheron et al., 2013). Selectivity of the Nramp1 transporter for manganese has been shown in metal accumulation studies, wherein overexpression of Staphylococcus aureus mntH resulted in increased levels of cell-associated manganese, but no accumulation of calcium, copper, iron, magnesium, or zinc (Horsburgh et al. (2002) Mol. Microbiol. 44 (5): 1269-86). Additionally, Bacillus subtilis strains comprising a mutation in the mntH gene exhibited impaired growth in metal-free medium that was rescued by the addition of manganese (Que and Heimann (2000) Mol. Microbiol. 35 (6): 1454-68).

High-affinity manganese uptake may also be mediated by ABC (ATP-binding cassette) transporters. Members of this transporter superfamily utilize the hydrolysis of ATP to fuel the import or export of diverse substrates, ranging from ions to macromolecules, and are well characterized for their role in multi-drug resistance in both prokaryotic and eukaryotic cells. Non-limiting examples of bacterial ABC transporters involved in manganese import include MntABCD (Bacillus subtilis, Staphylococcus aureus), SitABCD (Salmonella typhimurium, Shigella flexneri), PsaABCD (Streptococcus pneumoniae), and YfeABCD (Yersinia pestis) (Bearden and Perry (1999) Mol. Microbiol. 32 (2): 403-14; Kehres et al. (2002) J. Bacteriol. 184 (12): 3159-66; McAllister et al. (2004) Mol. Microbiol. 53 (3): 889-901; Zhou et al. (1999) Infect. Immun. 67 (4): 1974-81). The MntABCD transporter complex consists of three subunits, wherein MntC and MntD are integral membrane proteins that comprise the permease subunit mediate cation transport, MntB is the ATPase, and MntA binds and delivers manganese to the permease submit. Other ABC transporter operons, such as sitABCD, psaABCD, and yfeABCD, exhibit similar subunit organization and function (Higgins, 1992; Rccs et al. (2009) Nat. Rev. Mol. Cell Biol. 10 (3): 218-227).

In some embodiments, the manganese transporter is encoded by a manganese transporter gene derived from a bacterial genus or species, including but not limited to, Bacillus, e.g., Bacillus subtilis, Staphylococcus, e.g., Staphylococcus aureus, Salmonella, e.g., Salmonella typhimurium, Shigella, e.g., Shigella flexneri, Yersinia, e.g., Yersinia pestis, or Escherichia, e.g., Escherichia coli. In some embodiments, the bacterial species is Escherichia coli. In some embodiments, the bacterial species is Escherichia coli strain Nissle.

Assays for testing the activity of a manganese transporter, a functional variant of a manganese transporter, or a functional fragment of transporter of manganese are well known to one of ordinary skill in the art.

Manganese transporters may be expressed or modified in the bacteria in order to enhance manganese transport into the cell. Specifically, when the manganese transporter is expressed in the recombinant bacterial cells, the bacterial cells import more manganese into the cell when the transporter is expressed than unmodified bacteria of the same bacterial subtype under the same conditions. In one embodiment, the bacterial cell comprises a heterologous gene encoding a manganese transporter. In one embodiment, the bacterial cell comprises a heterologous gene encoding a manganese transporter and a genetic modification that reduces export of a manganese, e.g., a genetic mutation in an exporter gene or promoter.

In one embodiment, the bacterial cell comprises at least one gene encoding a manganese transporter from a different organism, e.g., a different species of bacteria. In one embodiment, the bacterial cell comprises at least one native gene encoding a manganese transporter. In some embodiments, the at least one native gene encoding a manganese transporter is not modified. In another embodiment, the bacterial cell comprises more than one copy of at least one native gene encoding a manganese transporter. In yet another embodiment, the bacterial cell comprises a copy of at least one gene encoding a native manganese transporter, as well as at least one copy of at least one heterologous gene encoding a manganese transporter from a different bacterial species. In one embodiment, the bacterial cell comprises at least one, two, three, four, five, or six copies of the at least one heterologous gene encoding a manganese transporter. In one embodiment, the bacterial cell comprises multiple copies of the at least one heterologous gene encoding a manganese transporter.

In one embodiment, the recombinant bacterial cell comprises a heterologous gene encoding a manganese transporter, wherein said manganese transporter comprises a manganese sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the manganese sequence of a polypeptide encoded by a manganese transporter gene disclosed herein.

The present disclosure further comprises genes encoding functional fragments of a manganese transporter or functional variants of a manganese transporter. As used herein, the term “functional fragment thereof” or “functional variant thereof” of a manganese transporter relates to an element having qualitative biological activity in common with the wild-type manganese transporter from which the fragment or variant was derived. For example, a functional fragment or a functional variant of a mutated manganese transporter is one which retains essentially the same ability to import manganese into the bacterial cell as does the manganese transporter protein from which the functional fragment or functional variant was derived. In one embodiment, the recombinant bacterial cell comprises at least one heterologous gene encoding a functional fragment of a manganese transporter. In another embodiment, the recombinant bacterial cell comprises a heterologous gene encoding a functional variant of a manganese transporter.

In one embodiment, the genes encoding the manganese transporter have been codon-optimized for use in the host organism, e.g., a bacterial cell disclosed herein. In one embodiment, the genes encoding the manganese transporter have been codon-optimized for use in Escherichia coli.

The present disclosure also encompasses genes encoding a manganese transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a manganese transporter is mutagenized; mutants exhibiting increased manganese import are selected; and the mutagenized at least one gene encoding a manganese transporter is isolated and inserted into the bacterial cell. In some embodiments, the at least one gene encoding a manganese transporter is mutagenized; mutants exhibiting decreased manganese import are selected; and the mutagenized at least one gene encoding a manganese transporter is isolated and inserted into the bacterial cell. The transporter modifications described herein may be present on a plasmid or chromosome.

In some embodiments, the bacterial cell comprises a heterologous gene encoding a manganese transporter operably linked to a promoter. In one embodiment, the at least one gene encoding a manganese transporter is directly operably linked to the promoter. In another embodiment, the at least one gene encoding a manganese transporter is indirectly operably linked to the promoter.

In one embodiment, the promoter is not operably linked with the at least one gene encoding a manganese transporter in nature. In some embodiments, the at least one gene encoding the manganese transporter is controlled by its native promoter. In some embodiments, the at least one gene encoding the manganese transporter is controlled by an inducible promoter. In some embodiments, the at least one gene encoding the manganese transporter is controlled by a promoter that is stronger than its native promoter. In some embodiments, the at least one gene encoding the manganese transporter is controlled by a constitutive promoter.

In another embodiment, the promoter is an inducible promoter. Inducible promoters are described in more detail infra.

In one embodiment, the at least one gene encoding a manganese transporter is located on a plasmid in the bacterial cell. In some embodiments, the plasmid is a high copy number plasmid. In some embodiments, the plasmid is a low copy number plasmid. In another embodiment, the at least one gene encoding a manganese transporter is located in the chromosome of the bacterial cell. In yet another embodiment, a native copy of the at least one gene encoding a manganese transporter is located in the chromosome of the bacterial cell, and a copy of at least one gene encoding a manganese transporter from a different species of bacteria is located on a plasmid in the bacterial cell. In yet another embodiment, a native copy of the at least one gene encoding a manganese transporter is located on a plasmid in the bacterial cell, and a copy of at least one gene encoding a manganese transporter from a different species of bacteria is located on a plasmid in the bacterial cell. In yet another embodiment, a native copy of the at least one gene encoding a manganese transporter is located in the chromosome of the bacterial cell, and a copy of the at least one gene encoding a manganese transporter from a different species of bacteria is located in the chromosome of the bacterial cell.

In some embodiments, the at least one native gene encoding the manganese transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the manganese transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the manganese transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the manganese transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In one embodiment, the manganese transporter is the manganese transporter GabP, for example the Escherichia coli mntH gene. In one embodiment the manganese transporter is encoded by a mntH gene. In one embodiment, the mntH gene has at least about 80% identity with the sequence of SEQ ID NO: 135. Accordingly, in one embodiment, the mntH gene has at least about 90% identity with the sequence of SEQ ID NO: 135. Accordingly, in one embodiment, the mntH gene has at least about 95% identity with the sequence of SEQ ID NO: 135. Accordingly, in one embodiment, the mntH gene has at least about 70%, 75%, 80%, 85%, 86%, 87%, 88%, 89%, 90%, 91%, 92%, 93%, 94%, 95%, 96%, 97%, 98%, or 99% identity with the sequence of SEQ ID NO:135. In another embodiment, the mntH gene comprises the sequence of SEQ ID NO: 135. In yet another embodiment the mntH gene consists of the sequence of SEQ ID NO:135.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the manganese transporter is expressed in the recombinant bacterial cells, the bacterial cells import 10% more manganese into the bacterial cell when the transporter is expressed than unmodified bacteria of the same bacterial subtype under the same conditions. In another embodiment, when the manganese transporter is expressed in the recombinant bacterial cells, the bacterial cells import 20%, 30%, 40%, 50%, 60%, 70%, 80%, 90% or 100% more manganese, into the bacterial cell when the transporter is expressed than unmodified bacteria of the same bacterial subtype under the same conditions. In yet another embodiment, when the manganese transporter is expressed in the recombinant bacterial cells, the bacterial cells import two-fold more manganese into the cell when the transporter is expressed than unmodified bacteria of the same bacterial subtype under the same conditions. In yet another embodiment, when the manganese transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more manganese into the cell when the transporter is expressed than unmodified bacteria of the same bacterial subtype under the same conditions.

In one embodiment, the recombinant bacterial cells described herein comprise a first heterologous manganese transporter and a second heterologous manganese transporter. In one embodiment, said first manganese transporter is derived from a different organism than said second manganese transporter. In some embodiments, said first manganese transporter is derived from the same organism as said second manganese transporter. In some embodiments, said first manganese transporter is a wild-type manganese transporter and said second manganese transporter is a mutagenized version of said first manganese transporter. In some embodiments, the recombinant bacterial cells described herein comprise at least a third heterologous manganese transporter. In some embodiments, the recombinant bacterial cells described herein comprise at least four heterologous manganese transporters. In some embodiments, the recombinant bacterial cells described herein comprise at least five heterologous manganese transporters or more.

In some embodiment, the recombinant bacterial cell comprising a heterologous gene encoding an manganese transporter may be used to treat a disease, condition, and/or symptom associated with hyperammonenia. In some embodiment, the recombinant bacterial cells described herein can be used to treat hepatic encephalopathy. In some embodiment, the recombinant bacterial cells described herein can be used to treat Huntington's disease. In some embodiments, the invention provides methods for reducing, ameliorating, or eliminating one or more symptom(s) associated with hepatic encephalopathy and Huntington's disease. In some embodiments, the symptom(s) associated thereof include, but are not limited to, seizures, ataxia, stroke-like lesions, coma, psychosis, vision loss, acute encephalopathy, cerebral edema, as well as vomiting, respiratory alkalosis, and hypothermia.

### K. Toxin Transporters

In one embodiment, the recombinant bacterial cell of the invention comprises a heterologous gene encoding a substrate transporter, wherein the substrate transporter is a toxin transporter. In one embodiment, the toxin transporter transports toxin into the cell.

In some embodiments, the toxin transporter is encoded by a toxin transporter gene derived from a bacterial genus or species, including but not limited to, Escherichia, Corynebacterium, Escherichia coli, Saccharomyces cerevisiae or Corynebacterium glutamicum. In some embodiments, the bacterial species is Escherichia coli. In some embodiments, the bacterial species is Escherichia coli strain Nissle.

Assays for testing the activity of a toxin transporter, a functional variant of a toxin transporter, or a functional fragment of transporter of toxin are well known to one of ordinary skill in the art.

Toxin transporters may be expressed or modified in the bacteria in order to enhance toxin transport into the cell. Specifically, when the toxin transporter is expressed in the recombinant bacterial cells, the bacterial cells import more toxin into the cell when the transporter is expressed than unmodified bacteria of the same bacterial subtype under the same conditions. In one embodiment, the bacterial cell comprises a heterologous gene encoding a toxin transporter. In one embodiment, the bacterial cell comprises a heterologous gene encoding a toxin transporter and a genetic modification that reduces export of a toxin, e.g., a genetic mutation in an exporter gene or promoter.

In one embodiment, the bacterial cell comprises at least one gene encoding a toxin transporter from a different organism, e.g., a different species of bacteria. In one embodiment, the bacterial cell comprises at least one native gene encoding a toxin transporter. In some embodiments, the at least one native gene encoding a toxin transporter is not modified. In another embodiment, the bacterial cell comprises more than one copy of at least one native gene encoding a toxin transporter. In yet another embodiment, the bacterial cell comprises a copy of at least one gene encoding a native toxin transporter, as well as at least one copy of at least one heterologous gene encoding a toxin transporter from a different bacterial species. In one embodiment, the bacterial cell comprises at least one, two, three, four, five, or six copies of the at least one heterologous gene encoding a toxin transporter. In one embodiment, the bacterial cell comprises multiple copies of the at least one heterologous gene encoding a toxin transporter.

In one embodiment, the recombinant bacterial cell comprises a heterologous gene encoding a toxin transporter, wherein said toxin transporter comprises a toxin sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the toxin sequence of a polypeptide encoded by a toxin transporter gene disclosed herein.

The present disclosure further comprises genes encoding functional fragments of a toxin transporter or functional variants of a toxin transporter. As used herein, the term “functional fragment thereof” or “functional variant thereof” of a toxin transporter relates to an element having qualitative biological activity in common with the wild-type toxin transporter from which the fragment or variant was derived. For example, a functional fragment or a functional variant of a mutated toxin transporter is one which retains essentially the same ability to import toxin into the bacterial cell as does the toxin transporter protein from which the functional fragment or functional variant was derived. In one embodiment, the recombinant bacterial cell comprises at least one heterologous gene encoding a functional fragment of a toxin transporter. In another embodiment, the recombinant bacterial cell comprises a heterologous gene encoding a functional variant of a toxin transporter.

In one embodiment, the genes encoding the toxin transporter have been codon-optimized for use in the host organism, e.g., a bacterial cell disclosed herein. In one embodiment, the genes encoding the toxin transporter have been codon-optimized for use in Escherichia coli.

The present disclosure also encompasses genes encoding a toxin transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a toxin transporter is mutagenized; mutants exhibiting increased toxin import are selected; and the mutagenized at least one gene encoding a toxin transporter is isolated and inserted into the bacterial cell. In some embodiments, the at least one gene encoding a toxin transporter is mutagenized; mutants exhibiting decreased toxin import are selected; and the mutagenized at least one gene encoding a toxin transporter is isolated and inserted into the bacterial cell. The transporter modifications described herein may be present on a plasmid or chromosome.

In some embodiments, the bacterial cell comprises a heterologous gene encoding a toxin transporter operably linked to a promoter. In one embodiment, the at least one gene encoding a toxin transporter is directly operably linked to the promoter. In another embodiment, the at least one gene encoding a toxin transporter is indirectly operably linked to the promoter.

In one embodiment, the promoter is not operably linked with the at least one gene encoding a toxin transporter in nature. In some embodiments, the at least one gene encoding the toxin transporter is controlled by its native promoter. In some embodiments, the at least one gene encoding the toxin transporter is controlled by an inducible promoter. In some embodiments, the at least one gene encoding the toxin transporter is controlled by a promoter that is stronger than its native promoter. In some embodiments, the at least one gene encoding the toxin transporter is controlled by a constitutive promoter.

In another embodiment, the promoter is an inducible promoter. Inducible promoters are described in more detail infra.

In one embodiment, the at least one gene encoding a toxin transporter is located on a plasmid in the bacterial cell. In some embodiments, the plasmid is a high copy number plasmid. In some embodiments, the plasmid is a low copy number plasmid. In another embodiment, the at least one gene encoding a toxin transporter is located in the chromosome of the bacterial cell. In yet another embodiment, a native copy of the at least one gene encoding a toxin transporter is located in the chromosome of the bacterial cell, and a copy of at least one gene encoding a toxin transporter from a different species of bacteria is located on a plasmid in the bacterial cell. In yet another embodiment, a native copy of the at least one gene encoding a toxin transporter is located on a plasmid in the bacterial cell, and a copy of at least one gene encoding a toxin transporter from a different species of bacteria is located on a plasmid in the bacterial cell. In yet another embodiment, a native copy of the at least one gene encoding a toxin transporter is located in the chromosome of the bacterial cell, and a copy of the at least one gene encoding a toxin transporter from a different species of bacteria is located in the chromosome of the bacterial cell.

In some embodiments, the at least one native gene encoding the toxin transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the toxin transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the toxin transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the toxin transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the toxin transporter is expressed in the recombinant bacterial cells, the bacterial cells import 10% more toxin into the bacterial cell when the transporter is expressed than unmodified bacteria of the same bacterial subtype under the same conditions. In another embodiment, when the toxin transporter is expressed in the recombinant bacterial cells, the bacterial cells import 20%, 30%, 40%, 50%, 60%, 70%, 80%, 90% or 100% more PGE2, into the bacterial cell when the transporter is expressed than unmodified bacteria of the same bacterial subtype under the same conditions. In yet another embodiment, when the toxin transporter is expressed in the recombinant bacterial cells, the bacterial cells import two-fold more toxin into the cell when the transporter is expressed than unmodified bacteria of the same bacterial subtype under the same conditions. In yet another embodiment, when the toxin transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more toxin into the cell when the transporter is expressed than unmodified bacteria of the same bacterial subtype under the same conditions.

In one embodiment, the recombinant bacterial cells described herein comprise a first heterologous toxin transporter and a second heterologous toxin transporter. In one embodiment, said first toxin transporter is derived from a different organism than said second toxin transporter. In some embodiments, said first toxin transporter is derived from the same organism as said second toxin transporter. In some embodiments, said first toxin transporter imports the same toxin as said second toxin transporter. In other embodiment, said first toxin transporter imports a different toxin from said second toxin transporter. In some embodiments, said first toxin transporter is a wild-type toxin transporter and said second toxin transporter is a mutagenized version of said first toxin transporter. In some embodiments, the recombinant bacterial cells described herein comprise at least a third heterologous toxin transporter. In some embodiments, the recombinant bacterial cells described herein comprise at least four heterologous toxin transporters. In some embodiments, the recombinant bacterial cells described herein comprise at least five heterologous toxin transporters or more.

### L. Peptide Transporters

In one embodiment, the recombinant bacterial cell of the invention comprises a heterologous gene encoding a substrate transporter, wherein the substrate transporter is a peptide transporter.

In some embodiments, the peptide transporter is encoded by a peptide transporter gene derived from a bacterial genus or species, including but not limited to, Escherichia, Corynebacterium, Escherichia coli, Saccharomyces cerevisiae or Corynebacterium glutamicum. In some embodiments, the bacterial species is Escherichia coli. In some embodiments, the bacterial species is Escherichia coli strain Nissle.

Assays for testing the activity of a peptide transporter, a functional variant of a peptide transporter, or a functional fragment of transporter of peptide are well known to one of ordinary skill in the art.

Peptide transporters may be expressed or modified in the bacteria in order to enhance peptide transport into the cell. Specifically, when the peptide transporter is expressed in the recombinant bacterial cells, the bacterial cells import more peptide into the cell when the transporter is expressed than unmodified bacteria of the same bacterial subtype under the same conditions. In one embodiment, the bacterial cell comprises a heterologous gene encoding a peptide transporter. In one embodiment, the bacterial cell comprises a heterologous gene encoding a peptide transporter and a genetic modification that reduces export of a peptide, e.g., a genetic mutation in an exporter gene or promoter.

In one embodiment, the bacterial cell comprises at least one gene encoding a peptide transporter from a different organism, e.g., a different species of bacteria. In one embodiment, the bacterial cell comprises at least one native gene encoding a peptide transporter. In some embodiments, the at least one native gene encoding a peptide transporter is not modified. In another embodiment, the bacterial cell comprises more than one copy of at least one native gene encoding a peptide transporter. In yet another embodiment, the bacterial cell comprises a copy of at least one gene encoding a native peptide transporter, as well as at least one copy of at least one heterologous gene encoding a peptide transporter from a different bacterial species. In one embodiment, the bacterial cell comprises at least one, two, three, four, five, or six copies of the at least one heterologous gene encoding a peptide transporter. In one embodiment, the bacterial cell comprises multiple copies of the at least one heterologous gene encoding a peptide transporter.

In one embodiment, the recombinant bacterial cell comprises a heterologous gene encoding a peptide transporter, wherein said peptide transporter comprises a peptide sequence that has at least 70%, 75%, 80%, 81%, 82%, 83% 84%, 85%, 86%, 87%, 88%, 89%, 90%, 91%, 92%, 93%, 94%, 95%, 96%, 97%, 98%, 99%, or 100% identity to the peptide sequence of a polypeptide encoded by a peptide transporter gene disclosed herein.

The present disclosure further comprises genes encoding functional fragments of a peptide transporter or functional variants of a peptide transporter. As used herein, the term “functional fragment thereof” or “functional variant thereof” of a peptide transporter relates to an element having qualitative biological activity in common with the wild-type peptide transporter from which the fragment or variant was derived. For example, a functional fragment or a functional variant of a mutated peptide transporter is one which retains essentially the same ability to import peptide into the bacterial cell as does the peptide transporter protein from which the functional fragment or functional variant was derived. In one embodiment, the recombinant bacterial cell comprises at least one heterologous gene encoding a functional fragment of a peptide transporter. In another embodiment, the recombinant bacterial cell comprises a heterologous gene encoding a functional variant of a peptide transporter.

In one embodiment, the genes encoding the peptide transporter have been codon-optimized for use in the host organism, e.g., a bacterial cell disclosed herein. In one embodiment, the genes encoding the peptide transporter have been codon-optimized for use in Escherichia coli.

The present disclosure also encompasses genes encoding a peptide transporter comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding a peptide transporter is mutagenized; mutants exhibiting increased peptide import are selected; and the mutagenized at least one gene encoding a peptide transporter is isolated and inserted into the bacterial cell. In some embodiments, the at least one gene encoding a peptide transporter is mutagenized; mutants exhibiting decreased peptide import are selected; and the mutagenized at least one gene encoding a peptide transporter is isolated and inserted into the bacterial cell. The transporter modifications described herein may be present on a plasmid or chromosome.

In some embodiments, the bacterial cell comprises a heterologous gene encoding a peptide transporter operably linked to a promoter. In one embodiment, the at least one gene encoding a peptide transporter is directly operably linked to the promoter. In another embodiment, the at least one gene encoding a peptide transporter is indirectly operably linked to the promoter.

In one embodiment, the promoter is not operably linked with the at least one gene encoding a peptide transporter in nature. In some embodiments, the at least one gene encoding the peptide transporter is controlled by its native promoter. In some embodiments, the at least one gene encoding the peptide transporter is controlled by an inducible promoter. In some embodiments, the at least one gene encoding the peptide transporter is controlled by a promoter that is stronger than its native promoter. In some embodiments, the at least one gene encoding the peptide transporter is controlled by a constitutive promoter.

In another embodiment, the promoter is an inducible promoter. Inducible promoters are described in more detail infra.

In one embodiment, the at least one gene encoding a peptide transporter is located on a plasmid in the bacterial cell. In some embodiments, the plasmid is a high copy number plasmid. In some embodiments, the plasmid is a low copy number plasmid. In another embodiment, the at least one gene encoding a peptide transporter is located in the chromosome of the bacterial cell. In yet another embodiment, a native copy of the at least one gene encoding a peptide transporter is located in the chromosome of the bacterial cell, and a copy of at least one gene encoding a peptide transporter from a different species of bacteria is located on a plasmid in the bacterial cell. In yet another embodiment, a native copy of the at least one gene encoding a peptide transporter is located on a plasmid in the bacterial cell, and a copy of at least one gene encoding a peptide transporter from a different species of bacteria is located on a plasmid in the bacterial cell. In yet another embodiment, a native copy of the at least one gene encoding a peptide transporter is located in the chromosome of the bacterial cell, and a copy of the at least one gene encoding a peptide transporter from a different species of bacteria is located in the chromosome of the bacterial cell.

In some embodiments, the at least one native gene encoding the peptide transporter in the recombinant bacterial cell is not modified, and one or more additional copies of the native transporter are inserted into the genome. In alternate embodiments, the at least one native gene encoding the transporter is not modified, and one or more additional copies of the transporter from a different bacterial species is inserted into the genome of the recombinant bacterial cell. In some embodiments, the at least one native gene encoding the peptide transporter in the recombinant bacterial cell is modified, and one or more additional copies of the native transporter are inserted into the genome. In alternate embodiments, the at least one native gene encoding the transporter is modified, and one or more additional copies of the transporter from a different bacterial species is inserted into the genome of the recombinant bacterial cell.

In some embodiments, at least one native gene encoding the peptide transporter in the bacterial cell is not modified, and one or more additional copies of at least one native gene encoding the transporter are present in the bacterial cell on a plasmid. In alternate embodiments, the at least one native gene encoding the transporter is not modified, and a copy of at least one gene encoding the transporter from a different bacterial species is present in the bacteria on a plasmid. In some embodiments, at least one native gene encoding the peptide transporter in the bacterial cell is modified, and one or more additional copies of at least one native gene encoding the transporter are present in the bacterial cell on a plasmid. In alternate embodiments, the at least one native gene encoding the transporter is modified, and a copy of at least one gene encoding the transporter from a different bacterial species is present in the bacteria on a plasmid.

In some embodiments, the bacterium is E. coli Nissle, and the at least one native gene encoding the transporter in E. coli Nissle is not modified; one or more additional copies at least one native gene encoding the transporter from E. coli Nissle is inserted into the E. coli Nissle genome. In an alternate embodiment, the at least one native gene encoding the transporter in E. coli Nissle is not modified, and a copy of at least one gene encoding the transporter from a different bacterial species is inserted into the E. coli Nissle genome.

In one embodiment, when the peptide transporter is expressed in the recombinant bacterial cells, the bacterial cells import 10% more peptide into the bacterial cell when the transporter is expressed than unmodified bacteria of the same bacterial subtype under the same conditions. In another embodiment, when the peptide transporter is expressed in the recombinant bacterial cells, the bacterial cells import 20%, 30%, 40%, 50%, 60%, 70%, 80%, 90% or 100% more PGE2, into the bacterial cell when the transporter is expressed than unmodified bacteria of the same bacterial subtype under the same conditions. In yet another embodiment, when the peptide transporter is expressed in the recombinant bacterial cells, the bacterial cells import two-fold more peptide into the cell when the transporter is expressed than unmodified bacteria of the same bacterial subtype under the same conditions. In yet another embodiment, when the peptide transporter is expressed in the recombinant bacterial cells, the bacterial cells import three-fold, four-fold, five-fold, six-fold, seven-fold, eight-fold, nine-fold, ten-fold, fifteen-fold, twenty-fold, thirty-fold, forty-fold, or fifty-fold more peptide into the cell when the transporter is expressed than unmodified bacteria of the same bacterial subtype under the same conditions.

In one embodiment, the recombinant bacterial cells described herein comprise a first heterologous peptide transporter and a second heterologous peptide transporter. In one embodiment, said first peptide transporter is derived from a different organism than said second peptide transporter. In some embodiments, said first peptide transporter is derived from the same organism as said second peptide transporter. In some embodiments, said first peptide transporter imports the same peptide as said second peptide transporter. In other embodiment, said first peptide transporter imports a different peptide from said second peptide transporter. In some embodiments, said first peptide transporter is a wild-type peptide transporter and said second peptide transporter is a mutagenized version of said first peptide transporter. In some embodiments, the recombinant bacterial cells described herein comprise at least a third heterologous peptide transporter. In some embodiments, the recombinant bacterial cells described herein comprise at least four heterologous peptide transporters. In some embodiments, the recombinant bacterial cells described herein comprise at least five heterologous peptide transporters or more.

### Inducible Promoters

In some embodiments, the bacterial cell comprises a stably maintained plasmid or chromosome carrying the gene(s) encoding the transporter(s), such that the transporter(s) can be expressed in the host cell, and the host cell is capable of survival and/or growth in vitro, e.g., in medium, and/or in vivo, e.g., in the gut. In some embodiments, bacterial cell comprises two or more distinct transporters or operons, e.g., two or more transporter genes. In some embodiments, bacterial cell comprises three or more distinct transporters or operons, e.g., three or more transporter genes. In some embodiments, bacterial cell comprises 4, 5, 6, 7, 8, 9, 10, or more distinct transporters or operons, e.g., 4, 5, 6, 7, 8, 9, 10, or more transporter genes.

In some embodiments, the genetically engineered bacteria comprise multiple copies of the same transporter gene(s). In some embodiments, the gene encoding the transporter is present on a plasmid and operably linked to a directly or indirectly inducible promoter. In some embodiments, the gene encoding the transporter is present on a plasmid and operably linked to a promoter that is induced under low-oxygen or anaerobic conditions. In some embodiments, the gene encoding the transporter is present on a chromosome and operably linked to a directly or indirectly inducible promoter. In some embodiments, the gene encoding the transporter is present in the chromosome and operably linked to a promoter that is induced under low-oxygen or anaerobic conditions. In some embodiments, the gene encoding the transporter is present on a plasmid and operably linked to a promoter that is induced by exposure to tetracycline or arabinose.

In some embodiments, the promoter that is operably linked to the gene encoding the transporter is directly induced by exogenous environmental conditions. In some embodiments, the promoter that is operably linked to the gene encoding the transporter is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal. In some embodiments, the promoter is directly or indirectly induced by a molecule that is co-administered with the bacterial cell.

In certain embodiments, the bacterial cell comprises a gene encoding an transporter expressed under the control of a fumarate and nitrate reductase regulator (FNR) responsive promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive. FNR responsive promoters include, but are not limited to, the FNR responsive promoters listed in the chart, below. Underlined sequences are predicted ribosome binding sites, and bolded sequences are restriction sites used for cloning.

| FNR Responsive | |
|---|---|
| Promoter | Sequence |
| SEQ ID NO: | GTCAGCATAACACCCTGACCTCTCATTAATTGTTCATGCCGGGC |
| | GGCACTATCGTCGTCCGGCCTTTTCCTCTCTTACTCTGCTACGTA |
| | CATCTATTTCTATAAATCCGTTCAATTTGTCTGTTTTTTGCACAA |
| | ACATGAAATATCAGACAATTCCGTGACTTAAGAAAATTTATACA |
| | AATCAGCAATATACCCCTTAAGGAGTATATAAAGGTGAATTTGA |
| | TTTACATCAATAAGCGGGGTTGCTGAATCGTTAAGGTAGGCGGT |
| | AATAGAAAAGAAATCGAGGCAAAA |
| SEQ ID NO: | ATTTCCTCTCATCCCATCCGGGGTGAGAGTCTTTTCCCCCGACTT |
| | ATGGCTCATGCATGCATCAAAAAAGATGTGAGCTTGATCAAAA |
| | ACAAAAAATATTTCACTCGACAGGAGTATTTATATTGCGCCCGT |
| | TACGTGGGCTTCGACTGTAAATCAGAAAGGAGAAAACACCT |
| SEQ ID NO: | GTCAGCATAACACCCTGACCTCTCATTAATTGTTCATGCCGGGC |
| | GGCACTATCGTCGTCCGGCCTTTTCCTCTCTTACTCTGCTACGTA |
| | CATCTATTTCTATAAATCCGTTCAATTTGTCTGTTTTTTGCACAA |
| | ACATGAAATATCAGACAATTCCGTGACTTAAGAAAATTTATACA |
| | AATCAGCAATATACCCCTTAAGGAGTATATAAAGGTGAATTTGA |
| | TTTACATCAATAAGCGGGGTTGCTGAATCGTTAAGGATCCCTCT |
| | AGAAATAATTTTGTTTAACTTTAAGAAGGAGATATACAT |
| SEQ ID NO: | CATTTCCTCTCATCCCATCCGGGGTGAGAGTCTTTTCCCCCGACT |
| | TATGGCTCATGCATGCATCAAAAAAGATGTGAGCTTGATCAAAA |
| | ACAAAAAATATTTCACTCGACAGGAGTATTTATATTGCGCCCGG |
| | ATCCCTCTAGAAATAATTTTGTTTAACTTTAAGAAGGAGATATA |
| | CAT |
| SEQ ID NO: | AGTTGTTCTTATTGGTGGTGTTGCTTTATGGTTGCATCGTAGTAA |
| | ATGGTTGTAACAAAAGCAATTTTTCCGGCTGTCTGTATACAAAA |
| | ACGCCGTAAAGTTTGAGCGAAGTCAATAAACTCTCTACCCATTC |
| | AGGGCAATATCTCTCTTGGATCCCTCTAGAAATAATTTTGTTTA |
| | ACTTTAAGAAGGAGATATACAT |

In one embodiment, the FNR responsive promoter comprises SEQ ID NO:1. In another embodiment, the FNR responsive promoter comprises SEQ ID NO:2. In another embodiment, the FNR responsive promoter comprises SEQ ID NO:3. In another embodiment, the FNR responsive promoter comprises SEQ ID NO:4. In yet another embodiment, the FNR responsive promoter comprises SEQ ID NO:5.

In some embodiments, multiple distinct FNR nucleic acid sequences are inserted in the genetically engineered bacteria. In alternate embodiments, the genetically engineered bacteria comprise a gene encoding an transporter expressed under the control of an alternate oxygen level-dependent promoter, e.g., DNR (Trunk et al., 2010) or ANR (Ray et al., 1997). In these embodiments, expression of the transporter gene is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene encoding the transporter, in a low-oxygen or anaerobic environment, as compared to the native gene(s) and promoter in the bacteria under the same conditions. In certain embodiments, the non-native oxygen-level dependent transcriptional regulator is an FNR protein from N. gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene encoding the transporter, in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene encoding the transporter, in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., (2006).

In some embodiments, the bacterial cells comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene encoding the transporter are present on different plasmids. In some embodiments, the gene encoding the oxygen level-sensing transcriptional regulator and the gene encoding the transporter are present on the same plasmid. In some embodiments, the gene encoding the oxygen level-sensing transcriptional regulator is present on a chromosome. In some embodiments, the gene encoding the oxygen level-sensing transcriptional regulator and the gene encoding the transporter are present on different chromosomes. In some embodiments, the gene encoding the oxygen level-sensing transcriptional regulator and the gene encoding the transporter are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene encoding the transporter. In some embodiments, expression of the transcriptional regulator is controlled by the same promoter that controls expression of the transporter. In some embodiments, the transcriptional regulator and the transporter are divergently transcribed from a promoter region.

### RNS-Dependent Regulation

In some embodiments, the genetically engineered bacteria or genetically engineered virus comprise a gene encoding an transporter that is expressed under the control of an inducible promoter. In some embodiments, the genetically engineered bacterium or genetically engineered virus that expresses an transporter under the control of a promoter that is activated by inflammatory conditions. In one embodiment, the gene for producing the transporter is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO·), peroxynitrite or peroxynitrite anion (ONOO—), nitrogen dioxide (·NO2), dinitrogen trioxide (N2O3), peroxynitrous acid (ONOOH), and nitroperoxycarbonate (ONOOCO2-) (unpaired electrons denoted by ·). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene or genes, e.g., an transporter gene sequence(s), e.g., any of the transporters described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or genes, e.g., an transporter gene sequence(s). For example, in the presence of RNS, a transcription factor senses RNS and no longer binds to and/or represses the regulatory region, thereby derepressing an operatively linked gene sequence or gene cassette. Thus, RNS derepresses expression of the gene or genes.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 3.

### Examples of RNS-Sensing Transcription Factors and RNS-Responsive Genes

| RNS-sensing | Primarily | Examples of responsive |
|---|---|---|
| transcription | capable | genes, promoters, and/or |
| factor: | of sensing: | regulatory regions: |
| NsrR | NO | norB, aniA, nsrR, hmpA, ytfE, |
| | | ygbA, hcp, hcr, nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene or genes capable of directly or indirectly driving the expression of an transporter, thus controlling expression of the transporter relative to RNS levels. For example, the tunable regulatory region is a RNS-inducible regulatory region, and the payload is an transporter, such as any of the transporters provided herein; when RNS is present, e.g., in an inflamed tissue, a RNS-sensing transcription factor binds to and/or activates the regulatory region and drives expression of the transporter gene or genes. Subsequently, when inflammation is ameliorated, RNS levels are reduced, and production of the transporter is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or genes. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The genetically engineered bacteria of the invention may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012; Table 1). In certain embodiments, the genetically engineered bacteria of the invention comprise a RNS-inducible regulatory region from norVW that is operatively linked to a gene or genes, e.g., one or more transporter gene sequence(s). In the presence of RNS, a NorR transcription factor senses RNS and activates to the norVW regulatory region, thereby driving expression of the operatively linked gene(s) and producing the transporter.

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The genetically engineered bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008; Table 1). In certain embodiments, the genetically engineered bacteria of the invention comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or genes and producing one or more transporters. In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The genetically engineered bacteria of the invention may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010; Table 1). In certain embodiments, the genetically engineered bacteria of the invention comprise a RNS-derepressible regulatory region from norB that is operatively linked to a gene or genes, e.g., an transporter gene or genes. In the presence of RNS, an NsrR transcription factor senses RNS and no longer binds to the norB regulatory region, thereby derepressing the operatively linked an transporter gene or genes and producing the encoding an transporter(s).

In some embodiments, it is advantageous for the genetically engineered bacteria to express a RNS-sensing transcription factor that does not regulate the expression of a significant number of native genes in the bacteria. In some embodiments, the genetically engineered bacterium of the invention expresses a RNS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the genetically engineered bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the genetically engineered bacteria may comprise a two repressor activation regulatory circuit, which is used to express an transporter. The two repressor activation regulatory circuit comprises a first RNS-sensing repressor and a second repressor, which is operatively linked to a gene or gene cassette, e.g., encoding an transporter. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or genes. In the presence of binding by the first repressor (which occurs in the presence of RNS), expression of the second repressor is repressed, and the gene or genes, e.g., an transporter gene or genes is expressed.

A RNS-responsive transcription factor may induce, derepress, or repress gene expression depending upon the regulatory region sequence used in the genetically engineered bacteria. One or more types of RNS-sensing transcription factors and corresponding regulatory region sequences may be present in genetically engineered bacteria. In some embodiments, the genetically engineered bacteria comprise one type of RNS-sensing transcription factor, e.g., NsrR, and one corresponding regulatory region sequence, e.g., from norB. In some embodiments, the genetically engineered bacteria comprise one type of RNS-sensing transcription factor, e.g., NsrR, and two or more different corresponding regulatory region sequences, e.g., from norB and aniA. In some embodiments, the genetically engineered bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the genetically engineered bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the genetically engineered bacteria of the invention comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the genetically engineered bacteria of the invention comprise a gene for a RNS-sensing transcription factor from a different species, strain, or substrain of bacteria. In some embodiments, the genetically engineered bacteria comprise a RNS-responsive regulatory region from a different species, strain, or substrain of bacteria. In some embodiments, the genetically engineered bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the genetically engineered bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the genetically engineered bacteria of the invention comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the genetically engineered bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the transporter in the presence of RNS, as compared to the wild-type regulatory region under the same conditions. In some embodiments, the genetically engineered bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the transporter in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the transporter in the presence of RNS.

In some embodiments, the gene or gene cassette for producing the anti-inflammation and/or gut barrier function enhancer molecule is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of one or more encoding an transporter gene(s) may be integrated into the bacterial chromosome. Having multiple copies of the gene or gen(s) integrated into the chromosome allows for greater production of the transporter(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

### ROS-Dependent Regulation

In some embodiments, the genetically engineered bacteria or genetically engineered virus comprise a gene for producing an transporter that is expressed under the control of an inducible promoter. In some embodiments, the genetically engineered bacterium or genetically engineered virus that expresses an transporter under the control of a promoter that is activated by conditions of cellular damage. In one embodiment, the gene for producing the transporter is expressed under the control of an cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H₂O₂), organic peroxide (ROOH), hydroxyl ion (OH—), hydroxyl radical (·OH), superoxide or superoxide anion (·O2-), singlet oxygen (1O2), ozone (O3), carbonate radical, peroxide or peroxyl radical (·O2-2), hypochlorous acid (HOCl), hypochlorite ion (OCl—), sodium hypochlorite (NaOCl), nitric oxide (NO·), and peroxynitrite or peroxynitrite anion (ONOO—) (unpaired electrons denoted by ·). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more transporter(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or genes.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or genes, e.g., one or more genes encoding one or more transporter(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or genes.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 4.

### Examples of ROS-Sensing Transcription Factors and ROS-Responsive Genes

| ROS-sensing | Primarily | Examples of responsive |
|---|---|---|
| transcription | capable | genes, promoters, and/or |
| factor: | of sensing: | regulatory regions: |
| OxyR | H₂O₂ | ahpC; ahpF; dps; dsbG; fhuF; flu; fur; |
| | | gor; grxA; hemH; katG; oxyS; sufA; |
| | | sufB; sufC; sufD; sufE; sufS; trxC; |
| | | uxuA; yaaA; yaeH; yaiA; ybjM; ydcH; |
| | | ydeN; ygaQ; yljA; ytfK |
| PerR | H₂O₂ | katA; ahpCF; mrgA; zoaA; fur; |
| | | hemAXCDBL; srfA |
| OhrR | Organic peroxides | ohrA |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; |
| | | tnp2d; phoD; tnp15b; pstA; tnp5b; |
| | | xylC; gabD1; rluC2; cgtS9; azlC; |
| | | narKGHJI; rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of an transporter, thus controlling expression of the transporter relative to ROS levels. For example, the tunable regulatory region is a ROS-inducible regulatory region, and the molecule is an transporter; when ROS is present, e.g., in an inflamed tissue, a ROS-sensing transcription factor binds to and/or activates the regulatory region and drives expression of the gene sequence for the transporter, thereby producing the transporter. Subsequently, when inflammation is ameliorated, ROS levels are reduced, and production of the transporter is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H₂O₂ detoxification (katE, ahpCF), heme biosynthesis (hemH), reductant supply (grxA, gor, trxC), thiol-disulfide isomerization (dsbG), Fe—S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012; Table 1). In certain embodiments, the genetically engineered bacteria of the invention comprise a ROS-inducible regulatory region from oxyS that is operatively linked to a gene, e.g., an transporter gene. In the presence of ROS, e.g., H2O2, an OxyR transcription factor senses ROS and activates to the oxyS regulatory region, thereby driving expression of the operatively linked transporter gene and producing the transporter. In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The genetically engineered bacteria of the invention may comprise any suitable ROS-responsive regulatory region from a gene that is activated by SoxR. Genes that are capable of being activated by SoxR are known in the art (see, e.g., Koo et al., 2003; Table 1). In certain embodiments, the genetically engineered bacteria of the invention comprise a ROS-inducible regulatory region from soxS that is operatively linked to a gene, e.g., an transporter. In the presence of ROS, the SoxR transcription factor senses ROS and activates the soxS regulatory region, thereby driving expression of the operatively linked an transporter gene and producing the an transporter.

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H₂O₂ but it shows much higher reactivity for organic hydroperoxides” (Duarte et al., 2010). The genetically engineered bacteria of the invention may comprise any suitable ROS-responsive regulatory region from a gene that is repressed by OhrR. Genes that are capable of being repressed by OhrR are known in the art (see, e.g., Dubbs et al., 2012; Table 1). In certain embodiments, the genetically engineered bacteria of the invention comprise a ROS-derepressible regulatory region from ohrA that is operatively linked to a gene or gene cassette, e.g., an transporter gene. In the presence of ROS, e.g., NaOCl, an OhrR transcription factor senses ROS and no longer binds to the ohrA regulatory region, thereby derepressing the operatively linked transporter gene and producing the an transporter.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the genetically engineered bacteria of the invention comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA” and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The genetically engineered bacteria of the invention may comprise any suitable ROS-responsive regulatory region from a gene that is repressed by RosR. Genes that are capable of being repressed by RosR are known in the art (see, e.g., Bussmann et al., 2010; Table 1). In certain embodiments, the genetically engineered bacteria of the invention comprise a ROS-derepressible regulatory region from cgtS9 that is operatively linked to a gene or gene cassette, e.g., an transporter. In the presence of ROS, e.g., H2O2, a RosR transcription factor senses ROS and no longer binds to the cgtS9 regulatory region, thereby derepressing the operatively linked transporter gene and producing the transporter.

In some embodiments, it is advantageous for the genetically engineered bacteria to express a ROS-sensing transcription factor that does not regulate the expression of a significant number of native genes in the bacteria. In some embodiments, the genetically engineered bacterium of the invention expresses a ROS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the genetically engineered bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA) residing within and near the promoter sequences of PerR-controlled genes” (Marinho et al., 2014). PerR is capable of binding a regulatory region that “overlaps part of the promoter or is immediately downstream from it” (Dubbs et al., 2012). The genetically engineered bacteria of the invention may comprise any suitable ROS-responsive regulatory region from a gene that is repressed by PerR. Genes that are capable of being repressed by PerR are known in the art (see, e.g., Dubbs et al., 2012; Table 1).

In these embodiments, the genetically engineered bacteria may comprise a two repressor activation regulatory circuit, which is used to express an transporter. The two repressor activation regulatory circuit comprises a first ROS-sensing repressor, e.g., PerR, and a second repressor, e.g., TetR, which is operatively linked to a gene or gene cassette, e.g., an transporter. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., an transporter. In the absence of PerR binding (which occurs in the absence of ROS), tetR is transcribed, and TetR represses expression of the gene or gene cassette, e.g., an transporter. In the presence of PerR binding (which occurs in the presence of ROS), tetR expression is repressed, and the gene or gene cassette, e.g., an transporter, is expressed.

A ROS-responsive transcription factor may induce, derepress, or repress gene expression depending upon the regulatory region sequence used in the genetically engineered bacteria. For example, although “OxyR is primarily thought of as a transcriptional activator under oxidizing conditions. . . . OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The genetically engineered bacteria of the invention may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 1). Or, for example, although RosR is capable of repressing a number of genes, it is also capable of activating certain genes, e.g., the narKGHJI operon. In some embodiments, the genetically engineered bacteria comprise any suitable ROS-responsive regulatory region from a gene that is activated by RosR. In addition, “PerR-mediated positive regulation has also been observed . . . and appears to involve PerR binding to distant upstream sites” (Dubbs et al., 2012). In some embodiments, the genetically engineered bacteria comprise any suitable ROS-responsive regulatory region from a gene that is activated by PerR.

One or more types of ROS-sensing transcription factors and corresponding regulatory region sequences may be present in genetically engineered bacteria. For example, “OhrR is found in both Gram-positive and Gram-negative bacteria and can coreside with either OxyR or PerR or both” (Dubbs et al., 2012). In some embodiments, the genetically engineered bacteria comprise one type of ROS-sensing transcription factor, e.g., OxyR, and one corresponding regulatory region sequence, e.g., from oxyS. In some embodiments, the genetically engineered bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the genetically engineered bacteria comprise two or more types of ROS-sensing transcription factors and one corresponding regulatory region sequence.

Nucleic acid sequences of several exemplary OxyR-regulated regulatory regions are shown in Table 5. OxyR binding sites are underlined and bolded. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 46, 47, 48, or 49, or a functional fragment thereof.

### Nucleotide Sequences of Exemplary OxyR-Regulated Regulatory Regions

| Regulatory | |
|---|---|
| sequence | 01234567890123456789012345678901234567890123456789 |
| katG | TGTGGCTTTTATGAAAATCACACAGTGATCACAAATTTTAAACA |
| (SEQ ID NO:) | GAGCACAAAATGCTGCCTCGAAATGAGGGCGGGAAAATAAGGT |
| | TATCAGCCTTGTTTTCTCCCTCATTACTTGAAGGATATGAAGCTA |
| | AAACCCTTTTTTATAAAGCATTTGTCCGAATTCGGACATAATCA |
| | AAAAAGCTTAATTAAGATCAATTTGATCTACATCTCTTTAACCA |
| | ACAATATGTAAGATCTCAACTATCGCATCCGTGGATTAATTC |
| | AATTATAACTTCTCTCTAACGCTGTGTATCGTAACGGTAACACT |
| | GTAGAGGGGAGCACATTGATGCGAATTCATTAAAGAGGAGAAA |
| | GGTACC |
| dps | TTCCGAAAATTCCTGGCGAGCAGATAAATAAGAATTGTTCTTAT |
| (SEQ ID NO:) | CAATATATCTAACTCATTGAATCTTTATTAGTTTTGTTTTTCACG |
| | CTTGTTACCACTATTAGTGTGATAGGAACAGCCAGAATAGCG |
| | GAACACATAGCCGGTGCTATACTTAATCTCGTTAATTACTGGGA |
| | CATAACATCAAGAGGATATGAAATTCGAATTCATTAAAGAGGA |
| | GAAAGGTACC |
| ahpC | GCTTAGATCAGGTGATTGCCCTTTGTTTATGAGGGTGTTGTAATC |
| (SEQ ID NO:) | CATGTCGTTGTTGCATTTGTAAGGGCAACACCTCAGCCTGCAGG |
| | CAGGCACTGAAGATACCAAAGGGTAGTTCAGATTACACGGTCA |
| | CCTGGAAAGGGGGCCATTTTACTTTTTATCGCCGCTGGCGGTGC |
| | AAAGTTCACAAAGTTGTCTTACGAAGGTTGTAAGGTAAAACTT |
| | ATCGATTTGATAATGGAAACGCATTAGCCGAATCGGCAAAAAT |
| | TGGTTACCTTACATCTCATCGAAAACACGGAGGAAGTATAGATG |
| | CGAATTCATTAAAGAGGAGAAAGGTACC |
| oxyS | CTCGAGTTCATTATCCATCCTCCATCGCCACGATAGTTCATGGC |
| (SEQ ID NO:) | GATAGGTAGAATAGCAATGAACGATTATCCCTATCAAGCATTC |
| | TGACTGATAATTGCTCACACGAATTCATTAAAGAGGAGAAAGGT |
| | ACC |

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the genetically engineered bacteria of the invention comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the genetically engineered bacteria of the invention comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the transporter in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the transporter in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the transporter in the presence of ROS.

In some embodiments, the gene or gene cassette for producing the transporter is present on a plasmid and operably linked to a promoter that is induced by ROS. In some embodiments, the gene or gene cassette for producing the transporter is present in the chromosome and operably linked to a promoter that is induced by ROS. In some embodiments, the gene or gene cassette for producing the transporter is present on a chromosome and operably linked to a promoter that is induced by exposure to tetracycline. In some embodiments, the gene or gene cassette for producing the transporter is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing an transporter(s). In some embodiments, the gene(s) capable of producing an transporter(s) is present on a plasmid and operatively linked to a ROS-responsive regulatory region. In some embodiments, the gene(s) capable of producing an transporter is present in a chromosome and operatively linked to a ROS-responsive regulatory region.

Thus, in some embodiments, the genetically engineered bacteria or genetically engineered virus produce one or more transporters under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing an transporter, such that the transporter can be expressed in the host cell, and the host cell is capable of survival and/or growth in vitro, e.g., in medium, and/or in vivo. In some embodiments, a bacterium may comprise multiple copies of the gene encoding the transporter. In some embodiments, the gene encoding the transporter is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene encoding the transporter is expressed on a high-copy plasmid. In some embodiments, the high-copy plasmid may be useful for increasing expression of the transporter. In some embodiments, the gene encoding the transporter is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene encoding a particular transporter inserted at four different insertion sites. Alternatively, the genetically engineered bacteria may include three copies of the gene encoding a particular transporter inserted at three different insertion sites and three copies of the gene encoding a different transporter inserted at three different insertion sites.

In some embodiments, under conditions where the transporter is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the transporter, and/or transcript of the gene(s) in the operon as compared to unmodified bacteria of the same subtype under the same conditions.

In some embodiments, quantitative PCR (qPCR) is used to amplify, detect, and/or quantify mRNA expression levels of the transporter gene(s). Primers specific for transporter the gene(s) may be designed and used to detect mRNA in a sample according to methods known in the art. In some embodiments, a fluorophore is added to a sample reaction mixture that may contain transporter mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the transporter gene(s).

In some embodiments, quantitative PCR (qPCR) is used to amplify, detect, and/or quantify mRNA expression levels of the transporter gene(s). Primers specific for transporter the gene(s) may be designed and used to detect mRNA in a sample according to methods known in the art. In some embodiments, a fluorophore is added to a sample reaction mixture that may contain transporter mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the transporter gene(s).

### Essential Genes and Auxotrophs

As used herein, the term “essential gene” refers to a gene that is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37: D455-D458 and Gerdes et al., Essential genes on metabolic maps, Curr. Opin. Biotechnol., 17 (5): 448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in a gene required for cell survival and/or growth. In one embodiment, the essential gene is a DNA synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria. For example, thymine is a nucleic acid that is required for bacterial cell growth; in its absence, bacteria undergo cell death. The thyA gene encodes thimidylate synthetase, an enzyme that catalyzes the first step in thymine synthesis by converting dUMP to dTMP (Sat et al. (2003) J Bacteriol. (2003) 185 (6): 1803-7). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro. Without sufficient amounts of thymine, the thyA auxotroph dies. In some embodiments, the auxotrophic modification is used to ensure that the bacterial cell does not survive in the absence of the auxotrophic gene product, e.g., outside of the hypoxic tumor environment.

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which the dapD gene is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product.

In other embodiments, the genetically engineered bacterium of the present disclosure is a uraA auxotroph in which the uraA gene is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product.

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria of the invention comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, IpxH, cysS, fold, rplT, infC, thrS, nadE, gapA, yeaZ, aspS, argS, pgsA, yefM, metG, folE, yejM, gyrA, nrdA, nrdB, folC, accD, fabB, gliX, ligA, zipA, dapE, dapA, der, hisS, ispG, suhB, tadA, acpS, era, rnc, ftsB, eno, pyrG, chpR, Igt, fbaA, pgk, yqgD, metK, yqgF, plsC, ygiT, pare, ribB, cca, ygjD, tdcF, yraL, yihA, fisN, murl, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, IspA, ispH, dapB, folA, imp, yabQ, ftsL, ftsl, murE, murF, mraY, murD, ftsW, murG, murC, ftsQ, ftsA, ftsZ, lpxC, secM, secA, can, folk, hemL, yadR, dapD, map, rpsB, infB, nusA, fisH, obgE, rpmA, rplU, ispB, murA, yrbB, yrbK, yhbN, rpsl, rplM, degS, mreD, mreC, mreB, accB, accC, yrdC, def, fint, rplQ, rpoA, rpsD, rpsK, rpsM, entD, mrdB, mrdA, nadD, hlepB, rpoE, pssA, yfiO, rplS, trmD, rpsP, ffh, grpE, yfjB, csrA, ispF, ispD, rplW, rplD, rplC, rpsJ, fusA, rpsG, rpsL, trpS, yrfF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, IpxD, fabZ, IpxA, IpxB, dnaE, accA, tilS, proS, yafF, tsf, pyrH, olA, rlpB, leuS, Int, glnS, fldA, cydA, infA, cydC, ftsK, lolA, serS, rpsA, msbA, lpxK, kdsB, mukF, mukE, mukB, asnS, fabA, mviN, rne, yceQ, fabD, fabG, acpP, tmk, holB, lolC, lolD, lolE, purB, ymfK, minE, mind, pth, rsA, ispE, lolB, hemA, prfA, prmC, kdsA, topA, ribA, fabl, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLIDE) bacterial cell. SLIDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain, “ACS Synthetic Biology (2015) DOI: 10.1021/acssynbio.5b00085, the entire contents of which are expressly incorporated herein by reference).

In some embodiments, the SLIDE bacterial cell comprises a mutation in an essential gene. In some embodiments, the essential gene is selected from the group consisting of pheS, dnaN, tyrS, metG, and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, 1317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A, and F40G. In some embodiments, the essential gene is tyrS comprising the mutations L36V, C38A, and F40G. In some embodiments, the essential gene is metG comprising one or more of the following mutations: E45Q, N47R, 149G, and A51C. In some embodiments, the essential gene is metG comprising the mutations E45Q, N47R, 149G, and A51C. In some embodiments, the essential gene is adk comprising one or more of the following mutations: I4L, L5I, and L6G. In some embodiments, the essential gene is adk comprising the mutations I4L, L5I, and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, 149G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L51, and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, 149G, and A51C). In other embodiments, the bacterial cell comprises mutations in three essential genes. For example, in some embodiments, the bacterial cell comprises mutations in tyrS (L36V, C38A, and F40G), metG (E45Q, N47R, 149G, and A51C), and pheS (F125G, P183T, P184A, R186A, and I188L).

In some embodiments, the genetically engineered bacterium is a conditional auxotroph whose essential gene(s) is replaced using an arabinose system.

In some embodiments, the genetically engineered bacterium of the disclosure is an auxotroph and also comprises kill switch circuitry, such as any of the kill switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA and may also comprise a toxin gene that is regulated by one or more transcriptional activators that are expressed in response to an environmental condition(s) and/or signal(s) (such as low oxygen levels) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4:307-16, the entire contents of which are expressly incorporated herein by reference). In some embodiments, the genetically engineered bacterium of the disclosure is an auxotroph and also comprises kill switch circuitry, such as any of the kill switch components and systems described herein, as well as another biosecurity system, such a conditional origin of replication (see Wright et al., supra). In other embodiments, auxotrophic modifications may also be used to screen for mutant bacteria that produce the substrate transporter.

### Genetic Regulatory Circuits

In some embodiments, the genetically engineered bacteria comprise multilayered genetic regulatory circuits for expressing the constructs described herein (see, e.g., U.S. Provisional Application No. 62/184,811, incorporated herein by reference in its entirety). The genetic regulatory circuits are useful to screen for mutant bacteria that produce a substrate transporter or rescue an auxotroph. In certain embodiments, the invention provides methods for selecting genetically engineered bacteria that produce one or more genes of interest.

In some embodiments, the invention provides genetically engineered bacteria comprising a gene or gene cassette for producing a payload and a T7 polymerase-regulated genetic regulatory circuit. For example, the genetically engineered bacteria comprise a first gene encoding a T7 polymerase, wherein the first gene is operably linked to a fumarate and nitrate reductase regulator (FNR)-responsive promoter; a second gene or gene cassette for producing a payload, wherein the second gene or gene cassette is operably linked to a T7 promoter that is induced by the T7 polymerase; and a third gene encoding an inhibitory factor, lys Y, that is capable of inhibiting the T7 polymerase. In the presence of oxygen, FNR does not bind the FNR-responsive promoter, and the payload is not expressed. LysY is expressed constitutively (P-lac constitutive) and further inhibits T7 polymerase. In the absence of oxygen, FNR dimerizes and binds to the FNR-responsive promoter, T7 polymerase is expressed at a level sufficient to overcome lysY inhibition, and the payload is expressed. In some embodiments, the lysY gene is operably linked to an additional FNR binding site. In the absence of oxygen, FNR dimerizes to activate T7 polymerase expression as described above, and also inhibits lys Y expression.

In some embodiments, the invention provides genetically engineered bacteria comprising a gene or gene cassette for producing a payload, and a protease-regulated genetic regulatory circuit. For example, the genetically engineered bacteria comprise a first gene encoding an mf-lon protease, wherein the first gene is operably linked to a FNR-responsive promoter; a second gene or gene cassette for producing a payload operably linked to a tet regulatory region (tetO); and a third gene encoding an mf-lon degradation signal linked to a tet repressor (tetR), wherein the tetR is capable of binding to the tet regulatory region and repressing expression of the second gene or gene cassette. The mf-lon protease is capable of recognizing the mf-lon degradation signal and degrading the tetR. In the presence of oxygen, FNR does not bind the FNR-responsive promoter, the repressor is not degraded, and the payload is not expressed. In the absence of oxygen, FNR dimerizes and binds the FNR-responsive promoter, thereby inducing expression of mf-lon protease. The mf-lon protease recognizes the mf-lon degradation signal and degrades the tetR, and the payload is expressed.

In some embodiments, the invention provides genetically engineered bacteria comprising a gene or gene cassette for producing a payload, and a repressor-regulated genetic regulatory circuit. For example, the genetically engineered bacteria comprise a first gene encoding a first repressor, wherein the first gene is operably linked to a FNR-responsive promoter; a second gene or gene cassette for producing a payload operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the payload is not expressed. In the absence of oxygen, FNR dimerizes and binds the FNR-responsive promoter, the first repressor is expressed, the second repressor is not expressed, and the payload is expressed.

Examples of repressors useful in these embodiments include, but are not limited to, ArgR, TetR, ArsR, AscG, LacI, CscR, DeoR, DgoR, FruR, GalR, GatR, CI, LexA, RafR, QacR, and PtxS (US20030166191).

In some embodiments, the invention provides genetically engineered bacteria comprising a gene or gene cassette for producing a payload and a regulatory RNA-regulated genetic regulatory circuit. For example, the genetically engineered bacteria comprise a first gene encoding a regulatory RNA, wherein the first gene is operably linked to a FNR-responsive promoter, and a second gene or gene cassette for producing a payload. The second gene or gene cassette is operably linked to a constitutive promoter and further linked to a nucleotide sequence capable of producing an mRNA hairpin that inhibits translation of the payload. The regulatory RNA is capable of eliminating the mRNA hairpin and inducing payload translation via the ribosomal binding site. In the presence of oxygen, FNR does not bind the FNR-responsive promoter, the regulatory RNA is not expressed, and the mRNA hairpin prevents the payload from being translated. In the absence of oxygen, FNR dimerizes and binds the FNR-responsive promoter, the regulatory RNA is expressed, the mRNA hairpin is eliminated, and the payload is expressed.

In some embodiments, the invention provides genetically engineered bacteria comprising a gene or gene cassette for producing a payload, and a CRISPR-regulated genetic regulatory circuit. For example, the genetically engineered bacteria comprise a Cas9 protein; a first gene encoding a CRISPR guide RNA, wherein the first gene is operably linked to a FNR-responsive promoter; a second gene or gene cassette for producing a payload, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the payload is not expressed. In the absence of oxygen, FNR dimerizes and binds the FNR-responsive promoter, the guide RNA is expressed, the repressor is not expressed, and the payload is expressed.

In some embodiments, the invention provides genetically engineered bacteria comprising a gene or gene cassette for producing a payload and a recombinase-regulated genetic regulatory circuit. For example, the genetically engineered bacteria comprise a first gene encoding a recombinase, wherein the first gene is operably linked to a FNR-responsive promoter, and a second gene or gene cassette for producing a payload operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the payload remains in the 3′ to 5′ orientation, and no functional payload is produced. In the absence of oxygen, FNR dimerizes and binds the FNR-responsive promoter, the recombinase is expressed, the payload is reverted to the 5′ to 3′ orientation, and functional payload is produced.

In some embodiments, the invention provides genetically engineered bacteria comprising a gene or gene cassette for producing a payload and a polymerase- and recombinase-regulated genetic regulatory circuit. For example, the genetically engineered bacteria comprise a first gene encoding a recombinase, wherein the first gene is operably linked to a FNR-responsive promoter; a second gene or gene cassette for producing a payload operably linked to a T7 promoter; a third gene encoding a T7 polymerase, wherein the T7 polymerase is capable of binding to the T7 promoter and inducing expression of the payload. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the payload is not expressed. In the absence of oxygen, FNR dimerizes and binds the FNR-responsive promoter, the recombinase is expressed, the T7 polymerase gene is reverted to the 5′ to 3′ orientation, and the payload is expressed.

### Host-Plasmid Mutual Dependency

In some embodiments, the genetically engineered bacteria of the invention also comprise a plasmid that has been modified to create a host-plasmid mutual dependency. In certain embodiments, the mutually dependent host-plasmid platform is GeneGuard (Wright et al., 2015). In some embodiments, the GeneGuard plasmid comprises (i) a conditional origin of replication, in which the requisite replication initiator protein is provided in trans; (ii) an auxotrophic modification that is rescued by the host via genomic translocation and is also compatible for use in rich media; and/or (iii) a nucleic acid sequence which encodes a broad-spectrum toxin. The toxin gene may be used to select against plasmid spread by making the plasmid DNA itself disadvantageous for strains not expressing the anti-toxin (e.g., a wild-type bacterium). In some embodiments, the GeneGuard plasmid is stable for at least 100 generations without antibiotic selection. In some embodiments, the GeneGuard plasmid does not disrupt growth of the host. The GeneGuard plasmid is used to greatly reduce unintentional plasmid propagation in the genetically engineered bacteria of the invention.

The mutually dependent host-plasmid platform may be used alone or in combination with other biosafety mechanisms, such as those described herein (e.g., kill switches, auxotrophies). In some embodiments, the genetically engineered bacteria comprise a GeneGuard plasmid. In other embodiments, the genetically engineered bacteria comprise a GeneGuard plasmid and/or one or more kill switches. In other embodiments, the genetically engineered bacteria comprise a GeneGuard plasmid and/or one or more auxotrophies. In still other embodiments, the genetically engineered bacteria comprise a GeneGuard plasmid, one or more kill switches, and/or one or more auxotrophies.

### Kill Switch

In some embodiments, the genetically engineered bacteria of the invention also comprise a kill switch (see, e.g., U.S. Provisional Application Nos. 62/183,935 and 62/263,329 incorporated herein by reference in their entireties). The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

Bacteria engineered with kill switches have been engineered for in vitro research purposes, e.g., to limit the spread of a biofuel-producing microorganism outside of a laboratory environment. Bacteria engineered for in vivo administration to treat a disease or disorder may also be programmed to die at a specific time after the expression and delivery of a heterologous gene or genes, for example, a therapeutic gene(s) or after the subject has experienced the therapeutic effect. For example, in some embodiments, the kill switch is activated to kill the bacteria after a period of time following oxygen level-dependent expression of the substrate transporter. In some embodiments, the kill switch is activated in a delayed fashion following oxygen level-dependent expression of the substrate transporter. Alternatively, the bacteria may be engineered to die if the bacteria have spread outside of a target site (e.g., a tumor site). Specifically, it may be useful to prevent the spread of the microorganism outside the area of interest (for example, outside of the tumor site) within the subject, or spread of the microorganism outside of the subject into the environment (for example, spread to the environment through the blood or stool of the subject). Examples of such toxins that can be used in kill switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al. (2000) Nature 403:339-42), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al. (2010) Proc. Natl. Acad. Sci. USA 107 (36): 15898-903.). In some embodiments, the kill switch is activated to kill the bacteria after a period of time following oxygen level-dependent expression of the substrate transporter. In some embodiments, the kill switch is activated in a delayed fashion following oxygen level-dependent expression of the substrate transporter.

Kill switches can be designed such that a toxin is produced in response to an environmental condition or external signal (e.g., the bacteria is killed in response to an external cue) or, alternatively designed such that a toxin is produced once an environmental condition no longer exists or an external signal is ceased.

Thus, in some embodiments, the genetically engineered bacteria of the disclosure are further programmed to die after sensing an exogenous environmental signal, for example, in a low oxygen environment. In some embodiments, the genetically engineered bacteria of the present disclosure comprise one or more genes encoding one or more recombinase(s), whose expression is induced in response to an environmental condition or signal and causes one or more recombination events that ultimately leads to the expression of a toxin which kills the cell. In some embodiments, the at least one recombination event is the flipping of an inverted heterologous gene encoding a bacterial toxin which is then constitutively expressed after it is flipped by the first recombinase. In one embodiment, constitutive expression of the bacterial toxin kills the genetically engineered bacterium. In these types of kill switch systems, once the engineered bacterial cell senses the exogenous environmental condition and expresses the heterologous gene of interest, the recombinant bacterial cell is no longer viable.

In another embodiment in which the genetically engineered bacteria of the present disclosure express one or more recombinase(s) in response to an environmental condition or signal causing at least one recombination event, the genetically engineered bacterium further expresses a heterologous gene encoding an anti-toxin in response to an exogenous environmental condition or signal. In one embodiment, the at least one recombination event is flipping of an inverted heterologous gene encoding a bacterial toxin by a first recombinase. In one embodiment, the inverted heterologous gene encoding the bacterial toxin is located between a first forward recombinase recognition sequence and a first reverse recombinase recognition sequence. In one embodiment, the heterologous gene encoding the bacterial toxin is constitutively expressed after it is flipped by the first recombinase. In one embodiment, the anti-toxin inhibits the activity of the toxin, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In another embodiment, the at least one recombination event is flipping of an inverted heterologous gene encoding a second recombinase by a first recombinase, followed by the flipping of an inverted heterologous gene encoding a bacterial toxin by the second recombinase. In one embodiment, the inverted heterologous gene encoding the second recombinase is located between a first forward recombinase recognition sequence and a first reverse recombinase recognition sequence. In one embodiment, the inverted heterologous gene encoding the bacterial toxin is located between a second forward recombinase recognition sequence and a second reverse recombinase recognition sequence. In one embodiment, the heterologous gene encoding the second recombinase is constitutively expressed after it is flipped by the first recombinase. In one embodiment, the heterologous gene encoding the bacterial toxin is constitutively expressed after it is flipped by the second recombinase. In one embodiment, the genetically engineered bacterium is killed by the bacterial toxin. In one embodiment, the genetically engineered bacterium further expresses a heterologous gene encoding an anti-toxin in response to the exogenous environmental condition. In one embodiment, the anti-toxin inhibits the activity of the toxin when the exogenous environmental condition is present, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In one embodiment, the at least one recombination event is flipping of an inverted heterologous gene encoding a second recombinase by a first recombinase, followed by flipping of an inverted heterologous gene encoding a third recombinase by the second recombinase, followed by flipping of an inverted heterologous gene encoding a bacterial toxin by the third recombinase.

In one embodiment, the at least one recombination event is flipping of an inverted heterologous gene encoding a first excision enzyme by a first recombinase. In one embodiment, the inverted heterologous gene encoding the first excision enzyme is located between a first forward recombinase recognition sequence and a first reverse recombinase recognition sequence. In one embodiment, the heterologous gene encoding the first excision enzyme is constitutively expressed after it is flipped by the first recombinase. In one embodiment, the first excision enzyme excises a first essential gene. In one embodiment, the programmed recombinant bacterial cell is not viable after the first essential gene is excised.

In one embodiment, the first recombinase further flips an inverted heterologous gene encoding a second excision enzyme. In one embodiment, the wherein the inverted heterologous gene encoding the second excision enzyme is located between a second forward recombinase recognition sequence and a second reverse recombinase recognition sequence. In one embodiment, the heterologous gene encoding the second excision enzyme is constitutively expressed after it is flipped by the first recombinase. In one embodiment, the genetically engineered bacterium dies or is no longer viable when the first essential gene and the second essential gene are both excised. In one embodiment, the genetically engineered bacterium dies or is no longer viable when either the first essential gene is excised or the second essential gene is excised by the first recombinase.

In one embodiment, the genetically engineered bacterium dies after the at least one recombination event occurs. In another embodiment, the genetically engineered bacterium is no longer viable after the at least one recombination event occurs.

In any of these embodiment, the recombinase can be a recombinase selected from the group consisting of: BxbI, PhiC31, TP901, BxbI, PhiC31, TP901, HK022, HP1, R4, Int1, Int2, Int3, Int4, Int5, Int6, Int7, Int8, Int9, Int10, Int11, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34, or a biologically active fragment thereof.

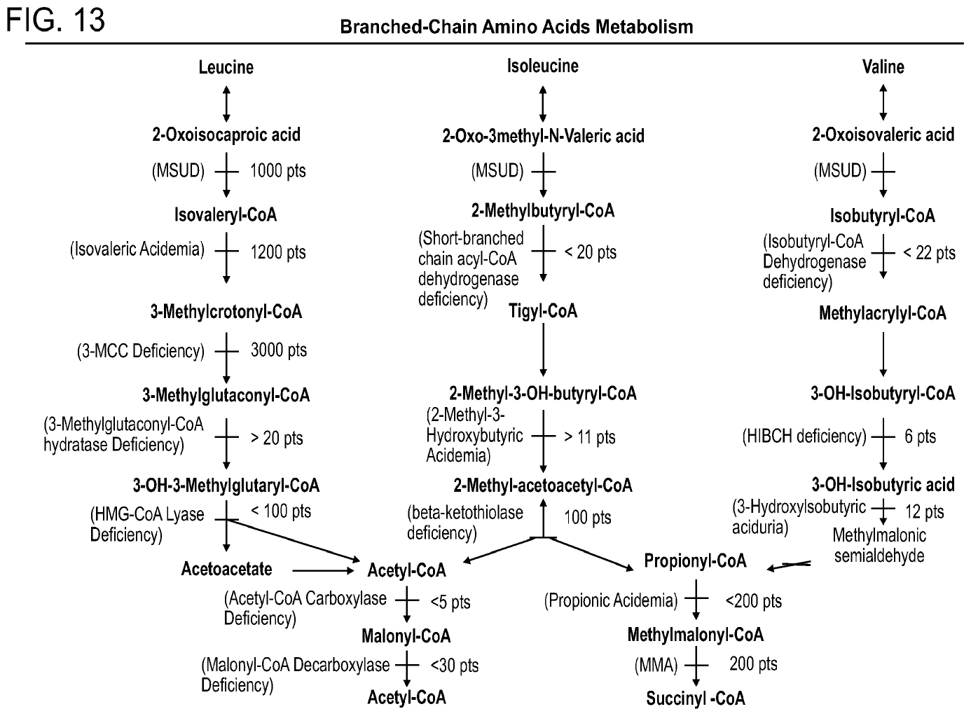

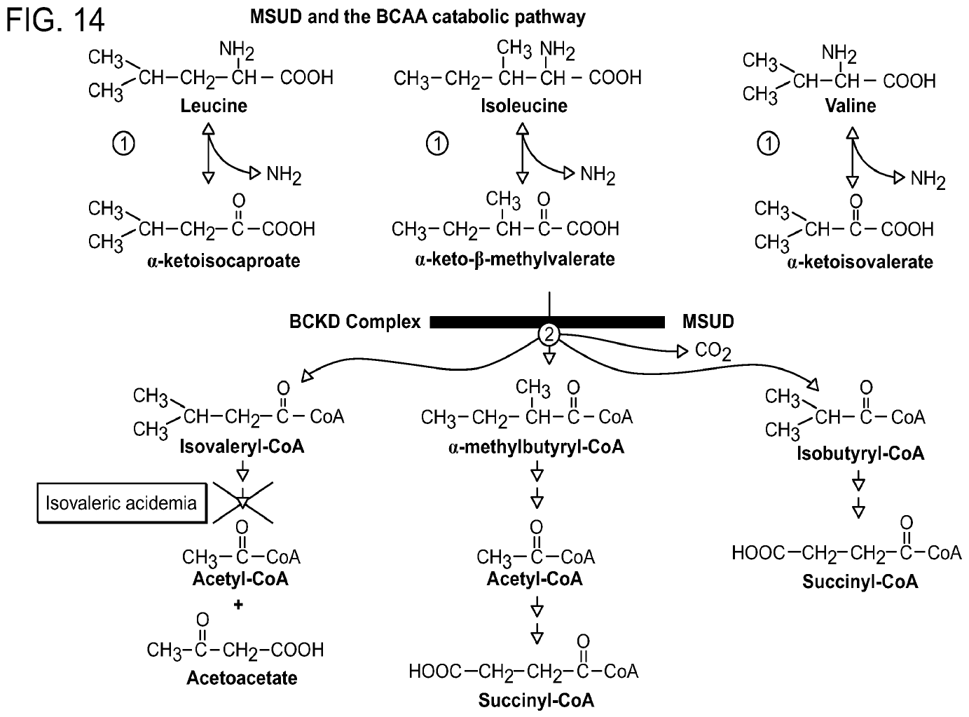

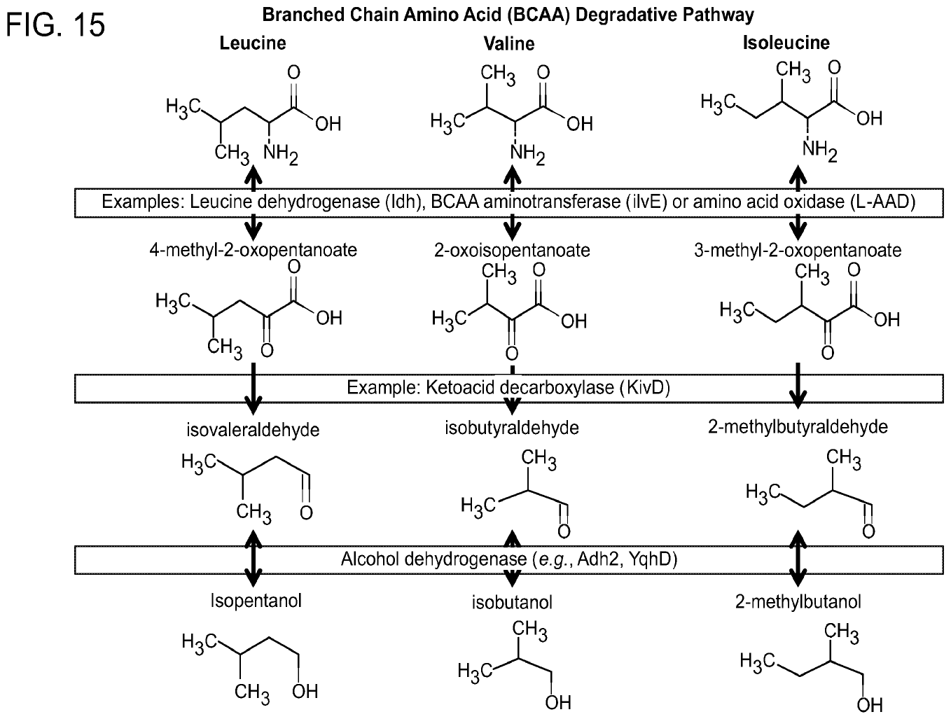

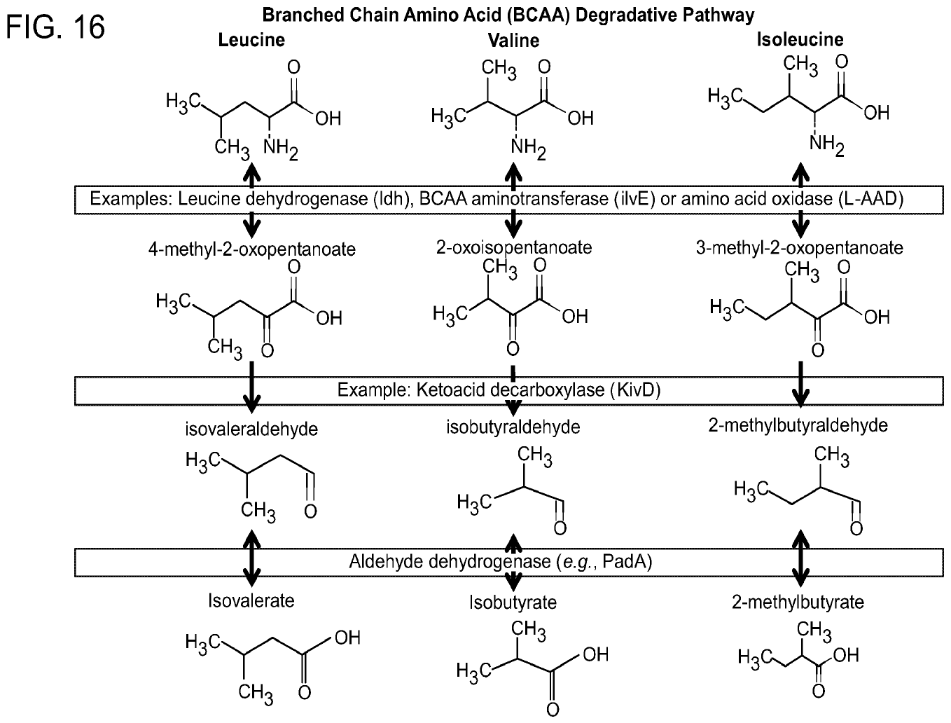

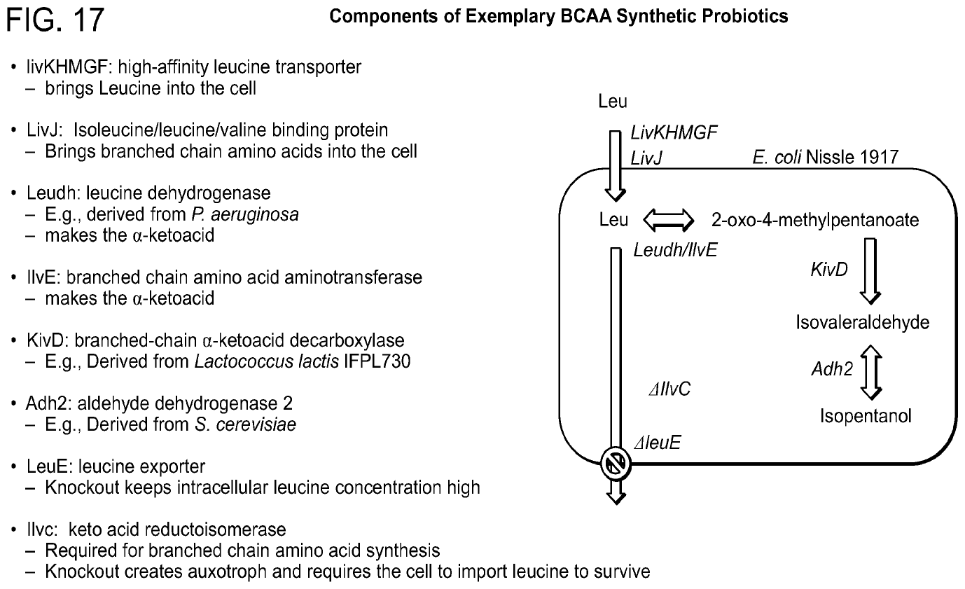

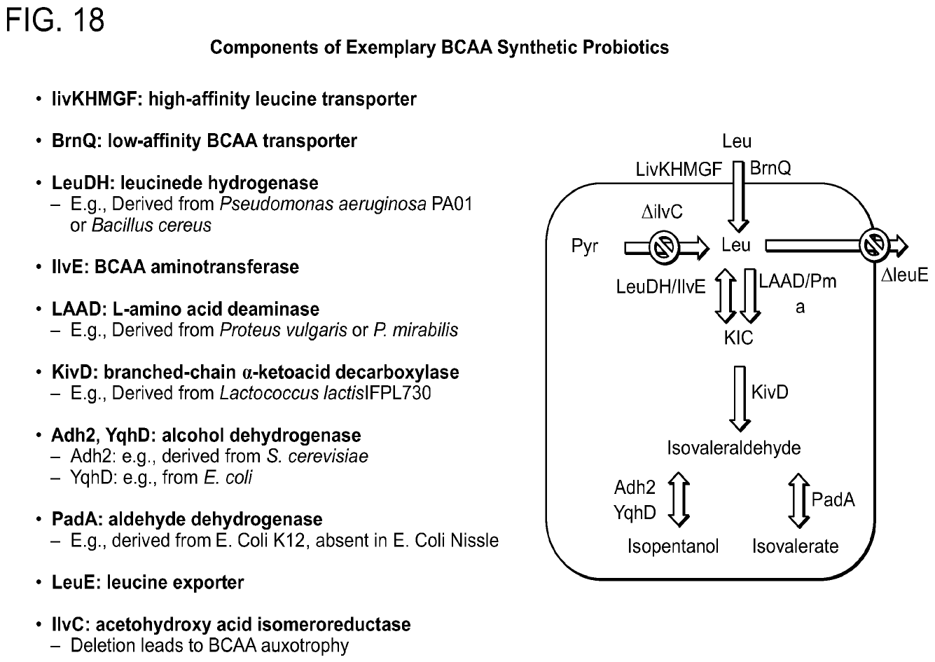

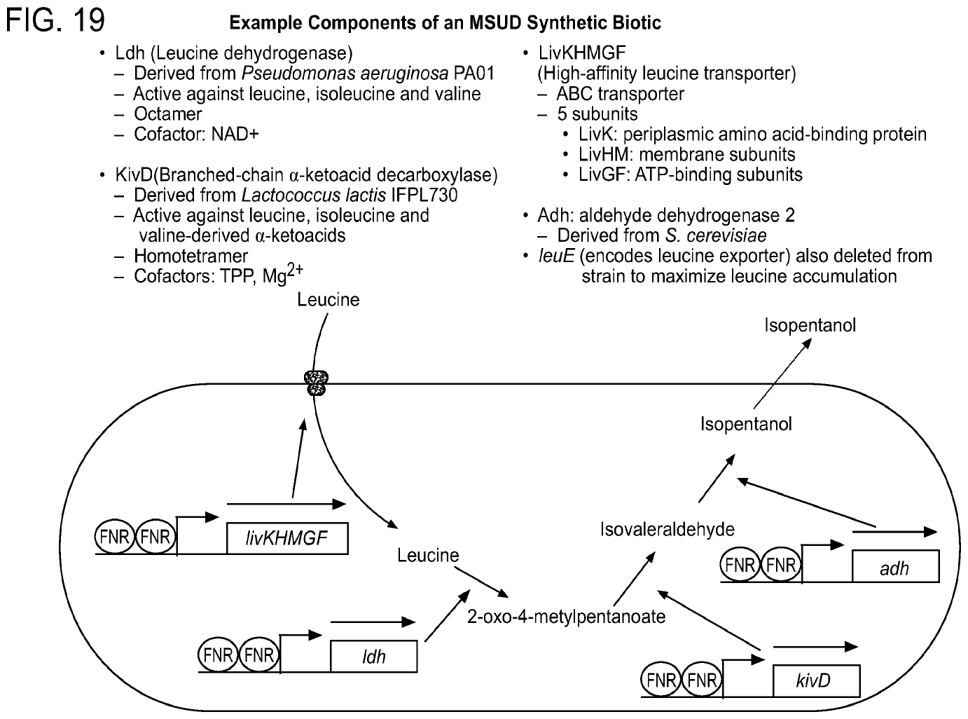

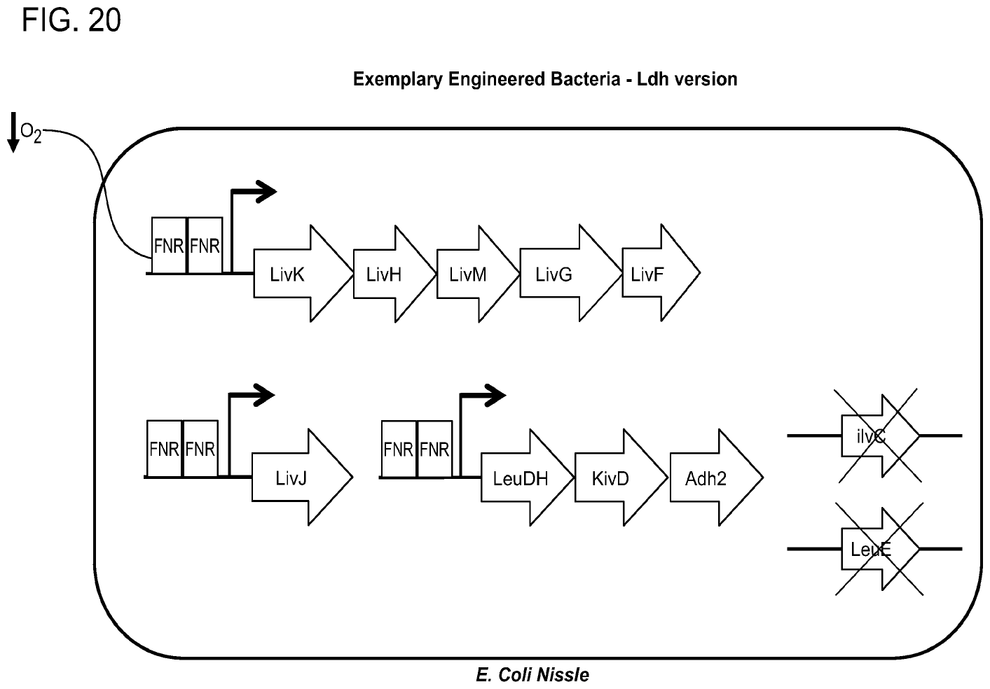

In the above-described kill switch circuits, a toxin is produced in the presence of an environmental factor or signal. In another aspect of kill switch circuitry, a toxin may be repressed in the presence of an environmental factor (not produced) and then produced once the environmental condition or external signal is no longer present. Such kill switches are called repression-based kill switches and represent systems in which the bacterial cells are viable only in the presence of an external factor or signal, such as arabinose or other sugar. Exemplary kill switch designs in which the toxin is repressed in the presence of an external factor or signal (and activated once the external signal is removed) are shown in FIGS. 12-16. The disclosure provides recombinant bacterial cells which express one or more heterologous gene(s) upon sensing arabinose or other sugar in the exogenous environment. In this aspect, the recombinant bacterial cells contain the araC gene, which encodes the AraC transcription factor, as well as one or more genes under the control of the araBAD promoter. In the absence of arabinose, the AraC transcription factor adopts a conformation that represses transcription of genes under the control of the araBAD promoter. In the presence of arabinose, the AraC transcription factor undergoes a conformational change that allows it to bind to and activate the AraBAD promoter, which induces expression of the desired gene, for example tetR, which represses expression of a toxin gene. In this embodiment, the toxing gene is repressed in the presence of arabinose or other sugar. In an environment where arabinose is not present, the tetR gene is not activated and the toxin is expressed, thereby killing the bacteria. The arbinoase system can also be used to express an essential gene, in which the essential gene is only expressed in the presence of arabinose or other sugar and is not expressed when arabinose or other sugar is absent from the environment.

Thus, in some embodiments in which one or more heterologous gene(s) are expressed upon sensing arabinose in the exogenous environment, the one or more heterologous genes are directly or indirectly under the control of the araBAD promoter. In some embodiments, the expressed heterologous gene is selected from one or more of the following: a heterologous therapeutic gene, a heterologous gene encoding an antitoxin, a heterologous gene encoding a repressor protein or polypeptide, for example, a TetR repressor, a heterologous gene encoding an essential protein not found in the bacterial cell, and/or a heterologous encoding a regulatory protein or polypeptide.

Arabinose inducible promoters are known in the art, including Pₐᵣₐ, P_{araB}, P_{araC}, and P_{araBAD}. In one embodiment, the arabinose inducible promoter is from E. coli. In some embodiments, the P_{araC} promoter and the P_{araBAD} promoter operate as a bidirectional promoter, with the P_{araBAD} promoter controlling expression of a heterologous gene(s) in one direction, and the P_{araC} (in close proximity to, and on the opposite strand from the P_{araBAD} promoter), controlling expression of a heterologous gene(s) in the other direction. In the presence of arabinose, transcription of both heterologous genes from both promoters is induced. However, in the absence of arabinose, transcription of both heterologous genes from both promoters is not induced.

In one exemplary embodiment of the disclosure, the genetically engineered bacteria of the present disclosure contains a kill-switch having at least the following sequences: a P_{araBAD} promoter operably linked to a heterologous gene encoding a Tetracycline Repressor Protein (TetR), a P_{araC} promoter operably linked to a heterologous gene encoding AraC transcription factor, and a heterologous gene encoding a bacterial toxin operably linked to a promoter which is repressed by the Tetracycline Repressor Protein (P_{TetR}). In the presence of arabinose, the AraC transcription factor activates the P_{araBAD} promoter, which activates transcription of the TetR protein, which, in turn, represses transcription of the toxin. In the absence of arabinose, however, AraC suppresses transcription from the P_{araBAD} promoter and no TetR protein is expressed. In this case, expression of the heterologous toxin gene is activated, and the toxin is expressed. The toxin builds up in the recombinant bacterial cell, and the recombinant bacterial cell is killed. In one embodiment, the AraC gene encoding the AraC transcription factor is under the control of a constitutive promoter and is therefore constitutively expressed.

In one embodiment of the disclosure, the genetically engineered bacterium further comprises an antitoxin under the control of a constitutive promoter. In this situation, in the presence of arabinose, the toxin is not expressed due to repression by TetR protein, and the antitoxin protein builds-up in the cell. However, in the absence of arabinose, TetR protein is not expressed, and expression of the toxin is induced. The toxin begins to build-up within the recombinant bacterial cell. The recombinant bacterial cell is no longer viable once the toxin protein is present at either equal or greater amounts than that of the anti-toxin protein in the cell, and the recombinant bacterial cell will be killed by the toxin.

In another embodiment of the disclosure, the genetically engineered bacterium further comprises an antitoxin under the control of the P_{araBAD} promoter. In this situation, in the presence of arabinose, TetR and the anti-toxin are expressed, the anti-toxin builds up in the cell, and the toxin is not expressed due to repression by TetR protein. However, in the absence of arabinose, both the TetR protein and the anti-toxin are not expressed, and expression of the toxin is induced. The toxin begins to build-up within the recombinant bacterial cell. The recombinant bacterial cell is no longer viable once the toxin protein is expressed, and the recombinant bacterial cell will be killed by the toxin.

In another exemplary embodiment of the disclosure, the genetically engineered bacteria of the present disclosure contain a kill-switch having at least the following sequences: a P_{araBAD} promoter operably linked to a heterologous gene encoding an essential polypeptide not found in the recombinant bacterial cell (and required for survival), and a P_{araC} promoter operably linked to a heterologous gene encoding AraC transcription factor. In the presence of arabinose, the AraC transcription factor activates the P_{araBAD} promoter, which activates transcription of the heterologous gene encoding the essential polypeptide, allowing the recombinant bacterial cell to survive. In the absence of arabinose, however, AraC suppresses transcription from the P_{araBAD} promoter and the essential protein required for survival is not expressed. In this case, the recombinant bacterial cell dies in the absence of arabinose. In some embodiments, the sequence of P_{araBAD} promoter operably linked to a heterologous gene encoding an essential polypeptide not found in the recombinant bacterial cell can be present in the bacterial cell in conjunction with the TetR/toxin kill-switch system described directly above. In some embodiments, the sequence of P_{araBAD} promoter operably linked to a heterologous gene encoding an essential polypeptide not found in the recombinant bacterial cell can be present in the bacterial cell in conjunction with the TetR/toxin/anto-toxin kill-switch system described directly above.

In yet other embodiments, the bacteria may comprise a plasmid stability system with a plasmid that produces both a short-lived anti-toxin and a long-lived toxin. In this system, the bacterial cell produces equal amounts of toxin and anti-toxin to neutralize the toxin. However, if/when the cell loses the plasmid, the short-lived anti-toxin begins to decay. When the anti-toxin decays completely the cell dies as a result of the longer-lived toxin killing it.

In some embodiments, the engineered bacteria of the present disclosure that are capable of producing a substrate transporter further comprise the gene(s) encoding the components of any of the above-described kill switch circuits.

In any of the above-described embodiments, the bacterial toxin is selected from the group consisting of a lysin, Hok, Fst, TisB, LdrD, Kid, SymE, MazF, FlmA, Ibs, XCV2162, dinJ, CcdB, MazF, ParE, YafO, Zeta, hicB, relB, yhaV, yocB, chpBK, hipA, microcin B, microcin B17, microcin C, microcin C7-C51, microcin J25, microcin ColV, microcin 24, microcin L, microcin D93, microcin L, microcin E492, microcin H47, microcin I47, microcin M, colicin A, colicin E1, colicin K, colicin N, colicin U, colicin B, colicin Ia, colicin Ib, colicin 5, colicin 10, colicin S4, colicin Y, colicin E2, colicin E7, colicin E8, colicin E9, colicin E3, colicin E4, colicin E6; colicin E5, colicin D, colicin M, and cloacin DF13, or a biologically active fragment thereof.

In any of the above-described embodiments, the anti-toxin is selected from the group consisting of an anti-lysin, Sok, RNAII, IstR, RdID, Kis, SymR, MazE, FlmB, Sib, ptaRNA1, yafQ, CcdA, MazE, ParD, yafN, Epsilon, HicA, relE, prIF, yefM, chpBI, hipB, MccE, MccE^{CTD}, MccF, Cai, ImmEl, Cki, Cni, Cui, Chi, lia, Imm, Cfi, Im10, Csi, Cyi, Im2, Im7, Im8, Im9, Im3, Im4, ImmE6, cloacin immunity protein (Cim), ImmE5, ImmD, and Cmi, or a biologically active fragment thereof.

In one embodiment, the bacterial toxin is bactericidal to the genetically engineered bacterium. In one embodiment, the bacterial toxin is bacteriostatic to the genetically engineered bacterium.

In some embodiments, provided herein are genetically engineered bacteria comprising a heterologous gene encoding a substrate transporter, wherein the gene or gene cassette for producing the substrate transporter is controlled by a promoter that is induced under low-oxygen or anaerobic conditions. In some embodiments, the promoter is selected from the fumarate and nitrate reductase regulator (FNR) promoter, arginine deiminiase and nitrate reduction (ANR) promoter, and dissimilatory nitrate respiration regulator (DNR) promoter.

In some embodiments, the genetically engineered bacteria comprising a heterologous gene encoding a substrate transporter is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyA and ΔdapA auxotroph.

In some embodiments, the genetically engineered bacteria comprising a heterologous gene encoding a substrate transporter further comprises a kill switch circuit, such as any of the kill switch circuits provided herein. For example, in some embodiments, the genetically engineered bacteria further comprise one or more genes encoding one or more recombinase(s) under the control of an inducible promoter and an inverted toxin sequence. In some embodiments, the genetically engineered bacteria further comprise one or more genes encoding an anti-toxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an anti-toxin.

In some instances, basal or leaky expression from an inducible promoter may result in the activation of the kill switch, thereby creating strong selective pressure for one or more mutations that disable the switch and thus the ability to kill the cell. In some embodiments, an environmental factor, e.g. arabinose, is present during manufacturing, and activates the production of a repressor that shuts down toxin production. Mutations in this circuit, with the exception of the toxin gene itself, will result in death with reduced chance for negative selection. When the environmental factor is absent, the repressor stops being made, and the toxin is produced. When the toxin concentration overcomes that of the antitoxin, the cell dies. In some embodiments, variations in the promoter and ribosome binding sequences of the antitoxin and the toxin allow for tuning of the circuit to produce variations in the timing of cell death. In alternate embodiments, the circuit comprises recombinases that are repressed by tetR and produced in the absence of tetR. These recombinases are capable of flipping the toxin gene or its promoter into the active configuration, thereby resulting in toxin production.

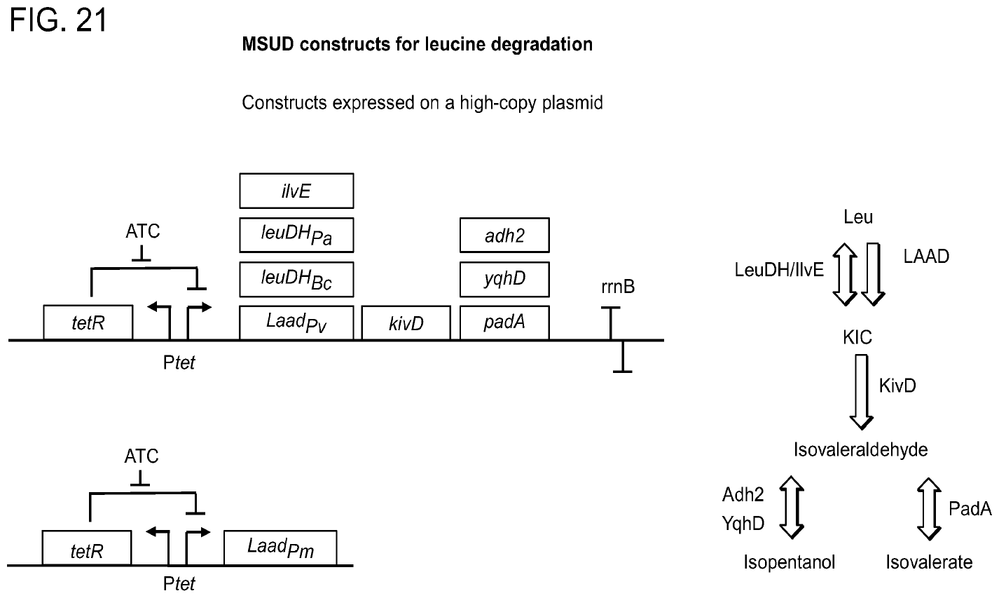

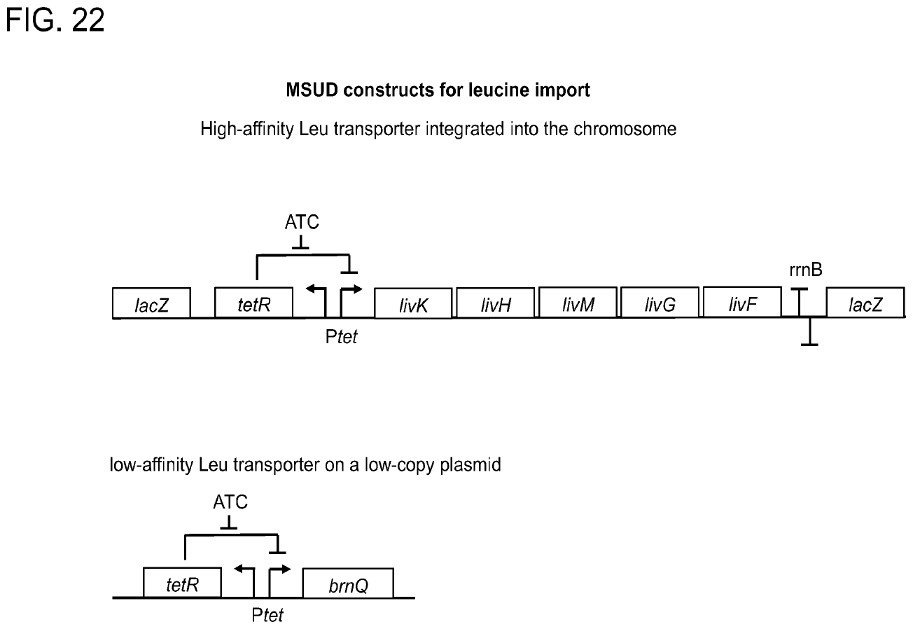

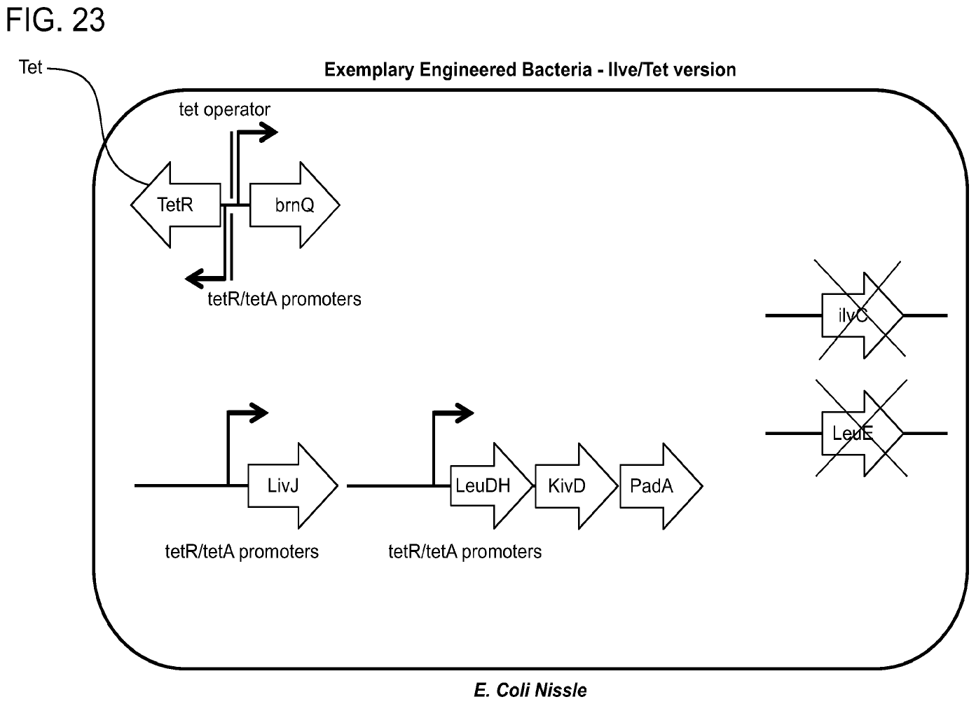

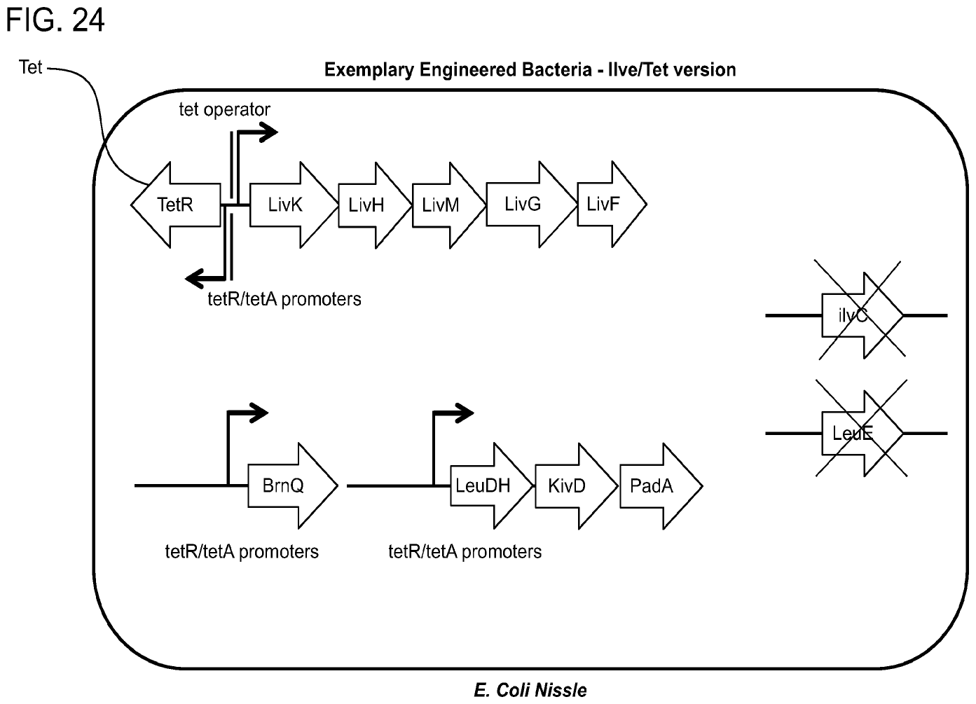

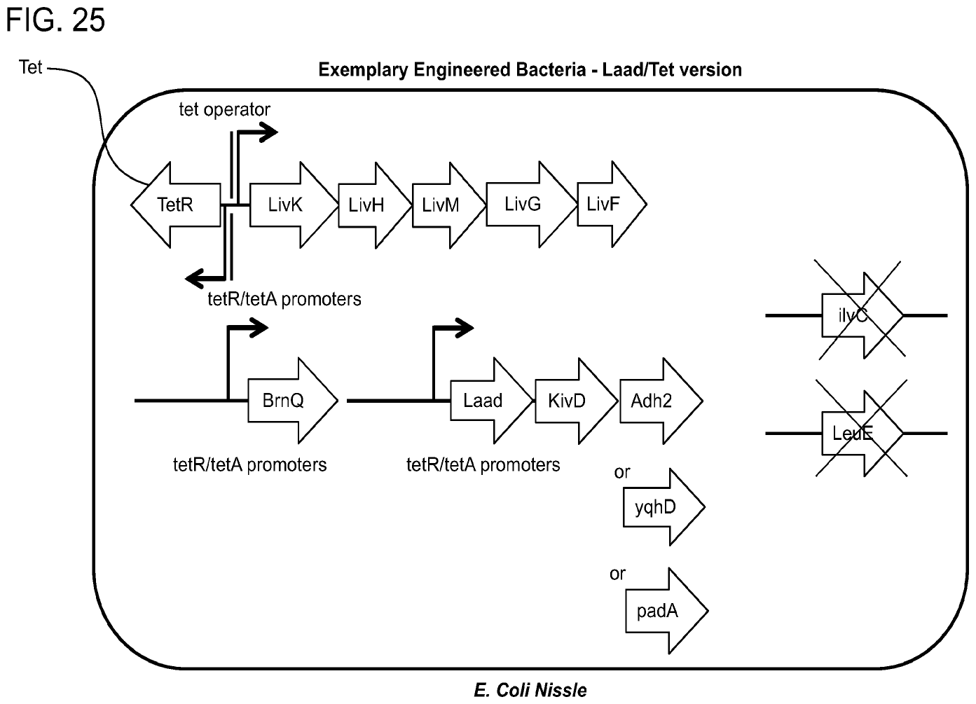

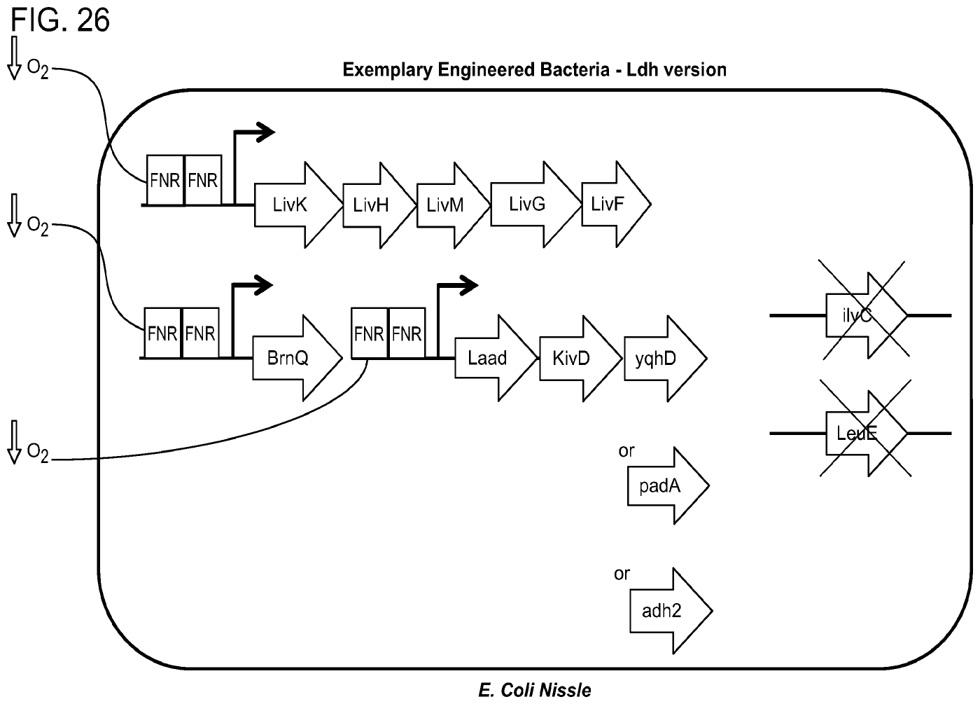

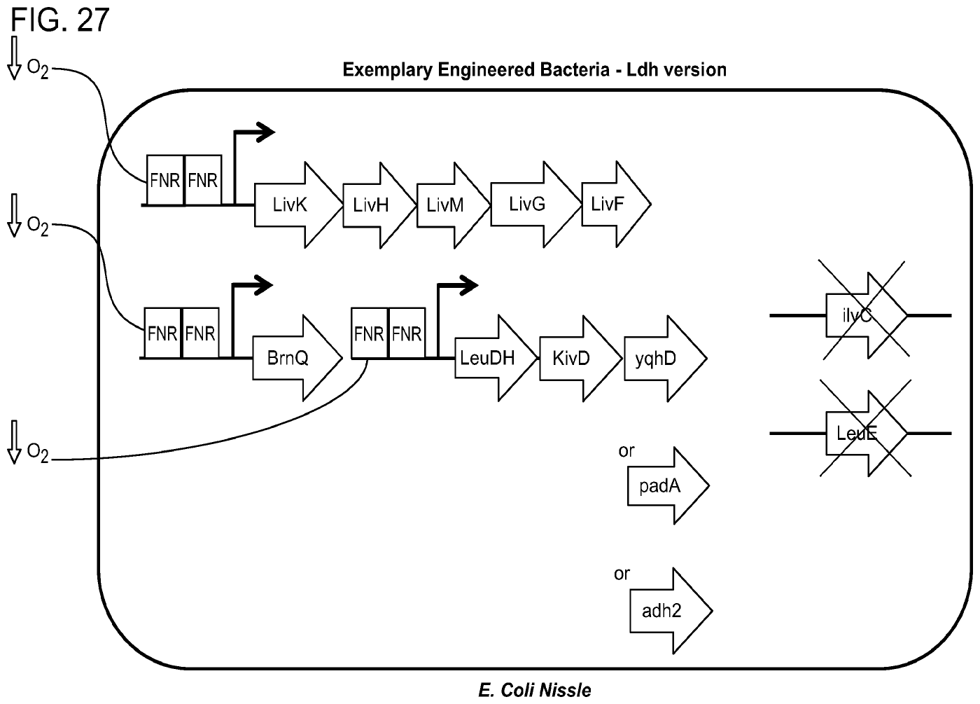

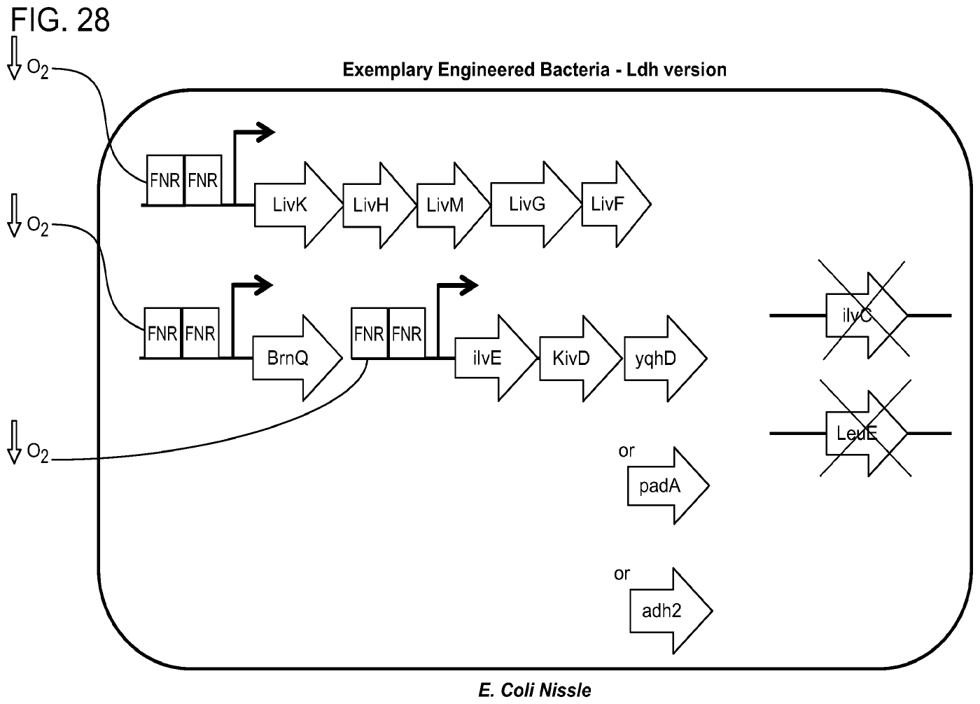

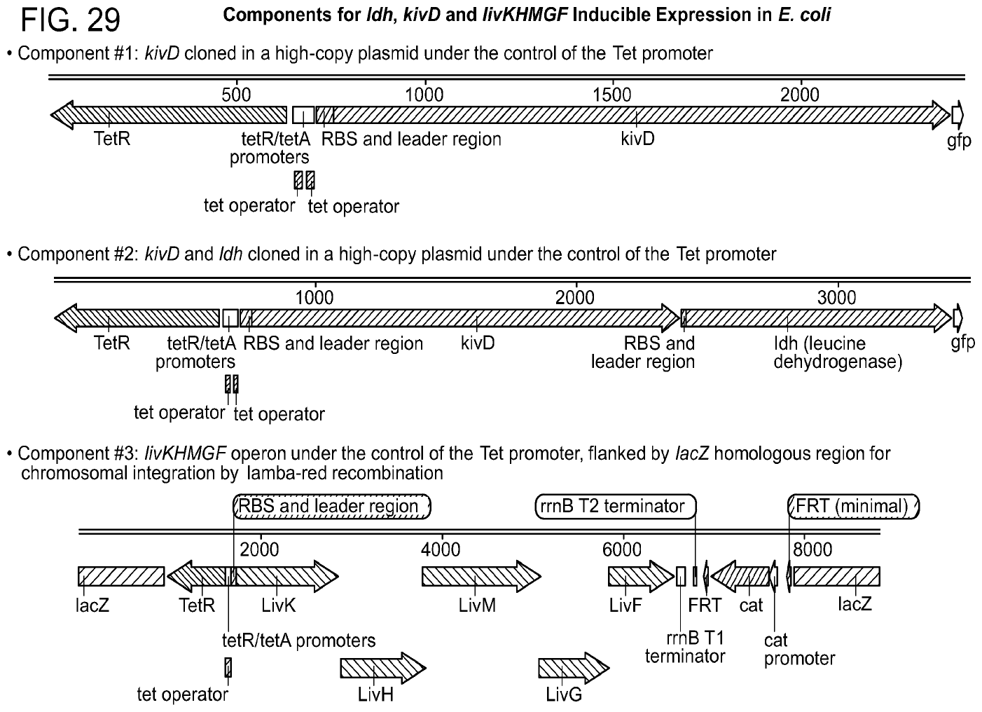

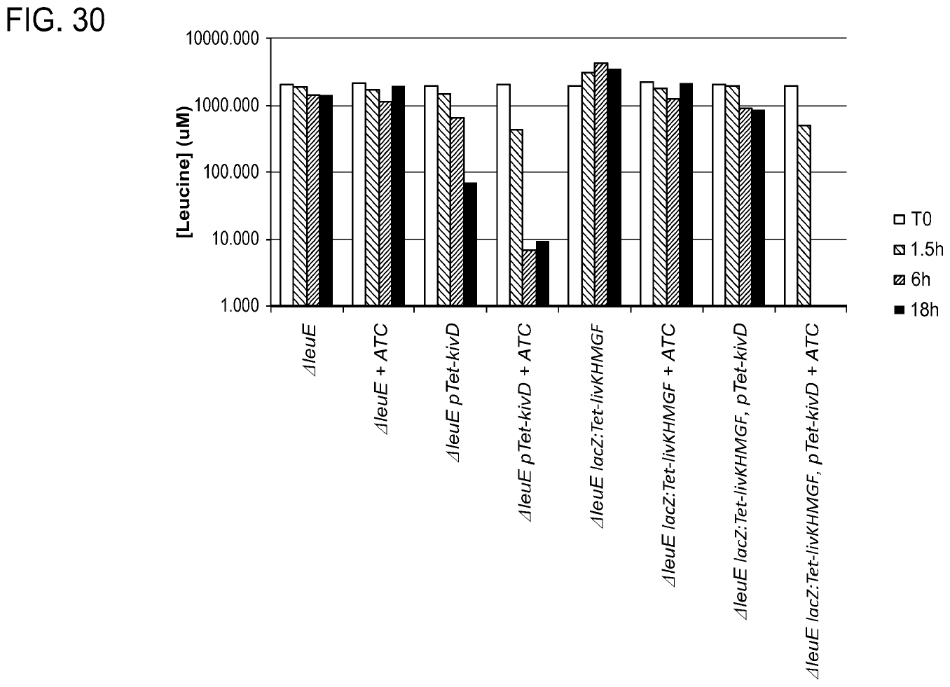

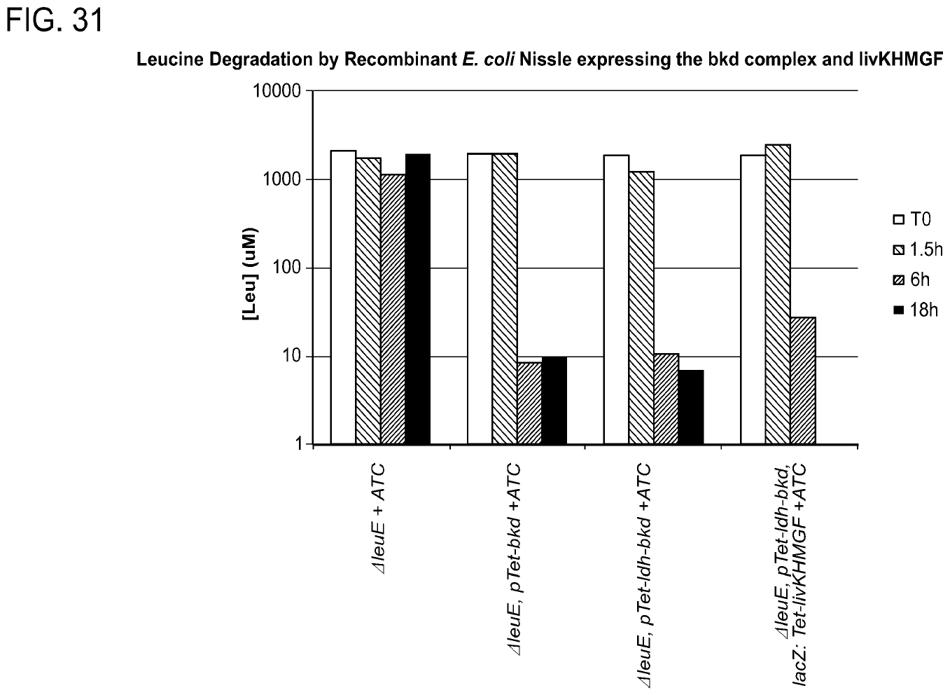

Synthetic gene circuits express on plasmids may function well in the short term but lose ability and/or function in the long term, e.g., in the stringent conditions found in a tumor microenvironment (Danino et al. (2015) Sci. Transl. Med. 7 (289): 289ra84). In some embodiments, the genetically engineered bacteria comprise stable circuits for expressing genes of interest, e.g., a substrate transporter, over prolonged periods. In some embodiments, the genetically engineered bacteria are capable of targeting cancerous cells and producing a substrate transporter and further comprise a toxin-antitoxin system that simultaneously produces a toxin (hok) and a short-lived antitoxin (sok), wherein loss of the plasmid causes the cell to be killed by the long-lived toxin (Danino et al., 2015; FIG. 21). In some embodiments, the genetically engineered bacteria further comprise alp7 from B. subtilis plasmid pL20 and produces filaments that are capable of pushing plasmids to the poles of the cells in order to ensure equal segregation during cell division (Danino et al., 2015).

In some embodiments, the genetically engineered bacteria comprising a heterologous gene encoding a substrate transporter is an auxotroph and further comprises a kill switch circuit, such as any of the kill switch circuits described herein.

In some embodiments of the above described genetically engineered bacteria, the gene encoding the substrate transporter is present on a plasmid in the bacterium and operatively linked on the plasmid to the promoter that is induced under low-oxygen or anaerobic conditions. The genetically engineered bacteria are capable of local and tumor-specific delivery of the substrate transporter, e.g., an amino acid transporter. In other embodiments, the gene encoding the substrate transporter is present in the bacterial chromosome and is operatively linked in the chromosome to the promoter that is induced under low-oxygen or anaerobic conditions. The genetically engineered bacteria are capable of local and tumor-specific delivery of the substrate transporter.

### Pharmaceutical Compositions and Formulations

Pharmaceutical compositions comprising the genetically engineered microrganisms of the invention may be used to treat, manage, ameliorate, and/or prevent a disease or condition disclosed herein. Pharmaceutical compositions of the invention comprising one or more genetically engineered bacteria with prophylactic agents, therapeutic agents, and/or pharmaceutically acceptable carriers are provided.

In certain embodiments, the pharmaceutical composition comprises one species, strain, or subtype of bacteria that are engineered to comprise the genetic modifications described herein, e.g., one or more genes encoding one or more substrate transporters. In alternate embodiments, the pharmaceutical composition comprises two or more species, strains, and/or subtypes of bacteria that are each engineered to comprise the genetic modifications described herein, e.g., one or more genes encoding one or more substrate transporters.

In some embodiments, the genetically engineered bacteria are administered systemically or intratumorally as spores. As a non-limiting example, the genetically engineered bacteria are Clostridia, and administration results in a selective colonization of hypoxic/necrotic areas within a tumor. In some embodiments, the spores germinate exclusively in the hypoxic/necrotic regions present in solid tumours and nowhere else in the body.

The pharmaceutical compositions of the invention may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, PA). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intratumoral, peritumor, immediate-release, pulsatile-release, delayed-release, or sustained release). Suitable dosage amounts for the genetically engineered bacteria may range from about 10⁴ to 10¹² bacteria. The composition may be administered once or more daily, weekly, or monthly. The composition may be administered before, during, or following a meal. In one embodiment, the pharmaceutical composition is administered before the subject cats a meal. In one embodiment, the pharmaceutical composition is administered currently with a meal. In on embodiment, the pharmaceutical composition is administered after the subject cats a meal.

The genetically engineered bacteria or genetically engineered virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection. Alternatively, the genetically engineered microorganisms may be administered intratumorally and/or peritumorally. In other embodiments, the genetically engineered microorganisms may be administered intra-arterially, intramuscularly, or intraperitoneally. In some embodiments, the genetically engineered bacteria colonize about 20%, 30%, 40%, 50%, 60%, 70%, 80%, 90% or more of the target site (e.g., a tumor). In some embodiments, the genetically engineered bacteria are co-administered with a PEGylated form of rHuPH20 (PEGPH20) or other agent in order to destroy the tumor septae in order to enhance penetration of the tumor capsule, collagen, and/or stroma. In some embodiments, the genetically engineered bacteria are capable of producing a substrate transporter as well as one or more enzymes that degrade fibrous tissue.

The genetically engineered microroganisms of the disclosure may be administered via intratumoral injection, resulting in bacterial cells that are directly deposited within the target tissue (e.g., a tumor). Intratumoral injection of the engineered bacteria may elicit a potent localized inflammatory response as well as an adaptive immune response against tumor cells. Bacteria are suspended in solution before being withdrawn into a 1-ml syringe. In some embodiments, the tumor is injected with an 18-gauge multipronged needle (Quadra-Fuse, Rex Medical). The injection site is aseptically prepared. If available, ultrasound or CT may be used to identify a necrotic region of the tumor for injection. If a necrotic region is not identified, the injection can be directed to the center of the tumor. The needle is inserted once into a predefined region, and dispensed with even pressure. The injection needle is removed slowly, and the injection site is sterilized.

Direct intratumoral injection of the genetically engineered bacteria of the invention into a target tissue (e.g., a solid tumor) may be advantageous as compared to intravenous administration. Using an intravenous injection method, only a small proporation of the bacteria may reach the target tumor. For example, following E. coli Nissle injection into the tail vein of 4T1 tumor-bearing mice, most bacteria (>99%) are quickly cleared from the animals and only a small percentage of the administered bacteria colonize the tumor (Stritzker et al., 2007). In particular, in large animals and human patients, which have relatively large blood volumes and relatively small tumors compared to mice, intratumoral injection may be especially beneficial. Injection directly into the tumor allows the delivery of a higher concentration of therapeutic agent and avoids the toxicity, which can result from systemic administration. In addition, intratumoral injection of bacteria induces robust and localized immune responses within the tumor.

Depending on the location, tumor type, and tumor size, different administration techniques may be used, including but not limited to, cutaneous, subcutaneous, and percutaneous injection, therapeutic endoscopic ultrasonography, or endobronchial intratumor delivery. Prior to the intratumor administration procedures, sedation in combination with a local anesthetic and standard cardiac, pressure, and oxygen monitoring, or full anesthesia of the patient is performed.

For some tumors, percutaneous injection can be employed, which is the least invasive administration method. Ultrasound, computed tomography (CT) or fluoroscopy can be used as guidance to introduce and position the needle. Percutaneous intratumoral injection is for example described for hepatocellular carcinoma in Lencioni et al. (2010) J. Vasc Interv Radiol. 21 (10): 1533-8). Intratumoral injection of cutaneous, subcutaneous, and nodal tumors is for example described in WO/2014/036412 (Amgen) for late stage melanoma.

Single insertion points or multiple insertion points can be used in percutaneous injection protocols. Using a single insertion point, the solution may be injected percutaneously along multiple tracks, as far as the radial reach of the needle allows. In other embodiments, multiple injection points may be used if the tumor is larger than the radial reach of the needle. The needle can be pulled back without exiting, and redirected as often as necessary until the full dose is injected and dispersed. To maintain sterility, a separate needle is used for each injection. Needle size and length varies depending on the tumor type and size.

In some embodiments, the tumor is injected percutaneously with an 18-gauge multipronged needle (Quadra-Fuse, Rex Medical). The device consists of an 18 gauge puncture needle 20 cm in length. The needle has three retractable prongs, each with four terminal side holes and a connector with extension tubing clamp. The prongs are deployed from the lateral wall of the needle. The needle can be introduced percutaneously into the center of the tumor and can be positioned at the deepest margin of the tumor. The prongs are deployed to the margins of the tumor. The prongs are deployed at maximum length and then are retracted at defined intervals. Optionally, one or more rotation-injection-rotation maneuvers can be performed, in which the prongs are retracted, the needle is rotated by a 60 degrees, which is followed by repeat deployment of the prongs and additional injection.

Therapeutic endoscopic ultrasonography (EUS) is employed to overcome the anatomical constraints inherent in gaining access to certain other tumors (Shirley et al. (2013) Gastroenterol Res. Pract. 2013:207129). EUS-guided fine needle injection (EUS-FNI) has been successfully used for antitumor therapies for the treatment of head and neck, esophageal, pancreatic, hepatic, and adrenal masses (Verna et al. (2008) Therap. Adv Gastroenterol. 1 (2): 103-9). EUS-FNI has been extensively used for pancreatic cancer injections. Fine-needle injection requires the use of the curvilinear echoendoscope. The esophagus is carefully intubated and the echoendoscope is passed into the stomach and duodenum where the pancreatic examination occurs and the target tumor is identified. The largest plane is measured to estimate the tumor volume and to calculate the injection volume. The appropriate volume is drawn into a syringe. A primed 22-gauge fine needle aspiration (FNA) needle is passed into the working channel of the echoendoscope. Under ultrasound guidance, the needle is passed into the tumor. Depending on the size of the tumor, administration can be performed by dividing the tumor into sections and then injecting the corresponding fractions of the volume into each section. Use of an installed endoscopic ultrasound processor with Doppler technology assures there are no arterial or venous structures that may interfere with the needle passage into the tumor (Shirley et al., 2013). In some embodiments, ‘multiple injectable needle’ (MIN) for EUS-FNI can be used to improvement the injection distribution to the tumor in comparison with straight-type needles (Ohara et al. (2013) Mol. Clin. Oncol. 1 (2): 231-4).

Intratumoral administration for lung cancer, such as non-small cell lung cancer, can be achieved through endobronchial intratumor delivery methods, as described in Celikoglu et al., 2008. Bronchoscopy (trans-nasal or oral) is conducted to visualize the lesion to be treated. The tumor volume can be estimated visually from visible length-width height measurements over the bronchial surface. The needle device is then introduced through the working channel of the bronchoscope. The needle catheter, which consists of a metallic needle attached to a plastic catheter, is placed within a sheath to prevent damage by the needle to the working channel during advancement. The needle size and length varies and is determined according to tumor type and size of the tumor. Needles made from plastic are less rigid than metal needles and are ideal, since they can be passed around sharper bends in the working channel. The needle is inserted into the lesion and the genetically engineered bacteria of the invention are in injected. Needles are inserted repeatedly at several insertion points until the tumor mass is completely perfused. After each injection, the needle is withdrawn entirely from the tumor and is then embedded at another location. At the end of the bronchoscopic injection session, removal of any necrotic debris caused by the treatment may be removed using mechanical dissection, or other ablation techniques accompanied by irrigation and aspiration.

In some embodiments, the genetically engineered bacteria are administrated directly into the tumor using methods, including but not limited to, percutaneous injection, EUS-FNI, or endobronchial intratumor delivery methods. In some cases other techniques, such as laproscopic or open surgical techniques are used to access the target tumor, however, these techniques are much more invasive and bring with them much greater morbidity and longer hospital stays.

In some embodiments, bacteria, e.g., E. coli Nissle, or spores, e.g., Clostridium novyi NT, are dissolved in sterile phosphate buffered saline (PBS) for systemic or intratumor injection.

The dose to be injected is derived from the type and size of the tumor. The dose of a drug or the genetically engineered bacteria or virus of the invention is typically lower, e.g., orders of magniture lower, than a dose for systemic intravenous administration.

The volume injected into each lesion is based on the size of the tumor. To obtain the tumor volume, a measurement of the largest plane can be conducted. The estimated tumor volume can then inform the determination of the injection volume as a percentage of the total volume. For example, an injection volume of approximately 20-40% of the total tumor volume can be used.

For example, as is described, for example, in WO 2014/036412, for tumors larger than 5 cm in their largest dimension, up to 4 ml can be injected. For tumors between 2.5 and 5 cm in their largest dimension, up to 2 ml can be injected. For tumors between 2.5 and 5 cm in their largest dimension, up to 2 ml can be injected. For tumors between 1.5 and 2.5 cm in their largest dimension, up to 1 ml can be injected. For tumors between 0.5 and 1.5 cm in their largest dimension, up to 0.5 ml can be injected. For tumors equal or small than 0.5 in their largest dimension, up to 0.1 ml can be injected. Alternatively, ultrasound scan can be used to determine the injection volume that can be taken up by the tumor without leakage into surrounding tissue.

In some embodiments, the treatment regimen will include one or more intratumoral administrations. In some embodiments, a treatment regimen will include an initial dose, which followed by at least one subsequent dose. One or more doses can be administered sequentially in two or more cycles.

For example a first dose may be administered at day 1, and a second dose may be administered after 1, 2, 3, 4, 5, 6, days or 1, 2, 3, or 4 weeks or after a longer interval. Additional doses may be administered after 1, 2, 3, 4, 5, 6, days or after 1, 2, 3, or 4 weeks or longer intervals. In some embodiments, the first and subsequent administrations have the same dosage. In other embodiments, different doses are administered. In some embodiments, more than one dose is administered per day, for example, two, three or more doses can be administered per day.

The routes of administration and dosages described are intended only as a guide. The optimum route of administration and dosage can be readily determined by a skilled practitioner. The dosage may be determined according to various parameters, especially according to the location of the tumor, the size of the tumor, the age, weight and condition of the patient to be treated and the route and method of administration.

In one embodiment, Clostridium spores are delivered systemically. In another embodiment, Clostridium spores are delivered via intratumor injection. In one embodiment, E. coli Nissle are delivered via intratumor injection In other embodiments, E. coli Nissle, which is known to hone to tumors, is administered via intravenous injection or orally, as described in a mouse model in for example in Danino et al. 2015, or Stritzker et al., 2007, the contents of which is herein incorporated by reference in its entirety. E. coli Nissle mutations to reduce toxicity include but are not limited to msbB mutants resulting in non-myristoylated LPS and reduced endotoxin activity, as described in Stritzker et al., 2010 (Stritzker et al, Bioengineered Bugs 1:2, 139-145; Myroystoation negative msbB-mutants of probiotic E. coli Nissle 1917 retain tumor specific colonization properties but show less side effects in immunocompetent mice.

For intravenous injection a preferred dose of bacteria is the dose in which the greatest number of bacteria is found in the tumor and the lowest amount found in other tissues. In mice, Stritzker et al. (2007) Int. J. Med. Microbiol. 297 (2007) 151-162) found that the lowest number of bacteria needed for successful tumor colonization was 2×10⁴ CFU, in which half of the mice showed tumor colonization. Injection of 2×10⁵ and 2×10⁶ CFU resulted in colonization of all tumors, and numbers of bacteria in the tumors increased. However, at higher concentrations, bacterial counts became detectable in the liver and the spleen.

In some embodiments, the genetically engineered microorganisms of the invention may be administered orally. In some embodiments the genetically engineered bacteria may be useful in the prevention, treatment or management of liver cancer or liver metastases. For example, Danino et al. showed that orally administered E. coli Nissle is able to colonize liver metastases by crossing the gastrointestinal tract in a mouse model of liver metastases (Danino et al., Science Translational Medicine 7 (289): 1-10, the contents of which is herein incorporated by reference in its entirety).

Tumor types into which the engineered bacteria of the current invention are intratumorally delivered include locally advanced and metastatic tumors, including but not limited to, B, T, and NK cell lymphomas, colon and rectal cancers, melanoma, including metastatic melanoma, mycosis fungoides, Merkel carcinoma, liver cancer, including hepatocellular carcinoma and liver metastasis secondary to colorectal cancer, pancreatic cancer, breast cancer, follicular lymphoma, prostate cancer, refractory liver cancer, and Merkel cell carcinoma.

The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, PA. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliccous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134 (2): 361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intrailcal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD₅₀, ED₅₀, EC₅₀, and IC₅₀ may be determined, and the dose ratio between toxic and therapeutic effects (LD₅₀/ED₅₀) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically scaled container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically scaled container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered microorganisms and composition thereof is formulated for intravenous administration, intratumor administration, or peritumor administration. The genetically engineered microorganisms may be formulated as depot preparations. Such long acting formulations may be administered by implantation or by injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (scc e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylenc glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Methods of Treatment

In one aspect of the invention provides methods of treating a disease, disorder and/or a symptom of a disease or disorder described herein. In one aspect of the invention provides methods of treating cancer. In some embodiments, the invention provides methods for reducing, ameliorating, or eliminating one or more symptom(s) associated with cancer. In some embodiments, the cancer is selected from adrenal cancer, adrenocortical carcinoma, anal cancer, appendix cancer, bile duct cancer, bladder cancer, bone cancer (e.g., Ewing sarcoma tumors, osteosarcoma, malignant fibrous histiocytoma), brain cancer (e.g., astrocytomas, brain stem glioma, craniopharyngioma, ependymoma), bronchial tumors, central nervous system tumors, breast cancer, Castleman disease, cervical cancer, colon cancer, rectal cancer, colorectal cancer, endometrial cancer, esophageal cancer, eye cancer, gallbladder cancer, gastrointestinal cancer, gastrointestinal carcinoid tumors, gastrointestinal stromal tumors, gestational trophoblastic disease, heart cancer, Kaposi sarcoma, kidney cancer, largyngeal cancer, hypopharyngeal cancer, leukemia (e.g., acute lymphoblastic leukemia, acute myeloid leukemia, chronic lymphocytic leukemia, chronic myelogenous leukemia), liver cancer, lung cancer, lymphoma (e.g., AIDS-related lymphoma, Burkitt lymphoma, cutaneous T cell lymphoma, Hogkin lymphoma, Non-Hogkin lymphoma, primary central nervous system lymphoma), malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumors, prostate cancer, retinoblastoma, rhabdomyosarcoma, rhabdoid tumor, salivary gland cancer, sarcoma, skin cancer (e.g., basal cell carcinoma, melanoma), small intestine cancer, stomach cancer, teratoid tumor, testicular cancer, throat cancer, thymus cancer, thyroid cancer, unusual childhood cancers, urethral cancer, uterine cancer, uterine sarcoma, vaginal cancer, vulvar cancer, Waldenström macrogloblulinemia, and Wilms tumor. In some embodiments, the symptom(s) associated thereof include, but are not limited to, anemia, loss of appetite, irritation of bladder lining, bleeding and bruising (thrombocytopenia), changes in taste or smell, constipation, diarrhea, dry mouth, dysphagia, edema, fatigue, hair loss (alopecia), infection, infertility, lymphedema, mouth sores, nausea, pain, peripheral neuropathy, tooth decay, urinary tract infections, and/or problems with memory and concentration.

The method may comprise preparing a pharmaceutical composition with at least one genetically engineered species, strain, or subtype of bacteria described herein, and administering the pharmaceutical composition to a subject in a therapeutically effective amount. The genetically engineered microorganisms may be administered locally, e.g., intratumorally or peritumorally into a tissue or supplying vessel, or systemically, e.g., intravenously by infusion or injection. In some embodiments, the genetically engineered bacteria are administered intravenously, intratumorally, intra-arterially, intramuscularly, intraperitoneally, orally, or topically. In some embodiments, the genetically engineered microorganisms are administered intravenously, i.e., systemically.

In certain embodiments, administering the pharmaceutical composition to the subject reduces cell proliferation, tumor growth, and/or tumor volume in a subject. In some embodiments, the methods of the present disclosure may reduce cell proliferation, tumor growth, and/or tumor volume by at least about 10%, 20%, 25%, 30%, 40%, 50%, 60%, 70%, 75%, 80%, 85%, 90%, 95%, or more as compared to levels in an untreated or control subject. In some embodiments, reduction is measured by comparing cell proliferation, tumor growth, and/or tumor volume in a subject before and after administration of the pharmaceutical composition. In some embodiments, the method of treating or ameliorating a cancer in a subject allows one or more symptoms of the cancer to improve by at least about 10%, 20%, 30%, 40%, 50%, 60%, 70%, 80%, 90%, 95%, or more.

Before, during, and after the administration of the pharmaceutical composition, cancerous cells and/or biomarkers in a subject may be measured in a biological sample, such as blood, serum, plasma, urine, peritoneal fluid, and/or a biopsy from a tissue or organ. In some embodiments, the methods may include administration of the compositions of the invention to reduce tumor volume in a subject to an undetectable size, or to less than about 1%, 2%, 5%, 10%, 20%, 25%, 30%, 40%, 50%, 60%, 70%, 75%, 80%, or 90% of the subject's tumor volume prior to treatment. In other embodiments, the methods may include administration of the compositions of the invention to reduce the cell proliferation rate or tumor growth rate in a subject to an undetectable rate, or to less than about 1%, 2%, 5%, 10%, 20%, 25%, 30%, 40%, 50%, 60%, 70%, 75%, 80%, or 90% of the rate prior to treatment.

The genetically engineered bacteria may be destroyed, e.g., by defense factors in tissues or blood serum (Sonnenborn et al. (2009) Microbial Ecology in Health and Disease 21:122-58), or by activation of a kill switch, several hours or days after administration. Thus, the pharmaceutical composition comprising the genetically engineered bacteria comprising a heterologous gene encoding a substrate transporter may be re-administered at a therapeutically effective dose and frequency. In alternate embodiments, the genetically engineered bacteria are not destroyed within hours or days after administration and may propagate and colonize the tumor.

The pharmaceutical composition may be administered alone or in combination with one or more additional therapeutic agents, e.g., a chemotherapeutic drug such a methotrexate. An important consideration in selecting the one or more additional therapeutic agents is that the agent(s) should be compatible with the genetically engineered bacteria of the invention, e.g., the agent(s) must not kill the bacteria. In some studies, the efficacy of anticancer immunotherapy, e.g., CTLA-4 or PD-1 inhibitors, requires the presence of particular bacterial strains in the microbiome (Ilda et al., 2013; Vetizou et al., 2015; Sivan et al., 2015). In some embodiments, the pharmaceutical composition is administered with one or more commensal or probiotic bacteria, e.g., Bifidobacterium or Bacteroides.

In some embodiments, the genetically engineered microorganisms may be administered as part of a regimen, which includes other treatment modalities or combinations of other modalities. Non-limiting examples of these modalities or agents are conventional therapies (e.g., radiotherapy, chemotherapy), other immunotherapies, stem cell therapies, and targeted therapies, (e.g., BRAF or vascular endothelial growth factor inhibitors; antibodies or compounds), bacteria described herein, and oncolytic viruses. Therapies also include related to antibody-immune engagement, including Fc-mediated ADCC therapies, therapies using bispecific soluble scFvs linking cytotoxic T cells to tumor cells (e.g., BiTE), and soluble TCRs with effector functions. Immunotherapies include vaccines (e.g., viral antigen, tumor associated antigen, neoantigen, or combinations thereof), checkpoint inhibitors, cytokine therapies, adoptive cellular therapy (ACT). ACT includes but is not limited to, tumor infiltrating lymphocyte (TIL) therapies, native or engineered TCR or CAR-T therapies, natural killer cell therapies, and dendritic cell vaccines or other vaccines of other antigen presenting cells. Targeted therapies include antibodies and chemical compounds, and include for example antiangiogenic strategies and BRAF inhibition.

The immunostimulatory activity of bacterial DNA is mimicked by synthetic oligodeoxynucleotides (ODNs) expressing unmethylated CpG motifs (see, e.g., Bode et al. (2011) Expert Rev Vaccines 10 (4): 499-511). CpG DNA as a vaccine adjuvant. When used as vaccine adjuvants, CpG ODNs improve the function of professional antigen-presenting cells and boost the generation of humoral and cellular vaccine-specific immune responses. In some embodiments, CpG can be administered in combination with the genetically engineered bacteria of the invention.

In one embodiment, the genetically engineered micororganisms are administered in combination with tumor cell lysates.

The dosage of the pharmaceutical composition and the frequency of administration may be selected based on the severity of the symptoms and the progression of the cancer. The appropriate therapeutically effective dose and/or frequency of administration can be selected by a treating clinician.

### Treatment In Vivo

The genetically engineered bacteria may be evaluated in vivo, e.g., in an animal model. Any suitable animal model of a disease or condition associated with cancer may be used, e.g., a tumor syngeneic or xenograft mouse models (see, e.g., Yu et al., 2015). The genetically engineered bacteria may be administered to the animal systemically or locally, e.g., via oral administration (gavage), intravenous, or subcutaneous injection or via intratumoral injection, and treatment efficacy determined, e.g., by measuring tumor volume.

Non-limiting examples of animal models include mouse models, as described in Dang et al., 2001, Heap et al., 2014 and Danino et al., 2015).

Pre-clinical mouse models determine which immunotherapies and combination immunotherapies will generate the optimal therapeutic index (maximal anti-tumor efficacy and minimal immune related adverse events (irAEs)) in different cancers.

Implantation of cultured cells derived from various human cancer cell types or a patient's tumor mass into mouse tissue sites has been widely used for generations of cancer mouse models (xenograft modeling). In xenograft modeling, human tumors or cell lines are implanted either subcutaneously or orthotopically into immune-compromised host animals (e.g., nude or SCID mice) to avoid graft rejection. Because the original human tumor microenvironment is not recapitulated in such models, the activity of anti-cancer agents that target immune modulators may not be accurately measured in these models, making mouse models with an intact immune system more desirable.

Accordingly, implantation of murine cancer cells in a syngeneic immunocompetent host (allograft) are used to generate mouse models with tumor tissues derived from the same genetic background as a given mouse strain. In syngeneic models, the host immune system is normal, which may more closely represent the real life situation of the tumor's micro-environment. The tumor cells or cancer cell lines are implanted either subcutaneously or orthotopically into the syngeneic immunocompetent host animal (e.g., mouse). Representative murine tumor cell lines, which can be used in syngeneic mouse models for immune checkpoint benchmarking include, but are not limited to the cell lines listed in Table 32.

TABLE 32
Selected cell lines for use in syngeneic mouse models

| Cancer Types | Cell LInes |
|---|---|
| Bladder | MBT-2 |
| Breast | 4T1, EMT6, JC |
| Colon | CT-26, Colon26, MC38 |
| Kidney | Renca |
| Leukemia | L1210, C1498 |
| Mastocytoma P815 | P815 |
| Neuroblastoma Neuro -2-A | Neuro-2a |
| Myeloma | MPC-11 |
| Liver | H22 |
| Lung | LL/2, KLN205 |
| Lymphoma | A20, EL4, P388D1, L15178-R, E.G7- |
| | OVA |
| Melanoma | B16-BL6, B16-F10, S91 |
| Pancreatic | Pan02 |
| Prostate | RM-1 |
| Fibrosarcoma | WHI-164 |
| Plasmacytoma | J558 |

Additional cell lines include, but are not limited to those in Table 33, which are described with respect to CTLA-4 benchmarking in Joseph F. Grosso and Maria N. Jure-Kunkel et al., 2013, the contents of which is herein incorporated by reference in its entirety.

TABLE 33
Murine cell lines and CTLA-4 antibodies
for syngenic mouse models

| Murine | Tumor type/Mouse | Anti-CTLA-4 |
|---|---|---|
| Tumor | strain | Ab/Tx regimen |
| Brain | SMA-560 | 9H10; d7* (100 μg), d10 |
| | Glioma/Vm/Dk) | (50 μg), d13 (50 μg) |
| | | post-implant |
| | GL-261 | 9H10; d0 (100 μg), d3 (50 |
| | Glioma/C57BL/6) | μg), d6 (50 μg), |
| Ovarian | OV-HM/C57BL/6 × | UC10-4F10-11; |
| | C3H/He) | 1 mg/mouse |
| Bladder | MB49/C57BL/6 | 9D9; d7, d10, d13 |
| | | (200 μg each) |
| Sarcoma | Meth-A/BALB/c | 9H10; d6 (100 μg), d9 |
| | | (50 μg), d12 (50 μg) |
| | MC38, 11A1 | 9H10; d14 (100 μg), d17 |
| | BALB/c, C57BL/6 | (50 μg), d20 (50 μg) |
| Breast | TSA/BALB/c (62 | 9H10; d12, d14, d16 |
| | | (200 μg each) |
| | 4T1 BALB/c | 9H10; d14, d18, d21 |
| | | (200 μg each) |
| | 4T1 BALB/c | 9H10; d14, d18, d21 |
| | | (200 μg each) |
| | 4T1 BALB/c | UC10-4F10-11; d7, d11, |
| | | d15, d19 (100 μg each) |
| | SM1/BALB/c | 9H10; d4, d7, d10 |
| | | (100 μg each) |
| | EMT6/BALB/c | UC10-4F10-11; d4, d8, d12 |
| | | (400 μg each) Ixa: d3, d7, |
| | | d11 |
| Colon | MC38/C57BL/6 | UC10-4F10-11; d7, d11, |
| | | d16 (100 μg each) |
| | MC38 | K4G4, L1B11, L3D10 |
| | CT26 BALB/c | 9H10; d10 (100 μg), d13 |
| | | (50 μg), d15 (50 μg) |
| | CT26 BALB/c | UC10-4F10-11; d5, d9, d13 |
| | | (400 μg each) Ixa: d4, d8, |
| | | d12 |
| | MC38/C57BL/6 | UC10-4F10-11; d14, d21, |
| | | d28 (800 μg each) |
| Lymphoma | BW5147.3/AKR | UC10-4F10-11; d-1 |
| | | (250 μg), d0 (250 μg), |
| | | d4 [100 μg), d8 (100 μg), |
| | | d12 (100 μg) |
| | EL4/C57BL/6 | 9H10; d3, d5 |
| | | (100 μg each) |
| Fibrosarcoma | SA1N/A/J | 9H10; every 4 days |
| | | (200 μg each) |
| | SA1N | UC10-4F10-11; d12, d16, |
| | | d20 (400 μg each) Ixa: |
| | | d11, d15, d15 |
| Prostata | TRAMP | 9H10; d7, d10, d13 |
| | C1[pTC1]/C57BU6 | (100 μg each) |
| | TRAMP | 9H10; d4, d7, d10 |
| | C2/C57BL/6 | (100 μg each) |
| | TRAMP/C57BL | 9H10; 14-16 week old mice |
| | | d7, d10, d16 post-tR tx |
| | | (100 μg each) |
| | TRAMP | 9H10; d29, d33, d40, d50 |
| | C2/C57BL/6 | (100 μg each) d29 = 1d |
| | | post-cryoablation |
| Melanoma | B16/C57BL/6 | 9H10; d0, d3, d6 |
| | | (200 μg each) |
| | B16/C57BL/6 | 9H10; d6 (100 μg), d8 |
| | | [50 μg), d10 (50 μg) |
| | B16/C57BL/6 | 9D9; d3, d6, d9 |
| | B16/C57BL/6 | 9H10; d3, d6, d9 |
| | | (100 μg each) |
| | B16.F10/ | 9H10; d5 (100 μg), d7 |
| | C57BL/6 | (50 μg), d9 (50 μg) |
| Lung | M109/BALB/c | UC10-4F10-11; d4, d8, |
| | | d12(400 μg each) |
| | | Ixa: d3, d7, d11 |
| Plasmacytoma | MOPC-315/ | UC10-4F10-11; 20 mm |
| | BALB/cANnCrlBr | tumors tx daily for 10 days |
| | | (100 μg each) |

For tumors derived from certain cell lines, ovalbumin can be added to further stimulate the immune response, thereby increasing the response baseline level.

Examples of mouse strains that can be used in syngeneic mouse models, depending on the cell line include C57BL/6, FVB/N, Balb/c, C3H, HeJ, C3H/HeJ, NOD/ShiLT, A/J, 129S1/SvlmJ, NOD. Additionally, several further genetically engineered mouse strains have been reported to mimic human tumorigenesis at both molecular and histologic levels. These genetically engineered mouse models also provide excellent tools to the field and additionally, the cancer cell lines derived from the invasive tumors developed in these models are also good resources for cell lines for syngeneic tumor models Examples of genetically engineered strains are provided in Table 34.

TABLE 34
Exemplary genetic engineered mouse strains of interest

| | | Predicted |
|---|---|---|
| | Strain | cancer |
| Animal strain | background | type |
| C57BL/6- | C57BL/6 | Prostate cancer |
| Tg(TRAMP)8247Ng/JNju | | |
| FVB/N-Tg□MMTV- | FVB/N | Breast cancer |
| PyVT)634Mul/Jnju | | |
| C57BL/6J-Apc^{Min}/JNju | C57BL/6 | Colorectal cancer |
| STOCK | C57BL/6JNju | Medulloblastoma |
| Ptch^{1tm1Mps}/JNju | | |
| NOD- | NOD/ShiLt | Not specific |
| Prkdc^{em26Cd52}Il2rg^{em26Cd22}Nju | | |
| C57BL/6J-Apc^{Min}/JNju | C57BL/6 | Colorectal cancer |
| BALB/cJNju | BALB/c | Lung cancer |
| C3H/HeJNju (Urethane | C3H/HeJ | Lung cancer |
| induced lung cancer model) | | |
| A/JNju | A/J | Lung cancer |
| A/Jnju (Urethane induced | A/J | Lung cancer |
| lung cancer model) | | |
| C3H/HeJSlac | C3H/HeJ | Lung cancer |
| 129S1/SvImJNju (Urethane | 129S1/SvImJ | Lung cancer |
| induced lung cancer model) | | |
| Kras^{LSL-G12D/WT} | C57BL/6 | Lung cancer |
| Kras^{LSL-G12D/WT}; | C57BL/6 | Lung cancer |
| P53-^{KO/KO} | | |
| Pdx1-cre; Kras^{LSL-G12D/WT}; | C57BL/6 | Pancreatic cancer |
| P53^{KO/} KO | | |
| Kras^{LSL-G12D/WT}; | C57BL/6; | Pancreaticc cancer; |
| P16^{KO/KO} | FVB/N | Lung cancer |
| Kras^{LSL-G12D/WT}; | C57BL/6 | Ovarian cancer; |
| PTEN^{CKO/CKO} | | Prostate cancer; |
| | | Brain cancer |
| Pbsn-cre; Kras^{LSL-G12D/WT}; | C57BL/6 | Prostate cancer |
| PTEN^{CKO/CKO} | | |
| P53^{KO/KO}; | C57BL/6 | Prostate cancer |
| PTEN^{CKO/CKO} | | |
| Pbsn-cre; PTEN^{CKO/CKO} | C57BL/6 | Prostate cancer |
| NOD | NOD | Leukemia |
| B6.Cg- | C57BL/6 | B cell Lymphoma |
| Tg(IghMyc)22Bri/JNju | | |
| PTEN^{CKO/CKO} | C57BL/6 | Ovarian cancer |
| | | (Female); Prostate |
| | | cancer (Male); Tes/s |
| | | cancer (Male) |
| NASH-HCC (Streptozotocin | C57BL/6 | Hepatocellular |
| and high-fat diet induced liver | | Carcinoma |
| cancer model) | | |
| BALB/c nude | BALB/c | Not specific |
| C3H/He | C3H/He | Hepatocellular |
| | | Carcinoma |
| B6N | C57BL/6 | Not specific |
| B6/N-Akr1c12^{tm1a}Nju | C57BL/6 | Not specific |
| P53 null from VitalStar | C57BL/6 | Not specific |
| P53 null from VitalStar | C57BL/6 | Not specific |
| P53 null from VitalStar | C57BL/6 | Not specific |
| Pdx1-cre; Kras^{LSL-G12D/WT}; | C57BL/6 | Pancrea/c cancer |
| P53^{KO/} KO | | |
| Kras^{LSL-G12D/WT}; | C57BL/6; | Pancrea/c cancer; |
| P16^{KO/KO} | FVB/N | Lung cancer |
| Kras^{LSL-G12D/WT}; | C57BL/6 | Ovarian cancer; |
| PTEN^{CKO/CKO} | | |
| Kras^{LSL-G12D/WT}; | C57BL/6 | Prostate cancer; |
| PTEN^{CKO/CKO} | | |
| Kras^{LSL-G12D/WT}; | C57BL/6 | Brain cancer |
| PTEN^{CKO/CKO} | | |
| Pbsn-cre; Kras^{LSL-G12D/WT}; | C57BL/6 | Prostate cancer |
| PTEN^{CKO/CKO} | | |
| P53^{KO/KO}; PTEN^{CKO/CKO} | C57BL/6 | Prostate cancer |
| Pbsn-cre;PTEN^{CKO/CKO} | C57BL/6 | Prostate cancer |
| Kras^{LSL-G12D/WT} | C57BL/6 | Lung cancer |
| NOD | NOD | Leukemia |
| B6.Cg- | C57BL/6 | B cell Lymphoma |
| Tg(IghMyc)22Bri/JNju | | |
| PTEN^{CKO/CKO} | C57BL/6 | Ovarian cancer |
| | | (Female); Prostate |
| | | cancer (Male); Tes/s |
| | | cancer (Male) |
| NASH-HCC (Streptozotocin | C57BL/6 | Hepatocellular |
| and high-fat diet induced | | Carcinoma |
| liver cancer model) | | |
| BALB/c nude | BALB/c | Not specific |
| C3H/He | C3H/He | Hepatocellular |
| | | Carcinoma |
| B6N | C57BL/6 | Not specific |
| B6/N-Akr1c12^{tm1a}Nju | C57BL/6 | Not specific |
| P53 null from VitalStar | C57BL/6 | Not specific |
| P53 null from VitalStar | C57BL/6 | Not specific |
| P53 null from VitalStar | C57BL/6 | Not specific |
| Kras^{LSL-G12D/WT}; | C57BL/6 | Not specific |
| P53^{KO/KO} | | |

Often antibodies directed against human proteins do not detect their murine counterparts. In studying antibodies, including those directed against human immune checkpoint molecules, it is necessary to take this in consideration. For example, Ipilimumab did not show cross-reactivity with or binding to CTLA-4 from rats, mice or rabbits.

In some cases, mice transgenic for the gene of interest can used to overcome this issue, as was done for ipilimumab. However, in syngeneic mouse models without a human transgene, mouse protein reactive antibodies must be used to test therapeutic antibody strategies. For example, suitable CTLA-4 antibodies for expression by the genetically engineered bacteria of interest include, but are not limited to, 9H10, UC10-4F10-11, 9D9, and K4G4 (Table 33).

More recently, “humanized” mouse models have been developed, in which immunodeficient mice are reconstituted with a human immune system, and which have helped overcome issues relating to the differences between the mouse and human immune systems, allowing the in vivo study of human immunity. Severely immunodeficient mice which combine the IL2receptor null and the severe combined immune deficiency mutation (scid) (NOD-scid IL2Rgnull mice) lack mature T cells, B cells, or functional NK cells, and are deficient in cytokine signaling. These mice can be engrafted with human hematopoietic stem cells and peripheral-blood mononuclear cells. CD34+ hematopoietic stem cells (hu-CD34) are injected into the immune deficient mice, resulting in multi-lineage engraftment of human immune cell populations including very good T cell maturation and function for long-term studies. This model has a research span of 12 months with a functional human immune system displaying T-cell dependent inflammatory responses with no donor cell immune reactivity towards the host. Patient derived xenografts can readily be implanted in these models and the effects of immune modulatory agents studied in an in vivo setting more reflective of the human tumor microenvironment (both immune and non-immune cell-based) (Baia et al., 2015).

Human cell lines of interest for use in the humanized mouse models include but are not limited to HCT-116 and HT-29 colon cancer cell lines.

A rat F98 glioma model and the utility of spontaneous canine tumors, as described in Roberts et al 2014, the contents of each of which are herein incorporated by reference in their entireties. Locally invasive tumors generated by implantation of F98 rat glioma cells engineered to express luciferase were intratumorally injected with C. novyi-NT spores, resulting in germination and a rapid fall in luciferase activity. C. novyi-NT germination was demonstrated by the appearance of vegetative forms of the bacterium. In these studies, C. novyi-NT precisely honed to the tumor sparing neighboring cells.

Canine soft tissue sarcomas for example are common in many breeds and have clinical, histopathological, and genetically features similar to those in humans (Roberts et al, 2014; Staedtke et al., 2015), in particular, in terms of genetic alterations and spectrum of mutations. Roberts et al. conducted a study in dogs, in which C. novyi-NT spores were intrtatumorally injected (1×10⁸ C. novyi-NT spores) into spontaneously occurring solid tumors in one to 4 treatment cycles and followed for 90 days. A potent inflammatory response was observed, indicating that the intrattumoral injections mounted an innate immune response.

In some embodiments, the genetically engineered microorganisms of the invention are administered systemically, e.g., orally, subcutaneously, intravenously or intratumorally into any of the models described herein to assess anti-tumor efficacy and any treatment related adverse side effects.

### EXAMPLES

### Example 1. Phenylalanine Transporter-Integration of PheP into the Bacterial Chromosome

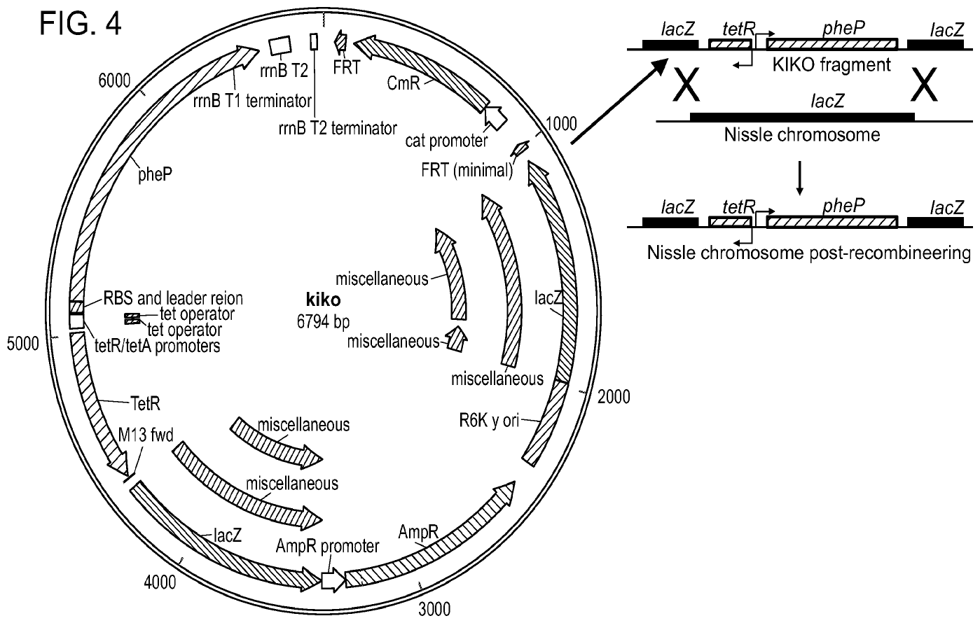

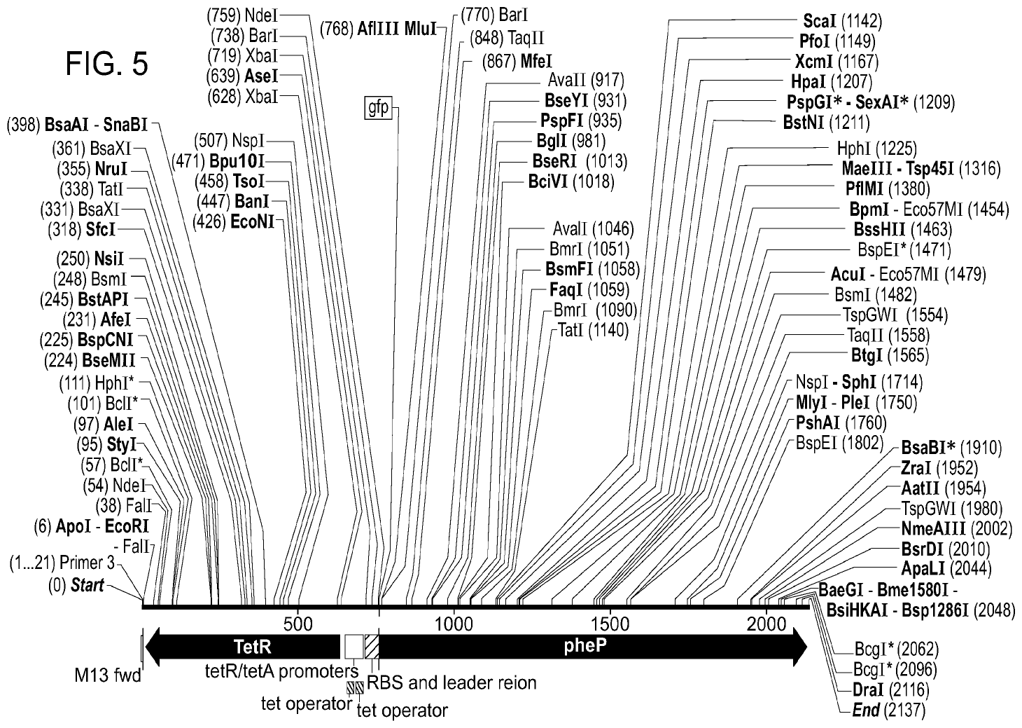

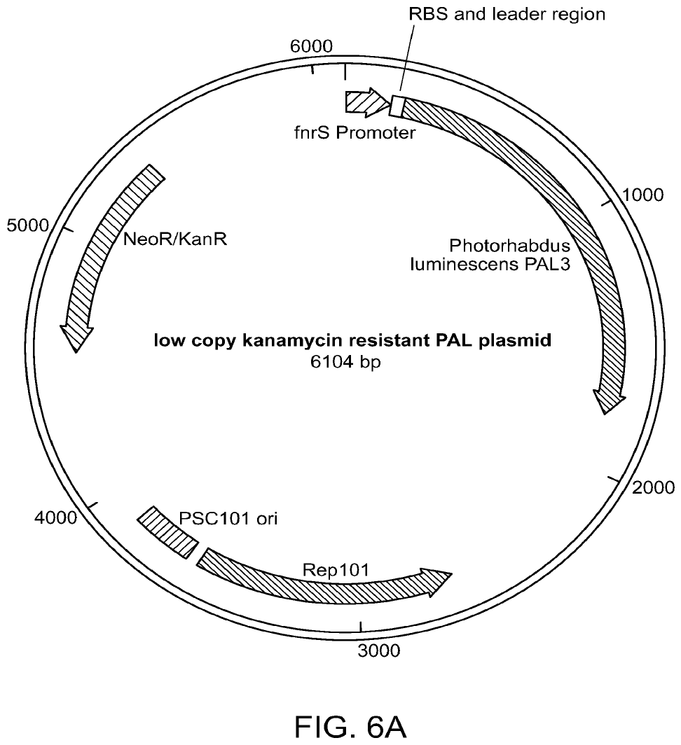

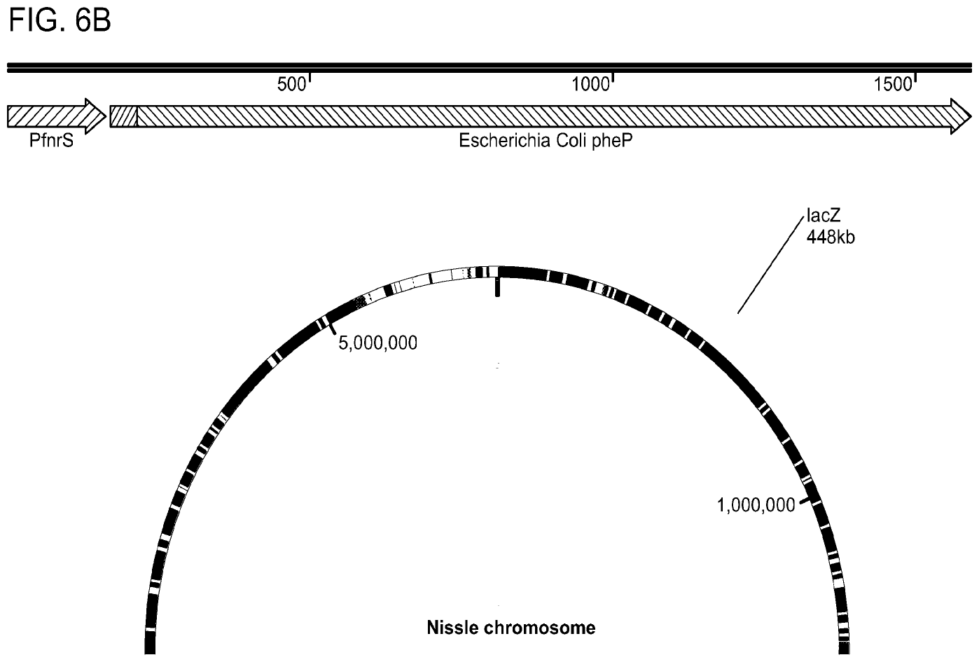

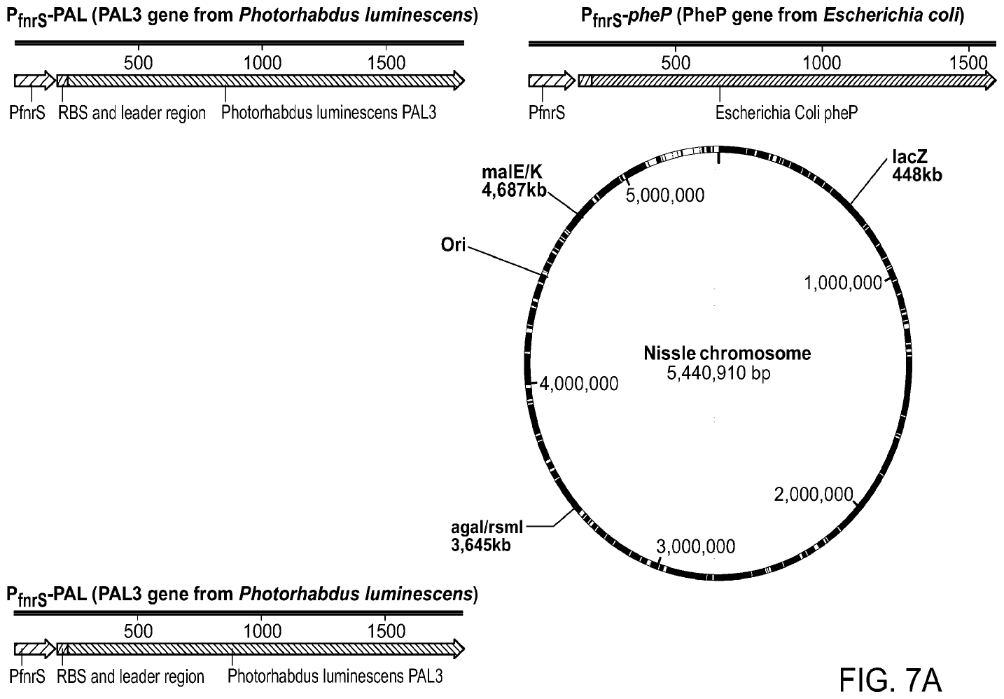

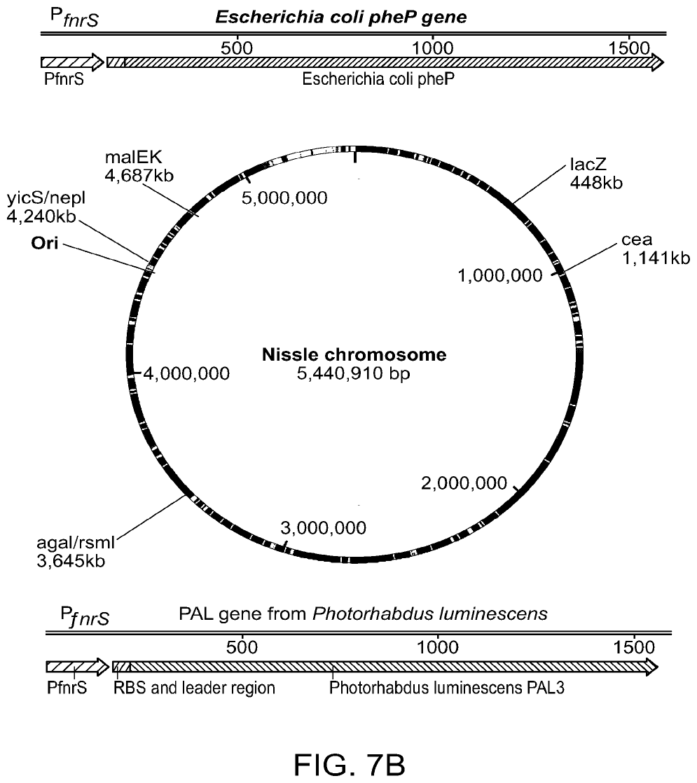

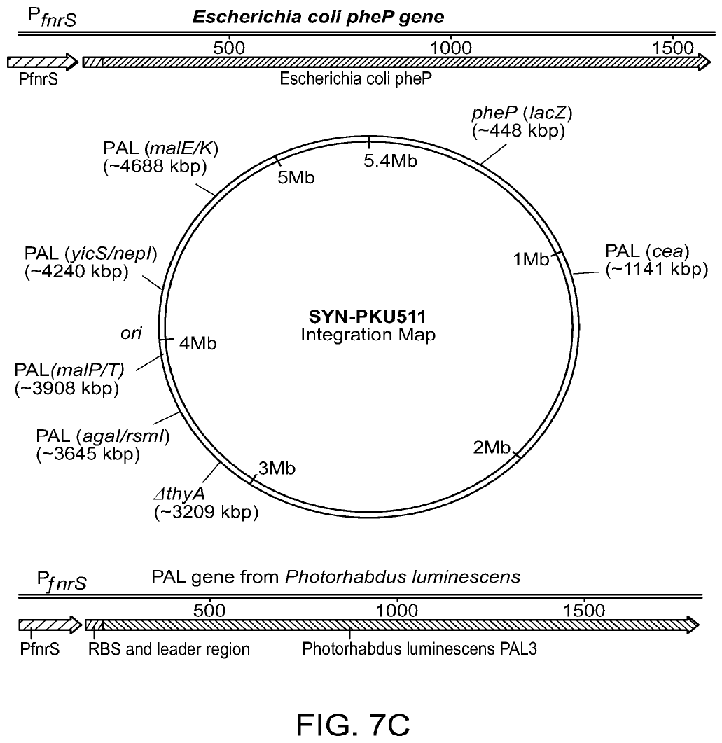

In some embodiments, it may be advantageous to increase phenylalanine transport into the cell, thereby enhancing phenylalanine metabolism. Therefore, a second copy of the native high affinity phenylalanine transporter, PheP, driven by an inducible promoter, was inserted into the Nissle genome through homologous recombination. Organization of the construct is shown in FIG. 4. The pheP gene was placed downstream of the Pₜₑₜ promoter, and the tetracycline repressor, TetR, was divergently transcribed (see, e.g., FIG. 4). This sequence was synthesized by Genewiz (Cambridge, MA). To create a vector capable of integrating the synthesized TetR-PheP construct into the chromosome, Gibson assembly was first used to add 1000 bp sequences of DNA homologous to the Nissle lacZ locus into the R6K origin plasmid pKD3. This targets DNA cloned between these homology arms to be integrated into the lacZ locus in the Nissle genome. Gibson assembly was used to clone the TetR-PheP fragment between these arms. PCR was used to amplify the region from this plasmid containing the entire sequence of the homology arms, as well as the pheP sequence between them. This PCR fragment was used to transform electrocompetent Nissle-pKD46, a strain that contains a temperature-sensitive plasmid encoding the lambda red recombinase genes. After transformation, cells were grown for 2 hrs before plating on chloramphenicol at 20 μg/mL at 37° C. Growth at 37° C. cures the pKD46 plasmid. Transformants containing anhydrous tetracycline (ATC)-inducible pheP were lac-minus (lac-) and chloramphenicol resistant.

### Example 2. Effect of the Phenylalanine Transporter on Phenylalanine Degradation

To determine the effect of the phenylalanine transporter on phenylalanine degradation, phenylalanine degradation and trans-cinnamate accumulation achieved by genetically engineered bacteria expressing PAL1 or PAL3 on low-copy (LC) or high-copy (HC) plasmids in the presence or absence of a copy of pheP driven by the Tet promoter integrated into the chromosome was assessed.

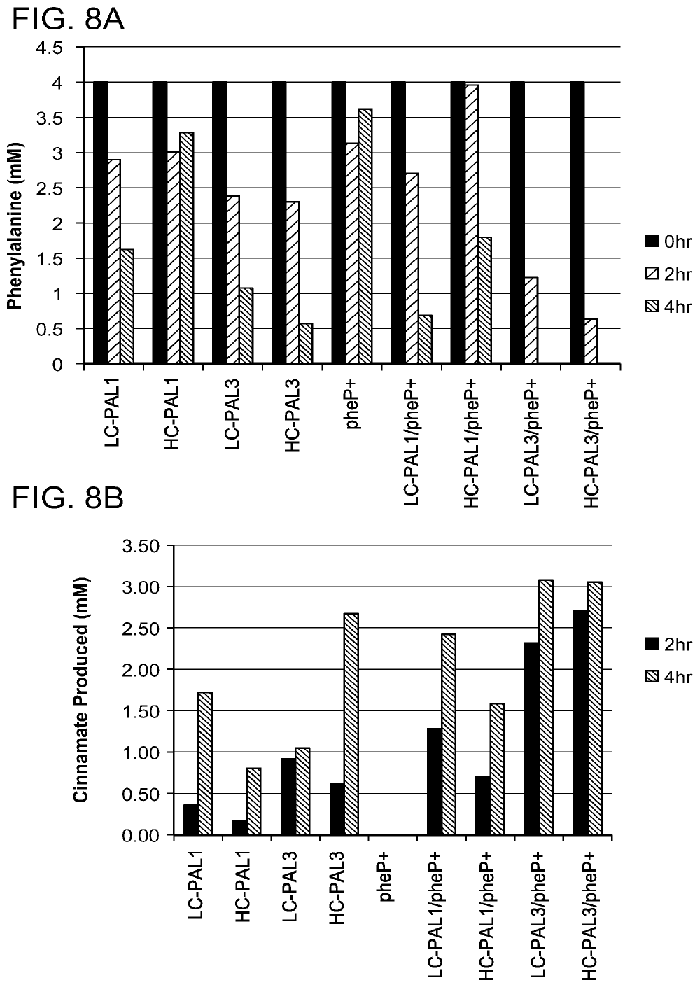

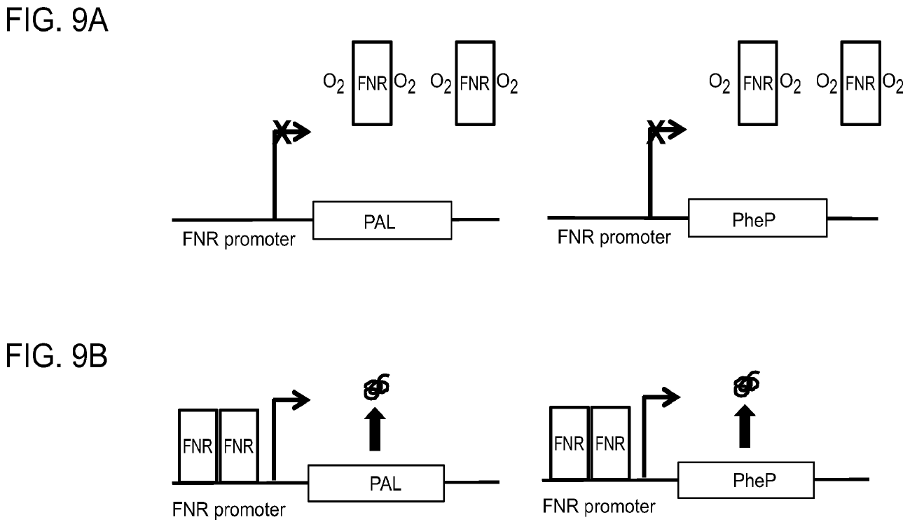

For in vitro studies, all incubations were performed at 37° C. Cultures of E. coli Nissle transformed with a plasmid comprising the PAL gene driven by the Tet promoter were grown overnight and then diluted 1:100 in LB. The cells were grown with shaking (200 rpm) to early log phase. Anhydrous tetracycline (ATC) was added to cultures at a concentration of 100 ng/mL to induce expression of PAL, and bacteria were grown for another 2 hrs. Bacteria were then pelleted, washed, and resuspended in minimal media, and supplemented with 4 mM phenylalanine. Aliquots were removed at 0 hrs, 2 hrs, and 4 hrs for phenylalanine quantification (FIG. 8A), and at 2 hrs and 4 hrs for cinnamate quantification (FIG. 8B), by mass spectrometry, as described in Examples 24-26. As shown in FIG. 8, expression of pheP in conjunction with PAL significantly enhances the degradation of phenylalanine as compared to PAL alone or pheP alone. Notably, the additional copy of pheP permitted the complete degradation of phenylalanine (4 mM) in 4 hrs (FIG. 8A). FIG. 8B depicts cinnamate levels in samples at 2 hrs and 4 hrs post-induction. Since cinnamate production is directly correlated with phenylalanine degradation, these data suggest that phenylalanine disappearance is due to phenylalanine catabolism, and that cinnamate may be used as an alternative biomarker for strain activity. PheP overexpression improves phenylalanine metabolism in engineered bacteria.

In conclusion, in conjunction with pheP, even low-copy PAL-expressing plasmids are capable of almost completely eliminating phenylalanine from a test sample (FIGS. 8A and 8B). Furthermore, without wishing to be bound by theory, in some embodiments, that incorporate pheP, there may be additional advantages to using a low-copy PAL-expressing plasmid in conjunction in order to enhance the stability of PAL expression while maintaining high phenylalanine metabolism, and to reduce negative selection pressure on the transformed bacterium. In alternate embodiments, the phenylalanine transporter is used in conjunction with a high-copy PAL-expressing plasmid.

### Example 3. Phenylalanine Degradation in Recombinant E. coli with and without pheP Overexpression

The SYN-PKU304 and SYN-PKU305 strains contain low-copy plasmids harboring the PAL3 gene, and a copy of pheP integrated at the lacZ locus. The SYN-PKU308 and SYN-PKU307 strains also contain low-copy plasmids harboring the PAL3 gene, but lack a copy of pheP integrated at the lacZ locus. In all four strains, expression of PAL3 and pheP (when applicable) is controlled by an oxygen level-dependent promoter.

To determine rates of phenylalanine degradation in engineered E. coli Nissle with and without pheP on the chromosome, overnight cultures of SYN-PKU304 and SYN-PKU307 were diluted 1:100 in LB containing ampicillin, and overnight cultures of SYN-PKU308 and SYN-PKU305 were diluted 1:100 in LB containing kanamycin. All strains were grown for 1.5 hrs before cultures were placed in a Coy anaerobic chamber supplying 90% N₂, 5% CO₂, and 5% H₂. After 4 hrs of induction, bacteria were pelleted, washed in PBS, and resuspended in 1 mL of assay buffer. Assay buffer contained M9 minimal media supplemented with 0.5% glucose, 8.4% sodium bicarbonate, and 4 mM of phenylalanine.

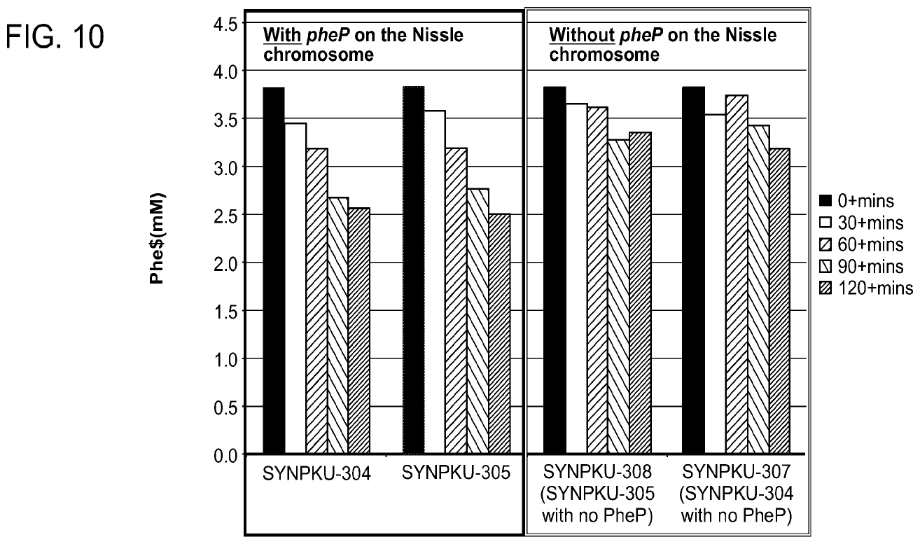

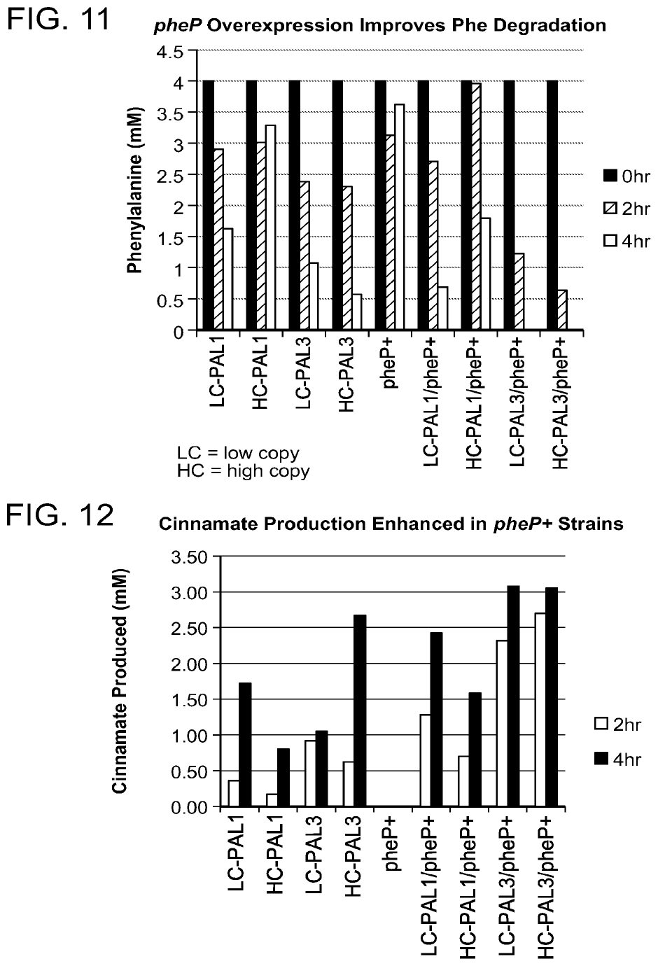

For the activity assay, starting counts of colony-forming units (cfu) were quantified using serial dilution and plating. Aliquots were removed from each cell assay every 30 min for 3 hrs for phenylalanine quantification by mass spectrometry. Specifically, 150 μL of bacterial cells were pelleted and the supernatant was harvested for LC-MS analysis, with assay media without cells used as the zero-time point. FIG. 10 shows the observed phenylalanine degradation for strains with pheP on the chromosome (SYN-PKU304 and SYN-PKU305; left), as well as strains lacking pheP on the chromosome (SYN-PKU308 and SYN-PKU307; right). These data show that pheP overexpression is important in order to increase rates of phenylalanine degradation in synthetic probiotics.

### Strains Used in the Experiments

| | | | PAL |
|---|---|---|---|
| | | | Activity |
| Strain | Strain | | (umol/hr/ |
| Name | Name | Genotype | 10\hat{ }9 cells) |
| SYN025 | SYN-PKU101 | Low copy pSC101- | ND |
| | | Ptet::PAL1, ampicillin | |
| | | resistant | |
| SYN026 | SYN-PKU102 | High copy pColE1- | ND |
| | | Ptet::PAL1, ampicillin | |
| | | resistant, | |
| SYN065 | SYN-PKU201 | Low copy pSC101- | ND |
| | | Ptet::PAL3, ampicillin | |
| | | resistant | |
| SYN063 | SYN-PKU202 | High copy pColE1- | ND |
| | | Ptet::PAL3, ampicillin | |
| | | resistant, | |
| SYN107 | SYN-PKU203 | lacZ::Ptet-pheP::cam | 0 |
| SYN108 | SYN-PKU401 | Low copy pSC101- | 1.1 |
| | | Ptet::PAL1, ampicillin | |
| | | resistant, chromosomal | |
| | | lacZ::Ptet-pheP::cam | |
| SYN109 | SYN-PKU402 | High copy pColE1- | 0.8 |
| | | Ptet::PAL1, ampicillin | |
| | | resistant, chromosomal | |
| | | lacZ::Ptet-pheP::cam | |
| SYN110 | SYN-PKU302 | Low Copy pSC101- | 2.2 |
| | | Ptet::PAL3, ampicillin | |
| | | resistant; chromosomal | |
| | | lacZ::Ptet-pheP::cam | |
| SYN111 | SYN-PKU303 | High copy pColE1- | 7.1 |
| | | Ptet::PAL3, ampicillin | |
| | | resistant, chromosomal | |
| | | lacZ::Ptet-pheP::cam | |
| SYN340 | SYN-PKU304 | Low Copy pSC101- | 3 |
| | | PfnrS::PAL3, ampicillin | |
| | | resistant; chromosomal | |
| | | lacZ::PfnrS-pheP::cam | |
| SYN958 | SYN-PKU305 | Low Copy pSC101- | 3 |
| | | PfnrS::PAL3, | |
| | | kanamycin resistant; | |
| | | chromosomal | |
| | | lacZ::PfnrS-pheP::cam | |
| SYN959 | SYN-PKU307 | Low Copy pSC101- | 0.3 |
| | | PfnrS::PAL3, ampicillin | |
| | | resistant; | |
| SYN837 | SYN-PKU308 | Low Copy pSC101- | 0.3 |
| | | PfnrS::PAL3, | |
| | | kanamycin resistant; | |

### Example 4. Construction of Plasmids Encoding Branched Chain Amino Acid Importers and Branched Chain Amino Acid Catabolism Enzyme

The kivD gene of Lactococcus lactis IFPL730 was synthesized (Genewiz), fused to the Tet promoter, cloned into the high-copy plasmid pUC57-Kan by Gibson assembly and transformed into E. coli DH5a to generate the plasmid pTet-kivD. The bkd operon of Pseudomonas aeruginosa PAO1 fused to the Tet promoter was synthesized (Genewiz) and cloned into the high-copy plasmid pUC57-Kan to generate the plasmid pTet-bkd. The bkd operon of Pseudomonas aeruginosa PAO1 fused to the Idh gene from PA01 and the Tet promoter was synthesized (Genewiz) and cloned into the high-copy plasmid pUC57-Kan to generate the plasmid pTet-Idh-bkd. The livKHMGF operon from E. coli Nissle fused to the Tet promoter was synthesized (Genewiz), cloned into the pKIKO-lacZ plasmid by Gibson assembly and transformed into E. coli PIR1 as described in Example 3 to generate the pTet-livKHMGF.

### Example 5. Generation of Recombinant Bacterial Cell Comprising a Genetic Modification that Reduces Export of a Branched Chain Amino Acid

E. coli Nissle was transformed with the pKD46 plasmid encoding the lambda red proteins under the control of an arabinose-inducible promoter as follows. An overnight culture of E. coli Nissle grown at 37° C. was diluted 1:100 in 4 mL of lysogeny broth (LB) and grown at 37° C. until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 μL of a pKD46 miniprep was added to the cells, mixed by pipetting, and pipetted into a sterile, chilled 1 mm cuvette. The cuvette was placed into the sample chamber, and the electric pulse was applied. 500 μL of room-temperature SOC media was immediately added, and the mixture was transferred to a culture tube and incubated at 30° C. for 1 hr. The cells were spread out on an LB plate containing 100 μg/mL carbenicillin and incubated at 30° C.

A ΔleuE deletion construct with 77 bp and a 100 bp flanking leuE homology regions and a kanamycin resistant cassette flanked by FRT recombination site was generated by PCR, column-purified and transformed into E. coli Nissle pKD46 as follows. An overnight culture of E. coli Nissle pKD46 grown in 100 μg/mL carbenicillin at 30° C. was diluted 1:100 in 5 mL of LB supplemented with 100 μg/mL carbenicillin, 0.15% arabinose and grown until it reaches an OD₆₀₀ of 0.4-0.6. The bacteria were aliquoted equally in five 1.5 mL microcentrifuge tubes, centrifuged at 13,000 rpm for 1 min and the supernatant was removed. The cells were then washed three times in pre-chilled 10% glycerol and combined in 50 uL pre-chilled 10% glycerol. The electroporator was set to 1.8 kV. 2 μL of a the purified ΔleuE deletion PCR fragment are then added to the cells, mixed by pipetting, and pipetted into a sterile, chilled 1 mm cuvette. The cuvette was placed into the sample chamber, and the electric pulse was applied. 500 μL of room-temperature SOC media was immediately added, and the mixture was transferred to a culture tube and incubated at 37° C. for 1 hr. The cells were spread out on an LB plate containing 50 μg/mL kanamycin. Five kanamycin-resistant transformants were then checked by colony PCR for the deletion of the leuE locus.

The kanamycin cassette was then excised from the ΔleuE deletion strain as follows. ΔleuE was transformed with the pCP20 plasmid encoding the Flp recombinase gene. An overnight culture of ΔleuE grown at 37° C. in LB with 50 μg/mL kanamycin was diluted 1:100 in 4 mL of LB and grown at 37° C. until it reaches an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 μL of a pCP20 miniprep was added to the cells, mixed by pipetting, and pipetted into a sterile, chilled 1 mm cuvette. The dry cuvette was placed into the sample chamber, and the electric pulse was applied. 500 μL of room-temperature SOC media was immediately added, and the mixture was transferred to a culture tube and incubated at 30° C. for 1 hr. The cells were spread out on an LB plate containing 100 μg/mL carbenicillin and incubated at 30° C. Eight transformants were then streaked on an LB plate and were incubated overnight at 43° C. One colony per transformant was picked and resuspended in 10 uL LB and 3 μL of the suspension were pipetted on LB, LB with 50 μg/mL Kanamycin or LB with 100 μg/mL carbenicillin. The LB and LB Kanamycin plates were incubated at 37° C. and the LB Carbenicillin plate was incubated at 30° C. Colonies showing growth on LB alone were selected and checked by PCR for the excision of the Kanamycin cassette.

### Example 6. Generation of Recombinant Bacteria Comprising an Importer of a Branched Chain Amino Acid and/or a Branched Chain Amino Acid Catabolism Enzyme and Lacking an Exporter of a Branched Chain Amino Acid

pTet-kivD, pTet-bkd, pTet-ldh-bkd and pTet-livKHFGF plasmids described above were transformed into E. coli Nissle (pTet-kivD), Nissle (pTet-kivD, pTet-bkd, pTet-ldh-bkd), DH5a (pTet-kivD, pTet-bkd, pTet-ldh-bkd) or PIR1 (pTet-livKHMGF). All tubes, solutions, and cuvettes were pre-chilled to 4° C. An overnight culture of E. coli (Nissle, ΔleuE, DH5α or PIR1) was diluted 1:100 in 4 mL of LB and grown until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pTet-kivD, pTet-bkd, pTet-ldh-bkd or pTet-livKHMGF miniprep was added to the cells, mixed by pipetting, and pipetted into a sterile, chilled 1 mm cuvette. The dry cuvette was placed into the sample chamber, and the electric pulse was applied. 500 μL of room-temperature SOC media was immediately added, and the mixture was transferred to a culture tube and incubated at 37° C. for 1 hr. The cells were spread out on an LB plate containing 50 ug/mL Kanamycin for pTet-kivD, pTet-bkd and pTet-ldh-bkd or 100 ug/mL carbenicillin for pTet-livKHMGF.

### Example 7. Generation of Recombinant Bacteria Comprising an Importer of a Branched Chain Amino Acid and a Genetic Modification that Reduces Export of a Branched Chain Amino Acid

E. coli Nissle ΔleuE was transformed with the pKD46 plasmid encoding the lambda red proteins under the control of an arabinose-inducible promoter as follows. An overnight culture of E. coli Nissle ΔleuE grown at 37° C. was diluted 1:100 in 4 mL of LB and grown at 37° C. until it reached an OD₆₀₀ of 0.4-0.6. 1 mL of the culture was then centrifuged at 13,000 rpm for 1 min in a 1.5 mL microcentrifuge tube and the supernatant was removed. The cells were then washed three times in pre-chilled 10% glycerol and resuspended in 40 uL pre-chilled 10% glycerol. The electroporator was set to 1.8 kV. 1 uL of a pKD46 miniprep was added to the cells, mixed by pipetting, and pipetted into a sterile, chilled 1 mm cuvette. The dry cuvette was placed into the sample chamber, and the electric pulse was applied. 500 μL of room-temperature SOC media was immediately added, and the mixture was transferred to a culture tube and incubated at 30° C. for 1 hr. The cells were spread out on an LB plate containing 100 μg/mL carbenicillin and incubated at 30° C.

The DNA fragment used to integrate Tet-livKHMGF into E. coli Nissle lacZ was amplified by PCR from the pTet-livKHMGF plasmid, column-purified and transformed into ΔleuE pKD46 as follows. An overnight culture of the E. coli Nissle ΔleuE pKD46 strain grown in LB at 30° C. with 100 μg/mL carbenicillin was diluted 1:100 in 5 mL of lysogeny broth (LB) supplemented with 100 μg/mL carbenicillin, 0.15% arabinose and grown at 30° C. until it reached an OD₆₀₀ of 0.4-0.6. The bacteria were aliquoted equally in five 1.5 mL microcentrifuge tubes, centrifuged at 13,000 rpm for 1 min and the supernatant was removed. The cells were then washed three times in pre-chilled 10% glycerol and combined in 50 uL pre-chilled 10% glycerol. The electroporator was set to 1.8 kV. 2 μL of a the purified Tet-livKHMGF PCR fragment were then added to the cells, mixed by pipetting, and pipetted into a sterile, chilled 1 mm cuvette. The dry cuvette was placed into the sample chamber, and the electric pulse was applied. 500 μL of room-temperature SOC media was immediately added, and the mixture was transferred to a culture tube and incubated at 37° C. for 1 hr. The cells were spread out on an LB plate containing 20 μg/mL chloramphenicol, 40 μg/mL X-Gal and incubated overnight at 37° C. White chloramphenicol resistant transformants were then checked by colony PCR for integration of Tet-livKHMGF into the lacZ locus.

### Example 8. Functional Assay Demonstrating that the Recombinant Bacterial Cells Decrease Branched Chain Amino Acid Concentration

For in vitro studies, all incubations were performed at 37° C. Cultures of E. coli Nissle ΔleuE, ΔleuE+pTet-kivD, ΔleuE+pTet-bkd, ΔleuE+pTet-Idh-bkd, ΔleuE lacZ:Tet-livKHMGF, ΔleuE lacZ:Tet-livKHMGF+pTet-kivD, ΔleuE lacZ:Tet-livKHMGF+pTet-bkd, ΔleuE lacZ:Tet-livKHMGF+pTet-ldh-bkd were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/ml to induce expression of KivD, Bkd, Ldh and LivKHFMG, and bacteria were grown for another 3 hours. Bacteria were then pelleted, washed, and resuspended in minimal media, and supplemented with 0.5% glucose and 2 mM leucine. Aliquots were removed at Oh, 1.5 h, 6 h and 18 h for leucine quantification by liquid chromatography-mass spectrometry (LCMS) using a Thermo TSQ Quantum Max triple quadrupole instrument. Briefly, 100 μL aliquots were centrifuged at 4,500 rpm for 10 min. 10 μL of the supernatant was resuspended in 90 uL water with 1 ug/mL L-leucine-5,5,5-d₃ (isotope used as internal standard). 10 μL of the samples was then resuspended in 90 uL 10% acetonitrile, 0.1% formic acid and placed in the LCMS autosampler. A C18 column 100×2 mm, 3 μm particles was used (Luna, Phenomenex). The mobile phases used were water 0.1% formic acid (solvent A) and acetonitrile 0.1% (solvent B). The gradient used was:
0 min: 95% A, 5% B
0.5 min: 95% A, 5% B
1 min: 10% A, 90% B
2.5 min: 10% A, 90% B
2.51 min: 95% A, 5% B
3.5 min: 95% A, 5% B

The Q1/Q3 transitions used for leucine and L-leucine-5,5,5-d₃ were 132.1/86.2 and 135.1/89.3 respectively.

Leucine was rapidly graded by the expression of kivD in the Nissle ΔleuE strain. After 6 h of incubation, leucine concentration dropped by over 99% in the presence of ATC. This effect was even more pronounced in the case of ΔleuE expressing both kivD and the leucine transporter livKHMGF where leucine is undetectable after 6 h of incubation. The expression of the bkd complex also leads rapidly to the degradation of leucine. After 6 h of incubation, 99% of leucine was degraded. The expression of the leucine transporter livKHMGF, in parallel with the expression of ldh and bkd leads to the complete degradation of leucine after 18 h.

### Example 9. Simultaneous Degradation of Branched Chain Amino Acids by Recombinant Bacteria Expressing a Branched Chain Amino Acid Catabolism Enzyme and an Importer of a Branched Chain Amino Acid

In these studies, all incubations were performed at 37° C. Cultures of E. coli Nissle, Nissle+pTet-kivD, ΔleuE+pTet-kivD, ΔleuE lacZ:Tet-livKHMGF+pTet-kivD were grown overnight in LB, LB 50 ug/mL Kanamycin or LB 50 ug/mL Kanamycin 20 ug/mL chloramphenicol and then diluted 1:100 in LB. The cells were grown with shaking (250 rpm) to early log phase with the appropriate antibiotics. Anhydrous tetracycline (ATC) was added to cultures at a concentration of 100 ng/ml to induce expression of KivD and LivKHFMG, and bacteria were grown for another 3 hours. Bacteria were then pelleted, washed, and resuspended in minimal media, and supplemented with 0.5% glucose and the three branched chain amino acids (leucine, isoleucine and valine, 2 mM each). Aliquots were removed at Oh, 1.5 h, 6 h and 18 h for leucine, isoleucine and valine quantification by liquid chromatography-mass spectrometry (LCMS) using a Thermo TSQ Quantum Max triple quadrupole instrument. Briefly, 100 μL aliquots were centrifuged at 4,500 rpm for 10 min. 10 μL of the supernatant was resuspended in 90 uL water with 1 ug/mL L-leucine-5,5,5-d₃ (isotope used as internal standard). 10 μL of the samples was then resuspended in water, 0.1% formic acid and placed in the LCMS autosampler. A C18 column 100×2 mm, 3 μm particles was used (Luna, Phenomenex). The mobile phases used were water 0.1% formic acid (solvent A) and acetonitrile 0.1% (solvent B). The gradient used was:
0 min: 100% A, 0% B
0.5 min: 100% A, 0% B
1.5 min: 10% A, 90% B
3.5 min: 10% A, 90% B
3.51 min: 100% A, 0% B
4.5 min: 100% A, 0% B

The Q1/Q3 transitions used are:
Leucine: 132.1/86.2
L-leucine-5,5,5-d₃: 135.1/89.3
Isoleucine: 132.1/86.2
Valine: 118.1/72

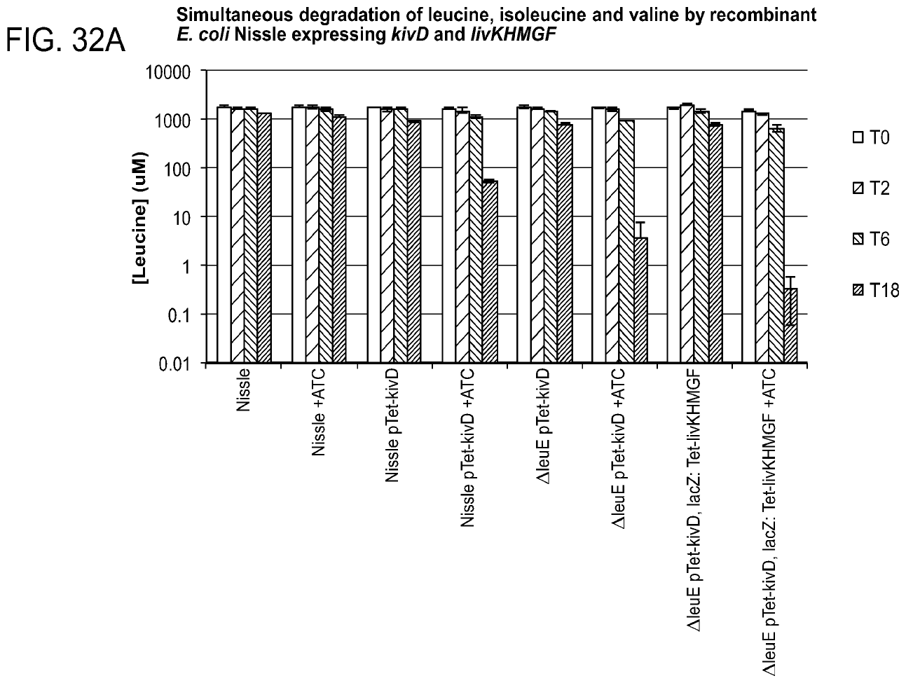

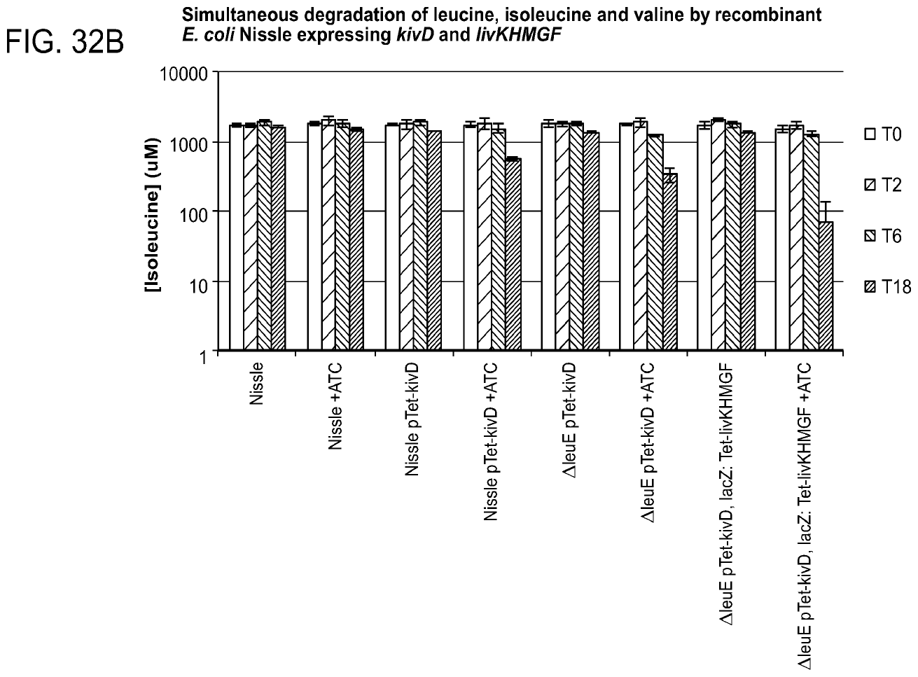

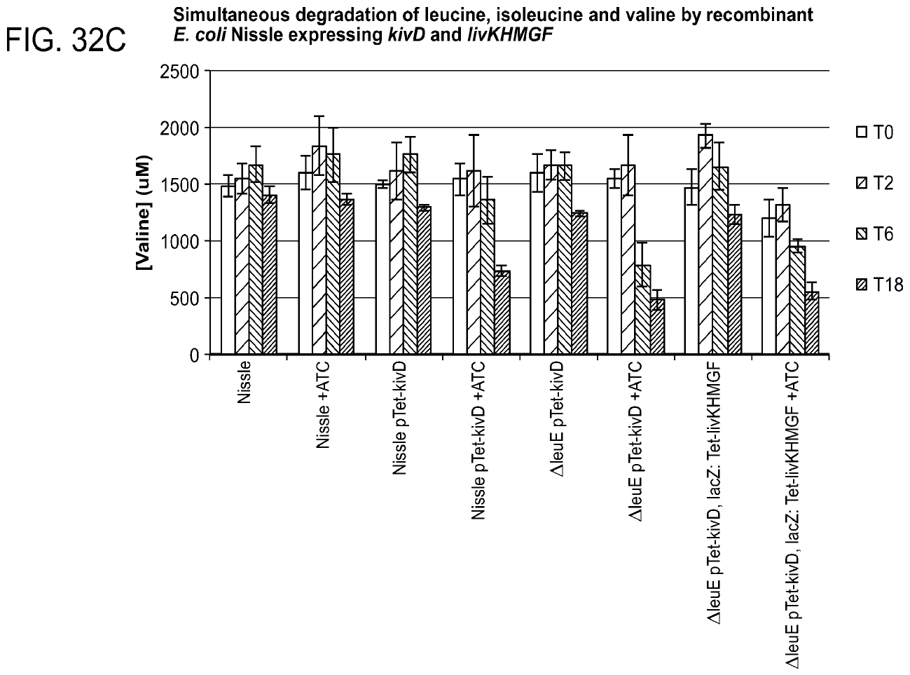

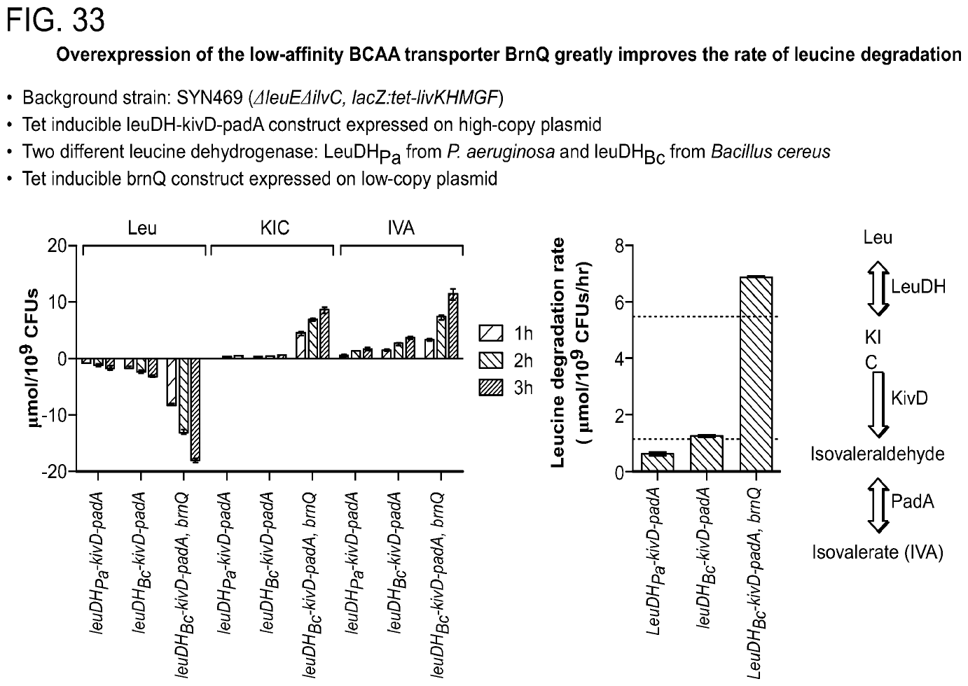

As shown in FIGS. 32A-32C, leucine, isoleucine and valine were all degraded by the expression of kivD in E. coli Nissle. At 18 h, 96.8%, 67.2% and 52.1% of leucine, isoleucine and valine respectively were degraded in Nissle expressing kivD in the presence of ATC. The efficiency of leucine and isoleucine degradation was further improved by expressing kivD in the ΔleuE background strain with a 99.8% leucine and 80.6% isoleucine degradation at 18 h. Finally, an additional increase in leucine and isoleucine degradation was achieved by expressing the leucine transporter livKHMGF in the Nissle ΔleuE pTet-kivD strain with a 99.98% leucine and 95.5% isoleucine degradation at 18 h. No significant improvement in valine degradation was observed in the ΔleuE deletion strain expressing livKHMGF.

### Example 10. Increase of BCAA Import by Overexpressing the High Affinity BCAA Transporters livKHMGF and livJHMGF In Vitro

### Study Objective

BCAA accumulate to toxic levels in MSUD patients
Different synthetic probiotic E. coli Nissle strains were engineered to efficiently import BCAA into the bacterial cell to be degraded
The objective of this study was to determine if expressing the two BCAA transporters livKHMGF and livJHMGF increase the import of valine, a BCAA naturally secreted to high levels by E. coli Nissle.

### Description of the Different Probiotic Strains.

All strains are derived from the human probiotic strain E. coli Nissle 1917. A ΔleuE deletion strain (deleted for the leucine exporter leuE) was generated by lambda red-recombination.
A copy of the high-affinity leucine ABC transporter livKHMGF under the control of a tetracycline-inducible promoter (Ptet) was inserted into the lacZ focus of the ΔleuE deletion strain by lambda-red recombination to generate the ΔleuE, lacZ: Ptet-livKHMGF strain. In this strain, the BCAA transporter livKHMGF can get induced in the presence of anhydrotetracycline (ATC).
Finally, the endogenous promoter of livJ was swapped with the constitutive promoter Ptac by lambda-red recombination to generate the ΔleuE, lacZ: Ptet-livKHMGF, Ptac-livJ strain. In this strain, livJ is constitutively induced. In the presence of ATC, both BCAA transporters livKHMGF and livJHMGF are expressed

### Experimental Procedure

The three strains tested (ΔleuE; ΔleuE, lacZ: Ptet-livKHMGF; ΔleuE, lacZ:Ptet-livKHMGF, Ptac-livJ) were grown overnight at 37° C. and 250 rpm in 4 mL of LB
Cells were diluted 100 fold in 4 mL LB and grown for 2 h at 37° C. and 250 rpm
Cells were split in two 2 mL culture tubes
One 2 mL culture tube was induced with 100 ng/mL anhydrotetracycline (ATC) to activate the Ptet promoter.
After 1 h induction, 1 mL of cells was spun down at maximum speed for 30 seconds in a microcentrifuge.
The supernatant was removed and the pellet re-suspended in 1 mL M9 medium 0.5% glucose
The cells were spun down again at maximum speed for 30 seconds and resuspended in 1 mL M9 medium 0.5% glucose.
The cells were transferred to a culture tube and incubated at 37° C. and 250 rpm for 5.5 h
150 μL of cells were collected at Oh, 2 h and 5.5 h.
The concentration of valine in the cell supernatant at the different time points was determined by LC-MS/MS.

### Results

The natural secretion of valine by E. coli Nissle is observed for the ΔleuE strain. The secretion of valine is strongly reduced for ΔleuE, lacZ: Ptet-livKHMGF in the presence of ATC. This strongly suggests that the secreted valine is efficiently imported back into the cell by livKHMGF. The secretion of valine is abolished in the ΔleuE, lacZ: Ptet-livKHMGF, Ptac-livJ strain, with or without ATC. This strongly suggests that the constitutive expression of livJ is sufficient to import back the entire amount of valine secreted by the cell via the livJHMGF transporter. E. coli Nissle was engineered to efficiently import BCAA, in this case valine, using both an inducible promoter (Ptet), and a constitutive promoter (Ptac), controlling the expression of livKHMGF and livJ respectively.

### Example 11: Generation of Bacterial Strains with Enhance Ability to Transport Biomolecules

Due to their case of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing an amino acid is disrupted a strain capable of high-affinity capture of said amino acid can be evolved via ALE. First, the strain is grown in varying concentrations of the auxotrophic amino acid, until a minimum concentration to support growth is established. The strain is then passaged at that concentration, and diluted into lowering concentrations of the amino acid at regular intervals. Over time, cells that are most competitive for the amino acid—at growth-limiting concentrations—will come to dominate the population. These strains will likely have mutations in their amino acid-transporters resulting in increased ability to import the essential and limiting amino acid.

Similarly, by using an auxotroph that cannot use an upstream metabolite to form an amino acid, a strain can be evolved that not only can more efficiently import the upstream metabolite, but also convert the metabolite into the essential downstream metabolite. These strains will also evolve mutations to increase import of the upstream metabolite, but may also contain mutations which increase expression or reaction kinetics of downstream enzymes, or that reduce competitive substrate utilization pathways.

In the previous examples, a metabolite innate to the microbe was made essential via mutational auxotrophy and selection was applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate. Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

These methods were used to generate E. coli Nissle mutants that consume kynurenine and over-produce tryptophan as described elsewhere herein.

### Example 12: Engineered Bacteria Engineered to Efficiently Import KYN

In the tumor microenvironment the amino acid tryptophan (TRP) and its degradation product kynurenine (KYN) play pivotal roles as immunomodulatory signals. Tumors often degrade TRP (which has proinflammatory properties) into KYN, which possesses anti-inflammatory characteristics, thereby promoting evasion from immune surveillance.

E. coli Nissle can be engineered to efficiently import KYN and convert it to TRP. While Nissle does not typically utilize KYN, by introducing the Kynureninase (KYNase) from Pseudomonas fluorescens (kynU) on a medium-copy plasmid under the control of the tetracycline promoter (Ptet) a new strain with this plasmid (Ptet-KYNase) is able to convert L-kynurenine into anthranilate.

E. coli naturally utilizes anthranilate in its TRP biosynthetic pathway. Briefly, the TrpE (in complex with TrpD) enzyme converts chorismate into anthranilate. TrpD, TrpC, TrpA and TrpB then catalyze a five-step reaction ending with the condensation of an indole with serine to form tryptophan. By replacing the TrpE enzyme via lambda-RED recombincering, the subsequent strain of Nissle (AtrpE::Cm) is an auxotroph unable to grow in minimal media without supplementation of TRP or anthranilate. By expressing kynureninase in ΔtrpE::Cm (KYNase-trpE), this auxotrophy can be alternatively rescued by providing KYN.

Leveraging the growth-limiting nature of KYN in KYNase-trpE, adaptive laboratory evolution was employed to evolve a strain capable of increasingly efficient utilization of KYN. First a lower limit of KYN concentration was established and mutants were evolved by passaging in lowering concentrations of KYN. While this can select for mutants capable of increasing KYN import, the bacterial cells still prefer to utilize free, exogenous TRP. In the tumor environment, dual-therapeutic functions can be provided by depletion of KYN and increasing local concentrations of TRP. Therefore, to evolve a strain which prefers KYN over TRP, a toxic analogue of TRP-5-fluoro-L-tryptophan (ToxTRP)—can be incorporated into the ALE experiment. The resulting best performing strain is then whole genome sequenced in order to deconvolute the contributing mutations. Lambda-RED can be performed in order to reintroduce TrpE, to inactivate Trp regulation (trpR, tyrR, transcriptional attenuators) to up-regulate TrpABCDE expression and increase chorismate production. The resulting strain is now insensitive to external TRP, efficiently converts KYN into TRP, and also now overproduces TRP.

### Kynureninase Protein Sequences

| Description | ID | Sequence |
|---|---|---|
| Pseudomonas | P83788 | MTTRNDCLALDAQDSLAPLRQQFALPEGVIYLDGNS |
| kynureninase | | LGARPVAALARAQAVIAEEWGNGLIRSWNSAGWRD |
| | | LSERLGNRLATLIGARDGEVVVTDTTSINLFKVLSAA |
| | | LRVQATRSPERRVIVTETSNFPTDLYIAEGLADMLQQ |
| | | GYTLRLVDSPEELPQAIDQDTAVVMLTHVNYKTGYM |
| | | HDMQALTALSHECGALAIWDLAHSAGAVPVDLHQA |
| | | GADYAIGCTYKYLNGGPGSQAFVWVSPQLCDLVPQP |
| | | LSGWFGHSRQFAMEPRYEPSNGIARYLCGTQPITSLA |
| | | MVECGLDVFAQTDMASLRRKSLALTDLFIELVEQRC |
| | | AAHELTLVTPREHAKRGSHVSFEHPEGYAVIQALIDR |
| | | GVIGDYREPRIMRFGFTPLYTTFTEVWDAVQILGEILD |
| | | RKTWAQAQFQVRHSVT* |
| Human | Q16719 | MEPSSLELPADTVQRIAAELKCHPTDERVALHLDEED |
| | | KLRHFRECFYIPKIQDLPPVDLSLVNKDENAIYFLGNS |
| | | LGLQPKMVKTYLEEELDKWAKIAAYGHEVGKRPWI |
| | | TGDESIVGLMKDIVGANEKEIALMNALTVNLHLLML |
| | | SFFKPTPKRYKILLEAKAFPSDHYAIESQLQLHGLNIE |
| | | ESMRMIKPREGEETLRIEDILEVIEKEGDSIAVILFSGV |
| | | HFYTGQHFNIPAITKAGQAKGCYVGFDLAHAVGNVE |
| | | LYLHDWGVDFACWCSYKYLNAGAGGIAGAFIHEKH |
| | | AHTIKPALVGWFGHELSTRFKMDNKLQLIPGVCGFRI |
| | | SNPPILLVCSLHASLEIFKQATMKALRKKSVLLTGYLE |
| | | YLIKHNYGKDKAATKKPVVNIITPSHVEERGCOLTITF |
| | | SVPNKDVFQELEKRGVVCDKRNPNGIRVAPVPLYNS |
| | | FHDVYKFTNLLTSILDSAETKN* |
| Shewanella | Q8E973 | MLLNVKQDFCLAGPGYLLNHSVGRPLKSTEQALKQA |
| | | FFAPWQESGREPWGQWLGVIDNFTAALASLENGQPQ |
| | | DFCPQVNLSSALTKIVMSLDRLTRDLTRNGGAVVLM |
| | | SEIDFPSMGFALKKALPASCELRFIPKSLDVTDPNVW |
| | | DAHICDDVDLVFVSHAYSNTGQQAPLAQIISLARERG |
| | | CLSLVDVAQSAGILPLDLAKLQPDFMIGSSVKWLCSG |
| | | PGAAYLWVNPAILPECQPQDVGWFSHENPFEFDIHDF |
| | | RYHPTALRFWGGTPSIAPYAIAAHSIEYFANIGSQVM |
| | | REHNLQLMEPVVQALDNELVSPQEVDKRSGTIILQFG |
| | | ERQPQILAALAAANISVDTRSLGIRVSPHIYNDEADIA |
| | | RLLGVIKANR* |

*designates the position of the stop codon

Selected codon-optimized sequences for Kynureninase

| Kynureninase | |
|---|---|
| protein sequences | Kynureninase protein sequences |
| Ptet-kynU | atctaatctagacatcattaattcctaatttttgttgacactctatcattgatagagttat |
| (Pseudomonas) | tttaccactccctatcagtgatagagaaaagtgaattatataaaagtgggaggtgccc |
| | gaatgacgacccgaaatgattgcctagcgttggatgcacaggacagtctggctccgctg |
| | cgccaacaatttgcgctgccggagggtgtgatatacctggatggcaattcgctgggcgca |
| | cgtccggtagctgcgctggctcgcgcgcaggctgtgatcgcagaagaatggggcaacg |
| | ggttgatccgttcatggaactctgcgggctggcgtgatctgtctgaacgcctgggtaatcg |
| | cctggctaccctgattggtgcgcgcgatggggaagtagttgttactgataccacctcgatt |
| | aatctgtttaaagtgctgtcagcggcgctgcgcgtgcaagctacccgtagcccggagcg |
| | ccgtgttatcgtgactgagacctcgaatttcccgaccgacctgtatattgcggaagggttg |
| | gcggatatgctgcaacaaggttacactctgcgtttggtggattcaccggaagagctgcca |
| | caggctatagatcaggacaccgcggtggtgatgctgacgcacgtaaattataaaaccggt |
| | tatatgcacgacatgcaggctctgaccgcgttgagccacgagtgtggggctctggcgatt |
| | tgggatctggcgcactctgctggcgctgtgccggtggacctgcaccaagcgggcgcgg |
| | actatgcgattggctgcacgtacaaatacctgaatggcggcccgggttcgcaagcgtttgt |
| | ttgggtttcgccgcaactgtgcgacctggtaccgcagccgctgtctggttggttcggccat |
| | agtcgccaattcgcgatggagccgcgctacgaaccttctaacggcattgctcgctatctgt |
| | gcggcactcagcctattactagcttggctatggtggagtgcggcctggatgtgtttgcgca |
| | gacggatatggcttcgctgcgccgtaaaagtctggcgctgactgatctgttcatcgagctg |
| | gttgaacaacgctgcgctgcacacgaactgaccctggttactccacgtgaacacgcgaa |
| | acgcggctctcacgtgtcttttgaacaccccgagggttacgctgttattcaagctctgattg |
| | atcgtggcgtgatcggcgattaccgtgagccacgtattatgcgtttcggtttcactcctctgt |
| | atactacttttacggaagtttgggatgcagtacaaatcctgggcgaaatcctggatcgtaag |
| | acttgggcgcaggctcagtttcaggtgcgccactctgttacttaaaaataaaacgaaag |
| | gctcagtcgaaagactgggcctttcgttttatctgttg |
| Ptet-kynU | atctaatctagacatcattaattcctaatttttgttgacactctatcattgatagagttat |
| (Human) | tttaccactccctatcagtgatagagaaaagtgaatatcaagacacgaggaggtaag |
| | attatggagccttcatctttagaactgccagcggacacggtgcagcgcatcgcggcgga |
| | actgaagtgccatccgactgatgagcgtgtggcgctgcatctggacgaagaagataaac |
| | tgcgccactttcgtgaatgtttttatattcctaaaattcaagacttgccgccggtagatttgagt |
| | ctcgttaacaaagatgaaaacgcgatctactttctgggcaactctctgggtctgcaaccaa |
| | aaatggttaaaacgtacctggaggaagaactggataaatgggcaaaaatcgcggcttatg |
| | gtcacgaagtgggcaagcgtccttggattactggcgacgagtctattgtgggtttgatgaa |
| | agatattgtgggcgcgaatgaaaaggaaattgcactgatgaatgctctgaccgttaatctg |
| | cacctgctgatgctgtctttttttaaaccgaccccgaaacgctacaaaatactgctggaagc |
| | gaaagcgtttccgtcggatcactatgctatagaaagtcaactgcagttgcatggtctgaata |
| | tcgaggaatctatgcgcatgattaaaccgcgtgagggtgaagaaacgctgcgtattgaag |
| | acattctggaagttattgaaaaagaaggtgattctatcgcagttatactgttttctggcgtgca |
| | cttttatacaggtcagcacttcaatatcccggcaatcactaaagcggggcaggcaaaagg |
| | ctgctatgttggttttgacctggcgcatgcagtggggaatgttgaactgtatctgcacgattg |
| | gggcgttgatttcgcgtgttggtgtagctacaaatatctgaacgctggcgcgggggcatt |
| | gctggcgcttttattcacgaaaaacacgcgcacaccattaaaccggctctggttggctggt |
| | tcggtcatgagctgagtactcgctttaaaatggataacaaactgcaattgattccgggtgttt |
| | gcggcttccgtatcagcaatccgccgattctgctggtttgcagcctgcacgctagtctgga |
| | aatctttaagcaggcgactatgaaagcgctgcgcaaaaaatctgtgctgctgaccggctat |
| | ctggagtatctgatcaaacacaattatggcaaagataaagctgcaactaaaaaaccggta |
| | gtgaacattatcaccccctcacacgtggaggagcgcggttgtcagctgactattactttca |
| | gtgtacctaataaagatgtgttccaggaactggaaaaacgcggcgttgtttgtgataaacg |
| | taacccgaatggtattcgcgtggctcctgtgccgctgtacaattcattccacgatgtttataa |
| | attcaccaacctgctgacttctattctcgacagtgctgagactaaaaattaaaaataaaac |
| | gaaaggctcagtcgaaagactgggcctttcgttttatctgttg |
| ptet-kynU | atctaatctagacatcattaattcctaatttttgttgacactctatcattgatagagttat |
| (Shewanella) | tttaccactccctatcagtgatagagaaaagtgaatggttcaccaccacaaggaggg |
| | attatgctgctgaatgtaaaacaggacttttgcctggcaggcccgggctacctgctgaatc |
| | actcggttggccgtccgctgaaatcaactgagcaagcgctgaaacaagcattttttgctcc |
| | gtggcaagagagcggtcgtgaaccgtggggccagtggctgggtgttattgataatttcac |
| | tgctgcgctggcatctctgtttaatggtcaaccgcaggatttttgtccgcaggttaacctgag |
| | cagcgcgctgactaaaattgtgatgtcactggatcgtctgactcgcgatctgacccgcaat |
| | ggcggtgctgttgtgctgatgtctgaaatcgatttcccatctatgggcttcgcgttgaaaaa |
| | agcgctgccagcgagctgcgaactgcgttttatcccgaaaagtctggacgtgactgatcc |
| | gaacgtatgggatgcacacatctgtgatgatgtagacctggtttttgtgtctcacgcctatag |
| | taatacgggccaacaggctccgctggcgcaaatcatctctctggcgcgtgaacgtggct |
| | gcctgtcactggtggatgtagcgcaatcagcggggattttgccgctggatctggcgaaac |
| | tgcaaccggacttcatgatcggcagttcggttaaatggctgtgctcgggccctggtgcgg |
| | catatctgtgggttaatccggcgattctgccggaatgtcagccgcaggatgtgggctggtt |
| | ttcacatgagaatccctttgaattcgacatccacgatttccgctaccacccgactgcactgc |
| | gcttttggggggtacgccgtcgatcgcgccttatgcgatcgcggcgcactcgatcgaat |
| | attttgccaatatcggctcgcaagtgatgcgtgaacacaacctgcaactgatggaaccggt |
| | ggttcaggcgctggacaatgaactggtgagcccgcaggaagtggataaacgctcaggc |
| | actattattctgcaattcggtgaacgtcaaccgcaaattctggcggctctggctgcggcga |
| | acatttcggtggacactcgttctttggggattcgtgttagtccgcacatttataatgatgagg |
| | cggacattgcgcgcctgctgggtgtgatcaaagcaaatcgctaaaaataaaacgaaagg |
| | ctcagtcgaaagactgggcctttcgttttatctgttg |

The ptet-promoter is in bold, designed Ribosome binding site is underlined, codon-optimized protein coding sequence is in plain text, and the terminator is in italics.

Generation of E. coli Mutants with increased ability to consume L-Kynurenine

### Example 13. Results

Adaptive Laboratory Evolution was used to produce mutant bacterial strains that consume Kynurenine and produce tryptophan. First, a ΔtrpE strain was constructed that expresses kynureninase and is capable of converting L-kynurenine to anthranilate to rescue the auxotrophic tryptophan background (KYNase). E. coli Nissle can be engineered to efficiently import KYN and convert it to TRP. While Nissle does not typically utilize KYN, by introducing the Kynureninase (KYNase) from Pseudomonas fluorescens (kynU) on a medium-copy plasmid under the control of the tetracycline promoter (Ptet) a new strain with this plasmid (Ptet-KYNase) is able to convert L-kynurenine into anthranilate.

E. coli naturally utilizes anthranilate in its TRP biosynthetic pathway. Briefly, the TrpE (in complex with TrpD) enzyme converts chorismate into anthranilate. TrpD, TrpC, TrpA and TrpB then catalyze a five-step reaction ending with the condensation of an indole with serine to form tryptophan. By replacing the TrpE enzyme via lambda-RED recombineering, the subsequent strain of Nissle (ΔtrpE::Cm) is an auxotroph unable to grow in minimal media without supplementation of TRP or anthranilate. By expressing kynureninase in ΔtrpE::Cm (KYNase-trpE), this auxotrophy can be alternatively rescued by providing KYN.

First a lower limit of KYN concentration was established and mutants were evolved by passaging in lowering concentrations of KYN. While this can select for mutants capable of increasing KYN import, the bacterial cells still prefer to utilize free, exogenous TRP. In the tumor environment, dual-therapeutic functions can be provided by depletion of KYN and increasing local concentrations of TRP. Therefore, to evolve a strain which prefers KYN over TRP, a toxic analogue of TRP-5-fluoro-L-tryptophan (ToxTRP)—can be incorporated into the ALE experiment. The resulting best performing strain is then whole genome sequenced in order to deconvolute the contributing mutations. Lambda-RED can be performed in order to reintroduce TrpE, to inactivate Trp regulation (trpR, tyrR, transcriptional attenuators) to up-regulate TrpABCDE expression and increase chorismate production. The resulting strain is now insensitive to external TRP, efficiently converts KYN into TRP, and also now overproduces TRP.

To establish the minimum concentration of L-kynurenine and maximum concentration of 5-fluoro-L-tryptophan (Tox Trp) capable of sustaining growth of the KYNase strain, using a checkerboard assay, the following protocol was used. Using a 96-well plate with M9 minimal media with glucose, KYNU is supplemented decreasing across columns in 2-fold dilutions from 2000 μg/mL down to ˜1 μg/mL. In the rows, Tox Trp concentration decreases by 2-fold from 200 μg/mL down to ˜1.5 ug/mL. In one plate, Anhydrous Tetracycline (aTc) was added to a final concentration of 100 ng/uL to induce production of the KYNase. From an overnight culture cells were diluted to an OD600=0.5 in 12 mL of TB (plus appropriate antibiotics and inducers, where applicable) and grown for 4 hours. 100 μL of cells were spun down and resuspended to an OD600=1.0. These were diluted 2000-fold and 25 uL was added to each well to bring the final volumes in each well to 100 uL. Cells were grown for roughly 20 hours with static incubation at 37 C then growth was assessed by OD600, making sure readings fell within linear range (0.05-1.0).

Once identified, the highest concentrations of Tox Trp and lowest concentration of kynurenine capable of supporting growth becomes the starting point for ALE. The ALE parental strain was chosen by culturing the KYNase strain on M9 minimal media supplemented with glucose and L-kynurenine (referred to as M9+KYNU from here on). A single colony was selected, resuspended in 20 μL of sterile phosphate-buffered saline solution. This colony was then used to inoculate three cultures of M9+KYNU, grown into late-logarithmic phase and optical density determined at 600 nm. These cultures were then diluted to 103 in 4 rows of a 96-well deep-well plate with 1 mL of M9+KYNU. Each one of the four rows has a different ToxTrp (increasing 2-fold), while each column has decreasing concentrations of KYNU (by 2-fold). Each morning and evening this plate is diluted back to 103 using the well in which the culture has grown to just below saturation so that the culture is always in logarithmic growth. This process is repeated until a change in growth rate is no longer detected. Once no growth rate increases are detected (usually around 10¹¹ Cumulative Cell Divisions) the culture is plated onto M9+KYNU. Phillips, R. S. Structure and mechanism of kynureninase. Archives of Biochemistry and Biophysics 544, 69-74 (2014). Individual colonies are selected and screened in M9+KYNU+ToxTrp media to confirm increased growth rate phenotype. Once mutants with significantly increased growth rate on M9+KYNU are isolated, genomic DNA can be isolated and sent for whole genome sequencing to reveal the mutations responsible for phenotype.

All culturing is done shaking at 350 RPM at 37° C.

| | Rich | Min | Min + | Min + |
|---|---|---|---|---|
| STRAIN | Media | Media | Anthranilate | KYNU + aTc |
| SYN094 | + | + | + | + |
| trpE | + | − | + | − |
| trpE | + | − | + | + |
| pseudoKYNase | | | | |
| trpE hKYNase | + | − | + | − |

In a preliminary assay, wildtype Nissle (SYN094), Nissle with a deletion of trpE, and trpE mutants expressing either the human kynureninase (hKYNase) or the Pseudomonas fluorescens kynureninase (pseudoKYNase) from a Ptet promoter on a medium-copy plasmid were grown in either rich media, minimal media (min media), minimal media with 5 mM anthranilate (Min+anthranilate) or minimal media with 10 mM kynurenine and 100 ng/uL aTc (Min+KYNU+aTc). These were grown in 1 mL of media in a deep well plate with shaking at 37° C. A positive for growth (+) in the above table indicates a change in optical density of >5-fold from inoculation.

The results show that in a mutant trpE (which is typically used in the tryptophan biosynthetic pathway to convert chorismate into anthranilate) background, Nissle is unable to grow in minimal media without supplementation with anthranilate (or tryptophan). When minimal media was supplemented with KYNU, the trpE mutant was also unable to grow. However, when the pseudoKYNase was expressed in the trpE tryptophan-auxotroph the cells were able to grow in Min+KYNU. This indicates that Nissle is able to import L-kynurenine from the media and convert it into anthranilate using the pseudoKYNase. The hKYNase homolog was unable to support growth on M9+KYNU, most likely due to differences in substrate specificity as it has been documented that the human kynureninase prefers 3-hydroxykynurenine as a substrate. Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

Moving forward with the knowledge that Nissle is able to grow on KYNU supplemented minimal media in a trpE auxotroph by importing and converting kynurenine, the next step was to establish the minimal concentrations of kynurenine capable of supporting growth. Additionally, in our selection experiment if 5-fluoro-L-tryptophan (ToxTrp) was employed the concentrations of both KYNU and ToxTrp capable of still sustaining growth. A growth assay was performed in 96-well plates using SYN094, trpE and trpE pseudoKYNase with and without induction of pseudoKYNase expression using 100 ng/uL aTc. These strains were inoculated at very dilute concentrations into M9 minimal media with varying concentrations of KYNU across columns (2-fold dilutions starting at 2000 μg/mL) and varying concentrations of Tox Trp across rows (2-fold dilutions starting at 200 μg/mL). On a separate plate, the strains were grown in M9+KYNU (at the same concentrations) in the absence of ToxTrp.

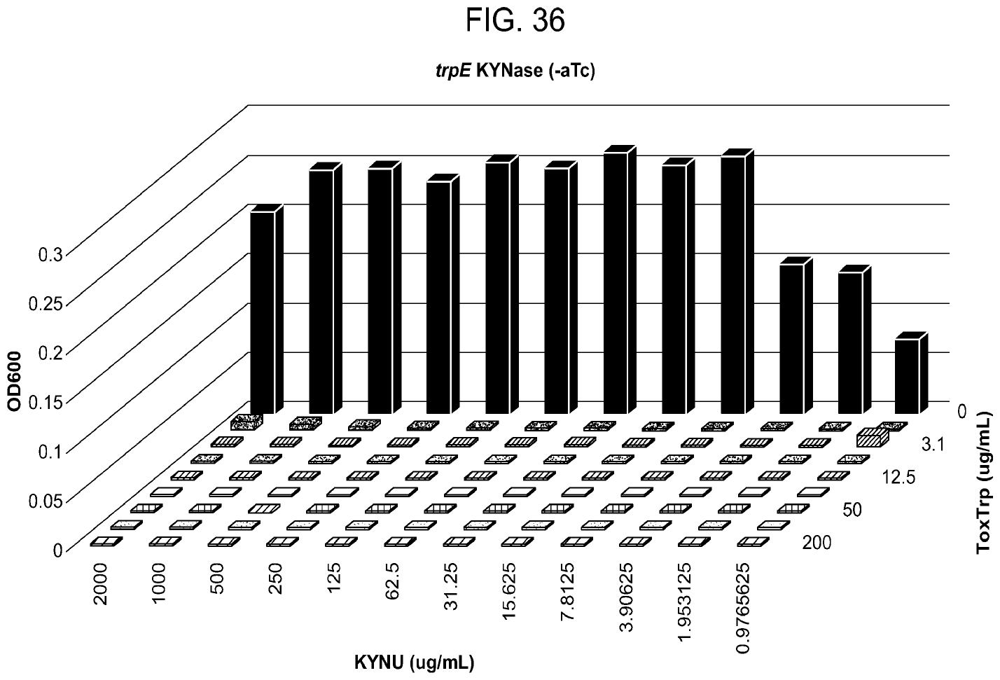

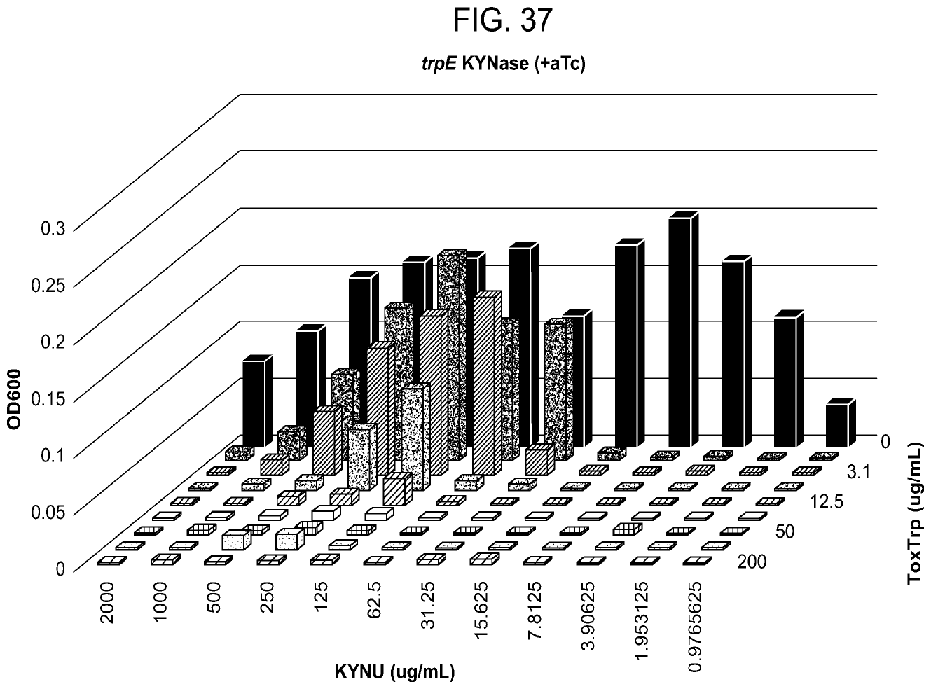

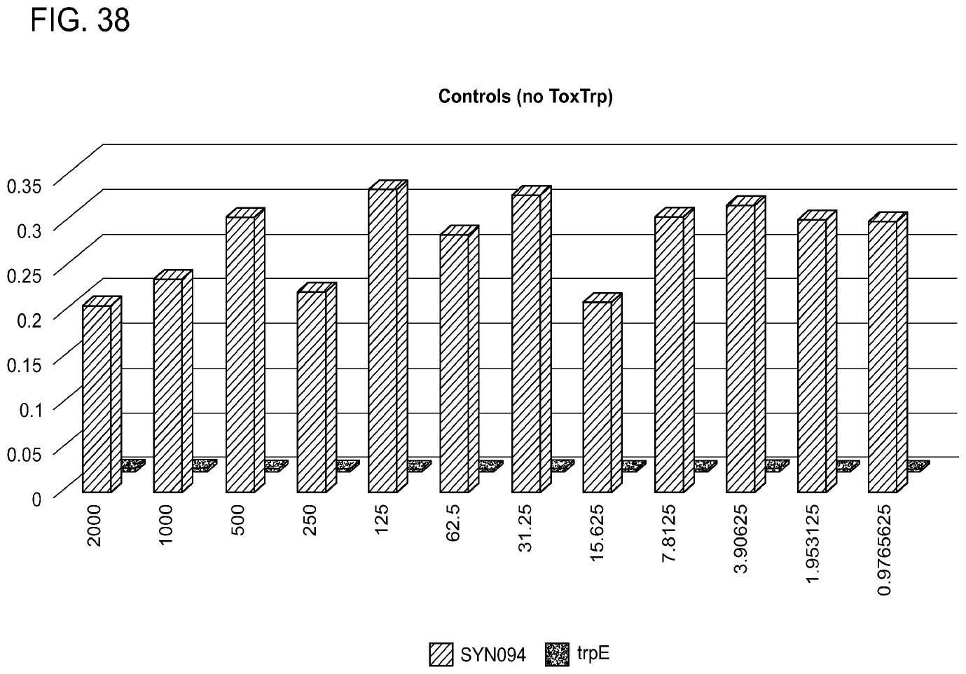

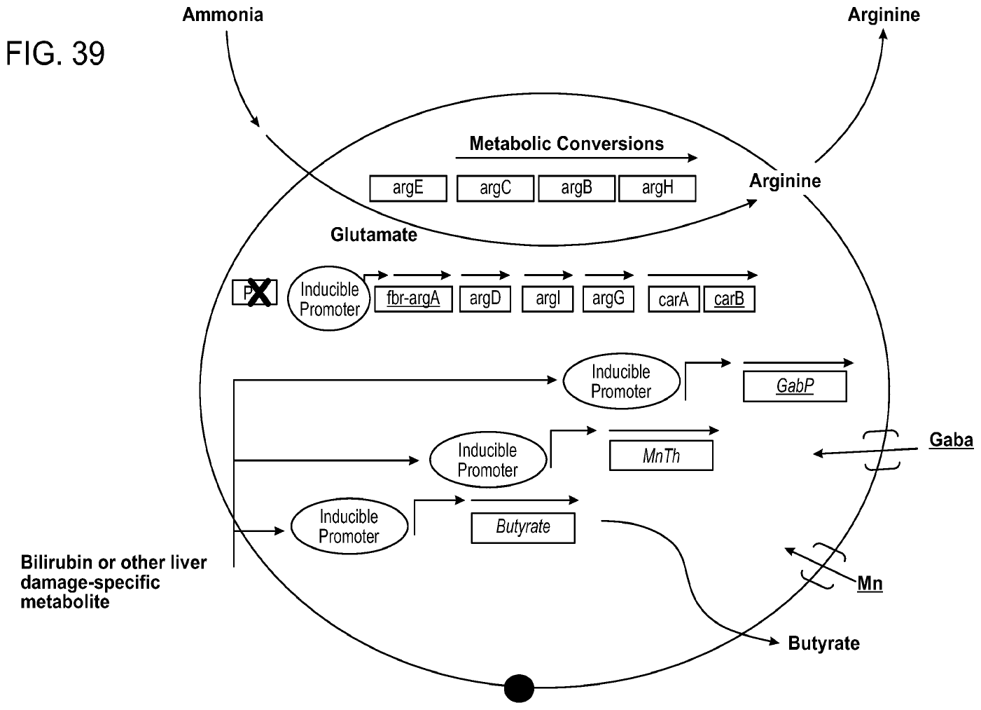

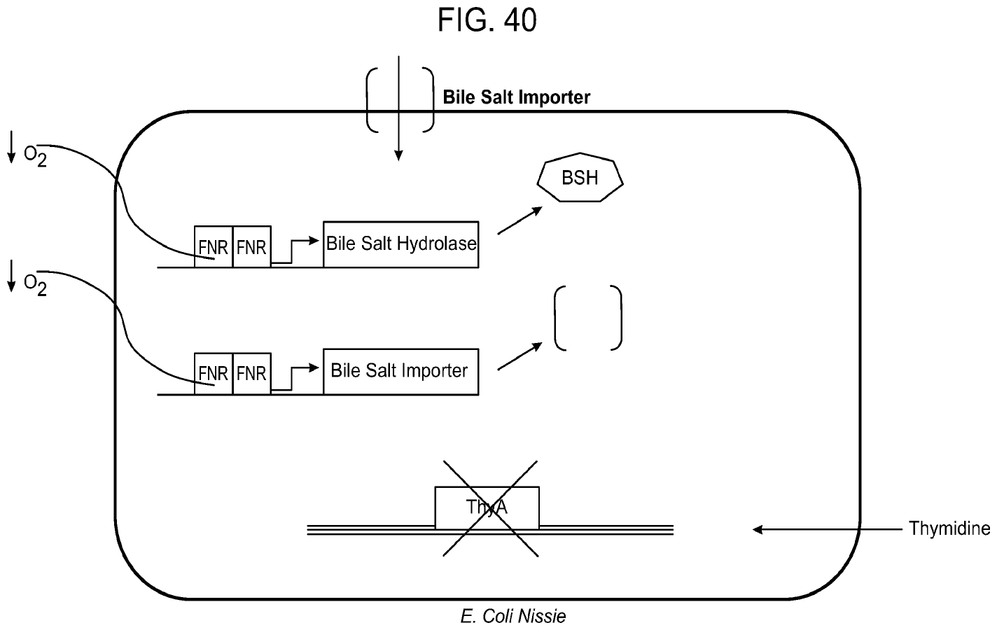

The results of the initial checkerboard assay are displayed in FIGS. 36-38 as a function of optical density at 600 nm (normalized to a media blank). In FIGS. 36 and 37, the X-axis shows decreasing KYNU concentration from left-to-right, while the Z-axis shows decreasing Tox Trp concentration from front-to-back with the very back row representing media with no ToxTrp. In FIG. 38. the control strains SYN094 and trpE are shown in M9+KYNU without any ToxTrp, as there was no growth detected from either strain at any concentration of ToxTrp. The results of the assay show that expression of the pseudoKYNase provides protection against toxicity of Tox Trp. More importantly, growth is permitted between 250-62.5 ug/mL of KYNU and 6.3-1.55 ug/mL of ToxTrp.

Together these experiments establish that expression of the Pseudomonas fluorescens kynureninase is sufficient to rescue a trpE auxotrophy in the presence of kynurenine. In addition, the pseudoKYNase is also capable of providing increased resistance to the toxic tryptophan, 5-fluoro-L-tryptophan. Using the information attained here it is possible to proceed to an adaptive laboratory evolution experiment to select for mutants with highly efficient and selective conversion of kynurenine to tryptophan.

1.-23. (canceled)

24. A method of producing a pharmaceutically acceptable composition comprising a genetically-engineered non-pathogenic bacterium for use in metabolizing phenylalanine, the bacterium comprising
at least one heterologous gene encoding a phenylalanine transporter for importing phenylalanine into the bacterium operably linked to a directly or indirectly inducible promoter that is not associated with the phenylalanine transporter in nature, and
at least one heterologous gene encoding a polypeptide involved in metabolizing phenylalanine operably linked to a directly or indirectly inducible promoter that is not associated with the at least one heterologous gene encoding the polypeptide in nature,
the method comprising the steps of:
a) growing the bacterium in a growth medium culture under conditions that induce the promoter that controls expression of the transporter for importing phenylalanine;
b) isolating the resulting bacteria from the growth medium; and
c) suspending the isolated bacteria in a pharmaceutically acceptable carrier.

25. The method of claim 24, wherein the promoter operably linked to the at least one heterologous gene encoding the phenylalanine transporter and the promoter operably linked to the at least one heterologous gene encoding the polypeptide are separate copies of the same promoter.

26. The method of claim 24, wherein the promoter operably linked to the at least one heterologous gene encoding the phenylalanine transporter and the promoter operably linked to the at least one gene encoding the polypeptide are the same copy of the same promoter.

27. The method of claim 24, wherein the promoter operably linked to the at least one heterologous gene encoding the phenylalanine transporter and the promoter operably linked to the at least one heterologous gene encoding the polypeptide are directly or indirectly induced by exogenous environmental conditions.

28. The method of claim 27, wherein the exogenous environmental conditions are exogenous environmental conditions found in the gut of a mammal.

29. The method of claim 27, wherein the exogenous environmental conditions are exogenous environmental conditions found in the microenvironment of a tumor.

30. The method of claim 27, wherein the exogenous environmental conditions are low-oxygen or anaerobic conditions.

31. The method of claim 24, wherein the promoter operably linked to the at least one heterologous gene encoding the phenylalanine transporter and the promoter operably linked to the at least one heterologous gene encoding the polypeptide are selected from the group consisting of an FNR-responsive promoter, an ANR-responsive promoter, and a DNR-responsive promoter.

32. The method of claim 24, wherein the at least one heterologous gene encoding the phenylalanine transporter is located on a chromosome in the bacterium.

33. The method of claim 24, wherein the at least one heterologous gene encoding the phenylalanine transporter is located on a plasmid in the bacterium.

34. The method of claim 24, wherein the at least one heterologous gene encoding the polypeptide is located on a plasmid in the bacterium.

35. The method of claim 24, wherein the at least one heterologous gene encoding the polypeptide is located on a chromosome in the bacterium.

36. The method of claim 24, wherein the bacterium is a probiotic bacterium.

37. The method of claim 36, wherein the bacterium is selected from the group consisting of Bacteroides, Bifidobacterium, Clostridium, Escherichia, Lactobacillus, and Lactococcus.

38. The method of claim 37, wherein the bacterium is Escherichia coli strain Nissle.

39. The method of claim 24, wherein the bacterium is an auxotroph in a gene that is complemented when the bacterium is present in a human gut.

40. The method of claim 39, wherein the bacterium is an auxotroph in diaminopimelic acid or an enzyme in the thymidine biosynthetic pathway.
